(19) United States
(12) Patent Application Publication
(10) Pub. No.: US 2024/0166635 A1
(43) Pub. Date: May 23, 2024

(54) AMINOHETEROARYL KINASE INHIBITORS

(71) Applicant: Anrui Biomedical Technology (Guangzhou) Co.,Ltd., Guangzhou, Guangdong (CN)

(72) Inventors: Dai Cheng, Guangzhou (CN); Qiang Ding, Guangzhou (CN); Zhixiang He, Guangzhou (CN); Xiaobo Zhou, Guangzhou (CN); Yang Zhou, Guangzhou (CN); Xiaohang Yin, Guangzhou (CN); Zeqiang Xie, Guangzhou (CN)

(21) Appl. No.: 18/254,573

(22) PCT Filed: Nov. 26, 2021
(86) PCT No.: PCT/CN2021/133429

§ 371 Date: May 25, 2023

(30) Foreign Application Priority Data
Nov. 27, 2020 (WO) PCT/CN2020/132454
Mar. 17, 2021 (WO) PCT/CN2021/081236

(51) Int. Cl. C07D 405/12; C07D 239/42; C07D 239/47; C07D 401/04; C07D 401/12; C07D 401/14; C07D 403/04; C07D 405/14

(52) U.S. Cl. CPC C07D 405/12; C07D 239/42; C07D 239/47; C07D 401/04; C07D 401/12; C07D 401/14; C07D 403/04; C07D 405/14

(57) ABSTRACT

Provided herein are novel compounds (e.g., Formula I or II), pharmaceutical compositions, and methods of using related to cyclin dependent kinases (CDKs). The compounds herein are typically CDK2 inhibitors, which can be used for treating a variety of diseases or disorders, such as cancer.

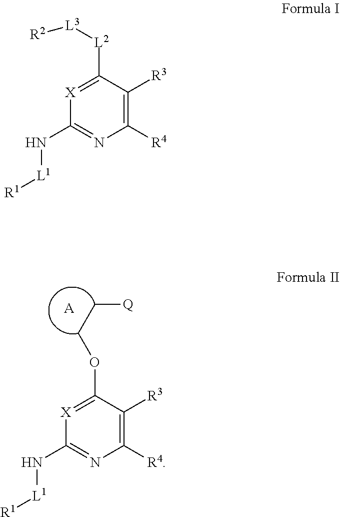

### CROSS REFERENCE TO RELATED APPLICATIONS

This application claims priority of International Application Nos. PCT/CN2021/081236, filed Mar. 17, 2021 and PCT/CN2020/132454, filed Nov. 27, 2020, the content of each of which is incorporated herein by reference in its entirety for all purposes.

In various embodiments, the present disclosure generally relates to novel heteroaryl compounds, compositions comprising the same, methods of preparing and methods of using the same, e.g., for inhibiting cyclin-dependent kinases and/or for treating or preventing various diseases or disorders described herein.

### BACKGROUND

Cyclin-dependent kinase (CDKs) are a family of serine/threonine protein kinases that regulate the cell cycle progression. Among CDKs, CDK2 is an essential driver for cells to transition from late G1 into S and G2 phases. During late G1, CDK2 is activated upon binding to cyclin E. The cyclin E/CDK2 complex hyper-phosphorylates RB to release E2F from Rb and initiate transcription of genes necessary for G1/S transition. Subsequently, CDK2 forms complex with Cyclin A to regulate S phase progression by activating proteins important for DNA replication and centrosome duplication, such as DNA replication licensing protein (CDC6) and centrosome protein CP110 (Tadesse et al. Targeting CDK2 in cancer: challenges and opportunities for therapy, Drug Discovery Today. 2019; 25(2): 406-413).

Cyclin E1 is frequently amplified and/or overexpressed in human cancer. In high grade serous ovarian cancer, cyclin E1 amplification is detected in approximately 20% of patients and is associated with chemo resistance/refractory (TCGA, Integrated genomic analyses of ovarian carcinoma, Nature. 2011; 474: 609-615; Nakayama et al; Gene amplification CCNE1 is related to poor survival and potential therapeutic target in ovarian cancer, Cancer (2010) 116: 2621-34). Cyclin E1 amplified ovarian cancer cell lines are sensitive to reagents that either inhibit CDK2 activity or decrease cellular CDK2 protein level, suggesting CDK2 dependence in these cyclin E1 amplified cells (Au-Yeung et al. Selective targeting of cyclin E1 amplified high grade serous ovarian cancer by clin-dependent kinase 2 and AKT inhibition, Clin. Cancer Res. 2017; 23(7):1862-1874). Poor outcomes and drug resistance were also associated with high Cyclin E1 expression in endometrial, gastric, breast and other cancers (Noske et al., Detection of CCNE1/URI (19q12) amplification by in situ hybridization is common in high grade and type II endometrial cancer, Oncotarget (2017) 8: 14794-14805; Ooi et al., Gene amplification of CCNE1, CCND1 and CDK6 in gastric cancers detected by multiplex ligation-dependent probe amplification and fluorescence in situ hybridization, Hum Pathol. (2017) 61:58-67; Keyomarsi et al., Cyclin E and survival in patients with breast cancer. N Engl J Med. (2002) 347: 1566-75). Estrogen receptor (ER) positive breast cancer cell lines with acquired resistance to CDK4/6 inhibitor Palbociclib has elevated cyclin E1 expression and can be re-sensitized upon knock down of CDK2 (Herrera-Abreu et al., Early adaptation and acquired resistance to CDK4/6 inhibition in estrogen receptor-positive breast cancer, Cancer Res. (2016) 76: 2301-2313). High cyclin E1 level was also reported to associate with poor response to Palbociclib plus fulvestrant combo therapy in ER+BC (CCNE1 high vs CCNE1 low: median PFS for Palbociclib+fulvestrant arm, 7.6 v 14.1 month; placebo+fulvestrant arm, 4.0 v 4.8 month) further underline the importance of CDK2 activity in mediating resistance to CDK4/6 inhibitors (Turner et al., Cyclin E1 expression and Palbociclib efficacy in previously treated hormone receptor positive metastatic breast cancer Clin Oncol. (2019) 37(14): 1169-1178).

Cyclin E2 (CCNE2) overexpression was reported as associated with endocrine resistance in breast cancer cells and CDK2 inhibition has been reported to restore sensitivity to tamoxifen or CDK4 inhibitors in tamoxifen-resistant and CCNE2 overexpressing cells. (Caldon et al., Cyclin E2 overexpression is associated with endocrine resistance but not insensitivity to CDK2 inhibition in human breast cancer cells. Mol Cancer Ther. (2012) 11:1488-99; Herrera-Abreu et al., Early Adaptation and Acquired Resistance to CDK4/6 Inhibition in Estrogen Receptor-Positive Breast Cancer, Cancer Res. (2016) 76: 2301-2313). Additionally, Cyclin E amplification has also been reported as contributing to trastuzumab resistance in HER2+ breast cancer. (Scaltriti et al. Cyclin E amplification/overexpression is a mechanism of trastuzumab resistance in HER2+ breast cancer patients, Proc Natl Acad Sci. (2011) 108: 3761-6). Further, Cyclin E overexpression was reported to play a role in basal-like and triple negative breast cancer (TNBC), as well as inflammatory breast cancer. (Elsawaf & Sinn, Triple Negative Breast Cancer: Clinical and Histological Correlations, Breast Care (2011) 6:273-278; Alexander et al., Cyclin E overexpression as a biomarker for combination treatment strategies in inflammatory breast cancer, Oncotarget (2017) 8: 14897-14911.)

### BRIEF SUMMARY

The importance of CDK2 in proliferative pathways and the frequently altered CDK2/cyclin E1 activity in tumor highlights CDK2 as a target for cancer treatment. CDK2 knock out mice are viable with minimum defects, suggesting CDK2 is not essential for normal cell proliferation (Berthet et al., CDK2 knock out mice are viable. Curr Biol. (2003) 13(20):1775-85). In addition, selective CDK2 inhibitors may minimize clinical toxicity while being active in treating patients with high tumor cyclin E1 and/or E2 expression. However, in some embodiments, inhibiting CDK2 as well as other CDKs can also be clinically beneficial.

In various embodiments, the present disclosure relates to novel heteroaryl compounds which can inhibit CDK2, e.g., selectively over other CDKs and/or other kinases. The compounds and compositions herein are useful for treating various diseases or disorders, such as cancer, e.g., those characterized with amplification or overexpression of Cyclin E1 (CCNE1) and/or cyclin E2 (CCNE2).

Some embodiments of the present disclosure are directed to a compound of Formula I or II, or a pharmaceutically acceptable salt thereof,

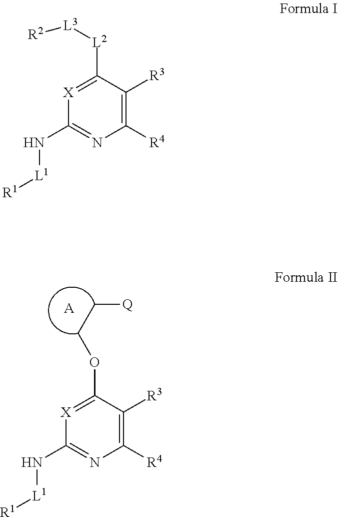

wherein the variables are defined herein. In some embodiments, the compound of Formula I can have a sub-formula of I-1, I-2, I-3, I-4, I-5, I-2-1, I-2-1-S1, I-2-1-S2, I-2-1-S3, I-2-1-S4, I-5-1, I-5-2, I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B, I-A-8B, I-A-9B, I-A-10B, or I-B, as defined herein. In some embodiments, the compound of Formula II can have a sub-formula of II-A, II-1, II-2, II-1-S1, II-1-S2, II-1-S3, II-1-S4, II-2-S1, II-2-S2, II-2-S3, or II-2-S4, as defined herein. In some embodiments, the present disclosure also provides specific compounds selected from any of Examples 1-155, or any of the specific compounds disclosed in Table 1A or 1B herein, or a pharmaceutically acceptable salt thereof.

In some embodiments, the present disclosure provides a pharmaceutical composition comprising one or more compounds of the present disclosure and optionally a pharmaceutically acceptable excipient. The pharmaceutical composition can be typically formulated for oral administration.

In some embodiments, the present disclosure also provides a method of inhibiting CDK activity such as CDK2 activity in a subject or biological sample. In some embodiments, the method comprises contacting the subject or biological sample with an effective amount of one or more compounds of the present disclosure, e.g., a compound of Formula I (e.g., I-1, I-2, I-3, I-4, I-5, I-2-1, I-2-1-S1, I-2-1-S2, I-2-1-S3, I-2-1-S4, I-5-1, I-5-2, I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B, I-A-8B, I-A-9B, I-A-10B, or I-B), Formula II (e.g., II-A, II-1, II-2, II-1-S1, II-1-S2, II-1-S3, II-1-S4, II-2-S1, II-2-S2, II-2-S3, or 11-2-S4), any of Examples 1-155, or any of the specific compounds disclosed in Table 1A or 1B herein, or a pharmaceutically acceptable salt thereof, or a pharmaceutical composition comprising the same.

In some embodiments, the present disclosure provides a method of treating or preventing a CDK-mediated disease or disorder in a subject in need thereof. In some embodiments, the method comprises administering to the subject an effective amount of one or more compounds of the present disclosure or the pharmaceutical composition herein. In some embodiments, the method comprises administering to the subject an effective amount of a compound of Formula I (e.g., I-1, I-2, I-3, I-4, I-5, I-2-1, I-2-1-S1, I-2-1-S2, I-2-1-S3, I-2-1-S4, I-5-1, I-5-2, I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B, I-A-8B, I-A-9B, I-A-10B, or I-B), Formula II (e.g., II-A, II-1, II-2, II-1-S1, II-1-S2, II-1-S3, II-1-S4, II-2-S1, II-2-S2, II-2-S3, or II-2-S4), any of Examples 1-155, or any of the specific compounds disclosed in Table 1A or 1B herein, or a pharmaceutically acceptable salt thereof, or a pharmaceutical composition comprising the same.

In some embodiments, the present disclosure also provides a method of treating or preventing cancer in a subject in need thereof, which comprises administering to the subject an effective amount of a compound of the present disclosure (e.g., a compound of Formula I (e.g., I-1, I-2, I-3, I-4, I-5, I-2-1, I-2-1-S1, I-2-1-S2, I-2-1-S3, I-2-1-S4, I-5-1, I-5-2, I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B, I-A-8B, I-A-9B, I-A-10B, or I-B), Formula II (e.g., II-A, II-1, II-2, II-1-S1, II-1-S2, II-1-S3, II-1-S4, II-2-S1, II-2-S2, II-2-S3, or II-2-S4), any of Examples 1-155, or any of the specific compounds disclosed in Table 1A or 1B herein, or a pharmaceutically acceptable salt thereof) or an effective amount of a pharmaceutical composition described herein. In some embodiments, the cancer is characterized by amplification or overexpression of CCNE1 and/or CCNE2. In some embodiments, the cancer is selected from breast cancer, ovarian cancer, bladder cancer, uterine cancer, prostate cancer, lung cancer (including NSCLC, SCLC, squamous cell carcinoma or adenocarcinoma), esophageal cancer, head and neck cancer, colorectal cancer, kidney cancer (including RCC), liver cancer (including HCC), pancreatic cancer, stomach (i.e., gastric) cancer, thyroid cancer, and combinations thereof. In some embodiments, the cancer is breast cancer selected from ER-positive/HR-positive, HER2-negative breast cancer; ER-positive/HR-positive, HER2-positive breast cancer; triple negative breast cancer (TNBC); and inflammatory breast cancer. In some embodiments, the cancer is breast cancer. In some embodiments, the cancer is breast cancer selected from endocrine resistant breast cancer, trastuzumab resistant breast cancer, or breast cancer demonstrating primary or acquired resistance to CDK4/CDK6 inhibition. In some embodiments, the cancer is advanced or metastatic breast cancer. In some embodiments, the cancer is ovarian cancer.

The administering in the methods herein is not limited to any particular route of administration. For example, in some embodiments, the administering can be orally, nasally, transdermally, pulmonary, inhalationally, buccally, sublingually, intraperintoneally, subcutaneously, intramuscularly, intravenously, rectally, intrapleurally, intrathecally and parenterally. In some embodiments, the administering is orally. In some embodiments, the administering is a parenteral injection, such as an intraveneous injection.

Compounds of the present disclosure can be used as a monotherapy or in a combination therapy. In some embodiments according to the methods described herein, one or more compounds of the present disclosure can be administered as the only active ingredient(s). In some embodiments, the method herein further comprises administering to the subject an additional therapeutic agent, such as additional anticancer agents described herein.

It is to be understood that both the foregoing summary and the following detailed description are exemplary and explanatory only, and are not restrictive of the invention herein.

### DETAILED DESCRIPTION

In various embodiments, the present disclosure provides compounds and compositions that are useful for inhibiting CDKs such as CDK2 and/or treating or preventing various diseases or disorders described herein, e.g., cancer.

Compounds

The compounds of the present disclosure are generally aminopyridine or aminopyrimidine derivatives having a Formula I or II described herein. The compounds herein can typically inhibit CDK2. In some embodiments, the compounds herein can selectively inhibit CDK2 over other CDKs. For example, as shown in the Examples section herein, certain exemplified compounds were shown to be more potent in inhibiting CDK2 over CDK1, with a selectivity of more than 10-fold, and up to about 30-fold and beyond.

Formula I

In some embodiments, the present disclosure provides a compound of Formula I, or a pharmaceutically acceptable salt thereof:

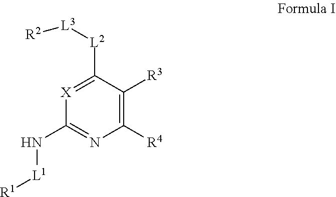

wherein:
L¹ is an optionally substituted arylene (e.g., phenylene), optionally substituted heteroarylene (e.g., 5- or 6-membered heteroarylene), optionally substituted heterocyclylene (e.g., 4-8-membered heterocyclylene), or optionally substituted carbocyclylene (e.g., C₃₋₈ carbocyclylene);
R¹ is SO₂R¹⁰, SO₂NR¹¹R¹², S(O)(NH)R¹⁰, or C(O)NR¹¹R¹²; or R¹ is hydrogen or NR¹¹R¹²;
X is N or CR¹³;
L² is a bond, —N(R¹⁴)—, or —O—;
L³ is a bond, an optionally substituted C₁₋₄ alkylene or an optionally substituted C₁₋₄ heteroalkylene;
R² is hydrogen, an optionally substituted C₃₋₈ alkyl, optionally substituted C₃₋₈ carbocyclyl, optionally substituted 4-10 membered heterocyclyl, optionally substituted phenyl, or optionally substituted 5-10 membered heteroaryl;
R³ is hydrogen, halogen (e.g., F), CN, C(O)NR¹¹R¹², optionally substituted C₁₋₆ alkyl, optionally substituted C₂₋₄ alkenyl, optionally substituted C₂₋₄ alkynyl, optionally substituted C₁₋₄ heteroalkyl, OR^{A}, COR^{B}, COOR^{A} NR¹¹R¹², optionally substituted C₃-s carbocyclyl, optionally substituted 4-10 membered heterocyclyl, or optionally substituted 5-10 membered heteroaryl;
R⁴ is hydrogen, halogen (e.g., F), optionally substituted C₁₋₆ alkyl, or NR¹¹R¹²; or L² and R³, together with the intervening atoms, form an optionally substituted 4-8 membered ring structure; or R³ and R⁴, together with the intervening atoms, form an optionally substituted 4-8 membered ring structure;
wherein:
R¹⁰ is an optionally substituted C₁₋₆ alkyl (e.g., C₁₋₄ alkyl optionally substituted with a carbocyclec, heterocycle or heteroaryl), optionally substituted C₃-s carbocyclyl, optionally substituted phenyl, optionally substituted heteroaryl (e.g., 5- or 6-membered heteroaryl), or optionally substituted 4-10 membered heterocyclyl;
each of R¹¹ and R¹², at each occurrence, is independently hydrogen, an optionally substituted C₁₋₆ alkyl, optionally substituted C₃-s carbocyclyl, optionally substituted phenyl, optionally substituted heteroaryl (e.g., 5- or 6-membered heteroaryl), optionally substituted 4-10 membered heterocyclyl; or a nitrogen protecting group; or R¹¹ and R¹² can be joined to form an optionally substituted 4-10 membered heterocyclyl or 5- or 6-membered heteroaryl;
R^{A} is hydrogen, an optionally substituted C₁₋₆ alkyl, optionally substituted C₃-s carbocyclyl, optionally substituted phenyl, optionally substituted heteroaryl (e.g., 5- or 6-membered heteroaryl), optionally substituted 4-10 membered heterocyclyl; or an oxygen protecting group;
R^{B} is hydrogen, an optionally substituted C₁₋₆ alkyl, optionally substituted C₃₋₈ carbocyclyl, optionally substituted phenyl, optionally substituted 4-10 membered heterocyclyl, or optionally substituted heteroaryl (e.g., 5- or 6-membered heteroaryl);
R¹³ is hydrogen, F, CN, —OH, an optionally substituted C₁₋₄ alkyl, optionally substituted C₁₋₄ heteroalkyl, optionally substituted C₃-s carbocyclyl, or optionally substituted 4-10 membered heterocyclyl; and
R¹⁴ is hydrogen, an optionally substituted C₁₋₆ alkyl, optionally substituted C₃₋₈ carbocyclyl, optionally substituted phenyl, optionally substituted heteroaryl (e.g., 5- or 6-membered heteroaryl), optionally substituted 4-10 membered heterocyclyl; or a nitrogen protecting group.

In some embodiments, the compound of Formula I (including any of the applicable sub-formulae as described herein) can comprise one or more asymmetric centers and/or axial chirality, and thus can exist in various stereoisomeric forms, e.g., enantiomers and/or diastereomers. In some embodiments, the compound of Formula I can exist in the form of an individual enantiomer and/or diastereomer, as applicable, or a mixture of stereoisomers, including racemic mixtures and mixtures enriched in one or more stereoisomers. In some embodiments, when applicable, the compound of Formula I (including any of the applicable sub-formulae as described herein) can exist as an isolated individual enantiomer substantially free (e.g., with less than 20%, less than 10%, less than 5%, less than 1%, by weight, by HPLC or SFC area, or both, or with a non-detectable amount) of the other enantiomer. In some embodiments, when applicable, the compound of Formula I (including any of the applicable sub-formulae as described herein) can also exist as a mixture of stereoisomers in any ratio, such as a racemic mixture.

In some embodiments, the compound of Formula I (including any of the applicable sub-formulae as described herein) can exist as an isotopically labeled compound, particularly, a deuterated analog, wherein one or more of the hydrogen atoms of the compound of Formula I is/are substituted with a deuterium atom with an abundance above its natural abundance, e.g., a CD₃ analog when the compound has a CH₃ group.

It should be apparent to those skilled in the art that in certain cases, the compound of Formula I may exist as a mixture of tautomers. The present disclosure is not limited to any specific tautomer. Rather, the present disclosure encompasses any and all of such tautomers whether or not explicitly drawn or referred to.

Typically, X in Formula I is N, and the compound of Formula I can be characterized as having Formula I-A:

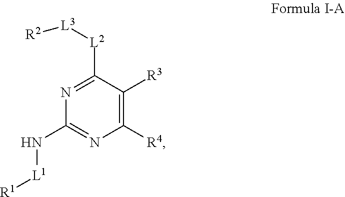

wherein L¹, L², L³, R¹, R², R³, and R⁴ include any of those described herein in any combination.

In some embodiments, X in Formula I can be CR¹³, wherein R¹³ is defined herein. For example, in some embodiments, R¹³ can be hydrogen, and the compound of Formula I can be characterized as having Formula I-B:

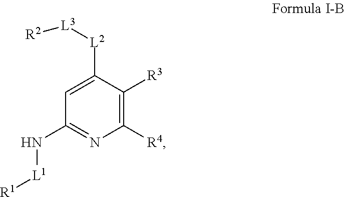

wherein L¹, L², L³, R¹, R², R³, and R⁴ include any of those described herein in any combination.

Various groups are suitable as L¹ in Formula I. For example, in some embodiments, L¹ in Formula I can be an optionally substituted phenylene. In some embodiments, L¹ in Formula I can be an optionally substituted 5- or 6-membered heteroarylene, e.g., those having 1-3 ring heteroatoms independently selected from N, O, and S. In some embodiments, L¹ in Formula I can be an optionally substituted 4-8-membered heterocyclylene, e.g., a monocyclic or bicyclic (e.g., fused, bridged, or spiro bicyclic) 4-8 membered heterocyclylene having 1-2 ring heteroatoms independently selected from N, O, and S. In some embodiments, L¹ in Formula I can be an optionally substituted C₃₋₈ carbocyclylene, e.g., a monocyclic or bicyclic (e.g., fused, bridged, or spiro bicyclic) carbocyclylene.

In some specific embodiments, L¹ in Formula I (e.g., any of the subformulae described herein as applicable, such as Formula I-1, I-2, I-3, I-4, I-5, I-2-1, I-2-1-S1, I-2-1-S2, I-2-1-S3, I-2-1-S4, I-5-1, I-5-2, I-A, or I-B) is selected from:

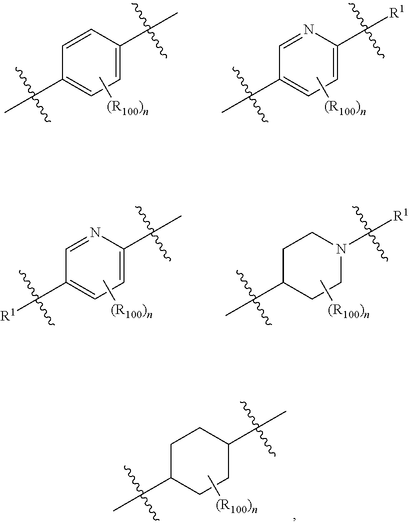

wherein:
n is 0, 1, 2, 3, or 4, as valency permits; and
R¹⁰⁰ at each occurrence is independently selected from halogen (e.g., F or Cl), CN, OH, optionally substituted C₁₋₄ alkyl, optionally substituted C₁₋₄ alkoxy, and optionally substituted C₁₋₄ heteroalkyl. Typically, n is 0, 1, or 2.

In some embodiments, L¹ in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, or I-B) is unsubstituted phenylene, pyridylene, piperidinylene, or cyclohexylene. For example, in some embodiments, L¹ is:

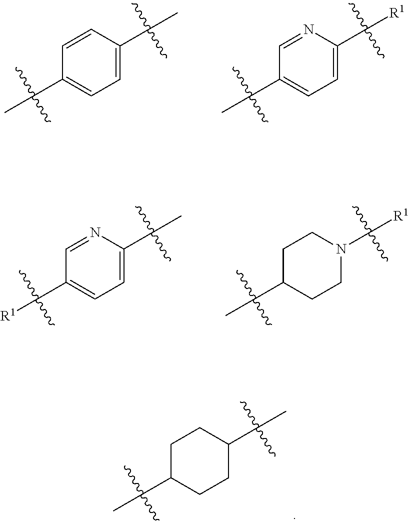

In some specific embodiments, L¹ in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, or I-B) is selected from:

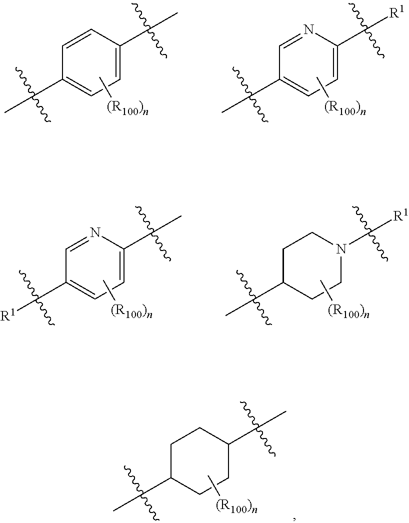

wherein
n is 1 or 2; and
R¹⁰⁰ at each occurrence is independently selected from F, Cl, CN, OH, C₁₋₄ alkyl optionally substituted with F, C₁₋₄ alkoxy optionally substituted with F, and C₁₋₄ heteroalkyl optionally substituted with F.

In some embodiments, L¹ in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, or I-B) is a phenylene, pyridylene, piperidinylene, or cyclohexylene, each of which can be optionally further substituted, such as monosubstituted or disubstituted. For example, in some embodiments, L¹ in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, or I-B) is selected from:

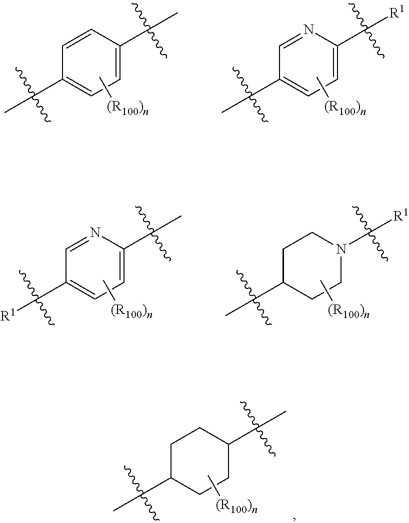

wherein:
R¹⁰⁰ is F, Cl, CN, OH, methyl, fluorine-substituted methyl such as CF₃, methoxy, or fluorine-substituted methoxy. In any of the embodiments herein, unless specified or otherwise contrary from context, L¹ in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, or I-B) can be selected from:

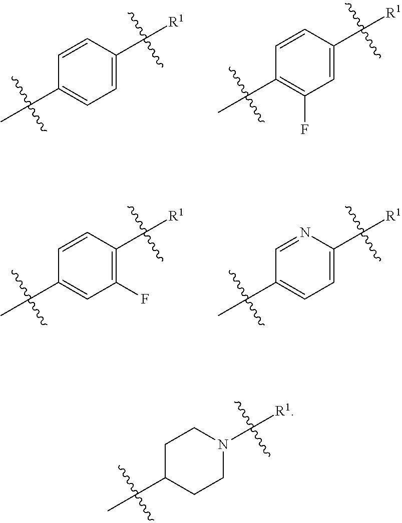

In any of the embodiments herein, unless specified or otherwise contrary from context, L¹ in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, or I-B) can be

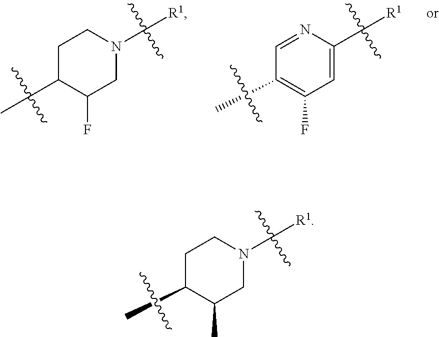

R¹ group in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, or I-B) is typically a sulfone, sulfonamide, sulfonimine, or amide. For example, in some embodiments, R¹ in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, or I- B) can be SO₂R¹⁰, wherein R¹⁰ is defined herein. In some embodiments, R¹ in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, or I-B) can be SO₂NR¹¹R¹², wherein R¹¹ and R¹² are defined herein. In some embodiments, R¹ in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, or I-B) can be S(O)(NH)R¹⁰, wherein R¹⁰ is defined herein. In some embodiments, R¹ in Formula I (e.g., Formula I-A or I-B) can be C(O)NR¹¹R¹², wherein R¹¹ and R¹² are defined herein.

In some more specific embodiments, R¹ in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, or I-B) can be SO₂R¹⁰° wherein R¹⁰ is an optionally substituted C₁₋₄ alkyl, optionally substituted C₃₋₆ cycloalkyl, or optionally substituted 4-8 membered heterocyclyl having one or two ring heteroatoms independently selected from N, O, and S. In some more specific embodiments, R¹ in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, or I-B) can be SO₂R¹⁰, wherein R¹⁰ is an optionally substituted 5 or 6 membered heteroaryl having 1-3 ring heteroatoms independently selected from N, O, and S.

In some embodiments, R¹ in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, or I-B) can be SO₂R¹⁰, wherein R¹⁰ is C₁₋₄ alkyl, (C₁₋₄ alkylene)ⱼ-C₃₋₆ cycloalkyl, or (C₁₋₄ alkylene)ⱼ-4-8 membered monocyclic heterocyclyl having one or two ring heteroatoms independently selected from N, O, and S, or R¹⁰ is (C₁₋₄ alkylene)ⱼ-(5 or 6 membered heteroaryl having 1-3 ring heteroatoms independently selected from N, O, and S),
wherein j is 0 or 1, and the C₁₋₄ alkylene is straight or branched alkyelene chain optionally substituted with F; and
wherein each of the C₁₋₄ alkyl, C₃₋₆ cycloalkyl, 5 or 6 membered heteroaryl, and 4-8 membered monocyclic heterocyclyl is optionally substituted with one or more (e.g., 1, 2, or 3) substituents independently selected from oxo, F, G¹, OH, O-G¹, NH₂, NH(G¹), and N(G¹)(G¹), wherein G¹ at each occurrence is independently a C₁₋₄ alkyl optionally substituted with 1-3 substituents independently selected from F, CN, OH, and C₁₋₄ heteroalkyl, or a C₃₋₆ cycloalkyl optionally substituted with 1-3 substituents independently selected from F, CN, OH, and C₁₋₄ heteroalkyl. In some embodiments, j is 0. In some embodiments, j is 1. In some embodiments, R¹⁰ is C₁₋₄ alkyl optionally substituted with 1-3 F, such as CH₂F, CF₃, etc. In some embodiments, R¹⁰ is —(C₁₋₄ alkylene)-C₃₋₆ cycloalkyl, for example, CH₂-cyclopropyl, which can be optionally substituted. In some embodiments, R¹⁰ is —(C₁₋₄ alkylene)-(4-8 membered monocyclic heterocyclyl), such as —CH₂-tetrahydrofuranyl, which can be optionally substituted. In some embodiments, R¹⁰ can be a 5 or 6 membered heteroaryl having 1-3 ring heteroatoms independently selected from N, O, and S, such as pyrrazole, imidazole, triazole, etc., which can be optionally substituted, for example, with a C₁₋₄ alkyl (e.g., methyl). In any of the embodiments herein, unless specified or otherwise contrary from context, R¹ in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, or I-B) can be SO₂Me. In some embodiments, R¹ in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, or I-B) can be selected from:

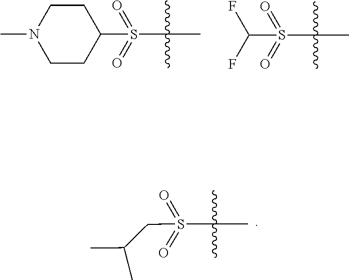

In some embodiments, R¹ in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, or I-B) can be selected from:

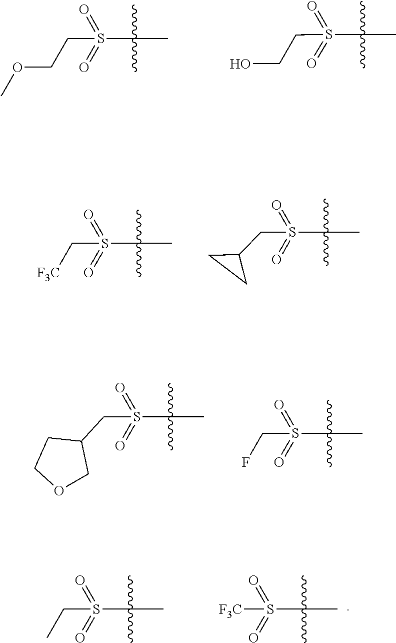

In some embodiments, R¹ in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, or I-B) can be selected from:

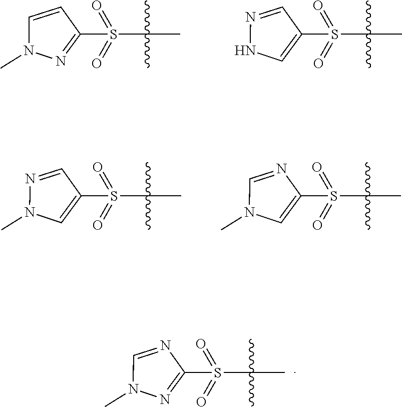

In some embodiments, R¹ in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, or I-B) can be S(O)(NH)R¹⁰, i.e.,

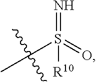

wherein R¹⁰ is an optionally substituted C₁₋₄ alkyl, optionally substituted C₃₋₆ cycloalkyl, or optionally substituted 4-8 membered heterocyclyl having one or two ring heteroatoms independently selected from N, O, and S, or an optionally substituted 5 or 6 membered heteroaryl having 1-3 ring heteroatoms independently selected from N, O, and S.

In some more specific embodiments, R¹ in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, or I-B) can be S(O)(NH)R¹⁰, i.e.,

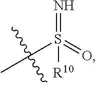

wherein R¹⁰ is C₁₋₄ alkyl, (C₁₋₄ alkylene)ⱼ-C₃₋₆ cycloalkyl, (C₁₋₄ alkylene)ⱼ-4-8 membered monocyclic heterocyclyl having one or two ring heteroatoms independently selected from N, O, and S, or R¹⁰ is (C₁₋₄ alkylene)ⱼ-(5 or 6 membered heteroaryl having 1-3 ring heteroatoms independently selected from N, O, and S),
wherein j is 0 or 1, and the C₁₋₄ alkylene is straight or branched alkyelene chain optionally substituted with F; and
wherein each of the C₁₋₄ alkyl, C₃₋₆ cycloalkyl, 5 or 6 membered heteroaryl, and 4-8 membered monocyclic heterocyclyl is optionally substituted with one or more (e.g., 1, 2, or 3) substituents independently selected from oxo, F, G¹, OH, O-G¹, NH₂, NH(G¹), and N(G¹)(G¹), wherein G¹ at each occurrence is independently a C₁₋₄ alkyl optionally substituted with 1-3 substituents independently selected from F, CN, OH, and C₁₋₄ heteroalkyl, or a C₃₋₆ cycloalkyl optionally substituted with 1-3 substituents independently selected from F, CN, OH, and C₁₋₄ heteroalkyl. In some embodiments, j is 0. In some embodiments, j is 1. In any of the embodiments herein, unless specified or otherwise contrary from context, R¹ in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, or I-B) can be S(O)(NH)Me.

In some embodiments, R¹ in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, or I-B) can be SO₂NR¹¹R¹², wherein R¹¹ and R¹² are independently hydrogen, an optionally substituted C₁₋₄ alkyl, optionally substituted C₃₋₆ cycloalkyl, or optionally substituted 4-8 membered heterocyclyl having one or two ring heteroatoms independently selected from N, O, and S. In some embodiments, one of R¹¹ and R¹² is hydrogen and the other of R¹¹ and R¹² is described herein. For example, in some embodiments, R¹ in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, or I-B) can be SO₂NR¹¹R¹², wherein one of R¹¹ and R¹² is hydrogen and the other of R¹¹ and R¹² is hydrogen, an optionally substituted C₁₋₄ alkyl, optionally substituted C₃₋₆ cycloalkyl, or optionally substituted 4-8 membered heterocyclyl having one or two ring heteroatoms independently selected from N, O, and S.

In some more specific embodiments, R¹ in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, or I-B) can be SO₂NR¹¹R¹², wherein R¹¹ and R¹² are independently hydrogen, C₁₋₄ alkyl, (C₁₋₄ alkylene)ⱼ-C₃₋₆ cycloalkyl, (C₁₋₄ alkylene)ⱼ-4-8 membered monocyclic heterocyclyl having one or two ring heteroatoms independently selected from N, O, and S, wherein j is 0 or 1, and the C₁₋₄ alkylene is straight or branched alkyelene chain optionally substituted with F; and
wherein each of the C₁₋₄ alkyl, C₃₋₆ cycloalkyl, and 4-8 membered monocyclic heterocyclyl is optionally substituted with one or more (e.g., 1, 2, or 3) substituents independently selected from oxo, deuterium, F, G¹, OH, O-G¹, NH₂, NH(G¹), and N(G¹)(G¹), wherein G¹ at each occurrence is independently a C₁₋₄ alkyl optionally substituted with 1-3 substituents independently selected from deuterium, F, CN, OH, and C₁₋₄ heteroalkyl, or a C₃₋₆ cycloalkyl optionally substituted with 1-3 substituents independently selected from deuterium, F, CN, OH, and C₁₋₄ heteroalkyl. In some embodiments, j is 0. In some embodiments, j is 1. In some embodiments, one of R¹¹ and R¹² is hydrogen and the other of R¹¹ and R¹² is described herein. In some embodiments, one of R¹¹ and R¹² is methyl or CD₃, and the other of R¹¹ and R¹² is described herein. In some embodiments, both of R¹¹ and R¹² are hydrogen. In some embodiments, one of R¹¹ and R¹² is hydrogen and the other of R¹¹ and R¹² is C₁₋₄ alkyl optionally substituted with 1-3 F and/or deuterium, such as CH₃, isopropyl, tert-butyl, CD₃, etc. In some embodiments, one of R¹¹ and R¹² is hydrogen and the other of R¹¹ and R¹² is C₃₋₆ cycloalkyl, for example, cyclopropyl or cyclobutyl, which can be optionally substituted, e.g., with one or two F. In some embodiments, one of R¹¹ and R¹² is hydrogen and the other of R¹¹ and R¹² is a 4-8 membered monocyclic heterocyclyl having 1-3 ring heteroatoms independently selected from N, O, and S, such as oxetane, tetrahydrofuran, tetrahydropyran, piperidine, etc., which can be optionally substituted, for example, with a C₁₋₄ alkyl (e.g., methyl). In some embodiments, one of R¹¹ and R¹² is hydrogen and the other of R¹¹ and R¹² is a —(C₁₋₄ alkylene)-(4-8 membered monocyclic heterocyclyl having 1-3 ring heteroatoms independently selected from N, O, and S), such as —CH₂-(oxetane), etc., which can be optionally substituted, for example, with a C₁₋₄ alkyl (e.g., methyl).

In some embodiments, R¹ in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, or I-B) can be SO₂NR¹¹R¹², wherein R¹¹ and R¹² are joined to form an optionally substituted 4-8 membered heterocyclyl having, in addition to the nitrogen atom both R¹¹ and R¹² are attached to, 0 or 1 ring heteroatom selected from N, O, and S. For example, in some embodiments, R¹ in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, or I-B) can be SO₂NR¹¹R¹², wherein R¹¹ and R¹² are joined to form a 4-8 membered monocyclic heterocyclyl having, in addition to the nitrogen atom both R¹¹ and R¹² are attached to, 0 or 1 ring heteroatom selected from N, O, and S, such as morpholinyl or piperazinyl, which is optionally substituted with one or more (e.g., 1, 2, or 3) substituents independently selected from oxo, deuterium, F, G¹, OH, O-G¹, NH₂, NH(G¹), and N(G¹)(G¹), wherein G¹ at each occurrence is independently a C₁₋₄ alkyl optionally substituted with 1-3 substituents independently selected from deuterium, F, CN, OH, and C₁₋₄ heteroalkyl, or a C₃₋₆ cycloalkyl optionally substituted with 1-3 substituents independently selected from deuterium, F, CN, OH, and C₁₋₄ heteroalkyl.

In some preferred embodiments, R¹ in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, or I-B) can be SO₂NH₂. In some preferred embodiments, R¹ in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, or I-B) can be selected from:

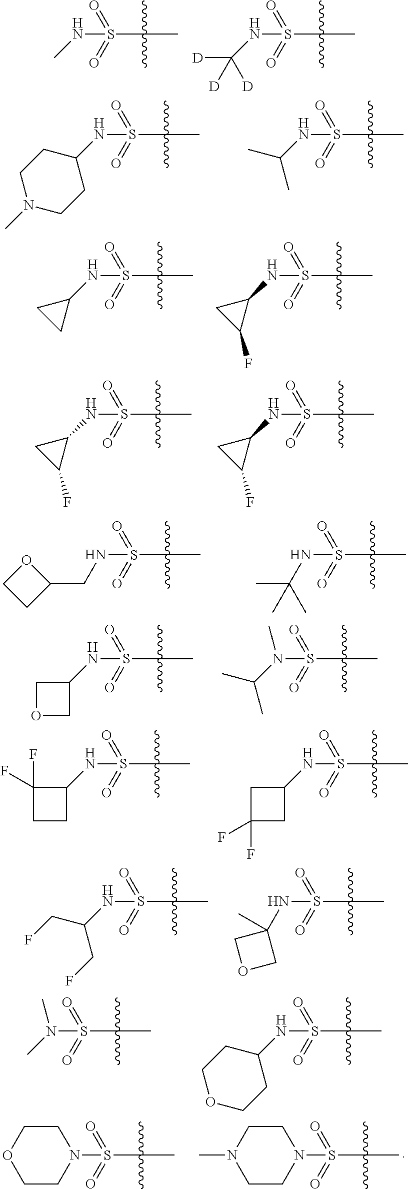

In some preferred embodiments, R¹ in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, or I-B) can be selected from:

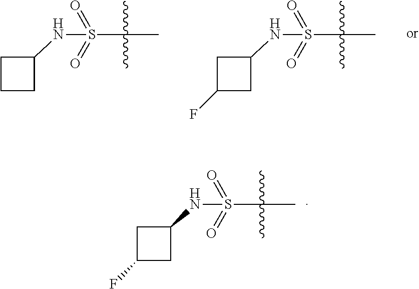 [SMILES: *S(=O)(=O)NC1CCC1 ; *S(=O)(=O)NC1CC(F)C1 ; *S(=O)(=O)N[C@H]1C[C@H](F)C1]

In some embodiments, R¹ in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, or I-B) can be C(O)NR¹¹R¹², wherein R¹¹ and R¹² are independently hydrogen, an optionally substituted C₁₋₄ alkyl, optionally substituted C₃₋₆ cycloalkyl, or optionally substituted 4-8 membered heterocyclyl having one or two ring heteroatoms independently selected from N, O, and S. In some embodiments, one of R¹¹ and R¹² is hydrogen and the other of R¹¹ and R¹² is described herein. For example, in some embodiments, R¹ in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, or I-B) can be C(O)NR¹¹R¹², wherein one of R¹¹ and R¹² is hydrogen and the other of R¹¹ and R¹² is hydrogen, an optionally substituted C₁₋₄ alkyl, optionally substituted C₃₋₆ cycloalkyl, or optionally substituted 4-8 membered heterocyclyl having one or two ring heteroatoms independently selected from N, O, and S.

For example, in some embodiments, R¹ in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, or I-B) can be C(O)NR¹¹R¹², wherein R¹¹ and R¹² are independently hydrogen, C₁₋₄ alkyl, (C₁₋₄ alkylene)ⱼ-C₃₋₆ cycloalkyl, (C₁₋₄ alkylene)ⱼ-4-8 membered monocyclic heterocyclyl having one or two ring heteroatoms independently selected from N, O, and S,
wherein j is 0 or 1, and the C₁₋₄ alkylene is straight or branched alkyelene chain optionally substituted with F; and
wherein each of the C₁₋₄ alkyl, C₃₋₆ cycloalkyl, and 4-8 membered monocyclic heterocyclyl is optionally substituted with one or more (e.g., 1, 2, or 3) substituents independently selected from oxo, deuterium, F, G¹, OH, O-G¹, NH₂, NH(G¹), and N(G¹)(G¹), wherein G¹ at each occurrence is independently a C₁₋₄ alkyl optionally substituted with 1-3 substituents independently selected from deuterium, F, CN, OH, and C₁₋₄ heteroalkyl, or a C₃₋₆ cycloalkyl optionally substituted with 1-3 substituents independently selected from deuterium, F, CN, OH, and C₁₋₄ heteroalkyl. In some embodiments, j is 0. In some embodiments, j is 1. In some embodiments, one of R¹¹ and R¹² is hydrogen and the other of R¹¹ and R¹² is described herein. For example, in some embodiments, R¹ in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, or I-B) can be C(O)NHMe.

In some embodiments, R¹ in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, or I-B) can be C(O)NR¹¹R¹², wherein R¹¹ and R¹² are joined to form an optionally substituted 4-8 membered heterocyclyl having, in addition to the nitrogen atom both R¹¹ and R¹² are attached to, 0 or 1 ring heteroatom selected from N, O, and S. For example, in some embodiments, R¹ in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, or I-B) can be C(O)NR¹¹R¹², wherein R¹¹ and R¹² are joined to form a 4-8 membered monocyclic heterocyclyl having, in addition to the nitrogen atom both R¹¹ and R¹² are attached to, 0 or 1 ring heteroatom selected from N, O, and S, which is optionally substituted with one or more (e.g., 1, 2, or 3) substituents independently selected from oxo, deuterium, F, G¹, OH, O-G¹, NH₂, NH(G¹), and N(G¹)(G¹), wherein G¹ at each occurrence is independently a C₁₋₄ alkyl optionally substituted with 1-3 substituents independently selected from deuterium, F, CN, OH, and C₁₋₄ heteroalkyl, or a C₃₋₆ cycloalkyl optionally substituted with 1-3 substituents independently selected from deuterium, F, CN, OH, and C₁₋₄ heteroalkyl. For example, in some embodiments, R¹ in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, or I-B) can be

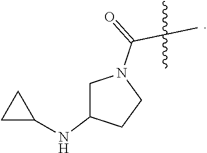 [SMILES: *C(=O)N1CCC(NC2CC2)C1]

Compounds of Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, or I-B) can have various combinations of L¹ and R¹, which are not particularly limited for the present disclosure. In any of the embodiments herein, unless specified or otherwise contrary from context, L¹-R¹ in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, or I-B) can be selected from:

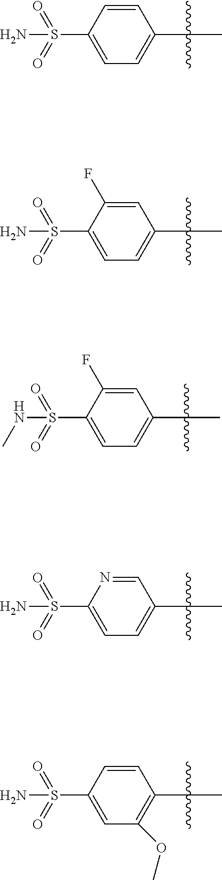 [SMILES: *c1ccc(S(N)(=O)=O)cc1 ; *c1ccc(S(N)(=O)=O)c(F)c1 ; *c1ccc(S(=O)(=O)NC)c(F)c1 ; *c1ccc(S(N)(=O)=O)nc1 ; *c1ccc(S(N)(=O)=O)cc1OC]

or L¹-R¹ is

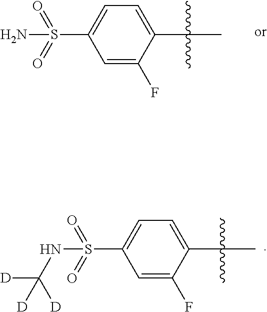 [SMILES: *c1ccc(S(N)(=O)=O)cc1F ; *c1ccc(S(=O)(=O)NC([2H])([2H])[2H])cc1F]

In any of the embodiments herein, unless specified or otherwise contrary from context, L¹-R¹ in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, or I-B) can be selected from:

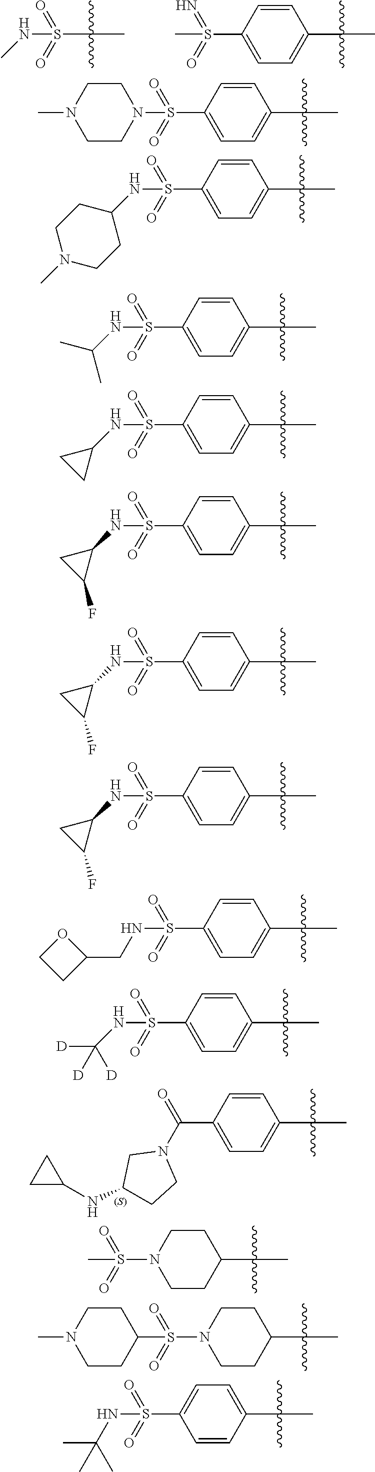

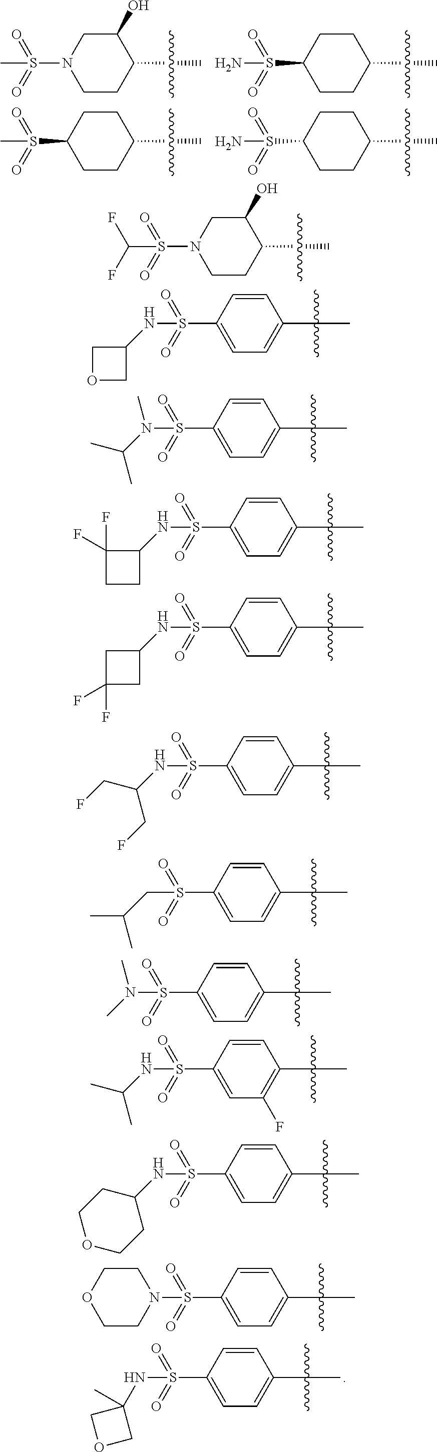

In any of the embodiments herein, unless specified or otherwise contrary from context, L¹-R¹ in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, or I-B) can be selected from:

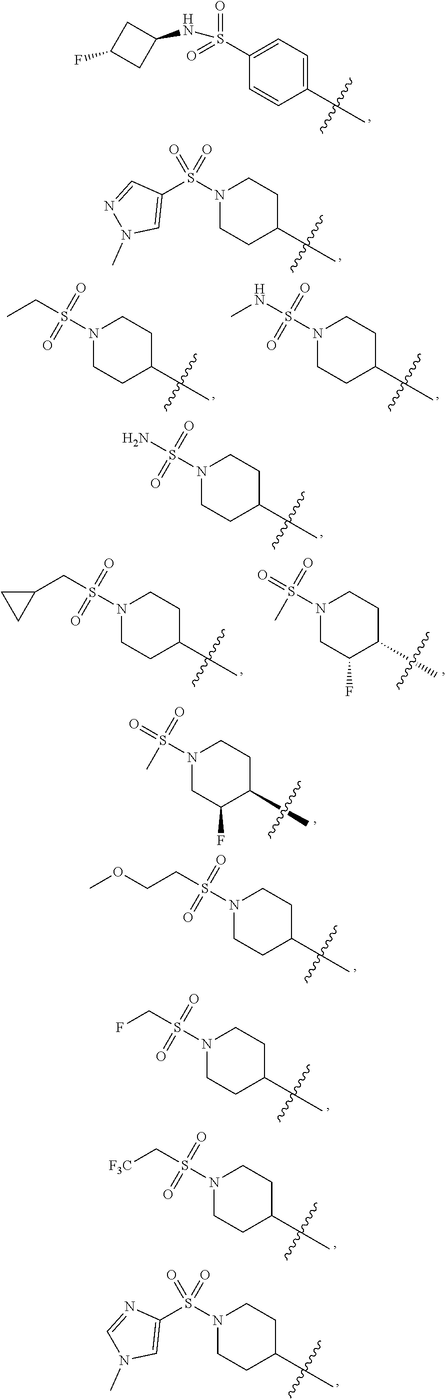

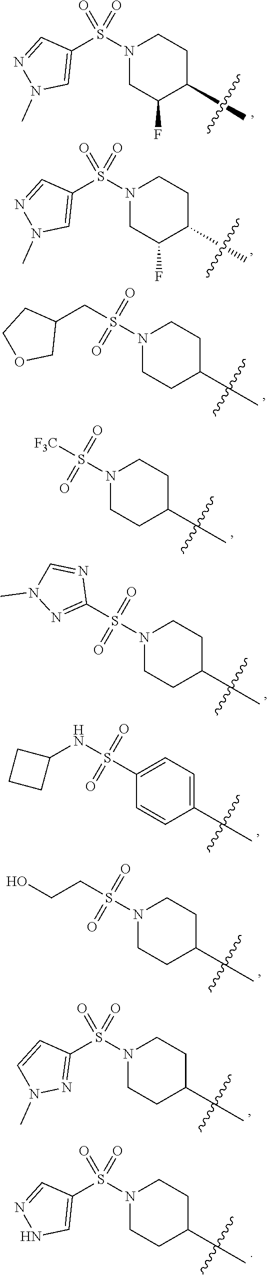

In some embodiments, L¹-R¹ in Formula I can be

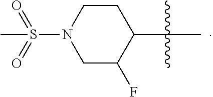 [SMILES: *C1CCN(S(C)(=O)=O)CC1F]

In some embodiments, L¹-R¹ in Formula I can be

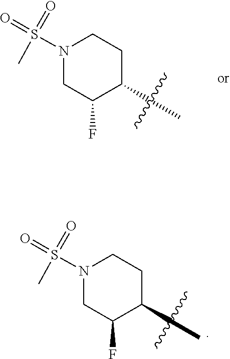 [SMILES: *[C@H]1CCN(S(C)(=O)=O)C[C@H]1F ; *[C@@H]1CCN(S(C)(=O)=O)C[C@@H]1F]

In some preferred embodiments, L¹-R¹ in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, I-A-5A, I-A-6A, I-A-7A, I-A-8A, I-A-9A, I-A-10A, or I-B) can be selected from:

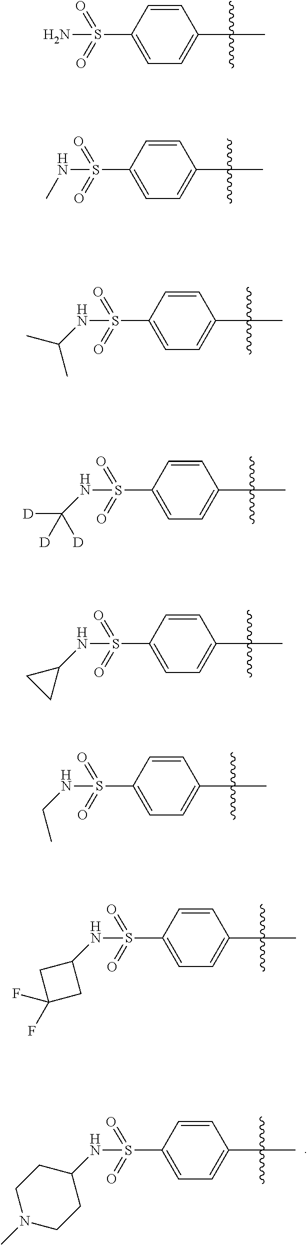

In some preferred embodiments, L¹-R¹ in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, I-A-5B, I-A-6B, I-A-7B, I-A-8B, I-A-9B, I-A-10B, or I-B) as applicable can contain a piperidine ring, such as

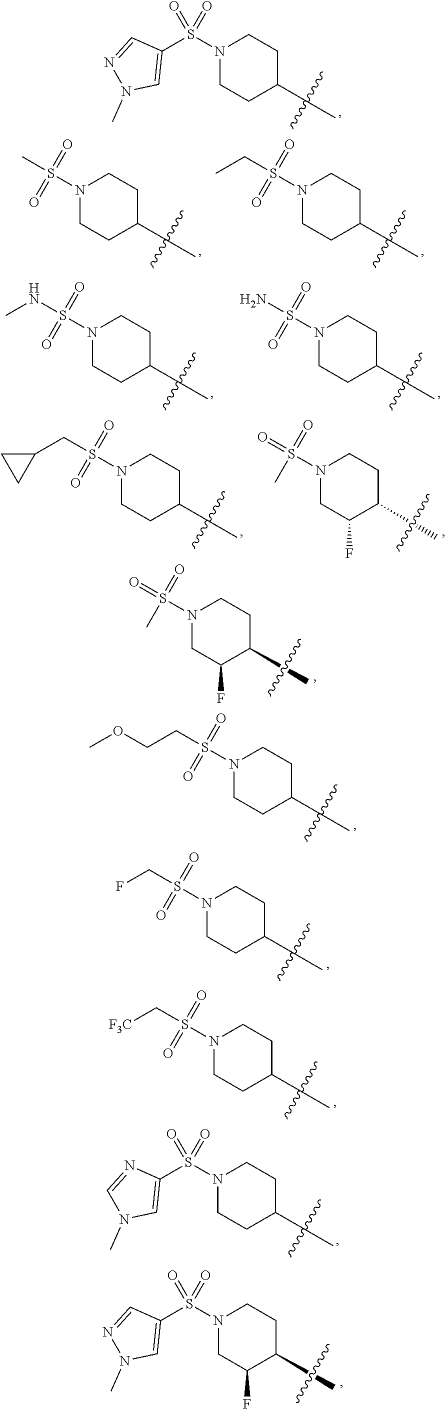

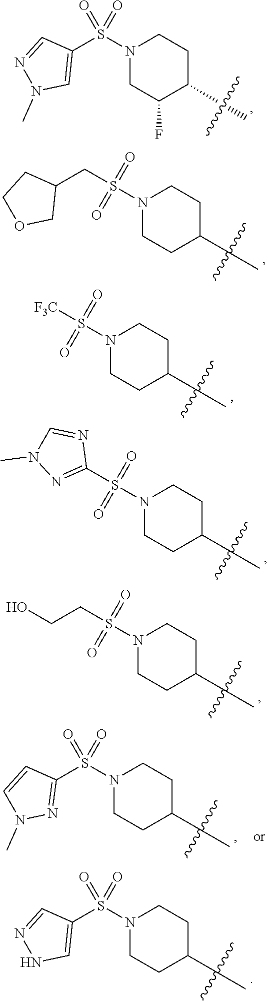

For example, in some embodiments, the compound of Formula I-A can be characterized as having a formula according to any of the following Formula I-A-2, I-A-2, I-A-3, or I-A-4:

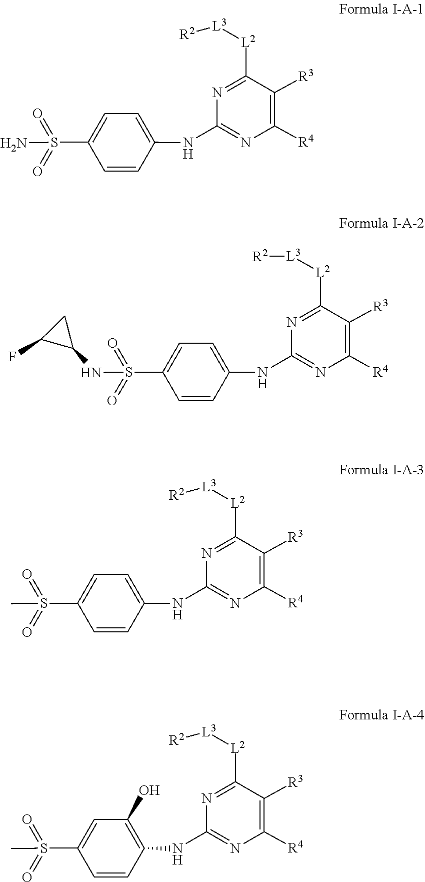

wherein L², L³, R², R³, and R⁴ include any of those described herein in any combination.

In some embodiments, L² in Formula I (e.g., Formula I-A, I-A-1, I-A-2, I-A-3, I-A-4, I-A-5A, I-A-6A, I-A-7A, I-A-8A, I-A-9A, I-A-10A, I-A-51B, I-A-61B, I-A-71B, I-A-8B, I-A-9B, I-A-10B, or I-B) can be a bond, in which case, L³-R² is directly attached to the pyridine or pyrimidine ring in Formula I.

In some embodiments, L² in Formula I (e.g., Formula I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B3, I-A-8B, I-A-9B, I-A-10B, or I-B) can be —O—.

In some embodiments, L² in Formula I (e.g., Formula I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B, I-A-8B, I-A-9B, I-A-10B, or I-B) can be —N(R¹⁴)—, wherein R¹⁴ is defined herein. For example, in some embodiments, R¹⁴ can be hydrogen. In some embodiments, R¹⁴ can be a C₁₋₄ alkyl optionally substituted with oxo, F, CN, G¹, OH, O-G¹, NH₂, NH(G¹), and N(G¹)(G¹), wherein G¹ at each occurrence is independently a C₁₋₄ alkyl optionally substituted with 1-3 substituents independently selected from F, CN, OH, and C₁₋₄heteroalkyl, or a C₃₋₆ cycloalkyl optionally substituted with 1-3 substituents independently selected from F, CN, OH, and C₁₋₄ heteroalkyl.

In some embodiments, L³ in Formula I (e.g., Formula I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B, I-A-8B, I-A-9B, I-A-10B, or I-B) can be a bond, in which case, R² is directly attaching to L², or if L² is also a bond, then R² is directly attached to the pyridine or pyrimidine ring in Formula I.

In some embodiments, L³ in Formula I (e.g., Formula I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B, I-A-8B, I-A-9B, I-A-10B, or I-B) can be an optionally substituted C₁₋₄ alkylene, such as CH₂.

In some embodiments, L³ in Formula I (e.g., Formula I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B, I-A-8B, I-A-9B, I-A-10B, or I-B) can be an optionally substituted C₁₋₄ heteroalkylene, e.g., as described herein.

Various groups are suitable for use as R² in Formula I. For example, in some embodiments, R² can be hydrogen. In some embodiments, R² can be an optionally substituted C₃-s alkyl. In some embodiments, R² can be an optionally substituted C₃₋₈ carbocyclyl. In some embodiments, R² can be an optionally substituted 4-10 membered heterocyclyl, e.g., monocyclic or bicyclic (e.g., fused, bridged, or spiro bicyclic) heterocyclyl having 1 or 2 ring heteroatoms independently selected from N, O, and S. In some embodiments, R² can be an optionally substituted phenyl. In some embodiments, R² can be an optionally substituted 5-10 membered heteroaryl, such as a 5 or 6 membered heteroaryl having 1-3 ring heteroatoms independently selected from N, O, and S.

In some embodiments, in Formula I (e.g., Formula I-1, I-2, I-3, I-4, I-5, I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B, I-A-8B, I-A-9B, I-A-10B, or I-B), R² can be a C₃₋₈ alkyl substituted with one or more (e.g., 1, 2, or 3) substituents independently selected from oxo, F, G¹, CN, OH, O-G¹, NH₂, NH(G¹), and N(G¹)(G¹), wherein G¹ at each occurrence is independently a C₁₋₄ alkyl optionally substituted with 1-3 substituents independently selected from F, CN, OH, and C₁₋₄ heteroalkyl or a C₃₋₆ cycloalkyl optionally substituted with 1-3 substituents independently selected from F, CN, OH, and C₁₋₄heteroalkyl. In any of the embodiments herein, unless specified or otherwise contrary from context, in Formula I (e.g., Formula I-1, I-2, I-3, I-4, I-5, I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B, I-A-8B, I-A-9B, I-A-10B, or I-B), R² can be selected from:

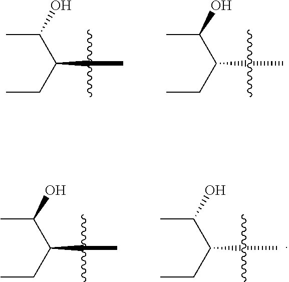

In any of the embodiments herein, unless specified or otherwise contrary from context, in Formula I (e.g., Formula I-1, I-2, I-3, I-4, I-5, I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B, I-A-8B, I-A-9B, I-A-10B, or I-B), R² can be selected from:

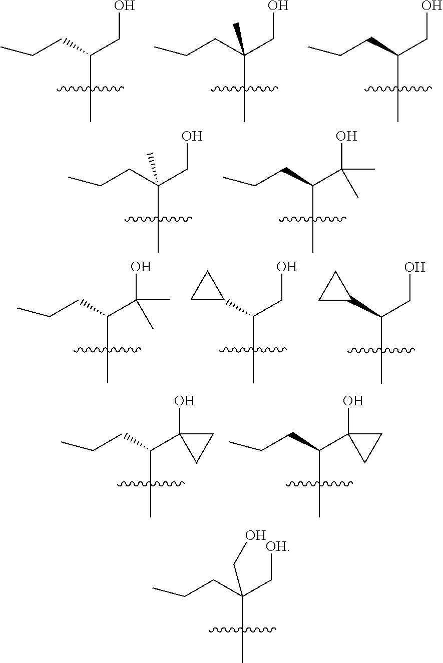

In some embodiments, in Formula I (e.g., Formula I-1, I-2, I-3, I-4, I-5, I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B, I-A-8B, I-A-9B, I-A-10B, or I-B), R² can be a C₃₋₈ cycloalkyl optionally substituted with one or more (e.g., 1, 2, or 3) substituents independently selected from F, CN, G¹, OH, COOH, C(O)-G¹, O-G¹, C(O)—O-G¹, NH₂, NH(G¹), N(G¹)(G¹), C(O)—NH₂, C(O)—NH(G¹), C(O)—N(G¹)(G¹), wherein G¹ at each occurrence is independently a C₁₋₄ alkyl optionally substituted with 1-3 substituents independently selected from F, CN, OH, and C₁₋₄ heteroalkyl, or a C₃₋₆ cycloalkyl optionally substituted with 1-3 substituents independently selected from F, CN, OH, and C₁₋₄ heteroalkyl.

In some preferred embodiments, in Formula I (e.g., Formula I-1, I-2, I-3, I-4, I-5, I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B, I-A-8B, I-A-9B, I-A-10B, or I-B), R² is a C₃₋₆ cycloalkyl, such as cyclopropyl, cyclobutyl, cyclopentyl, cyclohexyl, which is optionally substituted with one or more (e.g., 1, 2, or 3) substituents independently selected from F, methyl, ethyl, hydroxyethyl (e.g., —CH₂CH₂OH or —CH(OH)CH₃), —C(O)CH₃, OH, —CH₂OH, fluorine substituted methyl (e.g., —CF₂H), and fluorine substituted ethyl (e.g., —CH₂CF₂H). In some embodiments, in Formula I (e.g., Formula I-1, I-2, I-3, I-4, I-5, I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B, I-A-8B, I-A-9B, I-A-10B, or I-B), R² is a C₃₋₆ cycloalkyl, such as cyclopropyl, cyclobutyl, cyclopentyl, cyclohexyl, which is substituted with one or two substituents independently selected from OH, —CH₂CH₂OH, —CH(OH)CH₃), —CH₂OH, —CF₂H, and —CH₂CF₂H, and optionally further substituted with F, methyl, or ethyl.

In some preferred embodiments, in Formula I (e.g., Formula I-1, I-2, I-3, I-4, I-5, I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B, I-A-8B, I-A-9B, I-A-10B, or I-B), R² is a spiro, fused, or bridged C₆-s cycloalkyl, such as

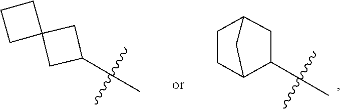 [SMILES: *C1CC2(CCC2)C1 ; *C1CC2CCC1C2]

which is optionally substituted with one or more (e.g., 1, 2, or 3) substituents independently selected from F, methyl, ethyl, hydroxyethyl (e.g., —CH₂CH₂OH or —CH(OH)CH₃), —C(O)CH₃, OH, —CH₂OH, fluorine substituted methyl (e.g., —CF₂H), and fluorine substituted ethyl (e.g., —CH₂CF₂H). In some embodiments, in Formula I (e.g., Formula I-1, I-2, I-3, I-4, I-5, I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B, I-A-8B, I-A-9B, I-A-10B, or I-B), R² is a spiro, fused, or bridged C₆-s cycloalkyl, such as

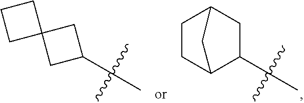 [SMILES: *C1CC2(CCC2)C1 ; *C1CC2CCC1C2]

which is substituted with one or two substituents independently selected from OH, —CH₂CH₂OH, —CH(OH)CH₃), —CH₂OH, —CF₂H, and —CH₂CF₂H, and optionally further substituted with F, methyl, or ethyl.

In some embodiments, in Formula I (e.g., Formula I-1, I-2, I-3, I-4, I-5, I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B, I-A-8B, I-A-9B, I-A-10B, or I-B), R² can be a 4-10 membered heterocyclyl having 1-4 ring heteroatoms independently selected from N, O, and S, which is optionally substituted with one or more (e.g., 1, 2, or 3) substituents independently selected from oxo, F, CN, G¹, OH, COOH, C(O)-G¹, O-G¹, C(O)—O-G¹, NH₂, NH(G¹), N(G¹)(G¹), C(O)—NH₂, C(O)—NH(G¹), C(O)—N(G¹)(G¹), G², O-G², NH(G²), N(G¹)(G²), C(O)—NH(G²), and C(O)—N(G¹)(G²), wherein G¹ at each occurrence is independently a C₁₋₄ alkyl optionally substituted with 1-3 substituents independently selected from F, CN, OH, and C₁₋₄ heteroalkyl, or a C₃₋₆ cycloalkyl optionally substituted with 1-3 substituents independently selected from F, CN, OH, and C₁₋₄ heteroalkyl; wherein G² at each occurrence is independently a 4-6 membered heterocyclyl having 1-2 ring heteroatoms independently selected from N, O, and S, phenyl or 5- or 6-membered heteroaryl, each of which is optionally substituted with 1-3 substituents independently selected from oxo (as applicable), F, CN, G¹, OH, O-G¹, NH₂, NH(G¹), and N(G¹)(G¹); and wherein two substituents of the 4-10 membered heterocyclyl, together with the intervening atom(s), can optionally be joined to form a fused, bridged, or spiro ring structure.

In some embodiments, in Formula I (e.g., Formula I-1, I-2, I-3, I-4, I-5, I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B, I-A-8B, I-A-9B, I-A-10B, or I-B), R² is a 4-8 membered heterocyclyl having 1-2 ring heteroatoms independently selected from N, O, and S, which is optionally substituted with one or more (e.g., 1, 2, or 3) substituents independently selected from oxo, F, CN, G¹, OH, COOH, C(O)-G¹, O-G¹, C(O)—O-G¹, NH₂, NH(G¹), N(G¹)(G¹), C(O)—NH₂, C(O)—NH(G¹), C(O)—N(G¹)(G¹), wherein G¹ at each occurrence is independently a C₁₋₄ alkyl optionally substituted with 1-3 substituents independently selected from F, CN, OH, and C₁₋₄ heteroalkyl, or a C₃₋₆ cycloalkyl optionally substituted with 1-3 substituents independently selected from F, CN, OH, and C₁₋₄heteroalkyl.

In some embodiments, in Formula I (e.g., Formula I-1, I-2, I-3, I-4, I-5, I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B, I-A-8B, I-A-9B, I-A-10B, or I-B), R² can be a 4-8 membered monocyclic, saturated or partially unsaturated, heterocyclyl having 1-2 ring heteroatoms independently selected from N, O, and S, such as pyrrolidine, piperidine, azepane, etc., which is optionally substituted with one or more (e.g., 1, 2, or 3) substituents independently selected from oxo, F, CN, G¹, OH, COOH, C(O)-G¹, O-G¹, C(O)—O-G¹, NH₂, NH(G¹), N(G¹)(G¹), C(O)—NH₂, C(O)—NH(G¹), C(O)—N(G¹)(G¹), G², O-G², NH(G²), N(G¹)(G²), C(O)—NH(G²), and C(O)—N(G¹)(G²), wherein G¹ at each occurrence is independently a C₁₋₄ alkyl optionally substituted with 1-3 substituents independently selected from F, CN, OH, and C₁₋₄ heteroalkyl, or a C₃₋₆ cycloalkyl optionally substituted with 1-3 substituents independently selected from F, CN, OH, and C₁₋₄ heteroalkyl; wherein G² at each occurrence is independently a 4-6 membered heterocyclyl having 1-2 ring heteroatoms independently selected from N, O, and S, phenyl or 5- or 6-membered heteroaryl, each of which is optionally substituted with 1-3 substituents independently selected from oxo (as applicable), F, CN, G¹, OH, O-G¹, NH₂, NH(G¹), and N(G¹)(G¹); and wherein two substituents of the 4-8 membered heterocyclyl, together with the intervening atom(s), can optionally be joined to form a fused, bridged, or spiro ring structure.

In some embodiments, in Formula I (e.g., Formula I-1, I-2, I-3, I-4, I-5, I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B, I-A-8B, I-A-9B, I-A-10B, or I-B), R² can be a 4-6 or 7 membered monocyclic heterocyclyl having 1-2 ring heteroatoms independently selected from N, O, and S, such as oxetane, azetidine, tetrahydrofuran, tetrahydropyran, oxepane, pyrrolidine, piperidine, etc., which is optionally substituted with one or more (e.g., 1, 2, or 3) substituents independently selected from oxo, F, methyl, ethyl, hydroxyethyl (e.g., —CH₂CH₂OH or —CH(OH)CH₃), —C(O)CH₃, OH, —CH₂OH, fluorine substituted methyl (e.g., —CF₂H), and fluorine substituted ethyl (e.g., —CH₂CF₂H). In some embodiments, in Formula I (e.g., Formula I-1, I-2, I-3, I-4, I-5, I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B, I-A-8B, I-A-9B, I-A-10B, or I-B), R² can be a 4-6 or 7 membered monocyclic heterocyclyl having 1-2 ring heteroatoms independently selected from N, O, and S, such as oxetane, azetidine, tetrahydrofuran, tetrahydropyran, oxepane, pyrrolidine, piperidine, etc., which is substituted with one or two substituents independently selected from OH, —CH₂CH₂OH, —CH(OH)CH₃), —CH₂OH, —CF₂H, and —CH₂CF₂H, and optionally further substituted with F, methyl, or ethyl.

In some embodiments, in Formula I (e.g., Formula I-1, I-2, I-3, I-4, I-5, I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B, I-A-8B, I-A-9B, I-A-10B, or I-B), R² can be selected from:

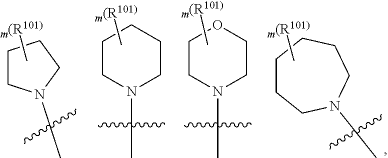

wherein:
m is 0, 1, 2, 3, or 4;
R¹⁰¹ at each occurrence is independently oxo, F, CN, G¹, G², OH, O-G¹, and O-G², wherein G¹ at each occurrence is independently a C₁₋₄ alkyl optionally substituted with 1-3 substituents independently selected from F, CN, OH, and C₁₋₄ heteroalkyl, or a C₃₋₆ cycloalkyl optionally substituted with 1-3 substituents independently selected from F, CN, OH, and C₁₋₄ heteroalkyl; wherein G² at each occurrence is independently 4-6 membered heterocyclyl having 1-2 ring heteroatoms independently selected from N, O, and S, phenyl or 5- or 6-membered heteroaryl having 1-4 ring heteroatoms independently selected from N, O, and S, each of which is optionally substituted with 1-3 substituents independently selected from F, CN, G¹, OH, and O-G¹; wherein two R¹⁰¹, together with the intervening atom(s), can optionally be joined to form a fused, bridged, or spiro ring structure. In some embodiments, m can be 0, 1, 2, or 3. For example, in some embodiments, m is 0, i.e., the heterocyclyl is not substituted. In some embodiments, m is 1. In some embodiments, m is 2. In some embodiments, R¹⁰¹ at each occurrence is independently F, OH, CN, C₁₋₄ alkyl (e.g., methyl, ethyl, propyl, etc.) phenyl, cyclopropyl, hydroxymethyl (—CH₂OH), methoxy, fluorine substituted methoxy, fluorine substituted C₁₋₄ alkyl, such as fluorine substituted methyl such as CF₂H, or fluorine substituted ethyl (e.g., CH₂CF₂H).

In some preferred embodiments, in Formula I (e.g., Formula I-1, I-2, I-3, I-4, I-5, I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B, I-A-8B, I-A-9B, I-A-10B, or I-B), R² can be selected from:

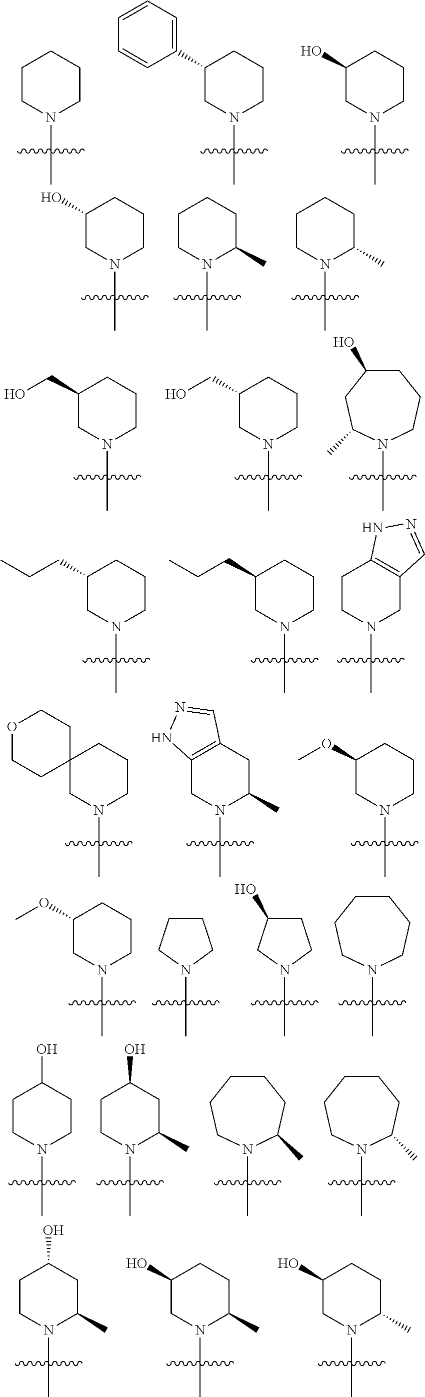

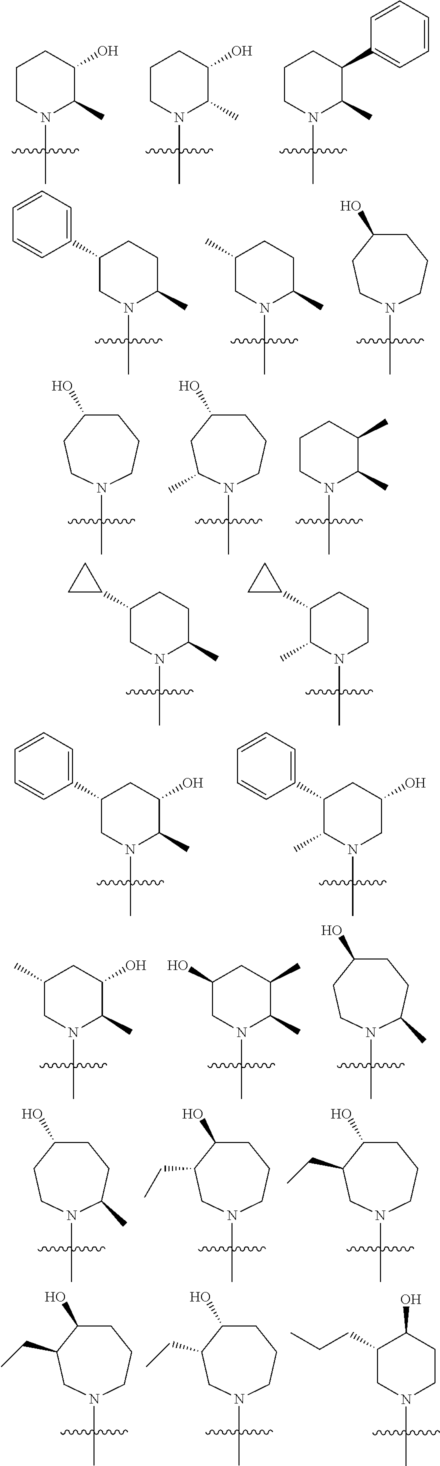

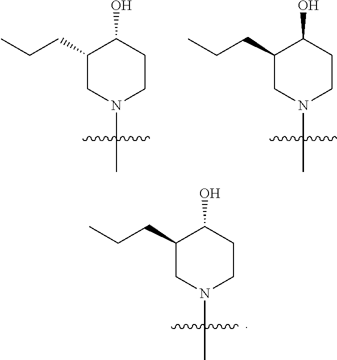

In some embodiments, in Formula I (e.g., Formula I-1, I-2, I-3, I-4, I-5, I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B, I-A-8B, I-A-9B, I-A-10B, or I-B), R² can also be a phenyl optionally substituted with one or more (e.g., 1, 2, or 3) substituents independently selected from F, CN, G¹, OH, COOH, C(O)-G¹, O-G¹, C(O)—O-G¹, NH₂, NH(G¹), N(G¹)(G¹), C(O)—NH₂, C(O)—NH(G¹), C(O)—N(G¹)(G¹), G², O-G², NH(G²), N(G¹)(G²), C(O)—NH(G²), and C(O)—N(G¹)(G²), wherein G¹ at each occurrence is independently a C₁₋₄ alkyl optionally substituted with 1-3 substituents independently selected from F, CN, OH, and C₁₋₄heteroalkyl, or a C₃₋₆ cycloalkyl optionally substituted with 1-3 substituents independently selected from F, CN, OH, and C₁₋₄ heteroalkyl; wherein G² at each occurrence is independently a 4-6 membered heterocyclyl having 1-2 ring heteroatoms independently selected from N, O, and S, phenyl or 5- or 6-membered heteroaryl, each of which is optionally substituted with 1-3 substituents independently selected from oxo (as applicable), F, CN, G¹, OH, O-G¹, NH₂, NH(G¹), and N(G¹)(G¹); wherein two optional substituents of the phenyl group, together with the intervening atom(s), can optionally be joined to form a fused ring structure.

For example, in some embodiments, in Formula I (e.g., Formula I-1, I-2, I-3, I-4, I-5, I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B, I-A-8B, I-A-9B, I-A-10B, or I-B), R² can be

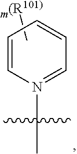

wherein:
m is 0, 1, 2, or 3;
R¹⁰¹ at each occurrence is independently F, CN, G¹, G², OH, O-G¹, O-G², NH₂, NH(G¹), NH(G²), N(G¹)(G¹), and N(G¹)(G²), wherein G¹ at each occurrence is independently a C₁₋₄ alkyl optionally substituted with 1-3 substituents independently selected from F, OH, and C₁₋₄ heteroalkyl or a C₃₋₆ cycloalkyl optionally substituted with 1-3 substituents independently selected from F, OH, and C₁₋₄heteroalkyl; wherein G² at each occurrence is independently 4-6 membered heterocyclyl having 1-2 ring heteroatoms independently selected from N, O, and S, phenyl or 5- or 6-membered heteroaryl having 1-4 ring heteroatoms independently selected from N, O, and S, each of which is optionally substituted with 1-3 substituents independently selected from F, CN, G¹, OH, and O-G¹; wherein two R¹⁰¹, together with the intervening atom(s), can optionally be joined to form a fused ring structure. In some embodiments, m can be 0, 1, 2, or 3. For example, in some embodiments, m is 0, i.e., the phenyl is not substituted. In some embodiments, m is 1. In some embodiments, m is 2. In some embodiments, m is 3. In some embodiments, R¹⁰¹ at each occurrence is independently F, OH, CN, C₁₋₄ alkyl (e.g., methyl, ethyl, propyl, etc.), cyclopropyl, cyclobutyl, oxetanyl, C₁₋₄ alkoxy (e.g., methoxy), fluorine substituted C₁₋₄ alkoxy such as fluorine substituted methoxy, fluorine substituted C₁₋₄ alkyl, such as fluorine substituted methyl such as CF₂H, or fluorine substituted ethyl (e.g., CH₂CF₂H). In some preferred embodiments, R¹⁰¹ at each occurrence is independently F, C₁₋₄ alkyl (e.g., methyl, ethyl, n-propyl, etc.), OH, cyclopropyl, cyclobutyl, oxetanyl, or CN.

In some preferred embodiments, in Formula I (e.g., Formula I-1, I-2, I-3, I-4, I-5, I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B, I-A-8B, I-A-9B, I-A-10B, or I-B), R² can be selected from:

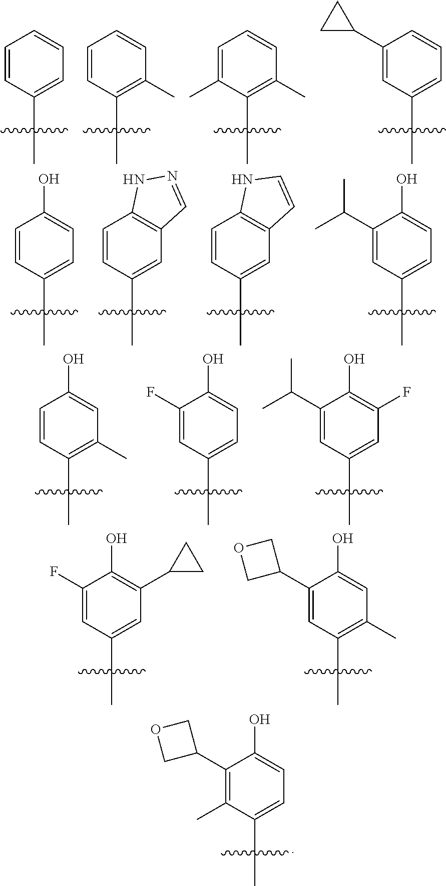

In some embodiments, in Formula I (e.g., Formula I-1, I-2, I-3, I-4, I-5, I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B, I-A-8B, I-A-9B, I-A-10B, or I-B), R² can also be a 5-10 membered heteroaryl having 1-4 ring heteroatoms independently selected from N, O, and S, which is optionally substituted with one or more (e.g., 1, 2, or 3) substituents independently selected from F, CN, G¹, OH, COOH, C(O)-G¹, O-G¹, C(O)—O-G¹, NH₂, NH(G¹), N(G¹)(G¹), C(O)—NH₂, C(O)—NH(G¹), C(O)—N(G¹)(G¹), G², O-G², NH(G²), N(G¹)(G²), C(O)—NH(G²), and C(O)—N(G¹)(G²), wherein G¹ at each occurrence is independently a C₁₋₄ alkyl optionally substituted with 1-3 substituents independently selected from F, CN, OH, and C₁₋₄ heteroalkyl, or a C₃₋₆ cycloalkyl optionally substituted with 1-3 substituents independently selected from F, CN, OH, and C₁₋₄ heteroalkyl; wherein G² at each occurrence is independently a 4-6 membered heterocyclyl having 1-2 ring heteroatoms independently selected from N, O, and S, phenyl or 5- or 6-membered heteroaryl, each of which is optionally substituted with 1-3 substituents independently selected from oxo (as applicable), F, CN, G¹, OH, O-G¹, NH₂, NH(G¹), and N(G¹)(G¹); and wherein two optional substituents of the heteroaryl group, together with the intervening atom(s), can optionally be joined to form a fused ring structure.

In some embodiments, in Formula I (e.g., Formula I-1, I-2, I-3, I-4, I-5, I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B, I-A-8B, I-A-9B, I-A-10B, or I-B), R² can be a 5- or 6-membered heteroaryl having 1-4 ring heteroatoms independently selected from N, O, and S, such as pyridyl (e.g., 2-, 3-, or 4-pyridyl), pyrazole, etc., which is optionally substituted with one or more (e.g., 1, 2, or 3) substituents independently selected from F, CN, G¹, OH, COOH, C(O)-G¹, O-G¹, C(O)—O-G¹, NH₂, NH(G¹), N(G¹)(G¹), C(O)—NH₂, C(O)—NH(G¹), C(O)—N(G¹)(G¹), G², O-G², NH(G²), N(G¹)(G²), C(O)—NH(G²), and C(O)—N(G¹)(G²), wherein G¹ at each occurrence is independently a C₁₋₄ alkyl optionally substituted with 1-3 substituents independently selected from F, CN, OH, and C₁₋₄ heteroalkyl, or a C₃₋₆ cycloalkyl optionally substituted with 1-3 substituents independently selected from F, CN, OH, and C₁₋₄ heteroalkyl; wherein G² at each occurrence is independently a 4-6 membered heterocyclyl having 1-2 ring heteroatoms independently selected from N, O, and S, phenyl or 5- or 6-membered heteroaryl, each of which is optionally substituted with 1-3 substituents independently selected from oxo (as applicable), F, CN, G¹, OH, O-G¹, NH₂, NH(G¹), and N(G¹)(G¹); and wherein two optional substituents of the heteroaryl group, together with the intervening atom(s), can optionally be joined to form a fused ring structure. In any of the embodiments herein, unless specified or otherwise contrary from context, in Formula I (e.g., Formula I-1, I-2, I-3, I-4, I-5, I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B, I-A-8B, I-A-9B, I-A-10B, or I-B), R² can be selected from:

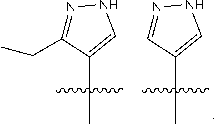

In some embodiments, in Formula I (e.g., Formula I-1, I-2, I-3, I-4, I-5, I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B, I-A-8B, I-A-9B, I-A-10B, or I-B), R² can be a 8-10-membered bicyclic heteroaryl having 1-4 ring heteroatoms independently selected from N, O, and S, such as indolyl, indazolyl, etc., which is optionally substituted with one or more (e.g., 1, 2, or 3) substituents independently selected from F, CN, G¹, OH, COOH, C(O)-G¹, O-G¹, C(O)—O-G¹, NH₂, NH(G¹), N(G¹)(G¹), C(O)—NH₂, C(O)—NH(G¹), C(O)—N(G¹)(G¹), G², O-G², NH(G²), N(G¹)(G²), C(O)—NH(G²), and C(O)—N(G¹)(G²), wherein G¹ at each occurrence is independently a C₁₋₄ alkyl optionally substituted with 1-3 substituents independently selected from F, CN, OH, and C₁₋₄ heteroalkyl, or a C₃₋₆ cycloalkyl optionally substituted with 1-3 substituents independently selected from F, CN, OH, and C₁₋₄ heteroalkyl; wherein G² at each occurrence is independently a 4-6 membered heterocyclyl having 1-2 ring heteroatoms independently selected from N, O, and S, phenyl or 5- or 6-membered heteroaryl, each of which is optionally substituted with 1-3 substituents independently selected from oxo (as applicable), F, CN, G¹, OH, O-G¹, NH₂, NH(G¹), and N(G¹)(G¹); and wherein two optional substituents of the heteroaryl group, together with the intervening atom(s), can optionally be joined to form a fused ring structure.

In any of the embodiments herein, unless specified or otherwise contrary from context, in Formula I (e.g., Formula I-1, I-2, I-3, I-4, I-5, I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B, I-A-8B, I-A-9B, I-A-10B, or I-B), R² can be selected from:

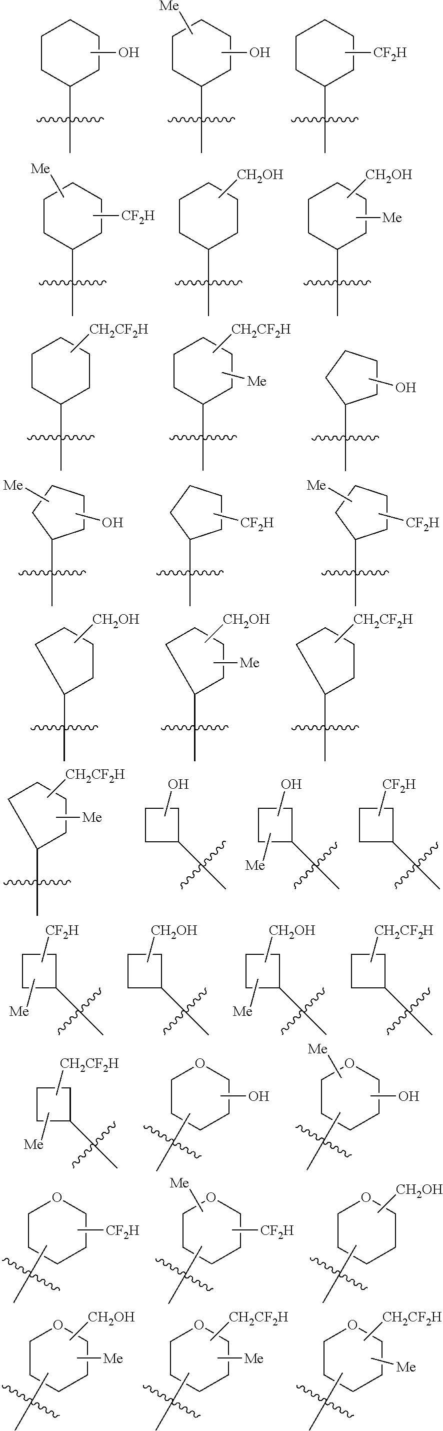

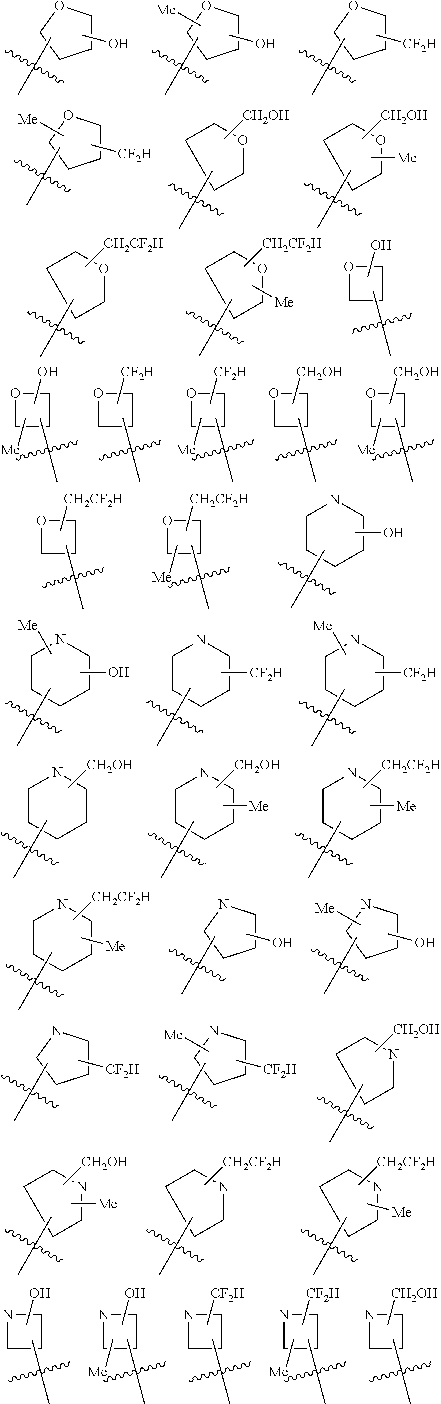

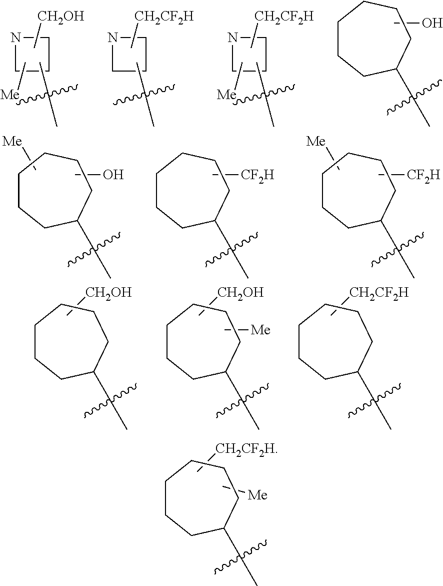

In any of the embodiments herein, unless specified or otherwise contrary from context, in Formula I (e.g., Formula I-1, I-2, I-3, I-4, I-5, I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B, I-A-8B, I-A-9B, I-A-10B, or I-B), R² can be selected from:

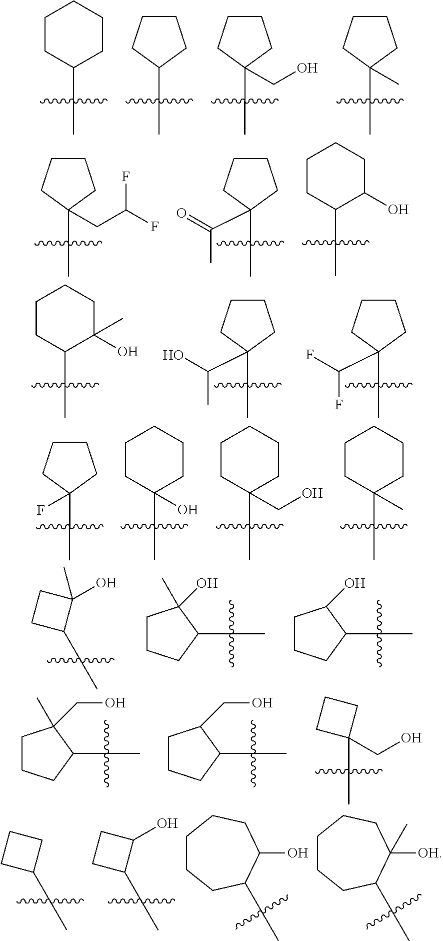

In any of the embodiments herein, unless specified or otherwise contrary from context, in Formula I (e.g., Formula I-1, I-2, I-3, I-4, I-5, I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B, I-A-8B, I-A-9B, I-A-10B, or I-B), R² can be selected from:

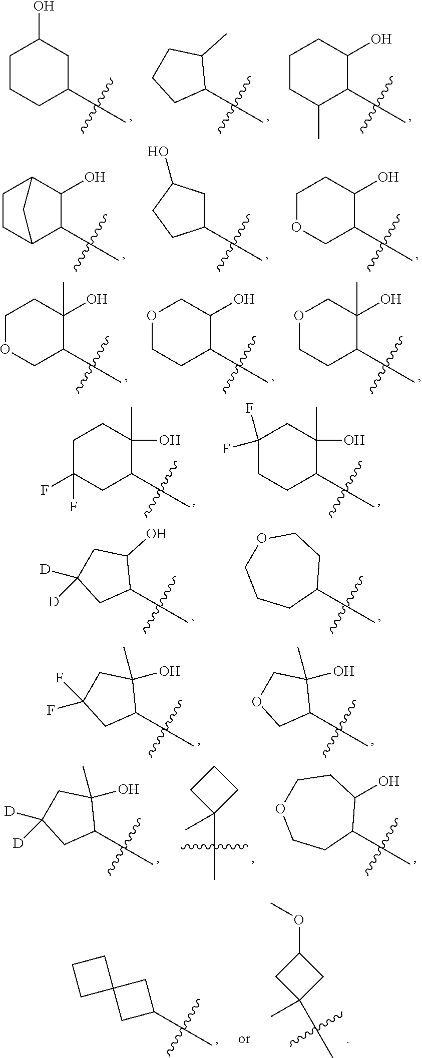

In any of the embodiments herein, unless specified or otherwise contrary from context, in Formula I (e.g., Formula I-1, I-2, I-3, I-4, I-5, I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B, I-A-8B, I-A-9B, I-A-10B, or I-B), R² can be selected from:

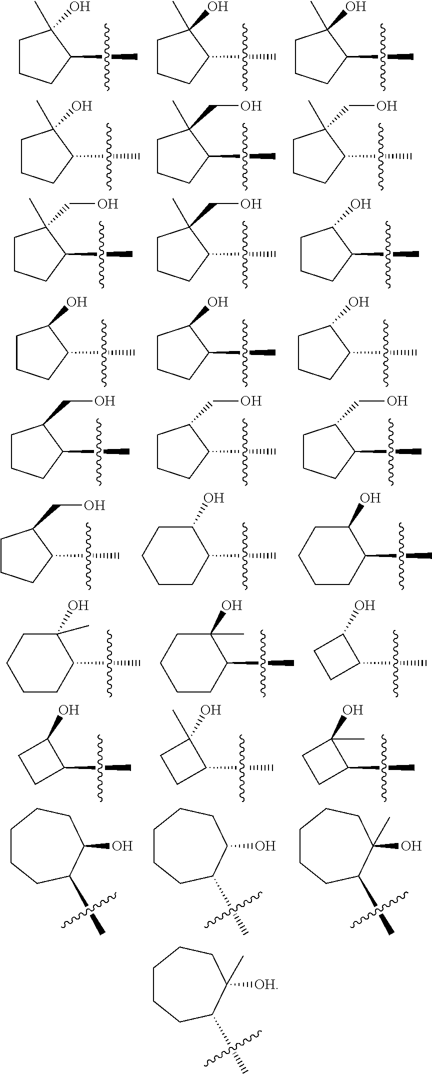

In any of the embodiments herein, unless specified or otherwise contrary from context, in Formula I (e.g., Formula I-1, I-2, I-3, I-4, I-5, I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B, I-A-8B, I-A-9B, I-A-10B, or I-B), R² can be selected from:

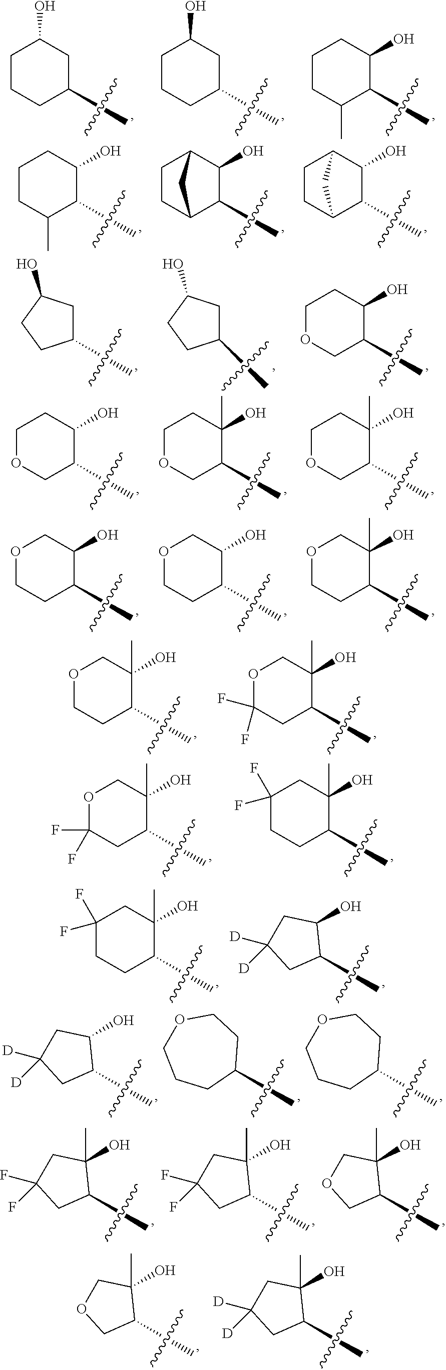

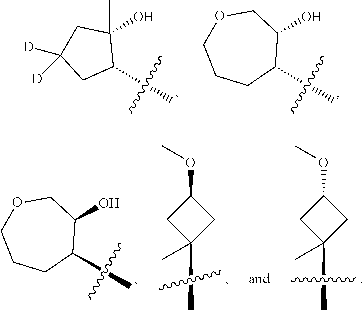

In any of the embodiments herein, unless specified or otherwise contrary from context, in Formula I (e.g., Formula I-1, I-2, I-3, I-4, I-5, I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B, I-A-8B, I-A-91, I-A-10B, or I-B), R² can be selected from:

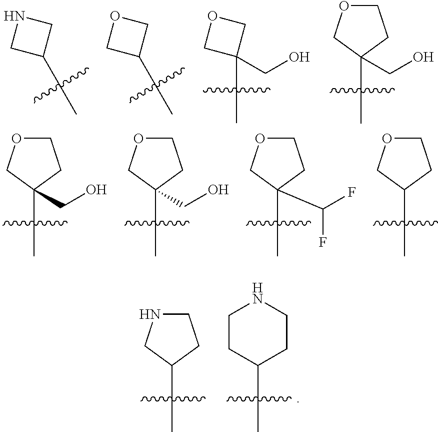

Combinations of R², L² and L³ in Formula I are not particularly limited. For example, in some embodiments, in Formula I (e.g., Formula I-A, I-A-1, I-A-2, I-A-3, I-A-4, I-A-5A, I-A-6A, I-A-7A, I-A-8A, I-A-9A, I-A-10A, I-A-51B, I-A-6B, I-A-7B, I-A-8B, I-A-9B, I-A-10B, or I-B), L² can be —O— and L³ can be a bond or a C₁₋₄ alkylene (e.g., CH₂) optionally substituted with one or more (e.g., 1, 2, or 3) substituents independently selected from F, OH, and protected OH. For example, in some embodiments, the compound of Formula I (e.g., Formula I-A, I-A-1, I-A-2, I-A-3, I-A-4, I-A-5, I-A-6, I-A-7, I-A-8, I-A-9, or I-A-10) can be characterized as having Formula I-1 or I-2:

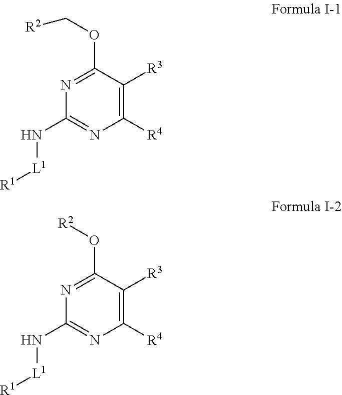

wherein L¹, R¹, R², R³, and R⁴ include any of those described herein in any combination.

In some embodiments, in Formula I (e.g., Formula I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B, I-A-8B, I-A-9B, I-A-10B, or I-B), L² can be —N(R¹⁴)—, wherein R¹⁴ is defined herein, and L³ can be a bond or a C₁₋₄ alkylene (e.g., CH₂) optionally substituted with one or more (e.g., 1, 2, or 3) substituents independently selected from F, OH, and protected OH. For example, in some embodiments, the compound of Formula I (e.g., Formula I-A, I-A-1, I-A-2, I-A-3, I-A-4, I-A-5, I-A-6, I-A-7, I-A-8, I-A-9, or I-A-10) can be characterized as having Formula I-3 or I-4:

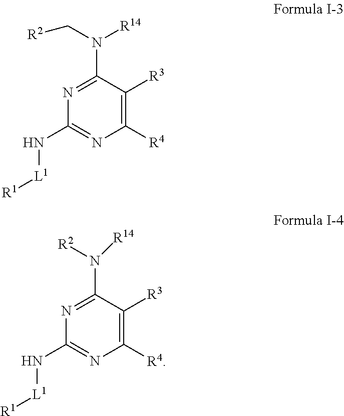

wherein L¹, R¹, R², R³, R⁴ and R¹⁴ include any of those described herein in any combination. Typically, R¹⁴ in Formula I-3 or I-4 is hydrogen or a C₁₋₄ alkyl (e.g., methyl).

In some preferred embodiments, the compound of Formula I (e.g., Formula I-A, I-A-1, I-A-2, I-A-3, I-A-4, I-A-5, I-A-6, I-A-7, I-A-8, I-A-9, or I-A-10) can be characterized as having Formula I-1, I-2, I-3 or I-4, wherein R² is a C₃₋₈ alkyl substituted with one or more (e.g., 1, 2, or 3) substituents independently selected from oxo, F, G¹, CN, OH, O-G¹, NH₂, NH(G¹), and N(G¹)(G¹), wherein G¹ at each occurrence is independently a C₁₋₄ alkyl optionally substituted with 1-3 substituents independently selected from F, CN, OH, and C₁₋₄ heteroalkyl or a C₃₋₆ cycloalkyl optionally substituted with 1-3 substituents independently selected from F, CN, OH, and C₁₋₄ heteroalkyl; wherein two optional substituents of the C₃₋₈ alkyl, together with the intervening atom(s), can optionally be joined to form a ring structure, such as a spiro-C₃₋₆ cycloalkyl or 4-7 membered heterocyclyl. In any of the embodiments herein, unless specified or otherwise contrary from context, in Formula I-1, I-2, I-3 or I-4, R² can be selected from the following

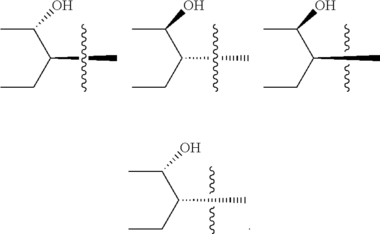

In any of the embodiments herein, unless specified or otherwise contrary from context, in Formula I-1, I-2, I-3 or I-4, R² can be selected from the following:

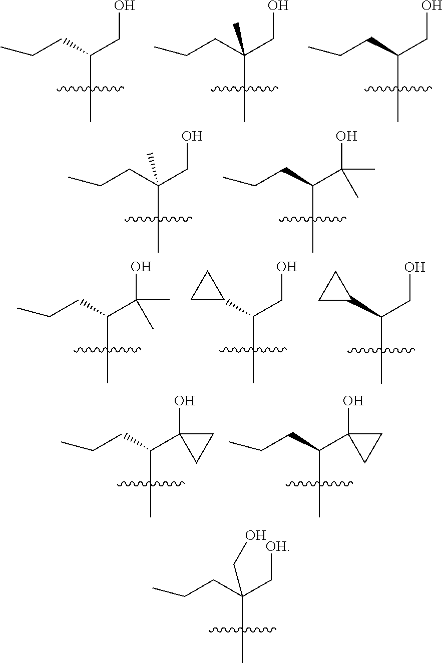

In some preferred embodiments, the compound of Formula I (e.g., Formula I-A, I-A-1, I-A-2, I-A-3, I-A-4, I-A-5, I-A-6, I-A-7, I-A-8, I-A-9, or I-A-10) can be characterized as having Formula I-1, I-2, I-3 or I-4, wherein R² can be a C₃₋₈ cycloalkyl optionally substituted with one or more (e.g., 1, 2, or 3) substituents independently selected from F, CN, G¹, OH, COOH, C(O)-G¹, O-G¹, C(O)—O-G¹, NH₂, NH(G¹), N(G¹)(G¹), C(O)—NH₂, C(O)—NH(G¹), C(O)—N(G¹)(G¹), wherein G¹ at each occurrence is independently a C₁₋₄ alkyl optionally substituted with 1-3 substituents independently selected from F, CN, OH, and C₁₋₄ heteroalkyl, or a C₃₋₆ cycloalkyl optionally substituted with 1-3 substituents independently selected from F, CN, OH, and C₁₋₄ heteroalkyl. In some embodiments, in Formula I-1, I-2, I-3 or I-4, R² can be a C₃₋₆ cycloalkyl, such as cyclopropyl, cyclobutyl, cyclopentyl, cyclohexyl, which is optionally substituted with one or more (e.g., 1, 2, or 3) substituents independently selected from F, methyl, ethyl, hydroxyethyl (e.g., —CH₂CH₂OH or —CH(OH)CH₃), —C(O)CH₃, OH, —CH₂OH, fluorine substituted methyl (e.g., —CF₂H), and fluorine substituted ethyl (e.g., —CH₂CF₂H). In some embodiments, in Formula I-1, I-2, I-3 or I-4, R² can be a spiro, fused, or bridged C₆-s cycloalkyl, such as

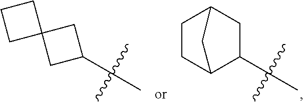 [SMILES: *C1CC2(CCC2)C1 ; *C1CC2CCC1C2]

which is optionally substituted with one or more (e.g., 1, 2, or 3) substituents independently selected from F, methyl, ethyl, hydroxyethyl (e.g., —CH₂CH₂OH or —CH(OH)CH₃), —C(O)CH₃, OH, —CH₂OH, fluorine substituted methyl (e.g., —CF₂H), and fluorine substituted ethyl (e.g., —CH₂CF₂H). For example, in any of the embodiments herein, unless specified or otherwise contrary from context, in Formula I-1, I-2, I-3 or I-4, R² can be selected from the following:

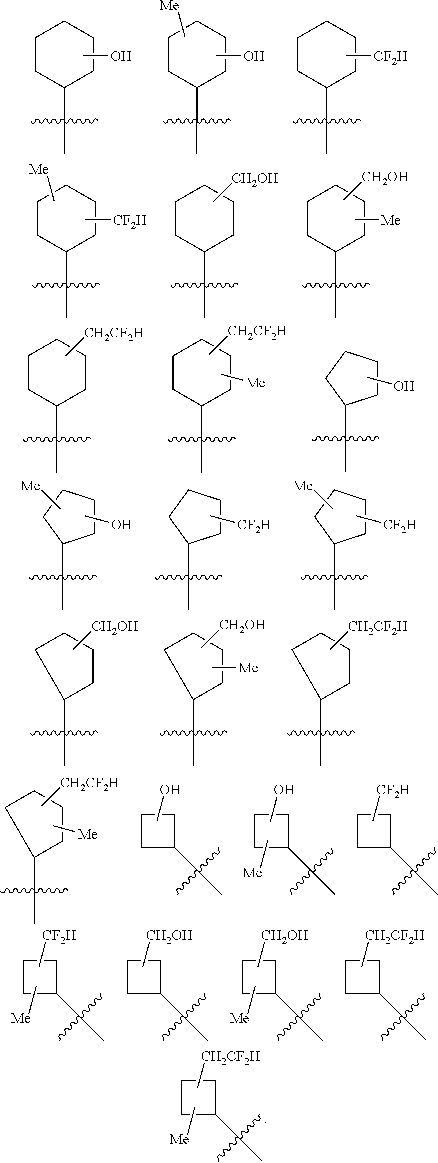

In any of the embodiments herein, unless specified or otherwise contrary from context, in Formula I-1, I-2, I-3 or I-4, R² can be selected from the following:

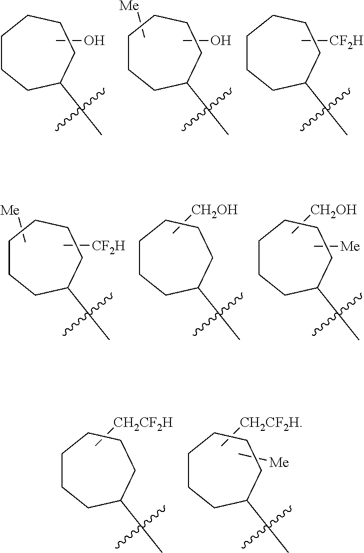

In any of the embodiments herein, unless specified or otherwise contrary from context, in Formula I-1, I-2, I-3 or I-4, R² can be selected from the following:

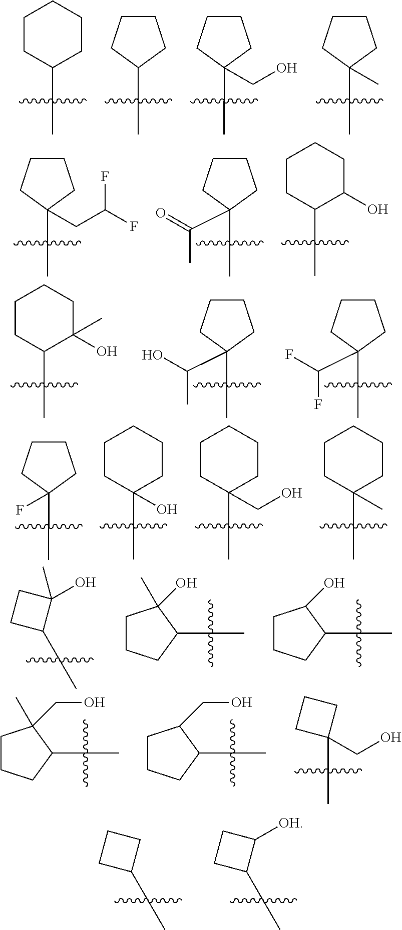

In any of the embodiments herein, unless specified or otherwise contrary from context, in Formula I-1 I-2 I-3 or I-4 R² can be selected from the following:

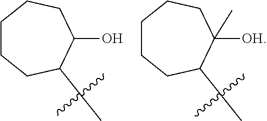

In any of the embodiments herein, unless specified or otherwise contrary from context, in Formula I-1, I-2, I-3 or I-4, R² can be selected from the following:

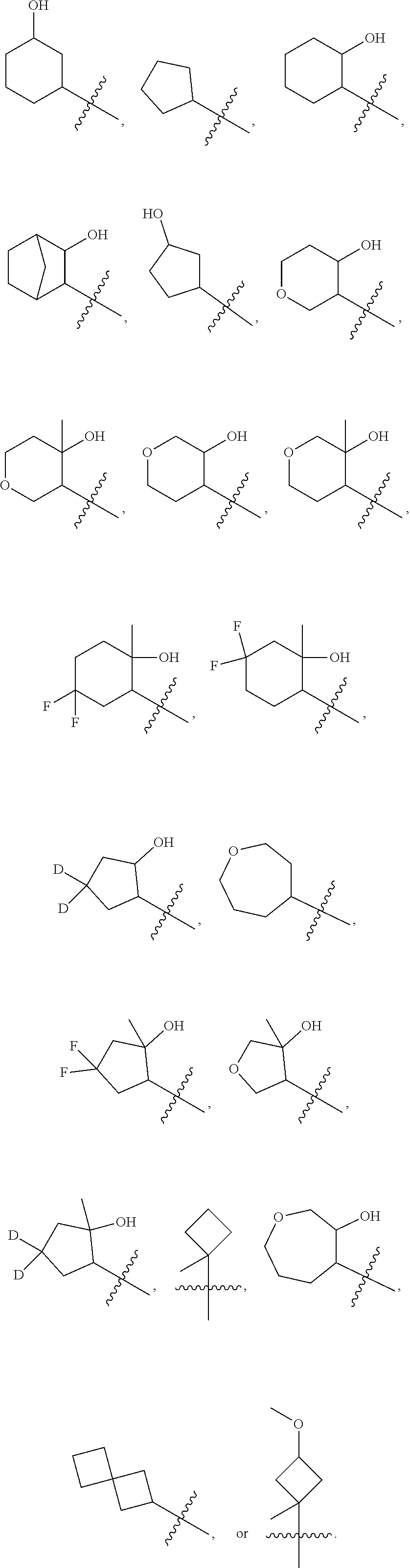

In some preferred embodiments, in Formula I-1, I-2, I-3 or I-4, R² can be selected from the following:

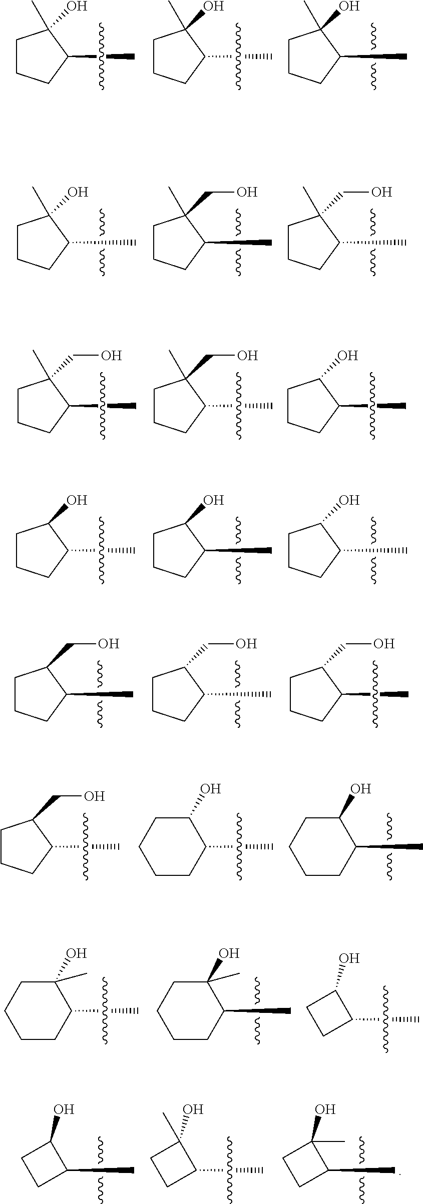

In some preferred embodiments, in Formula I-1, I-2, I-3 or I-4, R² can be selected from the following:

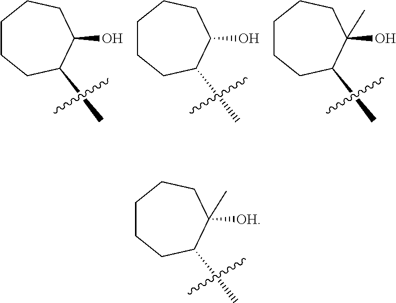

In some preferred embodiments, in Formula I-1, I-2, I-3 or I-4, R² can be selected from the following:

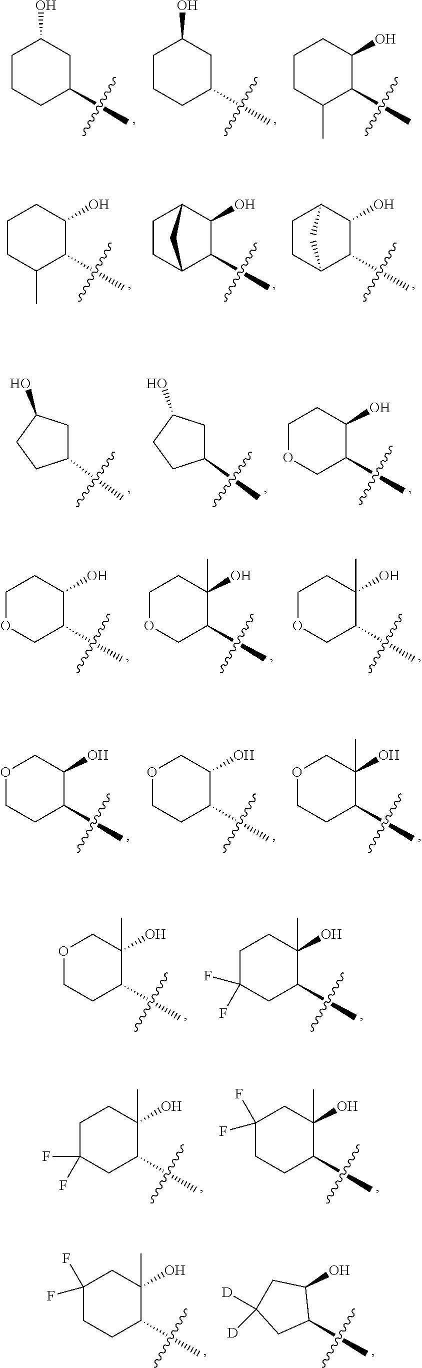

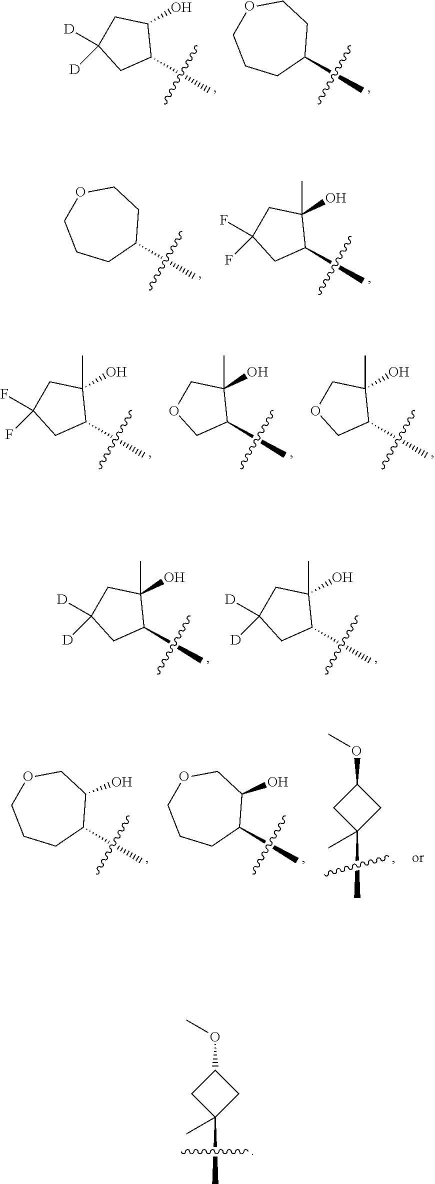

As shown in the Examples section, it was found that compounds of Formula I-1, I-2, I-3, or I-4 are potent CDK2 inhibitors, with some of the examples showing more than 10 fold selectivity over CDK1. Particularly, a representative compound, Example 9, showed more than 30 fold selectivity over CDK1. Additional compounds with more than 10 fold selectivity over CDK1 are also shown in the Examples herein.

In some embodiments, the compound of Formula I (e.g., Formula I-A, I-A-1, I-A-2, I-A-3, I-A-4, I-A-5, I-A-6, I-A-7, I-A-8, I-A-9, or I-A-10) can be characterized as having Formula I-2-1:

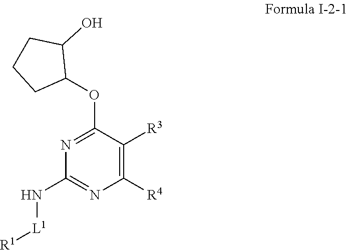

wherein L¹, R¹, R³, and R⁴ include any of those described herein in any combination. In some embodiments, the compound of Formula I-2-1 can be characterized as having Formula I-2-1-S1, I-2-1-S2, I-2-1-S3, or I-2-1-S4:

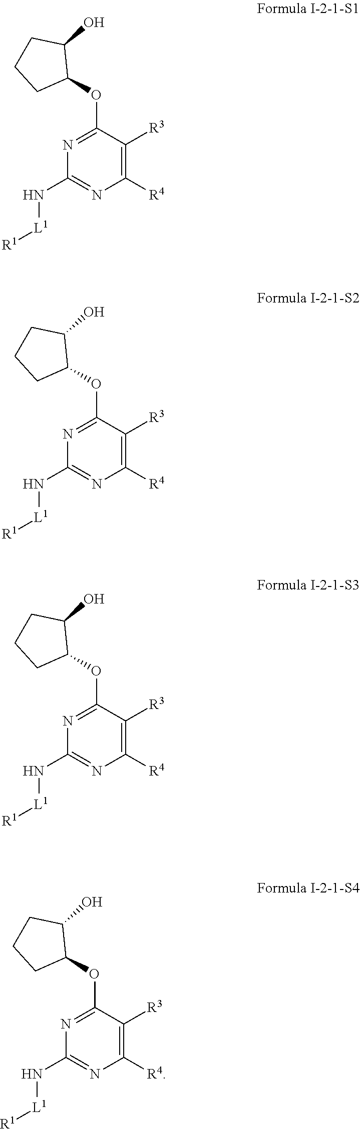

In some embodiments, the compound of any of Formula I-2-1-S1, I-2-1-S2, I-2-1-S3, and I-2-1-S4 can exist as a substantially pure stereoisomer, for example, substantially free (e.g., with less than 10%, less than 5%, less than 1%, by weight or by HPLC or SFC area, or non-detectable amount) of the other potential stereoisomers. For example, in some embodiments, the compound of Formula I-2-1-S1 can be a substantially pure stereoisomer, wherein out of the four potential stereoisomers, the combined amount of the corresponding stereoisomers of Formula I-2-1-S2, I-2-1-S3, and I-2-1-S4 that may be present is less than 10%, less than 5%, less than 1%, by weight or by HPLC or SFC area, or in a non-detectable amount. In some embodiments, the compound of Formula I-2-1 can also exist as a mixture of any two or more of the corresponding Formula I-2-1-S1, I-2-1-S2, I-2-1-S3, and I-2-1-S4 in any ratio.

In some preferred embodiments, the compound of Formula I (e.g., Formula I-A, I-A-1, I-A-2, I-A-3, I-A-4, I-A-5, I-A-6, I-A-7, I-A-8, I-A-9, or I-A-10) can be characterized as having Formula I-1, I-2, I-3 or I-4, wherein R² is a 4-8 membered heterocyclyl having 1-2 ring heteroatoms independently selected from N, O, and S, which is optionally substituted with one or more (e.g., 1, 2, or 3) substituents independently selected from oxo, F, CN, G¹, OH, COOH, C(O)-G¹, O-G¹, C(O)—O-G¹, NH₂, NH(G¹), N(G¹)(G¹), C(O)—NH₂, C(O)—NH(G¹), C(O)—N(G¹)(G¹), wherein G¹ at each occurrence is independently a C₁₋₄ alkyl optionally substituted with 1-3 substituents independently selected from F, CN, OH, and C₁₋₄ heteroalkyl, or a C₃₋₆ cycloalkyl optionally substituted with 1-3 substituents independently selected from F, CN, OH, and C₁₋₄ heteroalkyl. In some embodiments, in Formula I-1, I-2, I-3 or I-4, R² is a 4-6 membered monocyclic heterocyclyl having 1-2 ring heteroatoms independently selected from N, O, and S, such as oxetane, azetidine, tetrahydrofuran, tetrahydropyran, pyrrolidine, piperidine, etc., which is optionally substituted with one or more (e.g., 1, 2, or 3) substituents independently selected from oxo, F, methyl, ethyl, hydroxyethyl (e.g., —CH₂CH₂OH or —CH(OH)CH₃), —C(O)CH₃, OH, —CH₂OH, fluorine substituted methyl (e.g., —CF₂H), and fluorine substituted ethyl (e.g., —CH₂CF₂H). For example, in some embodiments, in Formula I-1, I-2, I-3 or I-4, R² can be selected from

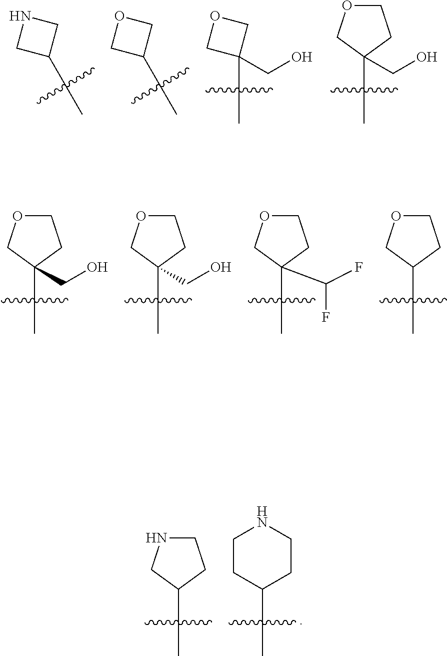

In some preferred embodiments, the compound of Formula I (e.g., Formula I-A, I-A-1, I-A-2, I-A-3, I-A-4, I-A-5, I-A-6, I-A-7, I-A-8, I-A-9, or I-A-10) can be characterized as having Formula I-1, I-2, I-3 or I-4, wherein R² can also be a 5- or 6-membered heteroaryl having 1-4 ring heteroatoms independently selected from N, O, and S, such as pyridyl (e.g., 2-, 3-, or 4-pyridyl), pyrazole, etc., which is optionally substituted with one or more (e.g., 1, 2, or 3) substituents independently selected from F, CN, G¹, OH, COOH, C(O)-G¹, O-G¹, C(O)—O-G¹, NH₂, NH(G¹), N(G¹)(G¹), C(O)—NH₂, C(O)—NH(G¹), C(O)—N(G¹)(G¹), G², O-G², NH(G²), N(G¹)(G²), C(O)—NH(G²), and C(O)—N(G¹)(G²), wherein G¹ at each occurrence is independently a C₁₋₄ alkyl optionally substituted with 1-3 substituents independently selected from F, CN, OH, and C₁₋₄ heteroalkyl, or a C₃₋₆ cycloalkyl optionally substituted with 1-3 substituents independently selected from F, CN, OH, and C₁₋₄ heteroalkyl; wherein G² at each occurrence is independently a 4-6 membered heterocyclyl having 1-2 ring heteroatoms independently selected from N, O, and S, phenyl or 5- or 6-membered heteroaryl, each of which is optionally substituted with 1-3 substituents independently selected from oxo (as applicable), F, CN, G¹, OH, O-G¹, NH₂, NH(G¹), and N(G¹)(G¹); and wherein two optional substituents of the heteroaryl group, together with the intervening atom(s), can optionally be joined to form a fused ring structure. For example, in some embodiments, in Formula I-1, I—2, I-3 or I-4, R² can also be selected from.

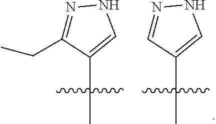

In some embodiments, in Formula I (e.g., Formula I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B, I-A-8B, I-A-9B, I-A-10B, or I-B), L² and L³ are both a bond, in which case R² is directly attached to the pyridine or pyrimidine ring of Formula I. For example, in some embodiments, the compound of Formula I (e.g., Formula I-A, I-A-1, I-A-2, I-A-3, I-A-4, I-A-5, I-A-6, I-A-7, I-A-8, I-A-9, or I-A-10) can be characterized as having Formula I-5:

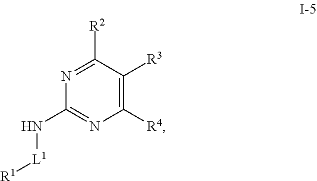

wherein L¹, R¹, R², R³, and R⁴ include any of those described herein in any combination.

In some embodiments, the compound of Formula I (e.g., Formula I-A, I-A-1, I-A-2, I-A-3, I-A-4, I-A-5, I-A-6, I-A-7, I-A-8, I-A-9, or I-A-10) can be characterized as having Formula I-5, wherein R² can be a 4-10 membered heterocyclyl having 1-4 ring heteroatoms independently selected from N, O, and S, which is optionally substituted with one or more (e.g., 1, 2, or 3) substituents independently selected from oxo, F, CN, G¹, OH, COOH, C(O)-G¹, O-G¹, C(O)—O-G¹, NH₂, NH(G¹), N(G¹)(G¹), C(O)—NH₂, C(O)—NH(G¹), C(O)—N(G¹)(G¹), G², O-G², NH(G²), N(G¹)(G²), C(O)—NH(G²), and C(O)—N(G¹)(G²), wherein G¹ at each occurrence is independently a C₁₋₄ alkyl optionally substituted with 1-3 substituents independently selected from F, CN, OH, and C₁₋₄heteroalkyl, or a C₃₋₆ cycloalkyl optionally substituted with 1-3 substituents independently selected from F, CN, OH, and C₁₋₄ heteroalkyl; wherein G² at each occurrence is independently a 4-6 membered heterocyclyl having 1-2 ring heteroatoms independently selected from N, O, and S, phenyl or 5- or 6-membered heteroaryl, each of which is optionally substituted with 1-3 substituents independently selected from oxo (as applicable), F, CN, G¹, OH, O-G¹, NH₂, NH(G¹), and N(G¹)(G¹); and wherein two optional substituents of the 4-10 membered heterocyclyl, together with the intervening atom(s), can optionally be joined to form a fused, bridged, or spiro ring structure.

In some preferred embodiments, the compound of Formula I (e.g., Formula I-A, I-A-1, I-A-2, I-A-3, I-A-4, I-A-5, I-A-6, I-A-7, I-A-8, I-A-9, or I-A-10) can be characterized as having Formula I-5, wherein R² is a 4-8 membered monocyclic, saturated or partially unsaturated, heterocyclyl having 1-2 ring heteroatoms independently selected from N, O, and S, such as pyrrolidine, piperidine, azepane, etc., which is optionally substituted with one or more (e.g., 1, 2, or 3) substituents independently selected from oxo, F, CN, G¹, OH, COOH, C(O)-G¹, O-G¹, C(O)—O-G¹, NH₂, NH(G¹), N(G¹)(G¹), C(O)—NH₂, C(O)—NH(G¹), C(O)—N(G¹)(G¹), G², O-G², NH(G²), N(G¹)(G²), C(O)—NH(G²), and C(O)—N(G¹)(G²), wherein G¹ at each occurrence is independently a C₁₋₄ alkyl optionally substituted with 1-3 substituents independently selected from F, CN, OH, and C₁₋₄heteroalkyl, or a C₃₋₆ cycloalkyl optionally substituted with 1-3 substituents independently selected from F, CN, OH, and C₁₋₄ heteroalkyl; wherein G² at each occurrence is independently a 4-6 membered heterocyclyl having 1-2 ring heteroatoms independently selected from N, O, and S, phenyl or 5- or 6-membered heteroaryl, each of which is optionally substituted with 1-3 substituents independently selected from oxo (as applicable), F, CN, G¹, OH, O-G¹, NH₂, NH(G¹), and N(G¹)(G¹); and wherein two optional substituents of the 4-8 membered heterocyclyl, together with the intervening atom(s), can optionally be joined to form a fused, bridged, or spiro ring structure.

In some preferred embodiments, the compound of Formula I (e.g., Formula I-A, I-A-1, I-A-2, I-A-3, I-A-4, I-A-5, I-A-6, I-A-7, I-A-8, I-A-9, or I-A-10) can be characterized as having Formula I-5, wherein R² can be selected from

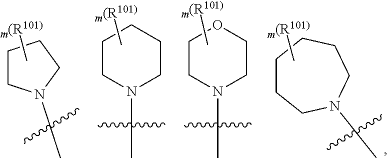

wherein:
m is 0, 1, 2, 3, or 4;
R¹⁰¹ at each occurrence is independently oxo, F, CN, G¹, G², OH, O-G¹, and O-G², wherein G¹ at each occurrence is independently a C₁₋₄ alkyl optionally substituted with 1-3 substituents independently selected from F, CN, OH, and C₁₋₄ heteroalkyl, or a C₃₋₆ cycloalkyl optionally substituted with 1-3 substituents independently selected from F, CN, OH, and C₁₋₄ heteroalkyl; wherein G² at each occurrence is independently 4-6 membered heterocyclyl having 1-2 ring heteroatoms independently selected from N, O, and S, phenyl or 5- or 6-membered heteroaryl having 1-4 ring heteroatoms independently selected from N, O, and S, each of which is optionally substituted with 1-3 substituents independently selected from F, CN, G¹, OH, and O-G¹; wherein two R¹⁰¹, together with the intervening atom(s), can optionally be joined to form a fused, bridged, or spiro ring structure. In some embodiments, m can be 0, 1, 2, or 3. For example, in some embodiments, m is 0, i.e., the heterocyclyl is not substituted. In some embodiments, m is 1. In some embodiments, m is 2. In some embodiments, R¹⁰¹ at each occurrence is independently F, OH, CN, C₁₋₄ alkyl (e.g., methyl, ethyl, propyl, etc.) phenyl, cyclopropyl, hydroxymethyl (—CH₂OH), methoxy, fluorine substituted methoxy, fluorine substituted C₁₋₄ alkyl, such as fluorine substituted methyl such as CF₂H, or fluorine substituted ethyl (e.g., CH₂CF₂H).

In any of the embodiments herein, unless specified or otherwise contrary from context, in Formula I-5, R² can be selected from:

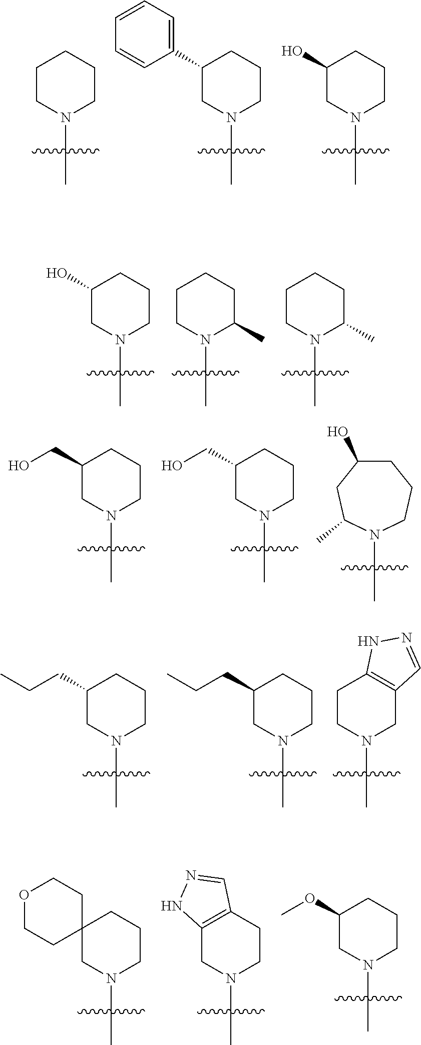

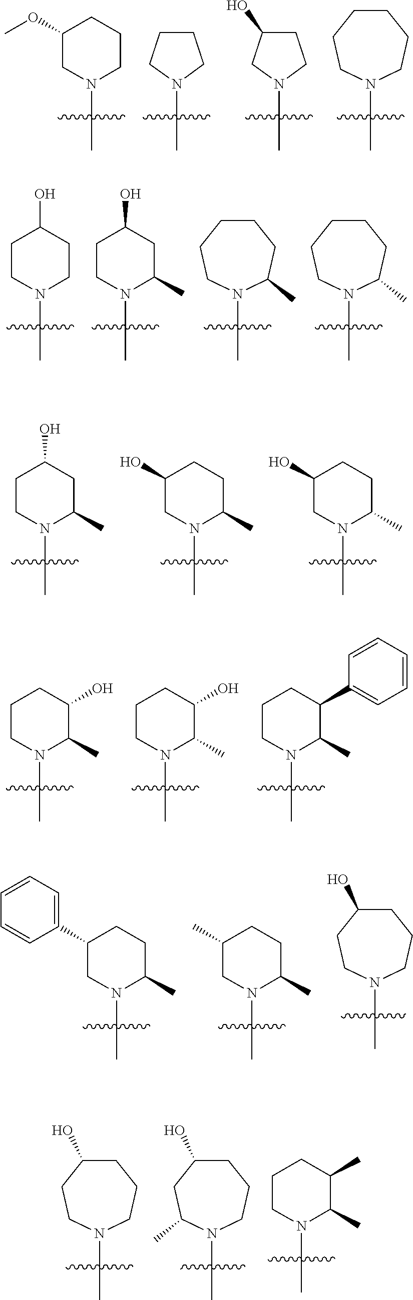

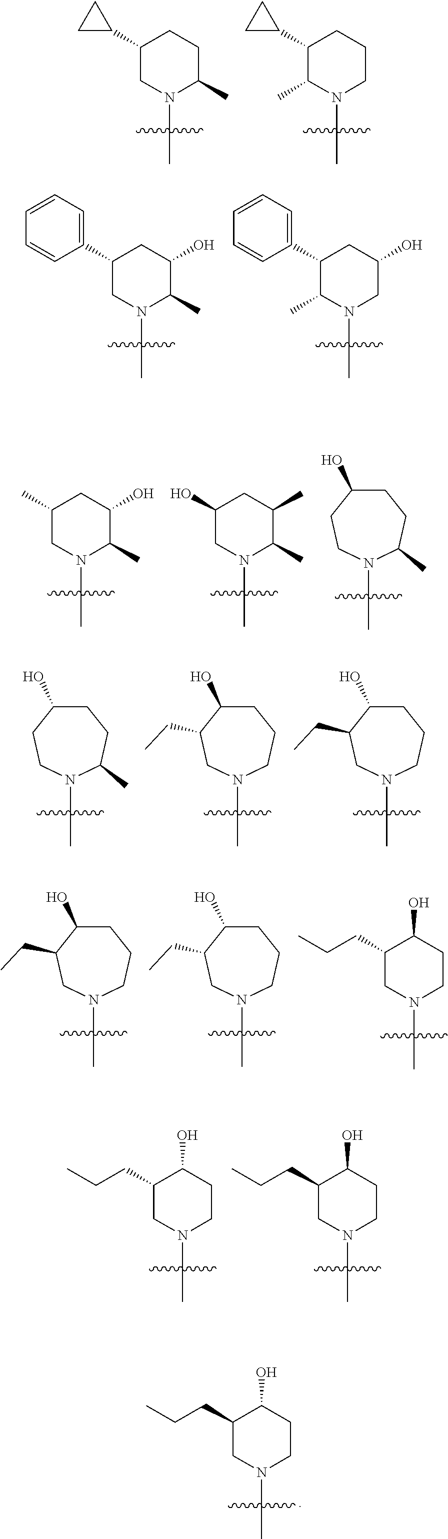

In some embodiments, the compound of Formula I (e.g., Formula I-A, I-A-1, I-A-2, I-A-3, I-A-4, I-A-5, I-A-6, I-A-7, I-A-8, I-A-9, or I-A-10) can be characterized as having Formula I-5, wherein R² can be a phenyl optionally substituted with one or more (e.g., 1, 2, or 3) substituents independently selected from F, CN, G¹, OH, COOH, C(O)-G¹, O-G¹, C(O)—O-G¹, NH₂, NH(G¹), N(G¹)(G¹), C(O)—NH₂, C(O)—NH(G¹), C(O)—N(G¹)(G¹), G², O-G², NH(G²), N(G¹)(G²), C(O)—NH(G²), and C(O)—N(G¹)(G²), wherein G¹ at each occurrence is independently a C₁₋₄ alkyl optionally substituted with 1-3 substituents independently selected from F, CN, OH, and C₁₋₄ heteroalkyl, or a C₃₋₆ cycloalkyl optionally substituted with 1-3 substituents independently selected from F, CN, OH, and C₁₋₄ heteroalkyl; wherein G² at each occurrence is independently a 4-6 membered heterocyclyl having 1-2 ring heteroatoms independently selected from N, O, and S, phenyl or 5- or 6-membered heteroaryl, each of which is optionally substituted with 1-3 substituents independently selected from oxo (as applicable), F, CN, G¹, OH, O-G¹, NH₂, NH(G¹), and N(G¹)(G¹); wherein two optional substituents of the phenyl group, together with the intervening atom(s), can optionally be joined to form a fused ring structure.

For example, in some preferred embodiments, in Formula I-5, R² can be

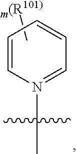

wherein:
m is 0, 1, 2, or 3;
R¹⁰¹ at each occurrence is independently F, CN, G¹, G², OH, O-G¹, O-G², NH₂, NH(G¹), NH(G²), N(G¹)(G¹), and N(G¹)(G²), wherein G¹ at each occurrence is independently a C₁₋₄ alkyl optionally substituted with 1-3 substituents independently selected from F, OH, and C₁₋₄ heteroalkyl or a C₃₋₆ cycloalkyl optionally substituted with 1-3 substituents independently selected from F, OH, and C₁₋₄heteroalkyl; wherein G² at each occurrence is independently 4-6 membered heterocyclyl having 1-2 ring heteroatoms independently selected from N, O, and S, phenyl or 5- or 6-membered heteroaryl having 1-4 ring heteroatoms independently selected from N, O, and S, each of which is optionally substituted with 1-3 substituents independently selected from F, CN, G¹, OH, and O-G¹; wherein two R¹⁰¹, together with the intervening atom(s), can optionally be joined to form a fused ring structure. In some embodiments, m can be 0, 1, 2, or 3. For example, in some embodiments, m is 0, i.e., the phenyl is not substituted. In some embodiments, m is 1. In some embodiments, m is 2. In some embodiments, m is 3. In some embodiments, R¹⁰¹ at each occurrence is independently F, OH, CN, C₁₋₄ alkyl (e.g., methyl, ethyl, propyl, etc.), cyclopropyl, cyclobutyl, oxetanyl, C₁₋₄ alkoxy (e.g., methoxy), fluorine substituted C₁₋₄ alkoxy such as fluorine substituted methoxy, fluorine substituted C₁₋₄ alkyl, such as fluorine substituted methyl such as CF₂H, or fluorine substituted ethyl (e.g., CH₂CF₂H). In some embodiments, R¹⁰¹ at each occurrence is independently F, C₁₋₄ alkyl (e.g., methyl, ethyl, n-propyl, etc.), OH, cyclopropyl, cyclobutyl, oxetanyl, or CN.

In any of the embodiments herein, unless specified or otherwise contrary from context, in Formula I-5, R² can be selected from:

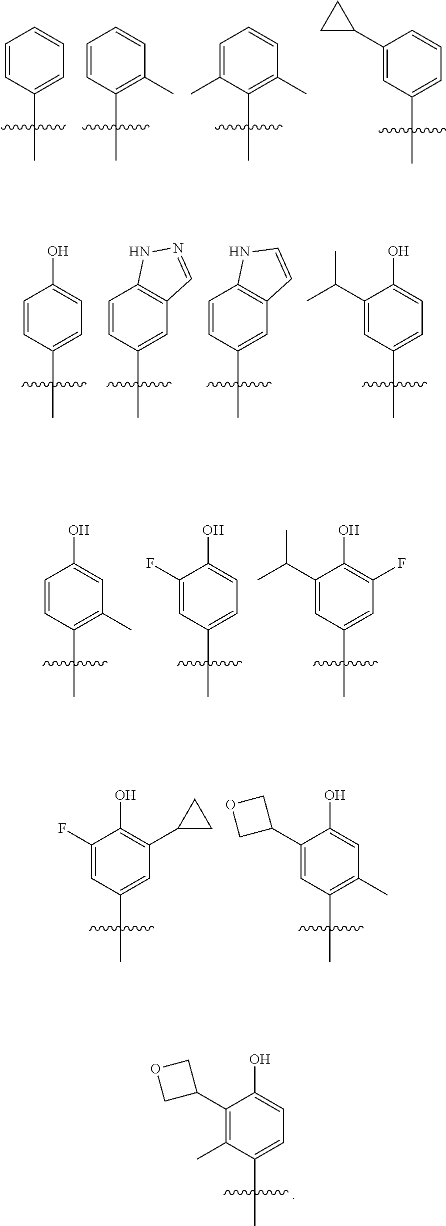

In some preferred embodiments, the compound of Formula I (e.g., Formula I-A, I-A-1, I-A-2, I-A-3, I-A-4, I-A-5, I-A-6, I-A-7, I-A-8, I-A-9, or I-A-10) can be characterized as having Formula I-5, wherein R² can also be a 5-10 membered heteroaryl having 1-4 ring heteroatoms independently selected from N, O, and S, which is optionally substituted with one or more (e.g., 1, 2, or 3) substituents independently selected from F, CN, G¹, OH, COOH, C(O)-G¹, O-G¹, C(O)—O-G¹, NH₂, NH(G¹), N(G¹)(G¹), C(O)—NH₂, C(O)—NH(G¹), C(O)—N(G¹)(G¹), G², O-G², NH(G²), N(G¹)(G²), C(O)—NH(G²), and C(O)—N(G¹)(G²), wherein G¹ at each occurrence is independently a C₁₋₄ alkyl optionally substituted with 1-3 substituents independently selected from F, CN, OH, and C₁₋₄heteroalkyl, or a C₃₋₆ cycloalkyl optionally substituted with 1-3 substituents independently selected from F, CN, OH, and C₁₋₄ heteroalkyl; wherein G² at each occurrence is independently a 4-6 membered heterocyclyl having 1-2 ring heteroatoms independently selected from N, O, and S, phenyl or 5- or 6-membered heteroaryl, each of which is optionally substituted with 1-3 substituents independently selected from oxo (as applicable), F, CN, G¹, OH, O-G¹, NH₂, NH(G¹), and N(G¹)(G¹); and wherein two optional substituents of the heteroaryl group, together with the intervening atom(s), can optionally be joined to form a fused ring structure.

In some preferred embodiments, the compound of Formula I (e.g., Formula I-A, I-A-1, I-A-2, I-A-3, I-A-4, I-A-5, I-A-6, I-A-7, I-A-8, I-A-9, or I-A-10) can be characterized as having Formula I-5, wherein R² can be a 5- or 6-membered heteroaryl having 1-4 ring heteroatoms independently selected from N, O, and S, such as pyridyl (e.g., 2-, 3-, or 4-pyridyl), pyrazole, etc., which is optionally substituted with one or more (e.g., 1, 2, or 3) substituents independently selected from F, CN, G¹, OH, COOH, C(O)-G¹, O-G¹, C(O)—O-G¹, NH₂, NH(G¹), N(G¹)(G¹), C(O)—NH₂, C(O)—NH(G¹), C(O)—N(G¹)(G¹), G², O-G², NH(G²), N(G¹)(G²), C(O)—NH(G²), and C(O)—N(G¹)(G²), wherein G¹ at each occurrence is independently a C₁₋₄ alkyl optionally substituted with 1-3 substituents independently selected from F, CN, OH, and C₁₋₄ heteroalkyl, or a C₃₋₆ cycloalkyl optionally substituted with 1-3 substituents independently selected from F, CN, OH, and C₁₋₄ heteroalkyl; wherein G² at each occurrence is independently a 4-6 membered heterocyclyl having 1-2 ring heteroatoms independently selected from N, O, and S, phenyl or 5- or 6-membered heteroaryl, each of which is optionally substituted with 1-3 substituents independently selected from oxo (as applicable), F, CN, G¹, OH, O-G¹, NH₂, NH(G¹), and N(G¹)(G¹); and wherein two optional substituents of the heteroaryl group, together with the intervening atom(s), can optionally be joined to form a fused ring structure. For example, in some embodiments, in Formula I-5, R² can be selected from

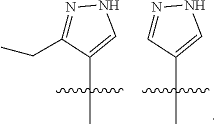

In some preferred embodiments, the compound of Formula I (e.g., Formula I-A, I-A-1, I-A-2, I-A-3, I-A-4, I-A-5, I-A-6, I-A-7, I-A-8, I-A-9, or I-A-10) can be characterized as having Formula I-5, wherein R² can be a 8-10-membered bicyclic heteroaryl having 1-4 ring heteroatoms independently selected from N, O, and S, such as indolyl, indazolyl, etc., which is optionally substituted with one or more (e.g., 1, 2, or 3) substituents independently selected from F, CN, G¹, OH, COOH, C(O)-G¹, O-G¹, C(O)—O-G¹, NH₂, NH(G¹), N(G¹)(G¹), C(O)—NH₂, C(O)—NH(G¹), C(O)—N(G¹)(G¹), G², O-G², NH(G²), N(G¹)(G²), C(O)—NH(G²), and C(O)—N(G¹)(G²), wherein G¹ at each occurrence is independently a C₁₋₄ alkyl optionally substituted with 1-3 substituents independently selected from F, CN, OH, and C₁₋₄ heteroalkyl, or a C₃₋₆ cycloalkyl optionally substituted with 1-3 substituents independently selected from F, CN, OH, and C₁₋₄heteroalkyl; wherein G² at each occurrence is independently a 4-6 membered heterocyclyl having 1-2 ring heteroatoms independently selected from N, O, and S, phenyl or 5- or 6-membered heteroaryl, each of which is optionally substituted with 1-3 substituents independently selected from oxo (as applicable), F, CN, G¹, OH, O-G¹, NH₂, NH(G¹), and N(G¹)(G¹); and wherein two optional substituents of the heteroaryl group, together with the intervening atom(s), can optionally be joined to form a fused ring structure.

In some preferred embodiments, the compound of Formula I-5 can be characterized as having Formula I-5-1 or I-5-2:

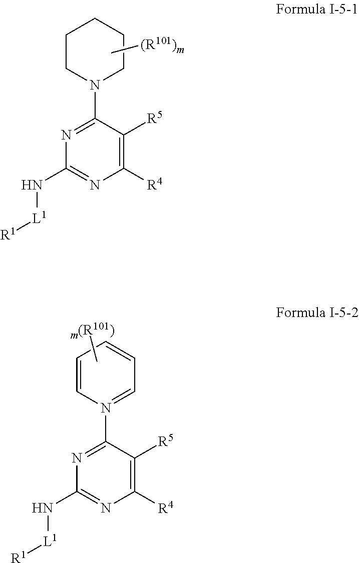

wherein L¹, R¹, R³, R⁴, m, and R¹⁰¹ include any of those described herein in any combination.

Various groups are suitable for R³ in Formula I. For example, in some embodiments, R³ is hydrogen. In some embodiments, R³ is halogen (e.g., F). In some embodiments, R³ is CN. In some embodiments, R³ is C(O)NR¹¹R¹², wherein R¹¹ and R¹² are defined herein, for example, both R¹¹ and R¹² can be hydrogen. In some embodiments, R³ is an optionally substituted C₃-s carbocyclyl. In some embodiments, R³ is an optionally substituted 4-10 membered heterocyclyl having 1 or 2 ring heteroatoms independently selected from N, O, and S. In some embodiments, R³ is an optionally substituted 5-10 membered heteroaryl having 1-4 ring heteroatoms independently selected from N, O, and S.

In any of the embodiments herein, unless specified or otherwise contrary from context, R³ in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, I-A-1, I-A-2, I-A-3, I-A-4, or I-B) can be hydrogen, F, Cl, Br, C₁₋₄ alkyl optionally substituted with F, or CN. For example, in some embodiments, the compound of Formula I can be characterized as having a formula according to Formula I-A-5A, I-A-6A, I-A-7A, I-A-8A, I-A-9A, I-A-10A, I-A-5B, I-A-6B, I-A-7B, I-A-8B, I-A-9B, or I-A-10B:

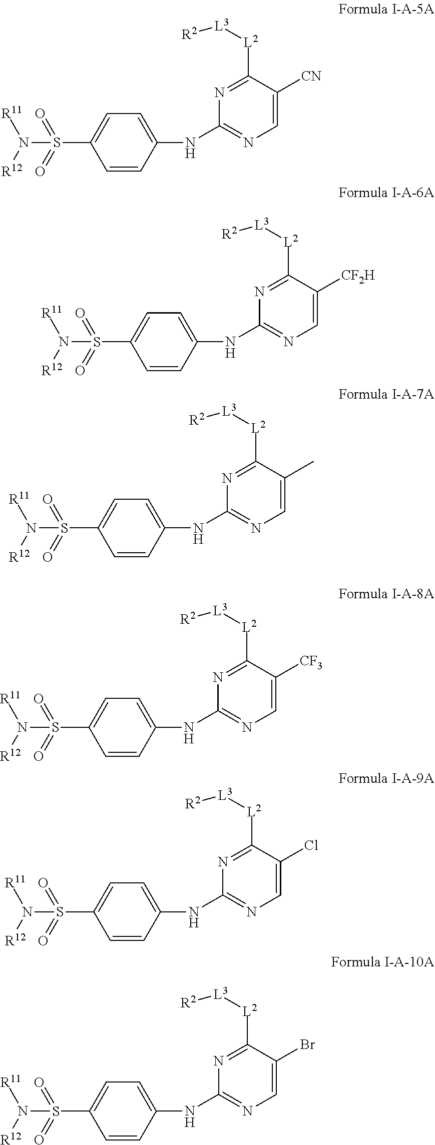

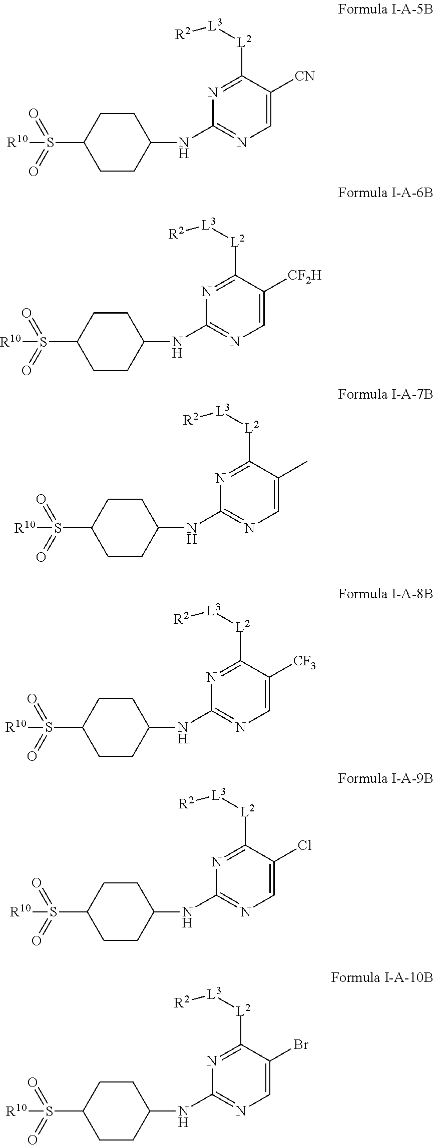

wherein L², L³, R², R¹⁰, R¹¹, and R¹² include any of those described herein in any combination. In some embodiments according to formula I-A-5A, I-A-6A, I-A-7A, I-A-8A, I-A-9A, or I-A-10A, R¹¹ and R¹² are independently hydrogen, C₁₋₄ alkyl optionally substituted with F and/or deuterium, or C₃₋₆ cycloalkyl optionally substituted with F and/or deuterium. In some embodiments according to formula I-A-5A, I-A-6A, I-A-7A, I-A-8A, I-A-9A, or I-A-10A, one of R¹¹ and R¹² is hydrogen, and the other of R¹¹ and R¹² is hydrogen, C₁₋₄ alkyl optionally substituted with F and/or deuterium, or C₃₋₆ cycloalkyl optionally substituted with F and/or deuterium. In some preferred embodiments according to formula I-A-5A, I-A-6A, I-A-7A, I-A-8A, I-A-9A, or I-A-10A, one of R¹¹ and R¹² is hydrogen, and the other of R¹¹ and R¹² is hydrogen, methyl, CD₃, ethyl, isopropyl, cyclopropyl, cyclobutyl,

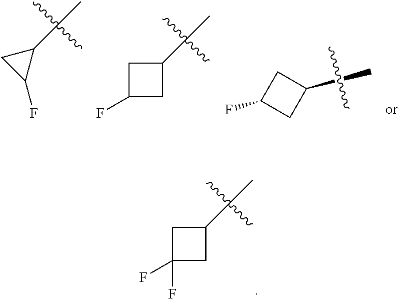

In some preferred embodiments according to formula I-A-5B, I-A-6B, I-A-7B, I-A-8B, I-A-9B, or I-A-10B, R¹⁰ is C₁₋₄ alkyl optionally substituted with 1-3 F, such as CH₃, CH₂F, CF₃, etc. In some preferred embodiments according to formula I-A-5B, I-A-6B, I-A-7B, I-A-8B, I-A-9B, or I-A-10B, R¹⁰ is a 5 or 6 membered heteroaryl having 1-3 ring heteroatoms independently selected from N, O, and S, such as pyrrazole, imidazole, triazole, etc., which can be optionally substituted, for example, with a C₁₋₄ alkyl (e.g., methyl), for example,

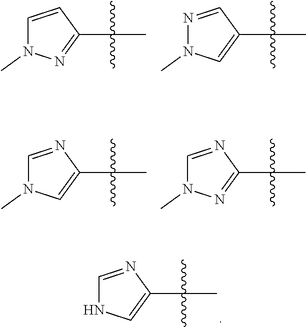

In some embodiments, R³ in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, I-A-1, I-A-2, I-A-3, I-A-4, or I-B) can be an optionally substituted C₁₋₄ alkyl. In some embodiments, R³ can be C₁₋₄ alkyl optionally substituted with one or more, such as 1-3 substituents independently selected from deuterium, F, CN, or OR^{C}, wherein R^{C} at each occurrence is independently hydrogen, C₁₋₄ alkyl optionally substituted with 1-3 substituents independently selected from deuterium, F, CN, OH, and C₁₋₄ heteroalkyl, or a C₃₋₆ cycloalkyl optionally substituted with 1-3 substituents independently selected from deuterium, F, CN, OH, and C₁₋₄ heteroalkyl. For example, in some embodiments, R³ can be methyl, CD₃, CH₂-OMe, CH₂—OCD₃, ethyl, CHF₂, CF₂CH₃, CH₂CH₂F, CH₂CF₂H, or CF₃.

In some embodiments, R³ in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, I-A-1, I-A-2, I-A-3, I-A-4, or I-B) can be an optionally substituted C₂₋₄ alkenyl, such as

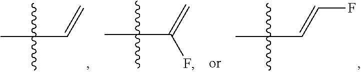

In some embodiments, R³ in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, I-A-1, I-A-2, I-A-3, I-A-4, or I-B) can be an optionally substituted C₂₋₄ alkynyl, such as.

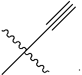 [SMILES: *C#C]

In some embodiments, R³ in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, I-A-1, I-A-2, I-A-3, I-A-4, or I-B) can be OR^{A}. For example, in some embodiments, R³ is OR^{A}, and R^{A} is hydrogen, C₁₋₄ alkyl optionally substituted with 1-3 substituents independently selected from deuterium, F, CN, OH, and C₁₋₄ heteroalkyl, or a C₃₋₆ cycloalkyl optionally substituted with 1-3 substituents independently selected from deuterium, F, CN, OH, and C₁₋₄ heteroalkyl.

In some embodiments, R³ in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, I-A-1, I-A-2, I-A-3, I-A-4, or I-B) can be C(O)RB. For example, in some embodiments, R³ is C(O)RB and RB is hydrogen, C₁₋₄ alkyl optionally substituted with 1-3 substituents independently selected from deuterium, F, CN, OH, and C₁₋₄ heteroalkyl, or a C₃₋₆ cycloalkyl optionally substituted with 1-3 substituents independently selected from deuterium, F, CN, OH, and C₁₋₄ heteroalkyl.

In some embodiments, R³ in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, I-A-1, I-A-2, I-A-3, I-A-4, or I-B) can also be a C₃₋₆ cycloalkyl (e.g., cyclopropyl, cyclobutyl, cyclopentyl, etc.), 4-6 membered heterocyclyl having 1-2 ring heteroatoms independently selected from N, O, and S, such as oxetanyl, tetrahydrofuranyl, or 5-6 membered heteroaryl having 1-4 ring heteroatoms independently selected from N, O, and S, such as thiazolyl, each of which is optionally substituted with 1-3 substituents independently selected from oxo (as applicable), deuterium, F, CN, G¹, OH, O-G¹, NH₂, NH(G¹), N(G¹)(G¹), C(O)—NH₂, C(O)—NH(G¹), and C(O)—N(G¹)(G¹), wherein G¹ at each occurrence is independently a C₁₋₄ alkyl optionally substituted with 1-3 substituents independently selected from deuterium, F, CN, OH, and C₁₋₄ heteroalkyl, or a C₃₋₆ cycloalkyl optionally substituted with 1-3 substituents independently selected from deuterium, F, CN, OH, and C₁₋₄ heteroalkyl.

In any of the embodiments herein, unless specified or otherwise contrary from context, R³ in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, I-A-1, I-A-2, I-A-3, I-A-4, or I-B) can be selected from:

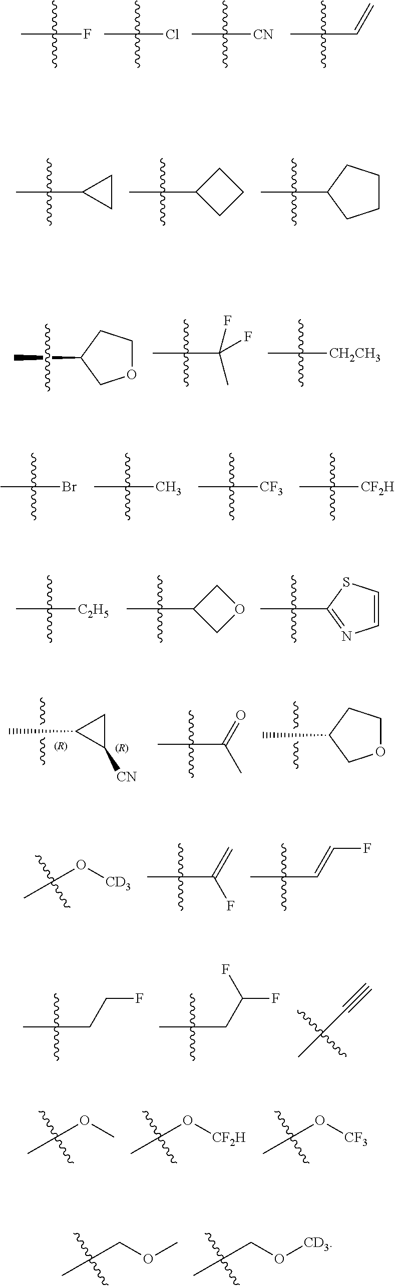

R⁴ in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, I-A-1, I-A-2, I-A-3, I-A-4, or I-B) is typically hydrogen. In some embodiments, R⁴ in Formula I can also be a halogen (e.g., F), optionally substituted C₁₋₆ alkyl, or NR¹¹R¹². For example, in some embodiments, R⁴ in Formula I is NH₂.

In some embodiments, in Formula I (e.g., Formula I-A, I-A-1, I-A-2, I-A-3, I-A-4, or I-B), when applicable, L² and R³, together with the intervening atoms, can also be joined to form an optionally substituted 4-8 membered ring structure, such as 4-8 membered heterocyclic structure or 5 or 6 membered heteroaryl structure.

In some embodiments, in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, I-A-1, I-A-2, I-A-3, I-A-4, or I-B), R³ and R⁴, together with the intervening atoms, can also be joined to form an optionally substituted 4-8 membered ring structure, such as 4-8 membered heterocyclic structure or 5 or 6 membered heteroaryl structure. For example, in any of the embodiments herein, unless specified or otherwise contrary from context, in Formula I (e.g., Formula I-1, I-2, I-3, I-4, I-5, I-2-1, I-2-1-S1, I-2-1-S2, I-2-1-S3, I-2-1-S4, I-5-1, I-5-2, I-A, I-A-1, I-A-2, I-A-3, I-A-4, or I-B), R³ and R⁴, together with the intervening atoms, can be joined to form one of the following:

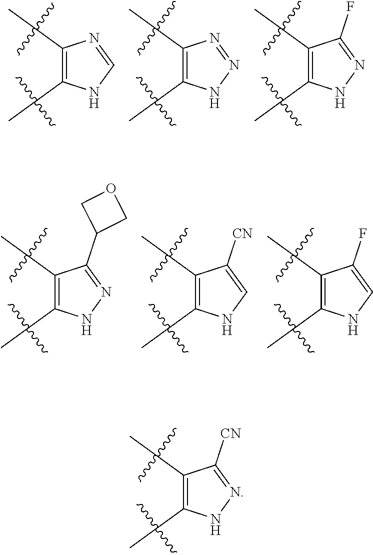

In some embodiments, the present disclosure provides a compound of Formula II, or a pharmaceutically acceptable salt thereof:

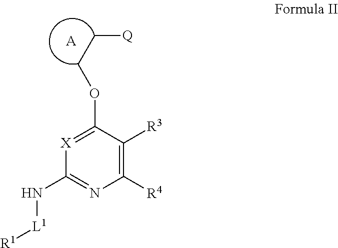

wherein:
L¹ is an optionally substituted arylene (e.g., phenylene), optionally substituted heteroarylene (e.g., 5- or 6-membered heteroarylene), optionally substituted heterocyclylene (e.g., 4-8-membered heterocyclylene), or optionally substituted carbocyclylene (e.g., C₃₋₈ carbocyclylene);
R¹ is SO₂R¹⁰, SO₂NR¹¹R¹², S(O)(NH)R¹⁰, or C(O)NR¹¹R¹²; or R¹ is hydrogen or NR¹¹R¹²;
X is N or CR¹³;
Ring A is an optionally substituted carbocyclic ring or optionally substituted heterocyclic ring having one or more (e.g., 1 or 2) ring heteroatoms independently selected from O, N, and S;
Q is hydrogen, OR^{A}, optionally substituted C₁₋₄ alkyl, halogen, CN, or COR^{B}; R³ is hydrogen, halogen (e.g., F), CN, C(O)NR¹¹R¹², optionally substituted C₁₋₆ alkyl, optionally substituted C₂₋₄ alkenyl, optionally substituted C₂₋₄ alkynyl, optionally substituted C₁₋₄ heteroalkyl, OR^{A}, COR^{B}, COOR^{A} NR¹¹R¹², optionally substituted C₃-s carbocyclyl, optionally substituted 4-10 membered heterocyclyl, or optionally substituted 5-10 membered heteroaryl;
R⁴ is hydrogen, halogen (e.g., F), optionally substituted C₁₋₆ alkyl, or NR¹¹R¹² or R³ and R⁴, together with the intervening atoms, form an optionally substituted 4-8 membered ring structure;
wherein:
R¹⁰ is an optionally substituted C₁₋₆ alkyl, optionally substituted C₃₋₈ carbocyclyl, optionally substituted phenyl, optionally substituted heteroaryl (e.g., 5- or 6-membered heteroaryl), or optionally substituted 4-10 membered heterocyclyl;
each of R¹¹ and R¹², at each occurrence, is independently hydrogen, an optionally substituted C₁₋₆ alkyl, optionally substituted C₃-s carbocyclyl, optionally substituted phenyl, optionally substituted heteroaryl (e.g., 5- or 6-membered heteroaryl), optionally substituted 4-10 membered heterocyclyl; or a nitrogen protecting group; or R¹¹ and R¹² can be joined to form an optionally substituted 4-10 membered heterocyclyl or 5- or 6-membered heteroaryl;
R^{A} at each occurrence is independently hydrogen, an optionally substituted C₁₋₆ alkyl, optionally substituted C₃₋₈ carbocyclyl, optionally substituted phenyl, optionally substituted heteroaryl (e.g., 5- or 6-membered heteroaryl), optionally substituted 4-10 membered heterocyclyl; or an oxygen protecting group;
R^{B} at each occurrence is independently hydrogen, an optionally substituted C₁₋₆ alkyl, optionally substituted C₃₋₈ carbocyclyl, optionally substituted phenyl, optionally substituted 4-10 membered heterocyclyl, or optionally substituted heteroaryl (e.g., 5- or 6-membered heteroaryl); and
R¹³ is hydrogen, F, CN, —OH, an optionally substituted C₁₋₄ alkyl, optionally substituted C₁₋₄ heteroalkyl, optionally substituted C₃-s carbocyclyl, or optionally substituted 4-10 membered heterocyclyl.
To be clear, Ring A as drawn in Formula II (including any of the applicable subformulae) should be understood as containing at least two ring carbon atoms connecting to the O atom and Q group as drawn in Formula II, respectively.

In some embodiments, the compound of Formula II (including any of the applicable sub-formulae as described herein) can exist in various stereoisomeric forms, e.g., enantiomers and/or diastereomers. In some embodiments, the compound of Formula II can exist in the form of an individual enantiomer and/or diastereomer, as applicable, or a mixture of stereoisomers, including racemic mixtures and mixtures enriched in one or more stereoisomers. In some embodiments, when applicable, the compound of Formula II (including any of the applicable sub-formulae as described herein) can exist as an isolated individual enantiomer substantially free (e.g., with less than 20%, less than 10%, less than 5%, less than 1%, by weight, by HPLC or SFC area, or both, or with a non-detectable amount) of the other enantiomer. In some embodiments, when applicable, the compound of Formula II (including any of the applicable sub-formulae as described herein) can also exist as a mixture of stereoisomers in any ratio, such as a racemic mixture.

It should be apparent to those skilled in the art that in certain cases, the compound of Formula II may exist as a mixture of tautomers. The present disclosure is not limited to any specific tautomer. Rather, the present disclosure encompasses any and all of such tautomers whether or not explicitly drawn or referred to.

In some embodiments, the compound of Formula II (including any of the applicable sub-formulae as described herein) can exist as an isotopically labeled compound, particularly, a deuterated analog, wherein one or more of the hydrogen atoms of the compound of Formula II is/are substituted with a deuterium atom with an abundance above its natural abundance, e.g., a CD₃ analog when the compound has a CH₃ group.

Typically, X in Formula II is N, and the compound of Formula II can be characterized as having Formula II-A:

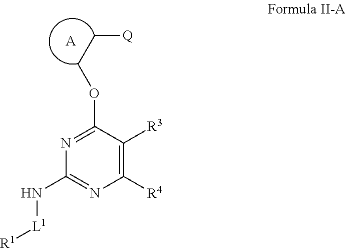

wherein L¹, R¹, Ring A, Q, R³, and R⁴ include any of those described herein in any combination. For example, the variables L¹, R¹, R³, and R⁴ can include any of those defined herein in connection with Formula I in any combination.

Various ring structures are suitable as Ring A in Formula II. For example, in some embodiments, Ring A is an optionally substituted C₄₋₁₀ cycloalkyl or optionally substituted 4-10 membered heterocyclic ring having 1-4 ring heteroatoms independently selected from O, S, and N. Ring A can be monocyclic or polycyclic, which can include a fused, bridged, or spiro ring structure. For example, in some embodiments, Ring A can be an optionally substituted monocyclic C₄-s cycloalkyl such as C₄, C₅, C₆, or C₇ cycloalkyl. In some embodiments, Ring A is an optionally substituted fused, bridged, or spiro bicyclic C₆₋₁₀ cycloalkyl, e.g., described herein. In some embodiments, Ring A can be an optionally substituted monocyclic 4-8 membered heterocyclic ring, for example, those having one ring heteroatom selected from O and N. In some embodiments, Ring A is an optionally substituted fused, bridged, or spiro bicyclic 6-10 membered heterocyclic ring, for example, those having one or two ring heteroatoms independently selected from O, S, and N. When further substituted, Ring A can be typically substituted with 1-3 substituents, each independently selected from oxo, halogen (e.g., F), CN, G¹, C(O)H, C(O)G¹, OH, O-G¹, NH₂, NH(G¹), and N(G¹)(G¹), wherein G¹ at each occurrence is independently a C₁₋₄ alkyl optionally substituted with 1-3 substituents independently selected from F, CN, OH, and C₁₋₄ heteroalkyl, or a C₃₋₆ cycloalkyl optionally substituted with 1-3 substituents independently selected from F, CN, OH, and C₁₋₄heteroalkyl. In some embodiments, Ring A can also be deuterated, for example, with one or more ring CH₂ groups replaced with CD₂ groups.

Various groups are suitable as Q for Formula II. In some embodiments, Q is OR^{A}. For example, in some embodiments, Q is OR^{A}, wherein R^{A} is hydrogen, C₁₋₄ alkyl optionally substituted with 1-3 substituents independently selected from F, CN, OH, and C₁₋₄ heteroalkyl, or a C₃₋₆ cycloalkyl optionally substituted with 1-3 substituents independently selected from F, CN, OH, and C₁₋₄ heteroalkyl. In some preferred embodiments, Q in Formula II (e.g., any of the applicable subformulae) is OH.

In some embodiments, Q can be an optionally substituted C₁₋₄ alkyl, such as fluorine substituted C₁₋₄ alkyl or hydroxyl substituted C₁₋₄ alkyl, for example, CH₂OH.

In some embodiments, Q can be a halogen, such as F, or a CN. In some embodiments, Q can also be COR^{B}. For example, in some embodiments, Q is COR^{B}, wherein R^{B} is hydrogen, C₁₋₄ alkyl optionally substituted with 1-3 substituents independently selected from F, CN, OH, and C₁₋₄ heteroalkyl, or a C₃₋₆ cycloalkyl optionally substituted with 1-3 substituents independently selected from F, CN, OH, and C₁₋₄ heteroalkyl.

In some embodiments, Q can be F, CN, C(O)H, C(O)—(C₁₋₄ alkyl optionally substituted with F), CH₂OH, C₁₋₄ alkyl optionally substituted with F, or C₁₋₄ alkoxy optionally substituted with F.

In some embodiments,

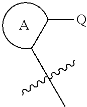

in Formula II (e.g., II-A) can be selected from:

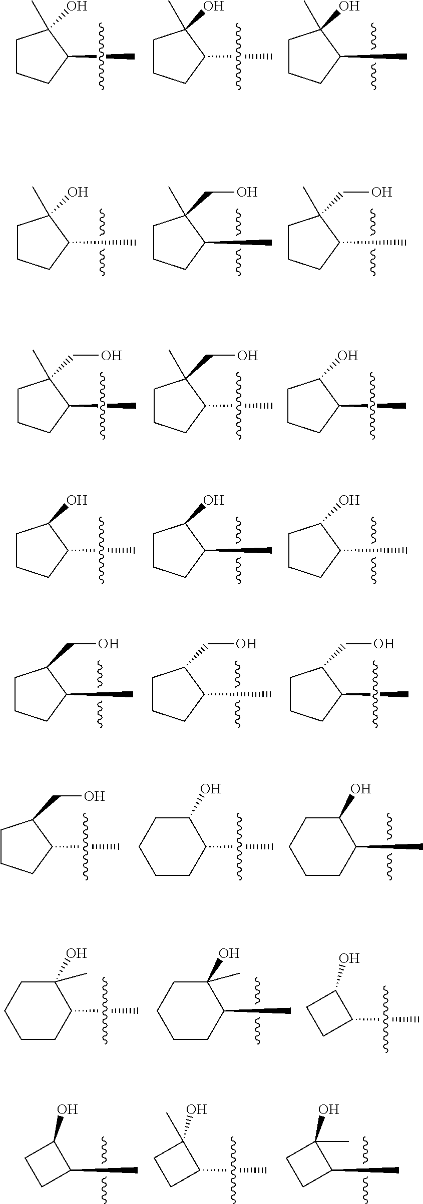

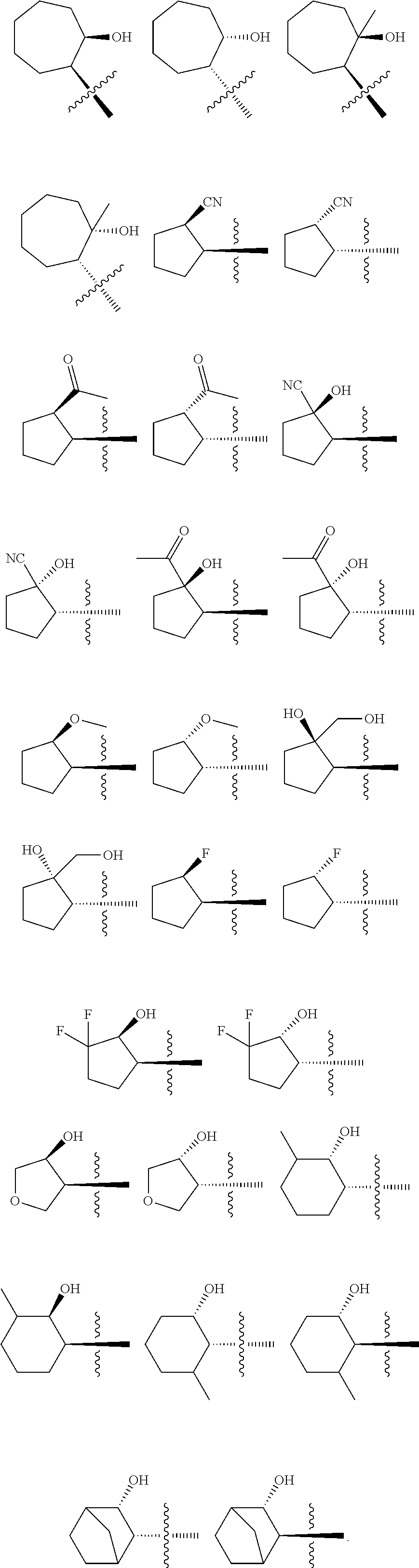

In some embodiments,

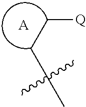

in Formula II (e.g., II-A) can be selected from:

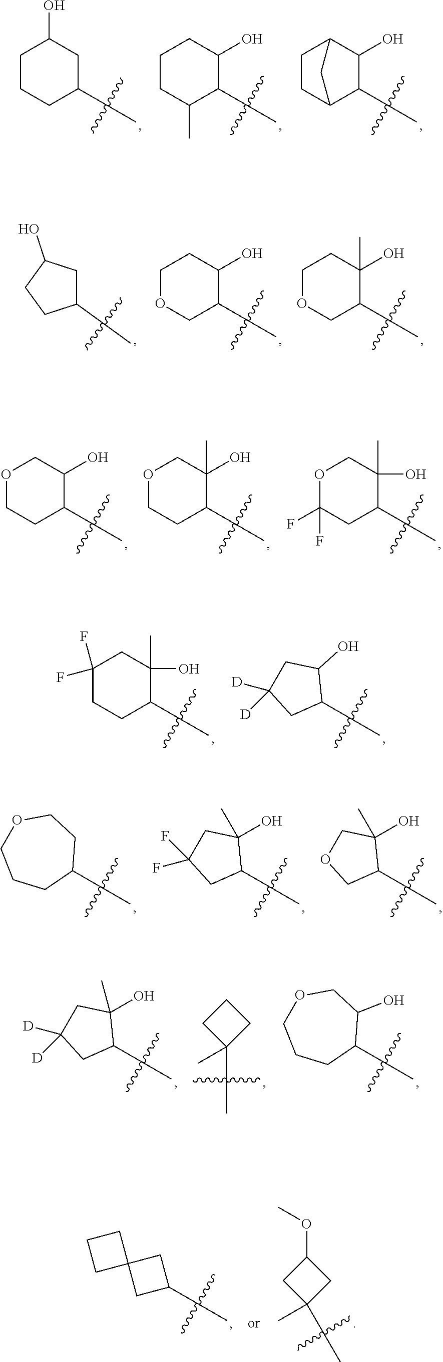

In some preferred embodiments,

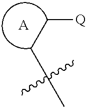

in Formula II (e.g., II-A) can be selected from:

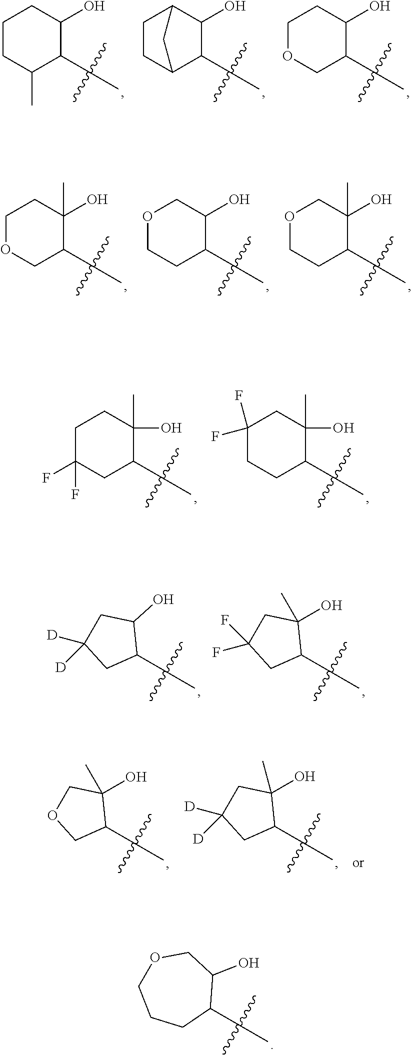

In some preferred embodiments,

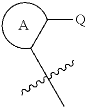

in Formula II (e.g., II-A) can be selected from:

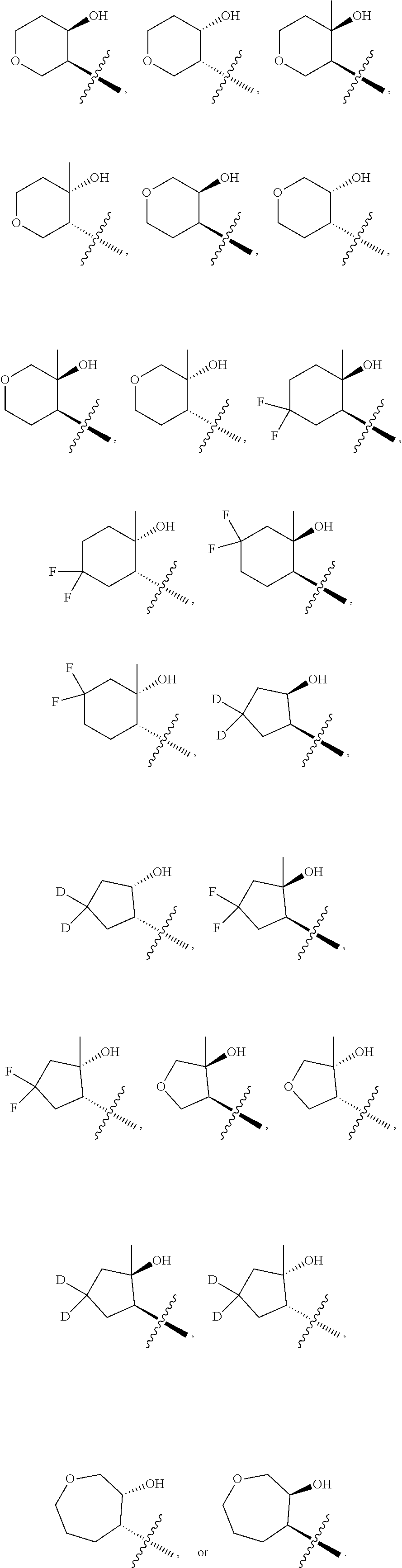

In some embodiments, the compound of Formula II can be characterized as having a subformula of Formula II-1 or II-2, or a deuterated analog thereof:

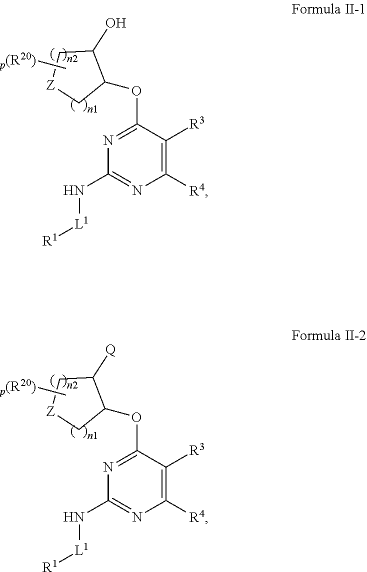

wherein:
n1 and n2 are independently 0, 1, 2, or 3,
Z is CR²¹R²², O, or NR²³,
p is 0, 1, 2, 3, or 4, as valency permits,
R²⁰ at each occurrence is independently oxo, halogen (e.g., F), CN, G¹, C(O)H, C(O)G¹, OH, O-G¹, NH₂, NH(G¹), and N(G¹)(G¹), wherein G¹ at each occurrence is independently a C₁₋₄ alkyl optionally substituted with 1-3 substituents independently selected from F, CN, OH, and C₁₋₄ heteroalkyl, or a C₃₋₆ cycloalkyl optionally substituted with 1-3 substituents independently selected from F, CN, OH, and C₁₋₄ heteroalkyl,
or two geminal R²⁰ form an oxo group, or two R²⁰ together with the intervening atoms form an optionally substituted ring structure,
R²¹ and R²² are each independently hydrogen or R²⁰,
or R²¹ and R²² together form an oxo group or an optionally substituted ring structure, or one of R²¹ and R²² with one R²⁰ group together with the intervening atoms form an optionally substituted ring structure,
R²³ is hydrogen or R²⁰
or R²³ and one R²⁰ group together with the intervening atoms form an optionally substituted ring structure,
wherein Q, L¹, R¹, R³ and R⁴ include any of those described herein in any combination. To be clear, the variables R²¹, R²², and R²³, although can have the same definition as R²⁰ do not count towards the number of R²⁰ groups as drawn in Formula II-1 or II-2. In other words, the integer p refers to potential substitutions of the ring at any available position other than the Z group.

Typically, n2 in Formula II-1 or II-2 is 1.

Typically, n1 in Formula II-1 or II-2 is 0, 1, or 2.

In some embodiments, n1 and n2 are such that the ring is a 4-8 membered ring, such as a 4, 5, 6, or 7 membered ring.

In some embodiments, Z in Formula II-1 or II-2 is CH₂, O, or NR²³, wherein R²³ is hydrogen or a C₁₋₄ alkyl optionally substituted with 1-3 substituents independently selected from F, CN, and OH.

In some preferred embodiments, Z in Formula II-1 or II-2 is CH₂.

In some preferred embodiments, Z in Formula II-1 or II-2 is CF₂.

Compounds of Formula II-1 or II-2 can exist in a deuterated form. For example, in some preferred embodiments, the hydrogens on Z group can be replaced with deuterium, in other words, the Z group in Formula II-1 or II-2 can be CD₂.

In some preferred embodiments, Z in Formula II-1 or II-2 is O.

The integer p in Formula II-1 or II-2 is typically 0-2. For example, in some embodiments, p in Formula II-1 or II-2 is 0. In some embodiments, p in Formula II-1 or II-2 is 1 or 2.

In some embodiments, p in Formula II-1 or II-2 is 1 or 2, R²⁰ at each occurrence is independently halogen (e.g., F), CN, G¹, C(O)H, C(O)G¹, OH, or O-G¹. For example, in some embodiments, p in Formula II-1 or II-2 is 1 or 2, R²⁰ at each occurrence is independently halogen (e.g., F), CN, G¹, C(O)H, C(O)G¹, OH, or O-G¹, wherein G¹ is a C₁₋₄ alkyl optionally substituted with 1-3 F.

Various groups are suitable for use as Q in Formula II-2, which includes any of the definition of Q as described herein. In some embodiments, Q in Formula II-2 can be F, CN, C(O)H, C(O)—(C₁₋₄ alkyl optionally substituted with F), CH₂OH, C₁₋₄ alkyl optionally substituted with F, or C₁₋₄ alkoxy optionally substituted with F.

In some preferred embodiments, the

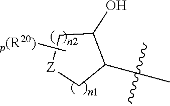

moiety in Formula II-1 can be selected from:

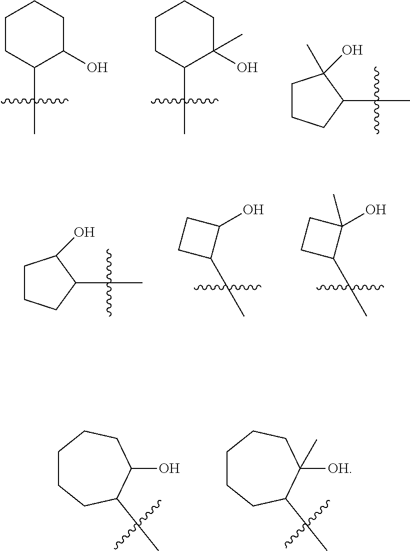

In some preferred embodiments, the

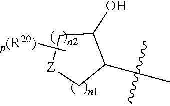

moiety in Formula II-1 can be selected from:

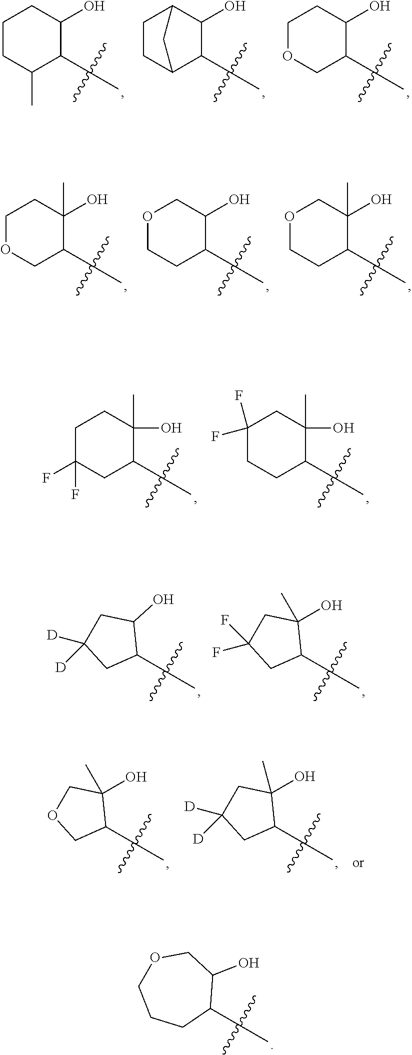

In some preferred embodiments, the

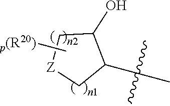

moiety in Formula II-1 is

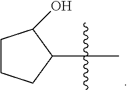 [SMILES: *C1CCCC1O]

In some preferred embodiments, the

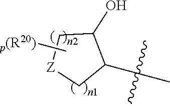

moiety in Formula II-1 is

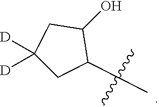 [SMILES: *C1CC([2H])([2H])CC1O]

In some preferred embodiments, the

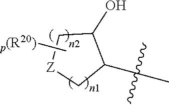

moiety in Formula II-1

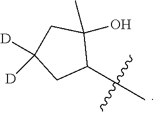 [SMILES: *C1CC([2H])([2H])CC1(C)O]

In some preferred embodiments, the

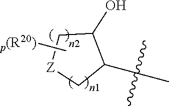

moiety in Formula II-1 is

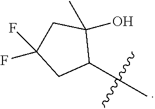 [SMILES: *C1CC(F)(F)CC1(C)O]

In some preferred embodiments, the

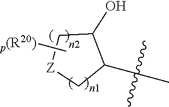

moiety in Formula II-1 is

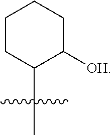 [SMILES: *C1CCCCC1O]

In some preferred embodiments, the

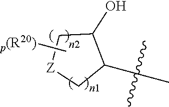

moiety in Formula II-1 is

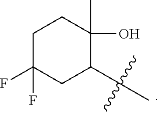 [SMILES: *C1CC(F)(F)CCC1(C)O]

In some preferred embodiments, the

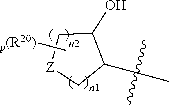

moiety in Formula II-1 is

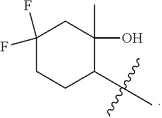 [SMILES: *C1CCC(F)(F)CC1(C)O]

In some preferred embodiments, the

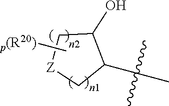

moiety in Formula II-1 is

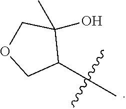 [SMILES: *C1COCC1(C)O]

In some preferred embodiments, the

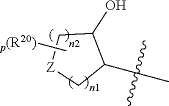

moiety

### in Formula II-1 is

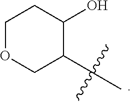 [SMILES: *C1COCCC1O]

In some embodiments, the

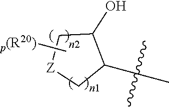

moiety in Formula II-1 is

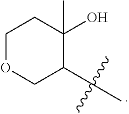 [SMILES: *C1COCCC1(C)O ; "."]

In some preferred embodiments, the

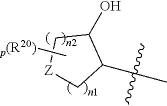

moiety in Formula II-1 is

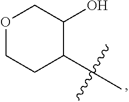 [SMILES: *C1CCOCC1O ; ","]

In some embodiments, the

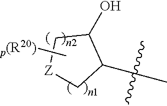

moiety in Formula II-1 is

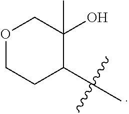 [SMILES: *C1CCOCC1(C)O ; "."]

In some embodiments, the

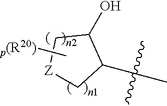

moiety in Formula II-1 is

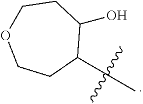 [SMILES: *C1CCOCCC1O ; "."]

Compounds of Formula II-1 or II-2 can exist in various stereoisomeric forms, such as in racemic forms, substantially pure individual stereoisomers, a mixture enriched in one or more stereoisomers, or a mixture of stereoisomers in any ratio. For example, in some embodiments, the compound of Formula II-1 can be characterized as having Formula II-1-S1, II-1-S2, II-1-S3, or II-1-S4:

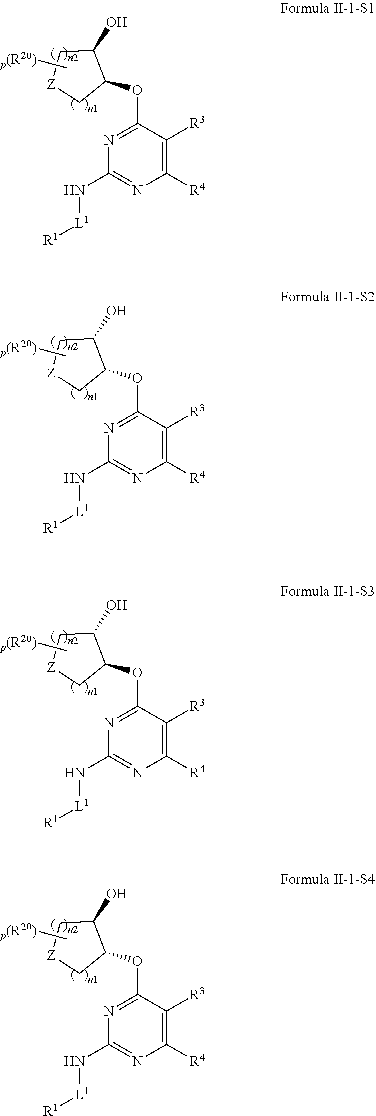

wherein the variable n1, n2, Z, R²⁰, p, L¹, R¹, R³, and R⁴ include any of those described herein in any combination. In some embodiments, the compound of any of Formula II-1-S1, II-1-S2, II-1-S3, or II-1-S4 can exist as a substantially pure stereoisomer (the respective as-drawn stereoisomer), for example, substantially free (e.g., with less than 10%, less than 5%, less than 1%, by weight and/or by HPLC or SFC area, or non-detectable amount) of the other potential stereoisomers. For example, in some embodiments, the compound of Formula II-1-S1 can be a substantially pure stereoisomer, wherein out of the four potential stereoisomers, the combined amount of the corresponding stereoisomers of Formula II-1-S2, II-1-S3, and II-1-S4 that may be present is less than 10%, less than 5%, less than 1%, by weight and/or by HPLC or SFC area, or in a non-detectable amount. In some embodiments, the compound of Formula II-1 can also exist as a mixture of any two or more of the corresponding Formula II-1-S1, II-1-S2, II-1-S3, or II-1-S4 in any ratio, such as a racemic mixture of II-1-S1 and II-1-S2 or a racemic mixture of II-1-S3 and II-1-S4. Exemplary methods for separating the stereoisomers are shown herein in the Examples section. In some preferred embodiments, the compound of Formula II-1 can be characterized as being a cis isomer, which can exist in the corresponding stereoisomer of Formula II-1-S1 or II-1-S2, or a mixture thereof in any ratio, such as a racemic mixture or a mixture enriched in the stereoisomer of Formula II-1-S1 or II-1-S2, such as having an enantiomeric excess of about 50% or higher, such as about 80% or higher, about 90% or higher, about 95% or higher.

In some embodiments, the compound of Formula II-2 can be characterized as having Formula II-2-S1, II-2-S2, II-2-S3, or II-2-S4:

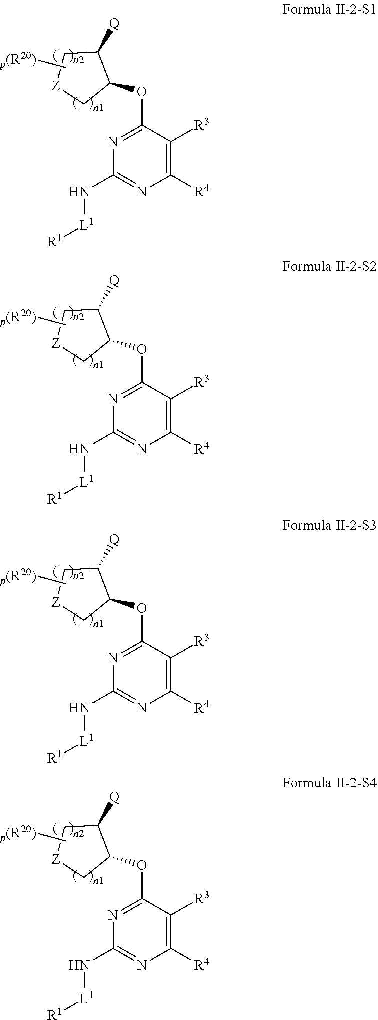

wherein the variables n1, n2, Z, R, p, Q, L¹, R¹, R³, and R⁴ include any of those described herein in any combination. In some embodiments, the compound of any of Formula II-2-S1, II-2-S2, II-2-S3, or II-2-S4 can exist as a substantially pure stereoisomer (the respective as-drawn stereoisomer), for example, substantially free (e.g., with less than 10%, less than 5%, less than 1%, by weight and/or by HPLC or SFC area, or non-detectable amount) of the other potential stereoisomers. For example, in some embodiments, the compound of Formula II-2-S1 can be a substantially pure stereoisomer, wherein out of the four potential stereoisomers, the combined amount of the corresponding stereoisomers of Formula II-2-S2, II-2-S3, and II-2-S4 that may be present is less than 10%, less than 5%, less than 1%, by weight and/or by HPLC or SFC area, or in a non-detectable amount. In some embodiments, the compound of Formula II-2 can also exist as a mixture of any two or more of the corresponding Formula II-2-S1, II-2-S2, II-2-S3, or II-2-S4 in any ratio, such as a racemic mixture of II-2-S1 and II-2-S2 or a racemic mixture of II-2-S3 and II-2-S4. Exemplary methods for separating the stereoisomers are shown herein in the Examples section. In some preferred embodiments, the compound of Formula II-2 can be characterized as being a cis isomer, which can exist in the corresponding stereoisomer of Formula II-2-S1 or II-2-S2, or a mixture thereof in any ratio, such as a racemic mixture or a mixture enriched in the stereoisomer of Formula II-2-S1 or II-2-S2, such as having an enantiomeric excess of about 50% or higher, such as about 80% or higher, about 90% or higher, about 95% or higher.

The variable L¹, R¹, R³, and R⁴ for Formula II and any of the applicable subformulae include any of those described herein in any combination, which also includes any of those described herein in connection with Formula I and its subformulae. For example, in some embodiments, L¹-R¹ in Formula II (e.g., II-A, II-1, or II-2) can be selected from:

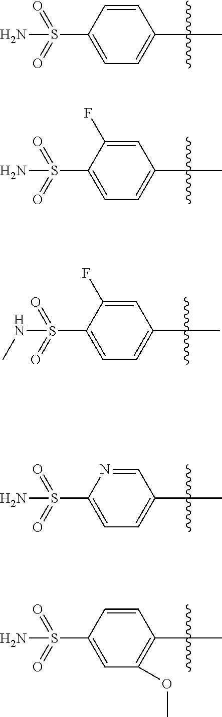 [SMILES: *c1ccc(S(N)(=O)=O)cc1 ; *c1ccc(S(N)(=O)=O)c(F)c1 ; *c1ccc(S(=O)(=O)NC)c(F)c1 ; *c1ccc(S(N)(=O)=O)nc1 ; *c1ccc(S(N)(=O)=O)cc1OC]

or L¹-R¹ is

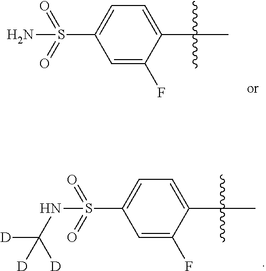 [SMILES: *c1ccc(S(N)(=O)=O)cc1F ; *c1ccc(S(=O)(=O)NC([2H])([2H])[2H])cc1F]

In some embodiments, L¹-R¹ in Formula II (e.g., II-A, II-1, or II-2) is selected from:

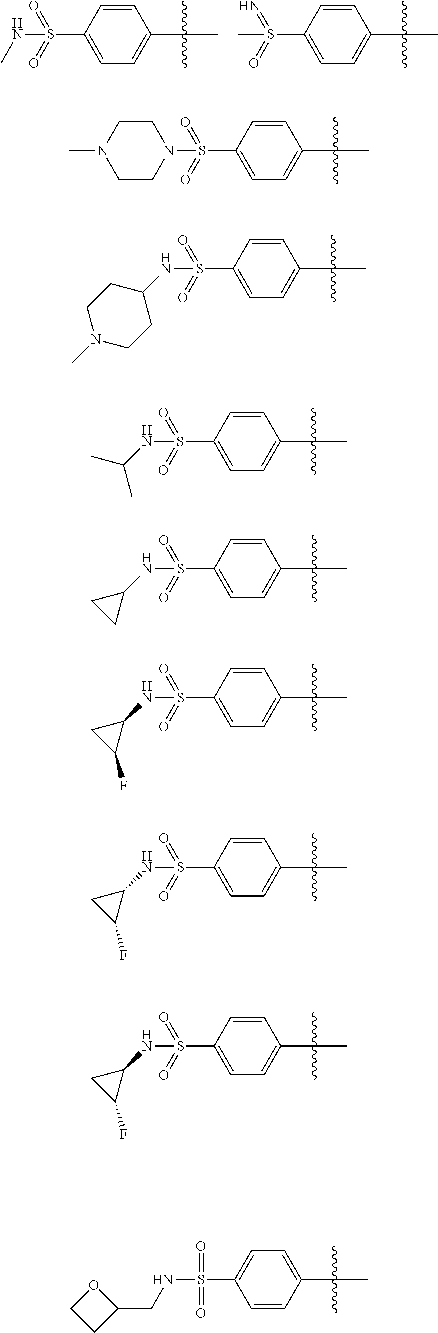

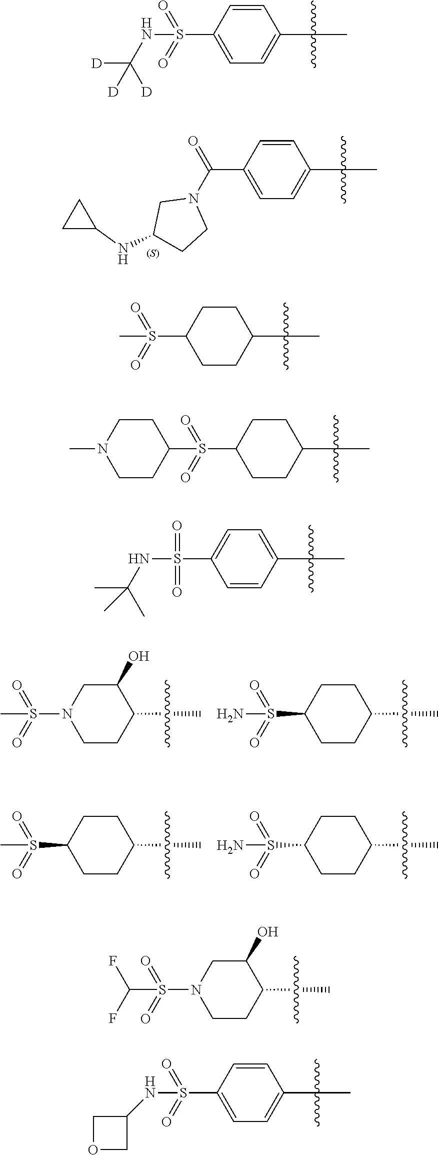

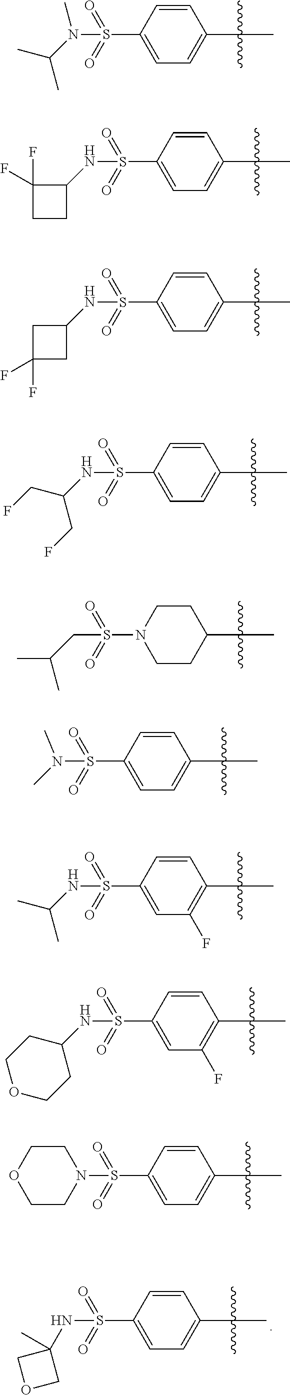

In some embodiments, L¹-R¹ in Formula II (e.g., II-A, II-1, or II-2) is selected from:

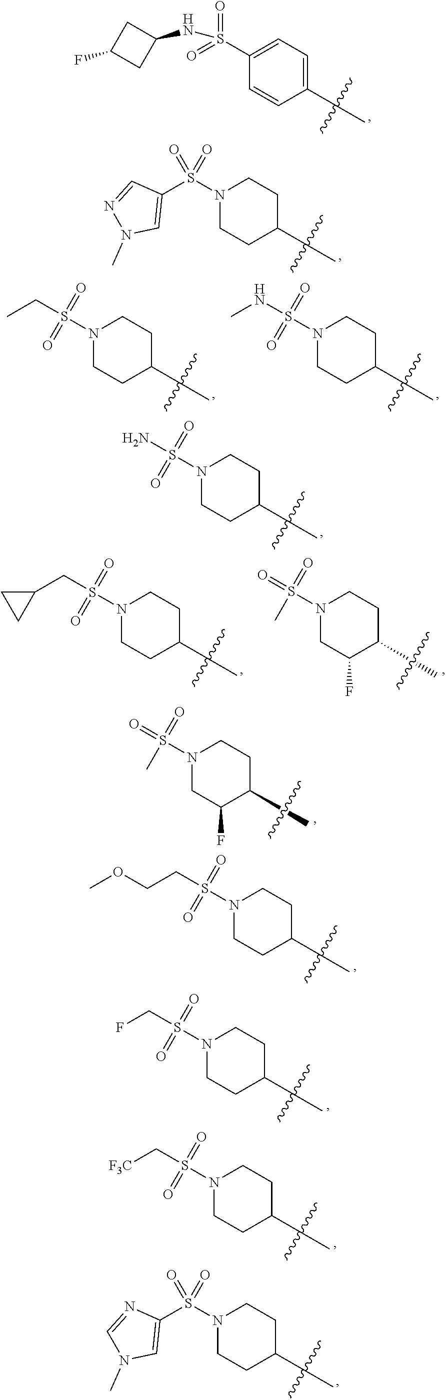

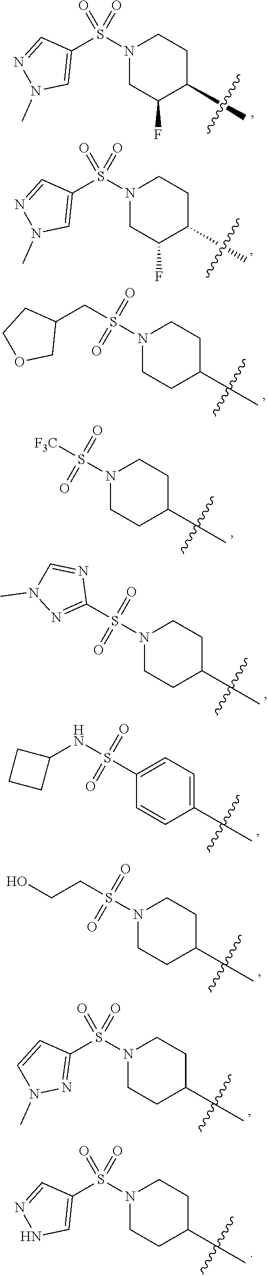

In some embodiments, L¹-R¹ in Formula II (e.g., II-A, II-1, or II-2) is selected from:

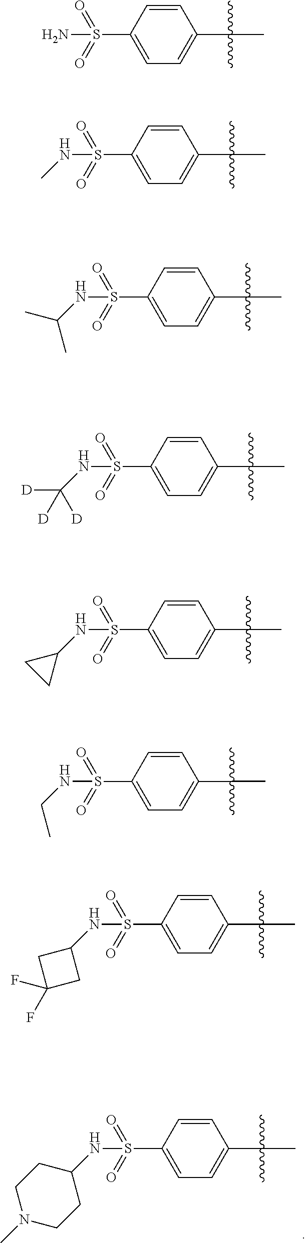

In some embodiments, R³ in Formula II (e.g., II-A, II-1, or II-2) is hydrogen, F, Cl, Br, C₁₋₄ alkyl optionally substituted with deuterium and/or F, or CN. For example, in some embodiments, R³ in Formula II (e.g., II-A, II-1, or II-2) can be a C₁₋₄ alkyl optionally substituted with 1-3 F, such as methyl, CD₃, ethyl, CHF₂, CF₂CH₃, CH₂CH₂F, CH₂CF₂H, or CF₃. In some embodiments, R³ in Formula II (e.g., II-A, II-1, or II-2) can be methyl, CD₃, CH₂-OMe, CH₂—OCD₃, ethyl, CHF₂, CF₂CH₃, CH₂CH₂F, CH₂CF₂H, or CF₃. In some embodiments, R³ in Formula II (e.g., II-A, II-1, or II-2) is OR^{A}, wherein R^{A} is defined herein, for example, R^{A} is hydrogen, C₁₋₄ alkyl optionally substituted with 1-3 substituents independently selected from deuterium, F, CN, OH, and C₁₋₄ heteroalkyl, or a C₃₋₆ cycloalkyl optionally substituted with 1-3 substituents independently selected from deuterium, F, CN, OH, and C₁₋₄ heteroalkyl. In some embodiments, R³ in Formula II (e.g., II-A, II-1, or II-2) is C(O)R^{B}, wherein R^{B} is defined herein, for example, R^{B} is hydrogen, C₁₋₄ alkyl optionally substituted with 1-3 substituents independently selected from deuterium, F, CN, OH, and C₁₋₄ heteroalkyl, or a C₃₋₆ cycloalkyl optionally substituted with 1-3 substituents independently selected from deuterium, F, CN, OH, and C₁₋₄ heteroalkyl. In some embodiments, R³ in Formula II (e.g., II-A, II-1, or II-2) is selected from

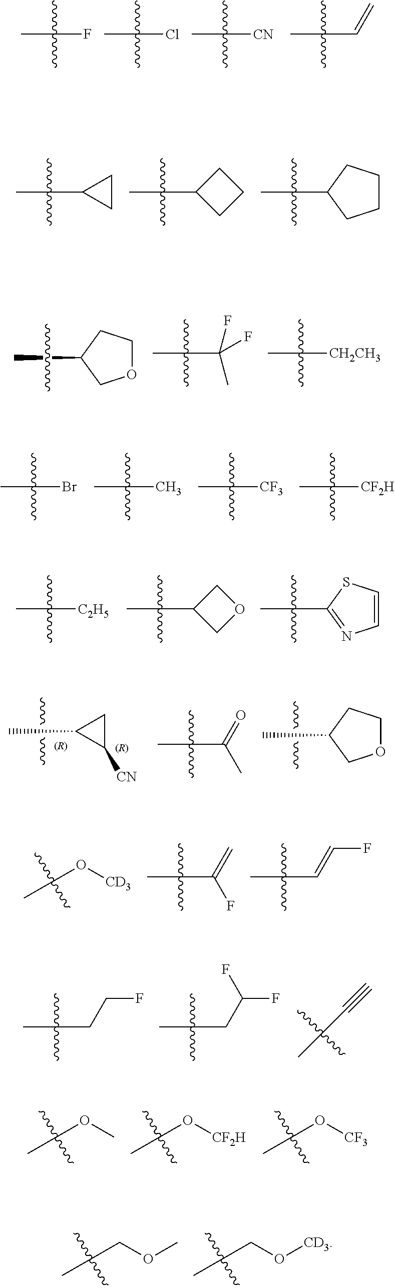

In some preferred embodiments, R³ in Formula II (e.g., II-A, II-1, or II-2) is CN. In some preferred embodiments, R³ in Formula II (e.g., II-A, II-1, or II-2) is F, Cl, or Br. In some preferred embodiments, R³ in Formula II (e.g., II-A, II-1, or II-2) is CF₃. In some preferred embodiments, R³ in Formula II (e.g., II-A, II-1, or II-2) is methyl or ethyl. In some preferred embodiments, R³ in Formula II (e.g., II-A, II-1, or II-2) is CHF₂, CF₂CH₃, CH₂CH₂F, or CH₂CF₂H. In some preferred embodiments, R³ in Formula II (e.g., II-A, II-1, or II-2) is cyclopropyl. In some preferred embodiments, R³ in Formula II (e.g., II-A, II-1, or II-2) is

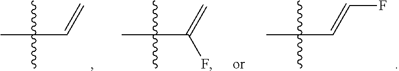

In some preferred embodiments, R³ in Formula II (e.g., II-A, II-1, or II-2) is

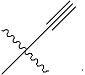 [SMILES: *C#C]

Typically, R⁴ in Formula II (e.g., II-A, II-1, or II-2) is hydrogen. In some embodiments, R⁴ can be NH₂. In some embodiments, R³ and R⁴ in Formula II (e.g., II-A, II-1, or II-2) can be joined to form a 5- or 6-membered heteroaryl structure, which has 1-3 ring heteroatoms independently selected from N, O, and S, which is optionally substituted with one or more (e.g., 1, 2, or 3) substituents independently selected from F, CN, OH, and 4-6 membered heterocyclyl having 1-2 ring heteroatoms independently selected from N, O, and S, which is optionally substituted with 1-3 substituents independently selected from oxo, F, CN, and OH. For example, in some embodiments, R³ and R⁴ are joined to form

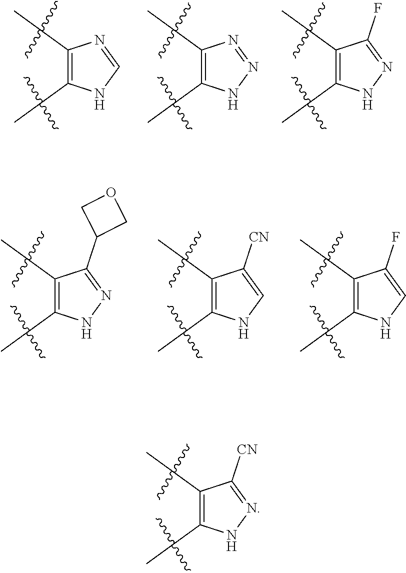

In some embodiments, the present disclosure also provide a compound selected from Table 1A or Table 1B below, a deuterated analog thereof, a stereoisomer thereof, or a pharmaceutically acceptable salt thereof:

TABLE 1A
List of Compounds

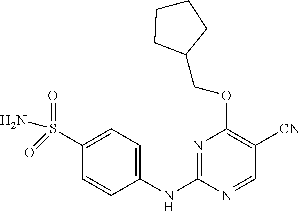 [SMILES: N#Cc1cnc(Nc2ccc(S(N)(=O)=O)cc2)nc1OCC1CCCC1]

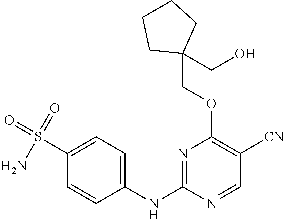 [SMILES: N#Cc1cnc(Nc2ccc(S(N)(=O)=O)cc2)nc1OCC1(CO)CCCC1]

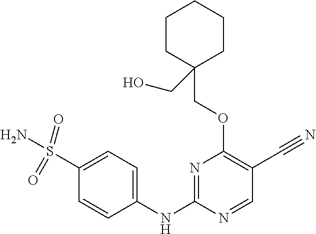 [SMILES: N#Cc1cnc(Nc2ccc(S(N)(=O)=O)cc2)nc1OCC1(CO)CCCCC1]

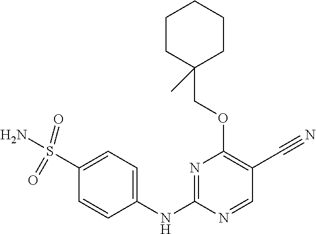 [SMILES: CC1(COc2nc(Nc3ccc(S(N)(=O)=O)cc3)ncc2C#N)CCCCC1]

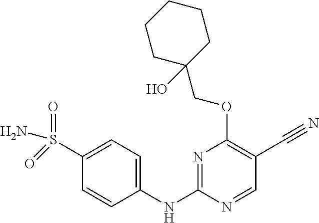 [SMILES: N#Cc1cnc(Nc2ccc(S(N)(=O)=O)cc2)nc1OCC1(O)CCCCC1]

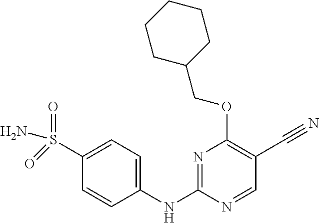 [SMILES: N#Cc1cnc(Nc2ccc(S(N)(=O)=O)cc2)nc1OCC1CCCCC1]

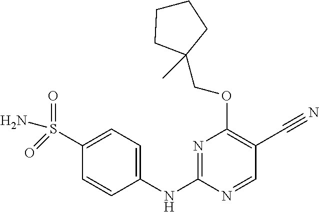 [SMILES: CC1(COc2nc(Nc3ccc(S(N)(=O)=O)cc3)ncc2C#N)CCCC1]

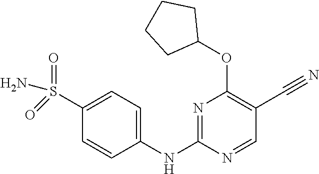 [SMILES: N#Cc1cnc(Nc2ccc(S(N)(=O)=O)cc2)nc1OC1CCCC1]

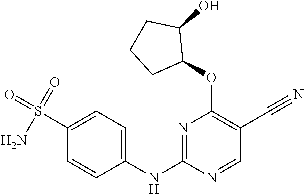 [SMILES: N#Cc1cnc(Nc2ccc(S(N)(=O)=O)cc2)nc1O[C@H]1CCC[C@H]1O]

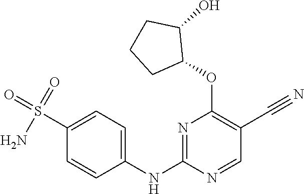 [SMILES: N#Cc1cnc(Nc2ccc(S(N)(=O)=O)cc2)nc1O[C@@H]1CCC[C@@H]1O]

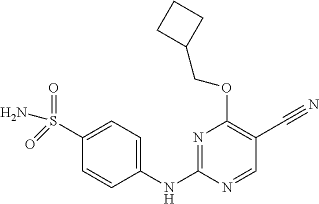 [SMILES: N#Cc1cnc(Nc2ccc(S(N)(=O)=O)cc2)nc1OCC1CCC1]

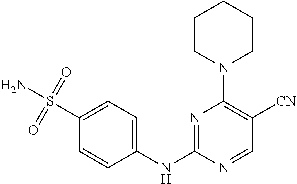 [SMILES: N#Cc1cnc(Nc2ccc(S(N)(=O)=O)cc2)nc1N1CCCCC1]

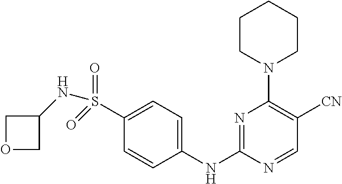 [SMILES: N#Cc1cnc(Nc2ccc(S(=O)(=O)NC3COC3)cc2)nc1N1CCCCC1]

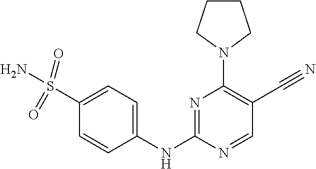 [SMILES: N#Cc1cnc(Nc2ccc(S(N)(=O)=O)cc2)nc1N1CCCC1]

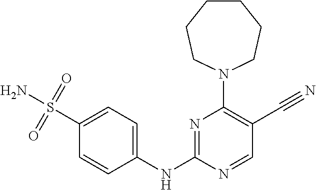 [SMILES: N#Cc1cnc(Nc2ccc(S(N)(=O)=O)cc2)nc1N1CCCCCC1]

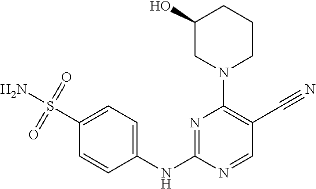 [SMILES: N#Cc1cnc(Nc2ccc(S(N)(=O)=O)cc2)nc1N1CCC[C@H](O)C1]

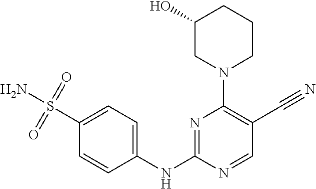 [SMILES: N#Cc1cnc(Nc2ccc(S(N)(=O)=O)cc2)nc1N1CCC[C@@H](O)C1]

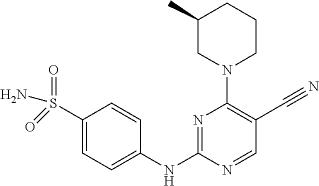 [SMILES: C[C@H]1CCCN(c2nc(Nc3ccc(S(N)(=O)=O)cc3)ncc2C#N)C1]

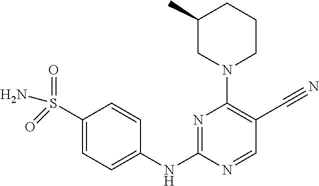 [SMILES: C[C@H]1CCCN(c2nc(Nc3ccc(S(N)(=O)=O)cc3)ncc2C#N)C1]

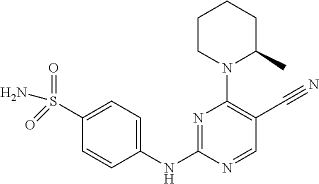 [SMILES: C[C@@H]1CCCCN1c1nc(Nc2ccc(S(N)(=O)=O)cc2)ncc1C#N]

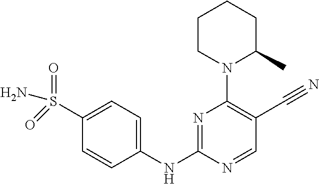 [SMILES: C[C@@H]1CCCCN1c1nc(Nc2ccc(S(N)(=O)=O)cc2)ncc1C#N]

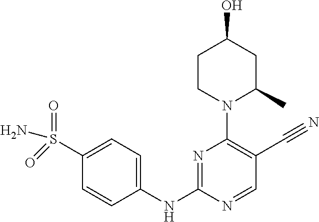 [SMILES: C[C@@H]1C[C@H](O)CCN1c1nc(Nc2ccc(S(N)(=O)=O)cc2)ncc1C#N]

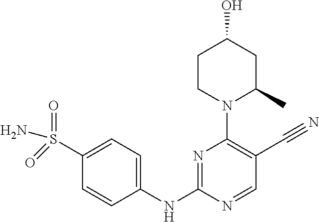 [SMILES: C[C@@H]1C[C@@H](O)CCN1c1nc(Nc2ccc(S(N)(=O)=O)cc2)ncc1C#N]

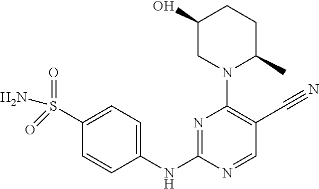 [SMILES: C[C@@H]1CC[C@H](O)CN1c1nc(Nc2ccc(S(N)(=O)=O)cc2)ncc1C#N]

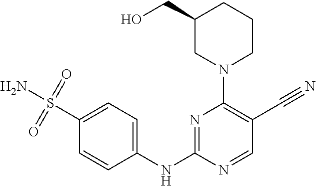 [SMILES: N#Cc1cnc(Nc2ccc(S(N)(=O)=O)cc2)nc1N1CCC[C@H](CO)C1]

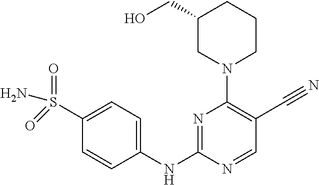 [SMILES: N#Cc1cnc(Nc2ccc(S(N)(=O)=O)cc2)nc1N1CCC[C@@H](CO)C1]

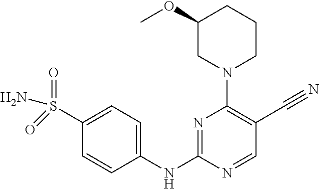 [SMILES: CO[C@H]1CCCN(c2nc(Nc3ccc(S(N)(=O)=O)cc3)ncc2C#N)C1]

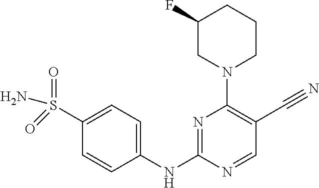 [SMILES: N#Cc1cnc(Nc2ccc(S(N)(=O)=O)cc2)nc1N1CCC[C@H](F)C1]

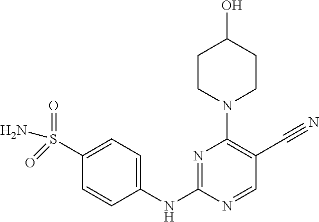 [SMILES: N#Cc1cnc(Nc2ccc(S(N)(=O)=O)cc2)nc1N1CCC(O)CC1]

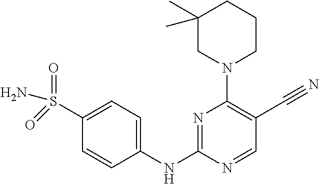 [SMILES: CC1(C)CCCN(c2nc(Nc3ccc(S(N)(=O)=O)cc3)ncc2C#N)C1]

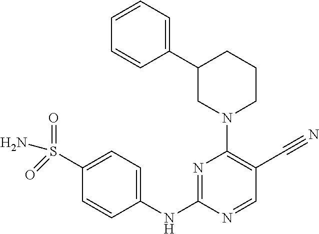 [SMILES: N#Cc1cnc(Nc2ccc(S(N)(=O)=O)cc2)nc1N1CCCC(c2ccccc2)C1]

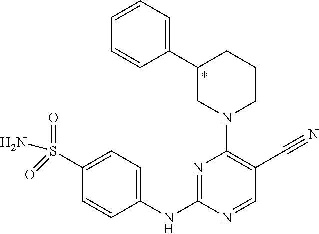 [SMILES: N#Cc1cnc(Nc2ccc(S(N)(=O)=O)cc2)nc1N1CCCC(c2ccccc2)C1]

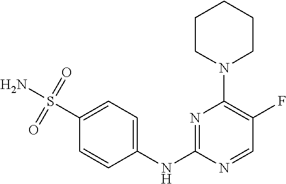 [SMILES: NS(=O)(=O)c1ccc(Nc2ncc(F)c(N3CCCCC3)n2)cc1]

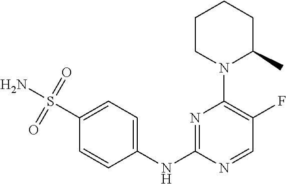 [SMILES: C[C@@H]1CCCCN1c1nc(Nc2ccc(S(N)(=O)=O)cc2)ncc1F]

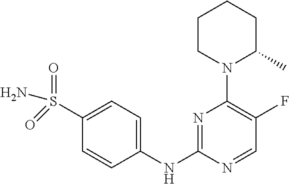 [SMILES: C[C@H]1CCCCN1c1nc(Nc2ccc(S(N)(=O)=O)cc2)ncc1F]

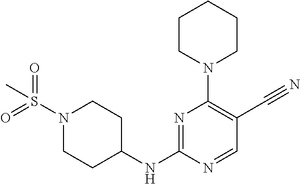 [SMILES: CS(=O)(=O)N1CCC(Nc2ncc(C#N)c(N3CCCCC3)n2)CC1]

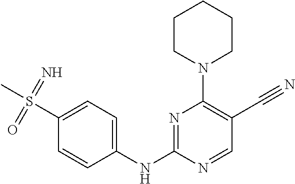 [SMILES: CS(=N)(=O)c1ccc(Nc2ncc(C#N)c(N3CCCCC3)n2)cc1]

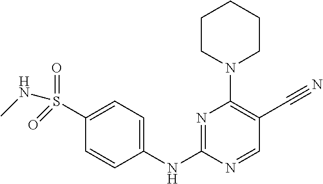 [SMILES: CNS(=O)(=O)c1ccc(Nc2ncc(C#N)c(N3CCCCC3)n2)cc1]

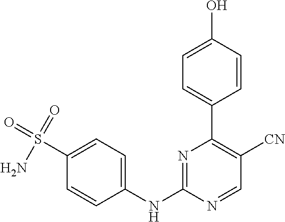 [SMILES: N#Cc1cnc(Nc2ccc(S(N)(=O)=O)cc2)nc1-c1ccc(O)cc1]

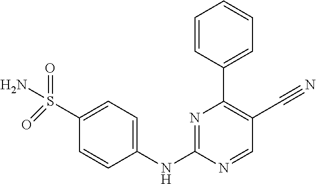 [SMILES: N#Cc1cnc(Nc2ccc(S(N)(=O)=O)cc2)nc1-c1ccccc1]

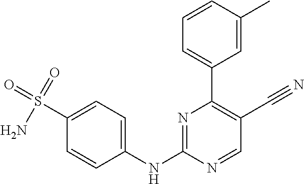 [SMILES: Cc1cccc(-c2nc(Nc3ccc(S(N)(=O)=O)cc3)ncc2C#N)c1]

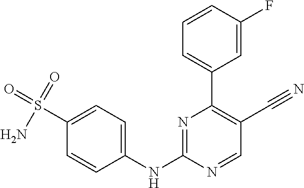 [SMILES: N#Cc1cnc(Nc2ccc(S(N)(=O)=O)cc2)nc1-c1cccc(F)c1]

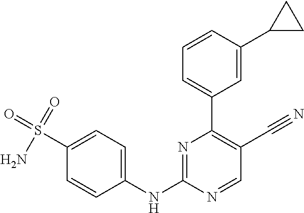 [SMILES: N#Cc1cnc(Nc2ccc(S(N)(=O)=O)cc2)nc1-c1cccc(C2CC2)c1]

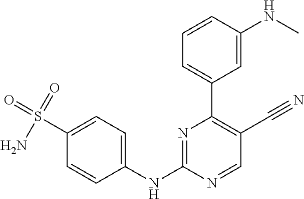 [SMILES: CNc1cccc(-c2nc(Nc3ccc(S(N)(=O)=O)cc3)ncc2C#N)c1]

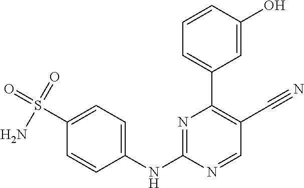 [SMILES: N#Cc1cnc(Nc2ccc(S(N)(=O)=O)cc2)nc1-c1cccc(O)c1]

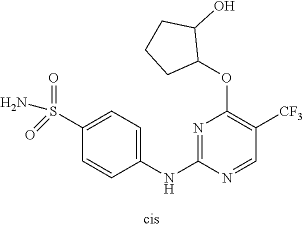 [SMILES: NS(=O)(=O)c1ccc(Nc2ncc(C(F)(F)F)c(OC3CCCC3O)n2)cc1]

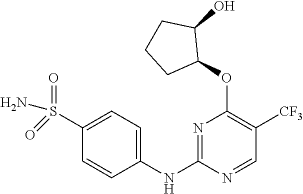 [SMILES: NS(=O)(=O)c1ccc(Nc2ncc(C(F)(F)F)c(O[C@H]3CCC[C@H]3O)n2)cc1]

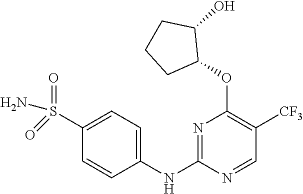 [SMILES: NS(=O)(=O)c1ccc(Nc2ncc(C(F)(F)F)c(O[C@@H]3CCC[C@@H]3O)n2)cc1]

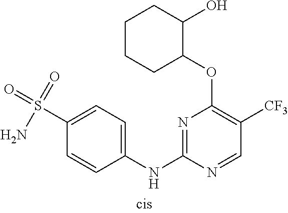 [SMILES: NS(=O)(=O)c1ccc(Nc2ncc(C(F)(F)F)c(OC3CCCCC3O)n2)cc1]

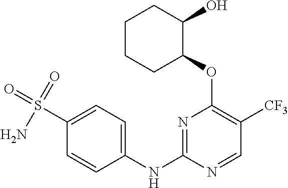 [SMILES: NS(=O)(=O)c1ccc(Nc2ncc(C(F)(F)F)c(O[C@H]3CCCC[C@H]3O)n2)cc1]

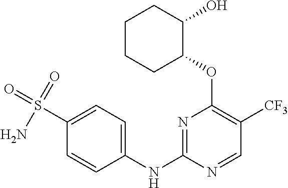 [SMILES: NS(=O)(=O)c1ccc(Nc2ncc(C(F)(F)F)c(O[C@@H]3CCCC[C@@H]3O)n2)cc1]

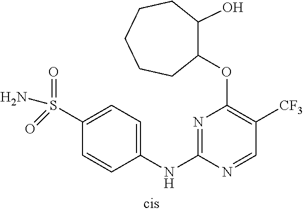 [SMILES: NS(=O)(=O)c1ccc(Nc2ncc(C(F)(F)F)c(OC3CCCCCC3O)n2)cc1]

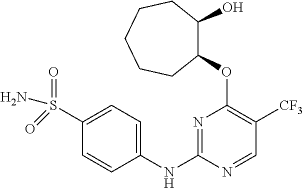 [SMILES: NS(=O)(=O)c1ccc(Nc2ncc(C(F)(F)F)c(O[C@H]3CCCCC[C@H]3O)n2)cc1]

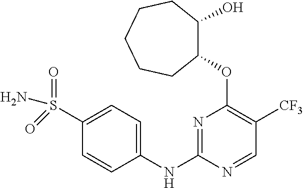 [SMILES: NS(=O)(=O)c1ccc(Nc2ncc(C(F)(F)F)c(O[C@@H]3CCCCC[C@@H]3O)n2)cc1]

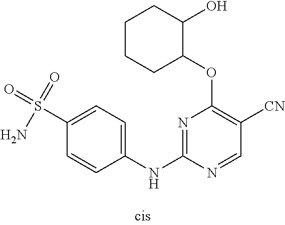 [SMILES: N#Cc1cnc(Nc2ccc(S(N)(=O)=O)cc2)nc1OC1CCCCC1O]

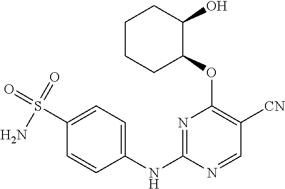 [SMILES: N#Cc1cnc(Nc2ccc(S(N)(=O)=O)cc2)nc1O[C@H]1CCCC[C@H]1O]

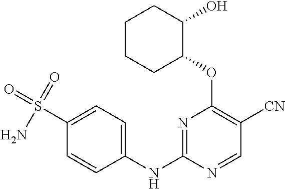 [SMILES: N#Cc1cnc(Nc2ccc(S(N)(=O)=O)cc2)nc1O[C@@H]1CCCC[C@@H]1O]

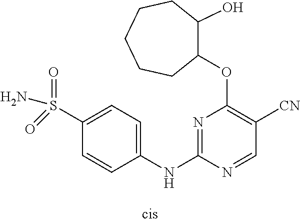 [SMILES: N#Cc1cnc(Nc2ccc(S(N)(=O)=O)cc2)nc1OC1CCCCCC1O]

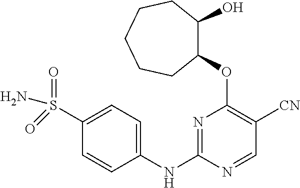 [SMILES: N#Cc1cnc(Nc2ccc(S(N)(=O)=O)cc2)nc1O[C@H]1CCCCC[C@H]1O]

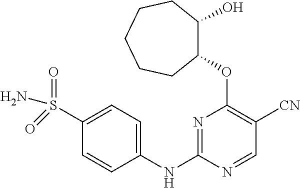 [SMILES: N#Cc1cnc(Nc2ccc(S(N)(=O)=O)cc2)nc1O[C@@H]1CCCCC[C@@H]1O]

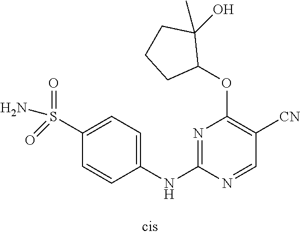 [SMILES: CC1(O)CCCC1Oc1nc(Nc2ccc(S(N)(=O)=O)cc2)ncc1C#N]

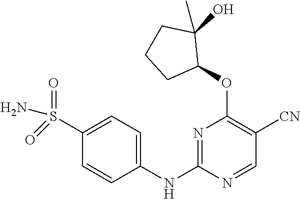 [SMILES: C[C@@]1(O)CCC[C@@H]1Oc1nc(Nc2ccc(S(N)(=O)=O)cc2)ncc1C#N]

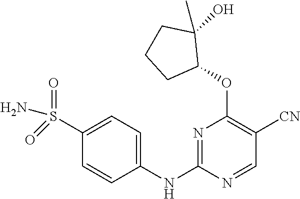 [SMILES: C[C@]1(O)CCC[C@H]1Oc1nc(Nc2ccc(S(N)(=O)=O)cc2)ncc1C#N]

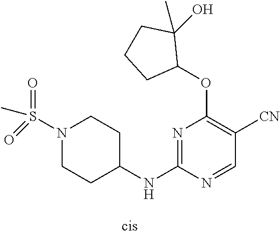 [SMILES: CC1(O)CCCC1Oc1nc(NC2CCN(S(C)(=O)=O)CC2)ncc1C#N]

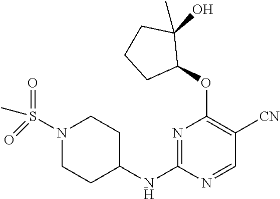 [SMILES: C[C@@]1(O)CCC[C@@H]1Oc1nc(NC2CCN(S(C)(=O)=O)CC2)ncc1C#N]

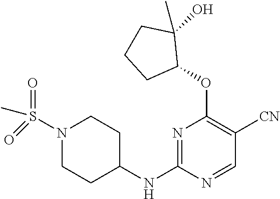 [SMILES: C[C@]1(O)CCC[C@H]1Oc1nc(NC2CCN(S(C)(=O)=O)CC2)ncc1C#N]

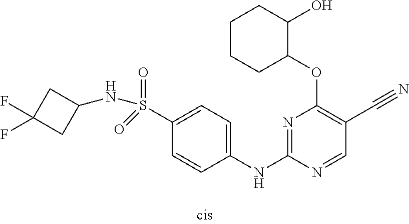 [SMILES: N#Cc1cnc(Nc2ccc(S(=O)(=O)NC3CC(F)(F)C3)cc2)nc1OC1CCCCC1O]

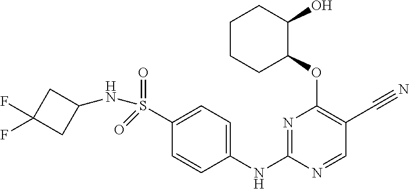 [SMILES: N#Cc1cnc(Nc2ccc(S(=O)(=O)NC3CC(F)(F)C3)cc2)nc1O[C@H]1CCCC[C@H]1O]

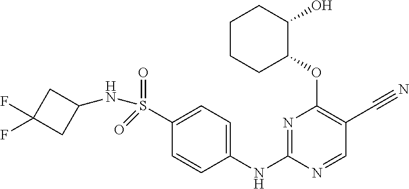 [SMILES: N#Cc1cnc(Nc2ccc(S(=O)(=O)NC3CC(F)(F)C3)cc2)nc1O[C@@H]1CCCC[C@@H]1O]

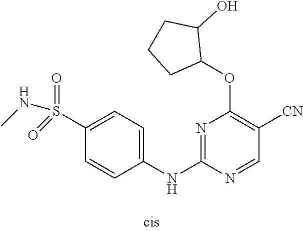 [SMILES: CNS(=O)(=O)c1ccc(Nc2ncc(C#N)c(OC3CCCC3O)n2)cc1]

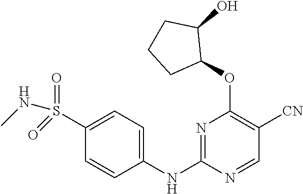 [SMILES: CNS(=O)(=O)c1ccc(Nc2ncc(C#N)c(O[C@H]3CCC[C@H]3O)n2)cc1]

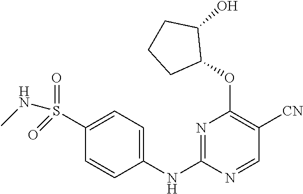 [SMILES: CNS(=O)(=O)c1ccc(Nc2ncc(C#N)c(O[C@@H]3CCC[C@@H]3O)n2)cc1]

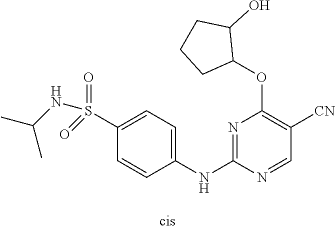 [SMILES: CC(C)NS(=O)(=O)c1ccc(Nc2ncc(C#N)c(OC3CCCC3O)n2)cc1]

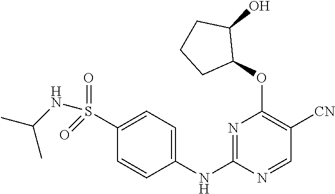 [SMILES: CC(C)NS(=O)(=O)c1ccc(Nc2ncc(C#N)c(O[C@H]3CCC[C@H]3O)n2)cc1]

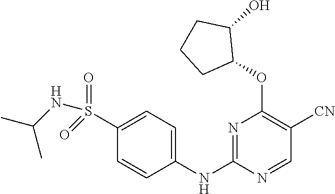 [SMILES: CC(C)NS(=O)(=O)c1ccc(Nc2ncc(C#N)c(O[C@@H]3CCC[C@@H]3O)n2)cc1]

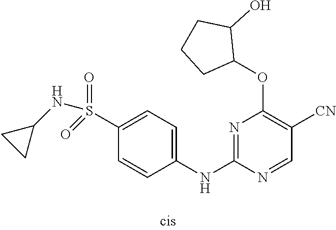 [SMILES: N#Cc1cnc(Nc2ccc(S(=O)(=O)NC3CC3)cc2)nc1OC1CCCC1O]

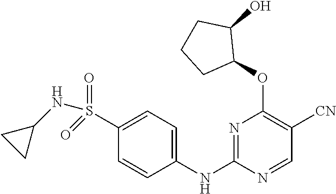 [SMILES: N#Cc1cnc(Nc2ccc(S(=O)(=O)NC3CC3)cc2)nc1O[C@H]1CCC[C@H]1O]

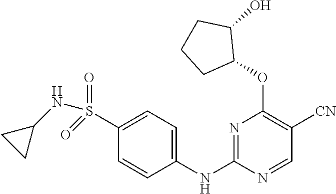 [SMILES: N#Cc1cnc(Nc2ccc(S(=O)(=O)NC3CC3)cc2)nc1O[C@@H]1CCC[C@@H]1O]

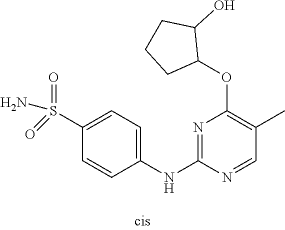 [SMILES: Cc1cnc(Nc2ccc(S(N)(=O)=O)cc2)nc1OC1CCCC1O]

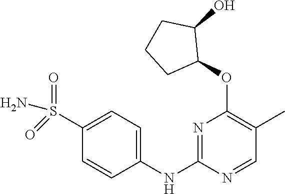 [SMILES: Cc1cnc(Nc2ccc(S(N)(=O)=O)cc2)nc1O[C@H]1CCC[C@H]1O]

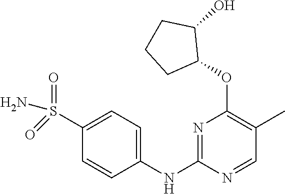 [SMILES: Cc1cnc(Nc2ccc(S(N)(=O)=O)cc2)nc1O[C@@H]1CCC[C@@H]1O]

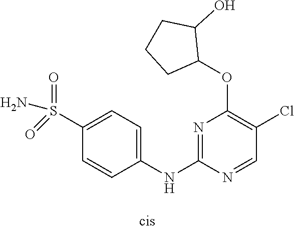 [SMILES: NS(=O)(=O)c1ccc(Nc2ncc(Cl)c(OC3CCCC3O)n2)cc1]

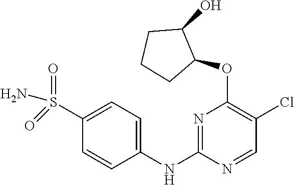 [SMILES: NS(=O)(=O)c1ccc(Nc2ncc(Cl)c(O[C@H]3CCC[C@H]3O)n2)cc1]

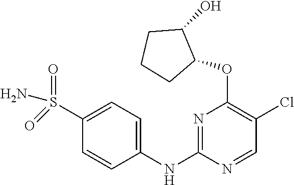 [SMILES: NS(=O)(=O)c1ccc(Nc2ncc(Cl)c(O[C@@H]3CCC[C@@H]3O)n2)cc1]

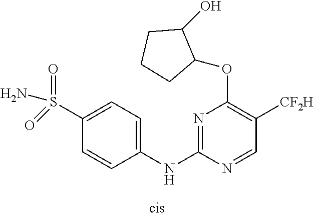 [SMILES: NS(=O)(=O)c1ccc(Nc2ncc(C(F)F)c(OC3CCCC3O)n2)cc1]

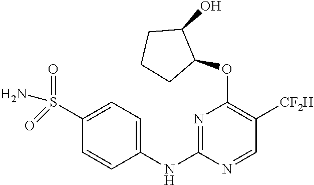 [SMILES: NS(=O)(=O)c1ccc(Nc2ncc(C(F)F)c(O[C@H]3CCC[C@H]3O)n2)cc1]

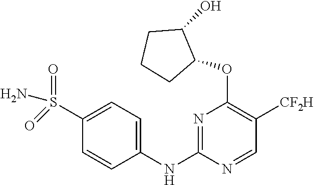 [SMILES: NS(=O)(=O)c1ccc(Nc2ncc(C(F)F)c(O[C@@H]3CCC[C@@H]3O)n2)cc1]

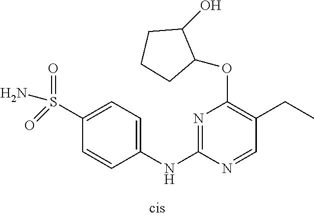 [SMILES: CCc1cnc(Nc2ccc(S(N)(=O)=O)cc2)nc1OC1CCCC1O]

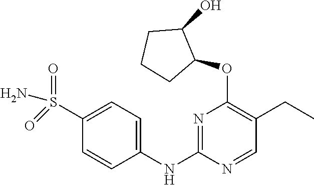 [SMILES: CCc1cnc(Nc2ccc(S(N)(=O)=O)cc2)nc1O[C@H]1CCC[C@H]1O]

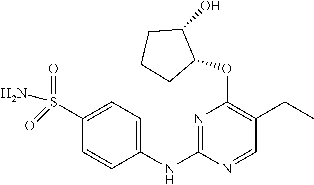 [SMILES: CCc1cnc(Nc2ccc(S(N)(=O)=O)cc2)nc1O[C@@H]1CCC[C@@H]1O]

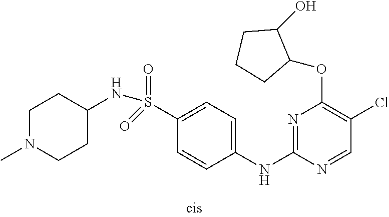 [SMILES: CN1CCC(NS(=O)(=O)c2ccc(Nc3ncc(Cl)c(OC4CCCC4O)n3)cc2)CC1]

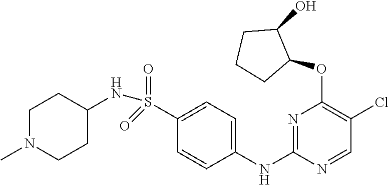 [SMILES: CN1CCC(NS(=O)(=O)c2ccc(Nc3ncc(Cl)c(O[C@H]4CCC[C@H]4O)n3)cc2)CC1]

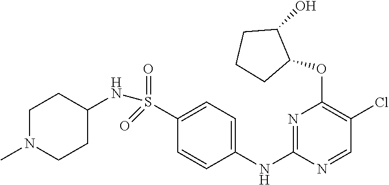 [SMILES: CN1CCC(NS(=O)(=O)c2ccc(Nc3ncc(Cl)c(O[C@@H]4CCC[C@@H]4O)n3)cc2)CC1]

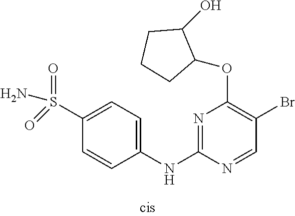 [SMILES: NS(=O)(=O)c1ccc(Nc2ncc(Br)c(OC3CCCC3O)n2)cc1]

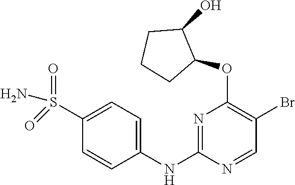 [SMILES: NS(=O)(=O)c1ccc(Nc2ncc(Br)c(O[C@H]3CCC[C@H]3O)n2)cc1]

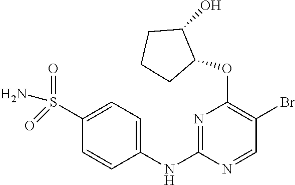 [SMILES: NS(=O)(=O)c1ccc(Nc2ncc(Br)c(O[C@@H]3CCC[C@@H]3O)n2)cc1]

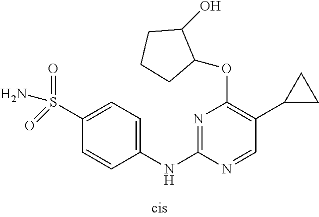 [SMILES: NS(=O)(=O)c1ccc(Nc2ncc(C3CC3)c(OC3CCCC3O)n2)cc1]

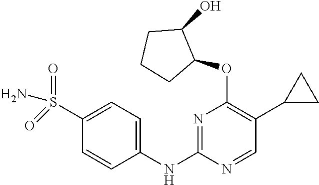 [SMILES: NS(=O)(=O)c1ccc(Nc2ncc(C3CC3)c(O[C@H]3CCC[C@H]3O)n2)cc1]

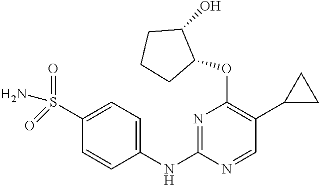 [SMILES: NS(=O)(=O)c1ccc(Nc2ncc(C3CC3)c(O[C@@H]3CCC[C@@H]3O)n2)cc1]

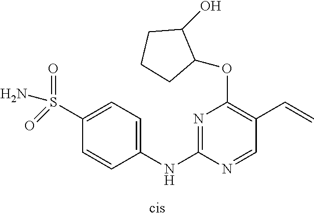 [SMILES: C=Cc1cnc(Nc2ccc(S(N)(=O)=O)cc2)nc1OC1CCCC1O]

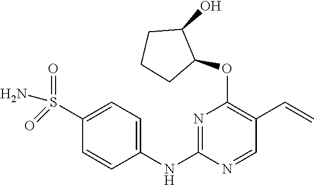 [SMILES: C=Cc1cnc(Nc2ccc(S(N)(=O)=O)cc2)nc1O[C@H]1CCC[C@H]1O]

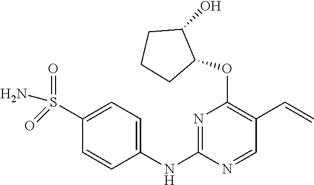 [SMILES: C=Cc1cnc(Nc2ccc(S(N)(=O)=O)cc2)nc1O[C@@H]1CCC[C@@H]1O]

TABLE 1B
List of Compounds

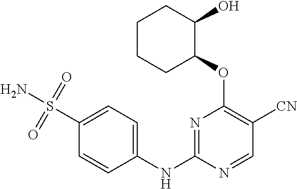 [SMILES: N#Cc1cnc(Nc2ccc(S(N)(=O)=O)cc2)nc1O[C@H]1CCCC[C@H]1O]

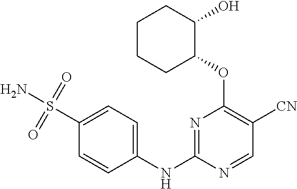 [SMILES: N#Cc1cnc(Nc2ccc(S(N)(=O)=O)cc2)nc1O[C@@H]1CCCC[C@@H]1O]

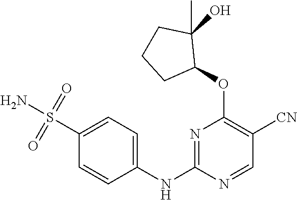 [SMILES: C[C@@]1(O)CCC[C@@H]1Oc1nc(Nc2ccc(S(N)(=O)=O)cc2)ncc1C#N]

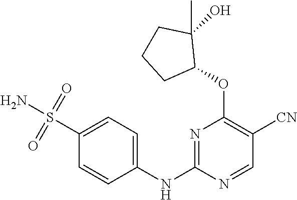 [SMILES: C[C@]1(O)CCC[C@H]1Oc1nc(Nc2ccc(S(N)(=O)=O)cc2)ncc1C#N]

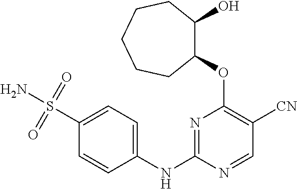 [SMILES: N#Cc1cnc(Nc2ccc(S(N)(=O)=O)cc2)nc1O[C@H]1CCCCC[C@H]1O]

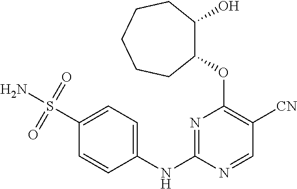 [SMILES: N#Cc1cnc(Nc2ccc(S(N)(=O)=O)cc2)nc1O[C@@H]1CCCCC[C@@H]1O]

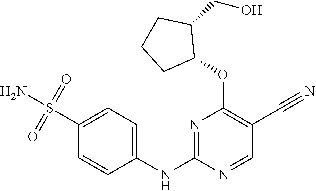 [SMILES: N#Cc1cnc(Nc2ccc(S(N)(=O)=O)cc2)nc1O[C@@H]1CCC[C@@H]1CO]

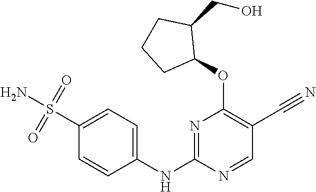 [SMILES: N#Cc1cnc(Nc2ccc(S(N)(=O)=O)cc2)nc1O[C@H]1CCC[C@H]1CO]

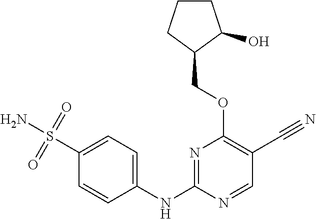 [SMILES: N#Cc1cnc(Nc2ccc(S(N)(=O)=O)cc2)nc1OC[C@H]1CCC[C@H]1O]

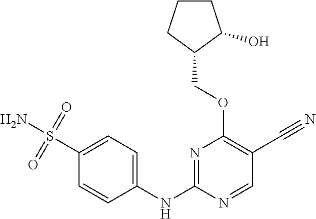 [SMILES: N#Cc1cnc(Nc2ccc(S(N)(=O)=O)cc2)nc1OC[C@@H]1CCC[C@@H]1O]

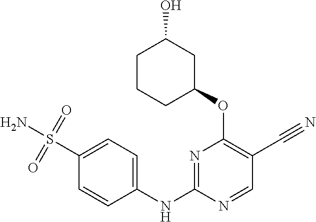 [SMILES: N#Cc1cnc(Nc2ccc(S(N)(=O)=O)cc2)nc1O[C@H]1CCC[C@H](O)C1]

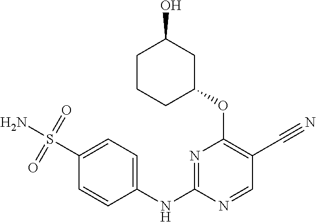 [SMILES: N#Cc1cnc(Nc2ccc(S(N)(=O)=O)cc2)nc1O[C@@H]1CCC[C@@H](O)C1]

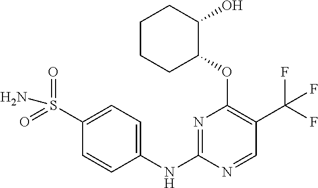 [SMILES: NS(=O)(=O)c1ccc(Nc2ncc(C(F)(F)F)c(O[C@@H]3CCCC[C@@H]3O)n2)cc1]

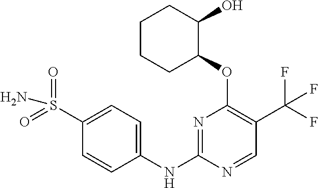 [SMILES: NS(=O)(=O)c1ccc(Nc2ncc(C(F)(F)F)c(O[C@H]3CCCC[C@H]3O)n2)cc1]

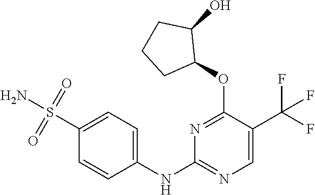 [SMILES: NS(=O)(=O)c1ccc(Nc2ncc(C(F)(F)F)c(O[C@H]3CCC[C@H]3O)n2)cc1]

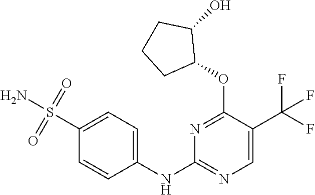 [SMILES: NS(=O)(=O)c1ccc(Nc2ncc(C(F)(F)F)c(O[C@@H]3CCC[C@@H]3O)n2)cc1]

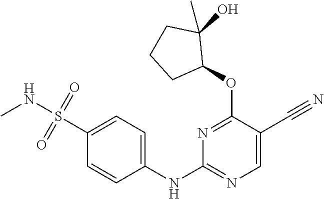 [SMILES: CNS(=O)(=O)c1ccc(Nc2ncc(C#N)c(O[C@H]3CCC[C@@]3(C)O)n2)cc1]

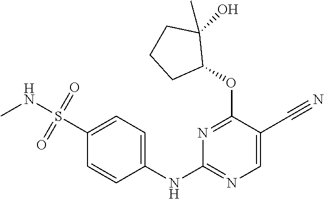 [SMILES: CNS(=O)(=O)c1ccc(Nc2ncc(C#N)c(O[C@@H]3CCC[C@]3(C)O)n2)cc1]

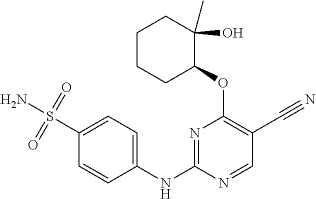 [SMILES: C[C@@]1(O)CCCC[C@@H]1Oc1nc(Nc2ccc(S(N)(=O)=O)cc2)ncc1C#N]

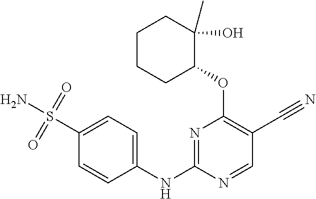 [SMILES: C[C@]1(O)CCCC[C@H]1Oc1nc(Nc2ccc(S(N)(=O)=O)cc2)ncc1C#N]

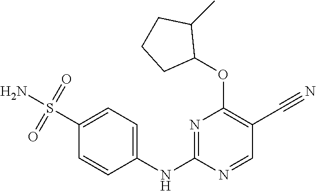 [SMILES: CC1CCCC1Oc1nc(Nc2ccc(S(N)(=O)=O)cc2)ncc1C#N]

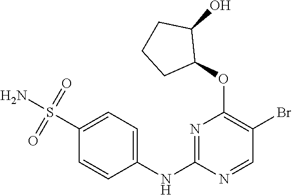 [SMILES: NS(=O)(=O)c1ccc(Nc2ncc(Br)c(O[C@H]3CCC[C@H]3O)n2)cc1]

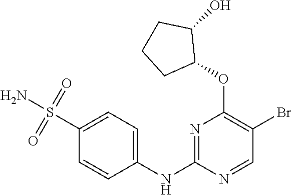 [SMILES: NS(=O)(=O)c1ccc(Nc2ncc(Br)c(O[C@@H]3CCC[C@@H]3O)n2)cc1]

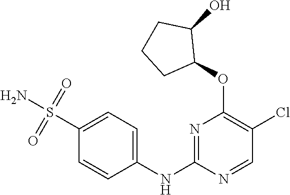 [SMILES: NS(=O)(=O)c1ccc(Nc2ncc(Cl)c(O[C@H]3CCC[C@H]3O)n2)cc1]

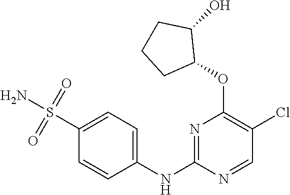 [SMILES: NS(=O)(=O)c1ccc(Nc2ncc(Cl)c(O[C@@H]3CCC[C@@H]3O)n2)cc1]

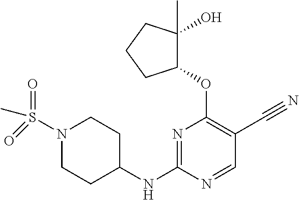 [SMILES: C[C@]1(O)CCC[C@H]1Oc1nc(NC2CCN(S(C)(=O)=O)CC2)ncc1C#N]

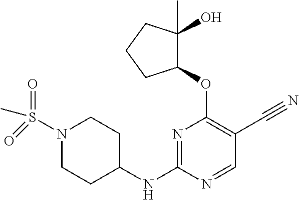 [SMILES: C[C@@]1(O)CCC[C@@H]1Oc1nc(NC2CCN(S(C)(=O)=O)CC2)ncc1C#N]

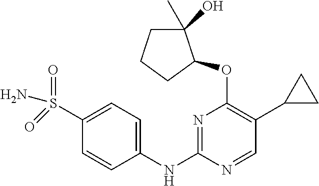 [SMILES: C[C@@]1(O)CCC[C@@H]1Oc1nc(Nc2ccc(S(N)(=O)=O)cc2)ncc1C1CC1]

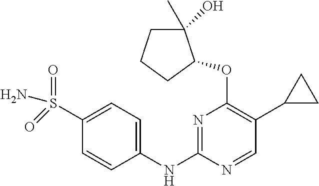 [SMILES: C[C@]1(O)CCC[C@H]1Oc1nc(Nc2ccc(S(N)(=O)=O)cc2)ncc1C1CC1]

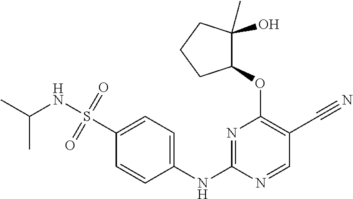 [SMILES: CC(C)NS(=O)(=O)c1ccc(Nc2ncc(C#N)c(O[C@H]3CCC[C@@]3(C)O)n2)cc1]

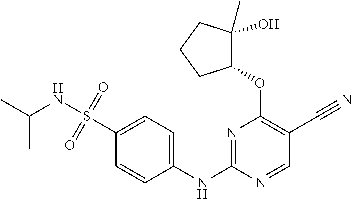 [SMILES: CC(C)NS(=O)(=O)c1ccc(Nc2ncc(C#N)c(O[C@@H]3CCC[C@]3(C)O)n2)cc1]

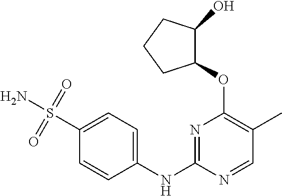 [SMILES: Cc1cnc(Nc2ccc(S(N)(=O)=O)cc2)nc1O[C@H]1CCC[C@H]1O]

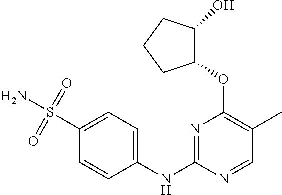 [SMILES: Cc1cnc(Nc2ccc(S(N)(=O)=O)cc2)nc1O[C@@H]1CCC[C@@H]1O]

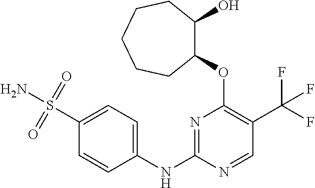 [SMILES: NS(=O)(=O)c1ccc(Nc2ncc(C(F)(F)F)c(O[C@H]3CCCCC[C@H]3O)n2)cc1]

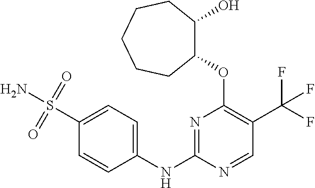 [SMILES: NS(=O)(=O)c1ccc(Nc2ncc(C(F)(F)F)c(O[C@@H]3CCCCC[C@@H]3O)n2)cc1]

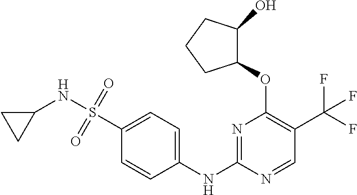 [SMILES: O=S(=O)(NC1CC1)c1ccc(Nc2ncc(C(F)(F)F)c(O[C@H]3CCC[C@H]3O)n2)cc1]

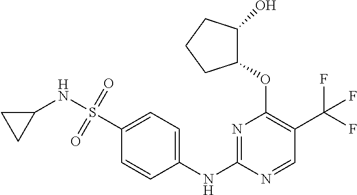 [SMILES: O=S(=O)(NC1CC1)c1ccc(Nc2ncc(C(F)(F)F)c(O[C@@H]3CCC[C@@H]3O)n2)cc1]

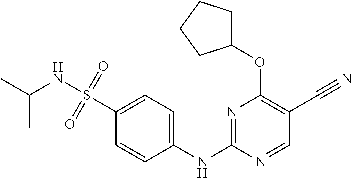 [SMILES: CC(C)NS(=O)(=O)c1ccc(Nc2ncc(C#N)c(OC3CCCC3)n2)cc1]

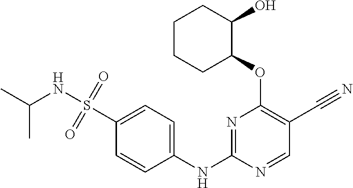 [SMILES: CC(C)NS(=O)(=O)c1ccc(Nc2ncc(C#N)c(O[C@H]3CCCC[C@H]3O)n2)cc1]

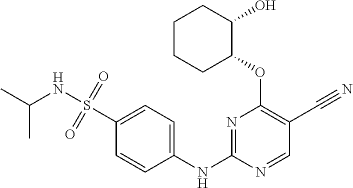 [SMILES: CC(C)NS(=O)(=O)c1ccc(Nc2ncc(C#N)c(O[C@@H]3CCCC[C@@H]3O)n2)cc1]

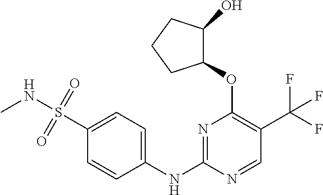 [SMILES: CNS(=O)(=O)c1ccc(Nc2ncc(C(F)(F)F)c(O[C@H]3CCC[C@H]3O)n2)cc1]

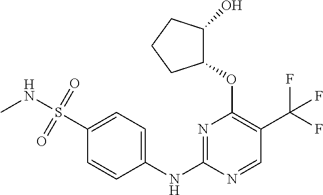 [SMILES: CNS(=O)(=O)c1ccc(Nc2ncc(C(F)(F)F)c(O[C@@H]3CCC[C@@H]3O)n2)cc1]

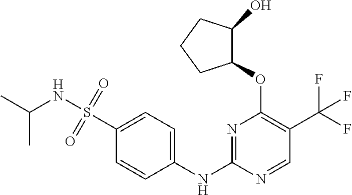 [SMILES: CC(C)NS(=O)(=O)c1ccc(Nc2ncc(C(F)(F)F)c(O[C@H]3CCC[C@H]3O)n2)cc1]

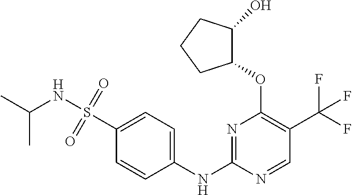 [SMILES: CC(C)NS(=O)(=O)c1ccc(Nc2ncc(C(F)(F)F)c(O[C@@H]3CCC[C@@H]3O)n2)cc1]

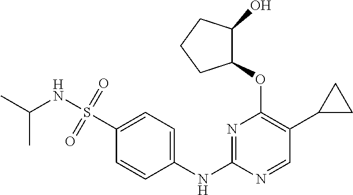 [SMILES: CC(C)NS(=O)(=O)c1ccc(Nc2ncc(C3CC3)c(O[C@H]3CCC[C@H]3O)n2)cc1]

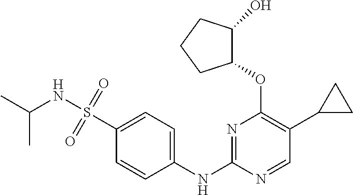 [SMILES: CC(C)NS(=O)(=O)c1ccc(Nc2ncc(C3CC3)c(O[C@@H]3CCC[C@@H]3O)n2)cc1]

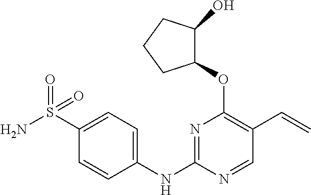 [SMILES: C=Cc1cnc(Nc2ccc(S(N)(=O)=O)cc2)nc1O[C@H]1CCC[C@H]1O]

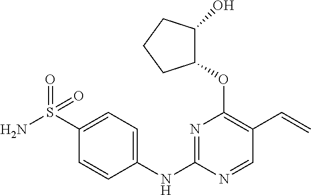 [SMILES: C=Cc1cnc(Nc2ccc(S(N)(=O)=O)cc2)nc1O[C@@H]1CCC[C@@H]1O]

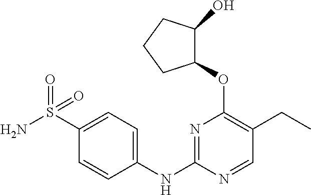 [SMILES: CCc1cnc(Nc2ccc(S(N)(=O)=O)cc2)nc1O[C@H]1CCC[C@H]1O]

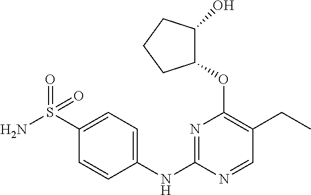 [SMILES: CCc1cnc(Nc2ccc(S(N)(=O)=O)cc2)nc1O[C@@H]1CCC[C@@H]1O]

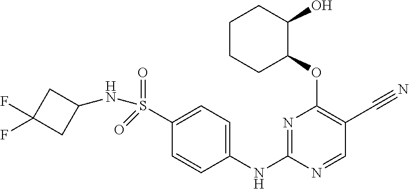 [SMILES: N#Cc1cnc(Nc2ccc(S(=O)(=O)NC3CC(F)(F)C3)cc2)nc1O[C@H]1CCCC[C@H]1O]

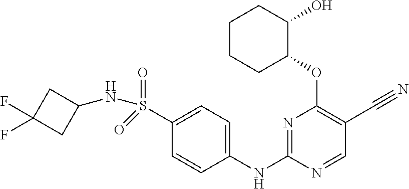 [SMILES: N#Cc1cnc(Nc2ccc(S(=O)(=O)NC3CC(F)(F)C3)cc2)nc1O[C@@H]1CCCC[C@@H]1O]

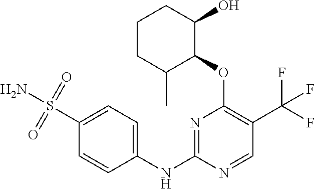 [SMILES: CC1CCC[C@@H](O)[C@H]1Oc1nc(Nc2ccc(S(N)(=O)=O)cc2)ncc1C(F)(F)F]

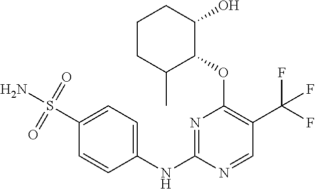 [SMILES: CC1CCC[C@H](O)[C@@H]1Oc1nc(Nc2ccc(S(N)(=O)=O)cc2)ncc1C(F)(F)F]

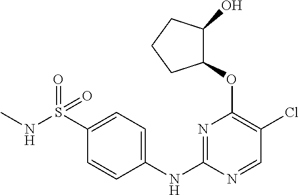 [SMILES: CNS(=O)(=O)c1ccc(Nc2ncc(Cl)c(O[C@H]3CCC[C@H]3O)n2)cc1]

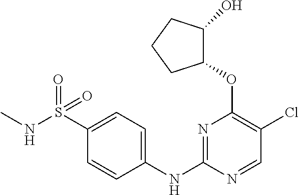 [SMILES: CNS(=O)(=O)c1ccc(Nc2ncc(Cl)c(O[C@@H]3CCC[C@@H]3O)n2)cc1]

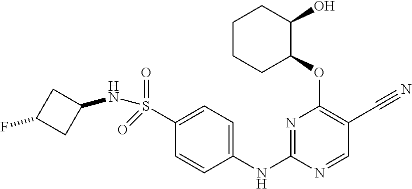

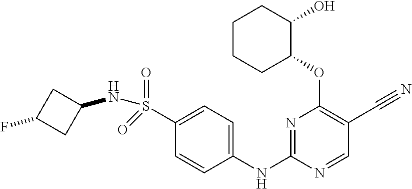

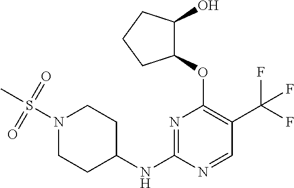 [SMILES: CS(=O)(=O)N1CCC(Nc2ncc(C(F)(F)F)c(O[C@H]3CCC[C@H]3O)n2)CC1]

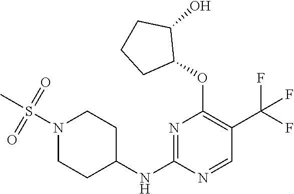 [SMILES: CS(=O)(=O)N1CCC(Nc2ncc(C(F)(F)F)c(O[C@@H]3CCC[C@@H]3O)n2)CC1]

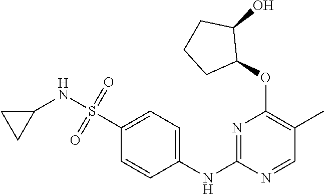 [SMILES: Cc1cnc(Nc2ccc(S(=O)(=O)NC3CC3)cc2)nc1O[C@H]1CCC[C@H]1O]

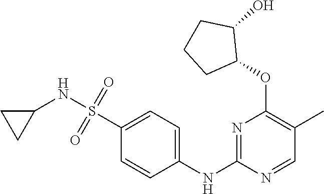 [SMILES: Cc1cnc(Nc2ccc(S(=O)(=O)NC3CC3)cc2)nc1O[C@@H]1CCC[C@@H]1O]

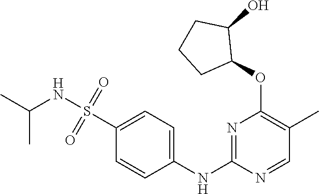 [SMILES: Cc1cnc(Nc2ccc(S(=O)(=O)NC(C)C)cc2)nc1O[C@H]1CCC[C@H]1O]

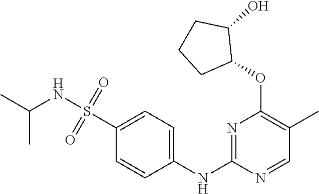 [SMILES: Cc1cnc(Nc2ccc(S(=O)(=O)NC(C)C)cc2)nc1O[C@@H]1CCC[C@@H]1O]

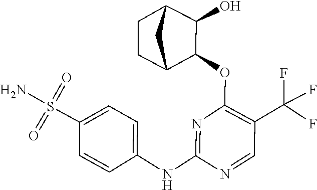 [SMILES: NS(=O)(=O)c1ccc(Nc2ncc(C(F)(F)F)c(O[C@H]3[C@@H]4CC[C@@H](C4)[C@H]3O)n2)cc1]

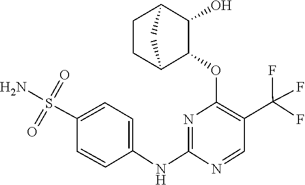 [SMILES: NS(=O)(=O)c1ccc(Nc2ncc(C(F)(F)F)c(O[C@@H]3[C@H]4CC[C@H](C4)[C@@H]3O)n2)cc1]

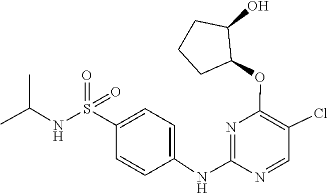 [SMILES: CC(C)NS(=O)(=O)c1ccc(Nc2ncc(Cl)c(O[C@H]3CCC[C@H]3O)n2)cc1]

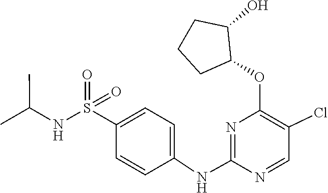 [SMILES: CC(C)NS(=O)(=O)c1ccc(Nc2ncc(Cl)c(O[C@@H]3CCC[C@@H]3O)n2)cc1]

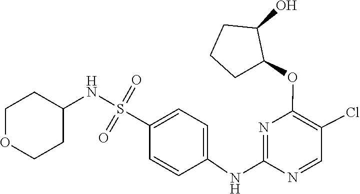 [SMILES: O=S(=O)(NC1CCOCC1)c1ccc(Nc2ncc(Cl)c(O[C@H]3CCC[C@H]3O)n2)cc1]

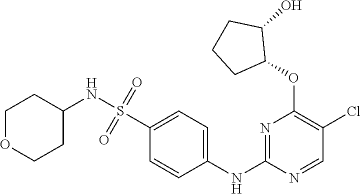 [SMILES: O=S(=O)(NC1CCOCC1)c1ccc(Nc2ncc(Cl)c(O[C@@H]3CCC[C@@H]3O)n2)cc1]

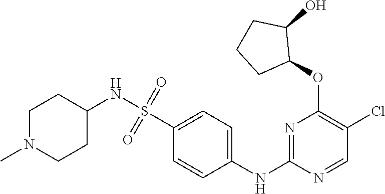 [SMILES: CN1CCC(NS(=O)(=O)c2ccc(Nc3ncc(Cl)c(O[C@H]4CCC[C@H]4O)n3)cc2)CC1]

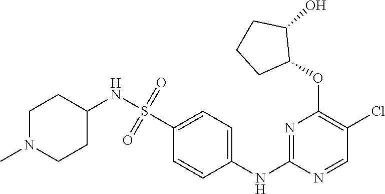 [SMILES: CN1CCC(NS(=O)(=O)c2ccc(Nc3ncc(Cl)c(O[C@@H]4CCC[C@@H]4O)n3)cc2)CC1]

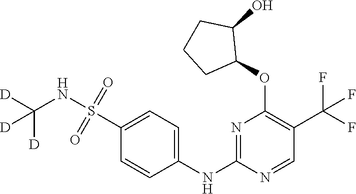 [SMILES: [2H]C([2H])([2H])NS(=O)(=O)c1ccc(Nc2ncc(C(F)(F)F)c(O[C@H]3CCC[C@H]3O)n2)cc1]

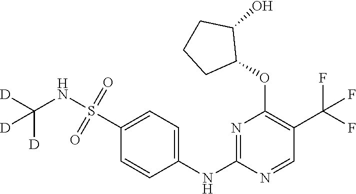 [SMILES: [2H]C([2H])([2H])NS(=O)(=O)c1ccc(Nc2ncc(C(F)(F)F)c(O[C@@H]3CCC[C@@H]3O)n2)cc1]

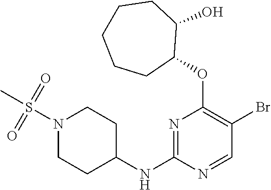 [SMILES: CS(=O)(=O)N1CCC(Nc2ncc(Br)c(O[C@@H]3CCCCC[C@@H]3O)n2)CC1]

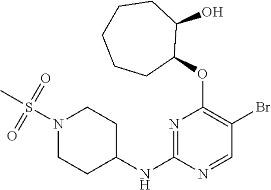 [SMILES: CS(=O)(=O)N1CCC(Nc2ncc(Br)c(O[C@H]3CCCCC[C@H]3O)n2)CC1]

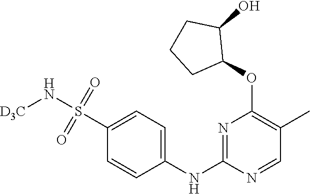 [SMILES: [2H]C([2H])([2H])NS(=O)(=O)c1ccc(Nc2ncc(C)c(O[C@H]3CCC[C@H]3O)n2)cc1]

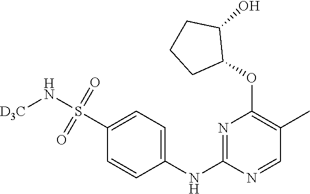 [SMILES: [2H]C([2H])([2H])NS(=O)(=O)c1ccc(Nc2ncc(C)c(O[C@@H]3CCC[C@@H]3O)n2)cc1]

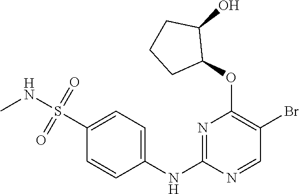 [SMILES: CNS(=O)(=O)c1ccc(Nc2ncc(Br)c(O[C@H]3CCC[C@H]3O)n2)cc1]

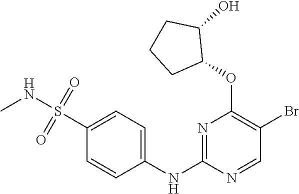 [SMILES: CNS(=O)(=O)c1ccc(Nc2ncc(Br)c(O[C@@H]3CCC[C@@H]3O)n2)cc1]

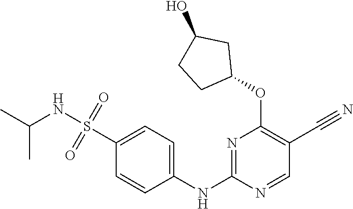 [SMILES: CC(C)NS(=O)(=O)c1ccc(Nc2ncc(C#N)c(O[C@@H]3CC[C@@H](O)C3)n2)cc1]

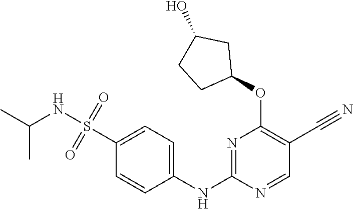 [SMILES: CC(C)NS(=O)(=O)c1ccc(Nc2ncc(C#N)c(O[C@H]3CC[C@H](O)C3)n2)cc1]

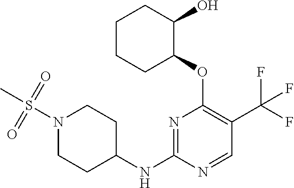 [SMILES: CS(=O)(=O)N1CCC(Nc2ncc(C(F)(F)F)c(O[C@H]3CCCC[C@H]3O)n2)CC1]

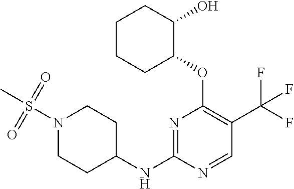 [SMILES: CS(=O)(=O)N1CCC(Nc2ncc(C(F)(F)F)c(O[C@@H]3CCCC[C@@H]3O)n2)CC1]

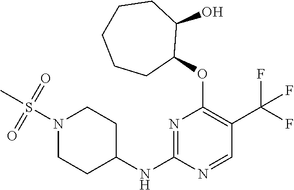 [SMILES: CS(=O)(=O)N1CCC(Nc2ncc(C(F)(F)F)c(O[C@H]3CCCCC[C@H]3O)n2)CC1]

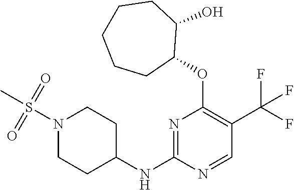 [SMILES: CS(=O)(=O)N1CCC(Nc2ncc(C(F)(F)F)c(O[C@@H]3CCCCC[C@@H]3O)n2)CC1]

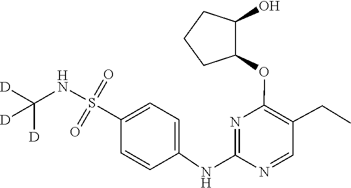 [SMILES: [2H]C([2H])([2H])NS(=O)(=O)c1ccc(Nc2ncc(CC)c(O[C@H]3CCC[C@H]3O)n2)cc1]

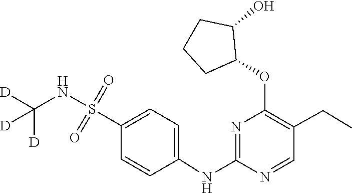 [SMILES: [2H]C([2H])([2H])NS(=O)(=O)c1ccc(Nc2ncc(CC)c(O[C@@H]3CCC[C@@H]3O)n2)cc1]

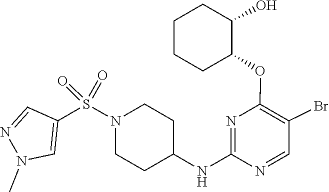 [SMILES: Cn1cc(S(=O)(=O)N2CCC(Nc3ncc(Br)c(O[C@@H]4CCCC[C@@H]4O)n3)CC2)cn1]

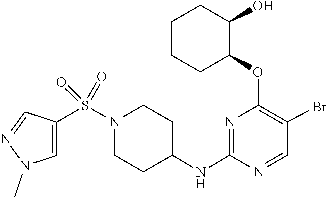 [SMILES: Cn1cc(S(=O)(=O)N2CCC(Nc3ncc(Br)c(O[C@H]4CCCC[C@H]4O)n3)CC2)cn1]

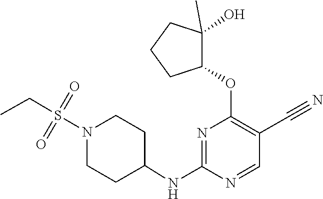 [SMILES: CCS(=O)(=O)N1CCC(Nc2ncc(C#N)c(O[C@@H]3CCC[C@]3(C)O)n2)CC1]

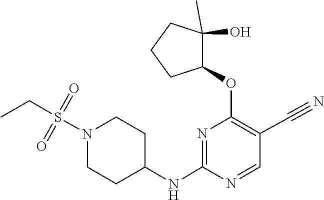 [SMILES: CCS(=O)(=O)N1CCC(Nc2ncc(C#N)c(O[C@H]3CCC[C@@]3(C)O)n2)CC1]

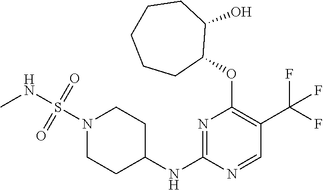 [SMILES: CNS(=O)(=O)N1CCC(Nc2ncc(C(F)(F)F)c(O[C@@H]3CCCCC[C@@H]3O)n2)CC1]

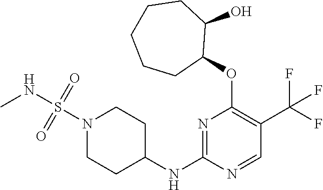 [SMILES: CNS(=O)(=O)N1CCC(Nc2ncc(C(F)(F)F)c(O[C@H]3CCCCC[C@H]3O)n2)CC1]

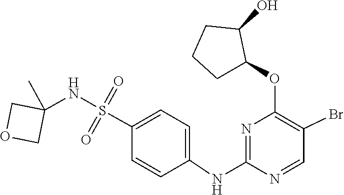 [SMILES: CC1(NS(=O)(=O)c2ccc(Nc3ncc(Br)c(O[C@H]4CCC[C@H]4O)n3)cc2)COC1]

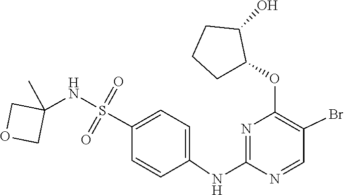 [SMILES: CC1(NS(=O)(=O)c2ccc(Nc3ncc(Br)c(O[C@@H]4CCC[C@@H]4O)n3)cc2)COC1]

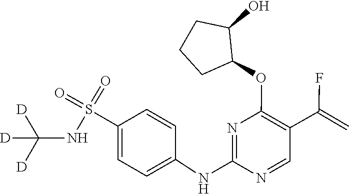 [SMILES: [2H]C([2H])([2H])NS(=O)(=O)c1ccc(Nc2ncc(C(=C)F)c(O[C@H]3CCC[C@H]3O)n2)cc1]

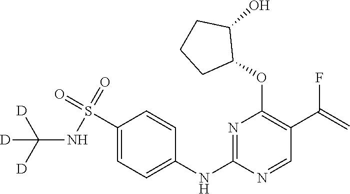 [SMILES: [2H]C([2H])([2H])NS(=O)(=O)c1ccc(Nc2ncc(C(=C)F)c(O[C@@H]3CCC[C@@H]3O)n2)cc1]

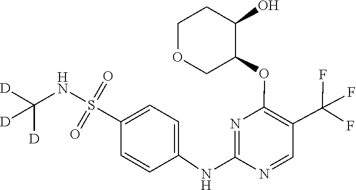 [SMILES: [2H]C([2H])([2H])NS(=O)(=O)c1ccc(Nc2ncc(C(F)(F)F)c(O[C@H]3COCC[C@H]3O)n2)cc1]

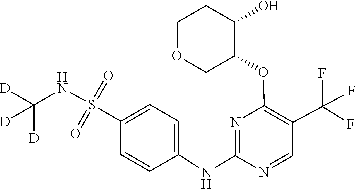 [SMILES: [2H]C([2H])([2H])NS(=O)(=O)c1ccc(Nc2ncc(C(F)(F)F)c(O[C@@H]3COCC[C@@H]3O)n2)cc1]

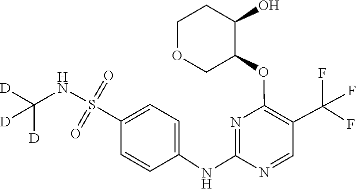 [SMILES: [2H]C([2H])([2H])NS(=O)(=O)c1ccc(Nc2ncc(C(F)(F)F)c(O[C@H]3COCC[C@H]3O)n2)cc1]

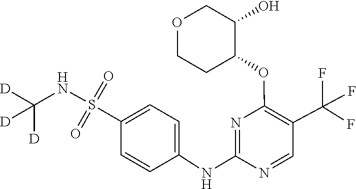 [SMILES: [2H]C([2H])([2H])NS(=O)(=O)c1ccc(Nc2ncc(C(F)(F)F)c(O[C@@H]3CCOC[C@@H]3O)n2)cc1]

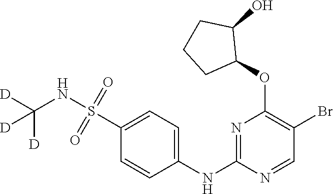 [SMILES: [2H]C([2H])([2H])NS(=O)(=O)c1ccc(Nc2ncc(Br)c(O[C@H]3CCC[C@H]3O)n2)cc1]

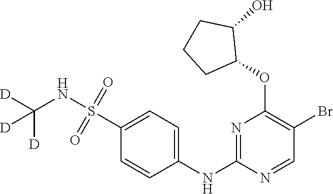 [SMILES: [2H]C([2H])([2H])NS(=O)(=O)c1ccc(Nc2ncc(Br)c(O[C@@H]3CCC[C@@H]3O)n2)cc1]

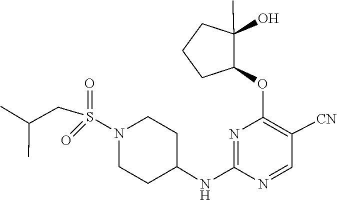 [SMILES: CC(C)CS(=O)(=O)N1CCC(Nc2ncc(C#N)c(O[C@H]3CCC[C@@]3(C)O)n2)CC1]

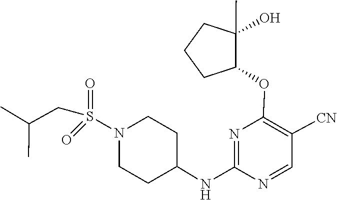 [SMILES: CC(C)CS(=O)(=O)N1CCC(Nc2ncc(C#N)c(O[C@@H]3CCC[C@]3(C)O)n2)CC1]

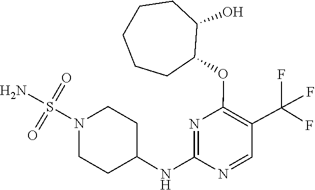 [SMILES: NS(=O)(=O)N1CCC(Nc2ncc(C(F)(F)F)c(O[C@@H]3CCCCC[C@@H]3O)n2)CC1]

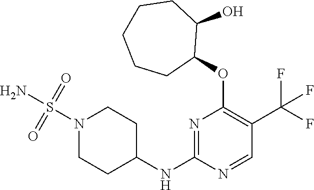 [SMILES: NS(=O)(=O)N1CCC(Nc2ncc(C(F)(F)F)c(O[C@H]3CCCCC[C@H]3O)n2)CC1]

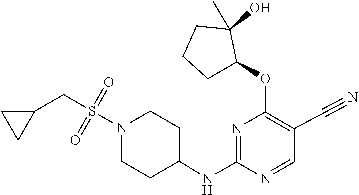 [SMILES: C[C@@]1(O)CCC[C@@H]1Oc1nc(NC2CCN(S(=O)(=O)CC3CC3)CC2)ncc1C#N]

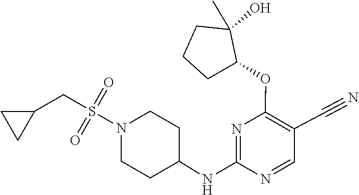 [SMILES: C[C@]1(O)CCC[C@H]1Oc1nc(NC2CCN(S(=O)(=O)CC3CC3)CC2)ncc1C#N]

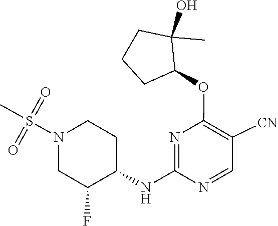 [SMILES: C[C@@]1(O)CCC[C@@H]1Oc1nc(N[C@H]2CCN(S(C)(=O)=O)C[C@H]2F)ncc1C#N]

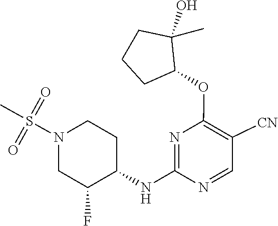 [SMILES: C[C@]1(O)CCC[C@H]1Oc1nc(N[C@H]2CCN(S(C)(=O)=O)C[C@H]2F)ncc1C#N]

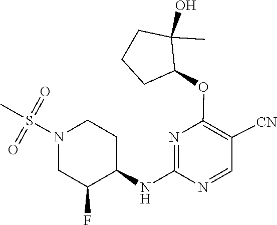 [SMILES: C[C@@]1(O)CCC[C@@H]1Oc1nc(N[C@@H]2CCN(S(C)(=O)=O)C[C@@H]2F)ncc1C#N]

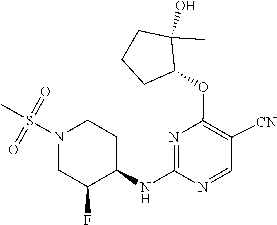 [SMILES: C[C@]1(O)CCC[C@H]1Oc1nc(N[C@@H]2CCN(S(C)(=O)=O)C[C@@H]2F)ncc1C#N]

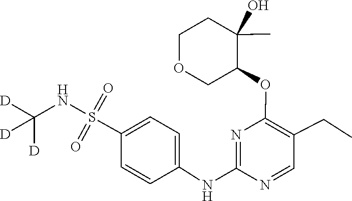 [SMILES: [2H]C([2H])([2H])NS(=O)(=O)c1ccc(Nc2ncc(CC)c(O[C@H]3COCC[C@@]3(C)O)n2)cc1]

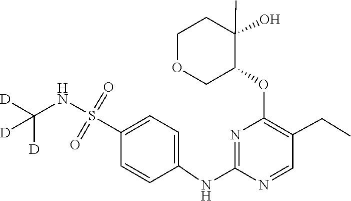 [SMILES: [2H]C([2H])([2H])NS(=O)(=O)c1ccc(Nc2ncc(CC)c(O[C@@H]3COCC[C@]3(C)O)n2)cc1]

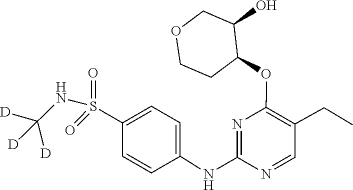 [SMILES: [2H]C([2H])([2H])NS(=O)(=O)c1ccc(Nc2ncc(CC)c(O[C@H]3CCOC[C@H]3O)n2)cc1]

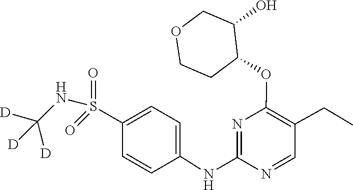 [SMILES: [2H]C([2H])([2H])NS(=O)(=O)c1ccc(Nc2ncc(CC)c(O[C@@H]3CCOC[C@@H]3O)n2)cc1]

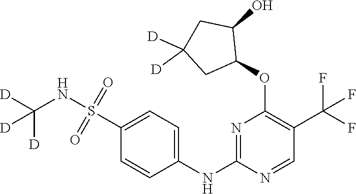 [SMILES: [2H]C1([2H])C[C@@H](O)[C@@H](Oc2nc(Nc3ccc(S(=O)(=O)NC([2H])([2H])[2H])cc3)ncc2C(F)(F)F)C1]

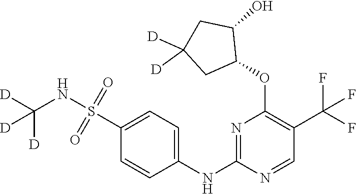 [SMILES: [2H]C1([2H])C[C@H](O)[C@H](Oc2nc(Nc3ccc(S(=O)(=O)NC([2H])([2H])[2H])cc3)ncc2C(F)(F)F)C1]

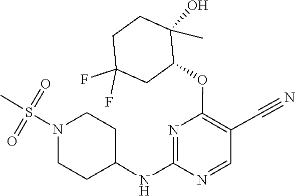 [SMILES: C[C@]1(O)CCC(F)(F)C[C@H]1Oc1nc(NC2CCN(S(C)(=O)=O)CC2)ncc1C#N]

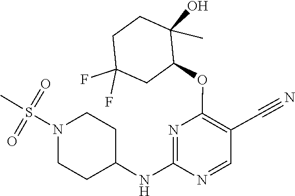 [SMILES: C[C@@]1(O)CCC(F)(F)C[C@@H]1Oc1nc(NC2CCN(S(C)(=O)=O)CC2)ncc1C#N]

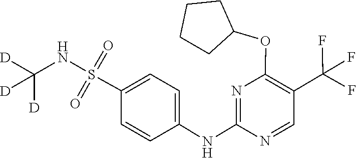 [SMILES: [2H]C([2H])([2H])NS(=O)(=O)c1ccc(Nc2ncc(C(F)(F)F)c(OC3CCCC3)n2)cc1]

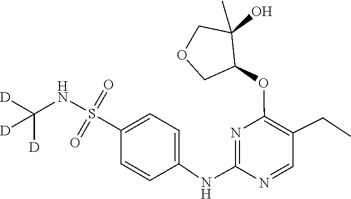 [SMILES: [2H]C([2H])([2H])NS(=O)(=O)c1ccc(Nc2ncc(CC)c(O[C@H]3COC[C@@]3(C)O)n2)cc1]

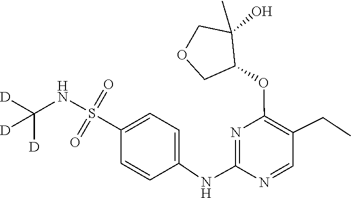 [SMILES: [2H]C([2H])([2H])NS(=O)(=O)c1ccc(Nc2ncc(CC)c(O[C@@H]3COC[C@]3(C)O)n2)cc1]

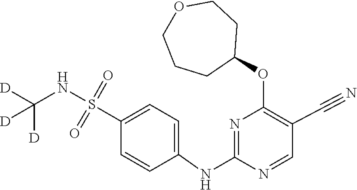 [SMILES: [2H]C([2H])([2H])NS(=O)(=O)c1ccc(Nc2ncc(C#N)c(O[C@H]3CCCOCC3)n2)cc1]

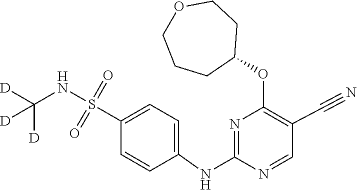 [SMILES: [2H]C([2H])([2H])NS(=O)(=O)c1ccc(Nc2ncc(C#N)c(O[C@@H]3CCCOCC3)n2)cc1]

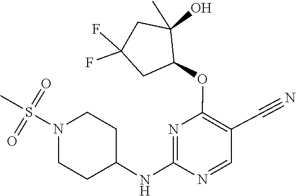 [SMILES: C[C@@]1(O)CC(F)(F)C[C@@H]1Oc1nc(NC2CCN(S(C)(=O)=O)CC2)ncc1C#N]

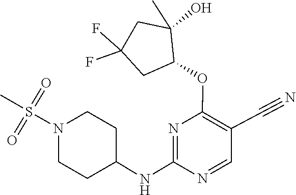 [SMILES: C[C@]1(O)CC(F)(F)C[C@H]1Oc1nc(NC2CCN(S(C)(=O)=O)CC2)ncc1C#N]

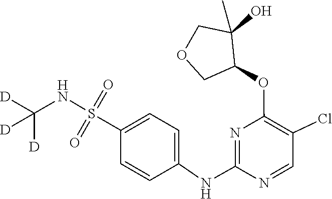 [SMILES: [2H]C([2H])([2H])NS(=O)(=O)c1ccc(Nc2ncc(Cl)c(O[C@H]3COC[C@@]3(C)O)n2)cc1]

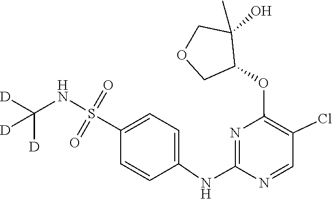 [SMILES: [2H]C([2H])([2H])NS(=O)(=O)c1ccc(Nc2ncc(Cl)c(O[C@@H]3COC[C@]3(C)O)n2)cc1]

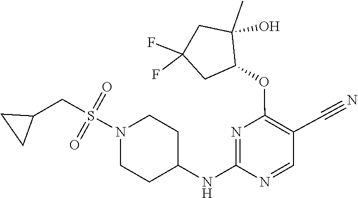 [SMILES: C[C@]1(O)CC(F)(F)C[C@H]1Oc1nc(NC2CCN(S(=O)(=O)CC3CC3)CC2)ncc1C#N]

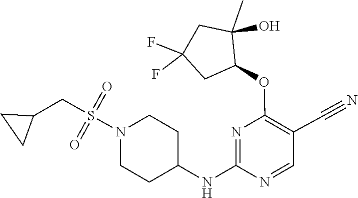 [SMILES: C[C@@]1(O)CC(F)(F)C[C@@H]1Oc1nc(NC2CCN(S(=O)(=O)CC3CC3)CC2)ncc1C#N]

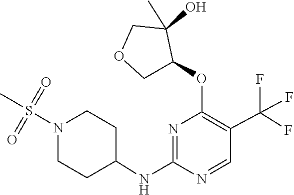 [SMILES: C[C@@]1(O)COC[C@@H]1Oc1nc(NC2CCN(S(C)(=O)=O)CC2)ncc1C(F)(F)F]

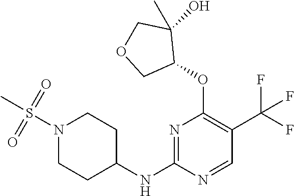 [SMILES: C[C@]1(O)COC[C@H]1Oc1nc(NC2CCN(S(C)(=O)=O)CC2)ncc1C(F)(F)F]

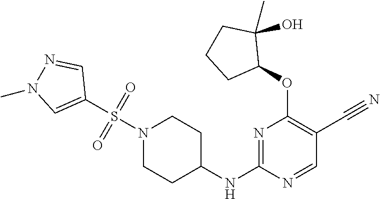 [SMILES: Cn1cc(S(=O)(=O)N2CCC(Nc3ncc(C#N)c(O[C@H]4CCC[C@@]4(C)O)n3)CC2)cn1]

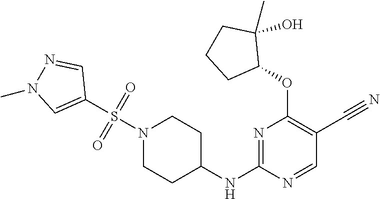 [SMILES: Cn1cc(S(=O)(=O)N2CCC(Nc3ncc(C#N)c(O[C@@H]4CCC[C@]4(C)O)n3)CC2)cn1]

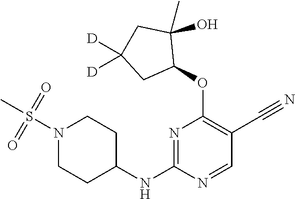 [SMILES: [2H]C1([2H])C[C@H](Oc2nc(NC3CCN(S(C)(=O)=O)CC3)ncc2C#N)[C@](C)(O)C1]

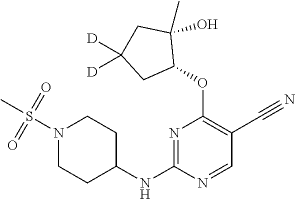 [SMILES: [2H]C1([2H])C[C@@H](Oc2nc(NC3CCN(S(C)(=O)=O)CC3)ncc2C#N)[C@@](C)(O)C1]

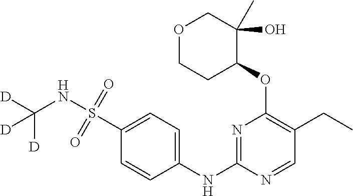 [SMILES: [2H]C([2H])([2H])NS(=O)(=O)c1ccc(Nc2ncc(CC)c(O[C@H]3CCOC[C@@]3(C)O)n2)cc1]

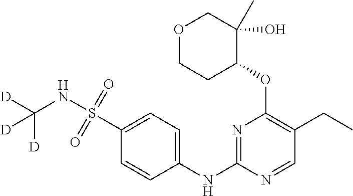 [SMILES: [2H]C([2H])([2H])NS(=O)(=O)c1ccc(Nc2ncc(CC)c(O[C@@H]3CCOC[C@]3(C)O)n2)cc1]

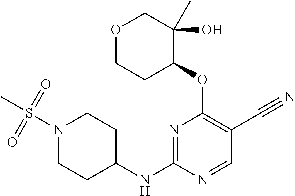 [SMILES: C[C@@]1(O)COCC[C@@H]1Oc1nc(NC2CCN(S(C)(=O)=O)CC2)ncc1C#N]

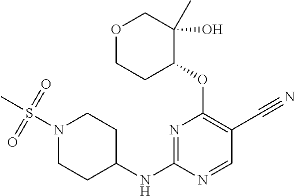 [SMILES: C[C@]1(O)COCC[C@H]1Oc1nc(NC2CCN(S(C)(=O)=O)CC2)ncc1C#N]

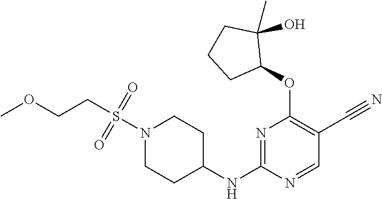 [SMILES: COCCS(=O)(=O)N1CCC(Nc2ncc(C#N)c(O[C@H]3CCC[C@@]3(C)O)n2)CC1]

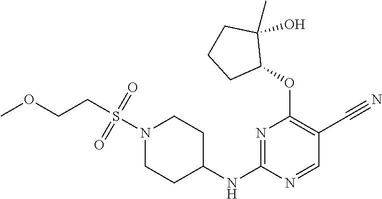 [SMILES: COCCS(=O)(=O)N1CCC(Nc2ncc(C#N)c(O[C@@H]3CCC[C@]3(C)O)n2)CC1]

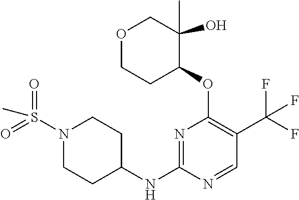 [SMILES: C[C@@]1(O)COCC[C@@H]1Oc1nc(NC2CCN(S(C)(=O)=O)CC2)ncc1C(F)(F)F]

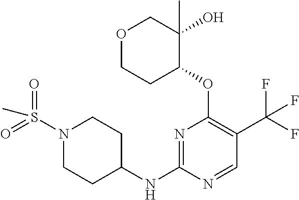 [SMILES: C[C@]1(O)COCC[C@H]1Oc1nc(NC2CCN(S(C)(=O)=O)CC2)ncc1C(F)(F)F]

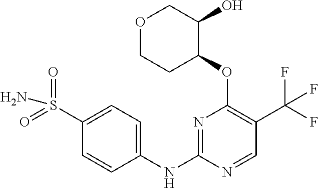 [SMILES: NS(=O)(=O)c1ccc(Nc2ncc(C(F)(F)F)c(O[C@H]3CCOC[C@H]3O)n2)cc1]

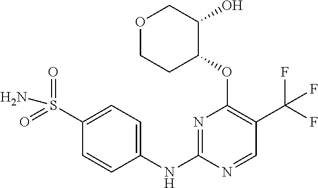 [SMILES: NS(=O)(=O)c1ccc(Nc2ncc(C(F)(F)F)c(O[C@@H]3CCOC[C@@H]3O)n2)cc1]

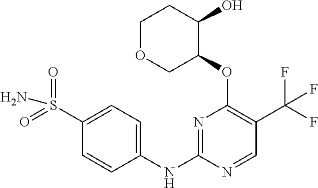 [SMILES: NS(=O)(=O)c1ccc(Nc2ncc(C(F)(F)F)c(O[C@H]3COCC[C@H]3O)n2)cc1]

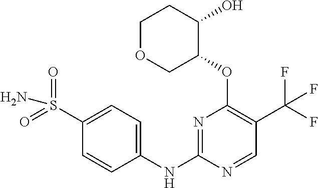 [SMILES: NS(=O)(=O)c1ccc(Nc2ncc(C(F)(F)F)c(O[C@@H]3COCC[C@@H]3O)n2)cc1]

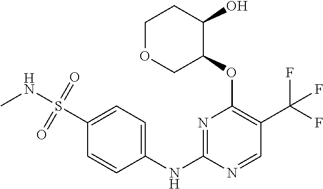 [SMILES: CNS(=O)(=O)c1ccc(Nc2ncc(C(F)(F)F)c(O[C@H]3COCC[C@H]3O)n2)cc1]

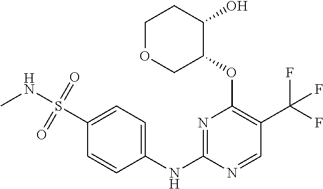 [SMILES: CNS(=O)(=O)c1ccc(Nc2ncc(C(F)(F)F)c(O[C@@H]3COCC[C@@H]3O)n2)cc1]

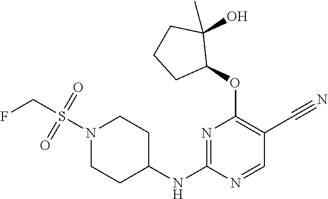 [SMILES: C[C@@]1(O)CCC[C@@H]1Oc1nc(NC2CCN(S(=O)(=O)CF)CC2)ncc1C#N]

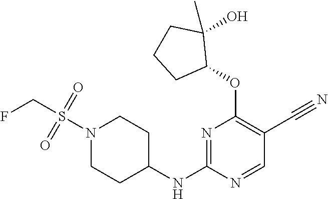 [SMILES: C[C@]1(O)CCC[C@H]1Oc1nc(NC2CCN(S(=O)(=O)CF)CC2)ncc1C#N]

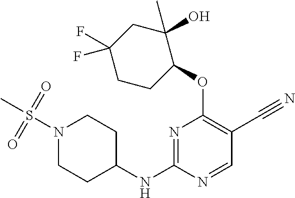 [SMILES: C[C@@]1(O)CC(F)(F)CC[C@@H]1Oc1nc(NC2CCN(S(C)(=O)=O)CC2)ncc1C#N]

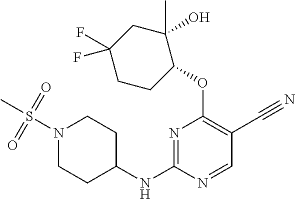 [SMILES: C[C@]1(O)CC(F)(F)CC[C@H]1Oc1nc(NC2CCN(S(C)(=O)=O)CC2)ncc1C#N]

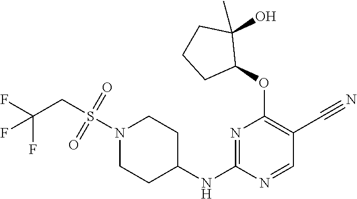 [SMILES: C[C@@]1(O)CCC[C@@H]1Oc1nc(NC2CCN(S(=O)(=O)CC(F)(F)F)CC2)ncc1C#N]

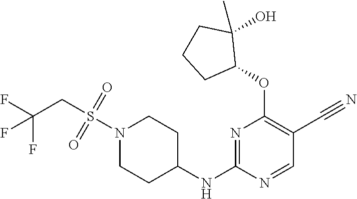 [SMILES: C[C@]1(O)CCC[C@H]1Oc1nc(NC2CCN(S(=O)(=O)CC(F)(F)F)CC2)ncc1C#N]

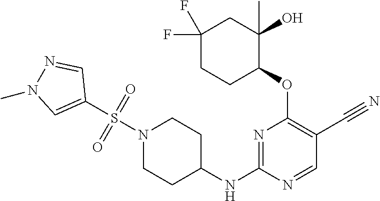 [SMILES: Cn1cc(S(=O)(=O)N2CCC(Nc3ncc(C#N)c(O[C@H]4CCC(F)(F)C[C@@]4(C)O)n3)CC2)cn1]

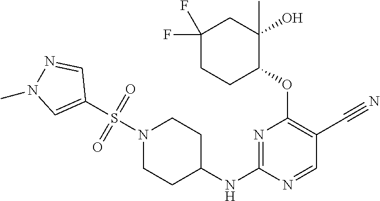 [SMILES: Cn1cc(S(=O)(=O)N2CCC(Nc3ncc(C#N)c(O[C@@H]4CCC(F)(F)C[C@]4(C)O)n3)CC2)cn1]

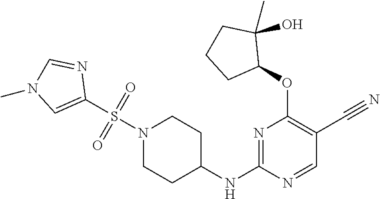 [SMILES: Cn1cnc(S(=O)(=O)N2CCC(Nc3ncc(C#N)c(O[C@H]4CCC[C@@]4(C)O)n3)CC2)c1]

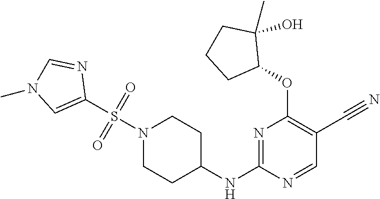 [SMILES: Cn1cnc(S(=O)(=O)N2CCC(Nc3ncc(C#N)c(O[C@@H]4CCC[C@]4(C)O)n3)CC2)c1]

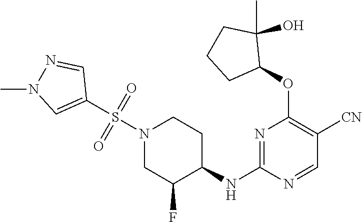 [SMILES: Cn1cc(S(=O)(=O)N2CC[C@@H](Nc3ncc(C#N)c(O[C@H]4CCC[C@@]4(C)O)n3)[C@@H](F)C2)cn1]

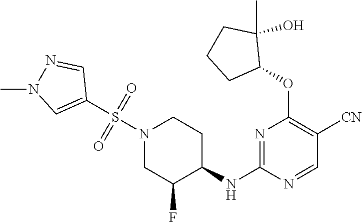 [SMILES: Cn1cc(S(=O)(=O)N2CC[C@@H](Nc3ncc(C#N)c(O[C@@H]4CCC[C@]4(C)O)n3)[C@@H](F)C2)cn1]

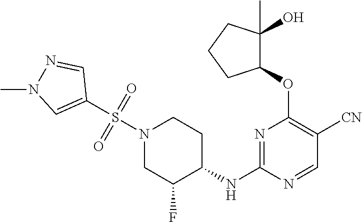 [SMILES: Cn1cc(S(=O)(=O)N2CC[C@H](Nc3ncc(C#N)c(O[C@H]4CCC[C@@]4(C)O)n3)[C@H](F)C2)cn1]

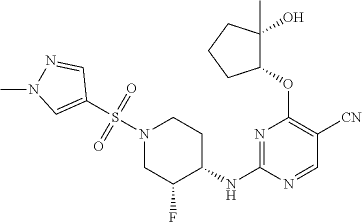 [SMILES: Cn1cc(S(=O)(=O)N2CC[C@H](Nc3ncc(C#N)c(O[C@@H]4CCC[C@]4(C)O)n3)[C@H](F)C2)cn1]

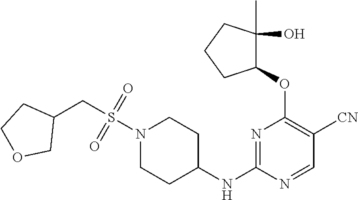 [SMILES: C[C@@]1(O)CCC[C@@H]1Oc1nc(NC2CCN(S(=O)(=O)CC3CCOC3)CC2)ncc1C#N]

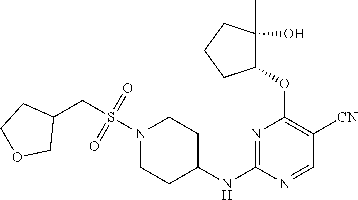 [SMILES: C[C@]1(O)CCC[C@H]1Oc1nc(NC2CCN(S(=O)(=O)CC3CCOC3)CC2)ncc1C#N]

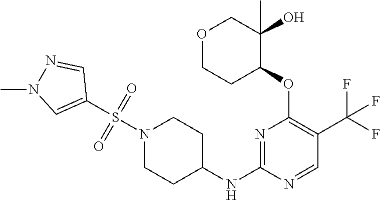 [SMILES: Cn1cc(S(=O)(=O)N2CCC(Nc3ncc(C(F)(F)F)c(O[C@H]4CCOC[C@@]4(C)O)n3)CC2)cn1]

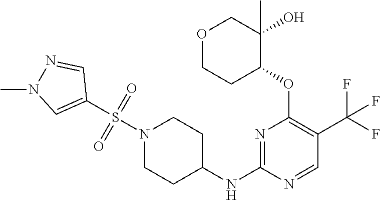 [SMILES: Cn1cc(S(=O)(=O)N2CCC(Nc3ncc(C(F)(F)F)c(O[C@@H]4CCOC[C@]4(C)O)n3)CC2)cn1]

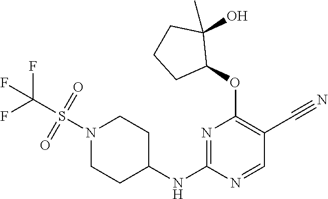 [SMILES: C[C@@]1(O)CCC[C@@H]1Oc1nc(NC2CCN(S(=O)(=O)C(F)(F)F)CC2)ncc1C#N]

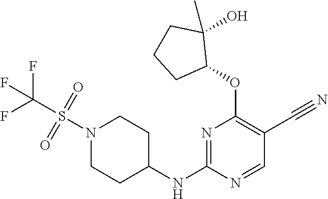 [SMILES: C[C@]1(O)CCC[C@H]1Oc1nc(NC2CCN(S(=O)(=O)C(F)(F)F)CC2)ncc1C#N]

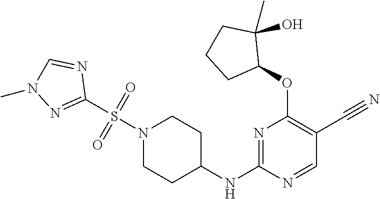 [SMILES: Cn1cnc(S(=O)(=O)N2CCC(Nc3ncc(C#N)c(O[C@H]4CCC[C@@]4(C)O)n3)CC2)n1]

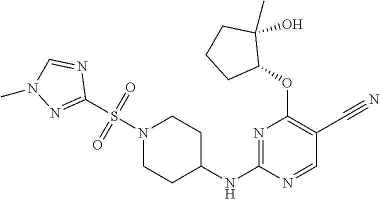 [SMILES: Cn1cnc(S(=O)(=O)N2CCC(Nc3ncc(C#N)c(O[C@@H]4CCC[C@]4(C)O)n3)CC2)n1]

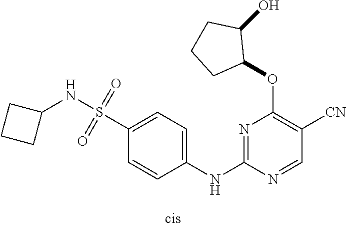 [SMILES: N#Cc1cnc(Nc2ccc(S(=O)(=O)NC3CCC3)cc2)nc1O[C@H]1CCC[C@H]1O]

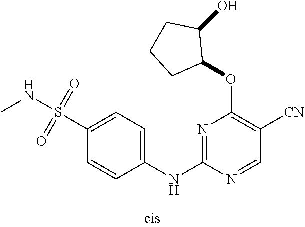 [SMILES: CNS(=O)(=O)c1ccc(Nc2ncc(C#N)c(O[C@H]3CCC[C@H]3O)n2)cc1]

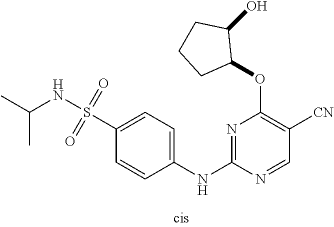 [SMILES: CC(C)NS(=O)(=O)c1ccc(Nc2ncc(C#N)c(O[C@H]3CCC[C@H]3O)n2)cc1]

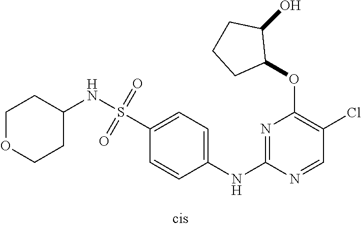 [SMILES: O=S(=O)(NC1CCOCC1)c1ccc(Nc2ncc(Cl)c(O[C@H]3CCC[C@H]3O)n2)cc1]

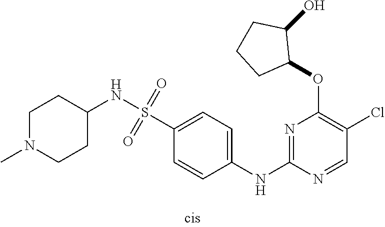 [SMILES: CN1CCC(NS(=O)(=O)c2ccc(Nc3ncc(Cl)c(O[C@H]4CCC[C@H]4O)n3)cc2)CC1]

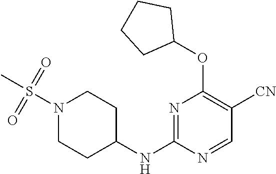 [SMILES: CS(=O)(=O)N1CCC(Nc2ncc(C#N)c(OC3CCCC3)n2)CC1]

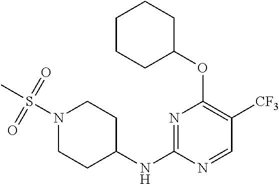 [SMILES: CS(=O)(=O)N1CCC(Nc2ncc(C(F)(F)F)c(OC3CCCCC3)n2)CC1]

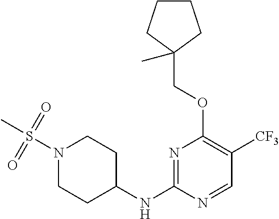 [SMILES: CC1(COc2nc(NC3CCN(S(C)(=O)=O)CC3)ncc2C(F)(F)F)CCCC1]

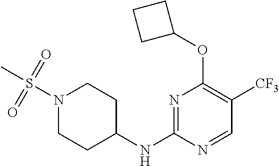 [SMILES: CS(=O)(=O)N1CCC(Nc2ncc(C(F)(F)F)c(OC3CCC3)n2)CC1]

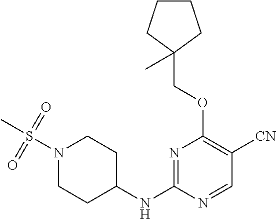 [SMILES: CC1(COc2nc(NC3CCN(S(C)(=O)=O)CC3)ncc2C#N)CCCC1]

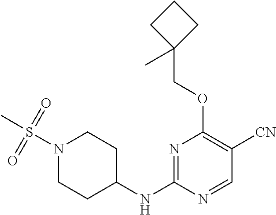 [SMILES: CC1(COc2nc(NC3CCN(S(C)(=O)=O)CC3)ncc2C#N)CCC1]

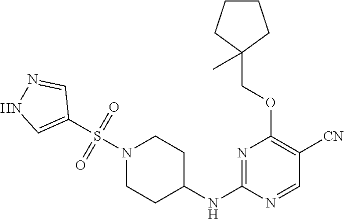 [SMILES: CC1(COc2nc(NC3CCN(S(=O)(=O)c4cn[nH]c4)CC3)ncc2C#N)CCCC1]

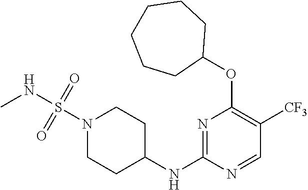 [SMILES: CNS(=O)(=O)N1CCC(Nc2ncc(C(F)(F)F)c(OC3CCCCCC3)n2)CC1]

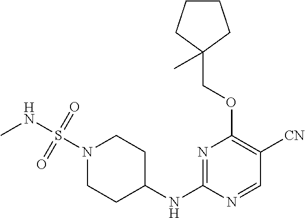 [SMILES: CNS(=O)(=O)N1CCC(Nc2ncc(C#N)c(OCC3(C)CCCC3)n2)CC1]

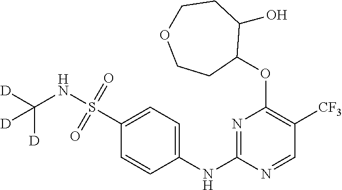 [SMILES: [2H]C([2H])([2H])NS(=O)(=O)c1ccc(Nc2ncc(C(F)(F)F)c(OC3CCOCCC3O)n2)cc1]

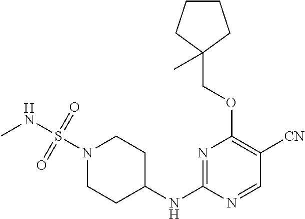 [SMILES: CNS(=O)(=O)N1CCC(Nc2ncc(C#N)c(OCC3(C)CCCC3)n2)CC1]

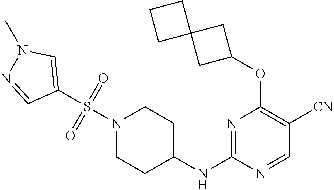 [SMILES: Cn1cc(S(=O)(=O)N2CCC(Nc3ncc(C#N)c(OC4CC5(CCC5)C4)n3)CC2)cn1]

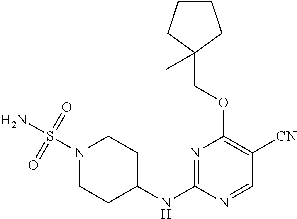 [SMILES: CC1(COc2nc(NC3CCN(S(N)(=O)=O)CC3)ncc2C#N)CCCC1]

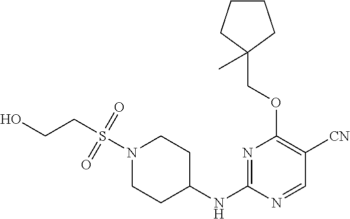 [SMILES: CC1(COc2nc(NC3CCN(S(=O)(=O)CCO)CC3)ncc2C#N)CCCC1]

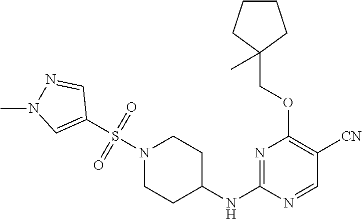 [SMILES: Cn1cc(S(=O)(=O)N2CCC(Nc3ncc(C#N)c(OCC4(C)CCCC4)n3)CC2)cn1]

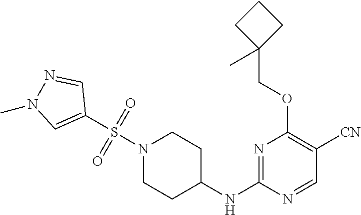 [SMILES: Cn1cc(S(=O)(=O)N2CCC(Nc3ncc(C#N)c(OCC4(C)CCC4)n3)CC2)cn1]

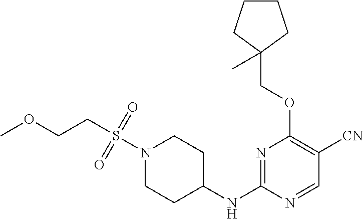 [SMILES: COCCS(=O)(=O)N1CCC(Nc2ncc(C#N)c(OCC3(C)CCCC3)n2)CC1]

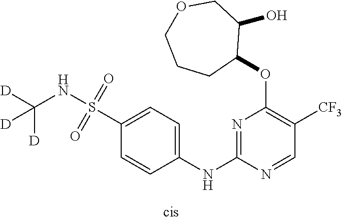 [SMILES: [2H]C([2H])([2H])NS(=O)(=O)c1ccc(Nc2ncc(C(F)(F)F)c(O[C@H]3CCCOC[C@H]3O)n2)cc1]

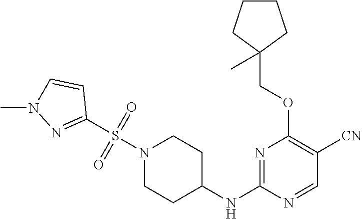 [SMILES: Cn1ccc(S(=O)(=O)N2CCC(Nc3ncc(C#N)c(OCC4(C)CCCC4)n3)CC2)n1]

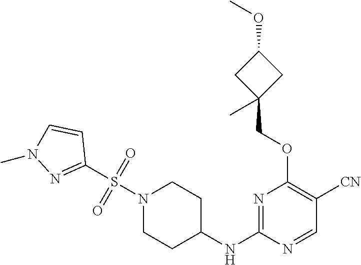 [SMILES: CO[C@H]1C[C@](C)(COc2nc(NC3CCN(S(=O)(=O)c4ccn(C)n4)CC3)ncc2C#N)C1]

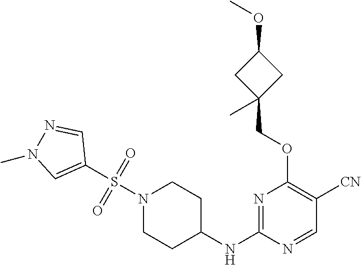 [SMILES: CO[C@H]1C[C@@](C)(COc2nc(NC3CCN(S(=O)(=O)c4cnn(C)c4)CC3)ncc2C#N)C1]

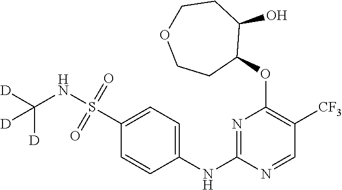 [SMILES: [2H]C([2H])([2H])NS(=O)(=O)c1ccc(Nc2ncc(C(F)(F)F)c(O[C@H]3CCOCC[C@H]3O)n2)cc1]

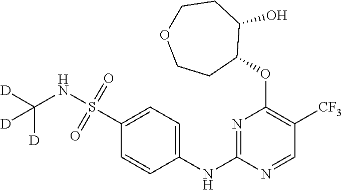 [SMILES: [2H]C([2H])([2H])NS(=O)(=O)c1ccc(Nc2ncc(C(F)(F)F)c(O[C@@H]3CCOCC[C@@H]3O)n2)cc1]

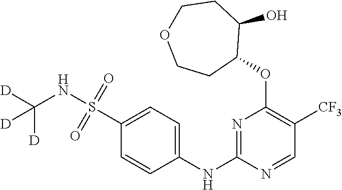 [SMILES: [2H]C([2H])([2H])NS(=O)(=O)c1ccc(Nc2ncc(C(F)(F)F)c(O[C@@H]3CCOCC[C@H]3O)n2)cc1]

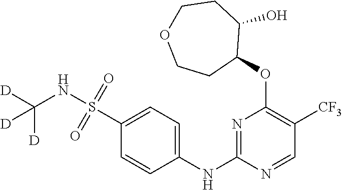 [SMILES: [2H]C([2H])([2H])NS(=O)(=O)c1ccc(Nc2ncc(C(F)(F)F)c(O[C@H]3CCOCC[C@@H]3O)n2)cc1]

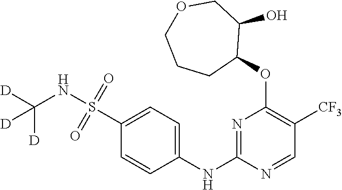 [SMILES: [2H]C([2H])([2H])NS(=O)(=O)c1ccc(Nc2ncc(C(F)(F)F)c(O[C@H]3CCCOC[C@H]3O)n2)cc1]

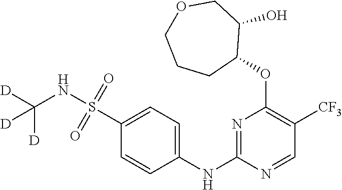 [SMILES: [2H]C([2H])([2H])NS(=O)(=O)c1ccc(Nc2ncc(C(F)(F)F)c(O[C@@H]3CCCOC[C@@H]3O)n2)cc1]

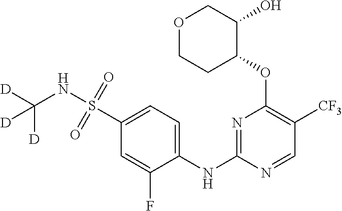 [SMILES: [2H]C([2H])([2H])NS(=O)(=O)c1ccc(Nc2ncc(C(F)(F)F)c(O[C@@H]3CCOC[C@@H]3O)n2)c(F)c1]

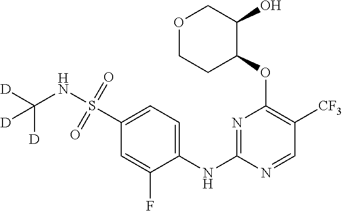 [SMILES: [2H]C([2H])([2H])NS(=O)(=O)c1ccc(Nc2ncc(C(F)(F)F)c(O[C@H]3CCOC[C@H]3O)n2)c(F)c1]

Compounds of Table 1A and 1B can exist in various stereoisomeric forms, such as individual isomer, an individual enantiomer and/or diastereomer, as applicable, or a mixture of stereoisomers, including racemic mixtures and mixtures enriched in one or more stereoisomers. In some embodiments, when applicable, a compound shown Table 1A or 1B can exist as an isolated individual enantiomer substantially free (e.g., with less than 20%, less than 10%, less than 5%, less than 1%, by weight, by HPLC or SFC area, or both, or with a non-detectable amount) of the other enantiomer. In some embodiments, when applicable, a compound shown Table 1A or 1B can also exist as a mixture of stereoisomers in any ratio, such as a racemic mixture.

In some embodiments, to the extent applicable, the genus of compounds described herein also excludes any specifically known single compounds prior to this disclosure. In some embodiments, to the extent applicable, any sub-genus or species of compounds prior to this disclosure that are entirely within a genus of compounds described herein can also be excluded from such genus herein.

Method of Synthesis

The compounds of the present disclosure can be readily synthesized by those skilled in the art in view of the present disclosure. Exemplified synthesis are also shown in the Examples section.

The synthesis of compounds of Formula I shown in Scheme 1 is illustrative. As shown in Scheme 1, compounds of Formula I can be typically prepared from a compound of S-2 via a series of coupling reactions. For example, in some embodiments, the compound of S-2 can first react with amine S-1 to form the compound of S-3. Typically, G^{1A} in S-2 is a leaving group as described herein, such as a halogen, e.g., Cl, and G^{1B} in S-1 is typically hydrogen. Conditions for coupling compounds of S-1 and S-2 include any of those conditions known for similar transformations. Exemplary conditions are shown herein in the Examples section. The compound of S-3 can then react with S-4 to form the compound of Formula I. Typically, G^{2A} in S-3 is a leaving group as described herein, such as a halogen, e.g., F, Cl, and G^{2B} in S-4 is typically hydrogen, when L² is O or NR¹⁴, or when R²-L³-L² represents a heterocyclic ring which connects to the pyridine or pyrimidine ring in Formula I via a ring nitrogen. Conditions for coupling compounds of S-3 and S-4 include any of those conditions known for similar transformations. Exemplary conditions are shown herein in the Examples section. In some embodiments, G^{2A} in S-3 can be a leaving group as described herein, such as a halogen, and G^{2B} in S-4 can be a coupling partner such as bornic acid/ester, tin, zinc, such that S-4 can react with S-3 under appropriate conditions (e.g., palladium catalyzed cross coupling reactions) to introduce the R²-L³-L² group. The variables L¹, L², L³, R¹, R², R³, R⁴, and X for the formulae in Scheme 1 include any of those described herein in any combinations. Although Scheme 1 describes one particular sequence of coupling various compounds with S-2 to provide the compound of Formula I, the present disclosure is not limited to this sequence of coupling. For example, in some embodiments, the synthetic method can start with coupling S-2 with S-4 to form the R²-L³-L² group, followed by reacting the resulting compound with a sequential coupling with S-1 and S-4 to provide the compound of Formula I. Compounds of S-2 can be commercially available and can be generally prepared according to various heteroaryl formation methods and/or subsequent transformations known in the art. The coupling partners S-1, and S-4 are generally available commercially or can be readily prepared by those skilled in the art in view of the present disclosure.

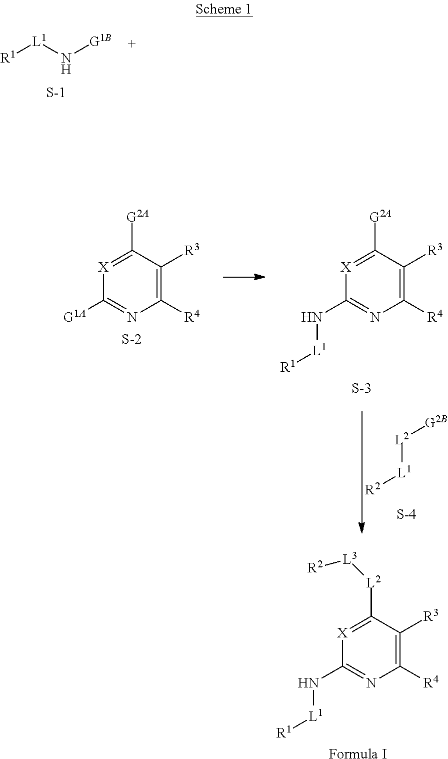

As will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in “Protective Groups in Organic Synthesis”, 4^{th} ed. P. G. M. Wuts; T. W. Greene, John Wiley, 2007, and references cited therein. The reagents for the reactions described herein are generally known compounds or can be prepared by known procedures or obvious modifications thereof. For example, many of the reagents are available from commercial suppliers such as Aldrich Chemical Co. (Milwaukee, Wisconsin, USA), Sigma (St. Louis, Missouri, USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplemental (Elsevier Science Publishers, 1989), Organic Reactions, Volumes 1-40 (John Wiley and Sons, 1991), March's Advanced Organic Chemistry, (Wiley, 7^{th} Edition), and Larock's Comprehensive Organic Transformations (Wiley-VCH, 1999), and any of available updates as of this filing.

Pharmaceutical Compositions

Certain embodiments are directed to a pharmaceutical composition comprising one or more compounds of the present disclosure.

The pharmaceutical composition can optionally contain a pharmaceutically acceptable excipient. In some embodiments, the pharmaceutical composition comprises a compound of the present disclosure (e.g., a compound of Formula I (e.g., I-1, I-2, I-3, I-4, I-5, I-2-1, I-2-1-S1, I-2-1-S2, I-2-1-S3, I-2-1-S4, I-5-1, I-5-2, I-A, I-A-1, I-A-2, I-A-3, I-A-4, I- A-5A, I-A-6A, I-A-7A, I-A-8A, I-A-9A, I-A-10A, I-A-5B, I-A-6B, I-A-7B, I-A-8B, I-A-9B, I-A-10B, or I-B), Formula II (e.g., II-A, II-1, II-2, II-1-S1, II-1-S2, II-1-S3, II-1-S4, II-2-S1, II-2-S2, II-2-S3, or II-2-S4), Examples 1-155, or any of the specific compounds disclosed in Table 1A or 1B herein, or a pharmaceutically acceptable salt thereof) and a pharmaceutically acceptable excipient. Pharmaceutically acceptable excipients are known in the art. Non-limiting suitable excipients include, for example, encapsulating materials or additives such as antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. See also Remington's The Science and Practice of Pharmacy, 21st Edition, A. R. Gennaro (Lippincott, Williams & Wilkins, Baltimore, Md., 2005; incorporated herein by reference), which discloses various excipients used in formulating pharmaceutical compositions and known techniques for the preparation thereof.

The pharmaceutical composition can include any one or more of the compounds of the present disclosure. For example, in some embodiments, the pharmaceutical composition comprises a compound of Formula I (e.g., I-1, I-2, I-3, I-4, I-5, I-2-1, I-2-1-51, 1-2-1-S2, I-2-1-S3, I-2-1-S4, I-5-1, I-5-2, I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B, I-A-8B, I-A-9B, I-A-10B, or I-B), Formula II (e.g., II-A, II-1, II-2, II-1-S1, II-1-S2, II-1-S3, II-1-S4, II-2-S1, II-2-S2, II-2-S3, or II-2-S4), Examples 1-155, or any of the specific compounds disclosed in Table 1A or 1B herein, or a pharmaceutically acceptable salt thereof, e.g., in a therapeutically effective amount. In any of the embodiments described herein, the pharmaceutical composition can comprise a therapeutically effective amount (e.g., for treating breast cancer or ovarian cancer) of a compound selected from any of Examples 1-155, or any of the specific compounds disclosed in Table 1A or 1B herein, or a pharmaceutically acceptable salt thereof. In some preferred embodiments, the pharmaceutical composition can comprise a compound selected from the compounds according to Examples 1-155 that have a CDK2/CyclinE1 IC50 level designated as “A” or “B”, preferably, “A” in Table 2 herein.

The pharmaceutical composition herein can be formulated for delivery via any of the known routes of delivery, which include but not limited to administering orally, nasally, transdermally, pulmonary, inhalationally, buccally, sublingually, intraperintoneally, subcutaneously, intramuscularly, intravenously, rectally, intrapleurally, intrathecally or parenterally.

In some embodiments, the pharmaceutical composition can be formulated for oral administration. The oral formulations can be presented in discrete units, such as capsules, pills, cachets, lozenges, or tablets, each containing a predetermined amount of the active compound; as a powder or granules; as a solution or a suspension in an aqueous or non-aqueous liquid; or as an oil-in-water or water-in-oil emulsion. Excipients for the preparation of compositions for oral administration are known in the art. Non-limiting suitable excipients include, for example, agar, alginic acid, aluminum hydroxide, benzyl alcohol, benzyl benzoate, 1,3-butylene glycol, carbomers, castor oil, cellulose, cellulose acetate, cocoa butter, corn starch, corn oil, cottonseed oil, cross-povidone, diglycerides, ethanol, ethyl cellulose, ethyl laureate, ethyl oleate, fatty acid esters, gelatin, germ oil, glucose, glycerol, groundnut oil, hydroxypropylmethyl cellulose, isopropanol, isotonic saline, lactose, magnesium hydroxide, magnesium stearate, malt, mannitol, monoglycerides, olive oil, peanut oil, potassium phosphate salts, potato starch, povidone, propylene glycol, Ringer's solution, safflower oil, sesame oil, sodium carboxymethyl cellulose, sodium phosphate salts, sodium lauryl sulfate, sodium sorbitol, soybean oil, stearic acids, stearyl fumarate, sucrose, surfactants, talc, tragacanth, tetrahydrofurfuryl alcohol, triglycerides, water, and mixtures thereof.

In some embodiments, the pharmaceutical composition is formulated for parenteral administration (such as intravenous injection or infusion, subcutaneous or intramuscular injection). The parenteral formulations can be, for example, an aqueous solution, a suspension, or an emulsion. Excipients for the preparation of parenteral formulations are known in the art. Non-limiting suitable excipients include, for example, 1,3-butanediol, castor oil, corn oil, cottonseed oil, dextrose, germ oil, groundnut oil, liposomes, oleic acid, olive oil, peanut oil, Ringer's solution, safflower oil, sesame oil, soybean oil, U.S.P. or isotonic sodium chloride solution, water and mixtures thereof.

Compounds of the present disclosure can be used alone, in combination with each other, or in combination with one or more additional therapeutic agents, e.g., in combination with an additional anticancer therapeutic agent, such as mitotic inhibitors, alkylating agents, antimetabolites, antitumor antibiotics, anti-angiogenesis agents, topoisomerase I and II inhibitors, plant alkaloids, hormonal agents and antagonists, growth factor inhibitors, radiation, signal transduction inhibitors, such as inhibitors of protein tyrosine kinases and/or serine/threonine kinases, cell cycle inhibitors, biological response modifiers, enzyme inhibitors, antisense oligonucleotides or oligonucleotide derivatives, cytotoxics, immuno-oncology agents, and the like. In some embodiments, one or more compounds of the present disclosure can be used in combination with one or more targeted agents, such as inhibitors of PI3 kinase, mTOR, PARP, IDO, TDO, ALK, ROS, MEK, VEGF, FLT3, AXL, ROR2, EGFR, FGFR, Src/Abl, RTK/Ras, Myc, Raf, PDGF, AKT, c-Kit, erbB, CDK4/CDK6, CDK5, CDK7, CDK9, SMO, CXCR4, HER2, GLS1, EZH2 or Hsp90, or immunomodulatory agents, such as PD-1 or PD-L1 antagonists, OX40 agonists or 4-1BB agonists. In some embodiments, one or more compounds of the present disclosure can be used in combination with a standard of care agent, such as tamoxifen, docetaxel, paclitaxel, cisplatin, capecitabine, gemcitabine, vinorelbine, exemestane, letrozole, fulvestrant, anastrozole or trastuzumab. Suitable additional anticancer therapeutic agent include any of those known in the art, such as those approved for the appropriate cancer by a regulatory agency such as the U.S. Food and Drug Administration. Some examples of suitable additional anticancer therapeutic agents also include those described in WO2020/157652, US2018/0044344, WO2008/122767, etc., the content of each of which is herein incorporated by reference in its entireties.

When used in combination with one or more additional therapeutic agents, compounds of the present disclosure or pharmaceutical compositions herein can be administered to the subject either concurrently or sequentially in any order with such additional therapeutic agents. In some embodiments, the pharmaceutical composition can comprise one or more compounds of the present disclosure and the one or more additional therapeutic agents in a single composition. In some embodiments, the pharmaceutical composition comprising one or more compounds of the present disclosure can be included in a kit which also comprises a separate pharmaceutical composition comprising the one or more additional therapeutic agents.

The pharmaceutical composition can include various amounts of the compounds of the present disclosure, depending on various factors such as the intended use and potency and selectivity of the compounds. In some embodiments, the pharmaceutical composition comprises a therapeutically effective amount of a compound of the present disclosure. In some embodiments, the pharmaceutical composition comprises a therapeutically effective amount of the compound of the present disclosure and a pharmaceutically acceptable excipient. As used herein, a therapeutically effective amount of a compound of the present disclosure is an amount effective to treat a disease or disorder as described herein, such as breast cancer or ovarian cancer, which can depend on the recipient of the treatment, the disorder, condition or disease being treated and the severity thereof, the composition containing the compound, the time of administration, the route of administration, the duration of treatment, the compound potency, its rate of clearance and whether or not another drug is co-administered.

Method of Treatment/Use

Compounds of the present disclosure have various utilities. For example, compounds of the present disclosure can be used as therapeutic active substances for the treatment and/or prophylaxis of a CDK2-mediated disease or disorder. Accordingly, some embodiments of the present disclosure are also directed to methods of using one or more compounds of the present disclosure or pharmaceutical compositions herein for treating or preventing a CDK2-mediated disease or disorder in a subject in need thereof, such as for treating cancer in a subject in need thereof.

In some embodiments, the present disclosure provides a method of inhibiting abnormal cell growth in a subject in need thereof, comprising administering to the subject a therapeutically effective amount of a compound of the present disclosure or a pharmaceutical composition described herein. In some embodiments, the abnormal cell growth is cancer characterized by amplification or overexpression of cyclin E1 (CCNE1) and/or cyclin E2 (CCNE2). In some embodiments, the subject is identified as having a cancer characterized by amplification or overexpression of CCNE1 and/or CCNE2.

In some embodiments, the present disclosure also provides a method of inhibiting CDK activity in a subject or biological sample. In some embodiments, the present disclosure provides a method of inhibiting CDK2 activity in a subject or biological sample, which comprises contacting the subject or biological sample with an effective amount of the compound of the present disclosure (e.g., a compound of Formula I (e.g., I-1, I-2, I-3, I-4, I-5, I-2-1, I-2-1-S1, I-2-1-S2, I-2-1-S3, I-2-1-S4, I-5-1, I-5-2, I-A, I-A-1, I-A-2, I-A-3, I-A-4, I- A-5A, I-A-6A, I-A-7A, I-A-8A, I-A-9A, I-A-10A, I-A-5B, I-A-6B, I-A-7B, I-A-8B, I-A-9B, I-A-10B, or I-B), Formula II (e.g., II-A, II-1, II-2, II-1-S1, II-1-S2, II-1-S3, II-1-S4, II-2-S1, II-2-S2, II-2-S3, or II-2-S4), any of Examples 1-155, or any of the specific compounds disclosed in Table 1A or 1B herein, or a pharmaceutically acceptable salt thereof) or a pharmaceutical composition described herein.

In some embodiments, the present disclosure provides a method of treating or preventing a CDK mediated, in particular CDK2-mediated disease or disorder in a subject in need thereof. In some embodiments, the method comprises administering to the subject an effective amount of a compound of the present disclosure (e.g., a compound of Formula I (e.g., I-1, I-2, I-3, I-4, I-5, I-2-1, I-2-1-S1, I-2-1-S2, I-2-1-S3, I-2-1-S4, I-5-1, I-5-2, I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B, I-A-8B, I-A-9B, I-A-10B, or I-B), Formula II (e.g., II-A, II-1, II-2, II-1-S1, II-1-S2, II-1-S3, II-1-S4, II-2-S1, II-2-S2, II-2-S3, or II-2-S4), any of Examples 1-155, or any of the specific compounds disclosed in Table 1A or 1B herein, or a pharmaceutically acceptable salt thereof) or an effective amount of a pharmaceutical composition described herein. In some embodiments, the CDK2-mediated disease or disorder is cancer. In some embodiments, the cancer is characterized by amplification or overexpression of CCNE1 and/or CCNE2

In some embodiments, the present disclosure also provides a method of treating or preventing cancer in a subject in need thereof, which comprises administering to the subject an effective amount of a compound of the present disclosure (e.g., a compound of Formula I (e.g., I-1, I-2, I-3, I-4, I-5, I-2-1, I-2-1-S1, I-2-1-S2, I-2-1-S3, I-2-1-S4, I-5-1, I-5-2, I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B, I-A-8B, I-A-9B, I-A-10B, or I-B), Formula II (e.g., II-A, II-1, II-2, II-1-S1, II-1-S2, II-1-S3, II-1-S4, II-2-S1, II-2-S2, II-2-S3, or II-2-S4), Examples 1-155, or any of the specific compounds disclosed in Table 1A or 1B herein, or a pharmaceutically acceptable salt thereof) or an effective amount of a pharmaceutical composition described herein. In some embodiments, the cancer is characterized by amplification or overexpression of CCNE1 and/or CCNE2. In some embodiments, the subject is identified as having a cancer characterized by amplification or overexpression of CCNE1 and/or CCNE2. In some embodiments, the cancer is selected from breast cancer, ovarian cancer, bladder cancer, uterine cancer, prostate cancer, lung cancer (including NSCLC, SCLC, squamous cell carcinoma or adenocarcinoma), esophageal cancer, head and neck cancer, colorectal cancer, kidney cancer (including RCC), liver cancer (including HCC), pancreatic cancer, stomach (i.e., gastric) cancer, thyroid cancer, and combinations thereof. In some embodiments of the methods herein, the cancer is breast cancer, ovarian cancer, bladder cancer, uterine cancer, prostate cancer, lung cancer, esophageal cancer, liver cancer, pancreatic cancer and/or stomach cancer.

In some embodiments of the methods herein, the cancer is breast cancer, such as ER-positive/HR-positive, HER2-negative breast cancer; ER-positive/HR-positive, HER2-positive breast cancer; triple negative breast cancer (TNBC); or inflammatory breast cancer. In some embodiments, the breast cancer can be endocrine resistant breast cancer, trastuzumab resistant breast cancer, or breast cancer demonstrating primary or acquired resistance to CDK4/CDK6 inhibition. In some embodiments, the breast cancer can be advanced or metastatic breast cancer. In some embodiments, the breast cancer described herein is characterized by amplification or overexpression of CCNE1 and/or CCNE2.

In some embodiments of the methods herein, the cancer is ovarian cancer. In some embodiments, the ovarian cancer is characterized by amplification or overexpression of CCNE1 and/or CCNE2.

In some embodiments of the methods herein, the cancer is blood cancer such as leukemia. In some embodiments of the methods herein, the cancer is chronic lymphocytic leukemia, such as relapsed or refractory Chronic Lymphocytic Leukemia (CLL).

In some embodiments of the methods herein, the cancer is acute myeloid leukemia. In some embodiments of the methods herein, the cancer is relapsed or refractory Acute Myeloid Leukemia or Myelodysplastic Syndromes.

In any of the embodiments described herein, unless otherwise specified or contradictory, the cancer herein can be characterized by amplification or overexpression of CCNE1 and/or CCNE2.

In some embodiments, the present disclosure also provides a method of treating breast cancer in a subject in need thereof, which comprises administering to the subject a therapeutically effective amount of a compound of the present disclosure (e.g., a compound of Formula I (e.g., I-1, T-2, T-3, T-4, T-5, T-2-1, T-2-1-S1, T-2-1-S2, T-2-1-S3, T-2-1-S4, T-5-1, I-5-2, I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B, I-A-8B, I-A-9B, I-A-10B, or I-B), Formula II (e.g., II-A, II-1, II-2, II-1-S1, II-1-S2, II-1-S3, II-1-S4, II-2-S1, II-2-S2, II-2-S3, or 11-2-S4), any of Examples 1-155, or any of the specific compounds disclosed in Table 1A or 1B herein, or a pharmaceutically acceptable salt thereof) or an effective amount of a pharmaceutical composition described herein. In some embodiments, the breast cancer is selected from ER-positive/HR-positive, HER2-negative breast cancer; ER-positive/HR-positive, HER2-positive breast cancer; triple negative breast cancer (TNBC); and inflammatory breast cancer. In some embodiments, the breast cancer is selected from endocrine resistant breast cancer, trastuzumab resistant breast cancer, or breast cancer demonstrating primary or acquired resistance to CDK4/CDK6 inhibition. In some embodiments, the breast cancer is advanced or metastatic breast cancer. In some embodiments, the breast cancer is characterized by amplification or overexpression of CCNE1 and/or CCNE2.

In some embodiments, the present disclosure also provides a method of treating ovarian cancer in a subject in need thereof, which comprises administering to the subject a therapeutically effective amount of a compound of the present disclosure (e.g., a compound of Formula I (e.g., I-1, I-2, I-3, I-4, I-5, I-2-1, I-2-1-S1, I-2-1-52, I-2-1-53, I-2-1-54, I-5-1, I-5-2, I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B, I-A-8B, I-A-9B, I-A-10B, or I-B), Formula II (e.g., II-A, II-1, II-2, II-1-S1, II-1-S2, II-1-S3, II-1-S4, II-2-S1, II-2-S2, II-2-S3, or 11-2-S4), any of Examples 1-155, or any of the specific compounds disclosed in Table 1A or 1B herein, or a pharmaceutically acceptable salt thereof) or an effective amount of a pharmaceutical composition described herein. In some embodiments, the ovarian cancer is characterized by amplification or overexpression of CCNE1 and/or CCNE2.

In some embodiments, the present disclosure also provides a method of treating leukemia in a subject in need thereof, which comprises administering to the subject a therapeutically effective amount of a compound of the present disclosure (e.g., a compound of Formula I (e.g., I-1, I-2, I-3, I-4, I-5, I-2-1, I-2-1-S1, I-2-1-52, I-2-1-53, I-2-1-54, I-5-1, I-5-2, I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B, I-A-8B, I-A-9B, I-A-10B, or I-B), Formula II (e.g., II-A, II-1, II-2, II-1-S1, II-1-S2, II-1-S3, II-1-S4, II-2-S1, II-2-S2, II-2-S3, or 11-2-S4), any of Examples 1-155, or any of the specific compounds disclosed in Table 1A or 1B herein, or a pharmaceutically acceptable salt thereof) or an effective amount of a pharmaceutical composition described herein. In some embodiments, the leukemia is characterized by amplification or overexpression of CCNE1 and/or CCNE2.

In some embodiments, the present disclosure also provides a method of treating chronic lymphocytic leukemia, such as relapsed or refractory Chronic Lymphocytic Leukemia (CLL), in a subject in need thereof, which comprises administering to the subject a therapeutically effective amount of a compound of the present disclosure (e.g., a compound of Formula I (e.g., I-1, I-2, I-3, I-4, I-5, I-2-1, I-2-1-S1, I-2-1-52, I-2-1-53, I-2-1-54, I-5-1, I-5-2, I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B, I-A-8B, I-A-9B, I-A-10B, or I-B), Formula II (e.g., II-A, II-1, II-2, II-1-S1, II-1-S2, II-1-S3, II-1-S4, II-2-S1, II-2-S2, II-2-S3, or 11-2-S4), any of Examples 1-155, or any of the specific compounds disclosed in Table 1A or 1B herein, or a pharmaceutically acceptable salt thereof) or an effective amount of a pharmaceutical composition described herein.

In some embodiments, the present disclosure also provides a method of treating acute myeloid leukemia, such as relapsed or refractory Acute Myeloid Leukemia, in a subject in need thereof, which comprises administering to the subject a therapeutically effective amount of a compound of the present disclosure (e.g., a compound of Formula I (e.g., I-1, I-2, I-3, I-4, I-5, I-2-1, I-2-1-S1, I-2-1-S2, I-2-1-S3, I-2-1-S4, I-5-1, I-5-2, I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B, I-A-8B, I-A-9B, I-A-10B, or I-B), Formula II (e.g., II-A, II-1, II-2, II-1-S1, II-1-S2, II-1-S3, II-1-S4, II-2-S1, II-2-S2, II-2-S3, or II-2-S4), any of Examples 1-155, or any of the specific compounds disclosed in Table 1A or 1B herein, or a pharmaceutically acceptable salt thereof) or an effective amount of a pharmaceutical composition described herein.

In some embodiments, the present disclosure also provides a method of treating Myelodysplastic Syndromes in a subject in need thereof, which comprises administering to the subject a therapeutically effective amount of a compound of the present disclosure (e.g., a compound of Formula I (e.g., I-1, I-2, I-3, I-4, I-5, I-2-1, I-2-1-S1, I-2-1-S2, I-2-1-S3, I-2-1-S4, I-5-1, I-5-2, I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B, I-A-8B, I-A-9B, I-A-10B, or I-B), Formula II (e.g., II-A, II-1, II-2, II-1-S1, II-1-S2, II-1-S3, II-1-S4, II-2-S1, II-2-S2, II-2-S3, or II-2-S4), any of Examples 1-155, or any of the specific compounds disclosed in Table 1A or 1B herein, or a pharmaceutically acceptable salt thereof) or an effective amount of a pharmaceutical composition described herein.

In some preferred embodiments, the compound of the present disclosure for the methods herein has a CDK2/CyclinE1 IC50 of less than 100 nM, more preferably, less than 10 nM, measured/calculated according to the Biological Example 1 herein. In some preferred embodiments, the compound of the present disclosure for the methods herein is selected from the compounds according to Examples 1-155 that have a CDK2/CyclinE1 IC50 level designated as “A” or “B”, preferably “A”, in Table 2 herein.

The administering in the methods herein is not limited to any particular route of administration. For example, in some embodiments, the administering can be orally, nasally, transdermally, pulmonary, inhalationally, buccally, sublingually, intraperintoneally, subcutaneously, intramuscularly, intravenously, rectally, intrapleurally, intrathecally and parenterally. In some embodiments, the administering is orally. In some embodiments, the administering is a parenteral injection, such as an intraveneous injection.

Compounds of the present disclosure can be used as a monotherapy or in a combination therapy. In some embodiments according to the methods described herein, one or more compounds of the present disclosure can be administered as the only active ingredient(s). In some embodiments according to the methods described herein, one or more compounds of the present disclosure can also be co-administered with an additional therapeutic agent, either concurrently or sequentially in any order, to the subject in need thereof. The additional therapeutic agent can typically be an additional anticancer therapeutic agent, such as mitotic inhibitors, alkylating agents, antimetabolites, antitumor antibiotics, anti-angiogenesis agents, topoisomerase I and II inhibitors, plant alkaloids, hormonal agents and antagonists, growth factor inhibitors, radiation, signal transduction inhibitors, such as inhibitors of protein tyrosine kinases and/or serine/threonine kinases, cell cycle inhibitors, biological response modifiers, enzyme inhibitors, antisense oligonucleotides or oligonucleotide derivatives, cytotoxics, immuno-oncology agents, and the like. In some embodiments, the additional anticancer agent is an endocrine agent, such as an aromatase inhibitor, a SERD or a SERM. In some embodiments, one or more compounds of the present disclosure can be administered in combination with one or more targeted agents, such as inhibitors of PI3 kinase, mTOR, PARP, IDO, TDO, ALK, ROS, MEK, VEGF, FLT3, AXL, ROR2, EGFR, FGFR, Src/Abl, RTK/Ras, Myc, Raf, PDGF, AKT, c-Kit, erbB, CDK4/CDK6, CDK5, CDK7, CDK9, SMO, CXCR4, HER2, GLS1, EZH2 or Hsp90, or immunomodulatory agents, such as PD-1 or PD-L1 antagonists, OX40 agonists or 4-1BB agonists. In some embodiments, one or more compounds of the present disclosure can be administered administered in combination with a standard of care agent, such as tamoxifen, docetaxel, paclitaxel, cisplatin, capecitabine, gemcitabine, vinorelbine, exemestane, letrozole, fulvestrant, anastrozole or trastuzumab. Suitable additional anticancer therapeutic agent include any of those known in the art, such as those approved for the appropriate cancer by a regulatory agency such as the U.S. Food and Drug Administration. Some examples of suitable additional anticancer therapeutic agents also include those described in WO2020/157652, US2018/0044344, WO2008/122767, etc., the contents of each of which is incorporated by reference herein in their entirety.

Dosing regimen including doses for the methods described herein can vary and be adjusted, which can depend on the recipient of the treatment, the disorder, condition or disease being treated and the severity thereof, the composition containing the compound, the time of administration, the route of administration, the duration of treatment, the compound potency, its rate of clearance and whether or not another drug is co-administered.

### Definitions

It is meant to be understood that proper valences are maintained for all moieties and combinations thereof.

It is also meant to be understood that a specific embodiment of a variable moiety herein can be the same or different as another specific embodiment having the same identifier.

Suitable groups for the variables in compounds of Formula I or II, or a subformula thereof, as applicable, are independently selected. Non-limiting useful groups for the variables in compounds of Formula I or II, or a subformula thereof, as applicable, include any of the respective groups, individually or in any combination, as shown in the Examples or in the specific compounds described in Table 1A or 1B herein. Using variable R¹ as an example, in some embodiments, compounds of Formula I or II can include a R¹ group according to any of the R¹ groups shown in the Examples or in the specific compounds described in Table 1A or 1B herein, without regard to the other variables shown in the specific compounds. In some embodiments, compounds of Formula I or II can include a R¹ group according to any of the R¹ groups shown in the Examples or in the specific compounds described in Table 1A or 1B herein in combination at least one other variable (e.g, L¹) according to the Examples or the specific compounds described in Table 1A or 1B herein, wherein the R¹ and at least one other variable can derive from the same compound or a different compound. Any of such combinations are contemplated and within the scope of the present disclosure.

The described embodiments of the present disclosure can be combined. Such combination is contemplated and within the scope of the present disclosure. For example, it is contemplated that the definition(s) of any one or more of L¹, L², L³, R¹, R², R³, R⁴, and X of Formula I (e.g., Formula I-1, I-2, I-3, I-4, I-5, I-2-1, I-2-1-S1, I-2-1-52, I-2-1-53, I-2-1-54, I-5-1, I-5-2, I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B, I-A-8B, I-A-9B, I-A-10B, or I-B) can be combined with the definition of any one or more of the other(s) of L¹, L², L³, R¹, R², R³, R⁴, and X, as applicable, and the resulted compounds from the combination are within the scope of the present disclosure.

The symbol, [symbol] , whether utilized as a bond or displayed perpendicular to (or otherwise crossing) a bond, indicates the point at which the displayed moiety is attached to the remainder of the molecule. It should be noted that the immediately connected group or groups maybe shown beyond the symbol, [symbol] , to indicate connectivity, as would be understood by those skilled in the art.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987. The disclosure is not intended to be limited in any manner by the exemplary listing of substituents described herein.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high performance liquid chromatography (HPLC), chiral supercritical fluid chromatograph (SFC), and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, IN 1972). The disclosure additionally encompasses compounds described herein as individual isomers substantially free of other isomers, and alternatively, as mixtures of various isomers including racemic mixtures. When a stereochemistry is specifically drawn, unless otherwise contradictory from context, it should be understood that with respect to that particular chiral center or axial chirality, the compound can exist predominantly as the as-drawn stereoisomer, such as with less than 20%, less than 10%, less than 5%, less than 1%, by weight, by HPLC or SFC area, or both, or with a non-detectable amount of the other stereoisomer(s). The presence and/or amounts of stereoisomers can be determined by those skilled in the art in view of the present disclosure, including through the use of a chiral HPLC or chiral SFC. As understood by those skilled in the art, when a “*” is shown in the chemical structures herein, unless otherwise contradictory from context, it is to designate that the corresponding chiral center is enantiomerically pure or enriched in either of the configurations or is enantiomerically pure or enriched in the as-dawn configuration, such as with less than 20%, less than 10%, less than 5%, less than 1%, by weight, by HPLC or SFC area, or both, or with a non-detectable amount of the other stereoisomer(s). Also, when no stereochemistry is specifically drawn, and no “*” is used in the chemical structures, unless otherwise contradictory from context, it should be understood that such structures include the corresponding compound in any stereoisomeric forms, including individual isomers substantially free of other isomers and mixtures of various isomers including racemic mixtures.

When a range of values is listed, it is intended to encompass each value and sub-range within the range. For example “C₁₋₆” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄-s, and C₅₋₆.

As used herein, the term “compound(s) of the present disclosure” refers to any of the compounds described herein according to Formula I (e.g., I-1, I-2, I-3, I-4, I-5, I-2-1, I-2-1-S1, I-2-1-S2, I-2-1-S3, I-2-1-S4, I-5-1, I-5-2, I-A, I-A-1, I-A-2, I-A-3, I-A-4, I-A-5A, I-A-6A, I-A-7A, I-A-8A, I-A-9A, I-A-10A, I-A-5B, I-A-6B, I-A-7B, I-A-8B, I-A-9B, I-A-10B, or I-B), Formula II (e.g., II-A, II-1, II-2, II-1-S1, II-1-S2, II-1-S3, II-1-S4, II-2-S1, II-2-S2, II-2-S3, or II-2-S4), any of Examples 1-155, or any of the specific compounds disclosed in Table 1A or 1B herein, isotopically labeled compound(s) thereof (such as a deuterated analog wherein one or more of the hydrogen atoms is/are substituted with a deuterium atom with an abundance above its natural abundance, e.g., a CD₃ analog when the compound has a CH₃ group), possible regioisomers, possible geometric isomers, possible stereoisomers thereof (including diastereoisomers, enantiomers, and racemic mixtures), tautomers thereof, conformational isomers thereof, pharmaceutically acceptable esters thereof, and/or possible pharmaceutically acceptable salts thereof (e.g., acid addition salt such as HCl salt or base addition salt such as Na salt). To be clear, compounds of Examples 1-155 refer to the compounds in the Examples section labeled with an integer only, such as 1, 2, etc. up to 155, or when applicable, may be additionally followed by labels “a”, “b”, “c”, or “d” for the corresponding stereoisomers. See e.g., Illustration 1-23 and Table A herein. Collectively, Examples 1-155 should be understood as including Example Nos. 1-155, as well as those designated with an example number followed by “a”, “b”, “c”, or “d”. Exemplified synthesis and characterizations of Examples 1-155 are shown in the Examples section. Detailed exemplified procedures were shown in the Illustration examples, e.g., 1-23. Hydrates and solvates of the compounds of the present disclosure are considered compositions of the present disclosure, wherein the compound(s) is in association with water or solvent, respectively.

Compounds of the present disclosure can exist in isotope-labeled or -enriched form containing one or more atoms having an atomic mass or mass number different from the atomic mass or mass number most abundantly found in nature. Isotopes can be radioactive or non-radioactive isotopes. Isotopes of atoms such as hydrogen, carbon, phosphorous, sulfur, fluorine, chlorine, and iodine include, but are not limited to ²H, ³H, ¹³C, ¹⁴C ¹⁵N, ¹⁸O, ³²P, ³⁵S ¹⁸F, ³⁶Cl, and ¹²⁵I. Compounds that contain other isotopes of these and/or other atoms are within the scope of this invention.

As used herein, the phrase “administration” of a compound, “administering” a compound, or other variants thereof means providing the compound or a prodrug of the compound to the individual in need of treatment.

As used herein, the term “alkyl” as used by itself or as part of another group refers to a straight- or branched-chain aliphatic saturated hydrocarbon. In some embodiments, the alkyl can include one to twelve carbon atoms (i.e., C₁₋₁₂ alkyl) or the number of carbon atoms designated. In one embodiment, the alkyl group is a straight chain C₁₋₁₀ alkyl group. In another embodiment, the alkyl group is a branched chain C₃₋₁₀ alkyl group. In another embodiment, the alkyl group is a straight chain C₁₋₆ alkyl group. In another embodiment, the alkyl group is a branched chain C₃₋₆ alkyl group. In another embodiment, the alkyl group is a straight chain C₁₋₄ alkyl group. For example, a C₁₋₄ alkyl group includes methyl, ethyl, propyl (n-propyl), isopropyl, butyl (n-butyl), sec-butyl, tert-butyl, and iso-butyl. As used herein, the term “alkylene” as used by itself or as part of another group refers to a divalent radical derived from an alkyl group. For example, non-limiting straight chain alkylene groups include —CH₂—CH₂—CH₂—CH₂—, —CH₂—CH₂—CH₂—, —CH₂—CH₂—, and the like.

As used herein, the term “alkenyl” as used by itself or as part of another group refers to a straight- or branched-chain aliphatic hydrocarbon containing one or more, for example, one, two or three carbon-to-carbon double bonds. In one embodiment, the alkenyl group is a C₂₋₆ alkenyl group. In another embodiment, the alkenyl group is a C₂₋₄ alkenyl group. Non-limiting exemplary alkenyl groups include ethenyl, propenyl, isopropenyl, butenyl, sec-butenyl, pentenyl, and hexenyl.

As used herein, the term “alkynyl” as used by itself or as part of another group refers to a straight- or branched-chain aliphatic hydrocarbon containing one or more, for example, one to three carbon-to-carbon triple bonds. In one embodiment, the alkynyl has one carbon-carbon triple bond. In one embodiment, the alkynyl group is a C₂₋₆ alkynyl group. In another embodiment, the alkynyl group is a C₂₋₄ alkynyl group. Non-limiting exemplary alkynyl groups include ethynyl, propynyl, butynyl, 2-butynyl, pentynyl, and hexynyl groups.

As used herein, the term “alkoxy” as used by itself or as part of another group refers to a radical of the formula OR^{a1}, wherein R^{a1} is an alkyl.

As used herein, the term “cycloalkoxy” as used by itself or as part of another group refers to a radical of the formula OR^{a1}, wherein R^{a1} is a cycloalkyl.

As used herein, the term “haloalkyl” as used by itself or as part of another group refers to an alkyl substituted with one or more fluorine, chlorine, bromine and/or iodine atoms. In preferred embodiments, the haloalkyl is an alkyl group substituted with one, two, or three fluorine atoms. In one embodiment, the haloalkyl group is a C₁₋₁₀ haloalkyl group. In one embodiment, the haloalkyl group is a C₁₋₆ haloalkyl group. In one embodiment, the haloalkyl group is a C₁₋₄ haloalkyl group.

As used herein, the term “heteroalkyl,” by itself or in combination with another term, means, unless otherwise stated, a stable straight or branched-chain alkyl group, e.g., having from 2 to 14 carbons, such as 2 to 10 carbons in the chain, one or more of the carbons has been replaced by a heteroatom selected from S, O, P and N, and wherein the nitrogen, phosphine, and sulfur atoms can optionally be oxidized and the nitrogen heteroatom can optionally be quaternized. The heteroatom(s) S, O, P and N may be placed at any interior position of the heteroalkyl group or at the position at which the alkyl group is attached to the remainder of the molecule. When the heteroalkyl is said to be substituted, the substituent(s) can replace one or more hydrogen atoms attached to the carbon atom(s) and/or the heteroatom(s) of the heteroalkyl. In some embodiments, the heteroalkyl is a C₁₋₄ heteroalkyl, which refers to the heteroalkyl defined herein having 1-4 carbon atoms. Examples of C₁₋₄ heteroalkyl include, but are not limited to, C₄ heteroalkyl such as —CH₂—CH₂—N(CH₃)—CH₃, C₃ heteroalkyl such as —CH₂—CH₂—O—CH₃, —CH₂—CH₂—NH—CH₃, —CH₂—S—CH₂—CH₃, —CH₂—CH₂—S(O)—CH₃, —CH₂—CH₂—S(O)₂—CH₃, C₂ heteroalkyl such as —CH₂—CH₂—OH, —CH₂—CH₂—NH₂, —CH₂—NH(CH₃), —O—CH₂—CH₃ and C₁ heteroalkyl such as, —CH₂—OH, —CH₂—NH₂, —O—CH₃. Similarly, the term “heteroalkylene” by itself or as part of another substituent means a divalent radical derived from heteroalkyl, as exemplified, but not limited by, —CH₂—CH₂—O—CH₂—CH₂— and —O—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

“Carbocyclyl” or “carbocyclic” as used by itself or as part of another group refers to a radical of a non-aromatic cyclic hydrocarbon group having at least 3 carbon atoms, e.g., from 3 to 10 ring carbon atoms (“C₃₋₁₀ carbocyclyl”), and zero heteroatoms in the non-aromatic ring system. The carbocyclyl group can be either monocyclic (“monocyclic carbocyclyl”) or contain a fused, bridged or spiro ring system such as a bicyclic system (“bicyclic carbocyclyl”) and can be saturated or can be partially unsaturated. Non-limiting exemplary carbocyclyl groups include cyclopropyl, cyclobutyl, cyclopentyl, cyclohexyl, cycloheptyl, cyclooctyl, norbornyl, decalin, adamantyl, cyclopentenyl, and cyclohexenyl. As used herein, the term “carbocyclylene” as used by itself or as part of another group refers to a divalent radical derived from the carbocyclyl group defined herein.

In some embodiments, “carbocyclyl” is fully saturated, which is also referred to as cycloalkyl. In some embodiments, the cycloalkyl can have from 3 to 10 ring carbon atoms (“C₃₋₁₀ cycloalkyl”). In preferred embodiments, the cycloalkyl is a monocyclic ring. As used herein, the term “cycloalkylene” as used by itself or as part of another group refers to a divalent radical derived from a cycloalkyl group, for example,

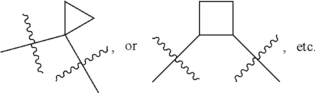

“Heterocyclyl” or “heterocyclic” as used by itself or as part of another group refers to a radical of a 3-membered or larger, such as 3- to 14-membered, non-aromatic ring system having ring carbon atoms and at least one ring heteroatom, such as 1 to 4 ring heteroatoms, wherein each heteroatom is independently selected from nitrogen, oxygen, sulfur, boron, phosphorus, and silicon. In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged, or spiro ring system, such as a bicyclic system (“bicyclic heterocyclyl”), and can be saturated or can be partially unsaturated. Heterocyclyl bicyclic ring systems can include one or more heteroatoms in one or both rings, and the point of attachment can be on any ring. As used herein, the term “heterocyclylene” as used by itself or as part of another group refers to a divalent radical derived from the heterocyclyl group defined herein. The heterocyclyl or heterocylylene can be optionally linked to the rest of the molecule through a carbon or nitrogen atom.

Exemplary 3-membered heterocyclyl groups containing one heteroatom include, without limitation, azirdinyl, oxiranyl, thiiranyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, and dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Aryl” as used by itself or as part of another group refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). As used herein, the term “arylene” as used by itself or as part of another group refers to a divalent radical derived from the aryl group defined herein.

“Aralkyl” as used by itself or as part of another group refers to an alkyl substituted with one or more aryl groups, preferably, substituted with one aryl group. Examples of aralkyl include benzyl, phenethyl, etc. When an aralkyl is said to be optionally substituted, either the alkyl portion or the aryl portion of the aralkyl can be optionally substituted.

“Heteroaryl” as used by itself or as part of another group refers to a radical of a 5-14 membered monocyclic, bicyclic, or tricyclic 4n+2 aromatic ring system (e.g., having 6 or 10 pi electrons shared in a cyclic array) having ring carbon atoms and at least one, preferably, 1-4, ring heteroatoms provided in the aromatic ring system, wherein each heteroatom is independently selected from nitrogen, oxygen and sulfur (“5-14 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. In bicyclic heteroaryl groups wherein one ring does not contain a heteroatom (e.g., indolyl, quinolinyl, and the like), the point of attachment can be on either ring, i.e., either the ring bearing a heteroatom (e.g., 2-indolyl) or the ring that does not contain a heteroatom (e.g., 5-indolyl). As used herein, the term “heteroarylene” as used by itself or as part of another group refers to a divalent radical derived from the heteroaryl group defined herein.

Exemplary 5-membered heteroaryl groups containing one heteroatom include, without limitation, pyrrolyl, furanyl, and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

“Heteroaralkyl” as used by itself or as part of another group refers to an alkyl substituted with one or more heteroaryl groups, preferably, substituted with one heteroaryl group. When a heteroaralkyl is said to be optionally substituted, either the alkyl portion or the heteroaryl portion of the heteroaralkyl can be optionally substituted.

An “optionally substituted” group, such as an optionally substituted alkyl, optionally substituted alkenyl, optionally substituted alkynyl, optionally substituted carbocyclyl, optionally substituted heterocyclyl, optionally substituted aryl, and optionally substituted heteroaryl groups, refers to the respective group that is unsubstituted or substituted. In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent can be the same or different at each position. Typically, when substituted, the optionally substituted groups herein can be substituted with 1-5 substituents. Substituents can be a carbon atom substituent, a nitrogen atom substituent, an oxygen atom substituent or a sulfur atom substituent, as applicable, each of which can be optionally isotopically labeled, such as deuterated. Two of the optional substituents can join to form a ring structure, such as an optionally substituted cycloalkyl, heterocylyl, aryl, or heteroaryl ring. Substitution can occur on any available carbon, oxygen, or nitrogen atom, and can form a spirocycle. Typically, substitution herein does not result in an O—O, O—N, S—S, S—N(except SO₂—N bond), heteroatom-halogen, or —C(O)—S bond or three or more consecutive heteroatoms, with the exception of O—SO₂—O, O—SO₂—N, and N—SO₂—N, except that some of such bonds or connections may be allowed if in a stable aromatic system.

In a broad aspect, the permissible substituents herein include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a cycloalkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, an aryl, or a heteroaryl, each of which can be substituted, if appropriate.

Exemplary substituents include, but not limited to, alkyl, alkenyl, alkynyl, aryl, heteroaryl, -alkylene-aryl, -arylene-alkyl, -alkylene-heteroaryl, -alkenylene-heteroaryl, -alkynylene-heteroaryl, OH, hydroxyalkyl, haloalkyl, O-alkyl, O-haloalkyl, -alkylene-O-alkyl, O-aryl, O-alkylene-aryl, acyl, C(O)-aryl, halo, NO₂, CN, SFs, C(O)OH, C(O)O-alkyl, C(O)O-aryl, C(O)O-alkylene-aryl, S(O)-alkyl, S(O)₂-alkyl, S(O)-aryl, S(O)₂-aryl, S(O)-heteroaryl, S(O)₂-heteroaryl, S-alkyl, S-aryl, S-heteroaryl, S-alkylene-aryl, S-alkylene-heteroaryl, S(O)₂-alkylene-aryl, S(O)₂-alkylene-heteroaryl, cycloalkyl, heterocycloalkyl, O—C(O)-alkyl, O—C(O)-aryl, —O—C(O)-cycloalkyl, C(═N—CN)—NH₂, C(═NH)—NH₂, C(═NH)—NH(alkyl), N(Y₁)(Y₂), -alkylene-N(Y₁)(Y₂), C(O)N(Y₁)(Y₂) and S(O)₂N(Y₁)(Y₂), wherein Y₁ and Y₂ can be the same or different and are independently selected from the group consisting of hydrogen, alkyl, aryl, cycloalkyl, and -alkylene-aryl.

Some examples of suitable substituents include, but not limited to, (C₁-C₈)alkyl groups, (C₂-C₈)alkenyl groups, (C₂-C₈)alkynyl groups, (C₃-C₁₀)cycloalkyl groups, halogen (F, Cl, Br or I), halogenated (C₁-C₈)alkyl groups (for example but not limited to CF₃), O—(C₁-C₈)alkyl groups, OH, S—(C₁-C₈)alkyl groups, SH, —NH(C₁-C₈)alkyl groups, N((C₁-C₈)alkyl)₂ groups, —NH₂, C(O)NH₂, C(O)NH(C₁-C₈)alkyl groups, C(O)N((C₁-C₈)alkyl)₂, —NHC(O)H, —NHC(O) (C₁-C₈)alkyl groups, —NHC(O) (C₃-C₈)cycloalkyl groups, N((C₁-C₈)alkyl)C(O)H, N((C₁-C₈)alkyl)C(O)(C₁-C₈)alkyl groups, NHC(O)NH₂, NHC(O)NH(C₁-C₈)alkyl groups, N((C₁-C₈)alkyl)C(O)NH₂ groups, NHC(O)N((C₁-C₈)alkyl)₂ groups, N((C₁-C₈)alkyl)C(O)N((C₁-C₈)alkyl)₂ groups, N((C₁-C₈)alkyl)C(O)NH((C₁-C₈)alkyl), C(O)H, C(O)(C₁-C₈)alkyl groups, CN, NO₂, S(O)(C₁-C₈)alkyl groups, S(O)₂(C₁-C₈)alkyl groups, S(O)₂N((C₁-C₈)alkyl)₂ groups, S(O)₂NH(C₁-C₈)alkyl groups, S(O)₂NH(C₃-C₈)cycloalkyl groups, S(O)₂NH₂ groups, NHS(O)₂(C₁-C₈)alkyl groups, N((C₁-C₈)alkyl)S(O)₂(C₁-C₈)alkyl groups, (C₁-C₈)alkyl-0 (C₁-C₈)alkyl groups, 0 (C₁-C₈)alkyl-O (C₁-C₈)alkyl groups, C(O)OH, C(O)O(C₁-C₈)alkyl groups, NHOH, NHO(C₁-C₈)alkyl groups, O-halogenated (C₁-C₈)alkyl groups (for example but not limited to OCF3), S(O)₂-halogenated (C₁-C₈)alkyl groups (for example but not limited to S(O)₂CF₃), —S-halogenated (C₁-C₈)alkyl groups (for example but not limited to SCF3), (C₁-C₆) heterocycle (for example but not limited to pyrrolidine, tetrahydrofuran, pyran or morpholine), (C₁-C₆) heteroaryl (for example but not limited to tetrazole, imidazole, furan, pyrazine or pyrazole), -phenyl, —NHC(O)O—(C₁-C₆)alkyl groups, N((C₁-C₆)alkyl)C(O)O (C₁-C₆)alkyl groups, C(═NH)—(C₁-C₆)alkyl groups, C(═NOH)—(C₁-C₆)alkyl groups, or C(═N—O—(C₁-C₆)alkyl)-(C₁-C₆)alkyl groups.

Exemplary carbon atom substituents include, but are not limited to, deuterium, halogen, —CN, —NO₂, —N₃, hydroxyl, alkoxy, cycloalkoxy, aryloxy, amino, monoalkyl amino, dialkyl amino, amide, sulfonamide, thiol, acyl, carboxylic acid, ester, sulfone, sulfoxide, alkyl, haloalkyl, alkenyl, alkynyl, C₃₋₁₀ carbocyclyl, C₆₋₁₀ aryl, 3-10 membered heterocyclyl, 5-10 membered heteroaryl, etc. For example, exemplary carbon atom substituents can include F, Cl, —CN, —SO₂H, —SO₃H, —OH, —OC₁₋₆ alkyl, —NH₂, —N(C₁₋₆ alkyl)₂, —NH(C₁₋₆ alkyl), —SH, —SC₁₋₆ alkyl, —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, C₁₋₆ alkyl, C₁₋₆ haloalkyl, C₂₋₆ alkenyl, C₂₋₆ alkynyl, C₃₋₁₀ carbocyclyl, C₆₋₁₀ aryl, 3-10 membered heterocyclyl, 5-10 membered heteroaryl; or two geminal substituents can be joined to form ═O.

Nitrogen atoms can be substituted or unsubstituted as valency permits, and include primary, secondary, tertiary, and quaternary nitrogen atoms. Exemplary nitrogen atom substituents include, but are not limited to, hydrogen, acyl groups, esters, sulfone, sulfoxide, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₄ aryl, and 5-14 membered heteroaryl, or two substituent groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl can be further substituted as defined herein. In certain embodiments, the substituent present on a nitrogen atom is a nitrogen protecting group (also referred to as an amino protecting group). Nitrogen protecting groups are well known in the art and include those described in detail in Protective Groups in Organic Synthesis, T. W. Greene and P. G. M. Wuts, 3^{rd} edition, John Wiley & Sons, 1999, incorporated by reference herein. Exemplary nitrogen protecting groups include, but not limited to, those forming carbamates, such as Carbobenzyloxy (Cbz) group, p-Methoxybenzyl carbonyl (Moz or MeOZ) group, tert-Butyloxycarbonyl (BOC) group, Troc, 9-Fluorenylmethyloxycarbonyl (Fmoc) group, etc., those forming an amide, such as acetyl, benzoyl, etc., those forming a benzylic amine, such as benzyl, p-methoxybenzyl, 3,4-dimethoxybenzyl, etc., those forming a sulfonamide, such as tosyl, Nosyl, etc., and others such as p-methoxyphenyl.

Exemplary oxygen atom substituents include, but are not limited to, acyl groups, esters, sulfonates, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl can be further substituted as defined herein. In certain embodiments, the oxygen atom substituent present on an oxygen atom is an oxygen protecting group (also referred to as a hydroxyl protecting group). Oxygen protecting groups are well known in the art and include those described in detail in Protective Groups in Organic Synthesis, T. W. Greene and P. G. M. Wuts, 3^{rd} edition, John Wiley & Sons, 1999, incorporated herein by reference. Exemplary oxygen protecting groups include, but are not limited to, those forming alkyl ethers or substituted alkyl ethers, such as methyl, allyl, benzyl, substituted benzyls such as 4-methoxybenzyl, methoxylmethyl (MOM), benzyloxymethyl (BOM), 2-methoxyethoxymethyl (MEM), etc., those forming silyl ethers, such as trymethylsilyl (TMS), triethylsilyl (TES), triisopropylsilyl (TIPS), t-butyldimethylsilyl (TBDMS), etc., those forming acetals or ketals, such as tetrahydropyranyl (THP), those forming esters such as formate, acetate, chloroacetate, dichloroacetate, trichloroacetate, trifluoroacetate, methoxyacetate, etc., those forming carbonates or sulfonates such as methanesulfonate (mesylate), benzylsulfonate, and tosylate (Ts), etc.

Unless expressly stated to the contrary, combinations of substituents and/or variables are allowable only if such combinations are chemically allowed and result in a stable compound. A “stable” compound is a compound that can be prepared and isolated and whose structure and properties remain or can be caused to remain essentially unchanged for a period of time sufficient to allow use of the compound for the purposes described herein (e.g., therapeutic administration to a subject).

In some embodiments, the “optionally substituted” alkyl, alkylene, heteroalkyl, heteroalkylene, alkenyl, alkynyl, carbocyclic, carbocyclylene, cycloalkyl, cycloalkylene, alkoxy, cycloalkoxy, heterocyclyl, or heterocyclylene herein can each be independently unsubstituted or substituted with 1, 2, 3, or 4 substituents independently selected from deuterium, F, Cl, —OH, protected hydroxyl, oxo (as applicable), NH₂, protected amino, NH(C₁₋₄ alkyl) or a protected derivative thereof, N(C₁₋₄ alkyl((C₁₋₄ alkyl), C₁₋₄ alkyl, C₂₋₄ alkenyl, C₂₋₄ alkynyl, C₁₋₄ alkoxy, C₃₋₆ cycloalkyl, C₃₋₆ cycloalkoxy, phenyl, 5 or 6 membered heteroaryl containing 1, 2, or 3 ring heteroatoms independently selected from O, S, and N, 3-7 membered heterocyclyl containing 1 or 2 ring heteroatoms independently selected from O, S, and N, wherein each of the alkyl, alkenyl, alkynyl, alkoxy, cycloalkyl, cycloalkoxy phenyl, heteroaryl, and heterocyclyl, is optionally substituted with 1, 2, or 3 substituents independently selected from deuterium, F, —OH, oxo (as applicable), C₁₋₄ alkyl, fluoro-substituted C₁₋₄ alkyl (e.g., CF₃), C₁₋₄ alkoxy and fluoro-substituted C₁₋₄ alkoxy. In some embodiments, the “optionally substituted” aryl, arylene, heteroaryl or heteroarylene group herein can each be independently unsubstituted or substituted with 1, 2, 3, or 4 substituents independently selected from deuterium, F, Cl, —OH, —CN, NH₂, protected amino, NH(C₁₋₄ alkyl) or a protected derivative thereof, N(C₁₋₄ alkyl((C₁₋₄ alkyl), —S(═O)(C₁₋₄ alkyl), —SO₂(C₁₋₄ alkyl), C₁₋₄ alkyl, C₂₋₄ alkenyl, C₂₋₄ alkynyl, C₁₋₄ alkoxy, C₃₋₆ cycloalkyl, C₃₋₆ cycloalkoxy, phenyl, 5 or 6 membered heteroaryl containing 1, 2 or 3 ring heteroatoms independently selected from O, S, and N, 3-7 membered heterocyclyl containing 1 or 2 ring heteroatoms independently selected from O, S, and N, wherein each of the alkyl, alkenyl, alkynyl, alkoxy, cycloalkyl, cycloalkoxy, phenyl, heteroaryl, and heterocyclyl, is optionally substituted with 1, 2, or 3 substituents independently selected from deuterium, F, —OH, oxo (as applicable), C₁₋₄ alkyl, fluoro-substituted C₁₋₄ alkyl, C₁₋₄ alkoxy and fluoro-substituted C₁₋₄ alkoxy.

“Halo” or “halogen” refers to fluorine (fluoro, —F), chlorine (chloro, —Cl), bromine (bromo, —Br), or iodine (iodo, —I).

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art.

The term “tautomers” or “tautomeric” refers to two or more interconvertible compounds resulting from tautomerization. The exact ratio of the tautomers depends on several factors, including for example temperature, solvent, and pH. Tautomerizations are known to those skilled in the art. Exemplary tautomerizations include keto-to-enol, amide-to-imide, lactam-to-lactim, enamine-to-imine, and enamine-to-(a different enamine) tautomerizations.

The term “subject” (alternatively referred to herein as “patient”) as used herein, refers to an animal, preferably a mammal, most preferably a human, who has been the object of treatment, observation or experiment.

As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated. As used herein, the terms “treat,” “treating,” “treatment,” and the like may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition. The term “treat” and synonyms contemplate administering a therapeutically effective amount of a compound described herein to a subject in need of such treatment.

The term “effective amount” refers to that amount of a compound or combination of compounds as described herein that is sufficient to effect the intended application including, but not limited to, prophylaxis or treatment of diseases. A therapeutically effective amount may vary depending upon the intended application (in vitro or in vivo), or the subject and disease condition being treated (e.g., the weight, age and gender of the subject), the severity of the disease condition, the manner of administration, etc. which can readily be determined by one of ordinary skill in the art. The term also applies to a dose that will induce a particular response in target cells and/or tissues. The specific dose will vary depending on the particular compounds chosen, the dosing regimen to be followed, whether the compound is administered in combination with other compounds, timing of administration, the tissue to which it is administered, and the physical delivery system in which the compound is carried.

As used herein, the singular form “a”, “an”, and “the”, includes plural references unless it is expressly stated or is unambiguously clear from the context that such is not intended.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Headings and subheadings are used for convenience and/or formal compliance only, do not limit the subject technology, and are not referred to in connection with the interpretation of the description of the subject technology. Features described under one heading or one subheading of the subject disclosure may be combined, in various embodiments, with features described under other headings or subheadings. Further it is not necessarily the case that all features under a single heading or a single subheading are used together in embodiments.

### EXAMPLES

The various starting materials, intermediates, and compounds of embodiments herein can be isolated and purified where appropriate using conventional techniques such as precipitation, filtration, crystallization, evaporation, distillation, and chromatography. Characterization of these compounds can be performed using conventional methods such as by melting point, mass spectrum, nuclear magnetic resonance, and various other spectroscopic analyses. The abbreviations used in the Examples section should be understood as having their ordinary meanings in the art unless specifically indicated otherwise or obviously contrary from context. The examples are illustrative only and do not limit the claimed invention in any way.

Exemplary embodiments of steps for performing the synthesis of products described herein are described in greater detail infra. Some of the Examples discussed herein can be prepared by separating from the corresponding racemic mixtures. As would be understood by a person of ordinary skill in the art, the compounds described in the Examples section immediately prior to the chiral separation step, e.g., by supercritical fluid chromatography (SFC), exist in racemic and/or stereoisomeric mixture forms, the bolded but not wedged bonds are used in the chemical structure drawings to indicate relative stereochemistry. It should be understood that the enantiomeric excesses (“ee”) reported for these examples are only representative from the exemplified procedures herein and not limiting; those skilled in the art would understand that such enantiomers with a different ee, such as a higher ee, can be obtained in view of the present disclosure.

In some illustrative examples, the synthesis of a deuterated compound is shown. To the extent applicable, it should be understood that the corresponding non-deuterated (i.e., with natural abundance) compound was prepared through the same method except by using a corresponding non-deuterated starting material or intermediate.

### Synthesis of cis-1-methylcyclopentane-1,2-diol (Intermediate I)

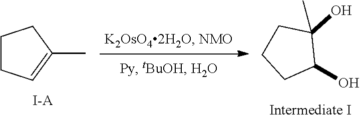

To a solution of 1-methylcyclopent-1-ene (I-A, 9.20 g, 112 mmol) in t-BuOH (90 mL) and H₂O (30 mL) were added potassium dioxidodioxoosmium dihydrate (2.06 g, 5.60 mmol), 4-methylmorpholine N-oxide (NMO) (18.3 g, 157 mmol) and pyridine (9.0 mL, 112 mmol). The reaction mixture was stirred at 85° C. for 5 hours. After completion, the mixture was filtered through a short pad of Celite©, and the filtrate was quenched with saturated NaHSO₃ solution (20 mL), concentrated under reduced pressure to yield a residue, which was separated using silica gel column chromatography to afford cis-1-methylcyclopentane-1,2-diol (Intermediate I, 11.9 g, 91%) as an oil. ¹H NMR (400 MHz, DMSO-d₆) δ 4.36 (d, J=5.5 Hz, 1H), 3.83 (s, 1H), 3.48-3.34 (m, 1H), 1.81-1.33 (m, 6H), 1.09 (s, 3H).

### Synthesis of cis-4,4-difluoro-1-methylcyclopentane-1,2-diol (Intermediate II)

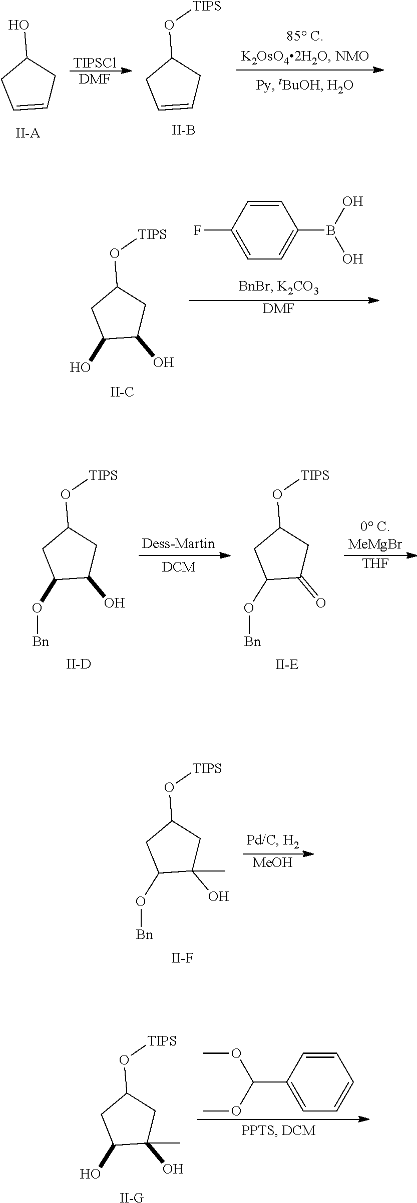

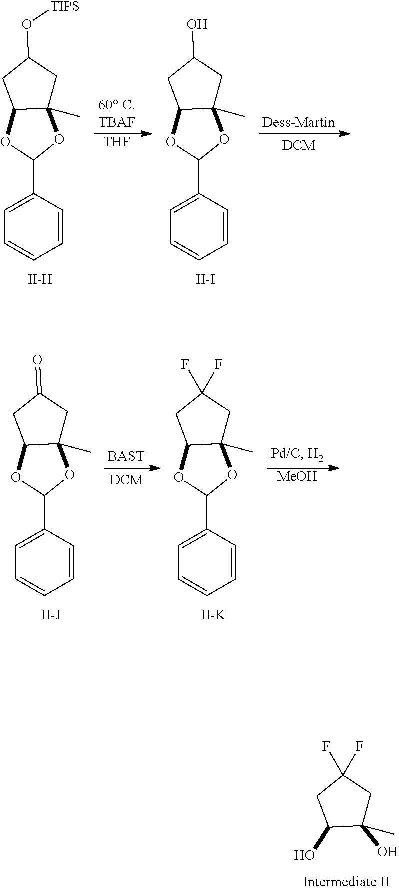

To a mixture of cyclopent-3-en-1-ol (II-A, 5.00 g, 59.4 mmol) and 1H-imidazole (4.45 g, 65.4 mmol) in DMF (50 mL) was added dropwise chlorotriisopropylsilane (11.5 g, 59.4 mmol). The mixture was stirred at room temperature for 12 hours. The resulting mixture was diluted with water (100 mL) and extracted with n-hexane (50 mL×3). The combined organic layers were washed with brine (50 mL), dried over sodium sulphate and concentrated under reduced pressure to give cyclopent-3-en-1-yloxy)triisopropylsilane (II-B, 14.5 g, crude) as a yellow oil. 1H NMR (500 MHz, CDCl₃) δ 5.68 (s, 2H), 4.67-4.62 (m, 1H), 2.65 (dd, J=7.0, 1.9 Hz, 1H), 2.61 (dd, J=7.0, 1.7 Hz, 1H), 2.37 (t, J=2.7 Hz, 1H), 2.33 (t, J=2.9 Hz, 1H), 1.13-1.08 (m, 21H).

To a solution of (cyclopent-3-en-1-yloxy)tris(propan-2-yl)silane (II-B, 5.00 g, crude product from above) in t-BuOH (50 mL) were added potassium osmate (0.38 g, 1.04 mmol), 4-methylmorpholine N-oxide (NMO) (3.41 g, 29.1 mmol), pyridine (1.67 mL, 20.8 mmol) and water (15 mL). The reaction mixture was stirred at 85° C. for 5 hours. The resulting mixture was concentrated to give a residue, which was subjected to silica gel column chromatography eluted with petroleum ether/ethyl acetate (from 10:1 to 1:1) to give cis-4-((triisopropylsilyl)oxy)cyclopentane-1,2-diol (I-C, 3.2 g) as a yellow oil. ¹H NMR (500 MHz, CDCl₃) δ 4.54-4.50 (m, 1H), 4.28 (t, J=4.9 Hz, 2H), 2.73 (s, 2H), 2.04-1.99 (m, 2H), 1.95-1.90 (m, 2H), 1.08-1.03 (m, 21H).

To a mixture of cis-4-((triisopropylsilyl)oxy)cyclopentane-1,2-diol (II-C, 2.20 g, 8.01 mmol) and (4-fluorophenyl)boronic acid (0.11 g, 0.80 mmol) in N,N-dimethylformamide (20 mL) were added potassium carbonate (1.66 g, 12.0 mmol) and (bromomethyl)benzene (2.06 g, 12.0 mmol). The mixture was stirred at room temperature for 12 hours under N₂ atmosphere. The resulting mixture was diluted with water (100 mL) and extracted with ethyl acetate (50 mL×3). The combined organic layers were washed with brine (50 mL), dried over sodium sulphate and concentrated under reduced pressure to give a residue, which was subjected to silica gel column chromatography eluted with petroleum ether/ethyl acetate (from 1:0 to 10:1) to give cis-2-(benzyloxy)-4-((triisopropylsilyl)oxy)cyclopentan-1-ol (II-D, 1.70 g, 58%) as a yellow oil. ¹H NMR (500 MHz, CDCl₃) δ 7.41-7.30 (m, 5H), 4.63 (d, J=11.7 Hz, 1H), 4.60-4.52 (m, 2H), 4.31 (q, J=4.7 Hz, 1H), 4.13-4.10 (m, 1H), 2.16-2.07 (m, 2H), 1.94-1.90 (m, 1H), 1.86-1.81 (m, 1H), 1.08-1.40 (m, 21H).

To a solution of cis-2-(benzyloxy)-4-((triisopropylsilyl)oxy)cyclopentan-1-ol (II-D, 1.20 g, 3.29 mmol) in dichloromethane (20 mL) was added Dess-Martin Periodinane (2.79 g, 6.58 mmol) at 0° C., and the resulting mixture was stirred at room temperature for 5 hours. After completion, the reaction mixture was quenched with saturated sodium thiosulfate solution (30 mL), diluted with water (50 mL) and then extracted with dichloromethane (50 mL×3). The combined organic layers were washed with brine (50 mL), dried over sodium sulphate and concentrated under reduced pressure to give a residue, which was subjected to silica gel column chromatography eluted with petroleum ether/ethyl acetate (from 1:0 to 10:1) to give 2-(benzyloxy)-4-((triisopropylsilyl)oxy) cyclopentan-1-one (II-E, 1.00 g, 84%) as a yellow oil. ¹H NMR (500 MHz, CDCl₃) δ 7.48-7.29 (m, 5H), 4.91 (d, J=11.7 Hz, 1H), 4.77-4.57 (m, 2H), 4.21 (t, J=8.6 Hz, 1H), 2.57-2.52 (m, 1H), 2.39-2.33 (m, 2H), 2.06-2.00 (m, 1H), 1.10-0.97 (m, 21H).

To a solution of 2-(benzyloxy)-4-{[tris(propan-2-yl)silyl]oxy}cyclopentan-1-one (II-E, 2.30 g, 6.34 mmol) in dry THE (20 mL) was added dropwise methylmagnesium bromide (1 M in THF, 12.7 mL, 12.7 mmol) at 0° C. The mixture was then stirred at room temperature for 1 hour. After completion, the resulting mixture was diluted with saturated ammonium chloride solution (10 mL) and water (20 mL), and then extracted with ethyl acetate (20 mL×3). The combined organic layers were washed with brine (20 mL), dried over sodium sulphate and concentrated under reduced pressure to give a residue. The residue was subjected to silica gel column chromatography eluted with petroleum ether/ethyl acetate (from 1:0 to 1:1) to give 2-(benzyloxy)-1-methyl-4-((triisopropylsilyl)oxy)cyclopentan-1-ol (II-F, 1.50 g, 63%) as a yellow oil.

A mixture of 2-(benzyloxy)-1-methyl-4-((triisopropylsilyl)oxy)cyclopentan-1-ol (II-F, 1.20 g, 3.17 mmol) and palladium (10% on carbon, 0.2 g) in methanol (12 mL) was stirred at room temperature under one atmosphere of H₂. The resulting mixture was filtered, and the filtrate was concentrated to give a residue which was subjected to silica gel column chromatography eluted with petroleum ether/ethyl acetate (from 10:1 to 0:1) to give cis-1-methyl-4-((triisopropylsilyl)oxy)cyclopentane-1,2-diol (II-G, 650 mg, 71%) as a yellow oil. ¹H NMR (500 MHz, CDCl₃) δ 4.59-4.53 (m, 1H), 4.15-4.13 (m, 1H), 3.85 (s, 1H), 2.41-2.36 (m, 1H), 1.98-1.95 (m, 2H), 1.87-1.75 (m, 2H), 1.32 (s, 3H), 1.08-1.06 (m, 21H).

A mixture of cis-1-methyl-4-((triisopropylsilyl)oxy)cyclopentane-1,2-diol (II-G, 500 mg, 1.73 mmol), (dimethoxymethyl)benzene (395 mg, 2.60 mmol) and pyridinium p-toluenesulfonate (PPTS) (10 mg, 0.055 mmol) in dichloromethane (3 mL) was stirred at room temperature for 4 hours. The resulting mixture was concentrated to give a residue, which was subjected to silica gel column chromatography eluted with petroleum ether/ethyl acetate (from 100:1 to 2:1) to give cis-triisopropyl((3a-methyl-2-phenyltetrahydro-4H-cyclopenta[d][1,3]dioxol-5-yl)oxy)silane (II-H, 350 mg, 54%) as a yellow oil. ¹H NMR (500 MHz, CDCl₃) δ 7.50-7.48 (m, 2H), 7.42-7.37 (m, 3H), 5.71 (s, 1H), 4.77-4.71 (m, 1H), 4.27 (d, J=5.6 Hz, 1H), 2.43-2.39 (m, 1H), 2.35-2.30 (m, 1H), 1.77-7.71 (m, J=1H), 1.57-1.54 (m, 1H), 1.52 (s, 3H), 1.09-1.08 (m, 21H).

A mixture of cis-triisopropyl((3a-methyl-2-phenyltetrahydro-4H-cyclopenta[d][1,3]dioxol-5-yl)oxy)silane (II-H, 350 mg, 0.93 mmol) and tetrabutylammonium fluoride (1 M in THF, 5 mL) was stirred at 60° C. for 1 hour. The mixture was concentrated under reduced pressure to give a residue, which was subjected to silica gel column chromatography eluted with petroleum ether/ethyl acetate (from 10:1 to 1:1) to give cis-3a-methyl-2-phenyltetrahydro-4H-cyclopenta[d][1,3]dioxol-5-ol (II-I, 170 mg, 85%) as a yellow oil. ¹H NMR (500 MHz, CDCl₃) δ 7.50 (dd, J=6.5, 3.0 Hz, 2H), 7.41-7.39 (m, 3H), 5.72 (s, 1H), 4.75-4.69 (m, 1H), 4.30 (d, J=5.7 Hz, 1H), 2.49-2.45 (m, 1H), 2.41-2.37 (m, 1H), 1.75-1.69 (m, 1H), 1.54 (s, 3H), 1.53-1.48 (m, 1H).

To a solution of cis-3a-methyl-2-phenyltetrahydro-4H-cyclopenta[d][1,3]dioxol-5-01 (II-I, 150 mg, 0.68 mmol) in dichloromethane (4 mL) was added Dess-Martin Periodinane (347 mg, 0.80 mmol) at 0° C. The mixture was stirred at room temperature for 12 hours. The resulting mixture was filtered. The filter cake was washed with ethyl acetate (20 mL). The filtrate was concentrated to give a residue, which was subjected to silica gel column chromatography eluted with petroleum ether/ethyl acetate (from 20:1 to 3:1) to give cis-3a-methyl-2-phenyltetrahydro-5H-cyclopenta[d][1,3]dioxol-5-one (II-J, 120 mg, 80%) as a yellow oil. ¹H NMR (500 MHz, CDCl₃) δ 7.49-7.44 (m, 2H), 7.41-7.39 (m, 3H), 5.96 (s, 1H), 4.55 (dd, J=5.0, 3.2 Hz, 1H), 2.83-2.79 (m, 1H), 2.75-2.70 (m, 2H), 2.50-2.47 (m, 1H), 1.64 (s, 3H).

A mixture of cis-3a-methyl-2-phenyltetrahydro-5H-cyclopenta[d][1,3]dioxol-5-one (II-J, 380 mg, 1.74 mmol) and bis(2-methoxyethyl)aminosulfur trifluoride (BAST) (0.96 mL, 5.22 mmol) in dichloromethane (2 mL) was stirred at room temperature for 48 hours. The resulting mixture was diluted with water (10 mL) and extracted with ethyl acetate (10 mL×3). The combined organic layers were washed with brine (20 mL), dried over sodium sulphate and concentrated under reduced pressure to give a residue, which was subjected to silica gel column chromatography eluted with petroleum ether/ethyl acetate (from 50:1 to 1:1) to give cis-5,5-difluoro-3a-methyl-2-phenyltetrahydro-4H-cyclopenta[d][1,3]dioxole (II-K, 290 mg, 69%) as a yellow oil. ¹H NMR (500 MHz, CDCl₃) δ 7.59-7.54 (m, 2H), 7.46-7.34 (m, 3H), 5.80 (s, 1H), 4.39 (d, J=6.6 Hz, 1H), 2.75-2.65 (m, 1H), 2.62-2.56 (m, 1H), 2.48-2.36 (m, 1H), 2.23-2.15 (m, 1H), 1.57 (s, 3H).

A mixture of cis-5,5-difluoro-3a-methyl-2-phenyltetrahydro-4H-cyclopenta[d][1,3]dioxole (II-K, 290 mg, 1.20 mmol), palladium (10% on carbon, 20 mg) and acetic acid (35 uL, 0.60 mmol) in methanol (10 mL) was stirred at room temperature for 12 hours under one atmosphere of H₂. The resulting mixture was filtered. The filtrate was concentrated to give a residue, which was subjected to silica gel column chromatography eluted with petroleum ether/ethyl acetate (from 10:1 to 0:1) to give cis-4,4-difluoro-1-methylcyclopentane-1,2-diol (Intermediate II, 160 mg, 89%) as a yellow oil. ¹H NMR (500 MHz, CDCl₃) δ 4.01-3.93 (m, 1H), 2.57-2.11 (m, 4H), 1.38 (s, 3H).

### Synthesis of cis-1-methylcyclopentane-4,4-d₂-1,2-diol (Intermediate III)

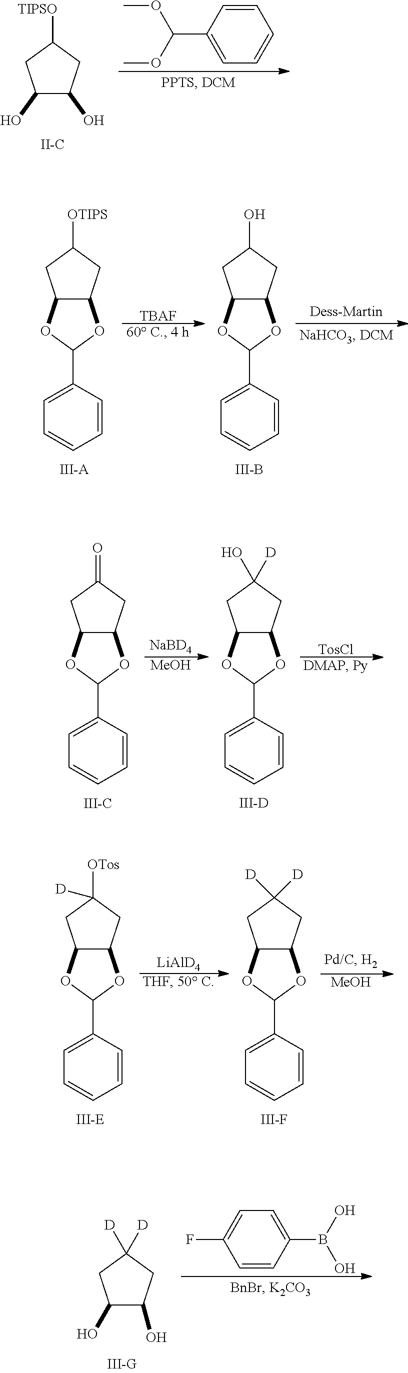

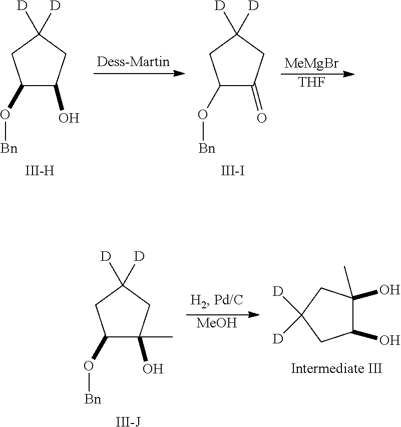

A mixture of cis-4-((triisopropylsilyl)oxy)cyclopentane-1,2-diol (II-C, 6.00 g, 21.9 mmol), (dimethoxymethyl)benzene (4.99 g, 32.8 mmol) and pyridinium p-toluenesulfonate (PPTS) (1.10 g, 4.37 mmol) in dichloromethane (60 mL) was stirred at room temperature for 4 hours. The mixture was concentrated under reduced pressure to give a residue, which was subjected to silica gel column chromatography eluted with petroleum ether/ethyl acetate (from 1:0 to 3:1) to give cis-triisopropyl((2-phenyltetrahydro-4H-cyclopenta[d][1,3]dioxol-5-yl)oxy)silane (III-A, 8.90 g, crude) as a yellow oil.

A mixture of cis-triisopropyl((2-phenyltetrahydro-4H-cyclopenta[d][1,3]dioxol-5-yl)oxy)silane (III-A, 8.00 g, crude from above) and tetrabutylammonium fluoride (TBAF) (1 M in THF, 50.0 mL, 50.0 mmol) was stirred at 60° C. for 1 hour. The reaction mixture was concentrated under reduced pressure to give a residue, which was subjected to silica gel column chromatography eluted with petroleum ether/ethyl acetate (from 10:1 to 1:1) to give cis-2-phenyltetrahydro-4H-cyclopenta[d][1,3]dioxol-5-ol (III-B, 350 mg) as a yellow oil. ¹H NMR (500 MHz, CDCl₃) δ 7.50-7.48 (m, 2H), 7.42-7.40 (m, 3H), 5.63 (s, 1H), 4.72 (dd, J=4.1, 1.9 Hz, 2H), 4.70-4.63 (m, 1H), 2.39-2.35 (m, 2H), 1.67-1.62 (m, 2H).

To a mixture of cis-2-phenyltetrahydro-4H-cyclopenta[d][1,3]dioxol-5-ol (III-B, 4.30 g, 20.9 mmol) and sodium bicarbonate (5.25 g, 62.6 mmol) in dichloromethane (40 mL) was added Dess-Martin Periodinane (10.6 g, 25.0 mmol) at 0° C. The mixture was stirred at room temperature for 12 hours. The reaction mixture was filtered. The filtrate was concentrated under reduced pressure to give a residue, which was subjected to silica gel column chromatography eluted with petroleum ether/ethyl acetate (from 20:1 to 3:1) to give cis-2-phenyltetrahydro-4H-cyclopenta[d][1,3]dioxol-5-ol (III-C, 3.10 g, 73%) as a yellow oil. ¹H NMR (500 MHz, CDCl₃) δ 7.49-7.46 (m, 2H), 7.44-7.38 (m, 3H), 5.88 (s, 1H), 4.96-4.94 (m, 2H), 2.67-2.63 (m, 4H).

To a solution of cis-2-phenyl-hexahydrocyclopenta[d][1,3]dioxol-5-one (III-C, 2.00 g, 9.79 mmol) in methanol (20 mL) was added sodium borodeuteride (1.91 g, 9.79 mmol) at 0° C. The mixture was stirred at room temperature for 4 hours. The reaction mixture was filtered and the filtrate concentrated under reduced pressure to give a residue. The residue was subjected to silica gel column chromatography eluted with petroleum ether/ethyl acetate (from 10:1 to 1:1) to give cis-2-phenyltetrahydro-4H-cyclopenta[d][1,3]dioxol-5-d-5-ol (III-D, 2 g, 98%) as a yellow oil. ¹H NMR (500 MHz, CDCl₃) δ 7.56-7.54 (m, 2H), 7.42-7.41 (m, 3H), 5.74 (s, 1H), 4.85-4.83 (m, 2H), 2.48-2.43 (m, 1H), 2.35-2.31 (m, 2H), 1.88-1.84 (m, 2H).

To a mixture of cis-2-phenyltetrahydro-4H-cyclopenta[d][1,3]dioxol-5-d-5-ol (III-D, 2.90 g, 15.0 mmol) and 4-dimethylaminopyridine (1.88 g, 15.4 mmol) in pyridine (30 mL) was added tosyl chloride (4.07 g, 26.6 mmol). The reaction mixture was stirred at room temperature for 24 hours. The reaction mixture was concentrated under reduced pressure to give a residue, which was subjected to silica gel column chromatography eluted with petroleum ether/ethyl acetate (from 10:1 to 1:1) to give cis-2-phenyltetrahydro-4H-cyclopenta[d][1,3]dioxol-5-yl-5-d 4-methylbenzenesulfonate (III-E, 4.10 g, 81%) as a yellow oil. ¹H NMR (500 MHz, CDCl₃) δ 7.81-7.71 (m, 2H), 7.59-7.52 (m, 2H), 7.40-7.36 (m, 1H), 7.33 (dd, J=8.1, 6.4 Hz, 2H), 7.30-7.27 (m, 2H), 5.67 (s, 1H), 4.75 (dd, J=4.4, 1.7 Hz, 2H), 2.44 (s, 3H), 2.43 (d, J=1.2 Hz, 1H), 2.40 (d, J=1.5 Hz, 1H), 1.96 (dd, J=4.4, 1.9 Hz, 1H), 1.92 (dd, J=4.5, 1.8 Hz, 1H).

To a solution of cis-2-phenyltetrahydro-4H-cyclopenta[d][1,3]dioxol-5-yl-5-d 4-methylbenzenesulfonate (III-E, 1.00 g, 2.77 mmol) in THE (10 mL) was added lithium aluminum deuteride (460 mg, 11.1 mmol) at 0° C. The mixture was stirred at 50° C. for 12 hours. To the reaction mixture were added sodium sulfate decahydrate until the bubbling ended, and then ethyl acetate (50 mL). The mixture was filtered, and the filtrate was concentrated to give a residue, which was subjected to silica gel column chromatography eluted with petroleum ether/ethyl acetate (from 1:0 to 3:1) to give cis-2-phenyltetrahydro-4H-cyclopenta[d][1,3]dioxole-5,5-d₂ (III-F, 443 mg, 83%) as a yellow oil. ¹H NMR (500 MHz, CDCl₃) δ 7.54-7.52 (m, 2H), 7.47-7.33 (m, 3H), 5.64 (s, 1H), 4.71-4.70 (m, 2H), 2.08-2.06 (m, 2H), 1.56-1.47 (m, 2H).

A mixture of cis-2-phenyltetrahydro-4H-cyclopenta[d][1,3]dioxole-5,5-d₂ (III-F, 440 mg, 1.22 mmol), palladium (10% on carbon, 500 mg) and acetic acid (770 uL, 1.35 mmol) in methanol (20 mL) was stirred under one atmosphere of H₂ at room temperature for 12 hours. The reaction mixture was filtered. The filtrate was concentrated under reduced pressure to give cis-cyclopentane-4,4-d₂-1,2-diol (III-G, 580 mg, 82%) as a yellow oil. ¹H NMR (500 MHz, CDCl₃) δ 4.08-4.05 (m, 2H), 1.92-1.85 (m, 2H), 1.73-1.63 (m, 2H).

To a mixture of cis-cyclopentane-4,4-d₂-1,2-diol (III-G, 190 mg, 1.82 mmol) and (4-fluorophenyl)boronic acid (25 mg, 0.20 mmol) in DMF (2 mL) were added K₂CO₃ (378 mg, 2.74 mmol) and (bromomethyl)benzene (468 mg, 2.74 mmol). The mixture was stirred at room temperature for 12 hours under N₂ atmosphere. The reaction mixture was diluted with water (50 mL) and then extracted with ethyl acetate (20 mL×3). The combined organic layers were washed with brine (10 mL), dried over sodium sulphate and concentrated to give a residue. The residue was subjected to silica gel column chromatography eluted with petroleum ether/ethyl acetate (from 1:0 to 3:1) to give cis-2-(benzyloxy)cyclopentan-4,4-d₂-1-ol (III-H, 270 mg, 77%) as a yellow oil. ¹H NMR (500 MHz, CDCl₃) δ 7.41-7.36 (m, 4H), 7.34-7.32 (m, 1H), 4.64 (d, J=11.8 Hz, 1H), 4.57 (d, J=11.8 Hz, 1H), 4.13-4.10 (m, 1H), 3.86-3.82 (m, 1H), 1.90-1.84 (m, 1H), 1.81-1.73 (m, 3H).

To a mixture of cis-2-(benzyloxy)cyclopentan-4,4-d₂-1-ol (III-H, 260 mg, 1.34 mmol) and sodium bicarbonate (337 mg, 4.02 mmol) in dichloromethane (5 mL) was added Dess-Martin Periodinane (681 mg, 1.61 mmol) at 0° C. The mixture was stirred at room temperature for 12 hours. The reaction was diluted with saturated aqueous sodium sulfite solution (20 mL) and water (20 mL). The mixture was extracted with dichloromethane (20 mL×3). The combined organic layers were washed with brine (20 mL), and dried over sodium sulphate and concentrated under reduced pressure to yield a residue. The residue was subjected to silica gel column chromatography eluted with petroleum ether/ethyl acetate (from 1:0 to 3:1) to give 2-(benzyloxy)cyclopentan-1-one-4,4-d₂ (III-I, 190 mg, 73%) as a yellow oil. ¹H NMR (500 MHz, CDCl₃) δ 7.42-7.34 (m, 4H), 7.35-7.29 (m, 1H), 4.86 (d, J=11.9 Hz, 1H), 4.71 (d, J=11.9 Hz, 1H), 3.81-3.84 (m, 1H), 2.33-2.21 (m, 3H), 1.87-1.83 (m, 1H).

To a solution of 2-(benzyloxy)cyclopentan-1-one-4,4-d₂ (III-I, 190 mg, 0.99 mmol) in tetrahydrofuran (4 mL) was added methylmagnesium bromide (0.66 mL, 1.98 mmol) dropwise at 0° C. The mixture was stirred at 0° C. for 2 hours. The reaction mixture was diluted with saturated aqueous ammonium chloride solution (10 mL) and water (20 mL). The mixture was extracted with ethyl acetate (20 mL×3). The combined organic layers were washed with brine (10 mL), dried over sodium sulphate, and then concentrated under reduced pressure to give a residue, which was subjected to silica gel column chromatography eluted with petroleum ether/ethyl acetate (from 10:1 to 1:1) to give cis-2-(benzyloxy)-1-methylcyclopentan-4,4-d₂-1-ol (III-J, 70 mg, 34%) as a yellow oil. ¹H NMR (500 MHz, CDCl₃) δ 7.41-7.34 (m, 4H), 7.35-7.30 (m, 1H), 4.68 (d, J=11.8 Hz, 1H), 4.54 (d, J=11.8 Hz, 1H), 3.51 (t, J=6.5 Hz, 1H), 1.94-1.90 (m, 1H), 1.84-1.76 (m, 2H), 1.59-1.56 (m, 1H), 1.28 (s, 3H).

A mixture of cis-2-(benzyloxy)-1-methylcyclopentan-4,4-d₂-1-ol (III-J, 70 mg, 0.30 mmol) and palladium (10% on carbon, 70 mg) in methanol (5 mL) was stirred at room temperature under one atmosphere of H₂. After completion, the reaction mixture was filtered, and the filtrate was concentrated to give cis-1-methylcyclopentane-4,4-d₂-1,2-diol (Intermediate III, 30 mg, 75%) as a yellow oil.

### Synthesis of cis-3-methyltetrahydrofuran-3,4-diol (Intermediate IV)

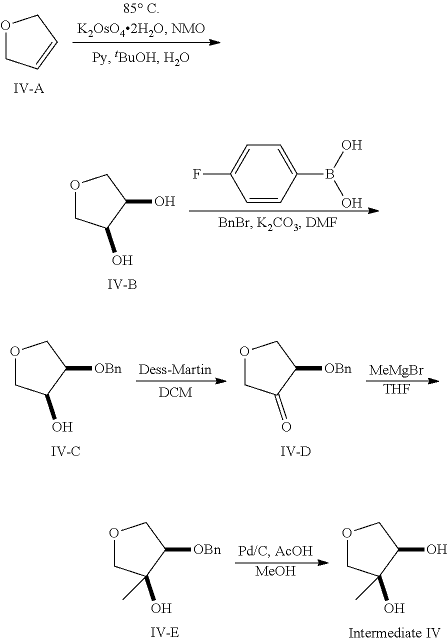

To a solution of 2,5-dihydrofuran (IV-A, 2.10 g, 30.0 mmol) in tert-butanol (27 mL) were added potassium dioxidodioxoosmium dihydrate (552 mg, 1.50 mmol), 4-methylmorpholine N-oxide (NMO) (4.80 g, 42.0 mmol), pyridine (2.40 mL, 30.0 mmol) and water (9 mL). The reaction mixture was stirred at 85° C. for 5 hours. After completion, the mixture was filtered through a Celite pad, and the filtrate was quenched with saturated NaHSO₃ aqueous solution (10 mL). The reaction mixture was concentrated under reduced pressure, and then separated using silica gel column chromatography eluted with methanol/dichloromethane (from 0 to 6%) to afford cis-tetrahydrofuran-3,4-diol (IV-B, 2.55 g, 82%) as a yellow oil.

To a mixture of cis-tetrahydrofuran-3,4-diol (IV-B, 1.65 g, 15.9 mmol) and benzyl bromide (BnBr) (2.85 mL, 23.8 mmol) in N,N-dimethylformamide (DMF) (18 mL) was added potassium carbonate (K₂CO₃) (3.29 g, 23.8 mmol), and the reaction mixture was stirred at room temperature overnight. After completion, the reaction was quenched with ice water (50 mL), and extracted with ethyl acetate (100 mL×3). The organic layers were collected, dried over Na₂SO₄, concentrated under reduced pressure, and then subjected to silica gel column chromatography eluted with ethyl acetate/petroleum ether (from 0%-30%) to afford cis-4-(benzyloxy)tetrahydrofuran-3-ol (IV-C, 2.26 g, 73%) as a colorless oil.

To a solution of cis-4-(benzyloxy)tetrahydrofuran-3-ol (IV-C, 2.26 g, 11.6 mmol) in dichloromethane (30 mL) was added Dess-Martin Periodinane (9.86 g, 23.3 mmol) carefully at 0° C. The reaction mixture was stirred at room temperature overnight. After completion, saturated sodium thiosulfate solution (20 mL) and saturated sodium carbonate solution (20 mL) were added at 0° C. to quench the reaction, and the mixture was extracted with dichloromethane (100 mL×2). The organic layers were collected, dried over Na₂SO₄, concentrated under reduced pressure to give a residue, which was separated using silica gel column chromatography eluted with ethyl acetate/petroleum ether (from 0 to 20%) to afford 4-(benzyloxy)dihydrofuran-3(2H)-one (IV-D, 1.35 g, 61%) as a colorless oil. ¹H NMR (500 MHz, DMSO-d₆) δ 7.41-7.29 (m, 5H), 4.76 (d, J=11.7 Hz, 1H), 4.62 (d, J=11.7 Hz, 1H), 4.35-4.28 (m, 1H), 4.20 (t, J=7.1 Hz, 1H), 4.07-4.00 (m, 1H), 3.96 (d, J=17.4 Hz, 1H), 3.82 (dd, J=9.6, 7.1 Hz, 1H).

To a solution of 4-(benzyloxy)dihydrofuran-3(2H)-one (IV-D, 1.12 g, 5.80 mmol) in dry THE (10 mL) was added methyl magnesium bromide (1 M in THF, 11.7 mL) at −20° C. under N₂ protection. The reaction mixture was stirred at 0° C. for 1 hour. After completion, saturated NH₄Cl solution (10 mL) was added at 0° C. to quench the reaction. The mixture was extracted with ethyl acetate (100 mL×2). The organic layers were collected, dried over Na₂SO₄, concentrated under reduced pressure, and then separated using silica gel column chromatography to afford cis-4-(benzyloxy)-3-methyltetrahydrofuran-3-ol (IV-E, 321 mg, 23%) as a colorless oil. ¹H NMR (500 MHz, DMSO-d₆) δ 7.40-7.32 (m, 4H), 7.32-7.26 (m, 1H), 4.69 (d, J=12.1 Hz, 1H), 4.61 (s, 1H), 4.55 (d, J=12.2 Hz, 1H), 3.96-3.87 (m, 1H), 3.67-3.58 (m, 2H), 3.54 (d, J=8.3 Hz, 1H), 3.45 (d, J=8.3 Hz, 1H), 1.22 (s, 3H).

To a solution of cis-4-(benzyloxy)-3-methyltetrahydrofuran-3-ol (IV-E, 321 mg, 1.54 mmol) in methanol (20 mL) were added palladium (10% on carbon, 100 mg) and acetic acid (1 drop). The reaction mixture was stirred under one atmosphere of H₂ overnight. The mixture was filtered, and the filtrate was concentrated under reduced pressure to afford cis-3-methyltetrahydrofuran-3,4-diol (Intermediate IV, 158 mg, 87%) as a colorless oil.

### Synthesis of cis-tetrahydro-2H-pyran-3,4-diol (Intermediate V)

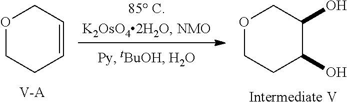

To a solution of (cyclopent-3-en-1-yloxy)tris(propan-2-yl)silane (V-A, 1.50 g, 6.24 mmol) in tert-butanol (5 mL) were added potassium osmate (22 mg, 0.06 mmol), 4-methylmorpholine N-oxide (NMO) (195 mg, 1.67 mmol), pyridine (96 uL, 1.20 mmol) and water (1.5 mL). The reaction mixture was stirred at 85° C. for 5 hours. The reaction mixture was concentrated to give a residue, which was separated using silica gel column chromatography eluted with methanol/dichloromethane (from 0 to 10%) to give cis-tetrahydro-2H-pyran-3,4-diol (Intermediate V, 1.30 g, 76%) as a yellow oil.

¹H NMR (500 MHz, CDCl₃) δ 3.89-3.82 (m, 3H), 3.78-3.76 (m, 1H), 3.56-3.52 (m, 1H), 3.48-3.43 (m, 1H), 3.03 (s, 2H), 1.90-1.83 (m, 1H), 1.80-1.75 (m, 1H).

### Synthesis of cis-5,5-difluoro-1-methylcyclohexane-1,2-diol (Intermediate VI)

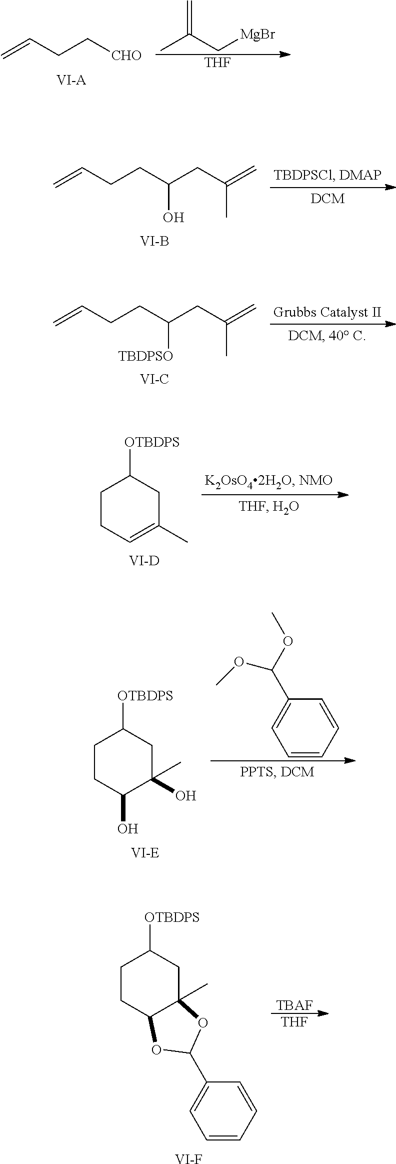

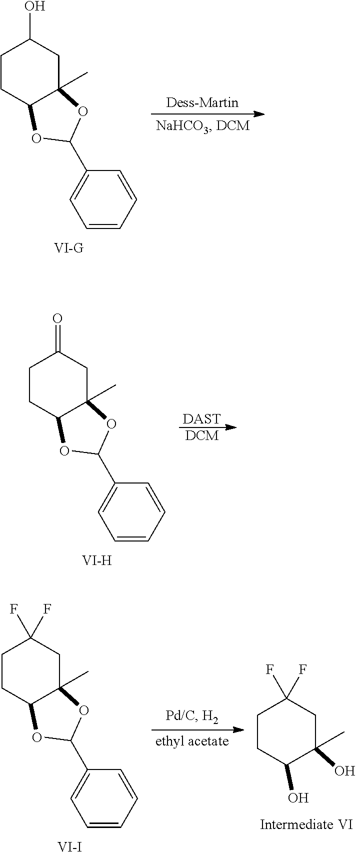

To a solution of pent-4-enal (VI-A, 10.0 g, 119 mmol) in THF (100 mL) was added (2-methylallyl)magnesium bromide (0.5 M in THF, 286 mL, 143 mmol) at 0° C. The reaction mixture was stirred at 25° C. under N₂ atmosphere for 1 hour. After completion, the reaction mixture was quenched with H₂O (200 mL) at 0° C. and extracted with EtOAc (100 mL×3). The combined organic layers were washed with brine (50 mL), dried over Na₂SO₄, and concentrated under reduced pressure to afford 2-methylocta-1,7-dien-4-ol (VI-B, 16.2 g, 97%) as a colorless oil.

To a solution of 2-methylocta-1,7-dien-4-ol (VI-B, 16.2 g, 116 mmol) in dichloromethane (500 mL) were added tert-butyldiphenylchlorosilane (TBDPSCl) (47.6 g, 173 mmol) and N,N-dimethylpyridin-4-amine (DMAP) (28.2 g, 231 mmol), and the reaction mixture was stirred at room temperature overnight. After completion, the reaction mixture was diluted with H₂O (300 mL) and extracted with dichloromethane (200 mL×3). The combined organic layers were washed with brine (200 mL), dried over Na₂SO₄, and concentrated under reduced pressure to yield a residue. The residue was separated using silica gel column chromatography to afford tert-butyl[(2-methylocta-1,7-dien-4-yl)oxy]diphenylsilane (VI-C, 39.1 g, 89%) as a colorless oil. ¹H NMR (400 MHz, CDCl₃) δ 7.61-7.0 (m, 4H), 7.30-7.26 (m, 6H), 5.60-5.53 (m, 1H), 4.82-4.75 (m, 2H), 4.56-4.51 (m, 2H), 3.79-3.16 (m, 1H), 2.08-2.04 (m, 2H), 2.04-1.98 (m, 2H), 1.40-1.35 (m, 2H), 1.32 (s, 3H), 0.97 (s, 9H).

To a solution of tert-butyl[(2-methylocta-1,7-dien-4-yl)oxy]diphenylsilane (VI-C, 39.1 g, 103 mmol) in dichloromethane (500 mL) was added Grubbs Catalyst 11 (4.38 g, 5.16 mmol). The reaction mixture was stirred at 40° C. under N₂ atmosphere overnight. After completion, the reaction mixture was diluted with H₂O (100 mL) and extracted with dichloromethane (100 mL×3). The combined organic layers were washed with brine (100 mL), dried over Na₂SO₄, and concentrated under reduced pressure. The resulting residue was separated using silica gel column chromatography to afford tert-butyl[(3-methylcyclohex-3-en-1-yl)oxy]diphenylsilane (VI-D, 32.1 g, 89%) as a colorless oil. ¹H NMR (400 MHz, CDCl₃) δ 7.70-7.67 (m, 4H), 7.42-7.35 (m, 6H), 5.29 (s, 1H), 3.96-3.94 (m, 1H), 2.12-2.03 (m, 3H), 1.86-1.85 (m, 1H), 1.66-1.61 (m, 1H), 1.58-1.52 (m, 4H), 1.07 (s, 9H).

To a solution of tert-butyl[(3-methylcyclohex-3-en-1-yl)oxy]diphenylsilane (VI-D, 32.1 g, 91.4 mmol) in THF (300 mL) and H₂O (30 mL) were added potassium dioxidodioxoosmium dihydrate (1.68 g, 4.56 mmol) and 4-methylmorpholine N-oxide (NMO) (12.9 g, 110 mmol) and the reaction mixture was stirred at 25° C. overnight. After completion, the reaction mixture was quenched with saturated NaHSO₃ solution (50 mL) and H₂O (150 mL), and extracted with EtOAc (100 mL×3). The combined organic layers were washed with brine (100 mL), dried over Na₂SO₄, and concentrated under reduced pressure to yield a residue, which was separated using silica gel column chromatography to give cis-5-((tert-butyldiphenylsilyl)oxy)-1-methylcyclohexane-1,2-diol (VI-E, 30.3 g, 86%) as a colorless oil.

To a mixture of cis-5-((tert-butyldiphenylsilyl)oxy)-1-methylcyclohexane-1,2-diol (VI-E, 30.3 g, 78.8 mmol) and (dimethoxymethyl)benzene (24.0 g, 157 mmol) in dichloromethane (300 mL) was added pyridinium p-toluenesulfonate (PPTS) (3.96 g, 15.8 mmol), and the reaction mixture was stirred at 25° C. overnight. After completion, the reaction mixture was diluted with H₂O (100 mL) and extracted with dichloromethane (100 mL×3). The combined organic layers were washed with brine (100 mL), dried over Na₂SO₄, and concentrated under reduced pressure to yield a residue. The residue was separated using silica gel column chromatography to afford cis-tert-butyl((3a-methyl-2-phenylhexahydrobenzo[d][1,3]dioxol-5-yl)oxy)diphenylsilane (VI-F, 25.2 g, 67%) as a colorless oil. ¹H NMR (400 MHz, CDCl₃) δ 7.66-7.65 (m, 4H), 7.43-7.26 (m, 11H), 5.93-5.89 (m, 1H), 4.21-4.14 (m, 1H), 3.95-3.90 (m, 1H), 2.16-2.10 (m, 1H), 1.95-1.92 (m, 1H), 1.85-1.76 (m, 2H), 1.62-1.55 (m, 4H), 1.38-1.36 (m, 1H), 1.08 (s, 9H).

To a solution of cis-tert-butyl((3a-methyl-2-phenylhexahydrobenzo[d][1,3]dioxol-5-yl)oxy)diphenylsilane (VI-F, 25.2 g, 53.3 mmol) in THE (300 mL) was added tetrabutylammonium fluoride (TBAF) (20.9 g, 80.0 mmol), and the reaction mixture was stirred at 70° C. for 2 hours. After completion, the reaction mixture was diluted with H₂O (100 mL) and extracted with EtOAc (120 mL×3). The combined organic layers were washed with brine (100 mL×5), dried over Na₂SO₄ and concentrated under reduced pressure to yield a residue, which was subjected to silica gel column chromatography to afford cis-3a-methyl-2-phenyl-hexahydro-2H-1,3-benzodioxol-5-ol (VI-G, 12.1 g, 97%) as a colorless oil.

To a solution of cis-3a-methyl-2-phenyl-hexahydro-2H-1,3-benzodioxol-5-ol (VI-G, 12.1 g, 51.6 mmol) in dichloromethane (200 mL) were added sodium hydrogen carbonate (8.68 g, 103 mmol) and Dess-Martin (32.9 g, 77.5 mmol) at 0° C. The reaction mixture was stirred at room temperature under N₂ for 2 hours. After completion, the reaction mixture was quenched with saturated solution of Na₂S203 (100 mL) and extracted with dichloromethane (100 mL×2). The combined organic layers were washed with brine (100 mL), dried over Na₂SO₄, and concentrated under reduced pressure. The resulting residue was separated using silica gel column chromatography to afford cis-3a-methyl-2-phenyltetrahydrobenzo[d][1,3]dioxol-5(4H)-one (VI-H, 10.4 g, 87%) as a colorless oil. ¹H NMR (400 MHz, CDCl₃) δ 7.42-7.37 (m, 5H), 5.80 (s, 1H), 4.26 (s, 1H), 2.79-2.75 (m, 1H), 2.60-2.42 (m, 2H), 2.28-2.24 (m, 2H), 2.03-1.95 (m, 1H), 1.48 (s, 3H).

To a solution of cis-3a-methyl-2-phenyltetrahydrobenzo[d][1,3]dioxol-5(4H)-one (VI-H, 5.00 g, 21.5 mmol) in dichloromethane (20 mL) was added diethylaminosulfur trifluoride (DAST) (20 mL) at 0° C. The reaction mixture was stirred at room temperature under N₂ overnight. After completion, the reaction was quenched with water (50 mL) and extracted with ethyl acetate (50 mL×2). The combined organic layers were washed with brine (20 mL), dried over Na₂SO₄, and concentrated under reduced pressure to yield a residue, which was separated using silica gel column chromatography to afford cis-5,5-difluoro-3a-methyl-2-phenylhexahydrobenzo[d][1,3]dioxole (VI-I, 3.50 g, 64%) as a colorless oil.

To a solution of cis-5,5-difluoro-3a-methyl-2-phenylhexahydrobenzo[d][1,3]dioxole (VI-I, 3.50 g, 13.8 mmol) in ethyl acetate (100 mL) was added palladium (10% on carbon, 500 mg) and the reaction mixture was stirred at room temperature under H₂ (1 atm) overnight. After completion, the mixture was filtered through a short pad of Celite© and the filtrate was concentrated under reduced pressure to yield a residue. The residue was separated using silica gel column chromatography to afford cis-5,5-difluoro-1-methylcyclohexane-1,2-diol (Intermediate VI, 1.80 g, 79%) as a white solid. ¹H NMR (400 MHz, DMSO-d₆) δ 4.62-4.61 (m, 1H), 4.23 (s, 1H), 3.37-3.36 (m, 1H), 2.02-1.98 (m, 1H), 1.85-1.73 (m, 4H), 1.59-1.57 (m, 1H), 1.11 (s, 3H). ¹⁹F NMR (400 MHz, DMSO-d₆) δ −86.9 & −87.6 (d), −89.3 & −89.9 (d).

### Synthesis of oxepane-4,5-diol (Intermediate VII)

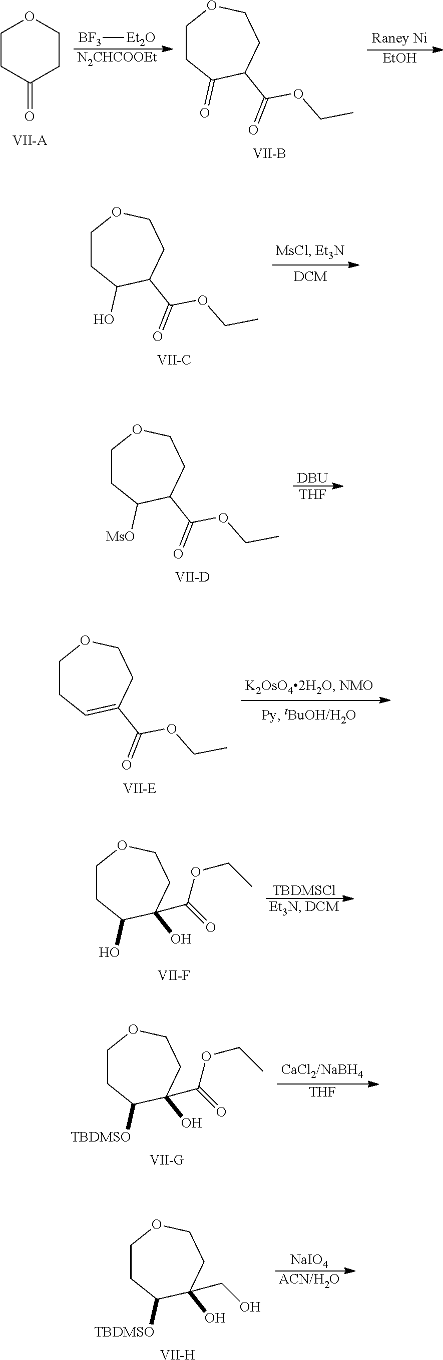

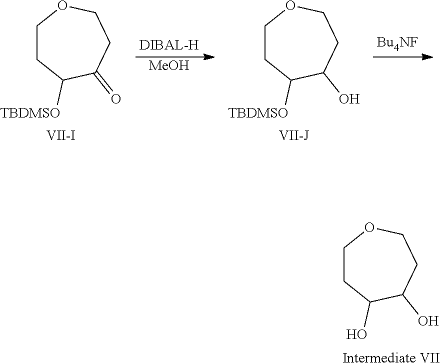

To a solution of oxan-4-one (VII-A, 1.53 g, 15.3 mmol) in THE (10 mL) were added ethyl diazoacetate (1.62 mL, 15.4 mmol) and boron trifluoride ethyl ether (1.80 mL, 15.3 mmol) at −30° C. over 15 mins. The reaction was stirred for 1 hour at that temperature. Then it was quenched with 30% Na₂CO₃ aqueous solution slowly. The organic phase was separated, and the aqueous phase was extracted with ethyl acetate (100 mL×3). The combined organic extracts were dried over Na₂SO₄, and concentrated under reduced pressure. The residue was separated using silica gel column chromatography eluted with petroleum ether/ethyl acetate (from 1:0 to 5:1) to afford ethyl 5-oxooxepane-4-carboxylate (VII-B, 1.36 g, 48%) as a colorless oil. LC-MS (ESI): m/z 187.1 [M+H]⁺.

To a solution of ethyl 5-oxooxepane-4-carboxylate (VII-B, 3.70 g, 19.9 mmol) in EtOH (25 mL) at 0° C. was added Raney Nickel (430 mg, 1.99 mmol). The resulting mixture was stirred at 50° C. for 12 hours. It was then filtered and the filtrate was concentrated under reduced pressure to yield a residue. The residue was subjected to silica gel column chromatography to give ethyl 5-hydroxyoxepane-4-carboxylate (VII-C, 3.40 g, 91%) as a colorless oil. LC-MS (ESI): m/z 189.1 [M+H]. ¹H NMR (500 MHz, CDCl₃) δ 4.31-4.29 (m, 1H), 4.20 (q, J=7.0 Hz, 2H), 3.89-3.84 (m, 1H), 3.80-3.71 (m, 2H), 3.68-3.64 (m, 1H), 3.13 (br s, 1H), 2.81-2.78 (m, 1H), 2.47-2.40 (m, 1H), 2.00-1.95 (m, 1H), 1.91-1.80 (m, 2H), 1.30 (t, J=7.0 Hz, 3H).

To a solution of ethyl 5-hydroxyoxepane-4-carboxylate (VII-C, 2.82 g, 15.0 mmol) in dry dichloromethane (30 mL) at 0° C. were added triethyl amine (3.0 mL) and methanesulfonyl chloride (2.0 mL, 22.5 mmol). The mixture was stirred at room temperature for 5 hours. The reaction was quenched with saturated NaHCO₃ solution (50 mL) and extracted with dichloromethane (100 mL×3). The combined organic extracts were dried over Na₂SO₄ and concentrated under reduced pressure. The residue was separated using silica gel column chromatography to give ethyl 5-((methylsulfonyl)oxy)oxepane-4-carboxylate (VII-D, 3.67 g, 92%) as a colorless oil. LC-MS (ESI): m/z 267.1 [M+H]⁺.

To a solution of ethyl 5-((methylsulfonyl)oxy)oxepane-4-carboxylate (VII-D, 3.87 g, 14.5 mmol) in THE (30 mL) was added 1,8-diazabicyclo[5.4.0]undec-7-ene (DBU) (3.0 mL) at room temperature. The reaction mixture was stirred for 4 hours before it was diluted with EtOAc (50 mL) and washed with brine (100 mL). The organic layer was dried over anhydrous Na₂SO₄, and concentrated under reduced pressure. The residue was separated using silica gel column chromatography to afford ethyl 2,3,6,7-tetrahydrooxepine-4-carboxylate (VII-E, 1.27 g, 51%) as a colorless oil. ¹H NMR (500 MHz, CDCl₃) δ 7.22 (t, J=6.0 Hz, 1H), 4.21 (q, J=7.0 Hz, 2H), 3.72-3.70 (m, 4H), 2.78-2.75 (m, 2H), 2.52-2.49 (m, 2H), 1.31 (t, J=6.0 Hz, 3H).

A mixture of ethyl 2,3,6,7-tetrahydrooxepine-4-carboxylate (VII-E, 1.07 g, 6.29 mmol), potassium osmate dihydrate (120 mg, 0.37 mmol), 4-methylmorpholine N-oxide (NMO) (1.20 g, 10.3 mmol), pyridine (0.8 mL), H₂O (7 mL) and t-BuOH (20 mL) was stirred under N₂ atmosphere at 80° C. overnight. After completion, the mixture was cooled to room temperature, filtered through a Celite pad, and the pad was washed with methanol (HPLC grade, 30 mL). The filtrate was concentrated under reduced pressure to give a residue, which was separated using silica gel column chromatography to give cis-ethyl 4,5-dihydroxyoxepane-4-carboxylate (VII-F, 1.01 g, 78%). ¹H NMR (500 MHz, CDCl₃) δ 4.32 (q, J=7.0 Hz, 2H), 4.20 (d, J=10 Hz, 1H), 3.84-3.80 (m, 2H), 3.78-3.71 (m, 2H), 3.56 (s, 1H), 2.45-2.39 (m, 1H), 2.20-2.5 (m, 2H), 1.85-1.81 (m, 1H), 1.75-1.71 (m, 1H), 1.34 (t, J=7.0 Hz, 3H).

To a mixture of cis-ethyl 4,5-dihydroxyoxepane-4-carboxylate (VII-F, 710 mg, 3.50 mmol), imidazole (790 mg, 11.6 mmol) and triethylamine (1.2 mL) in dichloromethane (30 mL) was added tert-butyl dimethyl chlorosilane (TBDMSCl) (1.26 g, 8.38 mmol) at 0° C. The reaction mixture was warmed to and stirred at 80° C. for 12 hours before it was quenched with saturated NaHCO₃ solution (50 mL) and extracted with dichloromethane (60 mL×3). The combined organic extracts were dried over Na₂SO₄ and concentrated under reduced pressure. The residue was separated using silica gel column chromatography to give ethyl 5-[(tert-butyldimethylsilyl)oxy]-4-hydroxyoxepane-4-carboxylate (VII-G, 0.74 g, 68%). ¹H NMR (500 MHz, CDCl₃) δ 4.27 (q, J=7.0 Hz, 2H), 4.16-4.09 (m, 1H), 3.84-3.78 (m, 2H), 3.74-3.68 (m, 2H), 3.33 (br s, 1H), 2.52-2.44 (m, 1H), 2.20-2.15 (m, 1H), 1.79-1.75 (m, 1H), 1.65-1.61 (m, 1H), 1.32 (t, J=7.0 Hz, 3H) 0.86 (s, 9H), 0.08 (s, 3H), 0.01 (s, 3H).

To a mixture of cis-ethyl 5-[(tert-butyldimethylsilyl)oxy]-4-hydroxyoxepane-4-carboxylate (VII-G, 1.33 g, 4.20 mmol) and CaCl₂) (930 mg, 8.40 mmol) in THE (12 mL) at 0° C. was added NaBH₄ (560 mg, 16.7 mmol). The mixture was stirred for 15 mins and then allowed to warm gradually to room temperature and stirred for 12 hours. The reaction was quenched with saturated NaHCO₃ solution (10 mL) and extracted with dichloromethane (60 mL×3). The combined organic extracts were dried over Na₂SO₄ and concentrated under reduced pressure to yield a residue, which was separated using silica gel column chromatography to give 5-[(tert-butyldimethylsilyl)oxy]-4-(hydroxymethyl)oxepan-4-ol (VII-I, 1.12 g, 97%). LC-MS (ESI): m/z 277.2 [M+H]⁺.

To a solution of 5-[(tert-butyldimethylsilyl)oxy]-4-(hydroxymethyl)oxepan-4-ol (VII-I, 64 mg, 0.23 mmol) in acetonitrile (1 mL) and H₂O (0.1 mL) was added NaIO₄ (50 mg, 0.23 mmol), and the mixture was stirred at room temperature for 3 hours. Then ethyl acetate (15 mL) and a saturated aqueous solution of Na₂SO₃ (8 mL) were added. The mixture was vigorously stirred for 15 mins, and then the two phases were separated using a separatory funnel. The aqueous solution was extracted with ethyl acetate (50 mL×2). The organic layers were combined, washed with brine (20 mL), dried over anhydrous MgSO₄, and concentrated under reduced pressure. The residue was separated using silica gel column chromatography to give 5-[(tert-butyldimethylsilyl)oxy]oxepan-4-one (VII-I, 46 mg, 81%). ¹H NMR (500 MHz, CDCl₃) δ 4.39 (dd, J=7.0, 2.0 Hz, 1H), 4.10-4.05 (m, 1H), 3.99-3.95 (m, 1H), 3.92-3.89 (m, 2H), 2.85-2.80 (m, 1H), 2.72-2.66 (m, 1H), 1.91-1.84 (m, 1H), 1.80-1.74 (m, 1H), 0.95 (s, 9H), 0.11 (s, 6H).

To a solution of 5-[(tert-butyldimethylsilyl)oxy]oxepan-4-one (VII-I, 85 mg, 0.35 mmol) in THE (1 mL) was added DIBAL-H (1 M in hexane, 1.04 mL, 1.04 mmol) at 0° C. The mixture was warmed to room temperature and stirred for 2 hours. The resulting solution was filtered through Celite, washed with dichloromethane (20 mL), and concentrated under reduced pressure. The residue was separated using silica gel column chromatography to give 5-((tert-butyldimethylsilyl)oxy)oxepan-4-ol (VII-J, 75 mg, 87%). ¹H NMR (500 MHz, CDCl₃) δ 3.85-3.80 (m, 1H), 3.78-3.72 (m, 1H), 3.71-3.62 (m, 4H), 2.10-2.01 (m, 1H), 1.97-1.91 (m, 1H), 1.83-1.74 (m, 2H), 0.92 (s, 9H), 0.12 (s, 3H), 0.11 (s, 3H).

To a solution of 5-[(tert-butyldimethylsilyl)oxy]oxepan-4-ol (VII-J, 23 mg, 0.09 mmol) in dry THE (1 mL) was added TBAF (1 M in THF, 90 uL, 0.09 mmol) at 0° C., and the resulting solution stirred for 45 mins, allowing the mixture to warm to room temperature. The resulting solution was diluted with dichloromethane (20 mL) and quenched with water (5 mL). The organic layer was washed with brine (5 mL), dried over NaSO₄, and concentrated under reduced pressure. The crude product was separated using silica gel column chromatography to give oxepane-4,5-diol (Intermediate VII, 12 mg, 90%) as a mixture of isomers. LC-MS (ESI): m/z 133.1 [M+H]⁺.

### Synthesis of cis-oxepane-3,4-diol (Intermediate VIII)

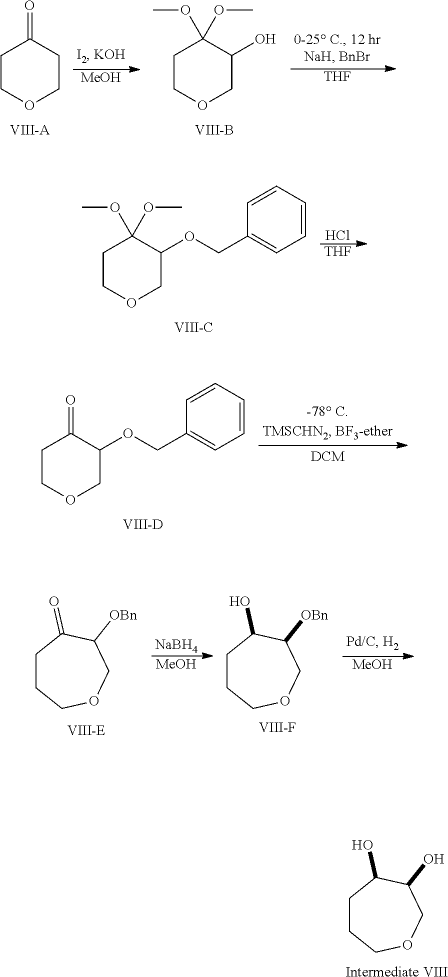

Oxan-4-one (VIII-A, 20.0 g, 200 mmol) and potassium hydroxide (22.4 g, 400 mmol) were dissolved in methanol (320 mL) under nitrogen atmosphere. The resulting solution was cooled to 0° C. and a solution of iodine (45.6 g, 180 mmol) in methanol (320 mL) was added dropwise over a period of 2 hours. Afterwards the reaction mixture was allowed to warm to room temperature and stirred for 1 hour. Then the solvent was removed under reduced pressure and the residue was suspended in ethyl acetate (500 mL). After filtration, the filtrate was concentrated to give a crude product which was separated using silica gel column chromatography eluted with petroleum ether/ethyl acetate (from 10:1 to 0:1) to give 4,4-dimethoxytetrahydro-2H-pyran-3-ol (VIII-B, 13.4 g, 41%) as a yellow oil. ¹H NMR (500 MHz, CDCl₃) δ 3.85-3.78 (m, 2H), 3.73-3.66 (m, 2H), 3.52-3.47 (m, 1H), 3.28 (s, 3H), 3.26 (s, 3H), 1.98-1.92 (m, 1H), 1.79-1.75 (m, 1H).

To a solution of 4,4-dimethoxytetrahydro-2H-pyran-3-ol (VIII-B, 2.70 g, 16.7 mmol) in tetrahydrofuran (50 mL) was added sodium hydride (0.87 g, 21.6 mmol) at 0° C. To the mixture was added (bromomethyl)benzene (2.38 mL, 20.0 mmol) dropwise and the reaction mixture was stirred at room temperature for 12 hours. Then it was quenched with water (20 mL) and extracted with ethyl acetate (20 mL×3). The combined organic layers were concentrated to give a residue which was separated using silica gel column chromatography eluted with petroleum ether/ethyl acetate (from 20:1 to 1:1) to give 3-(benzyloxy)-4,4-dimethoxytetrahydro-2H-pyran (VIII-C, 4.20 g, 100%) as a yellow oil. 1H NMR (500 MHz, CDCl₃) δ 7.42-7.28 (m, 5H), 4.77 (d, J=12.1 Hz, 1H), 4.64 (d, J=12.1 Hz, 1H), 3.99 (dd, J=12.3, 3.0 Hz, 1H), 3.85-3.81 (m, 1H), 3.63-3.60 (m, 1H), 3.56-3.51 (m, 1H), 3.44-3.42 (m, 1H), 3.24 (s, 3H), 3.22 (s, 3H), 2.12-2.07 (m, 1H), 1.79-1.75 (m, 1H).

To a solution of 3-(benzyloxy)-4,4-dimethoxytetrahydro-2H-pyran (VIII-C, 4.20 g, 16.7 mmol) in tetrahydrofuran (40 mL) was added hydrochloric acid (2 M solution, 41.6 mL), and the mixture was stirred at room temperature for 12 hours. The resulting mixture was adjusted to pH 7 with saturated sodium carbonate and extracted with ethyl acetate (50 mL×3). The combined organic layers were concentrated to give a residue which was separated using silica gel column chromatography eluted with petroleum ether/ethyl acetate (from 50:1 to 1:1) to give 3-(benzyloxy)tetrahydro-4H-pyran-4-one (VIII-D, 2.90 g, 84%) as a yellow oil. 1H NMR (500 MHz, CDCl₃) δ 7.40-7.30 (m, 5H), 4.87 (d, J=11.9 Hz, 1H), 4.56 (d, J=11.9 Hz, 1H), 4.21-4.17 (m, 1H), 4.17-4.07 (m, 1H), 4.02-3.99 (m, 1H), 3.76-3.71 (m, 1H), 3.62-3.58 (m, 1H), 2.62-2.60 (m, 2H).

To a solution of 3-(benzyloxy)tetrahydro-4H-pyran-4-one (VIII-D, 2.30 g, 11.2 mmol) in dichloromethane (40 mL) were added BF₃-ether complex (5.6 mL, 44.6 mmol) and (trimethylsilyl)diazomethane solution (2 M in hexane, 16.7 ml, 33.5 mmol) at −78° C. The reaction mixture was stirred at −78° C. for 1 hour, quenched with saturated sodium bicarbonate solution (2.6 mL) and water (20 mL), and extracted with dichloromethane (20 mL×3). The combined organic extracts were washed with brine, dried over sodium sulphate, and concentrated under reduced pressure to yield a residue. The residue was redissolved in methanol (4 mL) and to the resulting solution was added pyridin-1-ium 4-methylbenzenesulfonate (4.20 g, 16.7 mmol). After stirred at 25° C. for 1 hour, the reaction mixture was concentrated under reduced pressure followed by addition of water (50 mL) and then extracted with ethyl acetate (20 mL×3). The combined organic layers were washed with brine, dried over sodium sulphate, and concentrated under reduced pressure to yield a residue. The residue was separated using silica gel column chromatography eluted with petroleum ether/ethyl acetate (from 20:1 to 0:1) to give 3-(benzyloxy)oxepan-4-one (VIII-E, 390 mg, 16%) as a yellow oil. ¹H NMR (500 MHz, CDCl₃) δ 7.42-7.32 (m, 5H), 4.74 (d, J=11.8 Hz, 1H), 4.52 (d, J=11.8 Hz, 1H), 4.12 (t, J=5.1 Hz, 1H), 3.96-3.93 (m, 1H), 3.85-3.78 (m, 3H), 2.79-2.73 (m, 1H), 2.57-2.48 (m, 1H), 2.03-1.92 (m, 1H), 1.91-1.81 (m, 1H).

To a solution of 3-(benzyloxy)oxepan-4-one (VIII-E, 1.40 g, 6.36 mmol) in methanol (20 mL) was added sodium borohydride (430 mg, 12.7 mmol) at 0° C., and the mixture was stirred at room temperature for 1 hour. The reaction mixture was concentrated to give a residue, which was separated using silica gel column chromatography eluted with petroleum ether/ethyl acetate (from 10:1 to 1:1) to give cis-3-(benzyloxy)oxepan-4-ol (VIII-F, 660 mg, 47%) as a yellow oil. ¹H NMR (500 MHz, CDCl₃) δ 7.41-7.30 (m, 5H), 4.69-4.58 (m, 2H), 4.13-4.08 (m, 1H), 3.79-3.66 (m, 4H), 3.59-3.61 (m, 1H), 2.52 (d, J=4.3 Hz, 1H), 2.11-2.01 (m, 2H), 1.74-1.70 (m, 1H), 1.62-1.56 (m, 1H).

To a solution of cis-3-(benzyloxy)oxepan-4-ol (VIII-F, 700 mg, 3.15 mmol) in methanol (2 mL) was added palladium (10% on carbon, 335 mg), and the mixture was stirred at room temperature for 1 hour. The resulting mixture was filtered, and the filtrate was concentrated under reduced pressure to give a crude product, which was separated using silica gel column chromatography eluted with petroleum ether/ethyl acetate (from 10:1 to 0:1) to give cis-oxepane-3,4-diol (Intermediate VIII, 320 mg, 77%) as a yellow oil. ¹H NMR (500 MHz, CDCl₃) δ 3.85 (s, 1H), 3.79-3.73 (m, 4H), 3.63-3.60 (m, 1H), 2.71-2.70 (m, 1H), 2.70-2.56 (m, 1H), 1.91-1.84 (m, 2H), 1.80-1.74 (m, 1H), 1.70-1.63 (m, 1H).

### Synthesis of cis-3-fluoro-1-(methylsulfonyl)piperidin-4-amine (Intermediate IX)

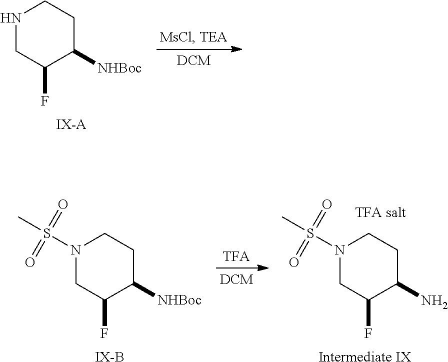

To a mixture of cis-tert-butyl (3-fluoropiperidin-4-yl)carbamate (IX-A, 480 mg, 2.20 mmol) and triethylamine (667 mg, 6.60 mmol) in dichloromethane (10 mL) was added methanesulfonyl chloride (298 mg, 2.60 mmol) at 0° C. The reaction mixture was stirred at room temperature under N₂ for 1 hour. The reaction mixture was diluted with H₂O (50 mL) and extracted with dichloromethane (50 mL×2). The combined organic layers were washed with brine (30 mL), dried over Na₂SO₄, and concentrated under reduced pressure. The resulting residue was separated using silica gel column chromatography to afford cis-tert-butyl (3-fluoro-1-(methylsulfonyl)piperidin-4-yl)carbamate (IX-B, 603 mg, 93%) as a white solid. LC-MS (ESI): m/z 297.1 [M+H]⁺.

To a solution of cis-tert-butyl (3-fluoro-1-(methylsulfonyl)piperidin-4-yl)carbamate (IX-B, 603 mg, 2.00 mmol) in dichloromethane (6 mL) was added trifluoroacetic acid (0.6 mL) at 0° C. The reaction mixture was stirred at room temperature under N₂ overnight. After completion, the reaction mixture was concentrated under reduced pressure to afford cis-3-fluoro-1-(methylsulfonyl)piperidin-4-amine (Intermediate IX, 380 mg, 61%) as TFA salt. LC-MS (ESI): m/z 197.1 [M+H]⁺.

### Synthesis of 1-((1-methyl-1H-pyrazol-4-yl)sulfonyl)piperidin-4-amine (Intermediate X)

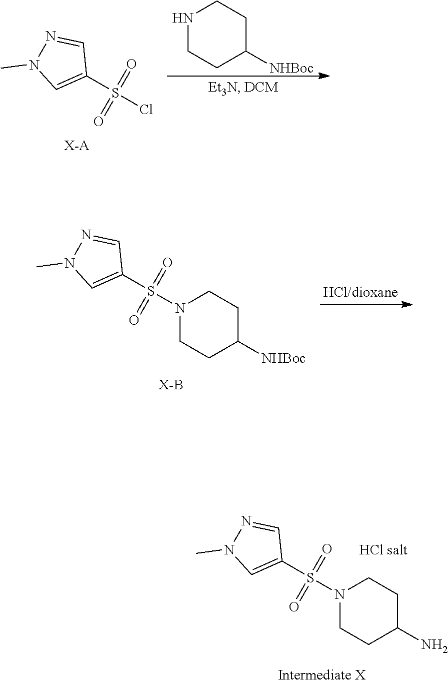

To a mixture of tert-butyl N-(piperidin-4-yl)carbamate (1.11 g, 5.50 mmol) and triethylamine (2.3 mL, 16.5 mmol) in dichloromethane (20 mL) was added 1-methyl-1H-pyrazole-4-sulfonyl chloride (X-A, 1.00 g, 5.50 mmol) at 0° C. The reaction mixture was stirred at room temperature under N₂ atmosphere for 1 hour. After completion, the mixture was diluted with H₂O (40 mL) and extracted with dichloromethane (40 mL×2). The combined organic layers were washed with brine (30 mL), dried over Na₂SO₄, and concentrated under reduced pressure. The residue was separated using silica gel column chromatography to afford tert-butyl (1-((1-methyl-1H-pyrazol-4-yl)sulfonyl)piperidin-4-yl)carbamate (X-B, 1.70 g, 89%) as a white solid. LC-MS (ESI): m/z 345.1 [M+H]⁺.

To a solution of tert-butyl (1-((1-methyl-1H-pyrazol-4-yl)sulfonyl)piperidin-4-yl)carbamate (X-B, 1.70 g, 4.94 mmol) in dioxane (20 mL) was added HCl (4 M in dioxane, 20 mL) at 0° C., and the reaction mixture was stirred at room temperature overnight. The reaction mixture was concentrated under reduced pressure to afford 1-((1-methyl-1H-pyrazol-4-yl)sulfonyl)piperidin-4-amine (Intermediate X, 1.10 g, 80%) as HCl salt. LC-MS (ESI): m/z 245.1 [M+H]⁺.

### Synthesis of 4-amino-N-(oxetan-3-yl)benzenesulfonamide (Intermediate XI)

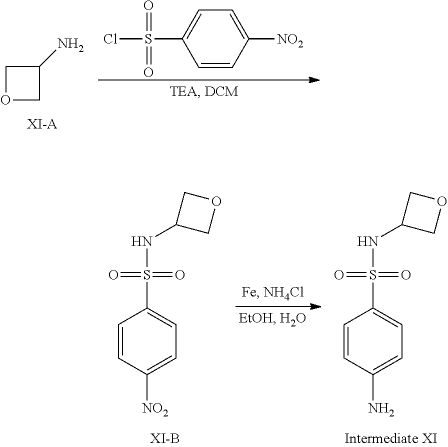

To a solution of 4-nitrobenzenesulfonyl chloride (300 mg, 1.35 mmol) in dichloromethane (5 mL) were added oxetan-3-amine (XI-A, 0.10 mL, 1.35 mmol) and triethylamine (0.60 mL, 4.06 mmol). The reaction mixture was stirred at room temperature for 2 hours. Then it was poured into ice water (10 mL) and extracted with dichloromethane (10 mL×3). The combined organic layers were washed with brine (10 mL), dried over Na₂SO₄ and concentrated under reduced pressure. The residue was separated using silica gel column chromatography to give 4-nitro-N-(oxetan-3-yl)benzenesulfonamide (XI-B, 250 mg, 72%) as a white solid. LC-MS (ESI): m/z 259.0 [M+H]⁺.

To a mixture of 4-nitro-N-(oxetan-3-yl)benzenesulfonamide (XI-B, 250 mg, 0.97 mmol) and iron powder (65 mg, 9.68 mmol) in EtOH (7 mL) and H₂O (3 mL) was added NH₄Cl (1026 mg, 19.4 mmol), and the reaction mixture was stirred at 60° C. overnight. The reaction mixture was filtered through a short pad of Celite*, and the filter cake was washed with EtOAc (30 mL). The filtrate was concentrated under reduced pressure, and the residue was separated using silica gel column chromatography to give 4-amino-N-(oxetan-3-yl)benzenesulfonamide (Intermediate XI, 200 mg, 91%) as a brown solid. LC-MS (ESI): m/z 229.1 [M+H]⁺.

### Synthesis of 4-amino-3-fluoro-N-(methyl-d₃)benzenesulfonamide (Intermediate XII)

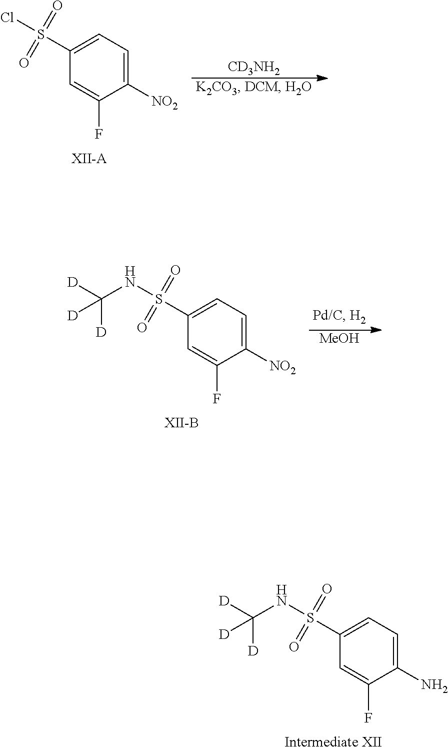

To a mixture of methyl-d₃-amine hydrochloride (397 mg, 5.63 mmol) and potassium carbonate (1.56 g, 11.3 mmol) in dichloromethane/water (15 mL/5 mL) was added 3-fluoro-4-nitrobenzene-1-sulfonyl chloride (XII-A, 900 mg, 3.76 mmol). The mixture was stirred at room temperature for 2 hours and then diluted with water (20 mL), followed by extraction with ethyl acetate (10 mL×3). The combined organic layers were washed with brine and concentrated under reduced pressure to give 3-fluoro-N-(methyl-d₃)-4-nitrobenzenesulfonamide (XII-B, 1.00 g, crude) as a yellow oil. ¹H NMR (500 MHz, CDCl₃) δ 8.22 (dd, J=8.8, 6.9 Hz, 1H), 7.95-7.61 (m, 2H), 4.70 (s, 1H).

A mixture of 3-fluoro-N-(methyl-d₃)-4-nitrobenzenesulfonamide (XII-B, 1.00 g, crude from last step) and palladium (10% on carbon, 0.45 g) in methanol (15 mL) was stirred at room temperature for 12 hours under one atmosphere of H₂. The reaction mixture was then filtered, and the filtrate was concentrated to give a residue, which was separated using silica gel column chromatography eluted with petroleum ether/ethyl acetate (from 10:1 to 1:1) to give 4-amino-3-fluoro-N-(methyl-d₃)benzenesulfonamide (Intermediate XII, 480 mg) as a yellow solid. ¹H NMR (500 MHz, CDCl₃) δ 7.54-7.43 (m, 2H), 6.84 (t, J=8.3 Hz, 1H), 4.22 (s, 3H).

### Synthesis of 4-amino-N-(tetrahydro-2H-pyran-4-yl)benzenesulfonamide (Intermediate XIII)

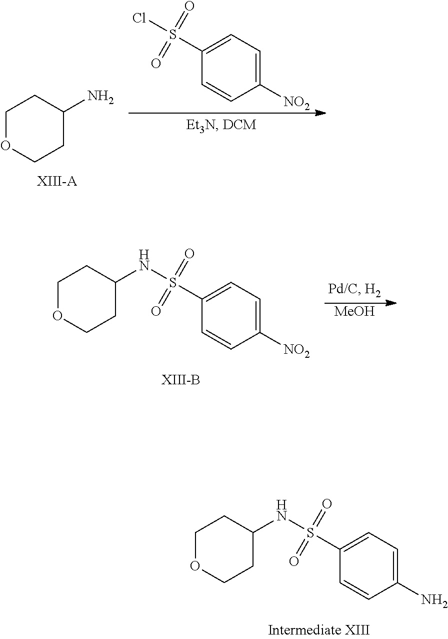

To a mixture of tetrahydro-2H-pyran-4-amine (XIII-A, 505 mg, 5.00 mmol) and 4-nitrobenzenesulfonyl chloride (1.10 g, 5.00 mmol) in dichloromethane (20 mL) was added triethylamine (1.38 mL, 10.0 mmol). The mixture was stirred at room temperature for 2 hours. It was concentrated under reduced pressure to give 4-nitro-N-(tetrahydro-2H-pyran-4-yl)benzenesulfonamide (XIII-B, crude), which was used in next step without further purification.

To the solution of the 4-nitro-N-(tetrahydro-2H-pyran-4-yl)benzenesulfonamide (XIII-B, crude from previous step) in methanol (20 mL) was added palladium (10% on carbon, 200 mg). The mixture was stirred at room temperature overnight under one atmosphere of H₂. Then it was filtered and the filtrate was concentrated under reduced pressure to give 4-amino-N-(tetrahydro-2H-pyran-4-yl)benzenesulfonamide (Intermediate XIII, 1.04 g, 82% from XIII-A). LC-MS (ESI): m/z 257.1 [M+H]⁺.

### Synthesis of 4-amino-N-(3-methyloxetan-3-yl)benzenesulfonamide (Intermediate XIV)

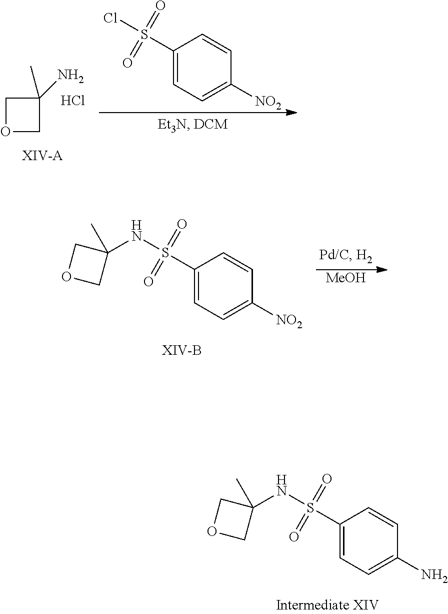

To a mixture of 3-methyloxetan-3-amine HCl salt (XIV-A, 0.41 g, 3.28 mmol) and triethylamine (1.4 mL, 10.1 mmol) in dichloromethane (30 mL) was added 4-nitrobenzene-1-sulfonyl chloride (0.75 g, 3.37 mmol). The reaction mixture was stirred at room temperature for 3 hours and then poured into water (200 ml) and filtered. The filter cake was washed with dichloromethane (30 mL). Then the filtrate was concentrated under reduced pressure. The residue was separated using silica gel column chromatography to give N-(3-methyloxetan-3-yl)-4-nitrobenzenesulfonamide (XIV-B, 0.89 g, 99%). LC-MS (ESI): m/z 271.0 [M−H]⁻.

To a solution of N-(3-methyloxetan-3-yl)-4-nitrobenzene-1-sulfonamide (XIV-B, 0.89 g, 3.27 mmol) in methanol (10 mL) was added palladium (10% on carbon, 0.32 g). The reaction mixture was stirred at room temperature for 2 hours. Then it was filtered through a short pad of Celite©. The filter cake was washed with EtOAc (30 mL), and the filtrate was concentrated under reduced pressure. The resulting residue was separated using silica gel column chromatography to give 4-amino-N-(3-methyloxetan-3-yl)benzene-1-sulfonamide (Intermediate XIV, 0.45 g, 57%).

¹H NMR (500 MHz, DMSO-d₆) δ 7.79 (s, 1H), 7.89 (d, J=8.5 Hz, 2H), 6.62 (d, J=8.5 Hz, 2H), 5.97 (s, 2H), 4.92 (d, J=6.0 Hz, 2H), 4.04 (d, J=6.0 Hz, 2H), 1.43 (s, 3H).

### Illustration 1. Synthesis of 4-((5-cyano-4-(cyclopentylmethoxy)pyrimidin-2-yl)amino)benzenesulfonamide (1)

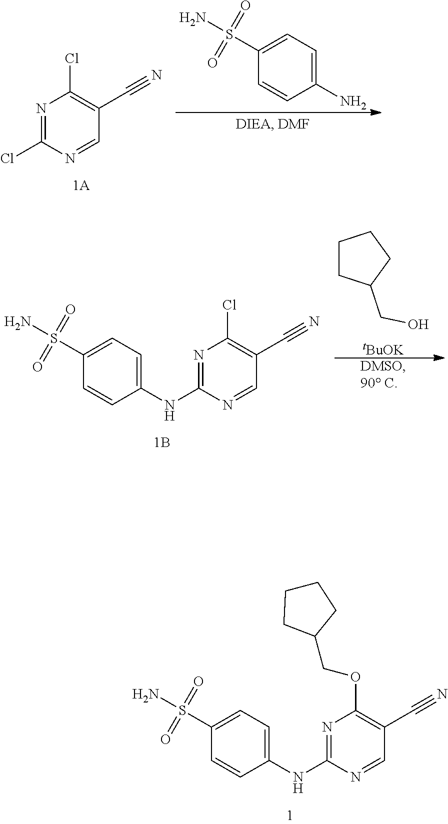

To a mixture of 2,4-dichloropyrimidine-5-carbonitrile (1A, 2.00 g, 11.5 mmol) and 4-aminobenzene-1-sulfonamide (2.18 g, 12.7 mmol) in anhydrous N,N-dimethylformamide (DMF) (20 mL) was added N,N-diisopropylethyl amine (DIEA) (4.46 g, 34.5 mmol) at 0° C. The reaction mixture was stirred at room temperature for 10 minutes before it was poured into water (200 mL). The precipitate formed was filtered, washed with water (30 mL×2), and dried to give 4-((4-chloro-5-cyanopyrimidin-2-yl)amino)benzenesulfonamide (1B, 1.60 g, 45%) as a yellow solid. LC-MS (ESI): m/z 310.0 [M+H]⁺. ¹H NMR (400 MHz, DMSO-d₆) δ 11.15 (s, 1H), 8.98 (s, 1H), 7.90-7.76 (m, 4H), 7.29 (s, 2H).

To a solution of 4-((4-chloro-5-cyanopyrimidin-2-yl)amino)benzenesulfonamide (1B, 60 mg, 0.19 mmol) and cyclopentylmethanol (58 mg, 0.58 mmol) in dimethyl sulfoxide (DMSO) (3 mL) was added potassium tert-butoxide (t-BuOK) (65 mg, 0.58 mmol), and the reaction mixture was stirred at 90° C. for 2 hours. Then it was poured into a cold saturated solution of NH₄Cl (15 mL) and extracted with ethyl acetate (20 mL×3). The combined organic layers were washed with brine (10 mL), dried over Na₂SO₄, and concentrated under reduced pressure to yield a residue. The residue was separated using prep-HPLC to give 4-((5-cyano-4-(cyclopentylmethoxy)pyrimidin-2-yl)amino)benzenesulfonamide (1, 16 mg, 23%). LC-MS (ESI): m/z 374.1 [M+H]⁺.

¹H NMR (400 MHz, DMSO-d₆) δ 10.65 (br s, 1H), 8.75 (s, 1H), 7.90 (d, J=8.8 Hz, 2H), 7.79 (d, J=8.8 Hz, 2H), 7.26 (s, 2H), 4.39 (d, J=7.2 Hz, 2H), 2.45-2.39 (m, 1H), 1.79-1.77 (m, 2H), 1.65-1.55 (m, 4H), 1.36-1.26 (m, 2H).

### Illustration 2. Synthesis of cis-4-((5-cyano-4-((2-hydroxycyclohexyl)oxy)pyrimidin-2-yl)amino)benzenesulfonamide (2)

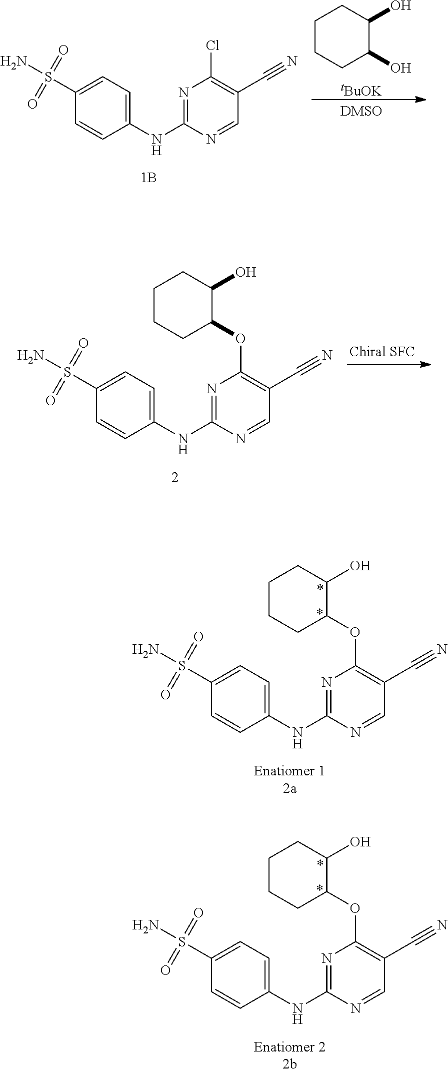

To a solution of 4-((4-chloro-5-(trifluoromethyl)pyrimidin-2-yl)amino)benzenesulfonamide (1B, 140 mg, 0.45 mmol) in DMSO (3 mL) were added t-BuOK (152 mg, 1.36 mmol) and cis-cyclohexane-1,2-diol (158 mg, 1.36 mmol) and the reaction mixture was stirred at 90° C. for 1 hour. After completion, it was poured into ice cooled saturated solution of NH₄Cl (15 mL) followed by extraction with ethyl acetate (20 mL×3). The combined organic layers were washed with brine (10 mL), dried over Na₂SO₄, concentrated under reduced pressure to yield a residue, which was separated using silica gel column chromatography to give cis-4-((4-((2-hydroxycyclohexyl)oxy)-5-(trifluoromethyl)pyrimidin-2-yl)amino)benzenesulfonamide (2) in a racemic form, which was further separated by Chiral SFC to give:

Enantiomer 1 (2a, 97.9% ee); Retention time: 3.93 min. LC-MS (ESI): m/z 390.1 [M+H]⁺; ¹H NMR (400 MHz, DMSO-d₆) δ 10.60 (s, 1H), 8.73 (s, 1H), 7.86 (d, J=9.0 Hz, 2H), 7.77 (d, J=8.9 Hz, 2H), 7.26 (s, 2H), 5.33 (s, 1H), 4.85 (d, J=4.7 Hz, 1H), 3.90 (s, 1H), 1.95 (d, J=4.1 Hz, 1H), 1.75-1.53 (m, 5H), 1.34 (m, 2H).

Enantiomer 2 (2b, 99% ee); Retention time: 4.76 min; LC-MS (ESI): m/z 390.0 [M+H]⁺.

Analytical method: Column: ChiralCel OD, 250×4.6 mm I.D., 5 μm; Mobile phase: A for CO₂ and B for MeOH (0.05% DEA); Gradient: 8 min @B 40%; Flow rate: 2.0 mL/min; Back pressure: 100 bar, Column temperature: 35° C.

SFC Method: Instrument: Waters Thar 80 preparative SFC; Column: ChiralCel OD, 250×21.2 mm I.D., 5 μm; Mobile phase: A for CO₂ and B for MeOH+0.1% NH₁₃H₂O; Gradient: B 40%; Flow rate: 50 mL/min; Back pressure: 100 bar; Column temperature: 35° C.

### Illustration 3. Synthesis of cis-4-((2-hydroxy-2-methylcyclopentyl)oxy)-2-((1-(methylsulfonyl)piperidin-4-yl)amino)pyrimidine-5-carbonitrile (3)

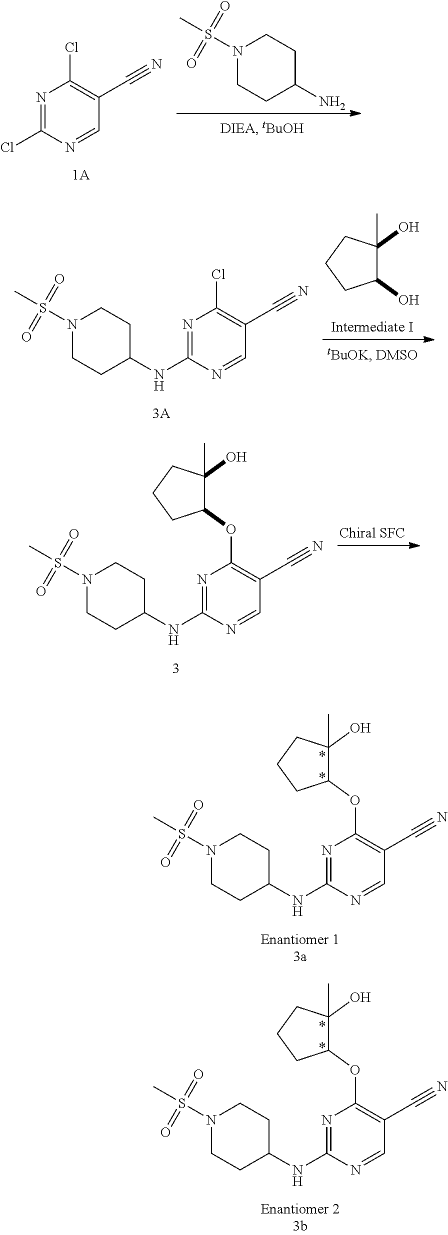

To a solution of 2,4-dichloropyrimidine-5-carbonitrile (1A, 400 mg, 2.30 mmol) in t-BuOH (100 mL) were added 1-(methylsulfonyl)piperidin-4-amine (410 mg, 2.23 mmol) and DIEA (900 mg, 6.89 mmol) and the reaction mixture was stirred at 85° C. for 2 hours. After completion, the reaction mixture was concentrated under reduced pressure and the residue was triturated in dichloromethane (20 mL). The precipitate was collected and dried to afford the 4-chloro-2-((1-(methylsulfonyl)piperidin-4-yl)amino)pyrimidine-5-carbonitrile (3A, 400 mg, 37%) as a white solid. LC-MS (ESI): m/z 316.1 [M+H]⁺.

To a mixture of 4-chloro-2-[(1-methanesulfonylpiperidin-4-yl)amino]pyrimidine-5-carbonitrile (3A, 40 mg, 0.06 mmol) and cis-1-methylcyclopentane-1,2-diol (Intermediate I, 11 mg, 0.10 mmol) in DMSO (1 mL) was added t-BuOK (18 mg, 0.16 mmol). The reaction mixture was stirred at 55° C. for 1.5 hours. After completion, the resulting mixture was poured into ice water (10 mL) and extracted with ethyl acetate (10 mL×3). The combined organic layers were washed with brine (10 mL), dried over Na₂SO₄, concentrated under reduced pressure. The residue was separated using silica gel column chromatography eluted with petroleum ether/ethyl acetate (from 1:0 to 1:2) to give cis-4-((2-hydroxy-2-methylcyclopentyl)oxy)-2-((1-(methylsulfonyl)piperidin-4-yl)amino)pyrimidine-5-carbonitrile (3) in a racemic form, which was further separated by Chiral SFC to give:

Enantiomer 1 (3a, 100% ee); Retention time: 3.16 min. LC-MS (ESI): m/z 396.2 [M+H]⁺;

Enantiomer 2 (3b, 100% ee); Retention time: 3.67 min. LC-MS (ESI): m/z 396.2 [M+H]⁺. ¹H NMR (400 MHz, DMSO-d₆) (tautomer ratio approximately 1:1) δ 8.51 & 8.44 (s, 1H), 8.24 & 8.05 (d, J=8.0 Hz, 1H), 5.14-5.09 (m, 1H), 4.47 (d, J=6.5 Hz, 1H), 3.94-3.82 (m, 1H), 3.56-3.33 (m, 2H), 2.91-2.81 (m, 5H), 2.12-2.05 (m, 1H), 2.00-1.87 (m, 2H), 1.82-1.70 (m, 3H), 1.62-1.51 (m, 4H), 1.22 & 1.20 (s, 3H).

Analytical method: Column: ChiralPak AD, 250×4.6 mm I.D., 5 μm; Mobile phase: A for CO₂ and B for MeOH (0.05% DEA); Gradient: 8 min @B 30%; Flow rate: 2.0 mL/min; Back pressure: 100 bar; Column temperature: 35° C.

SFC Method: Instrument: MG II preparative SFC (SFC-14); Column: ChiralPak AS, 250×30 mm I.D., 10 μm; Mobile phase: A for CO₂ and B for Ethanol; Gradient: B 30%; Flow rate: 70 mL/min; Back pressure: 100 bar; Wavelength: 220 nm; Cycle time: −4 min; Column temperature: 38° C.

### Illustration 4. Synthesis of cis-4-((2-hydroxy-2-methylcyclopentyl)oxy)-2-((1-(methylsulfonyl)piperidin-4-yl)amino)pyrimidine-5-carbonitrile (4)

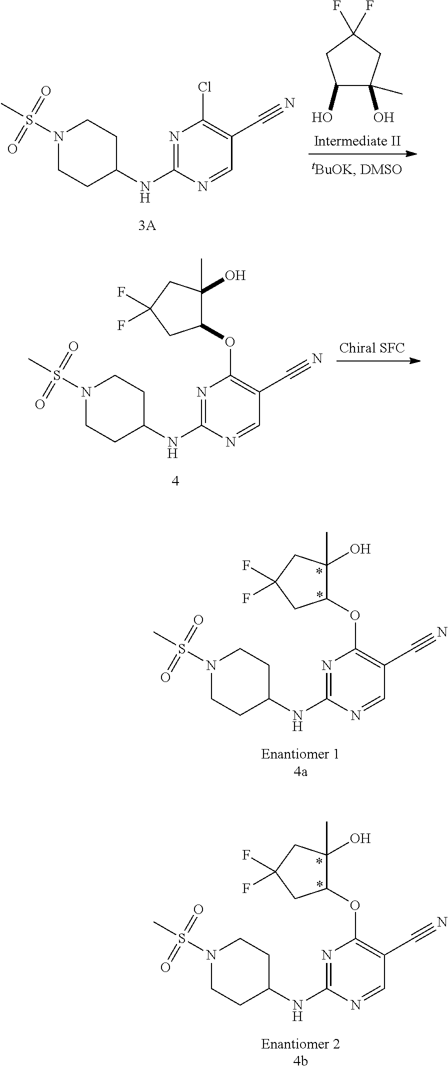

A mixture of 4-chloro-2-((1-(methylsulfonyl)piperidin-4-yl)amino)pyrimidine-5-carbonitrile (3A, 60 mg, 0.19 mmol), cis-4,4-difluoro-1-methylcyclopentane-1,2-diol (Intermediate II, 35 mg, 0.23 mmol) and t-BuOK (43 mg, 0.38 mmol) in DMSO (1 mL) was stirred at 50° C. for 30 mins. The resulting mixture was adjusted to pH 7 with formic acid, and then separated using prep-HPLC to give cis-4-((4,4-difluoro-2-hydroxy-2-methylcyclopentyl)oxy)-2-((1-(methylsulfonyl)piperidin-4-yl)amino)pyrimidine-5-carbonitrile (4, 47 mg, 57%) in a racemic form, which was further separated by Chiral SFC to give:

Enantiomer 1 (4a, 96.3% ee); Retention time: 1.22 min. LC-MS (ESI): m/z 432.2 [M+H]⁺. ¹H NMR (400 MHz, DMSO-d₆) (tautomer ratio approximately 1:1) δ 8.55 & 8.49 (s, 1H), 8.25 & 8.16 (d, J=8.0 Hz, 1H), 5.36-5.22 (m, 1H), 5.13 & 5.12 (s, 1H), 3.99-3.80 (m, 1H), 3.56-3.53 (m, 2H), 2.88-2.82 (m, 6H), 2.44-2.20 (m, 3H), 1.98-1.81 (m, 2H), 1.62-1.52 (m, 2H), 1.31 & 1.30 (s, 3H).

Enantiomer 2 (4b, 95.5% ee); Retention time: 1.45 min. LC-MS (ESI): m/z 432.2 [M+H]⁺.

Analytical method: Column: Chiralpak AS-3, 150×4.6 mm I.D., 3 μm; Mobile phase: 25% of Ethanol (0.05% DEA) in CO₂; Flow rate: 2.5 mL/min; Column temperature: 35° C.

SFC Method: Instrument: MG II preparative SFC (SFC-14); Column: ChiralPak AS, 250×30 mm I.D., 10 μm; Mobile phase: A for CO₂ and B for Isopropanol; Gradient: B 25%; Flow rate: 70 mL/min; Back pressure: 100 bar; Column temperature: 38° C.

### Illustration 5. Synthesis of cis-4-((2-hydroxy-2-methylcyclopentyl)oxy)-2-((1-(methylsulfonyl)piperidin-4-yl)amino)pyrimidine-5-carbonitrile (5)

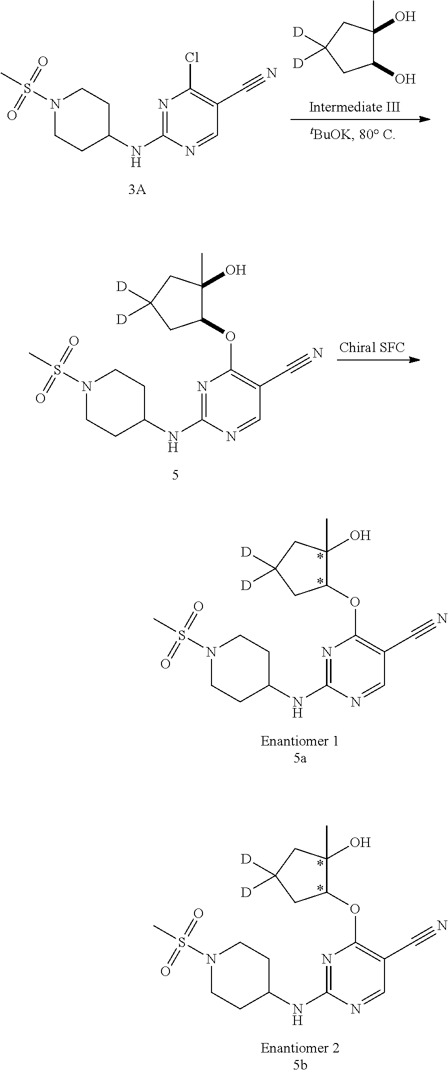

A mixture of 4-chloro-2-((1-(methylsulfonyl)piperidin-4-yl)amino)pyrimidine-5-carbonitrile (3A, 50 mg, 0.16 mmol), cis-1-methylcyclopentane-4,4-d₂-1,2-diol (Intermediate III, 21 mg, 0.17 mmol) and t-BuOK (36 mg, 0.32 mmol) in DMSO (1 mL) was stirred at 80° C. for 30 mins. The reaction mixture was then adjusted to pH 7 with formic acid. The mixture was separated using prep-HPLC to afford cis-4-((2-hydroxy-2-methylcyclopentyl-4,4-d2)oxy)-2-((1-(methylsulfonyl)piperidin-4-yl)amino)pyrimidine-5-carbonitrile (5, 31 mg, 49%) in a racemic form, which was further separated by Chiral SFC to give:

Enantiomer 1 (5a, 100% ee); Retention time: 3.22 min. LC-MS (ESI): m/z 398.2 [M+H]⁺. ¹H NMR (400 MHz, DMSO-d₆) (tautomer ratio approximately 1:1) δ 8.51 & 8.44 (s, 1H), 8.24 & 8.05 (d, J=8.0 Hz, 1H), 5.14-5.17 (m, 1H), 4.48 & 4.47 (s, 1H), 3.94-3.82 (m, 1H), 3.55-3.51 (m, 2H), 2.89-2.81 (m, 5H), 2.08-1.98 (m, 1H), 1.95-1.83 (m, 2H), 1.80-1.64 (m, 2H), 1.60-1.52 (m, 3H), 1.21 & 1.19 (s, 3H).

Enantiomer 2 (5b, 100% ee); Retention time: 3.74 min. LC-MS (ESI): m/z 398.2 [M+H]⁺.

Analytical method: Column: ChiralPak IH, 100×4.6 mm I.D., 5 m; Mobile phase: A for CO₂ and B for methanol (0.05% DEA); Gradient: 0.0 min-1.0 min @ 10% B, 1.0 min-4.5 min gradient (10-40% B), 4.5 min-7.0 min @ 40% B, 7.0 min-8.0 min @ 10% B; Flow rate: 2.5 mL/min; Column temperature: 40° C.

SFC Method: Instrument: IMADZU PREP SOLUTION SFC; Column: ChiralPAK IH, 250×21.2 mm I.D., 5 m; Mobile phase: A for CO₂ and B for MEOH+0.1% NH₃·H₂O; Gradient: B 40%; Flow rate: 40 mL/min; Back pressure: 100 bar; Column temperature: 35° C.

### Illustration 6. Synthesis of 2-((3-fluoro-1-(methylsulfonyl)piperidin-4-yl)amino)-4-((2-hydroxy-2-methylcyclopentyl)oxy)pyrimidine-5-carbonitrile (6)

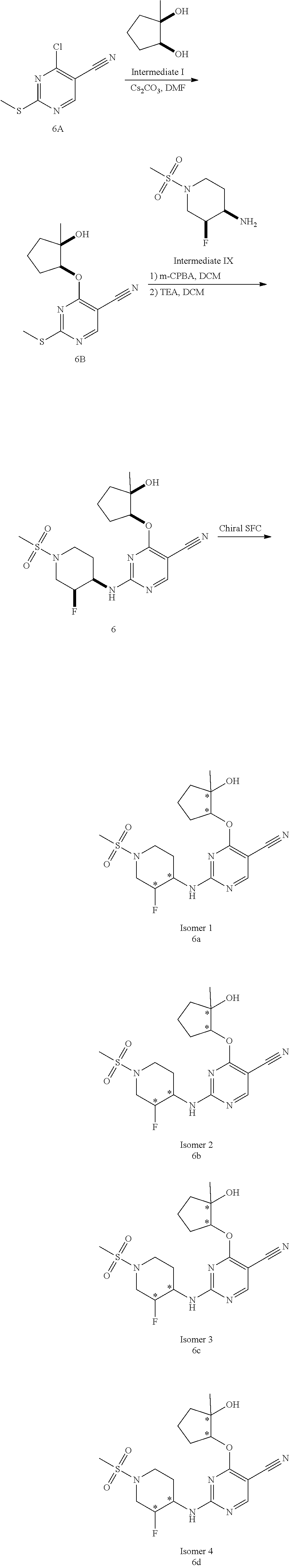

To a mixture of 4-chloro-2-(methylsulfanyl)pyrimidine-5-carbonitrile (6A, 1.00 g, 5.40 mmol) and cis-1-methylcyclopentane-1,2-diol (Intermediate I, 0.75 g, 6.50 mmol) in DMF (10 mL) was added Cs₂CO₃ (3.51 g, 10.8 mmol). The reaction mixture was stirred at room temperature under N₂ for 1 hour. After completion, the reaction mixture was diluted with H₂O (50 mL) and extracted with EtOAc (50 mL×2). The combined organic layers were washed with brine (20 mL), dried over Na₂SO₄, and concentrated under reduced pressure. The resulting residue was separated using silica gel column chromatography to afford cis-4-((2-hydroxy-2-methylcyclopentyl)oxy)-2-(methylthio)pyrimidine-5-carbonitrile (6B, 1.30 g, 91%) as an oil. LC-MS (ESI): m/z 266.2 [M+H]⁺.

To a solution of cis-4-((2-hydroxy-2-methylcyclopentyl)oxy)-2-(methylthio)pyrimidine-5-carbonitrile (6B, 800 mg, 3.00 mmol) in dichloromethane (8 mL) was added m-CPBA (1.04 g, 6.00 mmol) at 0° C. The reaction mixture was stirred at room temperature under N₂ for 2 hours. Then TFA salt of cis-3-fluoro-1-(methylsulfonyl)piperidin-4-amine (Intermediate IX, 643 mg, 2.07 mmol) and triethyl amine (1.27 g, 12.6 mmol) were added at 0° C. The reaction mixture was stirred at room temperature under N₂ for 15 minutes. After completion, the reaction mixture was diluted with H₂O (30 mL) and extracted with dichloromethane (50 mL×2). The combined organic layers were washed with brine (30 mL), dried over Na₂SO₄, and concentrated under reduced pressure. The resulting residue was separated using silica gel column chromatography and prep-HPLC to give cis-2-((3-fluoro-1-(methylsulfonyl)piperidin-4-yl)amino)-4-((2-hydroxy-2-methylcyclopentyl)oxy)pyrimidine-5-carbonitrile (6) in astereoisomeric mixture form, which was further separated by Chiral SFC to give:

Isomer 1 (6a, 100% ee); Retention time: 3.45 min. LC-MS (ESI): m/z 414.2 [M+H]⁺; ¹H NMR (400 MHz, CD₃OD) (tautomer ratio=1:1) δ 8.40 & 8.36 (s, 1H), 5.28-5.19 (m, 1H), 5.00-4.80 (m, 1H), 4.30-4.03 (m, 2H), 3.89-3.85 (m, 1H), 3.30-3.00 (m, 2H), 2.93 & 2.91 (s, 3H), 2.25-2.15 (m, 1H), 2.10-1.95 (m, 1H), 1.95-1.80 (m, 4H), 1.75-1.60 (m, 2H), 1.30 (s, 3H).

Isomer 2 (6b, 99.6% ee); Retention time: 3.58 min. LC-MS (ESI): m/z 414.2 [M+H]⁺.

Isomer 3 (6c, 96.2% ee); Retention time: 4.23 min. LC-MS (ESI): m/z 414.2 [M+H]⁺.

Isomer 4 (6d, 100% ee); Retention time: 4.41 min. LC-MS (ESI): m/z 414.2 [M+H]⁺.

Analytical method: Column: Chiralpak AS-3, 150×4.6 mm I.D., 3 μm; Mobile phase: A: CO₂B: ethanol (0.05% DEA); Gradient: from 5% to 40% of B in 5 min and hold 40% for 2.5 min, then 5% of B for 2.5 min; Flow rate: 2.5 mL/min; Column temperature: 35° C.

SFC Method: Instrument: Waters Thar 80 preparative SFC; Column: Chiralpak AS, 250×21.2 mm I.D., 5 μm; Mobile phase: A for CO₂ and B for MeOH+0.1% NH₃·H₂O; Gradient: B 40%; Flow rate: 40 mL/min; Back pressure: 100 bar; Column temperature: 35° C.

### Illustration 7. Synthesis of 4-((4,4-difluoro-2-hydroxy-2-methylcyclohexyl)oxy)-2-((1-((1-methyl-1H-pyrazol-4-yl)sulfonyl)piperidin-4-yl)amino)pyrimidine-5-carbonitrile (7)

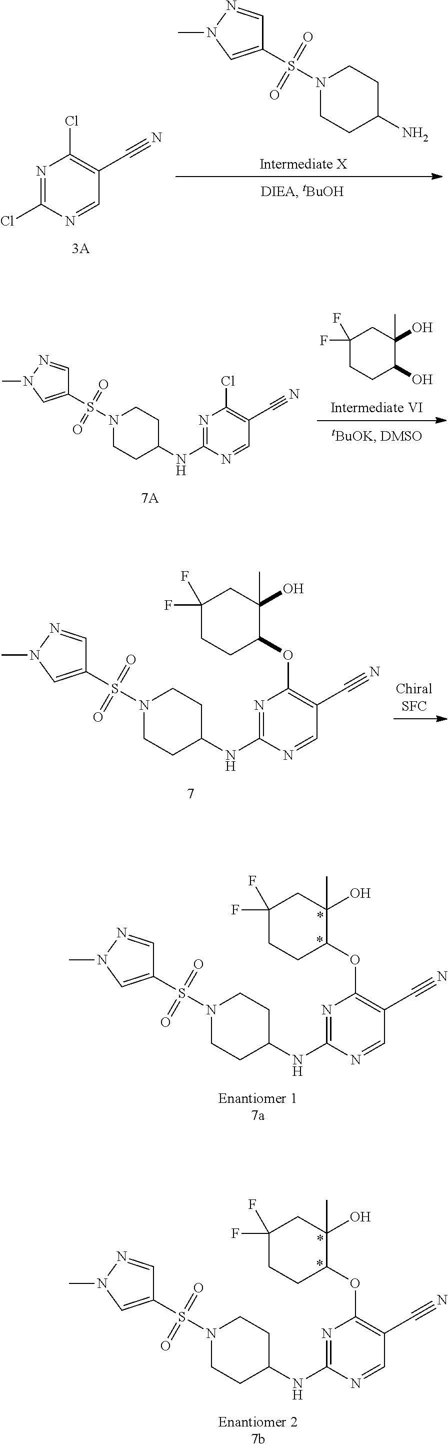

To a mixture of hydrochloride salt of 1-((1-methyl-1H-pyrazol-4-yl)sulfonyl)piperidin-4-amine (Intermediate X, 1.30 g, 4.63 mmol) and 2,4-dichloropyrimidine-5-carbonitrile (3A, 1.20 g, 6.90 mmol) in t-BuOH (10 mL) was added diisopropylethyl amine (3 mL, 16.0 mmol). The reaction mixture was stirred at 50° C. under N₂ for 0.5 hour. After completion, the reaction mixture was diluted with H₂O (30 mL) and extracted with ethyl acetate (30 mL×3). The combined organic layers were washed with brine (20 mL), dried over Na₂SO₄, and concentrated under reduced pressure. The residue was separated using silica gel column chromatography and prep-HPLC to afford 4-chloro-2-((1-((1-methyl-1H-pyrazol-4-yl)sulfonyl)piperidin-4-yl)amino)pyrimidine-5-carbonitrile (7A, 800 mg, 45%) as a white solid. LC-MS (ESI): m/z 382.1 [M+H]⁺.

To a mixture of 4-chloro-2-((1-((1-methyl-1H-pyrazol-4-yl)sulfonyl)piperidin-4-yl)amino)pyrimidine-5-carbonitrile (7A, 200 mg, 0.52 mmol) and cis-5,5-difluoro-1-methylcyclohexane-1,2-diol (Intermediate VI, 131 mg, 0.79 mmol) in DMSO (2 mL) was added t-BuOK (176 mg, 1.57 mmol). The reaction mixture was stirred at 50° C. under N₂ for 2 hours. After completion, the mixture was diluted with H₂O (20 mL) and extracted with ethyl acetate (30 mL×3). The combined organic layers were washed with brine (20 mL), dried over Na₂SO₄, and concentrated under reduced pressure. The residue was separated using silica gel column chromatography and prep-HPLC to give cis-4-((4,4-difluoro-2-hydroxy-2-methylcyclohexyl)oxy)-2-((1-((1-methyl-1H-pyrazol-4-yl)sulfonyl)piperidin-4-yl)amino)pyrimidine-5-carbonitrile (7) in a racemic form, which was further separated by Chiral SFC to give:

Enantiomer 1 (7a, 100% ee); Retention time: 2.73 min. LC-MS (ESI): m/z 512.2 [M+H]⁺; ¹H NMR (400 MHz, DMSO-d₆) (tautomer ratio approximately 1:1) δ 8.50 & 8.46 (s, 1H), 8.34 & 8.32 (s, 1H), 8.27 & 8.12 (d, J=8.0 Hz, 1H), 7.78 & 7.77 (s, 1H), 5.20-5.12 (m, 1H), 4.92 & 4.82 (s, 1H), 3.90 (s, 3H), 3.79-3.78 (m, 1H), 3.55-3.48 (m, 2H), 2.51-2.49 (m, 2H), 2.40-1.89 (m, 8H), 1.64-1.58 (m, 2H), 1.20 & 1.18 (s, 3H).

Enantiomer 2 (7b, 100% ee); Retention time: 4.71 min. LC-MS (ESI): m/z 512.2 [M+H]⁺.

Analytical method: Column: ChiralPak IH, 100×4.6 mm I.D., 5 m; Mobile phase: A for CO₂ and B for methanol (0.05% DEA); Gradient: 8 min @ 20% B; Flow rate: 2.5 mL/min; Column temperature: 40° C.

SFC Method: Instrument: Waters Thar 80 preparative SFC; Column: ChiralPak IH, 250×21.2 mm I.D., 5 μm; Mobile phase: A for CO₂ and B for MeOH+0.1% NH₃·H₂O; Gradient: B 30%; Flow rate: 40 mL/min; Back pressure: 100 bar; Column temperature: 35° C.; Wavelength: 254 nm; Cycle-time: 8 min, Eluted time: 2.3 hr.

### Illustration 8. Synthesis of 2-((1-((1H-pyrazol-4-yl)sulfonyl)piperidin-4-yl)amino)-4-((1-methylcyclopentyl)methoxy)pyrimidine-5-carbonitrile (8)

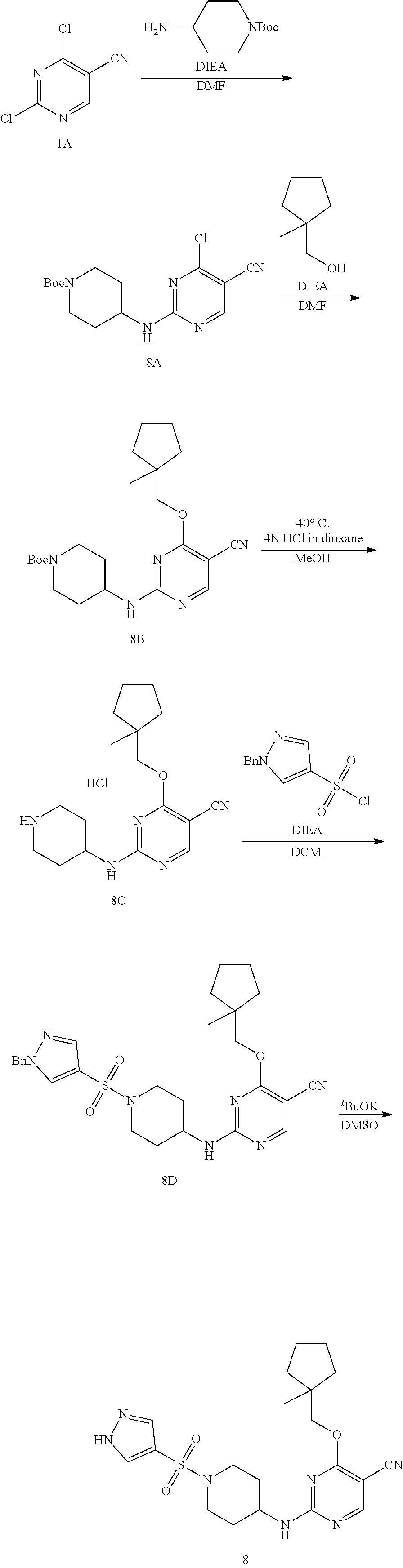

To a mixture of tert-butyl 4-aminopiperidine-1-carboxylate (500 mg, 2.50 mmol) and N,N-diisopropylethyl amine (1.2 mL, 7.50 mmol) in DMF (2 mL) was added a solution of 2,4-dichloropyrimidine-5-carbonitrile (1A, 435 mg, 2.50 mmol) in DMF (1 mL) dropwise at 0° C. Then the mixture was stirred at room temperature for 1 hour. After completion, the mixture was separated using prep-HPLC to give tert-butyl 4-((4-chloro-5-cyanopyrimidin-2-yl)amino)piperidine-1-carboxylate (8A, 420 mg, 50%). LC-MS (ESI): m/z 338.1 [M+H]⁺.

To a mixture of tert-butyl 4-((4-chloro-5-cyanopyrimidin-2-yl)amino)piperidine-1-carboxylate (8A, 216 mg, 0.56 mmol) and (1-methylcyclopentyl)methanol (96 mg, 0.84 mmol) in dry DMSO (2 mL) was added t-BuOK (125 mg, 1.12 mmol). The mixture was stirred at 80° C. for 30 mins. The mixture was then cooled to room temperature, diluted with water (10 mL), and extracted with ethyl acetate (50 mL×2). The organic layers were collected, washed with brine, dried over Na₂SO₄, concentrated under reduced pressure, and the resulting residue was separated using silica gel column chromatography to give tert-butyl 4-((5-cyano-4-((1-methylcyclopentyl)methoxy)pyrimidin-2-yl)amino)piperidine-1-carboxylate (8B, 200 mg, 86%). LC-MS (ESI): m/z 416.3 [M+H]⁺.

To a solution of tert-butyl 4-((5-cyano-4-((1-methylcyclopentyl)methoxy)pyrimidin-2-yl)amino)piperidine-1-carboxylate (8B, 200 mg, 0.48 mmol) in methanol (3 mL) was added HCl (4 M in dioxane, 1 mL). The reaction mixture was stirred at 40° C. for 1 hour, and then concentrated under reduced pressure to give 4-((1-methylcyclopentyl)methoxy)-2-(piperidin-4-ylamino)pyrimidine-5-carbonitrile (8C, 155 mg, 92%) as HCl salt. LC-MS (ESI): m/z 316.2 [M+H]⁺.

To a mixture of 4-((1-methylcyclopentyl)methoxy)-2-(piperidin-4-ylamino)pyrimidine-5-carbonitrile (8C, 100 mg, 0.28 mmol) and N,N-diisopropylethyl amine (140 uL, 0.85 mmol) in dry dichloromethane (2 mL) was added a solution of 1-benzyl-1H-pyrazole-4-sulfonyl chloride (73 mg, 0.28 mmol) in dry dichloromethane (2 mL) carefully at 0° C. The mixture was stirred at room temperature for 1 hour. After completion, the mixture was concentrated under reduced pressure, and the residue was separated using silica gel column chromatography to give 2-((1-((1-benzyl-1H-pyrazol-4-yl)sulfonyl)piperidin-4-yl)amino)-4-((1-methylcyclopentyl)methoxy)pyrimidine-5-carbonitrile (8D, 100 mg, 66%) as a white solid. LC-MS (ESI): m/z 536.2 [M+H]⁺.

To a solution of 2-((1-((1-benzyl-1H-pyrazol-4-yl)sulfonyl)piperidin-4-yl)amino)-4-((1-methylcyclopentyl)methoxy)pyrimidine-5-carbonitrile (8D, 90 mg, 0.17 mmol) in DMSO (1 mL) was added t-BuOK (57 mg, 0.51 mmol), and the mixture was stirred at room temperature overnight. After completion, the mixture was filtered, and the filtrate was separated using prep-HPLC to give 2-((1-((1H-pyrazol-4-yl)sulfonyl)piperidin-4-yl)amino)-4-((1-methylcyclopentyl)methoxy)pyrimidine-5-carbonitrile (8, 24.2 mg, 29%) as a white solid. LC-MS (ESI): m/z 446.2 [M+H]⁺. ¹H NMR (500 MHz, DMSO-d₆) δ 8.49 & 8.45 (s, 1H), 8.30 & 8.16 (d, J=7.6 Hz, 1H), 8.09 (s, 2H), 4.14 (d, J=14.4 Hz, 2H), 3.87-3.64 (m, 1H), 3.51-3.43 (m, 2H), 2.48-2.34 (m, 2H), 1.96-1.87 (m, 2H), 1.67-1.43 (m, 8H), 1.38-1.30 (m, 2H), 1.05 & 1.03 (s, 3H).

### Illustration 9. Synthesis of 4-((5-cyano-4-(piperidin-1-yl) pyrimidin-2-yl) amino) benzenesulfonamide (65)

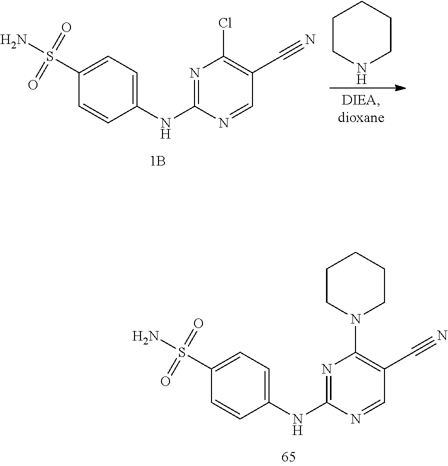

A solution of 4-((4-chloro-5-cyanopyrimidin-2-yl)amino)benzenesulfonamide (1B, 50 mg, 0.16 mmol), piperidine (20 uL, 0.21 mmol) and N,N-diisopropylethyl amine (33 mg, 0.26 mmol) in dioxane (2 mL) was stirred at 60° C. for 2 hours. The reaction mixture was diluted with water (3 mL) and a precipitation was formed. The mixture was filtered and the filter cake was triturated in methanol (5 mL). The solid was collected and dried to give 4-((5-cyano-4-(piperidin-1-yl) pyrimidin-2-yl) amino) benzenesulfonamide (65, 20 mg, 35%). LC-MS (ESI): m/z 359.1 [M+H]⁺. ¹H NMR (400 MHz, DMSO-d₆) δ 10.04 (br s, 1H), 8.34 (s, 1H), 7.72 (d, J=8.8 Hz, 2H), 7.64 (d, J=8.8 Hz, 2H), 7.10 (s, 2H), 3.76-3.74 (m, 4H), 1.56-1.51 (m, 6H).

### Illustration 10. Synthesis of 4-((5-cyano-4-(piperidin-1-yl)pyrimidin-2-yl)amino)-N-(oxetan-3-yl)benzenesulfonamide (66)

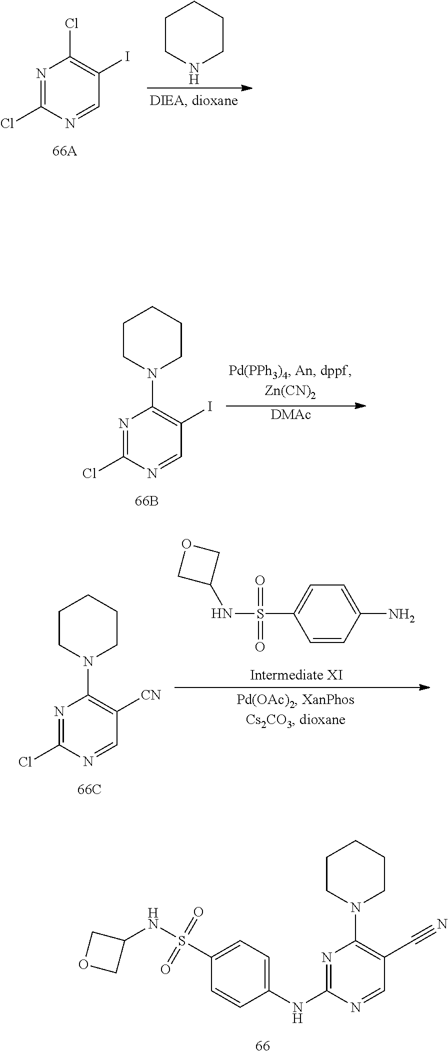

To a solution of 2,4-dichloro-5-iodopyrimidine (66A, 10.0 g, 36.4 mmol) in dioxane (90 mL) were added piperidine (3.41 g, 40.0 mmol) and diisopropylethyl amine (14.1 g, 109 mmol). The reaction mixture was stirred at 25° C. for 15 hours. After completion, the mixture was diluted with H₂O (80 mL) and extracted with EtOAc (80 mL×3). The combined organic layers were washed with brine (60 mL), dried over Na₂SO₄, and concentrated under reduced pressure. The residue was separated using silica gel column chromatography to afford 2-chloro-5-iodo-4-(piperidin-1-yl)pyrimidine (66B, 10.0 g, 85%) as a white solid. LC-MS (ESI): m/z 324.1 [M+H]⁺.

A mixture of 2-chloro-5-iodo-4-(piperidin-1-yl)pyrimidine (66B, 3.00 g, 9.27 mmol), Zn(CN)₂ (2.18 g, 18.5 mmol), Zn (606 mg, 9.27 mmol), Pd(PPh₃)₄ (1.07 g, 0.93 mmol) and 1,1′-bis(diphenylphosphino)ferrocene (dppf) (1.03 g, 1.85 mmol) in dioxane (15 mL) was purged with N₂ before it was subjected to microwave conditions with stirring at 60° C. for 1.5 hours. After completion, the mixture was filtered through a short pad of Celite*, and the filtrate was concentrated under reduced pressure. The resulting residue was separated using flash chromatography to afford 2-chloro-4-(piperidin-1-yl)pyrimidine-5-carbonitrile (66C, 0.9 g, 43%) as a white solid. LC-MS (ESI): m/z 223.1 [M+H]⁺.

To a mixture of 2-chloro-4-(piperidin-1-yl)pyrimidine-5-carbonitrile (66C, 30 mg, 0.13 mmol), 4-amino-N-(oxetan-3-yl)benzenesulfonamide (Intermediate XI, 46 mg, 0.20 mmol), Cs₂CO₃ (132 mg, 0.41 mmol) and XantPhos (16 mg, 0.03 mmol) in dioxane (3 mL) was added Pd(OAc)₂ (5 mg, 0.01 mmol), and the reaction mixture was stirred at 100° C. under N₂ atmosphere overnight. After cooled to room temperature, it was filtered and the filtrate was concentrated under reduced pressure. The residue was separated using silica gel column chromatography to give 4-((5-cyano-4-(piperidin-1-yl)pyrimidin-2-yl)amino)-N-(oxetan-3-yl)benzenesulfonamide (66, 12 mg, 22%). LC-MS (ESI): m/z 415.1 [M+H]⁺. ¹H NMR (400 MHz, DMSO-d₆) δ 10.23 (br s, 1H), 8.48 (s, 1H), 8.37 (br s, 1H), 7.88 (d, J=8.8 Hz, 2H), 7.71 (d, J=8.8 Hz, 2H), 4.49 (t, J=6.8 Hz, 2H), 4.40-4.31 (m, 1H), 4.24 (t, J=6.4 Hz, 2H), 3.90-3.86 (m, 4H), 1.68-1.64 (m, 6H).

### Illustration 11. Synthesis of 4-((5-cyano-4-(4-hydroxyphenyl)pyrimidin-2-yl)amino)benzenesulfonamide (92)

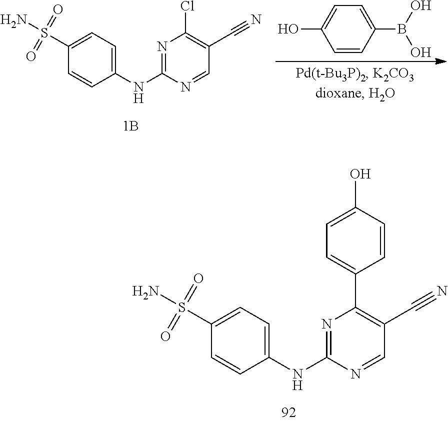 [SMILES: N#Cc1cnc(Nc2ccc(S(N)(=O)=O)cc2)nc1Cl ; OB(O)c1ccc(O)cc1 ; N#Cc1cnc(Nc2ccc(S(N)(=O)=O)cc2)nc1-c1ccc(O)cc1]

To a mixture of 4-((4-chloro-5-cyanopyrimidin-2-yl)amino)benzenesulfonamide (1B, 50 mg, 0.16 mmol) and (4-hydroxyphenyl)boronic acid (27 mg, 0.19 mmol) in dioxane (4 mL) was added a solution of K₂CO₃ (116 mg, 0.83 mmol) in H₂O (1 mL). The reaction mixture was degassed and back-filled with N₂ for 3 times. Pd(t-Bu₃P)₂ (8 mg, 0.02 mmol) was added and the resulting mixture was stirred at 90° C. under N₂ atmosphere for 3 hours. Then the reaction mixture was cooled to room temperature and concentrated under reduced pressure. The resulting residue was separated using prep-HPLC to give 4-((5-cyano-4-(4-hydroxyphenyl)pyrimidin-2-yl)amino)benzenesulfonamide (92, 11 mg, 19%). LC-MS (ESI): m/z 368.1 [M+H]⁺. ¹H NMR (400 MHz, DMSO-d₆) δ 10.73 (br s, 1H), 8.94 (s, 1H), 7.96 (d, J=8.8 Hz, 2H), 7.95 (d, J=8.8 Hz, 2H), 7.79 (d, J=8.8 Hz, 2H), 7.25 (s, 2H), 6.98 (d, J=8.8 Hz, 2H).

### Illustration 12. Synthesis of cis-4-((4-((2-hydroxycyclopentyl)oxy)-5-(trifluoromethyl)pyrimidin-2-yl)amino)benzenesulfonamide (99)

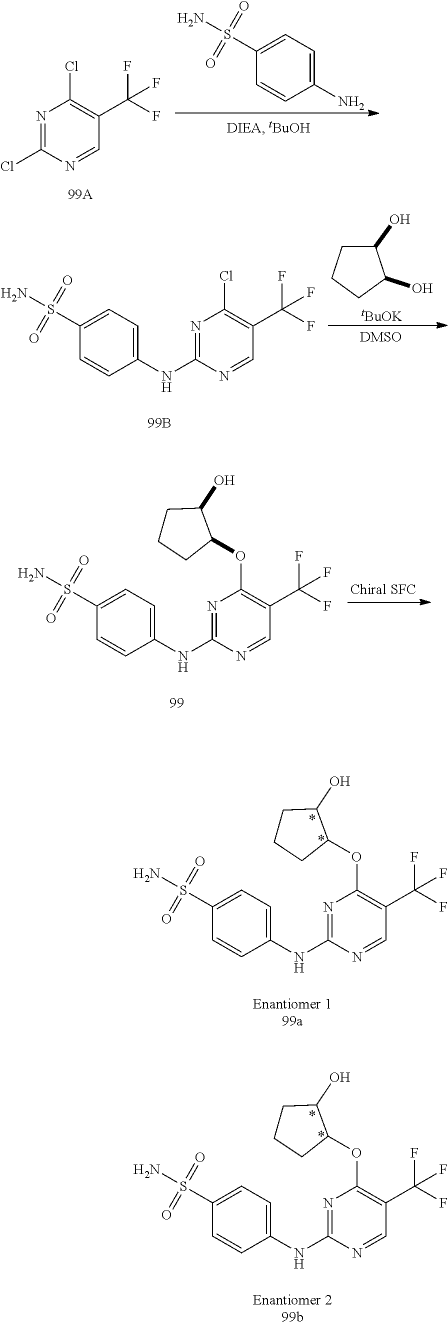

To a solution of 2,4-dichloro-5-(trifluoromethyl)pyrimidine (99A, 2.00 g, 9.22 mmol) in t-BuOH (50 mL) were added 4-aminobenzene-1-sulfonamide (1.59 g, 9.22 mmol) and N,N-diisopropylethyl amine (4.5 mL, 27.7 mmol). The reaction mixture was stirred at 30° C. for 16 hours. After completion, the reaction mixture was concentrated under reduced pressure and the residue triturated in dichloromethane (30 mL). The product was collected and dried to afford 4-((4-chloro-5-(trifluoromethyl)pyrimidin-2-yl)amino)benzenesulfonamide (99B, 0.70 g, 22%) as a white solid. LC-MS (ESI): m/z 353.0 [M+H]⁺. ¹H NMR (400 MHz, DMSO-d₆) δ 11.0 (s, 1H), 8.89 (s, 1H), 7.88 (d, J=8.0 Hz, 2H), 7.80 (d, J=8.0 Hz, 2H), 7.28 (s, 2H).

To a mixture of 4-((4-chloro-5-(trifluoromethyl)pyrimidin-2-yl)amino)benzenesulfonamide (99B, 200 mg, 0.57 mmol) and t-BuOK (127 mg, 1.13 mmol) in DMSO (3 mL) was added cis-cyclopentane-1,2-diol (64 mg, 0.62 mmol). The reaction mixture was stirred at 90° C. for 20 minutes. After completion, the reaction mixture was poured into ice cooled saturated solution of NH₄Cl (15 mL) and extracted with ethyl acetate (20 mL×3). The combined organic layers were washed with brine (10 mL), dried over Na₂SO₄, and concentrated under reduced pressure. The residue was separated using silica gel column chromatography to give cis-4-((4-((2-hydroxycyclopentyl)oxy)-5-(trifluoromethyl)pyrimidin-2-yl)amino)benzenesulfonamide (99) in a racemic form, which was further separated by Chiral SFC to give:

Enantiomer 1 (99a, 92.9% ee); Retention time: 3.19 min. LC-MS (ESI): m/z 419.1[M+H]⁺; 1H NMR (400 MHz, DMSO-d₆) δ 10.40 (s, 1H), 8.56 (s, 1H), 7.90 (d, J=8.0 Hz, 2H), 7.77 (d, J=8.0 Hz, 2H), 7.22 (s, 2H), 5.40-5.32 (m, 1H), 4.71 (d, J=4.0 Hz, 1H), 4.28-4.22 (m, 1H), 2.10-1.92 (m, 1H), 1.92-1.67 (m, 3H), 1.74-1.45 (m, 2H).

Enantiomer 2 (99b, 91.1% ee); Retention time: 4.21 min. LC-MS (ESI): m/z 419.1 [M+H]⁺.

Analytical method: Column: ChiralPak AD, 250×4.6 mm I.D., 5 μm; Mobile phase: A for CO₂ and B for methanol (0.05% DEA; Gradient: 10 min @ 40%; Flow rate: 2.0 mL/min; Back pressure: 100 bar; Column temperature: 35° C.

SFC Method: Instrument: Waters UPC2 analytical SFC; Column: ChiralPAK AD, 250×21.2 mm I.D., 5 m; Mobile phase: A for CO₂ and B for methanol (0.05% DEA); Gradient: 10 min @ 40%; Flow rate: 40 mL/min; Column temperature: 35° C.

### Illustration 13. Synthesis of cis-4-((4-((3-hydroxytetrahydro-2H-pyran-4-yl)oxy)-5-(trifluoromethyl)pyrimidin-2-yl)amino)-N-(methyl-d₃)benzenesulfonamide (100)

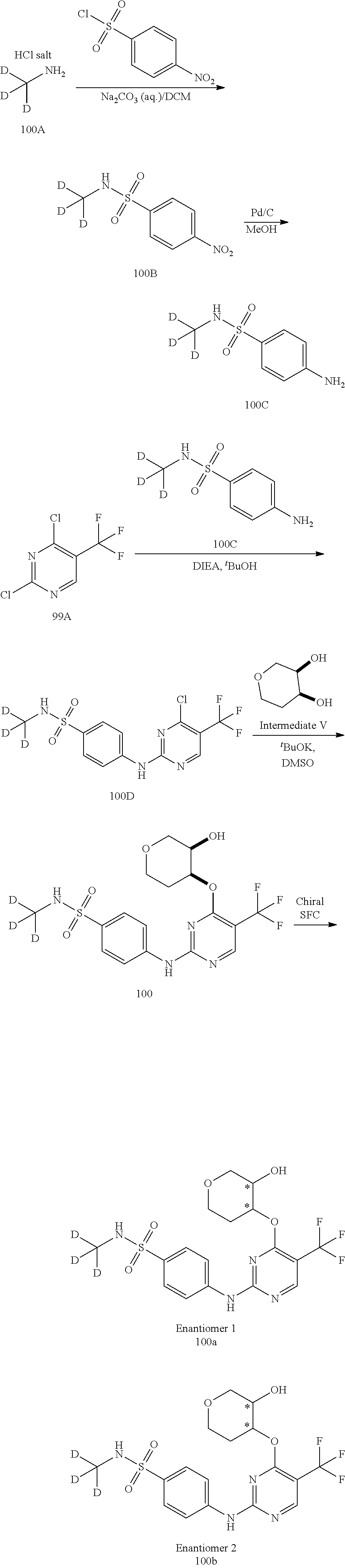

To a mixture of methyl-d₃-amine monohydrochloride (100A, 1.00 g, 14.8 mmol) and 4-nitrobenzenesulfonyl chloride (4.00 g, 17.8 mmol) in dichloromethane (100 mL) was added sodium carbonate (1 M aqueous solution, 45 mL, 45 mmol). The mixture was stirred at room temperature for 2 hours. After completion, the mixture was extracted with dichloromethane (200 mL×2). The extracts were combined, dried over Na₂SO₄, and concentrated under reduced pressure to give N-(methyl-d₃)-4-nitrobenzenesulfonamide (100B, 2.97 g, 91%).

To a solution of N-(methyl-d₃)-4-nitrobenzenesulfonamide (100B, 2.97 g, 13.6 mmol) in methanol (30 mL) was added palladium (10% on carbon, 300 mg). The mixture was stirred at room temperature under one atmosphere of H₂ for 5 hours. The mixture was filtered, and the filtrate was concentrated under reduced pressure to give 4-amino-N-(methyl-d₃)benzenesulfonamide (100C, 2.40 g, 94%) as a white solid. LC-MS (ESI): m/z 190.1 [M+H]⁺.

To a mixture of 4-amino-N-(methyl-d₃)benzenesulfonamide (100C, 2.40 g, 12.7 mmol) and 2,4-dichloro-5-(trifluoromethyl)pyrimidine (99A, 3.30 g, 15.2 mmol) in t-BuOH (60 mL) was added N,N-diisopropylethyl amine (4.92 g, 38.1 mmol). The reaction mixture was stirred at 80° C. under N₂ overnight. After completion, the mixture was concentrated under reduced pressure to yield a residue. The residue was diluted with H₂O (60 mL) and extracted with EtOAc (30 mL×3). The combined organic layers were washed with brine (20 mL), dried over Na₂SO₄ and concentrated under reduced pressure (until 2˜3 mL mixture remained). The mixture was filtered and the filter cake was triturated in dichloromethane (10 mL) to form a yellow solid, which was collected and dried to afford 4-((4-chloro-5-(trifluoromethyl)pyrimidin-2-yl)amino)-N-(methyl-d₃)benzenesulfonamide (100D, 2.12 g, 45%). LC-MS (ESI): m/z 370.1 [M+H]⁺.

To a solution of 4-((4-chloro-5-(trifluoromethyl)pyrimidin-2-yl)amino)-N-(methyl-d₃)benzenesulfonamide (100D, 150 mg, 0.41 mmol) in DMSO (2 mL) were added cis-tetrahydro-2H-pyran-3,4-diol (Intermediate V, 96 mg, 0.81 mmol) and t-BuOK (137 mg, 1.20 mmol). The reaction mixture was stirred at 90° C. for 2 hours. After completion, the reaction mixture was diluted with H₂O (50 mL) and extracted with EtOAc (50 mL×2). The combined organic layers were washed with brine (50 mL), dried over Na₂SO₄, and concentrated under reduced pressure to yield a residue, which was separated using silica gel column chromatography to give cis-4-((4-((3-hydroxytetrahydro-2H-pyran-4-yl)oxy)-5-(trifluoromethyl)pyrimidin-2-yl)amino)-N-(methyl-d₃)benzenesulfonamide (100) in a racemic form, which was further separated by Chiral SFC to give:

Enantiomer 1 (100a, 99% ee); Retention time: 4.20 min. LC-MS (ESI): m/z 452.2 [M+H]⁺; ¹H NMR (400 MHz, DMSO-d₆) δ 10.50 (s, 1H), 8.60 (s, 1H), 7.92 (d, J=8.8 Hz, 2H), 7.73 (d, J=8.7 Hz, 2H), 7.27 (s, 1H), 5.62-5.56 (m, 1H), 5.05 (d, J=4.9 Hz, 1H), 3.94-3.85 (m, 1H), 3.69-3.49 (m, 4H), 2.10-1.81 (m, 2H).

Enantiomer 2 (100b, 98.6% ee); Retention time: 5.41 min. LC-MS (ESI): m/z 452.2 [M+H]⁺;

Analytical method: Column: ChiralCel OD, 250×4.6 mm I.D., 5 μm; Mobile phase: A for CO₂ and B for MeOH (0.05% DEA); Gradient: 8 min @B 30%; Flow rate: 2.0 mL/min; Back pressure: 100 bar; Column temperature: 35° C.

SFC Method: Instrument: Waters Thar 80 preparative SFC; Column: ChiralCel OD, 250×21.2 mm I.D., 5 μm; Mobile phase: A for CO₂ and B for MeOH+0.1% NH₃·H₂O; Gradient: B 25%; Flow rate: 50 mL/min; Back pressure: 100 bar; Column temperature: 35° C.; Wavelength: 256 nm; Cycle-time: 8 min; Eluted time: 1.5 hr.

### Illustration 14. Synthesis of cis-3-fluoro-4-((4-((3-hydroxytetrahydro-2H-pyran-4-yl)oxy)-5-(trifluoromethyl)pyrimidin-2-yl)amino)-N-(methyl-d₃)benzenesulfonamide (101)

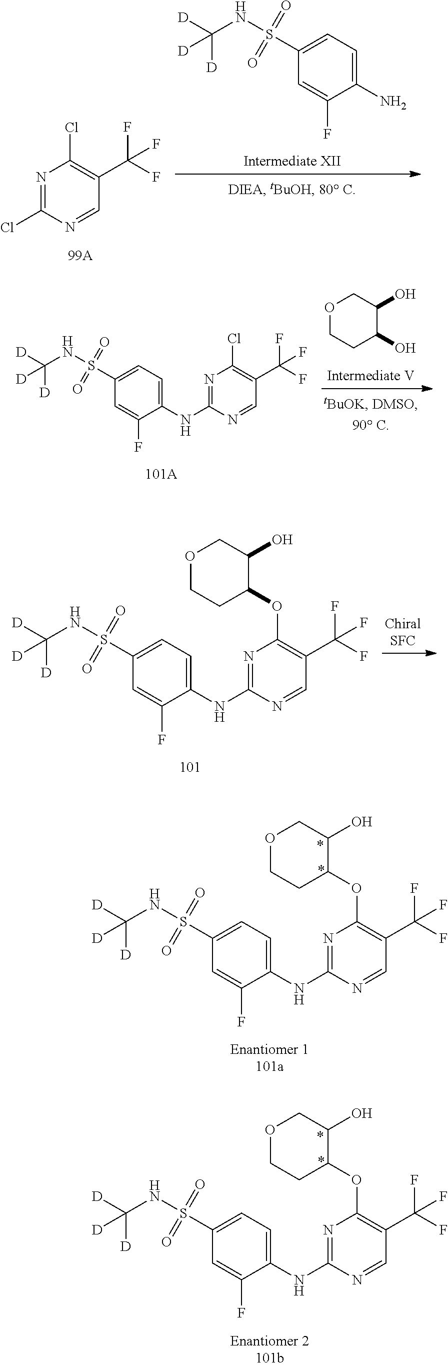

To a mixture of 4-amino-3-fluoro-N-(methyl-d₃)benzenesulfonamide (Intermediate XII, 100 mg, 0.48 mmol) and N,N-diisopropylethylamine (0.17 mL, 0.97 mmol) in t-BuOH (1 mL) was added dropwise a solution of 2,4-dichloro-5-(trifluoromethyl)pyrimidine (99A, 98 uL, 0.72 mmol) in t-BuOH (0.1 mL) at 80° C. The mixture was stirred at 80° C. for 2 hours. Then the second batch of 2,4-dichloro-5-(trifluoromethyl)pyrimidine (99A, 98 uL, 0.72 mmol) in t-BuOH (0.1 mL) was added dropwise at 80° C. The mixture was stirred at 80° C. for another 12 hours. After completion, the mixture was separated using prep-HPLC to give 4-((4-chloro-5-(trifluoromethyl)pyrimidin-2-yl)amino)-3-fluoro-N-(methyl-d₃)benzenesulfonamide (101A, 45 mg, 24%) as a yellow solid. ¹H NMR (500 MHz, DMSO-d₆) δ 10.71 (s, 1H), 8.84 (s, 1H), 7.93 (t, J=7.9 Hz, 1H), 7.72-7.62 (m, 2H), 7.53 (s, 1H).

To a mixture of 4-((4-chloro-5-(trifluoromethyl)pyrimidin-2-yl)amino)-3-fluoro-N-(methyl-d₃)benzenesulfonamide (101A, 80 mg, 0.21 mmol) and cis-tetrahydro-2H-pyran-3,4-diol (Intermediate V, 49 mg, 0.42 mmol) in dimethyl sulfoxide (1 mL) was added t-BuOK (70 mg, 0.62 mmol). The mixture was stirred at 90° C. for 1 hour. The reaction mixture was adjusted to pH 7 with formic acid before it was separated using pre-HPLC to give cis-3-fluoro-4-((4-((3-hydroxytetrahydro-2H-pyran-4-yl)oxy)-5-(trifluoromethyl)pyrimidin-2-yl)amino)-N-(methyl-d₃)benzenesulfonamide (101, 27 mg, 28%) in a racemic form, which was further separated by Chiral SFC to give:

Enantiomer 1 (101a, 100% ee); Retention time: 4.73 min. LC-MS (ESI): m/z 470.1 [M+H]⁺; ¹H NMR (400 MHz, DMSO-d₆) (tautomer ratio=1:1) δ 10.05 (s, 1H), 8.55 (s, 1H), 8.03 (t, J=8.4 Hz, 1H), 7.82-7.62 (m, 2H), 7.50 (s, 1H), 5.45-5.41 (m, 1H), 5.02 (d, J=4.9 Hz, 1H), 3.84-3.81 (m, 1H), 3.61-3.54 (m, 4H), 2.01-1.78 (m, 2H).

Enantiomer 2 (101b, 100% ee); Retention time: 5.39 min. LC-MS (ESI): m/z 470.1 [M+H]⁺.

Analytical method: Column: ChiralPak C-IG, 100×4.6 mm I.D., 5 m; Mobile phase: A for CO₂ and B for methanol (0.05% DEA); Gradient: 0.0 min-1.0 min @ 10% B, 1.0 min-4.5 min gradient (10-40% B), 4.5 min-7.0 min @ 40% B, 7.0 min-8.0 min @ 10% B; Flow rate: 2.5 mL/min; Column temperature: 40° C.

SFC Method: Instrument: IMADZU PREP SOLUTION SFC; Column: ChiralPak C-IG, 250×21.2 mm I.D., 5 m; Mobile phase: A for CO₂ and B for MEOH+0.1% N₁₃·H₂O; Gradient: B 40%; Flow rate: 40 mL/min; Back pressure: 100 bar; Column temperature: 35° C.

### Illustration 15. Synthesis of 4-((4-((5-hydroxyoxepan-4-yl)oxy)-5-(trifluoromethyl)pyrimidin-2-yl)amino)-N-(methyl-d₃)benzenesulfonamide (102)

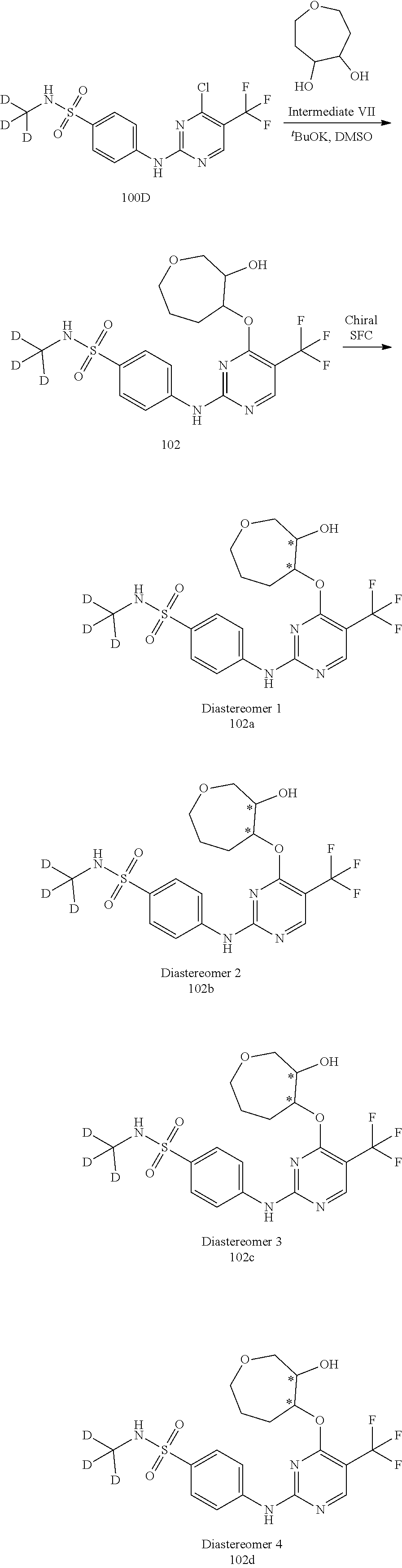

To a mixture of oxepane-4,5-diol (Intermediate VII, 16 mg, 0.12 mmol) and 4-((4-chloro-5-(trifluoromethyl)pyrimidin-2-yl)amino)-N-(methyl-d₃)benzenesulfonamide (100D, 15 mg, 0.04 mmol) in DMSO (1 mL) was added t-BuOK (14 mg, 0.12 mmol) at 0° C. The reaction mixture was stirred at 80° C. for 12 hours. After cooled to room temperature, the solution was poured into saturated solution of NH₄Cl (15 mL), and extracted with ethyl acetate (20 mL×3. The combined organic layers were washed with brine (10 mL dried over Na₂SO₄, and concentrated under reduced pressure. The residue was separated using silica gel column chromatography to give 4-((4-((5-hydroxyoxepan-4-yl)oxy)-5-(trifluoromethyl)pyrimidin-2-yl)amino)-N-(methyl-d₃) benzenesulfonamide (102), which was further separated by Chiral SFC to give:

Diastereomer 1 (102a, 96.7% ee); Retention time: 1.37 min. LC-MS (ESI): m/z 466.3 [M+H]⁺;

Diastereomer 2 (102b, 100% ee); Retention time: 1.45 min. LC-MS (ESI): m/z 466.2 [M+H]⁺; 1H NMR (400 MHz, DMSO-d₆) δ 10.51 (br s, 1H), 8.60 (s, 1H), 7.95 (d, J=8.0 Hz, 2H), 7.74 (d, J=8.0 Hz, 2H), 7.29 (s, 1H), 5.43-5.39 (m, 1H), 5.06 (d, J=4.0 Hz, 1H), 4.02-3.93 (m, 1H), 3.75-3.60 (m, 4H), 2.21-2.15 (m, 1H), 2.00-1.94 (m, 2H), 1.81-1.74 (m, 1H).

Diastereomer 3 (102c, 95.3% ee); Retention time: 1.69 min. LC-MS (ESI): m/z 466.2 [M+H]⁺.

Diastereomer 4 (102d, 100% ee); Retention time: 1.89 min. LC-MS (ESI): m/z 466.2 [M+H]⁺; ¹H NMR (400 MHz, DMSO-d₆) δ 10.51 (br, s, 1H), 8.60 (s, 1H), 7.95 (d, J=8.0 Hz, 2H), 7.74 (d, J=8.0 Hz, 2H), 7.29 (s, 1H), 5.42-5.39 (m, 1H), 5.06 (d, J=4.0 Hz, 1H), 3.97-3.93 (m, 1H), 3.75-3.61 (m, 4H), 2.24-2.15 (m, 1H), 2.00-1.94 (m, 2H), 1.81-1.76 (m, 1H).

Analytical method: Instrument: Waters UPC2 analytical SFC (SFC-H); Column: ChiralPak AD, 150×4.6 mm I.D., 3 μm; Mobile phase: A for CO₂ and B for Ethanol (0.05% DEA); Gradient: B 40%; Flow rate: 2.5 mL/min; Back pressure: 100 bar; Column temperature: 35° C.; Wavelength: 220 nm.

SFC Method:

first round: Instrument: MG II preparative SFC (SFC-14); Column: ChiralPak AD, 250×30 mm I.D., 10 μm; Mobile phase: A for CO₂ and B for Isopropanol (0.1% NH₃·H₂O); Gradient: B 35%. Flow rate: 80 mL/min; Back pressure: 100 bar; Column temperature: 38° C.

second round: Instrument: MG II preparative SFC (SFC-14); Column: ChiralPak AD, 250×30 mm ID., 10 μm; Mobile phase: A for CO₂ and B for Ethanol (0.1% NH₃·H₂O); Gradient: B 35%; Flow rate: 80 mL/min; Back pressure: 100 bar; Column temperature: 38° C.

### Illustration 16. Synthesis of cis-4-((4-((3-hydroxyoxepan-4-yl)oxy)-5-(trifluoromethyl)pyrimidin-2-yl)amino)-N-(methyl-d₃)benzenesulfonamide (103)

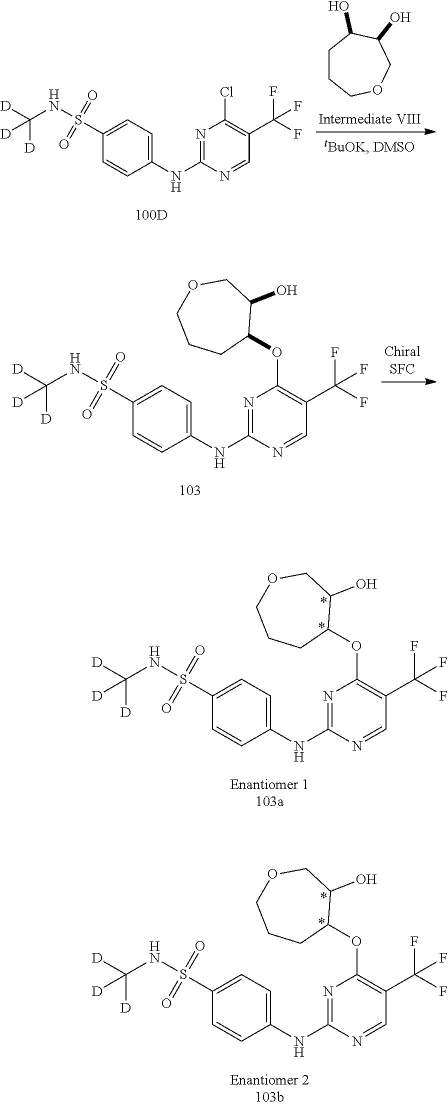

To a mixture of 4-((4-chloro-5-(trifluoromethyl)pyrimidin-2-yl)amino)-N-(methyl-d₃)benzenesulfonamide (100D, 100 mg, 0.27 mmol) and cis-oxepane-3,4-diol (Intermediate VIII, 42 mg, 0.33 mmol) in DMSO (1 mL) was added t-BuOK (91 mg, 0.81 mmol). The mixture was stirred at 80° C. for 2 hours. It was adjusted to pH 7 with formic acid and then separated using prep-HPLC to afford cis-4-((4-((3-hydroxyoxepan-4-yl)oxy)-5-(trifluoromethyl)pyrimidin-2-yl)amino)-N-(methyl-d₃)benzenesulfonamide (103, 27 mg, 28%) in a racemic form, which was further separated by Chiral SFC to give:

Enantiomer 1 (103a, 100% ee); Retention time: 4.67 min. LC-MS (ESI): m/z 466.2 [M+H]⁺. ¹H NMR (400 MHz, DMSO-d₆) δ 10.52 (s, 1H), 8.60 (s, 1H), 7.94 (d, J=8.8 Hz, 2H), 7.73 (d, J=8.8 Hz, 2H), 7.30 (s, 1H), 5.29 (s, 1H), 5.16 (d, J=5.6 Hz, 1H), 3.81-3.72 (m, 3H), 3.65-3.62 (m, 2H), 2.11-1.94 (m, 2H), 1.88-1.78 (m, 2H).

Enantiomer 2 (103b, 95.1% ee); Retention time: 5.08 min. LC-MS (ESI): m/z 466.2 [M+H]⁺.

Analytical method: Column: ChiralPak C-IG, 100×4.6 mm I.D., 5 m; Mobile phase: A for CO₂ and B for methanol (0.05% DEA); Gradient: 0.0 min-1.0 min @ 10% B, 1.0 min-4.5 min gradient (10-40% B), 4.5 min-7.0 min @ 40% B, 7.0 min-8.0 min @ 10% B; Flow rate: 2.5 mL/min; Column temperature: 40° C.

SFC Method: Instrument: IMADZU PREP SOLUTION SFC; Column: ChiralPak C-IG, 250×21.2 mm I.D., 5 um; Mobile phase: A for CO₂ and B for MeOH+0.1% NH₃·H₂O; Gradient: B 40%; Flow rate: 40 mL/min; Back pressure: 100 bar; Column temperature: 35° C.

### Illustration 17. Synthesis of cis-3-methyl-4-((2-((1-(methylsulfonyl)piperidin-4-yl)amino)-5-(trifluoromethyl)pyrimidin-4-yl)oxy)tetrahydrofuran-3-ol (104)

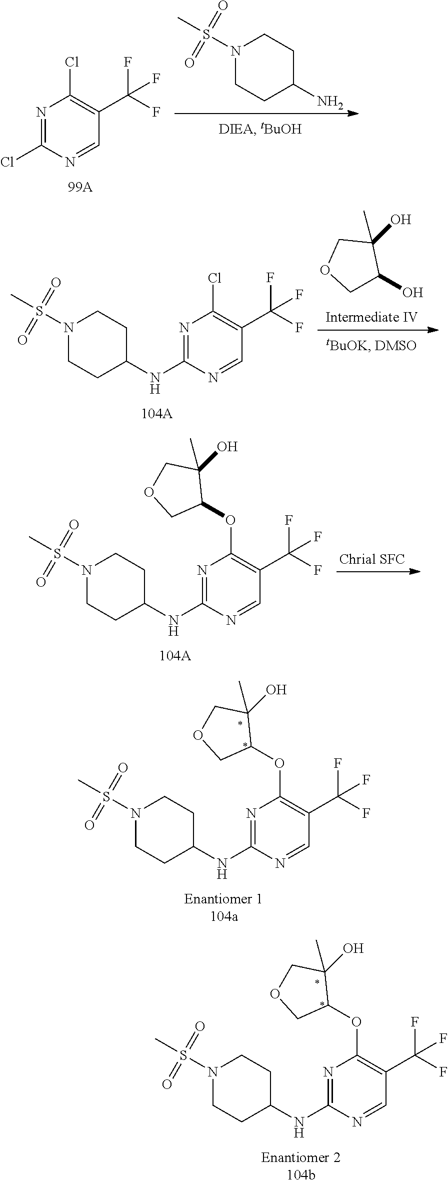

To a mixture of 2,4-dichloro-5-(trifluoromethyl)pyrimidine (99A, 651 mg, 3.00 mmol) and 1-(methylsulfonyl)piperidin-4-amine (534 mg, 3.00 mmol) in t-BuOH (10 mL) was added N,N-diisopropylethyl amine (1.48 mL, 9.00 mmol). The mixture was stirred at 80° C. for 3 hours. After completion, the mixture was concentrated under reduced pressure, and the residue was subjected to prep-HPLC separation to give 4-chloro-N-(1-(methylsulfonyl)piperidin-4-yl)-5-(trifluoromethyl)pyrimidin-2-amine (104A, 367 mg, 30%). LC-MS (ESI): m/z 359.0 [M+H]⁺.

To a mixture of 4-chloro-N-(1-(methylsulfonyl)piperidin-4-yl)-5-(trifluoromethyl)pyrimidin-2-amine (104A, 88 mg, 0.25 mmol) and cis-3-methyltetrahydrofuran-3,4-diol (Intermediate IV, 59 mg, 0.50 mmol) in DMSO (3 mL) was added t-BuOK (83 mg, 0.74 mmol). The reaction mixture was stirred at 90° C. for 30 mins. After completion, the mixture was cooled to room temperature, and then subjected to prep-HPLC separation to afford cis-3-methyl-4-((2-((1-(methylsulfonyl)piperidin-4-yl)amino)-5-(trifluoromethyl)pyrimidin-4-yl)oxy)tetrahydrofuran-3-ol (104, 34 mg, 28%) in a racemic form, which was further separated by Chiral SFC to give:

Enantiomer 1 (104a, 99.6% ee); Retention time: 1.14 min. LC-MS (ESI): m/z 441.2 [M+H]⁺; ¹H NMR (400 MHz, DMSO-d₆) (tautomer ratio approximately 1:1) δ 8.35 & 8.31 (s, 1H), 7.99 & 7.78 (d, J=8.0 Hz, 1H), 5.32-5.22 (m, 1H), 4.83 & 4.81 (s, 1H), 4.22-4.16 (m, 1H), 3.91-3.88 (m, 1H), 3.77-3.75 (m, 1H), 3.57-3.53 (m, 4H), 2.90-2.84 (m, 5H), 2.04-1.91 (m, 2H), 1.57-1.51 (m, 2H), 1.33&1.23 (s, 3H).

Enantiomer 2 (104b, 98.7% ee); Retention time: 1.43 min. LC-MS (ESI): m/z 441.2 [M+H]⁺.

Analytical method: Column: Chiralpak AD-3, 150×4.6 mm I.D., 3 um; Mobile phase: 30% of ethanol (0.05% DEA) in CO₂; Flow rate: 2.5 mL/min; Column temperature: 35° C.

SFC Method: Instrument: MG II preparative SFC(SFC-14); Column: ChiralPak AD, 250×30 mm ID., 10 μm; Mobile phase: A for CO₂ and B for Ethanol (0.1% NH₃·H₂O); Gradient: B 25%; Flow rate: 70 mL/min; Back pressure: 100 bar; Column temperature: 38° C.

### Illustration 18. Synthesis of 4-((5-chloro-4-((2-hydroxycyclopentyl)oxy)pyrimidin-2-yl)amino)-N-(tetrahydro-2H-pyran-4-yl)benzenesulfonamide (130)

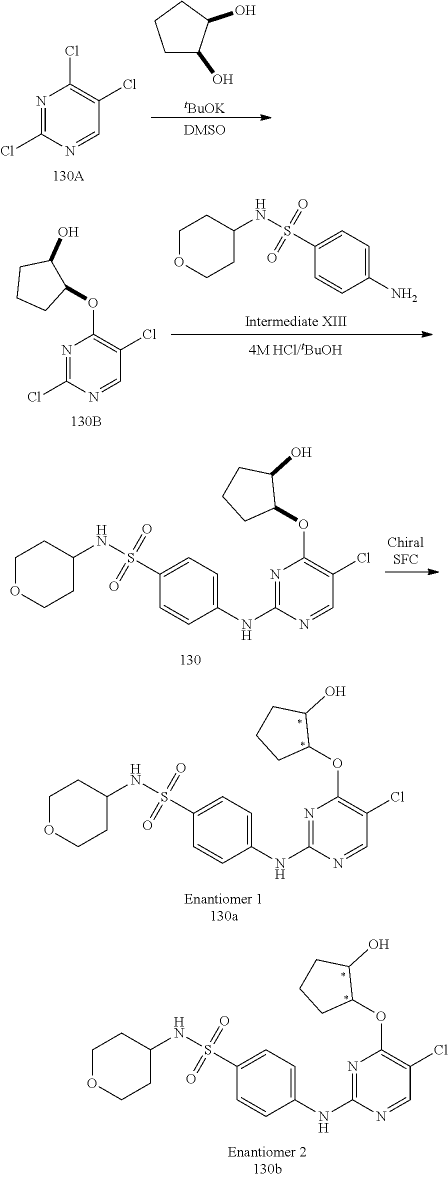

To a mixture of 2,4,5-trichloropyrimidine (130A, 0.68 g, 3.68 mmol) and cis-cyclopentane-1,2-diol (0.39 g, 3.86 mmol) in DMSO (10 mL) was added t-BuOK (0.43 g, 3.86 mmol) at 0° C., and the reaction mixture was stirred at 80° C. for 2 hours. Then the reaction mixture was poured into a cold saturated solution of NH₄Cl (15 mL) and extracted with ethyl acetate (20 mL×3). The combined organic layers were washed with brine (10 mL), dried over Na₂SO₄, and concentrated under reduced pressure. The residue was separated using silica gel column chromatography to give cis-2-((2,5-dichloropyrimidin-4-yl)oxy)cyclopentan-1-ol (130B, 0.38 g, 41%) as a white solid. LC-MS (ESI): m/z 249.0 [M+H]⁺.

To a mixture of 2-[(2,5-dichloropyrimidin-4-yl)oxy]cyclopentan-1-ol (130B, 58 mg, 0.23 mmol) and 4-amino-N-(oxan-4-yl)benzene-1-sulfonamide (63 mg, 0.24 mmol) in anhydrous t-BuOH (1 mL) was added hydrogen chloride (4 M in dioxane, 0.12 mL, 0.47 mmol). The reaction mixture was stirred at 80° C. for 2 hours. After cooled to room temperature, it was poured into saturated aqueous solution of NH₄Cl (15 mL) and extracted with ethyl acetate (20 mL×3). The combined organic layers were washed with brine (10 mL), dried over Na₂SO₄, and concentrated under reduced pressure. The residue was separated using silica gel column chromatography to give cis-4-((5-chloro-4-((2-hydroxycyclopentyl)oxy)pyrimidin-2-yl)amino)-N-(tetrahydro-2H-pyran-4-yl)benzenesulfonamide (130) in a racemic form, which was further separated by Chiral SFC to give:

Enantiomer 1 (130a, 98.3% ee); Retention time: 4.07 min. LC-MS (ESI): m/z 469.2 [M+H]⁺; ¹H NMR (400 MHz, DMSO-d₆) δ 10.06 (br s, 1H), 8.35 (s, 1H), 7.88 (d, J=9.0 Hz, 2H), 7.72 (d, J=8.9 Hz, 2H), 7.59 (d, J=7.0 Hz, 1H), 5.27-5.21 (m, 1H), 4.72 (d, J=4.8 Hz, 1H), 4.29-4.19 (m, 1H), 3.74-3.66 (m, 2H), 3.33-3.20 (m, 3H), 1.89-1.82 (m, 1H), 1.87-1.78 (m, 3H), 1.71-1.61 (m, 1H), 1.59-1.49 (m, 3H), 1.40-1.28 (m, 2H).

Enantiomer 2 (130b, 98.7% ee); Retention time: 5.95 min. LC-MS (ESI): m/z 469.2 [M+H]⁺; ¹H NMR (400 MHz, DMSO-d₆) δ 10.07 (br s, 1H), 8.35 (s, 1H), 7.88 (d, J=8.9 Hz, 2H), 7.72 (d, J=8.9 Hz, 2H), 7.59 (d, J=7.2 Hz, 1H), 5.28-5.23 (m, 1H), 4.72 (d, J=4.8 Hz, 1H), 4.30-4.20 (m, 1H), 3.75-3.67 (m, 2H), 3.24-3.19 (m, 3H), 2.07-2.01 (m, 1H), 1.88-1.78 (m, 3H), 1.70-1.60 (m, 1H), 1.55-1.45 (m, 3H), 1.37-1.29 (m, 2H).

Analytical method: Column: ChiralCel OD, 250×4.6 mm I.D., 5 μm; Mobile phase: A for CO₂ and B for MeOH (0.05% DEA); Gradient: 8 min @B 40%; Flow rate: 2.0 mL/min; Back pressure: 100 bar; Column temperature: 35° C.

SFC Method: Instrument: Waters UPC2 analytical SFC; Column: ChiralPak AD, 250×4.6 mm I.D., 5 μm; Mobile phase: A for CO₂ and B for methanol (0.05% DEA); Gradient: 10 min @ 40%; Flow rate: 2.0 mL/min; Back pressure: 100 bar; Column temperature: 35° C.

### Illustration 19. Synthesis of cis-4-((5-bromo-4-((2-hydroxycyclopentyl)oxy)pyrimidin-2-yl)amino)-N-(3-methyloxetan-3-yl)benzenesulfonamide (131)

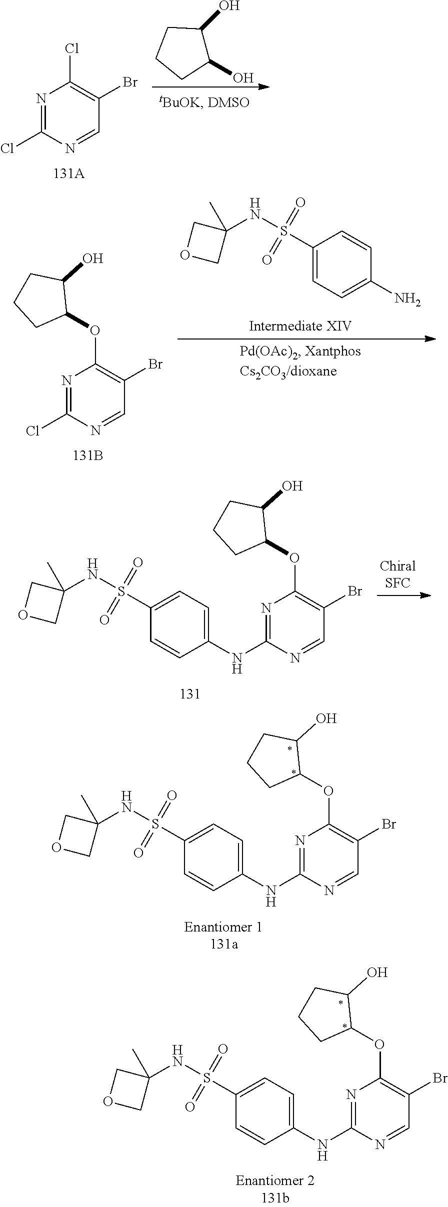

To a mixture of 5-bromo-2,4-dichloropyrimidine (131A, 113 mg, 0.50 mmol) and cis-cyclopentane-1,2-diol (50 mg, 0.50 mmol) in DMSO (2 mL) was added t-BuOK (67 mg, 0.60 mmol) and the mixture was stirred at room temperature for 2 hours. After completion, the mixture was separated using prep-HPLC to afford cis-2-((5-bromo-2-chloropyrimidin-4-yl)oxy)cyclopentan-1-ol (131B, 108 mg, 74%) as a white solid. LC-MS (ESI): m/z 293.0 [M+H]⁺.

To a mixture of cis-2-[(5-bromo-2-chloropyrimidin-4-yl)oxy]cyclopentan-1-ol (131B, 92 mg, 0.31 mmol) and 4-amino-N-(3-methyloxetan-3-yl)benzene-1-sulfonamide (Intermediate XIV, 76 mg, 0.31 mmol) in anhydrous dioxane (1 mL) under nitrogen atmosphere were added Pd(OAc)₂ (4 mg, 0.02 mmol), Xantphos (18 mg, 0.03 mmol) and Cs₂CO₃ (204 mg, 0.63 mmol). The reaction mixture was stirred under nitrogen atmosphere at 100° C. for 4 hours. After cooled to room temperature, the mixture was diluted with ethyl acetate (20 mL), filtered through a celite pad and washed with ethyl acetate (10 mL). The filtrate was concentrated under reduced pressure. The residue was separated using silica gel column chromatography to give cis-4-({5-bromo-4-[(2-hydroxycyclopentyl)oxy]pyrimidin-2-yl}amino)-N-(3-methyloxetan-3-yl)benzene-1-sulfonamide (131) in a racemic form, which was further separated by Chiral SFC to give:

Enantiomer 1 (131a, 94.2% ee); Retention time: 4.39 min. LC-MS (ESI): m/z 499.1 & 501.1 [M+H]*; ¹H NMR (400 MHz, DMSO-d₆) δ 10.10 (br s, 1H), 8.43 (s, 1H), 8.14 (br s, 1H), 7.90 (d, J=8.8 Hz, 2H), 7.72 (d, J=8.8 Hz, 2H), 5.33-5.19 (m, 1H), 4.71 (d, J=4.8 Hz, 1H), 4.53 (d, J=5.6 Hz, 2H), 4.32-4.22 (m, 1H), 4.08 (d, J=6.0 Hz, 2H), 2.11-1.97 (m, 1H), 1.91-1.77 (m, 3H), 1.67-1.55 (m, 2H), 1.43 (s, 3H).

Enantiomer 2 (131b, 93.9% ee); Retention time: 7.54 min. LC-MS (ESI): m/z 499.1 & 501.1 [M+H]⁺.

Analytical method: Column: ChiralCel OD, 250×21.2 mm I.D., 5 m; Mobile phase: A for CO₂ and B for methanol (0.05% DEA); Gradient: 10 min @ 40%; Flow rate: 40 mL/min; Column temperature: 35° C.

SFC Method: Instrument: Waters UPC2 analytical SFC; Column: ChiralPak AD, 250×4.6 mm I.D., 5 μm; Mobile phase: A for CO₂ and B for methanol (0.05% DEA); Gradient: 10 min @ 40%; Flow rate: 2.0 mL/min; Back pressure: 100 bar; Column temperature: 35° C.

### Illustration 20. Synthesis of cis-4-((4-((2-hydroxycyclopentyl)oxy)-5-methylpyrimidin-2-yl)amino)benzenesulfonamide (132)

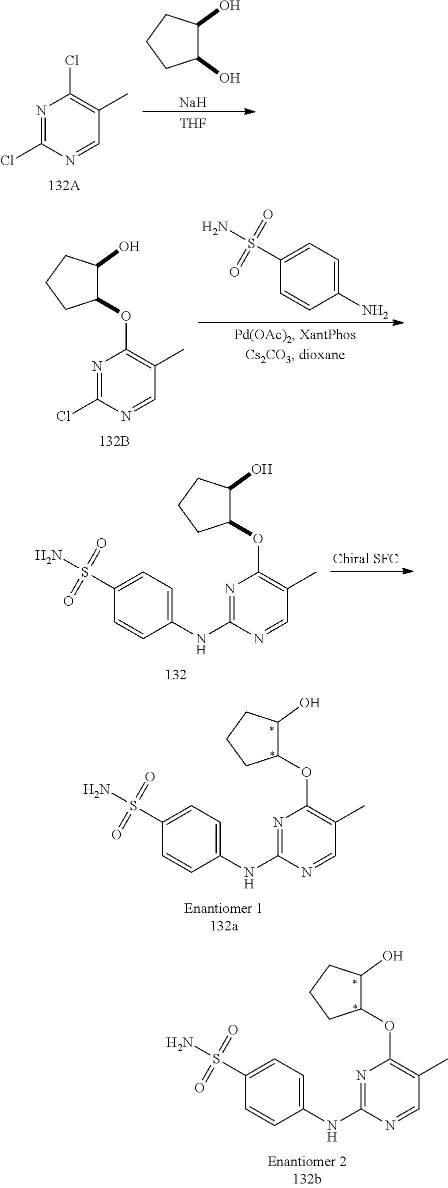

To a solution of cis-cyclopentane-1,2-diol (100 mg, 0.98 mmol) in THE (3 mL) was added NaH (60% in mineral oil, 78 mg, 1.96 mmol,) at 0° C. and the reaction mixture was heated to 40° C. Then a solution of 2,4-dichloro-5-methylpyrimidine (132A, 144 mg, 0.88 mmol) in THE (3 mL) was added dropwise. After completion, the reaction mixture was poured into ice cooled saturated solution of NH₄Cl (15 mL) and extracted with ethyl acetate (10 mL×3). The combined organic layers were washed with brine (10 mL), dried over Na₂SO₄ and concentrated under reduced pressure. Then the residue was separated using silica gel column chromatography to give cis-2-((2-chloro-5-methylpyrimidin-4-yl)oxy)cyclopentan-1-ol (132B, 150 mg, 75%) as a white solid. LC-MS (ESI): m/z 229.0 [M+H]⁺.

A mixture of cis-2-((2-chloro-5-methylpyrimidin-4-yl)oxy]cyclopentan-1-ol (132B, 100 mg, 0.44 mmol), 4-aminobenzene-1-sulfonamide (114 mg, 0.66 mmol), Cs₂CO₃ (427 mg, 1.31 mmol), Pd(OAc)₂ (10 mg, 0.04 mmol) and XantPhos (51 mg, 0.09 mmol) in dioxane (3 mL) was degassed and backfilled with N₂ for three times and then sealed in a tube and stirred at 100° C. under microwave conditions for 1 hour. After completion, the reaction mixture was concentrated under reduced pressure and the residue was separated using silica gel column chromatography to afford cis-4-((4-((2-hydroxycyclopentyl)oxy)-5-methylpyrimidin-2-yl)amino)benzene-1-sulfonamide (132) in a racemic form, which was further separated by Chiral SFC to give:

Enantiomer 1 (132a, 96.1% ee); Retention time: 3.20 min. LC-MS (ESI): m/z 365.1 [M+H]⁺; 1H NMR (400 MHz, DMSO-d₆) δ 9.69 (s, 1H), 8.08 (s, 1H), 7.88 (d, J=8.9 Hz, 2H), 7.69 (d, J=8.8 Hz, 2H), 7.13 (s, 2H), 5.27-5.19 (m, 1H), 4.62 (d, J=5.0 Hz, 1H), 4.26-4.17 (m, 1H), 2.06-1.97 (m, 4H), 1.88-1.75 (m, 3H), 1.72-1.62 (m, 1H), 1.61-1.50 (m, 1H).

Enantiomer 2 (132b, 93.4% ee); Retention time: 3.76 min. LC-MS (ESI): m/z 365.0 [M+H]⁺.

Analytical method: Column: ChiralCel OJ, 250×4.6 mm I.D., 5 μm; Mobile phase: A for CO₂ and B for MeOH (0.05% DEA); Gradient: 8 min @B 40%; Flow rate: 2.0 mL/min; Back pressure: 100 bar; Column temperature: 35° C.

SFC Method: Instrument: Waters Thar 80 preparative SFC; Column: ChiralCel OJ, 250×21.2 mm I.D., 5 μm; Mobile phase: A for CO₂ and B for MeOH+0.1% NH₃·H₂O; Gradient: B 40%; Flow rate: 50 mL/min; Back pressure: 100 bar; Column temperature: 35° C.

### Illustration 21. Synthesis of cis-4-((5-ethyl-4-((2-hydroxycyclopentyl)oxy)pyrimidin-2-yl)amino)benzenesulfonamide (133)

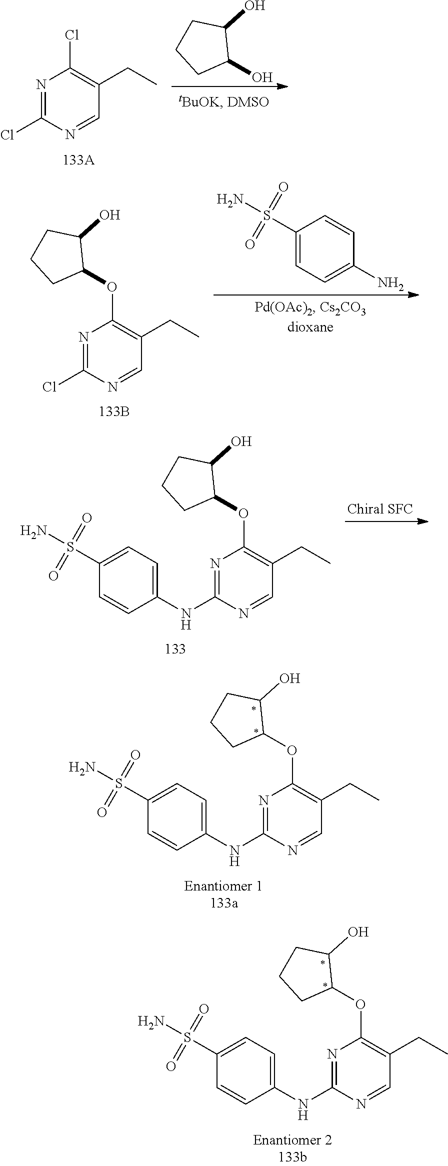

To a mixture of 2,4-dichloro-5-ethylpyrimidine (133A, 2.98 g, 19.6 mmol) and cis-cyclopentane-1,2-diol (2.00 g, 19.6 mmol) in DMSO (60 mL) was added t-BuOK (6.60 g, 58.9 mmol) in portions at 0° C. The reaction mixture was stirred at room temperature for 1 hour. After completion, the reaction mixture was poured into ice cooled saturated solution of NH₄Cl (60 mL) and extracted with ethyl acetate (80 mL×3). The combined organic layers were washed with brine (20 mL), dried over Na₂SO₄, and concentrated under reduced pressure. The resulting residue was separated using silica gel column chromatography to give cis-2-((2-chloro-5-ethylpyrimidin-4-yl)oxy)cyclopentan-1-ol (133B, 1.80 g, 38%) as a white solid. LC-MS (ESI): m/z 243.0 [M+H]⁺.

A mixture of cis-2-((2-chloro-5-ethylpyrimidin-4-yl)oxy)cyclopentan-1-ol (133B, 510 mg, 2.10 mmol), 4-aminobenzenesulfonamide (360 mg, 2.10 mmol), Cs₂CO₃ (2.05 g, 6.30 mmol), Pd(OAc)₂ (140 mg, 0.63 mmol) and Xantphos (370 mg, 0.63 mmol) in dioxane (5 mL) was purged with N₂ before it was subjected to microwave conditions with stirring at 100° C. for 1.5 hours. After completion, the reaction mixture was concentrated under reduced pressure and the residue was separated using silica gel column chromatography to give cis-4-((5-ethyl-4-((2-hydroxycyclopentyl)oxy)pyrimidin-2-yl)amino)benzenesulfonamide (133) in a racemic form, which was further separated by Chiral SFC to give:

Enantiomer 1 (133a, 91.1% ee); Retention time: 4.64 min. LC-MS (ESI): m/z 379.1 [M+H]⁺; 1H NMR (400 MHz, DMSO-d₆) δ 9.71 (s, 1H), 8.08 (s, 1H), 7.90 (d, J=8.8 Hz, 2H), 7.71 (d, J=8.8 Hz, 2H), 7.13 (s, 2H), 5.26 (d, J=4.4 Hz, 1H), 4.65 (d, J=4.4 Hz, 1H), 4.23 (t, J=4.4 Hz, 1H), 2.47-2.42 (m, 2H), 2.05-1.98 (m, 1H), 1.91-1.75 (m, 3H), 1.71-1.55 (m, 2H), 1.16 (t, J=7.2 Hz, 3H).

Enantiomer 2 (133b, 90.5% ee); Retention time: 5.72 min. LC-MS (ESI): m/z 379.1 [M+H]⁺.

Analytical method: Column: ChiralPak IA, 250×4.6 mm I.D., 5 μm; Mobile phase: A for CO₂ and B for MeOH (0.05% DEA); Gradient: 8 min @B 40%; Flow rate: 1.8 mL/min; Back pressure: 100 bar; Column temperature: 35° C.

SFC Method: Instrument: Waters Thar 80 preparative SFC; Column: ChiralCel IA, 250×21.2 mm I.D., 5 μm; Mobile phase: A for CO₂ and B for MeOH+0.1% DEA; Gradient: B 40%; Flow rate: 50 mL/min; Back pressure: 100 bar; Column temperature: 35° C.

### Illustration 22. Synthesis of cis-4-((5-cyclopropyl-4-((2-hydroxycyclopentyl)oxy)pyrimidin-2-yl)amino)-N-isopropylbenzenesulfonamide (134)

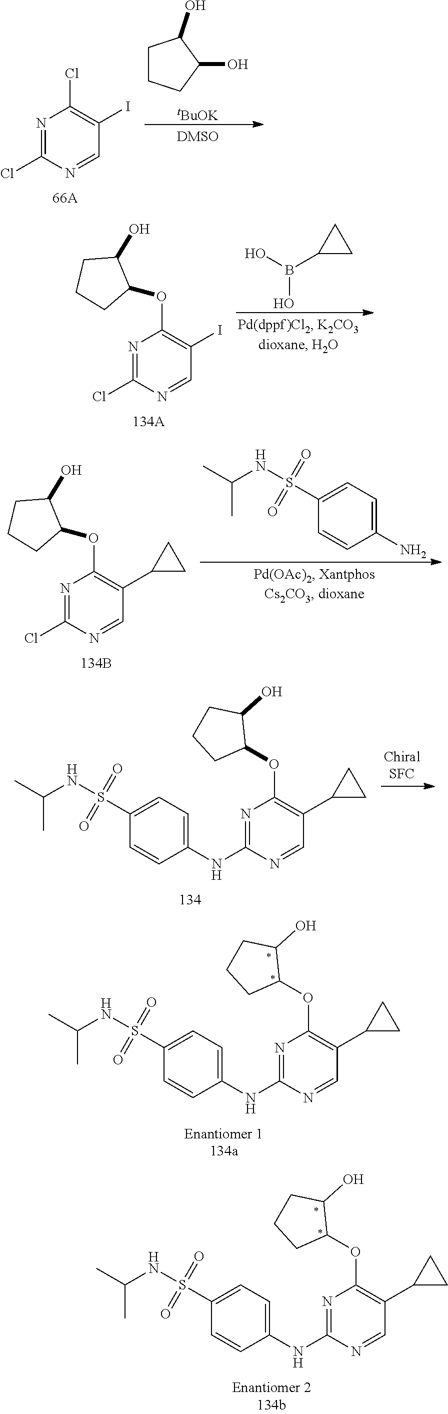

To a mixture of 2,4-dichloro-5-iodopyrimidine (66A, 5.40 g, 19.6 mmol) and cis-cyclopentane-1,2-diol (2.00 g, 19.6 mmol) in DMSO (60 mL) was added t-BuOK (6.60 g, 58.9 mmol) in portions at 0° C. The reaction mixture was stirred at room temperature for 1 hour. After completion, the reaction mixture was poured into ice cooled saturated solution of NH₄Cl (60 mL) and extracted with ethyl acetate (80 mL×3). The combined organic layers were washed with brine (20 mL), dried over Na₂SO₄, and concentrated under reduced pressure. The residue was separated using silica gel column chromatography to give cis-2-((2-chloro-5-iodopyrimidin-4-yl)oxy)cyclopentan-1-ol (134A, 4.5 g, 67%) as a white solid. LC-MS (ESI): m/z 341.0 [M+H]⁺.

A mixture of cis-2-((2-chloro-5-iodopyrimidin-4-yl)oxy)cyclopentan-1-ol (134A, 500 mg, 1.46 mmol), cyclopropylboronic acid (250 mg, 2.92 mmol), K₂CO₃ (607 mg, 4.40 mmol) and Pd(dppf)Cl₂ (107 mg, 0.14 mmol) in 1,4-dioxane (8 mL) and H₂O (2 mL) was degassed and backfilled with N₂ for 3 times. The reaction mixture was stirred at 100° C. under N₂ atmosphere for 5 hours. After completion, the reaction mixture was concentrated under reduced pressure, and the residue was separated using silica gel column chromatography to give cis-2-((2-chloro-5-cyclopropylpyrimidin-4-yl)oxy)cyclopentan-1-ol (134B, 210 mg, 56%) as a white solid. LC-MS (ESI): m/z 255.0 [M+H]⁺.

A mixture of cis-2-((2-chloro-5-cyclopropylpyrimidin-4-yl)oxy)clopentan-1-ol (134B, 130 mg, 0.51 mmol), 4-amino-N-isopropylbenzenesulfonamide (101 mg, 0.51 mmol), CS₂CO₃ (497 mg, 1.53 mmol), Pd(OAc)₂ (34 mg, 0.15 mmol) and Xantphos (88 mg, 0.15 mmol) in dioxane (5 mL) was purged with N₂ before it was subjected to microwave conditions with stirring at 100° C. for 1 hour. After completion, the reaction mixture was concentrated under reduced pressure, and the residue was separated using silica gel column chromatography to give cis-4-((5-cyclopropyl-4-((2-hydroxycyclopentyl)oxy)pyrimidin-2-yl)amino)-N-isopropylbenzenesulfonamide (134) in a racemic form, which was further separated by Chiral SFC to give:

Enantiomer 1 (134a, 97.2% ee); Retention time: 3.23 min. LC-MS (ESI): m/z 433.0 [M+H]⁺; ¹H NMR (400 MHz, DMSO-d₆) δ 9.75 (s, 1H), 7.94 (s, 1H), 7.90 (d, J=8.8 Hz, 2H), 7.67 (d, J=8.8 Hz, 2H), 7.32 (d, J=7.2 Hz, 1H), 5.26-5.22 (m, 1H), 4.65 (d, J=4.8 Hz, 1H), 4.32-4.16 (m, 1H), 3.25-3.17 (m, 1H), 2.10-1.97 (m, 1H), 1.90-1.75 (m, 4H), 1.74-1.64 (m, 1H), 1.63-1.50 (m, 1H), 0.94 (d, J=6.5 Hz, 6H), 0.86-0.76 (m, 3H), 0.74-0.67 (m, 1H).

Enantiomer 2 (134b, 97.6% ee); Retention time: 4.34 min. LC-MS (ESI): m/z 433.1 [M+H]⁺.

Analytical method: Column: ChiralCel OD, 250×4.6 mm I.D., 5 μm; Mobile phase: A for CO₂ and B for EtOH (0.05% DEA); Gradient: 8 min @B 40%; Flow rate: 1.8 mL/min; Back pressure: 100 bar; Column temperature: 35° C.

SFC Method: Instrument: Waters Thar 80 preparative SFC; Column: ChiralCel OD, 250×21.2 mm I.D., 5 μm; Mobile phase: A for CO₂ and B for MeOH+0.1% NH₁₃H₂O; Gradient: B 40%; Flow rate: 50 mL/min; Back pressure: 100 bar; Column temperature: 35° C.

### Illustration 23. Synthesis of cis-4-((5-(1-fluorovinyl)-4-((2-hydroxycyclopentyl)oxy)pyrimidin-2-yl)amino)-N-(methyl-d₃)benzenesulfonamide (135)

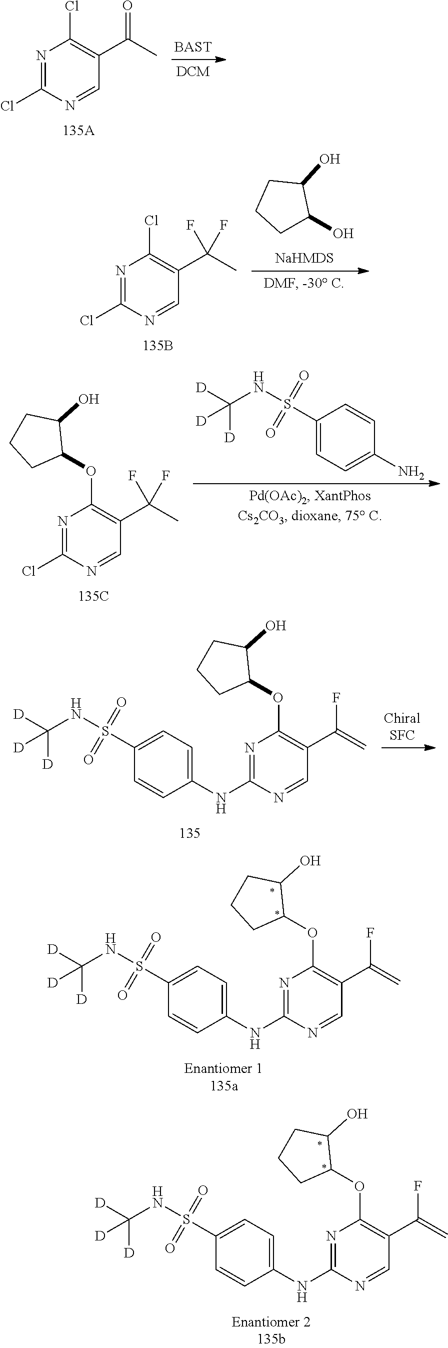

To a solution of 1-(2,4-dichloropyrimidin-5-yl)ethan-1-one (135A, 950 mg, 5.00 mmol) in dichloromethane (20 mL) was added bis(2-methoxyethyl)aminosulfur trifluoride (BAST) (3.32 g, 15.0 mmol) dropwise at 0° C. Then the reaction mixture was stirred at room temperature for 16 hours. After completion, the mixture was poured into ice-water (20 mL) carefully and extracted with dichloromethane (20 mL×3). The organic layer was combined, dried over anhydrous Na₂SO₄, and then evaporated under reduced pressure. The residue was separated using silica gel column chromatography to give 2,4-dichloro-5-(1,1-difluoroethyl)pyrimidine (135B, 890 mg, 84%) as a light yellow oil. LC/MS (ESI) m z: 213.1 [M+H]⁺.

To a mixture of 2,4-dichloro-5-(1,1-difluoroethyl)pyrimidine (135B, 680 mg, 3.21 mmol) and cis-cyclopentane-1,2-diol (360 mg, 3.53 mmol) in N,N-dimethylformamide (10 mL) at −30° C. under N₂ atmosphere with stirring, NaHMDS (1 M in THF, 3.85 mL, 3.85 mmol) was added into the mixture with temperature maintained between at −30° C. to −20° C. After addition, the mixture was stirred at −30° C. for additional 20 mins. The reaction was quenched with NH₄Cl saturated solution (3 mL) and H₂O (5 mL) and extracted with EtOAc (15 mL×3). The organic layers were combined, dried over anhydrous Na₂SO₄, and then concentrated under reduced pressure. The residue was separated using silica gel column chromatography to give cis-2-((2-chloro-5-(1,1-difluoroethyl)pyrimidin-4-yl)oxy)cyclopentan-1-ol (135C, 320 mg, 36%) as a white solids. LC/MS (ESI) m z: 279 [M+H]⁻.

A mixture of cis-2-((2-chloro-5-(1,1-difluoroethyl)pyrimidin-4-yl)oxy)cyclopentan-1-ol (135C, 278 mg, 1.00 mmol), 4-amino-N-isopropylbenzenesulfonamide (227 mg, 1.20 mmol), Cs₂CO₃ (975 mg, 3.00 mmol), Pd(OAc)₂ (67 mg, 0.30 mmol) and Xantphos (174 mg, 0.30 mmol) in dioxane (10 mL) was purged with N₂. The reaction was stirred at 100° C. for 1 hour under microwave irradiation. After completion, the reaction mixture was concentrated under reduced pressure and the residue was separated using silica gel column chromatography to give cis-4-((5-cyclopropyl-4-((2-hydroxycyclopentyl)oxy)pyrimidin-2-yl)amino)-N-isopropylbenzenesulfonamide (135) in a racemic form, which was further separated by Chiral SFC to give:

Enantiomer 1 (135a, 98.8% ee); Retention time: 5.25 min. LC-MS (ESI): m/z 412.2 [M+H]⁺; ¹H NMR (400 MHz, DMSO-d₆) δ 10.24 (s, 1H), 8.44 (s, 1H), 7.96 (d, J=8.8 Hz, 2H), 7.71 (d, J=8.8 Hz, 2H), 7.23 (s, 1H), 5.58-5.34 (m, 2H), 5.01 (d, J_{HF}=19.2 Hz, 1H), 4.86 (d, J=4.5 Hz, 1H), 4.32-4.16 (m, 1H), 2.18-2.03 (m, 1H), 1.97-1.73 (m, 3H), 1.68-1.56 (m, 2H).

Enantiomer 2 (135b, 99.6% ee); Retention time: 8.76 min. LC-MS (ESI): m/z 412.2 [M+H]⁺.

Analytical method: Column: ChiralPak IA, 250×4.6 mm I.D., 5 μm; Mobile phase: A for CO₂ and B for MeOH (0.05% DEA); Gradient: 8 min @B 40%; Flow rate: 2.0 mL/min; Back pressure: 100 bar; Column temperature: 35° C.

SFC Method: Instrument: Waters Thar 80 preparative SFC; Column: ChiralPak IA, 250×21.2 mm I.D., 5 μm; Mobile phase: A for CO₂ and B for MeOH+0.1% NH₃·H₂O; Gradient: B 50%; Flow rate: 40 mL/min; Back pressure: 100 bar; Column temperature: 35° C.; Wavelength: 220 nm; Cycle-time: 5 min; Eluted time: 1.2 hr.

Compounds of the present disclosure can be synthesized by those skilled in the art in view of the present disclosure. Representative further compounds synthesized by following similar procedures/methods described herein in the Examples section. Particularly, Example Nos. 9-64 were prepared by following similar procedures as shown above for Example Nos. 1-8 (Illustration 1-8); Example Nos. 67-91 were prepared by following similar procedures as shown above for Example Nos. 65 and 66 (Illustration 9, 10); Example Nos. 93-98 were prepared by following similar procedures as shown above for Example No. 92 (Illustration 11); Example Nos. 105-129 were prepared by following similar procedures as shown above for Example Nos. 99-104 (Illustration 12-17); and Example Nos. 136-155 were prepared by following similar procedures as shown above for Example Nos. 130-135 (Illustration 18-23). The structures and representative analytical data are shown in Table A below.

TABLE A
Characterization of exemplary compounds of the present disclosure

| Example | | LC-MS; ¹H NMR (ppm); |
|---|---|---|
| No. | Structure | Retention time |
| 9 | 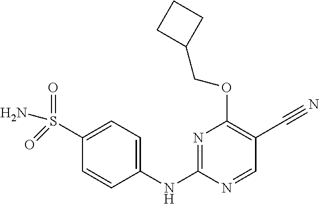 [SMILES: N#Cc1cnc(Nc2ccc(S(N)(=O)=O)cc2)nc1OCC1CCC1] | LC-MS (ESI): m/z 360.1 [M + H]⁺; ¹H NMR (400 MHz, DMSO-d₆) δ 10.65 (br s, 1H), 8.75 (s, 1H), 7.90 (d, J = 8.8 Hz, 2H), 7.80 (d, J = 8.8 Hz, 2H), 7.26 (br s, 2H), 4.48 (d, J = 6.6 Hz, 2H), 2.81-2.78 (m, 1H), 2.12-2.05 (m, 2H), 1.94-1.83 (m, 4H). |
| 10 | 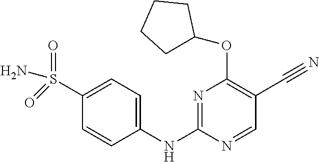 [SMILES: N#Cc1cnc(Nc2ccc(S(N)(=O)=O)cc2)nc1OC1CCCC1] | LC-MS (ESI): m/z 360.1 [M + H]⁺; ¹NMR (400 MHz, DMSO-d₆) δ 10.64 (br s, 1H), 8.73 (s, 1H), 7.91 (d, J = 8.8 Hz, 2H), 7.81 (d, J = 8.8 Hz, 2H), 7.25 (s, 2H), 5.57-5.54 (m, 1H), 2.08- 2.01 (m, 2H), 1.84-1.64 (m, 6H). |
| 11 | 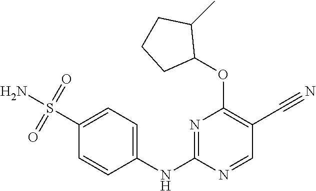 [SMILES: CC1CCCC1Oc1nc(Nc2ccc(S(N)(=O)=O)cc2)ncc1C#N] Mixture of isomers | LC-MS (ESI): m/z 374.1 [M + H]⁺: ¹H NMR (400 MHz, DMSO-d₆) δ 10.62 (br s, 1H), 8.73 (s, 1H), 7.89 (d, J = 8.8 Hz, 2H), 7.79 (d, J = 8.8 Hz, 2H), 7.25 (s, 2H), 5.13-5.09 (m, 1H), 2.19- 2.16 (m, 2H), 1.99-1.91 (m, 1H), 1.76-1.73 (m, 3H), 1.32-1.22 (m, 1H), 1.02 (d, J = 7.2 Hz, 3H). |
| 12 | 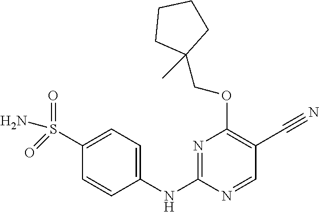 [SMILES: CC1(COc2nc(Nc3ccc(S(N)(=O)=O)cc3)ncc2C#N)CCCC1] | LC-MS (ESI): m/z 388.1 [M + H]⁺; ¹H NMR (400 MHz, DMSO-d₆) δ 10.65 (br s, 1H), 8.75 (s, 1H), 7.89 (d, J = 8.8 Hz, 2H), 7.78 (d, J = 8.8 Hz, 2H), 7.26 (s, 2H), 4.27 (s, 2H), 1.69-1.58 (m, 6H), 1.49-1.32 (m, 2H), 1.10 (s, 3H). |
| 13 | 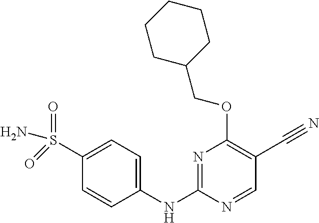 [SMILES: N#Cc1cnc(Nc2ccc(S(N)(=O)=O)cc2)nc1OCC1CCCCC1] | LS-MCS (ESI): m/z 388.1 [M + H]⁺; ¹H NMR (400 MHz, DMSO-d₆) δ 10.67 (br s, 1H), 8.74 (s, 1H), 7.89 (d, J = 8.8 Hz, 2H), 7.78 (d, J = 8.8 Hz, 2H), 7.27 (s, 2H), 4.30 (d, J = 6.4 Hz, 2H), 1.87-1.58 (m, 6H), 1.28-1.06 (m, 5H). |
| 14 | 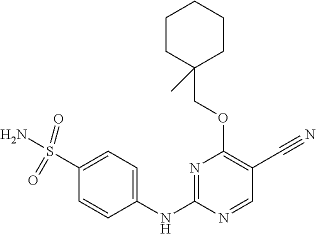 [SMILES: CC1(COc2nc(Nc3ccc(S(N)(=O)=O)cc3)ncc2C#N)CCCCC1] | LC-MS (ESI): m/z 402.2 [M + H]⁺; ¹H NMR (400 MHz, DMSO-d₆) δ 10.66 (br s, 1H), 8.75 (s, 1H), 7.89 (d, J = 8.8 Hz, 2H), 7.78 (d, J = 8.8 Hz, 2H), 7.26 (s, 2H), 4.25 (s, 2H), 1.67-1.13 (m, 10H), 1.02 (s, 3H). |
| 15 | 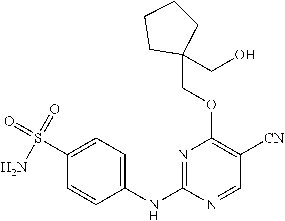 [SMILES: N#Cc1cnc(Nc2ccc(S(N)(=O)=O)cc2)nc1OCC1(CO)CCCC1] | LC-MS (ESI): m/z 404.1 [M + H]⁺; ¹H NMR (400 MHz, DMSO-d₆) δ 10.65 (br s, 1H), 8.75 (s, 1H), 7.91 (d, J = 8.8 Hz, 2H), 7.79 (d, J = 8.8 Hz, 2H), 7.25 (s, 2H), 4.79 (t, J = 5.2 Hz, 1H), 4.34 (s, 2H), 3.33 (d, J = 5.2 Hz, 2H), 1.61-1.48 (m, 8H). |
| 16 | 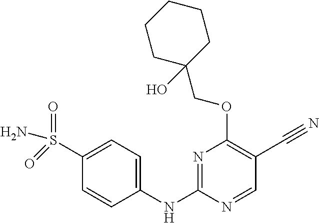 [SMILES: N#Cc1cnc(Nc2ccc(S(N)(=O)=O)cc2)nc1OCC1(O)CCCCC1] | LC-MS (ESI): m/z 404.1 [M + H]⁺; ¹H NMR (400 MHz, CD₃OD) δ 8.58 (s, 1H), 8.03-7.68 (m, 4H), 4.37 (s, 2H), 1.75-1.29 (m, 10H). |
| 17 | 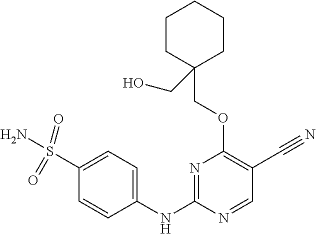 [SMILES: N#Cc1cnc(Nc2ccc(S(N)(=O)=O)cc2)nc1OCC1(CO)CCCCC1] | LC-MS (ESI): m/z 418.2 [M + H]⁺; ¹H NMR (400 MHz, DMSO-d₆) δ 10.65 (br s, 1H), 8.75 (s, 1H), 7.91 (d, J = 8.8 Hz, 2H), 7.78 (d, J = 8.8 Hz, 2H), 7.26 (s, 2H), 4.63 (t, J = 5.4 Hz, 1H), 4.34 (s, 2H), 3.39 (d, J = 5.4 Hz, 2H), 1.52-1.35 (m, 10H). |
| 18a | 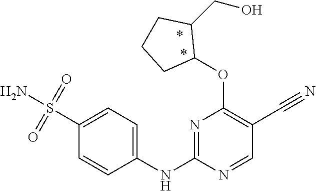 [SMILES: N#Cc1cnc(Nc2ccc(S(N)(=O)=O)cc2)nc1OC1CCCC1CO] Enantiomer 1 (earlier eluting enantiomer), from 2- (hydroxymethyl)cyclopentan-1-ol | LC-MS (EIS): m/z 390.1 [M + H]⁺; ¹H NMR (400 MHz, DMSO-d₆) δ 10.63 (br s, 1H), 8.72 (s, 1H), 7.90 (d, J = 8.8 Hz, 2H), 7.79 (d, J = 8.8 Hz, 2H), 7.24 (s, 2H), 5.60 (s, 1H), 4.51 (t, J = 5.2 Hz, 1H), 3.63-3.53 (m, 1H), 3.53-3.43 (m, 1H), 2.28-2.17 (m, 1H), 2.13-2.02 (m, 1H), 1.95-1.74 (m, 3H), 1.70-1.60 (m, 1H), 1.53- 1.43 (m, 1H). ee: 99.3% Retention time: 3.45 min; Column: ChiralPak AD, 250 × 4.6 mm I.D., 5 μm, Mobile phase: A for CO₂ and B for MeOH (0.05% |
| | | DEA), Gradient: 8 min @ B 40%, |
| | | Flow rate: 2.0 mL/min, Back |
| | | pressure: 100 bar, Column |
| | | temperature: 35° C. |
| 18b | 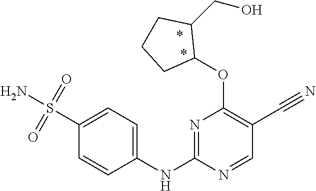 [SMILES: N#Cc1cnc(Nc2ccc(S(N)(=O)=O)cc2)nc1OC1CCCC1CO] Enantiomer 2 (later eluting enantiomer), from 2- (hydroxymethyl)cyclopentan-1-ol | LC-MS (ESI): m/z 390.1 [M + H]⁺; ¹H NMR (400 MHz, DMSO-d₆) δ 10.63 (br s, 1H), 8.72 (s, 1H), 7.90 (d, J = 8.8 Hz, 2H), 7.79 (d, J = 8.8 Hz, 2H), 7.24 (s, 2H), 5.59 (s, 1H), 4.51 (t, J = 5.2 Hz, 1H), 3.63-3.54 (m, 1H), 3.52-3.44 (m, 1H), 2.26-2.18 (m, 1H), 2.14-2.03 (m, 1H), 1.93-1.73 (m, 3H), 1.69-1.62 (m, 1H), 1.54- 1.43 (m, 1H). ee: 89.1% Retention time: 4.00 min; Column: ChiralPak AD, 250 × 4.6 mm I.D., 5 μm, Mobile phase: A for CO₂ and B for MeOH (0.05% |
| | | DEA), Gradient: 8 min @ B 40%, |
| | | Flow rate: 2.0 mL/min, Back |
| | | pressure: 100 bar, Column |
| | | temperature: 35° C. |
| 19a | 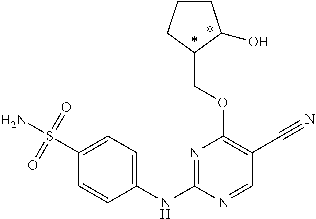 [SMILES: N#Cc1cnc(Nc2ccc(S(N)(=O)=O)cc2)nc1OCC1CCCC1O] Enantiomer 1 (earlier eluting enantiomer), from 2- (hydroxymethyl)cyclopentan-1-ol | LC-MS (ESI): m/z 390.1 [M + H]⁺; ¹H NMR (400 MHz, DMSO-d₆) δ 10.65 (br s, 1H), 8.74 (s, 1H), 7.91 (d, J = 9.0 Hz, 2H), 7.78 (d, J = 9.0 Hz, 2H), 7.25 (s, 2H), 4.70-4.54 (m, 2H), 4.44 (m, 1H), 4.17 (s, 1H), 2.20 (s, 1H), 1.85-1.72 (m, 3H), 1.65-1.48 (m, 3H). ee: 96.7% Retention time: 7.12 min; Column: ChiralPak AD, 250 × 4.6 mm I.D., 5 μm, Mobile phase: A for CO2 and B for MeOH (0.05% DEA), Gradient: 8 min @ B 40%, Flow rate: 2.0 mL/min, Back pressure: 100 bar, Column temperature: 35° C. |
| 19b | 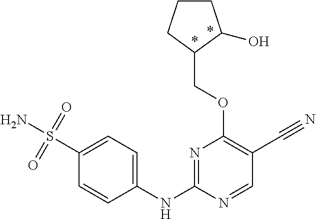 [SMILES: N#Cc1cnc(Nc2ccc(S(N)(=O)=O)cc2)nc1OCC1CCCC1O] Enantiomer 2 (later eluting enantiomer), from 2- (hydroxymethyl)cyclopentan-1-ol | LC-MS (ESI): m/z 390.1 [M + H]⁺; ¹H NMR (400 MHz, DMSO-d₆) δ 10.65 (br s, 1H), 8.74 (s, 1H), 7.91 (d, J = 8.8 Hz, 2H), 7.78 (d, J = 8.8 Hz, 2H), 7.25 (s, 2H), 4.66-4.55 (m, 2H), 4.49- 4.39 (m, 1H), 4.17 (s, 1H), 2.28- 2.16 (m, 1H), 1.84-1.70 (m, 3H), 1.67-1.44 (m, 3H). ee: 89.1% Retention time: 8.27 min; Column: ChiralPak AD, 250 × 4.6 mm I.D., 5 μm, Mobile phase: A for CO₂ and B for MeOH (0.05% DEA), Gradient: 8 min @ B 40%, Flow rate: 2.0 mL/min, Back pressure: 100 bar, Column temperature: 35° C, |
| 20a | 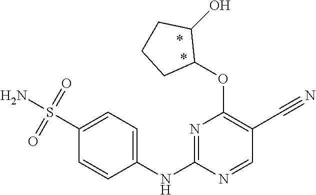 [SMILES: N#Cc1cnc(Nc2ccc(S(N)(=O)=O)cc2)nc1OC1CCCC1O] Enantiomer 1 (earlier eluting enantiomer), from cis-cyclopentane 1,2-diol | LC-MS (ESI): m/z 376.1 [M + H]⁺; ¹H NMR (400 MHz, DMSO-d₆) δ 10.60 (br s, 1H), 8.73 (s, 1H), 7.89 (d, J = 8.8 Hz, 2H), 7.79 (d, J = 8.8 Hz, 2H), 7.26 (s, 2H), 5.34-5.30 (m, 1H), 4.83 (d, J = 4.8 Hz, 1H), 4.26-4.24 (m, 1H), 2.04-1.98 (m, 1H), 1.89-1.78 (m, 3H), 1.68-1.51 (m, 2H). Retention time: 3.85 min; Column: ChiralCel OD, 250 × 4.6 mm I.D., 5 μm; Mobile phase: A for CO₂ and B for methanol (0.05% DEA); Gradient: 10 min @ 40%; Flow rate: 2.0 mL/min; Column temperature: 35° C. |
| 20b | 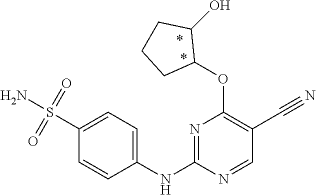 [SMILES: N#Cc1cnc(Nc2ccc(S(N)(=O)=O)cc2)nc1OC1CCCC1O] Enantiomer 2 (later eluting enantiomer), from cis-cyclopentane 1,2-diol | LC-MS (ESI): m/z 376.1 [M + H]⁺; ¹H NMR (400 MHz, DMSO-d₆) δ 10.61 (s, 1H), 8.73 (s, 1H), 7.89 (d, J = 8.8 Hz, 2H), 7.79 (d, J = 8.8 Hz, 2H), 7.26 (s, 2H), 5.34-5.29 (m, 1H), 4.84 (d, J = 4.8 Hz, 1H), 4.26-4.24 (m, 1H), 2.07-1.99 (m, 1H), 1.86-1.75 (m, 3H), 1.68-1.52 (m, 2H). Retention time: 4.76 min; Column: ChiralCel OD, 250 × 4.6 mm I.D., 5 μm; Mobile phase: A for CO₂ and B for methanol (0.05% DEA); Gradient: 10 min @ 40%; Flow rate: 2.0 mL/min; Column temperature: 35° C. |
| 21a | 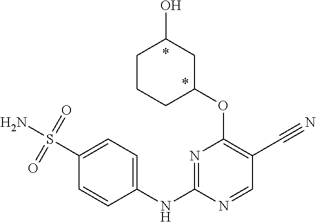 [SMILES: N#Cc1cnc(Nc2ccc(S(N)(=O)=O)cc2)nc1OC1CCCC(O)C1] Enantiomer 1 (earlier eluting enantiomer), from trans- cyclohexane-1,3-diol | LC-MS (ESI): m/z 390.0 [M + H]⁺; ¹H NMR (400 MHz, DMSO-d₆) δ 10.67 (br s, 1H), 8.74 (s, 1H), 7.87 (d, J = 8.8 Hz, 2H), 7.78 (d, J = 8.8 Hz, 2H), 7.26 (s, 2H), 5.09 (br s, 1H), 4.83 (d, J = 4.6 Hz, 1H), 3.55 (s, 1H), 2.38- 2.32 (m, 1H), 2.13-2.07 (m, 1H), 1.90-1.73 (m, 2H), 1.34-1.15 (m, 4H). ee: 100% Retention time: 5.15 min; Column: ChiralPak IC, 250 × 4.6 mm I.D., 5 μm; Mobile phase: A for CO₂ and B for EtOH (0.05% DEA), Gradient: 8 min @ B 50%, Flow rate: 1.8 mL/min, Back pressure: 100 bar, Column |
| | | temperature: 35° C. |
| 21b | 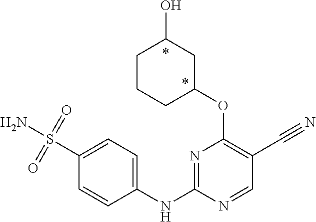 [SMILES: N#Cc1cnc(Nc2ccc(S(N)(=O)=O)cc2)nc1OC1CCCC(O)C1] Enantiomer 2 (later eluting enantiomer), from trans- cyclohexane-1,3-diol | LC-MS (ESI): m/z 390.0 [M + H]⁺; ¹H NMR (400 MHz, DMSO-d₆) δ 10.66 (br s, 1H), 8.74 (s, 1H), 7.87 (d, J = 8.8 Hz, 2H), 7.78 (d, J = 8.8 Hz, 2H), 7.26 (s, 2H), 5.09 (br s, 1H), 4.83 (d, J = 4.8 Hz, 1H), 3.56 (s, 1H), 2.40- 2.32 (m, 1H), 2.14-2.06 (m, 1H), 1.91-1.73 (m, 2H), 1.36-1.15 (m, 4H). ee: 95.1% Retention time: 6.10 min; Column: ChiralPak IC, 250 × 4.6 mm I.D., 5 μm, Mobile phase: A for CO₂ and B for EtOH (0.05% DEA), Gradient: 8 min @ B 50%, Flow rate: 1.8 mL/min, Back pressure: 100 bar, Column |
| | | temperature: 35° C. |
| 22a | 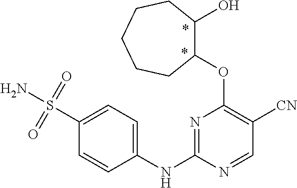 [SMILES: N#Cc1cnc(Nc2ccc(S(N)(=O)=O)cc2)nc1OC1CCCCCC1O] Enantiomer 1 (earlier eluting enantiomer), made from cis- cycloheptane-1,2-diol | LC-MS (ESI): m/z 404.1 [M + H]⁺; ¹H NMR (400 MHz, DMSO-d₆) δ 10.61 (br s, 1H), 8.74 (s, 1H), 7.87 (d, J = 8.8 Hz, 2H), 7.77 (d, J = 8.8 Hz, 2H), 7.26 (s, 2H), 5.40 (d, J = 8.0 Hz, 1H), 4.84 (d, J = 4.8 Hz, 1H), 4.00 (s, 1H), 2.14-2.03 (m, 1H), 1.83-1.49 (m, 9H). ee: 54.8% Retention time: 3.94 min. Column: ChiralPak AD, 250 × 4.6 mm I.D., 5 μm, Mobile phase: A for CO₂ and B for methanol (0.05% DEA), Gradient: 10 min @ 40%. Flow rate: 2.0 mL/min, |
| | | Back pressure: 100 bar, Column |
| | | temperature: 35° C. |
| 22b | 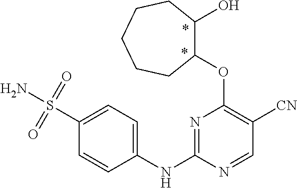 [SMILES: N#Cc1cnc(Nc2ccc(S(N)(=O)=O)cc2)nc1OC1CCCCCC1O] Enantiomer 2 (later eluting enantiomer), made from cis- cycloheptane-1,2-diol | LC-MS (ESI): m/z 404.0 [M + H]⁺; ¹H NMR (400 MHz, DMSO-d₆) δ 10.61 (br s, 1H), 8.74 (s, 1H), 7.87 (d, J = 8.8 Hz, 2H), 7.77 (d, J = 8.9 Hz, 2H), 7.26 (s, 2H), 5.40 (d, J = 8.0 Hz, 1H), 4.84 (d, J = 4.7 Hz, 1H), 3.99 (s, 1H), 2.12-2.04 (m, 1H), 1.83-1.49 (m, 9H). ee: 25.3% Retention time: 4.94 min; Column: ChiralPak AD, 250 × 4.6 mm I.D., 5 μm, Mobile phase: A for CO₂ and B for MeOH (0.05% DEA), Gradient: 8 min @ B 40%, |
| | | Flow rate: 2.0 mL/min, Back |
| | | pressure: 100 bar, Column |
| | | temperature: 35° C. |
| 23a | 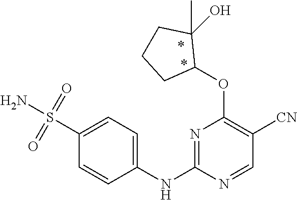 [SMILES: CC1(O)CCCC1Oc1nc(Nc2ccc(S(N)(=O)=O)cc2)ncc1C#N] Enantiomer 1 (earlier eluting enantiomer), made from cis-1- methylcyclopentane-1,2-diol | LC-MS (EIS): m/z 390.1 [M + H]⁺; ¹H NMR (400 MHz, DMSO-d₆) δ 10.58 (br s, 1H), 8.73 (s, 1H), 7.90 (d, J = 8.8 Hz, 2H), 7.78 (d, J = 8.8 Hz, 2H), 7.26 (s, 2H), 5.25-5.18 (m, 1H), 4.63 (s, 1H), 2.20-2.12 (m, 1H), 1.86- 1.75 (m, 3H), 1.65-1.52 (m, 2H), 1.25 (s, 3H). ee: 98.0% Retention time: 2.66 min. Column: ChiralPak AD, 250 × 4.6 mm I.D., 5 μm, Mobile phase: A for CO₂ and B for methanol (0.05% DEA), Gradient: 10 min @ 40%. Flow rate: 2.0 mL/min, Back pressure: 100 bar, Column |
| | | temperature: 35° C. |
| 23b | 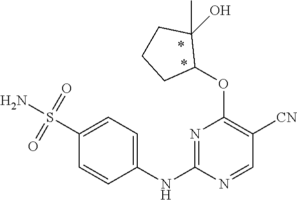 [SMILES: CC1(O)CCCC1Oc1nc(Nc2ccc(S(N)(=O)=O)cc2)ncc1C#N] Enantiomer 2 (later eluting enantiomer), made from cis-1- methylcyclopentane-1,2-diol | LC-MS (ESI): m/z 390.0 [M + H]⁺; ¹H NMR (400 MHz, DMSO-d₆) δ 10.57 (br s, 1H), 8.73 (s, 1H), 7.90 (d, J = 8.8 Hz, 2H), 7.78 (d, J = 8.8 Hz, 2H), 7.26 (s, 2H), 5.24-5.19 (m, 1H), 4.63 (s, 1H), 2.20-2.11 (m, 1H), 1.85- 1.78 (m, 3H), 1.65-1.56 (m, 2H), 1.25 (s, 3H). ee: 95.2% Retention time: 3.47 min; Column: ChiralPak AD, 250 × 4.6 mm I.D., 5 μm, Mobile phase: A for CO₂ and B for MeOH (0.05% DEA), Gradient: 8 min @ B 40%, Flow rate: 2.0 mL/min, Back pressure: 100 bar, Column |
| | | temperature: 35° C. |
| 24a | 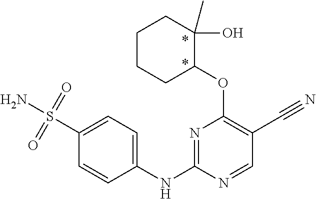 [SMILES: CC1(O)CCCCC1Oc1nc(Nc2ccc(S(N)(=O)=O)cc2)ncc1C#N] Enantiomer 1 (earlier eluting enantiomer), made from cis-1- methylcyclohexane-1,2-diol | LC-MS (ESI): m/z 404.0 [M + H]⁺; ¹H NMR (400 MHz, DMSO-d₆) δ 10.61 (br s, 1H), 8.74 (s, 1H), 7.88 (d, J = 8.8 Hz, 2H), 7.77 (d, J = 8.8 Hz, 2H), 7.26 (s, 2H), 5.04 (s, 1H), 4.47 (s, 1H), 1.86-1.81 (m, 2H), 1.77-1.62 (m, 3H), 1.44-1.41 (m, 3H), 1.17 (s, 3H). ee: 99.5% Retention time: 2.56 min; Column: ChiralPAK AD, 250 × 21.2 mm I.D., 5 μm; Mobile phase: A for CO₂ and B for methanol (0.05% DEA); Gradient: 10 min @ 40%; Flow rate: 40 mL/min; Column |
| | | temperatuere: 35° C. |
| 24b | 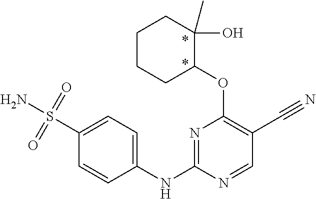 [SMILES: CC1(O)CCCCC1Oc1nc(Nc2ccc(S(N)(=O)=O)cc2)ncc1C#N] | LC-MS (ESI): m/z 404.0 [M + H]⁺. ee: 97.0% Retention time: 3.37 min; Column: ChiralPAK AD, 250 × 21.2 mm I.D., 5 μm; Mobile phase: A for CO₂ and B for methanol (0.05% DEA); Gradient: 10 min @ 40%; Flow rate: 40 mL/min; Column temperature: 35° C. |
| | Enantiomer 2 (later eluting | |
| | enantiomer), made from cis-1- | |
| | methylcyclohexane-1,2-diol | |
| 25 | 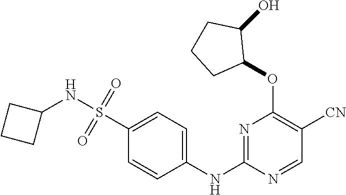 Racemic mixture | ¹H NMR (500 MHz, DMSO-d₆) δ 10.63 (s, 1H), 8.74 (s, 1H), 7.88 (d, J = 8.5 Hz, 2H), 7.83 (d, J = 8.5 Hz, 1H), 7.73 (d, J = 8.5 Hz, 2H), 5.31 (q, J = 5.5 Hz, 1H), 4.81 (s, 1H), 4.24 (d, J = 6.5 Hz, 1H), 3.69-3.49 (m, 1H), 2.06-1.97 (m, 1H), 1.92-1.79 (m, 5H), 1.75-1.63 (m, 3H), 1.57-1.39 (m, 3H). |
| 26 | 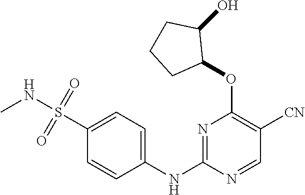 Racemic mixture | LC-MS (ESI): m/z 390.1 [M + H]⁺; ¹H NMR (500 MHz, DMSO-d₆) δ 10.65 (s, 1H), 8.75 (s, 1H), 7.92 (d, J = 8.5 Hz, 2H), 7.74 (d, J = 8.5 Hz, 2H), 7.32 (d, J = 5.0 Hz, 1H), 5.33 (q, J = 5.5 Hz, 1H), 4.36 (s, 1H), 4.32-4.18 (m, 1H), 2.40 (d, J = 5.0 Hz, 3H), 2.09-1.98 (m, 1H), 1.91-1.76 (m, 3H), 1.69-1.59 (m, 1H), 1.58-1.48 (m, 1H). |
| 27 | 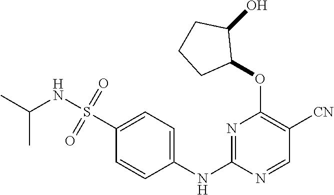 Racemic mixture | LC-MS (ESI): m/z 418.1 [M + H]⁺; ¹H NMR (500 MHz, DMSO-d₆) δ 10.63 (s, 1H), 8.75 (d, J = 2.5 Hz, 1H), 7.90 (d, J = 8.5 Hz, 2H), 7.77 (d, J = 9.0 Hz, 2H), 7.46 (d, J = 7.5 Hz, 1H), 5.50-5.23 (m, 1H), 4.81 (s, 1H), 4.25 (q, J = 5.0 Hz, 1H), 3.29-3.09 (m, 1H), 2.11-1.98 (m, 1H), 1.93- 1.76 (m, 3H), 1.71-1.43 (m, 1H), 1.60-1.44 (m, 1H), 0.95 (d, J = 6.5 Hz, 6H). |
| 28 | 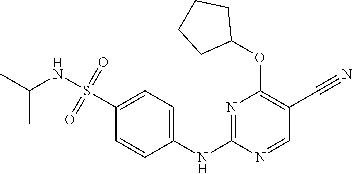 [SMILES: CC(C)NS(=O)(=O)c1ccc(Nc2ncc(C#N)c(OC3CCCC3)n2)cc1] | LC-MS (ESI): m/z 402.2 [M + H]⁺; ¹H NMR (400 MHz, DMSO-d₆) δ 10.67 (s, 1H), 8.73 (s, 1H), 7.92 (d, J = 8.8 Hz, 2H), 7.77 (d, J = 8.8 Hz, 2H), 7.44 (d, J = 7.2 Hz, 1H), 5.65-5.47 (m, 1H), 3.27-3.17 (m, 1H), 2.04-2.19 (m, 2H), 1.76-1.62 (m, 6H), 0.94 (d, J = 6.5 Hz, 6H). |
| 29a | 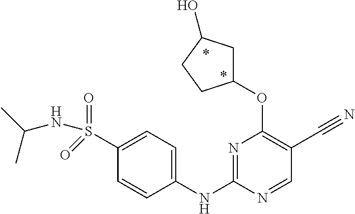 [SMILES: CC(C)NS(=O)(=O)c1ccc(Nc2ncc(C#N)c(OC3CCC(O)C3)n2)cc1] Enantiomer 1 (earlier eluting enantiomer), made from trans- cyclopentane-1,3-diol | LC-MS (ESI): m/z 418.2 [M + H]⁺; ¹H NMR (400 MHz, DMSO-d₆) δ 10.68 (s, 1H), 8.73 (s, 1H), 7.92 (d, J = 8.8 Hz, 2H), 7.77 (d, J = 8.8 Hz, 2H), 7.45 (d, J = 7.2 Hz, 1H), 5.78-5.41 (m, 1H), 4.72 (d, J = 3.6 Hz, 1H), 4.30 (d, J = 3.6 Hz, 1H), 3.24-3.19 (m, 1H), 2.33-2.18 (m, 1H), 2.11-2.02 (m, 1H), 2.01-1.87 (m, 2H), 1.82- 1.72 (m, 1H), 1.62-1.52 (m, 1H), 0.94 (d, J = 6.4 Hz, 6H). ee: 100% Retention time: 2.95 min; Column: ChiralPak AD, 250 × 4.6 mm I.D., 5 μm, Mobile phase: A for CO₂ and B for methanol (0.05% DEA), Gradient: 10 min |
| | | @ 40%, Flow rate: 2.0 mL/min, |
| | | Back pressure: 100 bar, Column |
| | | temperature: 35° C. |
| 29b | 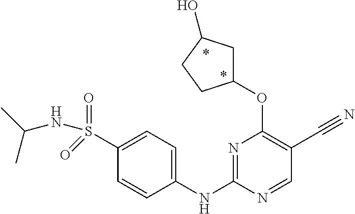 [SMILES: CC(C)NS(=O)(=O)c1ccc(Nc2ncc(C#N)c(OC3CCC(O)C3)n2)cc1] Enantiomer 2 (later eluting enantiomer), made from trans- cyclopentane-1,3-diol | LC-MS (ESI): m/z 418.2 [M + H]⁺. ee: 97.4% Retention time: 3.88 min; Column: ChiralPak AD, 250 × 4.6 mm I.D., 5 μm, Mobile phase: A for CO₂ and B for methanol (0.05% DEA), Gradient: 10 min @ 40%, Flow rate: 2.0 mL/min, Back pressure: 100 bar, Column temperature: 35° C. |
| 30a | 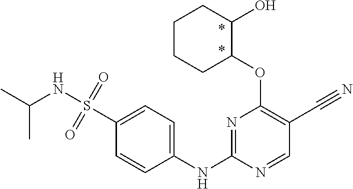 [SMILES: CC(C)NS(=O)(=O)c1ccc(Nc2ncc(C#N)c(OC3CCCCC3O)n2)cc1] Enantiomer 1 (earlier eluting enantiomer), made from cis- | LC-MS (ESI): m/z 432.2 [M + H]⁺; ¹H NMR (400 MHz, DMSO-d₆) δ 10.63 (s, 1H), 8.74 (s, 1H), 7.88 (d, J = 8.8 Hz, 2H), 7.75 (d, J = 8.8 Hz, 2H), 7.46 (d, J = 7.0 Hz, 1H), 5.32 (s, 1H), 4.83 (d, J = 4.8 Hz, 1H), 3.91 (s, 1H), 3.24-3.13 (m, 1H), 2.01-1.92 (m, 1H), 1.72-1.52 (m, 5H), 1.43-1.31 (m, 2H), 0.94 (d, J = 6.4 Hz, 6H). ee: 97.2% Retention time: 4.44 min; Column: ChiralPak IA, 250 × 4.6 mm I.D., 5 m, Mobile phase: A |
| | cyclohexane-1,2-diol | for CO₂ and B for MeOH (0.05% |
| | | DEA), Gradient: 8 min @ B 40%, |
| | | Flow rate: 1.8 mL/min, Back |
| | | pressure: 100 bar, Column |
| | | temperature: 35° C. |
| 30b | 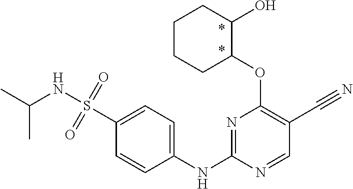 [SMILES: CC(C)NS(=O)(=O)c1ccc(Nc2ncc(C#N)c(OC3CCCCC3O)n2)cc1] Enantiomer 2 (later eluting enantiomer), made from cis- cyclohexane-1,2-diol | LC-MS (ESI): m/z 432.2 [M + H]⁺. ee: 95.1% Retention time: 5.99 min; Column: ChiralPak IA, 250 × 4.6 mm I.D., 5 μm, Mobile phase: A for CO₂ and B for MeOH (0.05% DEA), Gradient: 8 min @ B 40%, Flow rate: 1.8 mL/min, Back pressure: 100 bar, Column temperature: 35° C. |
| 31a | 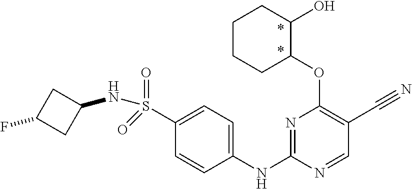 Enantiomer 1 (earlier eluting enantiomer), made from cis- cyclohexane-1,2-diol | LC-MS (ESI): m/z 462.2 [M + H]⁺; ¹H NMR (400 MHz, DMSO-d₆) δ 10.65 (s, 1H), 8.75 (s, 1H), 7.95-7.84 (m, 3H), 7.73 (d, J = 8.8 Hz, 2H), 5.33 (s, 1H), 5.19-4.93 (m, 1H), 4.83 (d, J = 4.,7 Hz, 1H), 3.95-3.76 (m, 2H), 2.34- 2.03 (m, 4H), 2.02-1.90 (m, 1H), 1.81-1.47 (m, 5H), 1.45-1.25 (m, 2H). ee: 97.9% Retention time: 3.50 min; Column: ChiralCel OD, 250 × 4.6 mm I.D., 5 μm, Mobile phase: A for CO₂ and B for MeOH (0.05% DEA), Gradient: 8 min @ B 40%, |
| | | Flow rate: 2.0 mL/min, Back |
| | | pressure: 100 bar, Column |
| | | temperature: 35° C. |
| 31b | 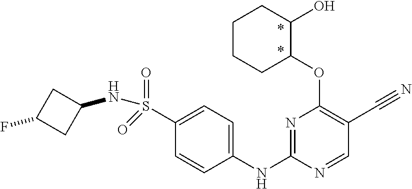 Enantiomer 2 (later eluting enantiomer), made from cis- cyclohexane-1,2-diol | LC-MS (ESI): m/z 462.2 [M + H]⁺. ee: 95.5% Retention time: 4.20 min; Column: ChiralCel OD, 250 × 4.6 mm I.D., 5 μm, Mobile phase: A for CO₂ and B for MeOH (0.05% DEA), Gradient: 8 min @ B 40%, Flow rate: 2.0 mL/min, Back pressure: 100 bar, Column temperature: 35° C. |
| 32a | 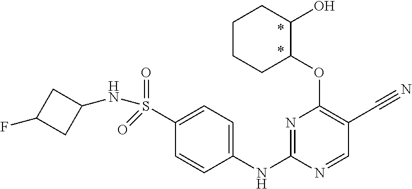 [SMILES: N#Cc1cnc(Nc2ccc(S(=O)(=O)NC3CC(F)C3)cc2)nc1OC1CCCCC1O] Enantiomer 1 (earlier eluting enantiomer), made from cis- cyclohexane-1,2-diol | LC-MS (ESI): m/z 480.1 [M + H]⁺; ¹H NMR (400 MHz, DMSO-d₆) δ 10.67 (br s, 1H), 8.75 (s, 1H), 8.05 (s, 1H), 7.91 (d, J = 8.8 Hz, 2H), 7.77 (d, J = 8.8 Hz, 2H), 5.34 (s, 1H), 4.84 (d, J = 4.6 Hz, 1H), 3.91 (s, 1H), 3.61- 3.49 (m, 1H), 2.73-2.66 (m, 2H), 2.42-2.32 (m, 2H), 2.02-1.90 (m, 1H), 1.77-1.51 (m, 5H), 1.44-1.31 (m, 2H). ee: 98.7% Retention time: 3.09 min. Column: ChiralPak AD, 250 × 4.6 mm I.D., 5 μm, Mobile phase: A for CO₂ and B for methanol |
| | | (0.05% DEA), Gradient: 10 min |
| | | @ 50%. Flow rate: 1.8 mL/min, |
| | | back pressure: 100 bar, Column |
| | | temperature: 35° C. |
| 32b | 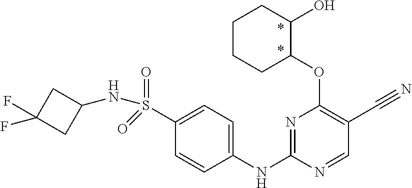 [SMILES: N#Cc1cnc(Nc2ccc(S(=O)(=O)NC3CC(F)(F)C3)cc2)nc1OC1CCCCC1O] Enantiomer 2 (later eluting enantiomer), made from cis- cyclohexane-1,2-diol | LC-MS (ESI): m/z 480.0 [M + H]⁺. ee: 97.3% Retention time: 4.71 min; Column: ChiralPak AD, 250 × 4.6 mm I.D., 5 μm, Mobile phase: A for CO₂ and B for methanol (0.05% DEA), Gradient: 10 min @ 50%, Flow rate: 1.8 mL/min, Back pressure: 100 bar, Column temperature: 35° C. |
| 33a | 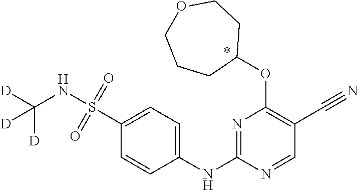 [SMILES: [2H]C([2H])([2H])NS(=O)(=O)c1ccc(Nc2ncc(C#N)c(OC3CCCOCC3)n2)cc1] Enantiomer 1 (earlier eluting enantiomer), made from oxepan-4- ol | LC-MS (ESI): m/z 407.2 [M + H]⁺; ¹H NMR (400 MHz, DMSO-d₆) δ 8.75 (s, 1H), 7.92 (d, J = 8.8 Hz, 2H), 7.74 (d, J = 8.8 Hz, 2H), 7.32 (s, 1H), 5.49-5.36 (m, 1H), 3.88-3.58 (m, 4H), 2.23- 2.14 (m, 1H), 2.10-2.07 (m, 1H), 2.05-1.94 (m, 2H), 1.87-1.85 (m, 1H), 1.71-1.69 (m, 1H). ee: 100% Retention time: 1.50 min; Column: Chiralpak IG-3, 100 × 4.6 mm I.D., 3 μm, Mobile Phase: 40% of ethanol (0.05% DEA) in CO₂, Flow rate: 2.5 mL/min, Column temperature: 35° C. |
| 33b | 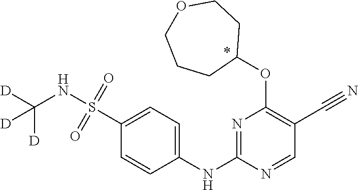 [SMILES: [2H]C([2H])([2H])NS(=O)(=O)c1ccc(Nc2ncc(C#N)c(OC3CCCOCC3)n2)cc1] Enantiomer 2 (later eluting enantiomer), made from oxepan-4- ol | LC-MS (EIS): m/z 407.2 [M + H]⁺. ee: 100% Retention time: 2.17 min; Column: Chiralpak IG-3, 100 × 4.6 mm I.D., 3 μm, Mobile phase: 40% of ethanol (0.05% DEA) in CO₂, Flow rate: 2.5 mL/min, Column temperature: 35° C. |
| 34a | 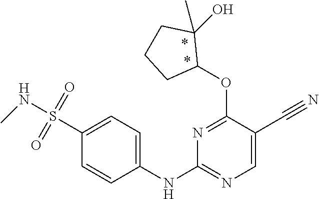 [SMILES: CNS(=O)(=O)c1ccc(Nc2ncc(C#N)c(OC3CCCC3(C)O)n2)cc1] Enantiomer 1 (earlier eluting enantiomer), made from cis-1- methylcyclopentane-1,2-diol | LC-MS (ESI): m/z 404.2 [M + H]⁺; ¹H NMR (400 MHz, DMSO-d₆) δ 10.61 (s, 1H), 8.74 (s, 1H), 7.93 (d, J = 8.0 Hz, 2H), 7.73 (d, J = 8.0 Hz, 2H), 7.34-7.30 (m, 1H), 5.21 (q, J = 4.0 Hz, 1H), 4.61 (s, 1H), 2.40 (d, J = 4.0 Hz, 3H), 2.20-2.10 (m, 1H), 1.86-1.76 (m, 3H), 1.69-1.53 (m, 2H), 1.23 (s, 3H). ee: 98.8% Retention time: 2.71 min; Column: ChiralPaAK AD, 250 × 21.2 mm I.D., 5 μm; Mobile phase: A for CO₂ and B for methanol (0.05% DEA); |
| | | Gradient: 10 min a 40%; Flow |
| | | rate: 40 mL/min; Column |
| | | temperature: 35° C. |
| 34b | 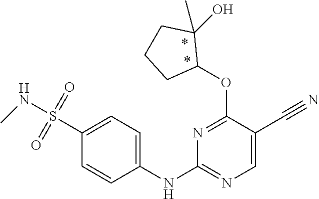 [SMILES: CNS(=O)(=O)c1ccc(Nc2ncc(C#N)c(OC3CCCC3(C)O)n2)cc1] Enantiomer 2 (later eluting enantiomer), made from cis-1- methylcyclopentane-1,2-diol | LC-MS (ESI): m/z 404.2 [M + H]⁺; ¹H NMR (400 MHz, DMSO-d₆) δ 10.62 (s, 1H), 8.74 (s, 1H), 7.94 (d, J = 8.0 Hz, 2H), 7.74 (d, J = 8.0 Hz, 2H), 7.33 (q, J = 4.0 Hz, 1H), 5.24-5.20 (m, 1H), 4.62 (s, 1H), 2.41 (d, J = 5.2 Hz, 3H), 2.22-2.10 (m, 1H), 1.88- 1.76 (m, 3H), 1.67-1.52 (m, 2H), 1.24 (s, 3H). ee: 91.3% Retention time: 3.54 min; Column: ChiralPAK AD, 250 × 21.2 mm I.D., 5 μm; Mobile phase: A for CO₂ and B for methanol (0.05% DEA); Gradient: 10 min a 40%; Flow |
| | | rate: 40 mL/min; Column |
| | | temperature: 35° C. |
| 35a | 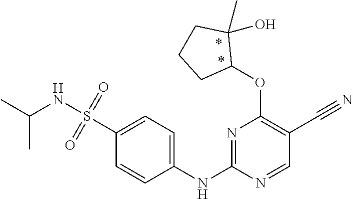 [SMILES: CC(C)NS(=O)(=O)c1ccc(Nc2ncc(C#N)c(OC3CCCC3(C)O)n2)cc1] Enantiomer 1 (earlier eluting enantiomer), made from cis-1- methylcyclopentane-1,2-diol | LC-MS (ESI): m/z 432.2 [M + H]⁺; ¹H NMR (400 MHz, DMSO-d₆) δ 10.60 (s, 1H), 8.73 (s, 1H), 7.91 (d, J = 8.8 Hz, 2H), 7.76 (d, J = 8.8 Hz, 2H), 7.46 (d, J = 7.2 Hz, 1H), 5.22-5.19 (m, 1H), 4.62 (s, 1H), 3.26-3.19 (m, 1H), 2.16-2.06 (m, 1H), 1.83-1.80 (m, 3H), 1.62-1.57 (m, 2H), 1.23 (s, 3H), 0.94 (d, J = 6.4 Hz, 6H). ee: 98.5% Retention time: 2.75 min; Column: ChiralPak AD, 250 × 4.6 mm I.D., 5 μm, Mobile phase: A for CO₂ and B for MeOH (0.05% DEA), Gradient: 8 min @ B 30%, |
| | | Flow rate: 2.0 mL/min, Back |
| | | pressure: 100 bar, Column |
| | | temperature: 35° C. |
| 35b | 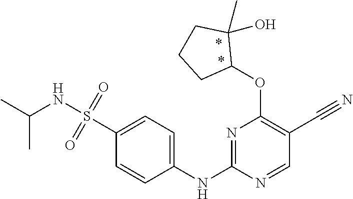 [SMILES: CC(C)NS(=O)(=O)c1ccc(Nc2ncc(C#N)c(OC3CCCC3(C)O)n2)cc1] Enantiomer 2 (later eluting enantiomer), made from cis-1- methylcyclopentane-1,2-diol | LC-MS (ESI): m/z 432.2 [M + H]⁺. ee: 93.4% Retention time: 3.70 min; Column: ChiralPak AD, 250 × 4.6 mm I.D., 5 μm, Mobile phase: A for CO₂ and B for MeOH (0.05% DEA), Gradient: 8 min @ B: 30%, Flow rate: 2.0 mL/min, Back pressure: 100 bar, Column temperature: 35° C. |
| 36a | 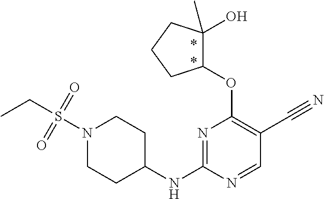 [SMILES: CCS(=O)(=O)N1CCC(Nc2ncc(C#N)c(OC3CCCC3(C)O)n2)CC1] Enantiomer 1 (earlier eluting enantiomer), made from cis-1- methylcyclopentane-1,2-diol | LC-MS (ESI): m/z 410.3 [M + H]⁺. ee: 100% Retention time: 2.37 min; Column: ChiralPak AD, 250 × 4.6 mm I.D., 5 μm, Mobile phase: A for CO₂ and B for methanol (0.05% DEA), Gradient: 10 min @ 40%, Flow rate: 2.0 mL/min, Back pressure: 100 bar, Column temperature: 35° C. |
| 36b | 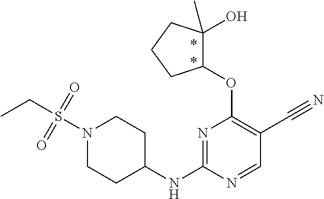 [SMILES: CCS(=O)(=O)N1CCC(Nc2ncc(C#N)c(OC3CCCC3(C)O)n2)CC1] Enantiomer 2 (later eluting enantiomer), made from cis-1- methylcyclopentane-1,2-diol | LC-MS (ESI): m/z 410.3 [M + H]⁺; ¹H NMR (400 MHz, DMSO-d₆)(tautomer ratio = 1:1) δ 8.51 & 8.44 (s, 1H), 8.26 & 8.06 (d, J = 7.7 Hz, 1H), 5.15-5.06 (m, 1H), 4.47 & 4.45 (s, 1H), 3.98- 3.84 (m, 1H), 3.60-3.56 (m, 2H), 3.08-3.02 (m, 2H), 2.99-2.79 (m, 2H), 2.17-2.00 (m, 1H), 1.92-1.74 (m, 5H), 1.66-1.42 (m, 4H), 1.22- 1.18 (m, 6H). ee: 89.1% Retention time: 3.13 min; Column: ChiralPak AD, 250 × 4.6 mm I.D., 5 μm, Mobile phase: A for CO₂ and B for methanol |
| | | (0.05% DEA), Gradient: 10 min |
| | | @ 40%, Flow rate: 2.0 mL/min, |
| | | Back pressure: 100 bar, Column |
| | | temperature: 35° C. |
| 37a | 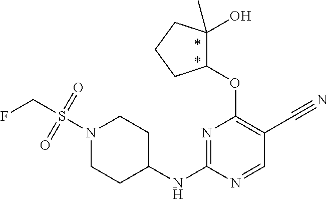 [SMILES: CC1(O)CCCC1Oc1nc(NC2CCN(S(=O)(=O)CF)CC2)ncc1C#N] Enantiomer 1 (earlier eluting enantiomer), made from cis-1- methylcyclopentane-1,2-diol | LC-MS (ESI): m/z 414.2 [M + H]⁺; ¹H NMR (400 MHz, DMSO-d₆)(tautomer ratio = 1:1) δ 8.51 & 8.44 (s, 1H), 8.27 & 8.08 (d, J = 8.0 Hz, 1H), 5.54 & 5.52 (d, J_{HF} = 46 Hz, 2H), 5.15-5.09 (m, 1H), 4.48 & 4.46 (s, 1H), 3.98-3.90 (m, 1H), 3.72-3.65 (m, 2H), 3.12-3.04 (m, 2H), 2.09- 2.07 (m, 1H), 1.97-1.88 (m, 2H), 1.82-1.74 (m, 3H), 1.60-1.48 (m, 4H), 1.21 & 1.20 (s, 3H). ee: 99.1% Retention time: 2.88 min; Column: ChiralPak IH, 100 × 4.6 mm I.D., 5 μm; Mobile phase: A for CO₂ and B for methanol |
| | | (0.05% DEA); Gradient: 0.0 min- |
| | | 1.0 min @ 10% B, 1.0 min-4.5 |
| | | min gradient (10-40% B), 4.5 |
| | | min-7.0 min @ 40% B, 7.0 min- |
| | | 8.0 min @ 10% B; Flow rate: 2.5 |
| | | mL/min; Column temperature: |
| | | 40° C. |
| 37b | 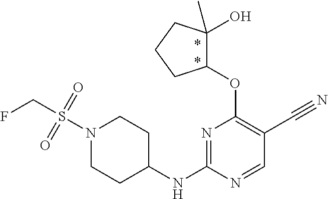 [SMILES: CC1(O)CCCC1Oc1nc(NC2CCN(S(=O)(=O)CF)CC2)ncc1C#N] Enantiomer 2 (later eluting enantiomer), made from cis-1- methylcyclopentane-1,2-diol | LC-MS (ESI): m/z 414.2 [M + H]⁺. ee: 98.7% Retention time: 3.39 min; Column: ChiralPak IH, 100 × 4.6 mm I.D., 5 μm; Mobile phase: A for CO₂ and B for methanol (0.05% DEA); Gradient: 0.0 min- 1.0 min @ 10% B, 1.0 min-4.5 min gradient (10-40% B), 4.5 min-7.0 min @ 40% B, 7.0 min- 8.0 min @ 10% B; Flow rate: 2.5 mL/min; Column temperature: 40° C. |
| 38a | 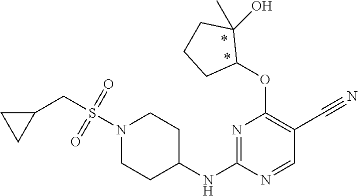 [SMILES: CC1(O)CCCC1Oc1nc(NC2CCN(S(=O)(=O)CC3CC3)CC2)ncc1C#N] Enantiomer 1 (earlier eluting enantiomer), made from cis-1- methylcyclopentane-1,2-diol | LC-MS (ESI): m/z 436.2 [M + H]⁺; ¹H NMR (400 MHz, DMSO-d₆)(tautomer ratio = 1:1) δ 8.51 & 8.44 (s, 1H), 8.26 & 8.04 (d, J = 8.0 Hz, 1H), 5.12-5.08 (m, 1H), 4.47 & 4.46 (s, 1H), 3.97- 3.84 (m, 1H), 3.65-3.58 (m, 2H), 3.02-2.88 (m, 4H), 2.11-2.03 (m, 1H), 1.96-1.85 (m, 2H), 1.79-1.66 (m, 3H), 1.58-1.44 (m, 4H), 1.21 & 1.19 (s, 3H), 1.05-0.93 (m, 1H), 0.62-0.54 (m, 2H), 0.37-0.29 (m, 2H). ee: 99.3% Retention time: 3.34 min, Column: Chiralpak AS-3, 150 × |
| | | 4.6 mm I.D., 3 μm, Mobile phase: |
| | | A: CO₂ B: ethanol (0.05% DEA) |
| | | Gradient: from 5% to 40% of B in |
| | | 5 min and hold 40% for 2.5 min, |
| | | then 5% of B for 2.5 min, Flow |
| | | rate: 2.5 mL/min, Column |
| | | temperature: 35° C. |
| 38b | 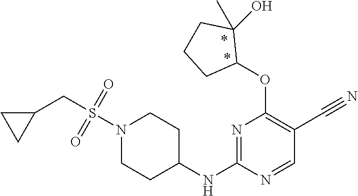 [SMILES: CC1(O)CCCC1Oc1nc(NC2CCN(S(=O)(=O)CC3CC3)CC2)ncc1C#N] Enantiomer 2 (later eluting enantiomer), made from cis-1- methylcyclopentane-1,2-diol | LC-MS (ESI): m/z 436.2 [M + H ]⁺. ee: 98.5% Retention time: 3.75 min; Column: Chiralpak AS-3, 150 × 4.6 mm I.D., 3 μm, Mobile phase: A: CO₂ B: ethanol (0.05% DEA) Gradient: from 5% to 40% of B in 5 min and hold 40% for 2.5 min, then 5% of B for 2.5 min, Flow rate: 2.5 mL/min, Column temperature: 35° C. |
| 39a | 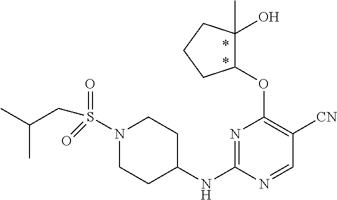 [SMILES: CC(C)CS(=O)(=O)N1CCC(Nc2ncc(C#N)c(OC3CCCC3(C)O)n2)CC1] Enantiomer 1 (earlier eluting enantiomer), made from cis-1- methylcyclopentane-1,2-diol | LC-MS (ESI): m/z 438.2 [M + H]⁺; ¹H NMR (400 MHz, DMSO-d₆)(tautomer ratio = 1:1) δ 8.50 & 8.44 (s, 1H), 8.24 & 8.05 (d, J = 8.0 Hz, 1H), 5.27-4.85 (m, 1H), 4.48 & 4.47 (s, 1H), 3.98- 3.82 (m, 1H), 3.65-3.57 (m, 2H), 2.99-2.79 (m, 4H), 2.24-2.00 (m, 2H), 1.89-1.78 (m, 2H), 1.84-1.67 (m, 3H), 1.57-1.49 (m, 4H), 1.21 & 1.19 (s, 3H), 1.03 (d, J = 6.7 Hz, 6H). ee: 97.3% Retention time: 2.99 min; Column: ChiralPak AS, 250 × 4.6 mm I.D., 5 μm, Mobile phase: A for CO₂ and B for isopropanol |
| | | 0.05% DEA), Gradient: 8 min @ |
| | | 20%, Flow rate: 2.0 mL/min, |
| | | Back pressure: 100 bar, Column |
| | | temperature: 35° C. |
| 39b | 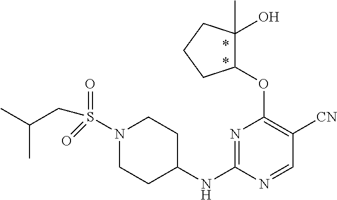 [SMILES: CC(C)CS(=O)(=O)N1CCC(Nc2ncc(C#N)c(OC3CCCC3(C)O)n2)CC1] Enantiomer 2 (later eluting enantiomer), made from cis-1- methylcyclopentane-1,2-diol | LC-MS (ESI): m/z 438.2 [M + H]⁺. ee: 95.9% Retention time: 3.86 min; Column: ChiralPak AS, 250 × 4.6 mm I.D., 5 μm, Mobile phase: A for CO₂ and B for isopropanol (0.05% DEA), Gradient: 8 min @ 20%, Flow rate: 2.0 mL/min, Back pressure: 100 bar, Column temperature: 35° C. |
| 40a | 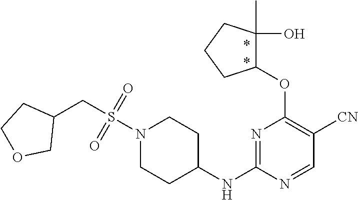 [SMILES: CC1(O)CCCC1Oc1nc(NC2CCN(S(=O)(=O)CC3CCOC3)CC2)ncc1C#N] Enantiomer 1 (earlier eluting enantiomer), made from cis-1- methylcyclopentane-1,2-diol | LC-MS (ESI): m/z 466.3 [M + H]⁺ ; ¹H NMR (400 MHz, DMSO-d₆)(tautomer ratio = 1:1) δ 8.50 & 8.43 (s, 1H), 8.23 & 8.05 (d, J = 8.0 Hz, 1H), 5.13-5.07 (m, 1H), 4.49 & 4.47 (s, 1H), 3.86- 3.82 (m, 2H), 3.73-3.72 (m, 1H), 3.64-3.60 (m, 3H), 3.16-3.14 (m, 2H), 3.14-3.29 (m, 2H), 2.50-2.49 (m, 1H), 2.08-2.06 (m, 2H), 1.76- 1.74 (m, 2H), 1.70-1.51 (m, 9H), 1.20 & 1.19 (s, 3H). ee: 95.5% Retention time: 2.16 & 2.28 min; Column: ChiralPak IH, 100 × 4.6 mm I.D., 5 μm; Mobile phase: A for CO₂ and B for methanol |
| | | (0.05% DEA); Gradient: 8 min @ |
| | | 20% B; Flow rate: 2.5 mL/min; |
| | | Column temperature: 40° C. |
| 40b | 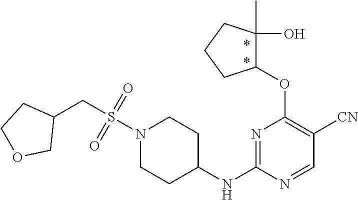 [SMILES: CC1(O)CCCC1Oc1nc(NC2CCN(S(=O)(=O)CC3CCOC3)CC2)ncc1C#N] Enantiomer 2 (later eluting enantiomer), made from cis-1- methylcyclopentane-1,2-diol | LC-MS (ESI): m/z 466.3 [M + H]⁺. ee: 96.9% Retention time: 3.07 & 3.29 min; Column: ChiralPak IH, 100 × 4.6 mm I.D., 5 μm; Mobile phase: A for CO₂ and B for methanol (0.05% DEA); Gradient: 8 min @ 20% B; Flow rate: 2.5 mL/min; Column temperature: 40° C. |
| 41a | 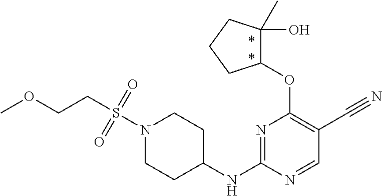 [SMILES: COCCS(=O)(=O)N1CCC(Nc2ncc(C#N)c(OC3CCCC3(C)O)n2)CC1] Enantiomer 1 (earlier eluting enantiomer), made from cis-1- methylcyclopentane-1,2-diol | LC-MS (ESI): m/z 440.2 [M + H]⁺; ¹H NMR (400 MHz, DMSO-d₆)(tautomer ratio = 1:1) δ 8.51 & 8.45 (s, 1H), 8.25 & 8.05 (d, J = 8.0 Hz, 1H), 5.12-5.08 (m, 1H), 4.50 & 4.49 (s, 1H), 3.95- 3.72 (m, 1H), 3.68-3.64 (m, 2H), 3.60-3.49 (m, 2H), 3.32-3.26 (m, 5H), 2.96-2.89 (m, 2H), 2.13-2.08 (m, 1H), 1.97-1.87 (m, 2H), 1.72- 1.67 (m, 3H), 1.59-1.31 (m, 4H), 1.22 & 1.20 (s, 3H). ee: 98.3% Retention time: 2.92 min; Column: ChiralPak IH, 100 × 4.6 mm I.D., 5 μm; Mobile phase: A |
| | | for CO₂ and B for methanol |
| | | (0.05% DEA); Gradient: 0.0 min- |
| | | 1.0 min @ 10% B, 1.0 min-4.5 |
| | | min gradient (10-40% B), 4.5 |
| | | min-7.0 min @ 40% B, 7.0 min- |
| | | 8.0 min @ 10% B; Flow rate: 2.5 |
| | | mL/min; Column temperature: |
| | | 40° C. |
| 41b | 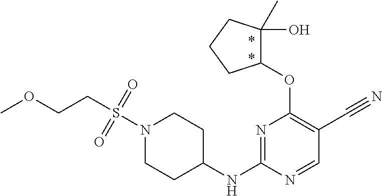 [SMILES: COCCS(=O)(=O)N1CCC(Nc2ncc(C#N)c(OC3CCCC3(C)O)n2)CC1] Enantiomer 2 (later eluting enantiomer), made from cis-1- methylcyclopentan-1,2-diol | LC-MS (ESI): m/z 440.2 [M + H]⁺. ee: 95.9% Retention time: 3.32 min; Column: ChiralPak IH, 100 × 4.6 mm I.D., 5 μm; Mobile phase: A for CO₂ and B for methanol (0.05% DEA); Gradient: 0.0 min- 1.0 min @ 10% B, 1.0 min-4.5 min gradient (10-40% B), 4.5 min-7.0 min @ 40% B, 7.0 min- 8.0 min @ 10% B; Flow rate: 2.5 mL/min; Column temperature: 40° C. |
| 42a | 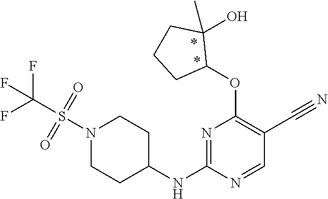 [SMILES: CC1(O)CCCC1Oc1nc(NC2CCN(S(=O)(=O)C(F)(F)F)CC2)ncc1C#N] Enantiomer 1 (earlier eluting enantiomer), made from cis-1- methylcyclopentane-1,2-diol | LC-MS (ESI): m/z 450.2 [M + H]⁺; ¹H NMR (400 MHz, DMSO-d₆)(tautomer ratio = 1:1) δ 8.52 & 8.51 (s, 1H), 8.30 & 8.10 (d, J = 8.0 Hz, 1H), 5.17-5.06 (m, 1H), 4.48 & 4.45 (s, 1H), 4.13- 3.96 (m, 1H), 3.82-3.79 (m, 2H), 3.38-3.34 (m, 2H), 2.09-2.07 (m, 1H), 1.99-1.96 (m, 2H), 1.78-1.72 (m, 3H), 1.60-1.52 (m, 4H), 1.21 & 1.20 (s, 3H). ee: 100% Retention time: 1.72 min; Column: ChiralPak IH, 100 × 4.6 mm I.D., 5 μm; Mobile phase: A for CO₂ and B for methanol |
| | | (0.05% DEA); Gradient: 0.0 min- |
| | | 1.0 min @ 10% B, 1.0 min-4.5 |
| | | min gradient (10-40% B), 4.5 |
| | | min-7.0 min @ 40% B, 7.0 min- |
| | | 8.0 min @ 10% B; Flow rate: 2.5 |
| | | mL/min; Column temperature: |
| | | 40° C. |
| 42b | 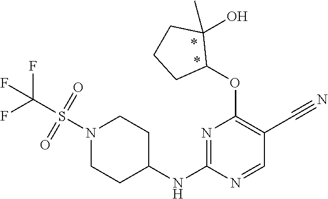 [SMILES: CC1(O)CCCC1Oc1nc(NC2CCN(S(=O)(=O)C(F)(F)F)CC2)ncc1C#N] Enantiomer 2 (later eluting enantiomer), made from cis-1- methylcyclopentane-1,2-diol | LC-MS (ESI): m/z 450.2 [M + H]⁺. ee: 94.3% Retention time: 2.45 min; Column: ChiralPak IH, 100 × 4.6 mm I.D., 5 μm; Mobile phase: A for CO₂ and B for methanol (0.05% DEA); Gradient: 0.0 min- 1.0 min @ 10% B, 1.0 min-4.5 min gradient (10-40% B), 4.5 min-7.0 min @ 40% B, 7.0 min- 8.0 min @ 10% B; Flow rate: 2.5 mL/min; Column temperature: 40° C. |
| 43a | 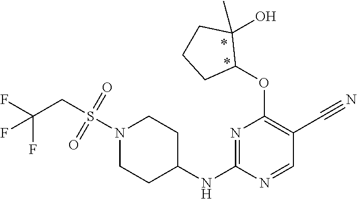 [SMILES: CC1(O)CCCC1Oc1nc(NC2CCN(S(=O)(=O)CC(F)(F)F)CC2)ncc1C#N] Enantiomer 1 (earlier eluting enantiomer), made from cis-1- methylcyclopentane-1,2-diol | LC-MS (ESI): m/z 464.2 [M + H]⁺; ¹H NMR (400 MHz, DMSO-d₆)(tautomer ratio = 1:1) δ 8.52 & 8.45 (s, 1H), 8.26 & 8.06 (d, J = 8.0 Hz, 1H), 5.12-5.09 (m, 1H), 4.55-4.45 (m, 3H), 3.95-3.85 (m, 1H), 3.68-3.62 (m, 2H), 3.01- 2.95 (m, 2H), 2.09-2.07 (m, 1H), 2.05-1.91 (m, 2H), 1.78-1.76 (m, 3H), 1.60-1.54 (m, 4H), 1.21 & 1.20 (s, 3H). ee: 96.0% Retention time: 2.28 min; Column: ChiralPak IH, 100 × 4.6 mm I.D., 5 μm; Mobile phase: A for CO₂ and B for methanol |
| | | (0.05% DEA); Gradient: 0.0 min- |
| | | 1.0 min @ 10% B, 1.0 min-4.5 |
| | | min gradient (10-40% B), 4.5 |
| | | min-7.0 min @ 40% B, 7.0 min- |
| | | 8.0 min @ 10% B; Flow rate: 2.5 |
| | | mL/min; Column temperature: |
| | | 40° C. |
| 43b | 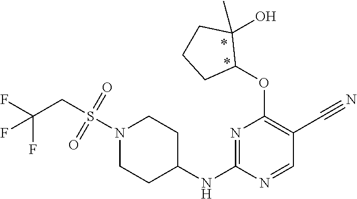 [SMILES: CC1(O)CCCC1Oc1nc(NC2CCN(S(=O)(=O)CC(F)(F)F)CC2)ncc1C#N] Enantiomer 2 (later eluting enantiomer), made from cis-1- methylcyclopentane-1,2-diol | LC-MS (ESI): m/z 464.2 [M + H]⁺. ee: 93.5% Retention time: 2.71 min; Column: ChiralPak IH, 100 × 4.6 mm I.D., 5 μm; Mobile phase: A for CO₂ and B for methanol (0.05% DEA); Gradient: 0.0 min- 1.0 min @ 10% B, 1.0 min-4.5 min gradient (10-40% B), 4.5 min-7.0 min @ 40% B, 7.0 min- 8.0 min @ 10% B; Flow rate: 2.5 mL/min; Column temperature: 40° C. |
| 44a | 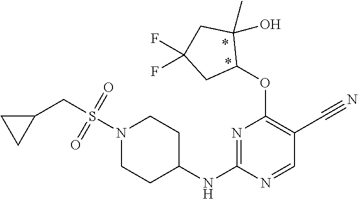 [SMILES: CC1(O)CC(F)(F)CC1Oc1nc(NC2CCN(S(=O)(=O)CC3CC3)CC2)ncc1C#N] Enantiomer 1 (earlier eluting enantiomer), made from cis-4,4- difluoro-1-methylcyclopentane-1,2- diol | LC-MS (ESI): m/z 472.2 [M + H]⁺. ee: 100% Retention time: 1.06 min; Column: Chiralpak AD-3, 150 × 4.6 mm I.D., 3 μm, Mobile phase: 40% of ethanol (0.05% DEA) in CO₂, Flow rate: 2.5 mL/min, Column temperature: 35° C. |
| 44b | 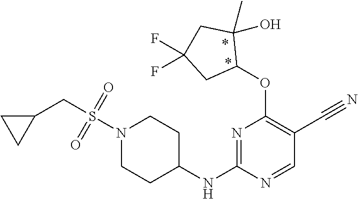 [SMILES: CC1(O)CC(F)(F)CC1Oc1nc(NC2CCN(S(=O)(=O)CC3CC3)CC2)ncc1C#N] Enantiomer 2 (later eluting enantiomer), made from cis-4,4- difluoro-1-methylcyclopentane-1,2- diol | LC-MS (ESI): m/z 472.2 [M + H]⁺; ¹H NMR (400 MHz, DMSO-d₆)(tautomer ratio = 1:1) δ 8.54 & 8.48 (s, 1H), 8.35 & 8.15 (d, J = 8.0 Hz, 1H), 5.34-5.23 (m, 1H), 5.15 & 5.13 (s, 1H), 3.98- 3.80 (m, 1H), 3.63-3.60 (m, 2H), 3.01-2.89 (m, 4H), 2.85-2.63 (m, 1H), 2.44-2.32 (m, 3H), 1.97- 1.81 (m, 2H), 1.64-1.39 (m, 2H), 1.30 & 1.29 (s, 3H), 0.99 (s, 1H), 0.61-0.54 (m, 2H), 0.36-0.31 (m, 2H). ee: 100% Retention time: 1.31 min; Column: Chiralpak AD-3, 150 × 4.6 mm I.D., 3 μm, Mobile phase: |
| | | 40% of ethanol (0.05% DEA) in |
| | | CO₂, Flow rate: 2.5 mL/min, |
| | | Column temperature: 35° C. |
| 45a | 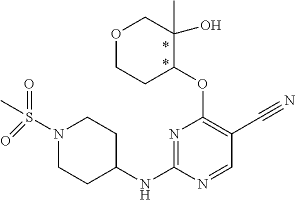 [SMILES: CC1(O)COCCC1Oc1nc(NC2CCN(S(C)(=O)=O)CC2)ncc1C#N] Enantiomer 1 (earlier eluting enantiomer), made form cis-3- methyltetrahydro-2H-pyran-3,4- diol | LC-MS (ESI): m/z 412.2 [M + H]⁺; ¹H NMR (400 MHz, DMSO-d₆)(tautomer ratio = 1:1) δ 8.54 & 8.47 (s, 1H), 8.31 & 8.12 (d, J = 8.0 Hz, 1H), 5.18-5.16 (m, 1H), 4.73 & 4.71 (s, 1H), 3.95- 3.74 (m, 2H), 3.55-3.47 (m, 4H), 3.30-3.22 (m, 1H), 2.97-2.78 (m, 5H), 2.12-1.85 (m, 4H), 1.68-1.46 (m, 2H), 1.11 (s, 3H). ee: 99.3% Retention time: 3.83 min; Column: ChiralPak IH, 100 × 4.6 mm I.D., 5 μm; Mobile phase: A for CO₂ and B for ethanol (0.05% DEA); Gradient: 0.0 min-1.0 min @ 10% B, 1.0 min-4.5 min |
| | | gradient (10-40% B), 4.5 min- |
| | | 7.0 min @ 40% B, 7.0 min-8.0 |
| | | min @ 10% B; Flow rate: 2.5 |
| | | mL/min; Column temperature: |
| | | 40° C. |
| 45b | 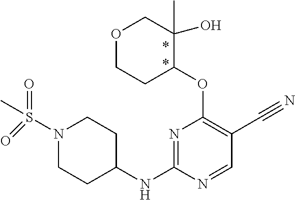 [SMILES: CC1(O)COCCC1Oc1nc(NC2CCN(S(C)(=O)=O)CC2)ncc1C#N] Enantiomer 2 (later eluting enantiomer), made from cis-3- methyltetrahydro-2H-pyran-3,4- diol | LC-MS (ESI): m/z 412.2 [M + H]⁺. ee: 97.0% Retention time: 4.26 min; Column: ChiralPak IH, 100 × 4.6 mm I.D., 5 μm; Mobile phase: A for CO₂ and B for ethanol (0.05% DEA); Gradient: 0.0 min-1.0 min @ 10% B, 1.0 min-4.5 min gradient (10-40% B), 4.5 min-7.0 min @ 40% B, 7.0 min-8.0 min @ 10% B; Flow rate: 2.5 mL/min; Column temperature: 40° C. |
| 46a | 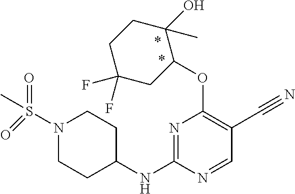 [SMILES: CC1(O)CCC(F)(F)CC1Oc1nc(NC2CCN(S(C)(=O)=O)CC2)ncc1C#N] Enantiomer 1 (earlier eluting enantiomer), made form cis-4,4- difluoro-1-methylcyclohexane-1,2- diol | LC-MS (ESI): m/z 446.2 [M + H]⁺. ee: 100% Retention time: 4.66 min; Column: Cellulose-2, 150 × 4.6 mm I.D., 3 μm, Mobile phase: A: CO₂ B: ethanol (0.05% DEA), Gradient: from 5% to 40% of B in 5 min and hold 40% for 2.5 min, then 5% of B for 2.5 min, Flow rate: 2.5 mL/min, Column temperature: 35° C. |
| 46b | 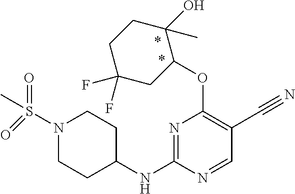 [SMILES: CC1(O)CCC(F)(F)CC1Oc1nc(NC2CCN(S(C)(=O)=O)CC2)ncc1C#N] Enantiomer 2 (later eluting enantiomer), made from cis-4,4- difluoro-1-methylcyclohexane-1,2- diol | LC-MS (ESI): m/z 446.2 [M + H]⁺; ¹H NMR (400 MHz, DMSO-d₆)(tautomer ratio = 1:1) δ 8.56 & 8.50 (s, 1H), 8.40 & 8.15 (d, J = 8.0 Hz, 1H), 5.18-5.14 (m, 1H), 4.93 & 4.90 (s, 1H), 3.98- 3.72 (m, 1H), 3.60-3.51 (m, 2H), 2.87 (s, 3H), 2.82-2.71 (m, 2H), 2.37-1.86 (m, 6H), 1.74-1.68 (m, 1H), 1.67-1.46 (m, 3H), 1.21 & 1.15 (s, 3H). ee: 98.5% Retention time: 4.96 min; Column: Cellulose-2, 150 × 4.6 mm I.D., 3 μm, Mobile phase: A: CO₂ B: ethanol (0.05% DEA), Gradient: from 5% to 40% of B in |
| | | 5 min and hold 40% for 2.5 min, |
| | | then 5% of B for 2.5 min, Flow |
| | | rate: 2.5 mL/min, Column |
| | | temperature: 35° C. |
| 47a | 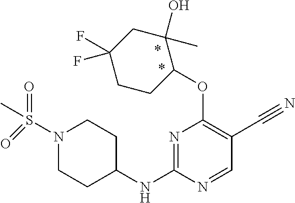 [SMILES: CC1(O)CC(F)(F)CCC1Oc1nc(NC2CCN(S(C)(=O)=O)CC2)ncc1C#N] Enantiomer 1 (earlier eluting enantiomer), made from cis-5,5- difluoro-1-methylcyclohexane-1,2- diol | LC-MS (ESI): m/z 446.2 [M + H]⁺; ¹H NMR (400 MHz, DMSO-d₆)(tautomer ratio = 1:1) δ 8.63 & 8.46 (s, 1H), 8.31 & 8.13 (d, J = 8.0 Hz, 1H), 5.20-5.17 (m, 1H), 4.94 & 4.86 (s, 1H), 3.95- 3.84 (m, 1H), 3.51-3.48 (m, 2H), 2.87-2.85 (m, 5H), 2.06-1.86 (m, 8H), 1.58-1.52 (m, 2H), 1.21 (s, 3H). ee: 100% Retention time: 3.35 min; Column: ChiralPak IG, 250 × 4.6 mm I.D., 5 μm, Mobile phase: A for CO₂ and B for MeOH (0.05% DEA), Gradient: 8 min @ B 40%, Flow rate: 2.0 mL/min, Back pressure: 100 bar, Column |
| | | temperature: 35° C. |
| 47b | 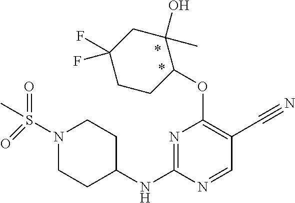 [SMILES: CC1(O)CC(F)(F)CCC1Oc1nc(NC2CCN(S(C)(=O)=O)CC2)ncc1C#N] Enantiomer 2 (later eluting enantiomer), made from cis-5,5- difluoro-1-methylcyclohexane-1,2- diol | LC-MS (ESI): m/z 446.2 [M + H]⁺. ee: 100% Retention time: 11.42 min; Column: ChiralPak IG, 250 × 4.6 mm I.D., 5 μm, Mobile phase: A for CO₂ and B for MeOH (0.05% DEA), Gradient: 8 min @ B 40%, Flow rate: 2.0 mL/min, Back pressure: 100 bar, Column temperature: 35° C. |
| 48a | 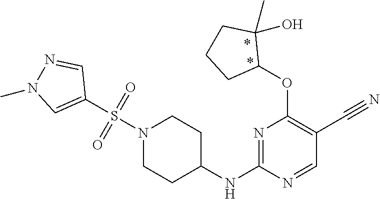 [SMILES: Cn1cc(S(=O)(=O)N2CCC(Nc3ncc(C#N)c(OC4CCCC4(C)O)n3)CC2)cn1] Enantiomer 1 (earlier eluting enantiomer), made from cis-1- methylcyclopentane-1,2-diol | LC-MS (ESI): m/z 462.2 [M + H]⁺; ¹H NMR (400 MHz, DMSO-d₆)(tautomer ratio = 1:1) δ 8.46 & 8.42 (s, 1H), 8.33 & 8.31 (s, 1H), 8.21 & 8.01 (d, J = 8.0 Hz, 1H), 7.78 & 7.76 (s, 1H), 5.10- 5.05 (m, 1H), 4.47 & 4.43 (s, 1H), 3.90 (s, 3H), 3.79-3.68 (m, 1H), 3.54-3.42 (m, 2H), 2.42-2.36 (m, 2H), 2.06-2.04 (m, 1H), 1.92-1.90 (m, 2H), 1.75-1.73 (m, 3H), 1.60- 1.53 (m, 4H), 1.18 & 1.16 (s, 3H). ee: 99.1% Retention time: 1.05 min; Column: Chiralpak AS-3, 150 × 4.6 mm I.D., 3 μm, Mobile phase: |
| | | 40% of ethanol (0.05% DEA) in |
| | | CO₂, Flow rate: 2.5 mL/min, |
| | | Column temperature: 35° C. |
| 48b | 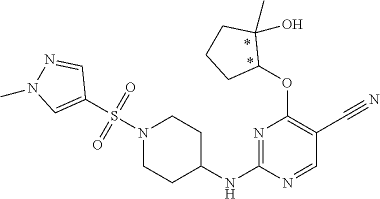 [SMILES: Cn1cc(S(=O)(=O)N2CCC(Nc3ncc(C#N)c(OC4CCCC4(C)O)n3)CC2)cn1] Enantiomer 2 (later eluting enantiomer), made from cis-1- methylcyclopentane-1,2-diol | LC-MS (ESI): m/z 462.2 [M + H]⁺. ee: 100% Retention time: 1.24 min; Column: Chiralpak AS-3, 150 × 4.6 mm I.D., 3 μm, Mobile phase: 40% of ethanol (0.05% DEA) in CO₂, Flow rate: 2.5 mL/min, Column temperature: 35° C. |
| 49a | 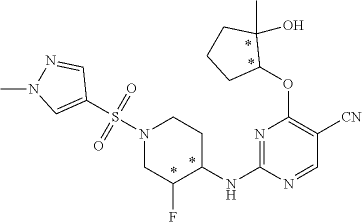 [SMILES: Cn1cc(S(=O)(=O)N2CCC(Nc3ncc(C#N)c(OC4CCCC4(C)O)n3)C(F)C2)cn1] Isomer 1 (1st eluting isomer), made from cis-1-methylcyclopentane- 1,2-diol and cis-3-fluoropiperidin- 4-amine | LC-MS (ESI): m/z 480.2 [M + H]⁺; ¹H NMR (400 MHz, DMSO-d₆)(tautomer ratio = 1:1) δ 8.49 & 8.46 (s, 1H), 8.35 & 8.31 (s, 1H), 8.30 & 8.15 (d, J = 8.0 Hz, 1H), 7.80 & 7.77 (s, 1H), 5.16- 5.12 (m, 1H, 5.11-4.82 (m, 1H), 4.48 & 4.47 (s, 1H), 4.07-3.94 (m, 1H), 3.90 (s, 3H), 3.84-3.78 (m, 1H), 3.62-3.58 (m, 1H), 2.74-2.56 (m, 2H), 2.07-1.93 (m, 2H), 1.75- 1.70 (m, 4H), 1.50-1.51 (m, 2H), 1.19 & 1.13 (s, 3H). ee: 93.4% Retention time: 4.80 min; Column: ChiralPak IH, 100 × 4.6 mm I.D., 5 μm; Mobile phase: A for CO₂ and B for Isopropyl alcohol (0.05% DEA); Gradient: |
| | | 0.0 min-1.0 min @ 10% B, 1.0 |
| | | min-4.5 min gradient (10-40% B), |
| | | 4.5 min-7.0 min @ 40% B, 7.0 |
| | | min-8.0 min @ 10% B; Flow rate: |
| | | 2.5 mL/min; Column |
| | | temperature: 40° C. |
| 49b | 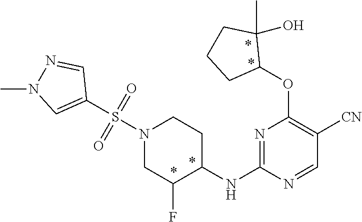 [SMILES: Cn1cc(S(=O)(=O)N2CCC(Nc3ncc(C#N)c(OC4CCCC4(C)O)n3)C(F)C2)cn1] Isomer 2 (2nd eluting isomer), made from cis-1-methylcyclopentane- 1,2-diol and cis-3-fluoropiperidin- 4-amine | LC-MS (ESI): m/z 480.2 [M + H]⁺. ee: 89.4% Retention time: 5.29 min; Column: ChiralPak IH, 100 × 4.6 mm I.D., 5 μm; Mobile phase: A for CO₂ and B for Isopropyl alcohol (0.05% DEA); Gradient: 0.0 min-1.0 min @ 10% B, 1.0 min-4.5 min gradient (10-40% B), 4.5 min-7.0 min @ 40% B, 7.0 min-8.0 min @ 10% B; Flow rate: 2.5 mL/min; Column temperature: 40° C. |
| 49c | 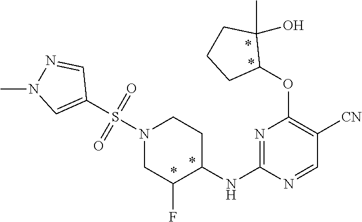 [SMILES: Cn1cc(S(=O)(=O)N2CCC(Nc3ncc(C#N)c(OC4CCCC4(C)O)n3)C(F)C2)cn1] Isomer 3 (3rd eluting isomer), made from cis-1-methylcyclopentane- 1,2-diol and cis-3-fluoropiperidin- 4-amine | LC-MS (EIS): m/z 480.2 [M + H]⁺; ¹H NMR (400 MHz, DMSO-d₆)(tautomer ratio = 1:1) δ 8.48 & 8.46 (s, 1H), 8.35 & 8.31 (s, 1H), 8.30 & 8.15 (d, J = 8.0 Hz, 1H), 7.81 & 7.77 (s, 1H), 5.16- 5.12 (m, 1H), 5.11-4.82 (m, 1H), 4.49 & 4.41 (s, 1H), 4.07-3.94 (m, 1H), 3.90 (s, 3H), 3.84-3.78 (m, 1H), 3.62-3.58 (m, 1H), 2.74-2.56 (m, 2H), 2.07-1.93 (m, 2H), 1.75- 1.70 (m, 4H), 1.56-1.50 (m, 2H), 1.20 & 1.14 (s, 3H). ee: 86.2% Retention time: 5.73 min; Column: ChiralPak IH, 100 × 4.6 mm I.D., 5 μm; Mobile phase: A form CO₂ and B for Isopropyl alcohol (0.05% DEA); Gradient: |
| | | 0.0 min-1.0 min @ 10% B, 1.0 |
| | | min-4.5 min gradient (10-40% B), |
| | | 4.5 min-7.0 min @ 40% B, 7.0 |
| | | min-8.0 min @ 10% B; Flow rate: |
| | | 2.5 mL/min; Column |
| | | temperature: 40° C. |
| 49d | 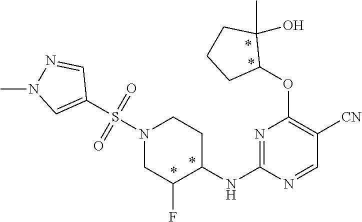 [SMILES: Cn1cc(S(=O)(=O)N2CCC(Nc3ncc(C#N)c(OC4CCCC4(C)O)n3)C(F)C2)cn1] Isomer 4 (4th eluting isomer), made from cis-1-methylcyclopentane- 1,2-diol and cis-3-fluoropiperidin- 4-amine | LC-MS (ESI): m/z 480.2 [M + H]⁺. ee: 94.0% Retention time: 6.38 min; Column: ChiralPak IH, 100 × 4.6 mm I.D., 5 μm; Mobile phase: A for CO₂ and B for Isopropyl alcohol (0.05% DEA); Gradient: 0.0 min-1.0 min @ 10% B, 1.0 min-4.5 min gradient (10-40% B), 4.5 min-7.0 min @ 40% B, 7.0 min-8.0 min @ 10% B; Flow rate: 2.5 mL/min; Column temperature: 40° C. |
| 50a | 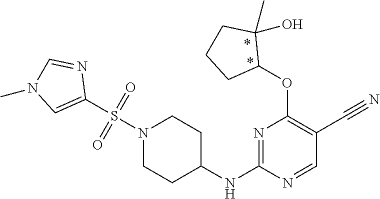 [SMILES: Cn1cnc(S(=O)(=O)N2CCC(Nc3ncc(C#N)c(OC4CCCC4(C)O)n3)CC2)c1] Enantiomer 1 (earlier eluting enantiomer), made from cis-1- methylcyclopentane-1,2-diol | LC-MS (ESI): m/z 462.3 [M + H]⁺; ¹H NMR (400 MHz, DMSO)(tautomer ratio = 1:1) @ 8.47 & 8.42 (s, 1H), 8.19 & 8.12 (d, J = 8.0 Hz, 1H), 7.82 & 7.80 (s, 2H), 5.09-5.05 (m, 1H), 4.46- 4.43 (m, 1H), 3.72 (s, 1H), 3.71 (s, 3H), 3.58-3.56 (m, 2H), 2.67-2.57 (m, 2H), 1.91-1.88 (m, 1H), 1.76- 1.75 (m, 2H), 1.56-1.53 (m, 3H), 1.52-1.48 (m, 4H), 1.18 & 1.16 (s, 3H). ee: 100% Retention time: 7.92 min; Column: ChiralPak IG, 250 × 4.6 mm I.D., 5 um, Mobile phase: A |
| | | for CO₂ and B for MeOH (0.05% |
| | | DEA), Gradient: 8 min @ B 40%, |
| | | Flow rate: 2.0 mL/min, Back |
| | | pressure: 100 bar, Column |
| | | temperature: 35° C. |
| 50b | 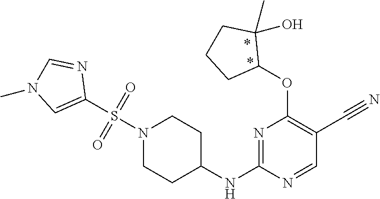 [SMILES: Cn1cnc(S(=O)(=O)N2CCC(Nc3ncc(C#N)c(OC4CCCC4(C)O)n3)CC2)c1] Enantiomer 2 (later eluting enantiomer), made from cis-1- methylcyclopentane-1,2-diol | LC-MS (ESI): m/z 462.3 [M + H]⁺. ee: 97.5% Retention time: 10.35 min; Column: ChiralPak IG, 250 × 4.6 mm I.D., 5 μm, Mobile phase: A for CO₂ and B for MeOH (0.05% DEA), Gradient: 8 min @ B 40%, Flow rate: 2.0 mL/min, Back pressure: 100 bar, Column temperature: 35° C. |
| 51a | 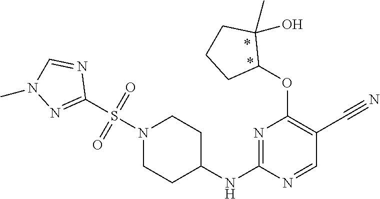 [SMILES: Cn1cnc(S(=O)(=O)N2CCC(Nc3ncc(C#N)c(OC4CCCC4(C)O)n3)CC2)n1] Enantiomer 1 (earlier eluting enantiomer), made from cis-1- methylcyclopentane-1,2-diol | LC-MS (ESI): m/z 463.3 [M + H]⁺; ¹H NMR (400 MHz, DMSO-d₆)(tautomer ratio = 1:1) δ 8.77 & 8.76 (s, 1H), 8.48 & 8.43 (s, 1H), 8.23 & 8.04 (d, J = 8.0 Hz, 1H), 5.11-5.06 (m, 1H), 4.47 & 4.43 (s, 1H), 3.97 (s, 3H), 3.88- 3.75 (m, 1H), 3.70-3.64 (m, 2H), 2.87-2.79 (m, 2H), 2.07-1.93 (m, 1H), 1.90-1.77 (m, 2H), 1.75-1.64 (m, 3H), 1.57-1.48 (m, 4H), 1.19 & 1.18 (s, 3H). ee: 98.1% Retention time: 1.47 min; Column: ChiralPak IH, 100 × 4.6 mm I.D., 5 μm; Mobile phase: A |
| | | for CO₂ and B for methanol |
| | | (0.05% DEA), Gradient: 8 min @ |
| | | 30% B; Flow rate: 2.5 mL/min; |
| | | Column temperature: 40° C. |
| 51b | 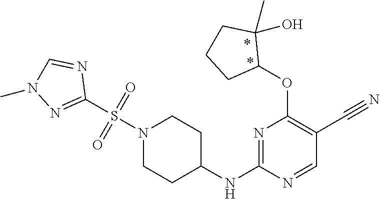 [SMILES: Cn1cnc(S(=O)(=O)N2CCC(Nc3ncc(C#N)c(OC4CCCC4(C)O)n3)CC2)n1] Enantiomer 2 (later eluting enantiomer), made from cis-1- methylcyclopentane-1,2-diol | LC-MS (ESI): m/z 463.3 [M + H]⁺. ee: 95.1% Retention time: 2.10 min; Column: ChiralPak IH, 100 × 4.6 mm I.D., 5 μm; Mobile phase: A for CO₂ and B for methanol (0.05% DEA), Gradient: 8 min @ 30% B; Flow rate: 2.5 mL/min; Column temperature: 40° C. |
| 52 | 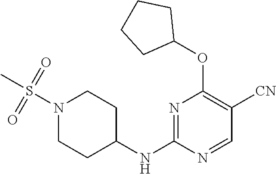 [SMILES: CS(=O)(=O)N1CCC(Nc2ncc(C#N)c(OC3CCCC3)n2)CC1] | LC-MS (ESI): m/z 366.2 [M + H]⁺; ¹H NMR (500 MHz, DMSO-d₆) δ 8.51 & 8.45 (s, 1H), 8.30 & 8.13 (d, J = 7.5 Hz, 1H), 5.44-5.47 (m, 1H), 3.96-3.84 (m, 1H), 3.56-3.52 (m, 2H), 2.90- 2.81 (m, 5H), 2.03-1.87 (m, 4H), 1.81-1.67 (m, 4H), 1.61- 1.63 (m, 4H). |
| 53 | 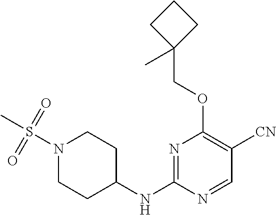 [SMILES: CC1(COc2nc(NC3CCN(S(C)(=O)=O)CC3)ncc2C#N)CCC1] | LC-MS (ESI): m/z 380.2 [M + H]⁺; ¹H NMR (500 MHz, DMSO-d₆) δ 8.55 & 8.48 (s, 1H), 8.34 & 8.20 (d, J = 7.5 Hz, 1H), 4.27 (s, 2H), 4.02-3.84 (m, 1H), 3.57-3.51 (m, 2H), 2.94-2.81 (m, 5H), 2.01-1.83 (m, 6H), 1.77- 1.69 (m, 2H), 1.61-1.51 (m, 2H), 1.19 & 1.18 (s, 3H). |
| 54 | 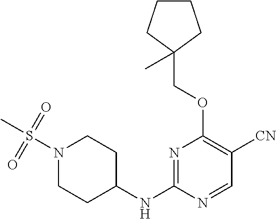 [SMILES: CC1(COc2nc(NC3CCN(S(C)(=O)=O)CC3)ncc2C#N)CCCC1] | LC-MS (ESI): m/z 394.2 [M + H]⁺; ¹H NMR (500 MHz, DMSO-d₆) δ 8.55 & 8.48 (s, 1H), 8.34 & 8.20 (d, J = 7.5 Hz, 1H), 4.20 & 4.13 (s, 2H), 4.01-3.78 (m, 1H), 3.56-3.50 (m, 2H), 2.92- 2.80 (m, 5H), 1.97-1.88 (m, 2H), 1.69-1.51 (m, 8H), 1.38- 1.32 (m, 2H), 1.06 (s, 3H). |
| 55 | 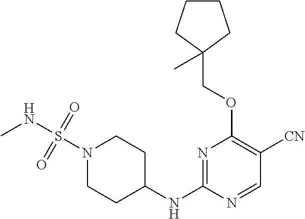 [SMILES: CNS(=O)(=O)N1CCC(Nc2ncc(C#N)c(OCC3(C)CCCC3)n2)CC1] | LC-MS (ESI): m/z 409.2 [M + H]⁺; ¹H NMR (500 MHz, DMSO-d₆) δ 8.53 & 8.46 (s, 1H), 83.1 & 8.16 (d, J = 7.6 Hz, 1H), 7.10-7.02 (m, 1H), 4.21 & 4.13 (s, 2H), 3.99-3.78 (m, 1H), 3.53- 3.47 (m, 2H), 2.86-2.75 (m, 2H), 2.53-2.51 (m, 3H), 1.94-1.85 (m, 2H), 1.71-1.43 (m, 8H), 1.38- 1.32 (m, 2H), 1.06 & 1.05 (s, 3H). |
| 56 | 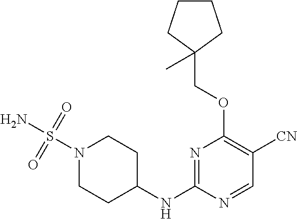 [SMILES: CC1(COc2nc(NC3CCN(S(N)(=O)=O)CC3)ncc2C#N)CCCC1] | LC-MS (ESI): m/z 395.2 [M + H]⁺; ¹H NMR (500 MHz, DMSO-d₆) δ 8.53 & 8.47 (s, 1H), 8.34 & 8.18 (d, J = 7.5 Hz, 1H), 6.75 (s, 2H), 4.21 & 4.13 (s, 2H), 3.89-3.73 (m, 1H), 3.45 (d, J = 12.0 Hz, 2H), 2.72-2.54 (m, 2H), 2.01-1.84 (m, 2H), 1.71-1.46 (m, 8H), 1.42-1.30 (m, 2H), 1.06 & 1.05 (s, 3H). |
| 57 | 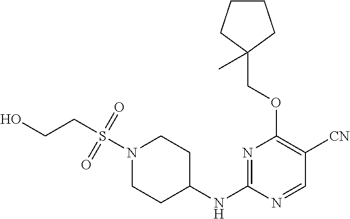 [SMILES: CC1(COc2nc(NC3CCN(S(=O)(=O)CCO)CC3)ncc2C#N)CCCC1] | LC-MS (ESI): m/z 424.2 [M + H]⁺; ¹H NMR (500 MHz, DMSO-d₆) δ 8.53 & 8.46 (s, 1H), 8.32 & 8.18 (d, J = 7.8 Hz, 1H), 5.02 (d, J = 6.0 Hz, 1H), 4.21 & 4.13 (s, 2H), 4.01-3.82 (m, 1H), 3.79-3.69 (m, 2H), 3.62-3.50 (m, 2H), 3.22-3.14 (m, 2H), 3.00- 2.88 (m, 2H), 1.97-1.84 (m, 2H), 1.68-1.58 (m, 4H), 1.58- 1.48 (m, 4H), 1.41-1.31 (m, 2H), 1.06 & 1.05 (s, 3H). |
| 58 | 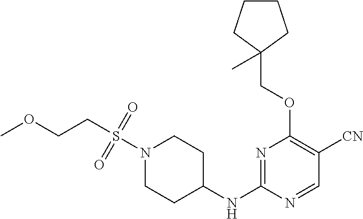 [SMILES: COCCS(=O)(=O)N1CCC(Nc2ncc(C#N)c(OCC3(C)CCCC3)n2)CC1] | LC-MS (ESI): m/z 438.2 [M + H]⁺; ¹H NMR (500 MHz, DMSO-d₆) δ 8.53 & 8.47 (s, 1H), 8.35 & 8.19 (d, J = 7.5 Hz, 1H), 5.75 (s, 2H), 4.21 & 4.13 (s, 2H), 3.96-3.90 (m, 1H), 3.65 (t, J = 6.0 Hz, 2H), 3.59-3.53 (m, 2H), 3.35-3.25 (m, 5H), 1.94-1.85 (m, 2H), 1.71-1.45 (m, 8H), 1.41- 1.32 (m, 2H), 1.05 (s, 3H). |
| 59 | 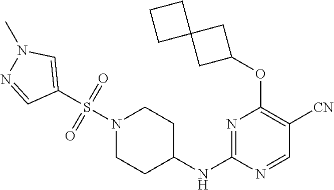 [SMILES: Cn1cc(S(=O)(=O)N2CCC(Nc3ncc(C#N)c(OC4CC5(CCC5)C4)n3)CC2)cn1] | LC-MS (EIS): m/z 458.2 [M + H]⁺; ¹H NMR (500 MHz, DMSO-d₆) δ 8.48 & 8.43 (s, 1H), 8.33 & 8.10 (d, J = 7.5 Hz, 1H), 7.96-7.95 (m, 1H), 6.74 & 6.58 (d, J = 2.5 Hz, 1H), 5.12-4.99 (m, 1H), 3.96 & 3.94 (s, 3H), 3.81- 3.65 (m, 1H), 3.64-3.60 (m, 2H), 2.67-2.52 (m, 3H), 2.48-2.44 (m, 1H), 2.11-1.80 (m, 10H), 1.61-1.51 (m, 2H). |
| 60 | 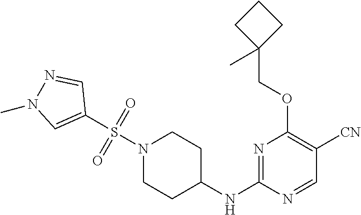 [SMILES: Cn1cc(S(=O)(=O)N2CCC(Nc3ncc(C#N)c(OCC4(C)CCC4)n3)CC2)cn1] | LC-MS (ESI): m/z 446.2 [M + H]⁺; ¹H NMR (500 MHz, DMSO-d₆) δ 8.50 & 8.45 (s, 1H), 8.30 & 8.18 (d, J = 7.5 Hz, 1H), 7.95 (s, 1H), 6.65 (d, J = 2.3 Hz, 1H), 4.26 & 4.21 (s, 2H), 3.96 & 3.94 (s, 3H), 3.88-3.74 (m, 1H), 3.61-3.52 (m, 2H), 2.75-2.54 (m, 2H), 1.99-1.83 (m, 6H), 1.76- 1.67 (m, 2H), 1.62-1.50 (m, 2H), 1.16 & 1.15 (s, 3H). |
| 61 | 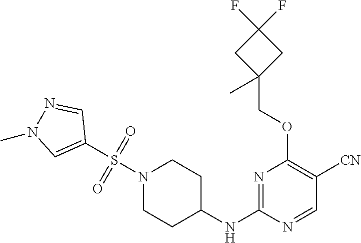 [SMILES: Cn1cc(S(=O)(=O)N2CCC(Nc3ncc(C#N)c(OCC4(C)CC(F)(F)C4)n3)CC2)cn1] | LC-MS (ESI): m/z 482.2 [M + H]⁺; ¹H NMR (500 MHz, DMSO-d₆) δ 8.52 & 8.47 (s, 1H), 8.33 & 8.20 (d, J = 7.5 Hz, 1H), 7.95 (s, 1H), 6.65 (s, 1H), 4.36 & 4.29 (s, 2H), 3.95 & 3.94 (s, 3H), 3.85-3.73 (m, 1H), 3.62-3.56 (m, 2H), 2.73-2.55 (m, 4H), 2.41- 2.28 (m, 2H), 1.95-1.83 (m, 2H), 1.63-1.49 (m, 2H), 1.28 & 1.27 (s, 3H). |
| 62 | 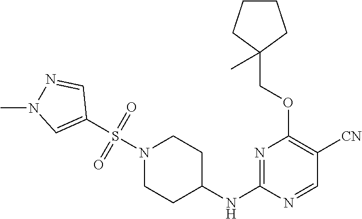 [SMILES: Cn1cc(S(=O)(=O)N2CCC(Nc3ncc(C#N)c(OCC4(C)CCCC4)n3)CC2)cn1] | LC-MS (ESI): m/z 460.2 [M + H]⁺; ¹H NMR (500 MHz, DMSO-d₆) δ 8.49 & 8.44 (s, 1H), 8.28 & 8.15 (d, J = 7.5 Hz, 1H), 7.95 (d, J = 2.5 Hz, 1H), 6.66 (d, J = 2.5 Hz, 1H), 4.14 (d, J = 21.2 Hz, 2H), 3.95 (s, 3H), 3.86-3.69 (m, 1H), 3.62-3.54 (m, 2H), 2.68- 2.52 (m, 2H), 1.95-1.84 (m, 2H), 1.65-1.50 (m, 8H), 1.38- 1.31 (m, 2H), 1.05 & 1.04 (s, 3H). |
| 63 | 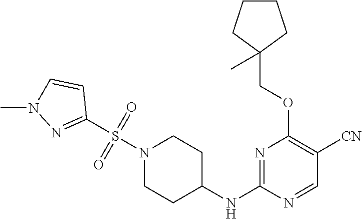 [SMILES: Cn1ccc(S(=O)(=O)N2CCC(Nc3ncc(C#N)c(OCC4(C)CCCC4)n3)CC2)n1] | LC-MS (ESI): m/z 460.2 [M + H ]⁺; ¹H NMR (500 MHz, DMSO-d₆) δ 8.44 & 8.50 (s, 1H), 8.30 & 8.29 (d, J = 7.5 Hz, 1H), 7.96 (t, J = 3.0 Hz, 1H), 6.67 & 6.66 (d, J = 2.5 Hz, 1H), 4.17 & 4.13 (s, 2H), 3.96 & 3.95 (s, 3H), 3.86-3.71 (m, 1H), 3.66-3.54 (m, 2H), 2.55-2.69 (m, 2H), 1.84-1.96 (m, 2H), 1.69-1.50 (m, 8H), 1.41-1.21 (m, 5H), 1.05 (s, 3H). |
| 64a | 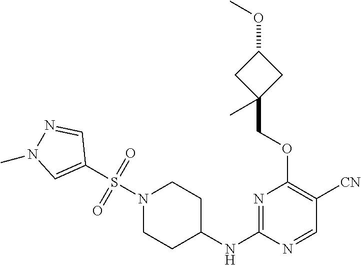 | LC-MS (ESI): m/z 476.2 [M + H]⁺; ¹H NMR (500 MHz, CDCl₃) δ 8.27 (s, 1H), 7.45 (s, 1H), 6.66 (s, 1H), 5.59 & 5.27 (d, J = 7.5 Hz, 1H), 4.16 & 4.13 (s, 2H), 4.00 (s, 3H), 3.97-3.83 (m, 3H), 3.65 (d, J = 12.0 Hz, 1H), 3.22 (s, 3H), 2.95 (t, J = 11.0 Hz, 1H), 2.71 (t, J = 12.0 Hz, 1H), 2.45-2.27 (m, 2H), 2.19-2.03 (m, 2H), 1.88-1.73 (m, 2H), 1.72- 1.57 (m, 2H), 1.24 (s, 3H). |
| 64b | 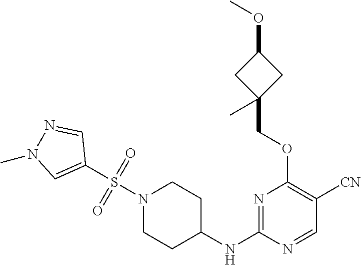 | LC-MS (ESI): m/z 467.2 [M + H]⁺; ¹H NMR (500 MHz, CDCl₃) δ 8.27 & 8.24 (s, 1H), 7.45 (s, 1H), 6.66 (s, 1H), 5.57- 5.30 (d, J = 8.0 Hz, 1H), 4.20 & 4.17 (s, 2H), 4.00 (s, 3H), 3.98- 3.93 (m, 1H), 3.90-3.79 (m, 2H), 3.75-3.65 (m, 1H), 3.21 (s, 3H), 2.89 (t, J = 11.5 Hz, 1H), 2.72 (t, J = 12.0 Hz, 1H), 2.18-2.02 (m, 4H), 1.92-1.82 (m, 2H), 1.75- 1.53 (m, 2H), 1.24 (s, 3H). |
| 67 | 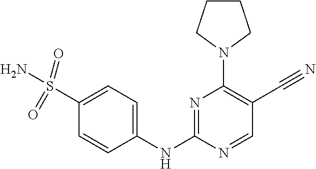 [SMILES: N#Cc1cnc(Nc2ccc(S(N)(=O)=O)cc2)nc1N1CCCC1] | LC-MS (ESI): m/z 345.1 [M + H]⁺; ¹H NMR (400 MHz, DMSO-d₆) δ 10.14 (s, 1H), 8.42 (s, 1H), 7.92 (d, J = 8.8 Hz, 2H), 7.75 (d, J = 8.8 Hz, 2H), 7.19 (s, 2H), 3.74 (br, 4H), 1.96 (br, 4H). |
| 68 | 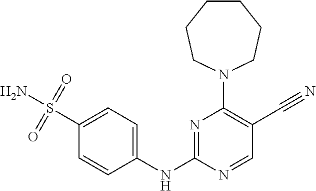 [SMILES: N#Cc1cnc(Nc2ccc(S(N)(=O)=O)cc2)nc1N1CCCCCC1] | LC-MS (ESI): m/z 373.1 [M + H]⁺; ¹H NMR (400 MHz, DMSO-d₆) δ 10.14 (s, 1H), 8.43 (s, 1H), 7.87 (d, J = 8.8 Hz, 2H), 7.75 (d, J = 8.8 Hz, 2H), 7.20 (s, 2H), 3.86 (t, J = 5.6 Hz, 4H), 1.81 (br, 4H), 1.53 (br, 4H). |
| 69 | 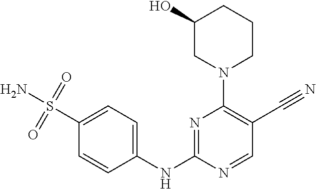 [SMILES: N#Cc1cnc(Nc2ccc(S(N)(=O)=O)cc2)nc1N1CCC[C@H](O)C1] | LC-MS (ESI): m/z 375.1 [M + H]⁺; ¹H NMR (400 MHz, DMSO-d₆) δ 10.18 (br s, 1H), 8.46 (s, 1H), 7.86 (d, J = 8.8 Hz, 2H), 7.76 (d, J = 8.8 Hz, 2H), 7.22 (s, 2H), 5.04 (d, J = 4.6 Hz, 1H), 4.23-4.19 (m, 1H), 4.05-4.02 (m, 1H), 3.64-3.54 (m, 3H), 1.90-1.88 (m, 2H), 1.54-1.42 (m, 2H). |
| 70 | 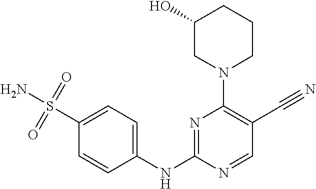 [SMILES: N#Cc1cnc(Nc2ccc(S(N)(=O)=O)cc2)nc1N1CCC[C@@H](O)C1] | LC-MS (ESI): m/z 375.1 [M + H]⁺; ¹H NMR (400 MHz, DMSO-d₆) δ 10.16 (br s, 1H), 8.45 (s, 1H), 7.85 (d, J = 8.8 Hz, 2H), 7.75 (d, J = 8.8 Hz, 2H), 7.22 (s, 2H), 5.05 (d, J = 4.6 Hz, 1H), 4.21-4.18 (m, 1H), 4.04-4.00 (m, 1H), 3.65-3.54 (m, 3H), 1.90-1.87 (m, 2H), 1.53-1.49 (m, 2H). |
| 71 | 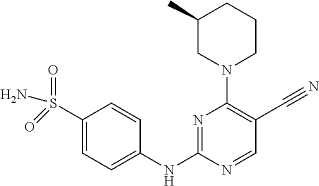 [SMILES: C[C@H]1CCCN(c2nc(Nc3ccc(S(N)(=O)=O)cc3)ncc2C#N)C1] | LC-MS (ESI): m/z 373.1 [M + H]⁺; ¹H NMR (400 MHz, DMSO-d₆) δ 10.17 (br s, 1H), 8.45 (s, 1H), 7.84 (d, J = 8.8 Hz, 2H), 7.75 (d, J = 8.8 Hz, 2H), 7.22 (s, 2H), 4.54-4.45 (m, 2H), 3.18- 3.16 (m, 1H), 2.84-2.83 (m, 1H), 1.83-1.69 (m, 3H), 1.53-1.46 (m, 1H), 1.26-1.22 (m, 1H), 0.93 (d, J = 6.6 Hz, 3H). |
| 72 | 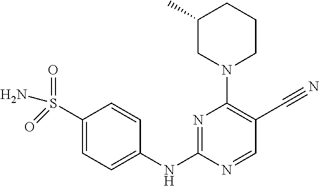 [SMILES: C[C@@H]1CCCN(c2nc(Nc3ccc(S(N)(=O)=O)cc3)ncc2C#N)C1] | LC-MS (ESI): m/z 373.1 [M + H]⁺; ¹H NMR (400 MHz, DMSO-d₆) δ 10.18 (s, 1H), 8.46 (s, 1H), 7.84 (d, J = 9.2 Hz, 2H), 7.75 (d, J = 9.2 Hz, 2H), 7.22 (s, 2H), 4.54-4.46 (m, 2H), 3.18-3.13 (m, 1H), 2.86-2.83 (m, 1H), 1.86- 1.73 (m, 3H), 1.54-1.47 (m, 1H), 1.27-1.23 (m, 1H), 0.93 (d, J = 6.6 Hz, 3H). |
| 73 | 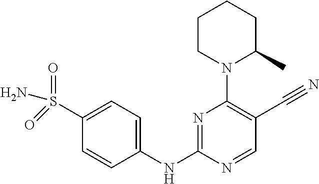 [SMILES: C[C@@H]1CCCCN1c1nc(Nc2ccc(S(N)(=O)=O)cc2)ncc1C#N] | LC-MS (ESI): m/z 373.1 [M + H]⁺; ¹H NMR (400 MHz, DMSO-d₆) δ 10.17 (br s, 1H), 8.46 (s, 1H), 7.85 (d, J = 9.2 Hz, 2H), 7.75 (d, J = 9.2 Hz, 2H), 7.22 (s, 2H), 4.94 (br s, 1H), 4.50-4.46 (m, 1H), 3.18-3.16 (m, 1H), 1.78- 1.51 (m, 6H), 1.32 (d, J = 6.8 Hz, 3H). |
| 74 | 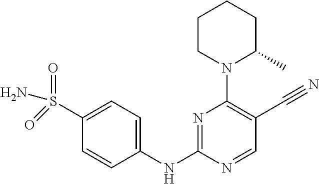 [SMILES: C[C@H]1CCCCN1c1nc(Nc2ccc(S(N)(=O)=O)cc2)ncc1C#N] | LC-MS (ESI): m/z 373.1 [M + H]⁺; ¹H NMR (400 MHz, DMSO-d₆) δ 10.15 (br s, 1H), 8.45 (s, 1H), 7.84 (d, J = 8.8 Hz, 2H), 7.75 (d, J = 8.8 Hz, 2H), 7.21 (s, 2H), 4.93 (br s, 1H), 4.49-4.45 (m, 1H), 3.21-3.18 (m, 1H), 1.77- 1.60 (m, 6H), 1.31 (d, J = 6.8 Hz, 3H). |
| 75 | 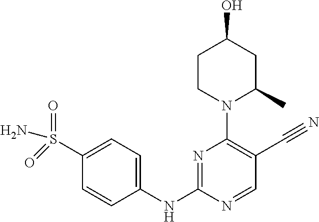 [SMILES: C[C@@H]1C[C@H](O)CCN1c1nc(Nc2ccc(S(N)(=O)=O)cc2)ncc1C#N] | LC-MS (ESI): m/z 389.1 [M + H]⁺; ¹H NMR (400 MHz, DMSO-d₆) δ 10.16 (br s, 1H), 8.46 (s, 1H), 7.86 (d, J = 8.8 Hz, 2H), 7.76 (d, J = 8.8 Hz, 2H), 7.21 (s, 2H), 4.85-4.80 (m, 2H), 4.37- 4.34 (m, 1H), 4.02-4.01 (m, 1H), 3.57-3.49 (m, 1H), 1.85-1.69 (m, 4H), 1.48 (d, J = 6.8 Hz, 3H). |
| 76 | 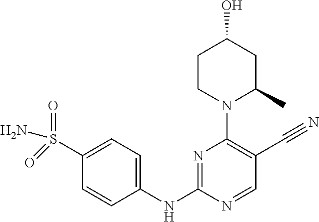 [SMILES: C[C@@H]1C[C@@H](O)CCN1c1nc(Nc2ccc(S(N)(=O)=O)cc2)ncc1C#N] | LC-MS (ESI): m/z 389.1 [M + H]⁺; ¹H NMR (400 MHz, DMSO-d₆) δ 10.18 (br s, 1H), 8.47 (s, 1H), 7.84 (d, J = 8.8 Hz, 2H), 7.76 (d, J = 8.8 Hz, 2H), 7.21 (s, 2H), 4.98 (br s, 1H), 4.79 (d, J = 4.8 Hz, 1H), 4.56-4.49 (m, 1H), 3.94-3.90 (m, 1H), 3.26-3.18 (m, 1H), 1.98-1.84 (m, 2H), 1.50-1.33 (m, 2H), 1.32 (d, J = 7.2 Hz, 3H). |
| 77 | 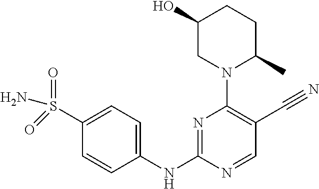 [SMILES: C[C@@H]1CC[C@H](O)CN1c1nc(Nc2ccc(S(N)(=O)=O)cc2)ncc1C#N] | LC-MS (ESI): m/z 389.1 [M + H]⁺; ¹H NMR (400 MHz, DMSO-d₆) δ 10.18 (br s, 1H), 8.47 (s, 1H), 7.86 (d, J = 8.8 Hz, 2H), 7.76 (d, J = 8.8 Hz, 2H), 7.22 (s, 2H), 5.17 (d, J = 5.2 Hz, 1H), 4.85 (br s, 1H), 4.57-4.54 (m, 1H), 3.53-3.49 (m, 1H), 2.89-2.82 (m, 1H), 1.73-1.59 (m, 4H), 1.29 (d, J = 6.8 Hz, 3H). |
| 78 | 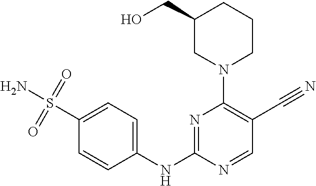 [SMILES: N#Cc1cnc(Nc2ccc(S(N)(=O)=O)cc2)nc1N1CCC[C@H](CO)C1] | LC-MS (ESI): m/z 389.1 [M + H]⁺; ¹H NMR (400 MHz, DMSO-d₆) δ 10.18 (br s, 1H), 8.46 (s, 1H), 7.87 (d, J = 8.8 Hz, 2H), 7.75 (d, J = 8.8 Hz, 2H), 7.20 (s, 2H), 4.69-4.65 (m, 2H), 4.53- 4.49 (m, 1H), 3.40-3.36 (m, 1H), 3.23-3.15 (m, 1H), 2.98-2.92 (m, 1H), 1.79-1.72 (m, 3H), 1.54-1.51 (m, 1H), 1.29-1.22 (m, 2H). |
| 79 | 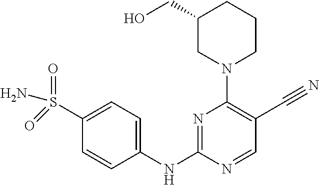 [SMILES: N#Cc1cnc(Nc2ccc(S(N)(=O)=O)cc2)nc1N1CCC[C@@H](CO)C1] | LC-MS (ESI): m/z 389.1 [M + H]⁺; ¹H NMR (400 MHz, DMSO-d₆) δ 10.18 (br s, 1H), 8.46 (s, 1H), 7.87 (d, J = 8.8 Hz, 2H), 7.75 (d, J = 8.8 Hz, 2H), 7.20 (s, 2H), 4.69-4.64 (m, 2H), 4.54- 4.48 (m, 1H), 3.40-3.36 (m, 1H), 3.29-3.20 (m, 1H), 2.98-2.92 (m, 1H), 1.80-1.72 (m, 3H), 1.56-1.51 (m, 1H), 1.361-1.31 (m, 1H). |
| 80 | 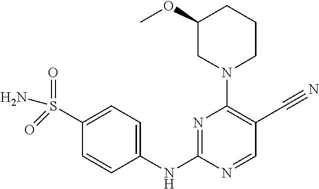 [SMILES: CO[C@H]1CCCN(c2nc(Nc3ccc(S(N)(=O)=O)cc3)ncc2C#N)C1] | LC-MS (ESI): m/z 389.1 [M + H]⁺; ¹H NMR (400 MHz, DMSO-d₆) δ 10.18 (br s, 1H), 8.47 (s, 1H), 7.84 (d, J = 8.8 Hz, 2H), 7.75 (d, J = 8.8 Hz, 2H), 7.22 (s, 2H), 4.15-4.11 (m, 1H), 3.92- 3.87 (m, 1H), 3.78-3.72 (m, 2H), 3.27 (s, 4H), 1.96-1.79 (m, 2H), 1.67-1.51 (m, 2H). |
| 81 | 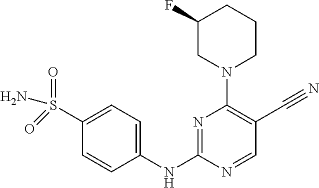 [SMILES: N#Cc1cnc(Nc2ccc(S(N)(=O)=O)cc2)nc1N1CCC[C@H](F)C1] | LC-MS (ESI): m/z 377.1 [M + H]⁺; ¹H NMR (400 MHz, DMSO-d₆) δ 10.23 (br s, 1H), 8.50 (s, 1H), 7.84 (d, J = 8.8 Hz, 2H), 7.76 (d, J = 8.8 Hz, 2H), 7.22 (s, 2H), 4.91 (d, J_{HF} = 47.5 Hz, 1H), 4.43-4.22 (m, 2H), 3.86-3.75 (m, 1H), 3.56-3.33 (m, 1H), 1.96- 1.63 (m, 4H). |
| 82 | 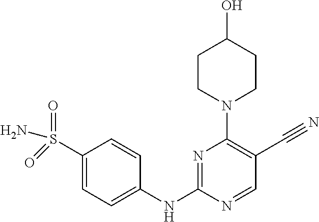 [SMILES: N#Cc1cnc(Nc2ccc(S(N)(=O)=O)cc2)nc1N1CCC(O)CC1] | LC-MS (ESI): m/z 375.1 [M + H]⁺; ¹H NMR (400 MHz, DMSO-d₆) δ 10.18 (br s, 1H), 8.48 (s, 1H), 7.84 (d, J = 8.8 Hz, 2H), 7.76 (d, J = 8.8 Hz, 2H), 7.21 (s, 2H), 4.84 (d, J = 4.2 Hz, 1H), 4.23-4.18 (m, 2H), 3.82-3.80 (m, 1H), 3.57-3.51 (m, 2H), 1.89-1.85 (m, 2H), 1.50-1.42 (m, 2H). |
| 83 | 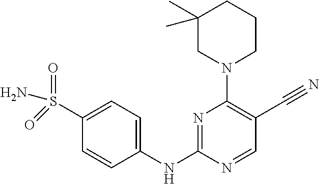 [SMILES: CC1(C)CCCN(c2nc(Nc3ccc(S(N)(=O)=O)cc3)ncc2C#N)C1] | LC-MS (ESI): m/z 387.2 [M + H]⁺; ¹H NMR (400 MHz, DMSO-d₆) δ 10.16 (br s, 1H), 8.45 (s, 1H), 7.84 (d, J = 8.8 Hz, 2H), 7.75 (d, J = 8.8 Hz, 2H), 7.22 (s, 2H), 3.84-3.81 (m, 2H), 3.65 (s, 2H), 1.69-1.62 (m, 2H), 1.48- 1.46 (m, 2H), 0.93 (s, 6H). |
| 84 | 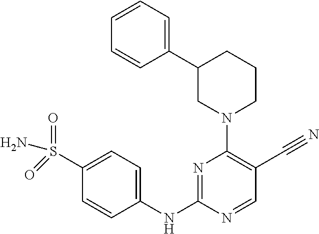 [SMILES: N#Cc1cnc(Nc2ccc(S(N)(=O)=O)cc2)nc1N1CCCC(c2ccccc2)C1] Racemic mixture | LC-MS (ESI): m/z 435.2 [M + H]⁺; ¹H NMR (400 MHz, DMSO-d₆) δ 10.20 (br s, 1H), 8.50 (s, 1H), 7.84 (d, J = 8.8 Hz, 2H), 7.69 (d, J = 8.8 Hz, 2H), 7.40-7.34 (m, 4H), 7.30-7.26 (m, 1H), 7.22 (s, 2H), 4.77-4.73 (m, 2H), 3.22-3.18 (m, 2H), 2.90-2.83 (m, 1H), 2.04-1.87 (m, 3H), 1.69- 1.65 (m, 1H). |
| 85 | 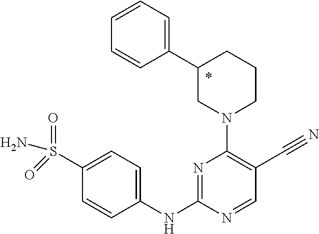 [SMILES: N#Cc1cnc(Nc2ccc(S(N)(=O)=O)cc2)nc1N1CCCC(c2ccccc2)C1] Single isomer | LC-MS (ESI): m/z 435.2 [M + H]⁺; ¹H NMR (400 MHz, DMSO-d₆) δ 10.20 (br s, 1H), 8.50 (s, 1H), 7.83 (d, J = 8.8 Hz, 2H), 7.69 (d, J = 8.8 Hz, 2H), 7.39-7.33 (m, 4H), 7.29-7.25 (m, 1H), 7.20 (s, 2H), 4.76-4.73 (m, 2H), 3.24-3.13 (m, 2H), 2.91-2.82 (m, 1H), 2.01-1.85 (m, 3H), 1.71- 1.63 (m, 1H). |
| 86 | 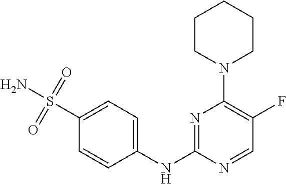 [SMILES: NS(=O)(=O)c1ccc(Nc2ncc(F)c(N3CCCCC3)n2)cc1] | LC-MS (ESI): m/z 352.1 [M + H]⁺; ¹H NMR (400 MHz, DMSO-d₆) δ 9.56 (s, 1H), 8.03 (d, J_{HF} = 7.0 Hz, 1H), 7.83 (d, J = 8.8 Hz, 2H), 7.68 (d, J = 8.8 Hz, 2H), 7.12 (s, 2H), 3.71-3.69 (m, 4H), 1.65-1.60 (m, 6H). |
| 87 | 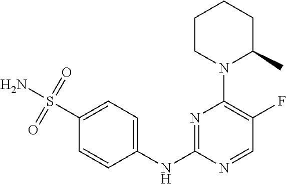 [SMILES: C[C@@H]1CCCCN1c1nc(Nc2ccc(S(N)(=O)=O)cc2)ncc1F] | LC-MS (ESI): m/z 366.1 [M + H]⁺; ¹H NMR (400 MHz, DMSO-d₆) δ 9.55 (s, 1H), 8.02 (d, J_{HF} = 7.2 Hz, 1H), 7.84 (d, J = 8.8 Hz, 2H), 7.69 (d, J = 8.8 Hz, 2H), 7.12 (s, 2H), 4.72 (br s, 1H), 4.23- 4.20 (m, 1H), 3.17-3.10 (m, 1H), 1.74-1.70 (m, 3H), 1.59-1.48 (m, 3H), 1.27 (d, J = 6.8 Hz, 3H). |
| 88 | 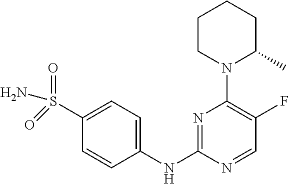 [SMILES: C[C@H]1CCCCN1c1nc(Nc2ccc(S(N)(=O)=O)cc2)ncc1F] | LC-MS (ESI): m/z 366.1 [M + H]⁺; ¹H NMR (400 MHz, DMSO-d₆) δ 9.54 (br s, 1H), 8.02 (d, J_{HF} = 7.2 Hz, 1H), 7.83 (d, J = 8.8 Hz, 2H), 7.68 (d, J = 8.8 Hz, 2H), 7.12 (s, 2H), 4.72 (br s, 1H), 4.23-4.20 (m, 1H), 3.17-3.11 (m, 1H), 1.71-1.50 (m, 6H), 1.27 (d, J = 6.8 Hz, 3H). |
| 89 | 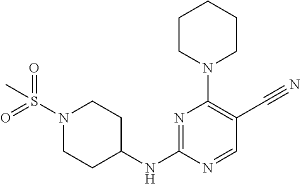 [SMILES: CS(=O)(=O)N1CCC(Nc2ncc(C#N)c(N3CCCCC3)n2)CC1] | LC-MS (ESI): m/z 365.2 [M + H]⁺; ¹H NMR (400 MHz, DMSO-d₆) δ 8.29 & 8.21 (s, 1H), 7.72 & 7.53 (d, J = 7.8 Hz, 1H), 3.90-3.75 (m, 5H), 3.53-3.50 (m, 2H), 2.89-2.82 (m, 2H), 2.86 (s, 3H), 1.95-1.87 (m, 2H), 1.64-1.50 (m, 8H). |
| 90 | 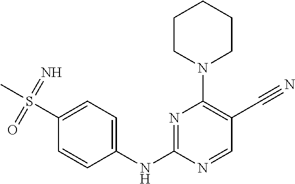 [SMILES: CS(=N)(=O)c1ccc(Nc2ncc(C#N)c(N3CCCCC3)n2)cc1] | LC-MS (ESI): m/z 357.1 [M + H]⁺; ¹H NMR (400 MHz, DMSO-d₆) δ 10.21 (br s, 1H), 8.47 (s, 1H), 7.90-7.81 (m, 4H), 4.05 (s, 1H), 3.89-3.82 (m, 4H), 3.03 (s, 3H), 1.72-1.60 (m, 6H). |
| 91 | 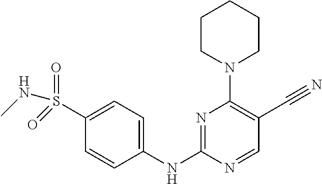 [SMILES: CNS(=O)(=O)c1ccc(Nc2ncc(C#N)c(N3CCCCC3)n2)cc1] | LC-MS (ESI): m/z 373.0 [M + H]⁺; ¹H NMR (400 MHz, DMSO-d₆) δ 10.21 (br s, 1H), 8.47 (s, 1H), 7.89 (d, J = 8.8 Hz, 2H), 7.71 (d, J = 8.8 Hz, 2H), 7.27 (br, 1H), 3.94-3.81 (m, 4H), 2.39 (s, 3H), 1.75-1.56 (m, 6H). |
| 93 | 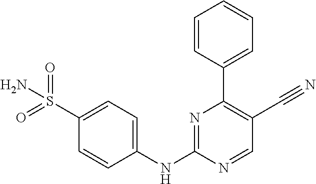 [SMILES: N#Cc1cnc(Nc2ccc(S(N)(=O)=O)cc2)nc1-c1ccccc1] | LC-MS (ESI): m/z 352.1 [M + H]⁺; ¹H NMR (400 MHz, DMSO-d₆) δ 10.86 (br s, 1H), 9.04 (s, 1H), 8.00-7.94 (m, 4H), 7.81 (d, J = 8.8 Hz, 2H), 7.66-7.61 (m, 3H), 7.26 (s, 2H). |
| 94 | 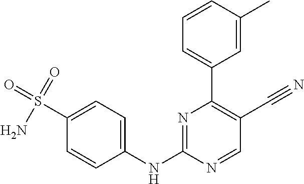 [SMILES: Cc1cccc(-c2nc(Nc3ccc(S(N)(=O)=O)cc3)ncc2C#N)c1] | LC-MS (ESI): m/z 366.1 [M + H]⁺; ¹H NMR (400 MHz, DMSO-d₆) δ 10.85 (br s, 1H), 9.02 (s, 1H), 7.96 (d, J = 8.8 Hz, 2H), 7.81-7.78 (m, 4H), 7.53-7.46 (m, 2H), 7.26 (s, 2H), 2.43 (s, 3H). |
| 95 | 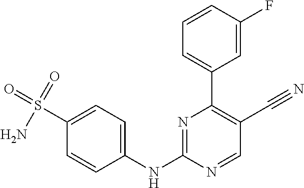 [SMILES: N#Cc1cnc(Nc2ccc(S(N)(=O)=O)cc2)nc1-c1cccc(F)c1] | LC-MS (ESI): m/z 370.1 [M + H]⁺; ¹H NMR (400 MHz, DMSO-d₆) δ 10.89 (br s, 1H), 9.06 (s, 1H), 7.95 (d, J = 8.8 Hz, 2H), 7.85-7.76 (m, 4H), 7.71-7.66 (m, 1H), 7.53-7.49 (m, 1H), 7.27 (s, 2H). |
| 96 | 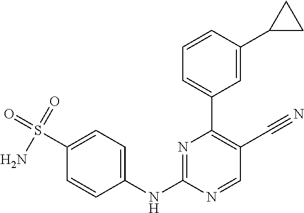 [SMILES: N#Cc1cnc(Nc2ccc(S(N)(=O)=O)cc2)nc1-c1cccc(C2CC2)c1] | LC-MS (ESI): m/z 392.1 [M + H]⁺; ¹H NMR (400 MHz, DMSO-d₆) δ 10.85 (s, 1H), 9.03 (s, 1H), 7.97 (d, J = 8.8 Hz, 2H), 7.81 (d, J = 8.8 Hz, 2H), 7.76 (d, J = 7.6 Hz, 1H), 7.65 (s, 1H), 7.49 (t, J = 7.6 Hz, 1H), 7.40 (d, J = 7.6 Hz, 1H), 7.26 (s, 2H), 1.19-1.15 (m, 1H), 1.05-1.03 (m, 2H), 0.79- 0.75 (m, 2H). |
| 97 | 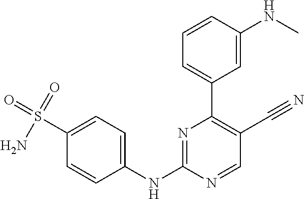 [SMILES: CNc1cccc(-c2nc(Nc3ccc(S(N)(=O)=O)cc3)ncc2C#N)c1] | LC-MS (ESI): m/z 381.1 [M + H]⁺; ¹H NMR (400 MHz, DMSO-d₆) δ 10.81 (s, 1H), 9.00 (s, 1H), 7.98 (d, J = 8.8 Hz, 2H), 7.80 (d, J = 8.8 Hz, 2H), 7.30 (t, J = 7.8 Hz, 1H), 7.26 (s, 2H), 7.16 (d, J = 7.4 Hz, 1H), 7.11 (s, 1H), 6.80-6.78 (m, 1H), 6.06 (d, J = 5.2 Hz, 1H), 2.75 (d, J = 5.2 Hz, 3H). |
| 98 | 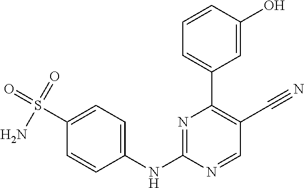 [SMILES: N#Cc1cnc(Nc2ccc(S(N)(=O)=O)cc2)nc1-c1cccc(O)c1] | LC-MS (ESI): m/z 368.1 [M + H]⁺; ¹H NMR (400 MHz, DMSO-d₆) δ 10.84 (br s, 1H), 9.93 (br s, 1H), 9.02 (s, 1H), 7.97 (d, J = 8.8 Hz, 2H), 7.81 (d, J = 8.8 Hz, 2H), 7.42-7.39 (m, 3H), 7.26 (s, 2H), 7.04-7.01 (m, 1H). |
| 105a | 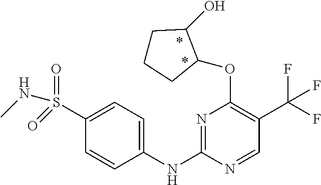 [SMILES: CNS(=O)(=O)c1ccc(Nc2ncc(C(F)(F)F)c(OC3CCCC3O)n2)cc1] Enantiomer 1 (earlier eluting enantiomer), from cis-cyclopentane 1,2-diol | LC-MS (ESI): m/z 433.2 [M + H]⁺; ¹H NMR (400 MHz, DMSO-d₆) δ 10.45 (s, 1H), 8.57 (s, 1H), 7.95 (d, J = 8.8 Hz, 2H), 7.72 (d, J = 8.8 Hz, 2H), 7.31 (q, J = 9.6 Hz, 1H), 5.50-5.23 (m, 1H), 4.71 (d, J = 4.8 Hz, 1H), 4.39-4.09 (m, 1H), 2.40 (d, J = 4.8 Hz, 3H), 2.05-1.90 (m, 1H), 1.81- 1.75 (m, 3H), 1.68-1.45 (m, 2H). ee: 78.7% Retention time: 3.02 min; Column: ChiralPak AD, 250 × 4.6 mm I.D., 5 μm, Mobile phase: A for CO₂ and B for methanol (0.05% DEA), Gradient 10 min |
| | | @ 50%, Flow rate: 1.8 mL/min, |
| | | Back pressure: 100 bar, Column |
| | | temperature: 35° C. |
| 105b | 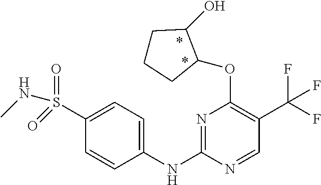 [SMILES: CNS(=O)(=O)c1ccc(Nc2ncc(C(F)(F)F)c(OC3CCCC3O)n2)cc1] Enantiomer 2 (later eluting enantiomer), from cis-cyclopentane 1,2-diol | LC-MS (ESI): m/z 433.2 [M + H]⁺. ee: 82.9% Retention time: 3.91 min; Column: ChiralPak AD, 250 × 4.6 mm I.D., 5 μm, Mobile phase: A for CO₂ and B for methanol (0.05% DEA), Gradient, 10 min @ 50%, Flow rate: 1.8 mL/min, Back pressure: 100 bar, Column temperature: 35° C. |
| 106a | 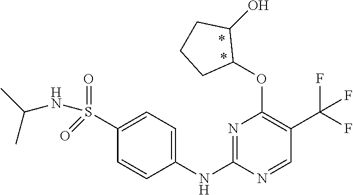 [SMILES: CC(C)NS(=O)(=O)c1ccc(Nc2ncc(C(F)(F)F)c(OC3CCCC3O)n2)cc1] Enantiomer 1 (earlier eluting enantiomer), from cis-cyclopentane 1,2-diol | LC-MS (ESI): m/z 461.2 [M + H]⁺; ¹H NMR (400 MHz, DMSO-d₆) δ 10.43 (s, 1H), 8.57 (s, 1H), 7.92 (d, J = 8.9 Hz, 2H), 7.79-7.71 (m, 2H), 7.42 (d, J = 7.2 Hz, 1H), 5.39-5.34 (m, 1H), 4.69 (d, J = 4.7 Hz, 1H), 4.27-4.19 (m, 1H), 3.22-3.17 (m, 1H), 2.05-1.92 (m, 1H), 1.87-1.74 (m, 3H), 1.68- 1.50 (m, 2H), 0.94 (d, J = 6.5 Hz, 6H). ee: 99.1% Retention time: 2.66 min; Column: ChiralPak IC, 250 × 4.6 mm I.D., 5 μm, Mobile phase: A for CO₂ and B for MeOH (0.05% |
| | | DEA), Gradient: 8 min @ B 40%, |
| | | Flow rate: 1.8 mL/min, Back |
| | | pressure: 100 bar, Column |
| | | temperature: 35° C. |
| 106b | 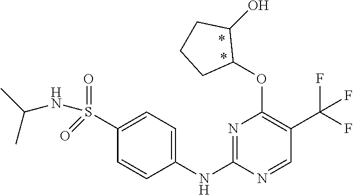 [SMILES: CC(C)NS(=O)(=O)c1ccc(Nc2ncc(C(F)(F)F)c(OC3CCCC3O)n2)cc1] Enantiomer 2 (later eluting enantiomer), from cis-cyclopentane 1,2-diol | LC-MS (ESI): m/z 461.2 [M + H]⁺. ee: 97.3% Retention time: 3.21 min; Column: ChiralPak IC, 250 × 4.6 mm I.D., 5 μm, Mobile phase: A for CO₂ and B for MeOH (0.05% DEA), Gradient: 8 min @ B 40%, Flow rate: 1.8 mL/min, Back pressure: 100 bar, Column temperature: 35° C. |
| 107a | 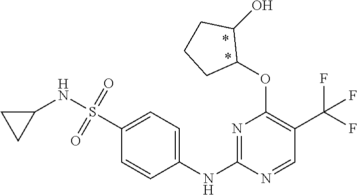 [SMILES: O=S(=O)(NC1CC1)c1ccc(Nc2ncc(C(F)(F)F)c(OC3CCCC3O)n2)cc1] Enantiomer 1 (earlier eluting enantiomer), from cis-cyclopentane 1,2-diol | LC-MS (ESI): m/z 459.2 [M + H]⁺; ¹H NMR (400 MHz, DMSO-d₆) δ 10.45 (s, 1H), 8.57 (s, 1H), 7.94 (d, J = 8.7 Hz, 2H), 7.75 (d, J = 8.7 Hz, 2H), 5.40-5.35 (m, 1H), 4.71 (s, 1H), 4.27-4.21 (m, 1H), 2.17-1.92 (m, 2H), 1.91- 1.53 (m, 5H), 0.50-0.33 (m, 4H). ee: 99.3% Retention time: 3.77 min; Column: ChiralPak IA, 250 × 4.6 mm I.D. 5 μm, Mobile phase: A for CO₂ and B for MeOH (0.05% DEA), Gradient: 8 min @ B 40%, Flow rate: 1.8 mL/min, Back pressure: 100 bar, Column |
| | | temperature: 35° C. |
| 107b | 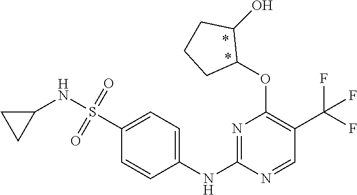 [SMILES: O=S(=O)(NC1CC1)c1ccc(Nc2ncc(C(F)(F)F)c(OC3CCCC3O)n2)cc1] Enantiomer 2 (later eluting enantiomer), from cis-cyclopentane 1,2-diol | LC-MS (ESI): m/z 459.2 [M + H]⁺. ee: 95% Retention time: 4.91 min; Column: ChiralPak IA, 250 × 4.6 mm I.D., 5 μm, Mobile phase: A for CO₂ and B for MeOH (0.05% DEA), Gradient: 8 min @ B 40%, Flow rate: 1.8 mL/min, Back pressure: 100 bar, Column temperature: 35° C. |
| 108 | 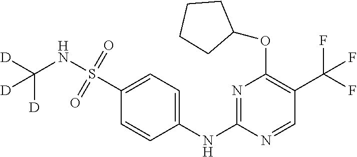 [SMILES: [2H]C([2H])([2H])NS(=O)(=O)c1ccc(Nc2ncc(C(F)(F)F)c(OC3CCCC3)n2)cc1] | LC-MS (ESI): m/z 420.2 [M + H]⁺; ¹H NMR (400 MHz, DMSO-d₆) δ 10.51 (s, 1H), 8.57 (s, 1H), 7.96 (d, J = 8.0 Hz, 2H), 7.73 (d, J = 8.0 Hz, 2H), 7.25 (s, 1H), 5.67-4.53 (m, 1H), 2.06-1.92 (m, 2H), 1.86-1.73 (m, 2H), 1.76- 1.60 (m, 4H). |
| 109a | 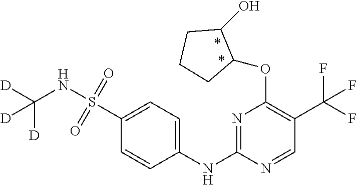 [SMILES: [2H]C([2H])([2H])NS(=O)(=O)c1ccc(Nc2ncc(C(F)(F)F)c(OC3CCCC3O)n2)cc1] Enantiomer 1 (earlier eluting enantiomer), from cis-cyclopentane 1,2-diol | LC-MS (ESI): m/z 436.2 [M + H]⁺; ¹H NMR (400 MHz, DMSO-d₆) δ 10.45 (s, 1H), 8.58 (s, 1H), 7.95 (d, J = 8.0 Hz, 2H), 7.73 (d, J = 8.0 Hz, 2H), 7.26 (s, 1H), 5.41-5.37 (m, 1H), 4.71 (d, J = 4.0 Hz, 1H), 4.32-4.20 (m, 1H), 2.10-1.90 (m, 1H), 1.89-1.79 (m, 3H), 1.67-1.55 (m, 2H). ee: 96.8% Retention time: 3.47 min; Column: ChiralPak AD, 250 × 4.6 mm I.D., 5 μm, Mobile phase: A for CO₂ and B for methanol (0.05% DEA), Gradient: 10 min |
| | | @ 40%, Flow rate: 2.0 mL/min, |
| | | Back pressure: 100 bar, Column |
| | | temperature: 35° C. |
| 109b | 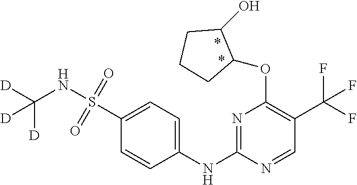 [SMILES: [2H]C([2H])([2H])NS(=O)(=O)c1ccc(Nc2ncc(C(F)(F)F)c(OC3CCCC3O)n2)cc1] Enantiomer 2 (later eluting enantiomer), from cis-cyclopentane 1,2-diol | LC-MS (ESI): m/z 436.0 [M + H]⁺. ee: 95.2% Retention time: 4.87 min; Column: ChiralPak AD, 250 × 4.6 mm I.D., 5 μm, Mobile phase: A for CO₂ and B for methanol (0.05% DEA), Gradient: 10 min @ 40%, Flow rate: 2.0 mL/min, Back pressure: 100 bar, Column temperature: 35° C. |
| 110a | 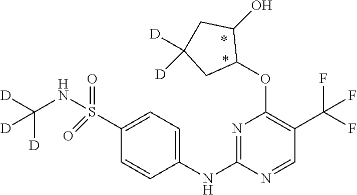 [SMILES: [2H]C1([2H])CC(O)C(Oc2nc(Nc3ccc(S(=O)(=O)NC([2H])([2H])[2H])cc3)ncc2C(F)(F)F)C1] Enantiomer 1 (earlier eluting enantiomer), from cis- cyclopentane-4,4-d₂-1,2-diol | LC-MS (ESI): m/z 438.2 [M + H]⁺; ¹H NMR (400 MHz, DMSO-d₆) δ 10.45 (s, 1H), 8.57 (s, 1H), 7.95 (d, J = 8.8 Hz, 2H), 7.73 (d, J = 8.8 Hz, 2H), 7.27 (s, 1H), 5.41-5.37 (m, 1H), 4.72 (d, J = 4.6 Hz, 1H), 4.28-4.21 (m, 1H), 2.03-1.98 (m, 1H), 1.92-1.72 (m, 2H), 1.79-1.60 (m, 1H). ee: 100% Retention time: 1.49 min; Column: Chiralpak AD-3, 150 × 4.6 mm I.D., 3 μm, Mobile phase: 40% of ethanol (0.05% DEA) in CO₂, Flow rate: 2.5 mL/min, Column temperature: 35° C. |
| 110b | 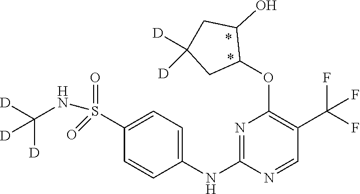 [SMILES: [2H]C1([2H])CC(O)C(Oc2nc(Nc3ccc(S(=O)(=O)NC([2H])([2H])[2H])cc3)ncc2C(F)(F)F)C1] Enantiomer 2 (later eluting enantiomer), from cis- cyclopentane-4,4-d₂-1,2-diol | LC-MS (ESI): m/z 438.2 [M + H]⁺. ee: 99.5% Retention time: 2.04 min; Column: Chiralpak AD-3, 150 × 4.6 mm I.D., 3 μm, Mobile phase: 40% of ethanol (0.05% DEA) in CO₂, Flow rate: 2.5 mL/min, Column temperature: 35° C. |
| 111a | 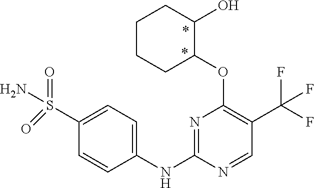 [SMILES: NS(=O)(=O)c1ccc(Nc2ncc(C(F)(F)F)c(OC3CCCCC3O)n2)cc1] Enantiomer 1 (earlier eluting enantiomer), made from cis- cyclohexane-1,2-diol | LC-MS (ESI): m/z 433.1 [M + H]⁺; ¹H NMR (400 MHz, DMSO-d₆) δ 10.42 (s, 1H), 8.57 (s, 1H), 7.88 (d, J = 8.8 Hz, 2H), 7.76 (d, J = 8.9 Hz, 2H), 7.23 (s, 2H), 5.44 (s, 1H), 4.73 (d, J = 4.3 Hz, 1H), 3.84 (s, 1H), 2.0-1.94 (m, 1H), 1.74-1.55 (m, 4H), 1.49- 1.30 (m, 3H). ee: 99.3% Retention time: 3.14 min; Column: ChiralPak AD, 250 × 4.6 mm I.D., 5 μm, Mobile phase: A for CO₂ and B for MeOH (0.05% DEA), Gradient: 8 min @ B 40%, Flow rate: 2.0 mL/min, Back |
| | | pressure: 100 bar, Column |
| | | temperature: 35° C. |
| 111b | 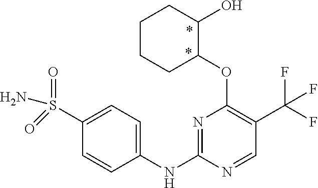 [SMILES: NS(=O)(=O)c1ccc(Nc2ncc(C(F)(F)F)c(OC3CCCCC3O)n2)cc1] Enantiomer 2 (later eluting enantiomer), made from cis- cyclohexane-1,2-diol | LC-MS (ESI): m/z 433.1 [M + H]⁺; ¹H NMR (400 MHz, DMSO-d₆) δ 10.42 (br s, 1H), 8.58 (s, 1H), 7.89 (d, J = 8.8 Hz, 2H), 7.77 (d, J = 8.8 Hz, 2H), 7.24 (s, 2H), 5.45 (s, 1H), 4.73 (d, J = 4.4 Hz, 1H), 3.84 (s, 1H), 2.02- 1.92 (m, 1H), 1.72-1.60 (s, 4H), 1.52-1.30 (m, 3H). ee: 97.6% ee. Retention time: 3.99 min. Column: ChiralPak AD, 250 × 4.6 mm I.D., 5 μm, Mobile phase: A for CO₂ and B for methanol (0.05% DEA), Gradient: 10 min @ 40%. Flow rate: 2.0 mL/min, |
| | | Back pressure: 100 bar, Column |
| | | temperature: 35° C. |
| 112a | 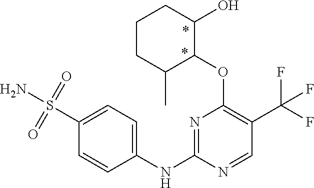 [SMILES: CC1CCCC(O)C1Oc1nc(Nc2ccc(S(N)(=O)=O)cc2)ncc1C(F)(F)F] Enantiomer 1 (earlier eluting enantiomer), made from cis-3- methylcyclohexane-1,2-diol | LC-MS (ESI): m/z 447.2 [M + H]⁺; ¹H NMR (400 MHz, DMSO-d₆) δ 10.43 (s, 1H), 8.58 (s, 1H), 7.87 (d, J = 8.8 Hz, 2H), 7.76 (d, J = 8.8 Hz, 2H), 7.25 (s, 2H), 4.81 (d, J = 7.6 Hz, 1H), 4.63 (d, J = 4.8 Hz, 1H), 4.26 (s, 1H), 2.25-2.12 (m, 1H), 1.83-1.38 (m, 6H), 0.93 (d, J = 6.7 Hz, 3H). ee: 100% Retention time: 2.35 min; Column: ChiralPak AD, 250 × 4.6 mm I.D., 5 μm, Mobile phase: A for CO₂ and B for methanol (0.05% DEA), Gradient: 10 min @ 40%, Flow rate: 2.0 mL/min, |
| | | Back pressure: 100 bar, Column |
| | | temperature: 35° C. |
| 112b | 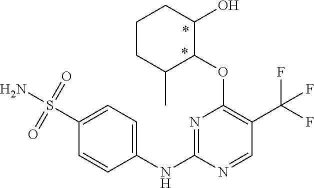 [SMILES: CC1CCCC(O)C1Oc1nc(Nc2ccc(S(N)(=O)=O)cc2)ncc1C(F)(F)F] Enantiomer 2 (later eluting enantiomer), made from cis-3- methylcyclohexane-1,2-diol | LC-MS (ESI): m/z: 447.2 [M + H]⁺. ee: 90.5% Retention time: 2.86 min; Column: ChiralPak AD, 250 × 4.6 mm I.D., 5 μm, Mobile phase: A for CO₂ and B for methanol (0.05% DEA), Gradient: 10 min @ 40%, Flow rate: 2.0 mL/min, Back pressure: 100 bar, Column temperature: 35° C. |
| 113a | 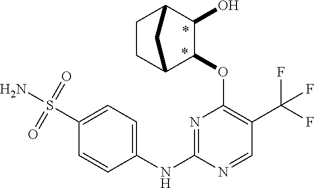 [SMILES: NS(=O)(=O)c1ccc(Nc2ncc(C(F)(F)F)c(O[C@H]3[C@@H]4CC[C@@H](C4)[C@H]3O)n2)cc1] Enantiomer 1 (earlier eluting enantioimer), made from cis- bicyclo[2.2.1]heptane-2,3-diol | LC-MS (ESI): m/z 445.1 [M + H]⁺. ee: 43.0% Retention time: 3.25 min; Column: ChiralPak AD, 250 × 4.6 mm I.D., 5 μm, Mobile phase: A for CO₂ and B for methanol (0.05% DEA), Gradient: 10 min @ 40%, Flow rate: 2.0 mL/min, Back pressure: 100 bar, Column temperature: 35° C. |
| 113b | 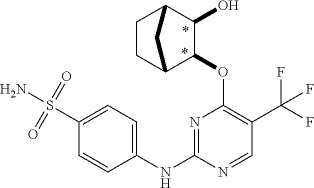 [SMILES: NS(=O)(=O)c1ccc(Nc2ncc(C(F)(F)F)c(O[C@H]3[C@@H]4CC[C@@H](C4)[C@H]3O)n2)cc1] Enantiomer 2 (later eluting enantiomer), made from cis- bicyclo[2.2.1]heptane-2,3-diol | LC-MS (EIS): m/z 445.1 [M + H]⁺; ¹H NMR (400 MHz, DMSO-d₆) δ 10.36 (s, 1H), 8.53 (s, 1H), 7.91 (d, J = 8.8 Hz, 2H), 7.76 (d, J = 8.8 Hz, 2H), 7.23 (s, 2H), 4.91 (d, J = 5.3 Hz, 1H), 4.63 (d, J = 5.0 Hz, 1H), 3.86 (t, J = 5.0 Hz, 1H), 2.33-2.85 (m, 1H), 2.10- 2.02 (m, 1H), 1.92 (d, J = 9.6 Hz, 1H), 1.53-1.48 (m, 2H), 1.24-1.19 (m, 1H), 1.15-1.10 (m, 2H). ee: 85.8% Retention time: 4.22 min; Column: ChiralPak AD, 250 × 4.6 mm I.D., 5 μm, Mobile phase: A for CO₂ and B for methanol |
| | | (0.05% DEA), Gradient: 10 min |
| | | @ 40%, Flow rate: 2.0 mL/min, |
| | | Back pressure: 100 bar, Column |
| | | temperature: 35° C. |
| 114a | 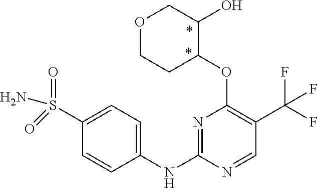 [SMILES: NS(=O)(=O)c1ccc(Nc2ncc(C(F)(F)F)c(OC3CCOCC3O)n2)cc1] Enantiomer 1 (earlier eluting enantiomer), made from cis- tetrahydro-2H-pyran-3,4-diol | LC-MS (ESI): m/z 435.2 [M + H]⁺; ¹H NMR (400 MHz, DMSO-d₆) δ 10.45 (s, 1H), 8.59 (s, 1H), 7.87 (d, J = 8.8 Hz, 2H), 7.77 (d, J = 8.8 Hz, 2H), 7.23 (s, 2H), 5.60-5.49 (m, 1H), 5.06 (d, J = 4.8 Hz, 1H), 3.93-3.86 (m, 1H), 3.68-3.52 (m, 4H), 2.10-1.98 (m, 1H), 1.92-1.78 (m, 1H). ee: 100% Retention time: 4.20 min; Column: ChiralPak IB, 100 × 4.6 mm I.D., 5 μm; Mobile phase: A for CO₂ and B for methanol (0.05% DEA); Gradient: 0.0 min- 1.0 min @ 10% @ B, 1.0 min-4.5 |
| | | min gradient (10-40% B), 4.5 |
| | | min-7.0 min @ 40% B, 7.0 min- |
| | | 8.0 min @ 10% B; Flow rate: 2.5 |
| | | mL/min; Column temperature: |
| | | 40° C. |
| 114b | 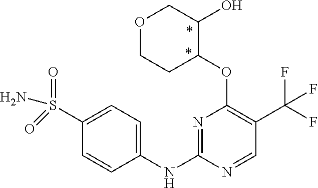 [SMILES: NS(=O)(=O)c1ccc(Nc2ncc(C(F)(F)F)c(OC3CCOCC3O)n2)cc1] Enantiomer 2 (later eluting enantiomer), made from cis- tetrahydro-2H-pyran-3,4-diol | LC-MS (ESI): m/z 435.2 [M + H]⁺. ee: 100% Retentioin time: 4.83 min; Column: ChiralPak IB, 100 × 4.6 mm I.D., 5 μm; Mobile phase: A for CO₂ and B for methanol (0.05% DEA); Gradient: 0.0 min- 1.0 min @ 10% B, 1.0 min-4.5 min gradient (10-40% B), 4.5 min-7.0 min @ 40% B, 7.0 min- 8.0 min @ 10% B; Flow rate: 2.5 mL/min; Column temperature: 40° C. |
| 115a | 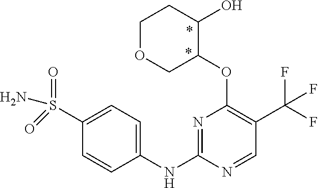 [SMILES: NS(=O)(=O)c1ccc(Nc2ncc(C(F)(F)F)c(OC3COCCC3O)n2)cc1] Enantiomer 1 (earlier eluting enantiomer), made from cis- tetrahydro-2H-pyran-3,4-diol | LC-MS (ESI): m/z 435.2 [M + H]⁺; ¹H NMR (400 MHz, DMSO-d₆) δ 10.42 (s, 1H), 8.59 (s, 1H), 7.86 (d, J = 8.8 Hz, 2H), 7.77 (d, J = 8.8 Hz, 2H), 7.23 (s, 2H), 5.40-5.34 (m, 1H), 5.01 (d, J = 4.0 Hz, 1H), 4.07-3.91 (m, 2H), 3.86-3.79 (m, 1H), 3.65-3.58 (m, 1H), 3.50-3.42 (m, 1H), 1.90-1.81 (m, 1H), 1.70-1.63 (m, 1H). ee: 100% Retention time: 4.05 min; Column: ChiralPak IB, 100 × 4.6 mm I.D., 5 μm; Mobile phase: A for CO₂ and B for methanol (0.05% DEA); Gradient: 0.0 min- |
| | | 1.0 min @ 10% B, 1.0 min-4.5 |
| | | min gradient (10-40% B), 4.5 |
| | | min-7.0 min @ 40% B, 7.0 min- |
| | | 8.0 min @ 10% B; Flow rate: 2.5 |
| | | mL/min; Column temperature: |
| | | 40° C. |
| 115b | 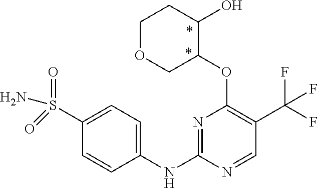 [SMILES: NS(=O)(=O)c1ccc(Nc2ncc(C(F)(F)F)c(OC3COCCC3O)n2)cc1] Enantiomer 2 (later eluting enantiomer), made from cis- tetrahydro-2H-pyran-3,4-diol | LC-MS (ESI): m/z 435.2 [M + H]⁺. ee: 100% Retention time: 4.47 min; Column: ChiralPak IB, 100 × 4.6 mm I.D., 5 μm; Mobile phase: A for CO₂ and B for methanol (0.05% DEA); Gradient: 0.0 min- 1.0 min @ 10% B, 1.0 min-4.5 min gradient (10-40% B), 4.5 min-7.0 min @ 40% B, 7.0 min- 8.0 min @ 10% B; Flow rate: 2.5 mL/min; Column temperature: 40° C. |
| 116a | 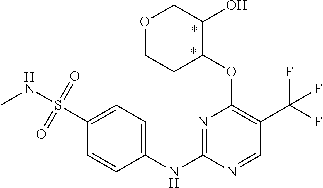 [SMILES: CNS(=O)(=O)c1ccc(Nc2ncc(C(F)(F)F)c(OC3CCOCC3O)n2)cc1] Enantiomer 1 (earlier eluting enantiomer), made from cis- tetrahydro-2H-pyran-3,4-diol | LC-MS (ESI): m/z 449.2 [M + H]⁺; ¹H NMR (400 MHz, DMSO-d₆) δ 10.50 (s, 1H), 8.60 (s, 1H), 7.93 (d, J = 8.8 Hz, 2H), 7.74 (d, J = 8.8 Hz, 2H), 7.31 (s, 1H), 5.64-5.54 (m, 1H), 5.07 (d, J = 4.8 Hz, 1H), 3.95-3.85 (m, 1H), 3.60-3.54 (m, 4H), 2.41 (d, J = 4.8 Hz, 3H), 2.09-1.98 (m, 1H), 1.92- 1.79 (m, 1H). ee: 100% Retention time: 3.31 min; Column: ChiralPak IC, 250 × 4.6 mm I.D., 5 μm, Mobile phase: A for CO₂ and B for MeOH (0.05% DEA), Gradient: 8 min @ B 40%, |
| | | Flow rate: 2.0 mL/min, Back |
| | | pressure: 100 bar, Column |
| | | temperature: 35° C. |
| 116b | 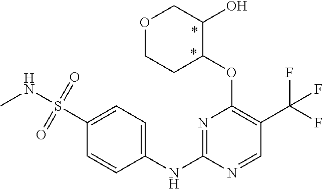 [SMILES: CNS(=O)(=O)c1ccc(Nc2ncc(C(F)(F)F)c(OC3CCOCC3O)n2)cc1] Enantiomer 2 (later eluting enantiomer), made from cis- tetrahydro-2H-pyran-3,4-diol | LC-MS (ESI): m/z 449.2 [M + H]⁺. ee: 96.5% Retention time: 4.04 min; Column: ChiralPak IC, 250 × 4.6 mm I.D., 5 μm, Mobile phase: A for CO₂ and B for MeOH (0.05% DEA), Gradient: 8 min @ B 40%, Flow rate: 2.0 mL/min, Back pressure: 100 bar, Column temperature: 35° C. |
| 117a | 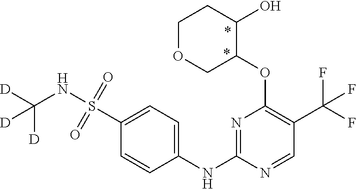 [SMILES: [2H]C([2H])([2H])NS(=O)(=O)c1ccc(Nc2ncc(C(F)(F)F)c(OC3COCCC3O)n2)cc1] Enantiomer 1 (earlier eluting enantiomer), made from cis- tetrahydro-2H-pyran-3,4-diol | LC-MS (ESI): m/z 452.2 [M + H]⁺; ¹H NMR (400 MHz, DMSO-d₆) δ 10.48 (s, 1H), 8.60 (s, 1H), 7.91 (d, J = 8.7 Hz, 2H), 7.72 (d, J = 8.8 Hz, 2H), 7.27 (s, 1H), 5.37 (s, 1H), 5.00 (d, J = 4.1 Hz, 1H), 4.13-3.74 (m, 3H), 3.65- 3.62 (m, 1H), 3.48-3.41 (m, 1H), 1.96-1.75 (m, 1H), 1.68-1.63 (m, 1H). ee: 99.3% Retention time: 3.98 min; Column: ChiralCel OD, 250 × 4.6 mm I.D., 5 μm, Mobile phase: A for CO₂ and B for MeOH (0.05% DEA), Gradient: 8 min @ B 30% Flow rate: 2.0 mL/min, Back pressure: 100 bar, Column temperature: 35° C. |
| 117b | 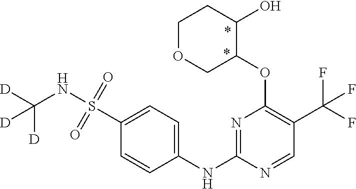 [SMILES: [2H]C([2H])([2H])NS(=O)(=O)c1ccc(Nc2ncc(C(F)(F)F)c(OC3COCCC3O)n2)cc1] Enantiomer 2 (later eluting enantiomer), made from cis- tetrahydro-2H-pyran-3,4-diol | LC-MS (ESI): m/z 452.2 [M + H]⁺. ee: 94.8% Retention time: 4.51 min; Column: ChiralCel OD, 250 × 4.6 mm I.D., 5 μm, Mobile phase: A for CO₂ and B for MeOH (0.05% DEA), Gradient: 8 min @ B 30%, Flow rate: 2.0 mL/min, Back pressure: 100 bar, Column temperature: 35° C. |
| 118a | 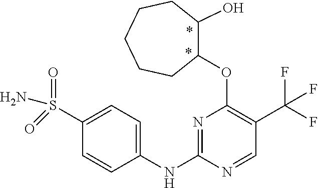 [SMILES: NS(=O)(=O)c1ccc(Nc2ncc(C(F)(F)F)c(OC3CCCCCC3O)n2)cc1] Enantiomer 1 (earlier eluting enantiomer), made from cis- cycloheptane-1,2-diol | LC-MS (ESI): m/z 447.1 [M + H]⁺; ¹H NMR (400 MHz, DMSO-d₆) δ 10.42 (br s, 1H), 8.57 (s, 1H), 7.89 (d, J = 8.8 Hz, 2H), 7.76 (d, J = 8.8 Hz, 2H), 7.23 (s, 2H), 5.51 (d, J = 7.2 Hz, 1H), 4.72 (d, J = 4.4 Hz, 1H), 3.97-3.91 (m, 1H), 2.11-2.02 (m, 1H), 1.85- 1.42 (m, 9H). ee: 99.1% Retention time: 3.03 min. Column: ChiralPak AD, 250 × 4.6 mm I.D., 5 μm, Mobile phase: A for CO₂ and B for methanol (0.05% DEA), Gradient: 10 min @ 40%. Flow rate: 2.0 mL/min, |
| | | Back pressure: 100 bar, Column |
| | | temperature: 35° C. |
| 118b | 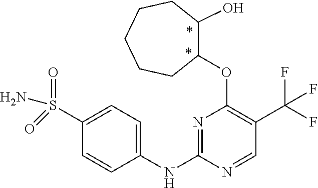 [SMILES: NS(=O)(=O)c1ccc(Nc2ncc(C(F)(F)F)c(OC3CCCCCC3O)n2)cc1] Enantiomer 2 (later eluting enantiomer), made from cis- cycloheptane-1,2-diol | LC-MS (ESI): m/z 447.2 [M + H]⁺. ee: 95.1% Retention time: 3.71 min; Column: ChiralPak AD, 250 × 4.6 mm I.D., 5 μm, Mobile phase: A for CO₂ and B for MeOH (0.05% DEA), Gradient: 8 min @ B 30%, Flow rate: 2.0 mL/min, Back pressure: 100 bar, Column temperature: 35° C. |
| 119a | 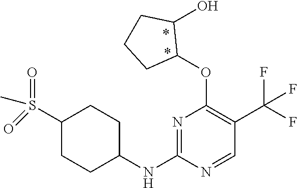 [SMILES: CS(=O)(=O)C1CCC(Nc2ncc(C(F)(F)F)c(OC3CCCC3O)n2)CC1] Enantiomer 1 (earlier eluting enantiomer), from cis-cyclopentane 1,2-diol | LC-MS (ESI): m/z 425.1 [M + H]⁺. ee: 93.1% Retention time: 4.03 min, Column: ChiralPak IC, 250 × 4.6 mm I.D., 5 μm, Mobile phase: A for CO₂ and B for MeOH (0.05% DEA), Gradient: 8 min @ B 30%, Flow rate: 2.0 mL/min, Back pressure: 100 bar, Column temperature: 35° C. |
| 119b | 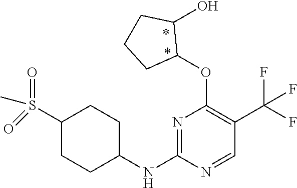 [SMILES: CS(=O)(=O)C1CCC(Nc2ncc(C(F)(F)F)c(OC3CCCC3O)n2)CC1] Enantiomer 2 (later eluting enantiomer), from cis-cyclopentane 1,2-diol | LC-MS (ESI): m/z 425.1 [M + H]⁺; ¹H NMR (400 MHz, DMSO-d₆)(tautomer ratio = 1:1) δ 8.31 & 8.27 (s, 1H), 7.91 & 7.75 (d, J = 8.0 Hz, 1H), 5.30-5.22 (m, 1H), 4.59 (d, J = 4.6 Hz, 1H), 4.22-4.15 (m, 1H), 4.0-3.75 (m, 1H), 3.55-3.52 (m, 2H), 2.92-2.80 (m, 5H), 2.22-1.89 (m, 3H), 1.88- 1.69 (m, 3H), 1.67-1.45 (m, 4H). ee: 91.2% Retention time: 4.66 min; Column: ChiralPak IC, 250 × 4.6 mm I.D., 5 μm, Mobile phase: A for CO₂ and B for MeOH (0.05% DEA), Gradient: 8 min @ 30%, |
| | | Flow rate: 2.0 mL/min, Back |
| | | pressure: 100 bar, Column |
| | | temperature: 35° C. |
| 120a | 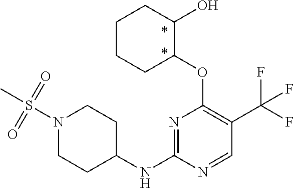 [SMILES: CS(=O)(=O)N1CCC(Nc2ncc(C(F)(F)F)c(OC3CCCCC3O)n2)CC1] Enantiomer 1 (earlier eluting enantiomer), made from cis- cyclohexane-1,2-diol | LC-MS (ESI): m/z 439.2 [M + H]⁺; ¹H NMR (400 MHz, DMSO-d₆)(tautomer ratio = 1:1) δ 8.32 & 8.28 (s, 1H), 7.94 & 7.76 (d, J = 8.0 Hz, 1H), 5.49-5.24 (m, 1H), 4.62 (s, 1H), 4.00-3.68 (m, 2H), 3.57-3.48 (m, 2H), 2.91-2.80 (m, 5H), 2.03-1.83 (m, 3H), 1.73- 1.48 (m, 6H), 1.43-1.25 (m, 3H). ee: 100% Retention time: 3.508 min; Column: ChiralPak IC, 250 × 4.6 mm I.D., 5 μm, Mobile phase: A for CO₂ and B for IPA (0.05% DEA), Gradient: 8 min @ B 40%, Flow rate: 2.0 mL/min, Back |
| | | pressure: 100 bar, Column |
| | | temperature: 35° C. |
| 120b | 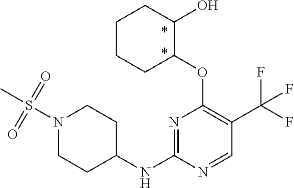 [SMILES: CS(=O)(=O)N1CCC(Nc2ncc(C(F)(F)F)c(OC3CCCCC3O)n2)CC1] Enantiomer 2 (later eluting enantiomer), made from cis- cyclohexane-1,2-diol | LC-MS (ESI): m/z 439.2 [M + H]⁺. ee: 97.0% Retention time: 4.352 min; Column: ChiralPak IC, 250 × 4.6 mm I.D., 5 μm, Mobile phase: A for CO₂ and B for IPA (0.05% DEA), Gradient: 8 min @ B 40%, Flow rate: 2.0 mL/min, Back pressure: 100 bar, Column temperature: 35° C. |
| 121a | 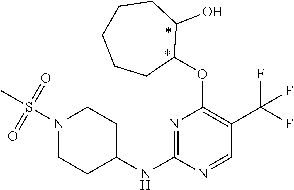 [SMILES: CS(=O)(=O)N1CCC(Nc2ncc(C(F)(F)F)c(OC3CCCCCC3O)n2)CC1] Enantiomer 1 (earlier eluting enantiomer), made from cis- cycloheptane-1,2-diol | LC-MS (ESI): m/z 453.2 [M + H]⁺; ¹H NMR (400 MHz, DMSO-d₆)(tautomer ratio = 1:1) δ 8.33 & 8.30 (s, 1H), 7.98 & 7.78 (d, J = 8.0 Hz, 1H), 5.46- 5.38 (m, 1H), 4.63 (s, 1H), 3.94-3.81 (m, 2H), 3.57-3.51 (m, 2H), 2.89-2.80 (m, 5H), 2.10-1.88 (m, 3H), 1.86- 1.40 (m, 11H). ee: 99.0% Retention time: 3.85 min; Column: ChiralPAK IC, 250 × 21.2 mm I.D., 5 μm; Mobile phase: A for CO₂ and B for IPA (0.05% DEA); Gradient: 10 min @ 40%; Flow rate: 40 mL/min; |
| | | Column temperature: 35° C. |
| 121b | 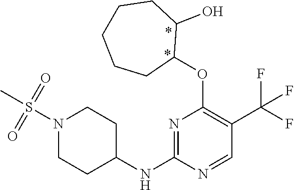 [SMILES: CS(=O)(=O)N1CCC(Nc2ncc(C(F)(F)F)c(OC3CCCCCC3O)n2)CC1] Enantiomer 2 (later eluting enantiomer), made from cis- cycloheptane-1,2-diol | LC-MS (ESI): m/z 453.2 [M + H]⁺. ee: 97.0% Retention time: 4.77 minColumn: ChiralPAK IC, 250 × 21.2 mm I.D., 5 μm; Mobile phase: A for CO₂ and B for IPA (0.05% DEA); Gradient: 10 min @ 40%; Flow rate: 40 mL/min; Column temperature: 35° C. |
| 122a | 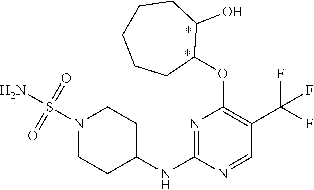 [SMILES: NS(=O)(=O)N1CCC(Nc2ncc(C(F)(F)F)c(OC3CCCCCC3O)n2)CC1] Enantiomer 1 (earlier eluting enantiomer), made from cis- cycloheptane-1,2-diol | LC-MS (ESI): m/z 454.2 [M + H]⁺. ee: 100% Retention time: 1.04 min; Column: Chiralpak IG-3, 100 × 4.6 mm I.D., 3 μm, Mobile phase: 40% of Methanol (0.05% DEA) in CO₂, Flow rate: 2.5 mL/min, Column temperature: 35° C. |
| 122b | 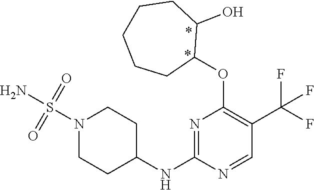 [SMILES: NS(=O)(=O)N1CCC(Nc2ncc(C(F)(F)F)c(OC3CCCCCC3O)n2)CC1] Enantiomer 2 (later eluting enantiomer), made from cis- cycloheptane-1,2-diol | LC-MS (ESI): m/z 454.2 [M + H]⁺; ¹H NMR (400 MHz, DMSO-d₆)(tautomer ratio = 1:1) δ 8.33 & 8.29 (s, 1H), 7.96 & 7.73 (d, J = 8.0 Hz, 1H), 6.76 (s, 2H), 5.43-5.37 (m, 1H), 4.69-4.58 (m, 1H), 3.98-3.60 (m, 2H), 3.47-3.32 (m, 2H), 2.68-2.56 (m, 2H), 2.12- 1.89 (m, 3H), 1.82-1.71 (m, 1H), 1.68-1.41 (m, 10H). ee: 100% Retention time: 1.33 min; Column: Chiralpak ID-3, 100 × 4.6 mm I.D., 3 μm, Mobile phase: 40% of Methanol (0.05% DEA) in CO₂, Flow rate: 2.5 mL/min, |
| | | Column temperature: 35° C. |
| 123a | 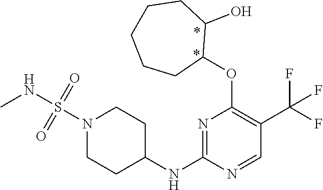 [SMILES: CNS(=O)(=O)N1CCC(Nc2ncc(C(F)(F)F)c(OC3CCCCCC3O)n2)CC1] Enantiomer 1 (earlier eluting enantiomer), made from cis- cycloheptane-1,2-diol | LC-MS (ESI): m/z 468.2 [M + H]⁺. ee: 100% Retention time: 2.50 min; Column: ChiralPak AD, 250 × 4.6 mm I.D., 5 μm, Mobile phase: A for CO₂ and B for methanol (0.05% DEA), Gradient: 10 min @ 40%, Flow rate: 2.0 mL/min, Back pressure: 100 bar, Column temperature: 35° C. |
| 123b | 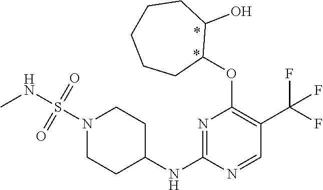 [SMILES: CNS(=O)(=O)N1CCC(Nc2ncc(C(F)(F)F)c(OC3CCCCCC3O)n2)CC1] Enantiomer 2 (later eluting enantiomer), made from cis- cycloheptane-1,2-diol | LC-MS (ESI): m/z 468.3 [M + H]⁺; ¹H NMR (400 MHz, DMSO-d₆)(tautomer ratio = 1:1) δ 8.33 & 8.29 (s, 1H), 7.97 & 7.76 (d, J = 8.0 Hz, 1H), 7.09-7.05 (m, 1H), 5.44-5.37 (m, 1H), 4.62 (s, 1H), 3.99-3.67 (m, 2H), 3.53-3.49 (m, 2H), 2.85-2.76 (m, 2H), 2.53 (s, 3H), 2.01-1.68 (m, 4H), 1.73- 1.44 (m, 10H). ee: 96.7% Retention time: 3.63 min; Column: Chiralpak AD, 250 × 4.6 mm I.D., 5 μm, Mobile phase: A for CO₂ and B for methanol (0.05% DEA), Gradient: 10 min |
| | | @ 40%, Flow rate: 2.0 mL/min, |
| | | Back pressure: 100 bar, Column |
| | | temperature: 35° C. |
| 124a | 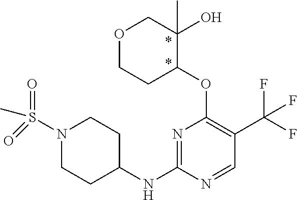 [SMILES: CC1(O)COCCC1Oc1nc(NC2CCN(S(C)(=O)=O)CC2)ncc1C(F)(F)F] Enantiomer 1 (earlier eluting enantiomer), made from cis-3- methyltetrahydro-2H-pyran-3,4- diol | LC-MS (ESI): m/z 455.2 [M + H]⁺; ¹H NMR (400 MHz, DMSO-d₆)(tautomer ratio = 1:1) δ 8.35 & 8.31 (s, 1H), 8.00 & 7.82 (d, J = 6.8 Hz, 1H), 5.23-5.12 (m, 1H), 4.53 (s, 1H), 4.20-3.70 (m, 2H), 3.59-3.46 (m, 4H), 3.29- 3.21 (m, 1H), 2.90-2.79 (m, 5H), 1.99-1.78 (m, 4H), 1.65-1.45 (m, 2H), 1.12 (s, 3 H). ee: 100% Retention time: 3.70 min; Column: ChiralPak C-IG, 100 × 4.6 mm I.D., 5 μm; Mobile phase: A for CO₂ and B for methanol (0.05% DEA); Gradient: 0.0 min- 1.0 min @10% B, 1.0 min-4.5 |
| | | min gradient (10-40% B), 4.5 |
| | | min-7.0 min @ 40% B, 7.0 min- |
| | | 8.0 min @ 10% B; Flow rate: 2.5 |
| | | mL/min; Column temperature: |
| | | 40° C. |
| 124b | 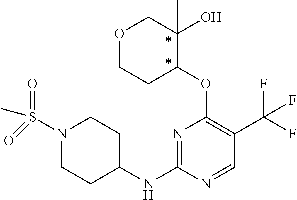 [SMILES: CC1(O)COCCC1Oc1nc(NC2CCN(S(C)(=O)=O)CC2)ncc1C(F)(F)F] Enantiomer 2 (later eluting enantiomer), made from cis-3- methyltetrahydro-2H-pyran-3,4- diol | LC-MS (ESI): m/z 455.2 [M + H]⁺. ee: 100% Retention time: 4.33 min; Column: ChiralPak C-IG, 100 × 4.6 mm I.D., 5 μm; Mobile phase: A for CO₂ and B for methanol (0.05% DEA); Gradient: 0.0 min- 1.0 min @ 10% B, 1.0 min-4.5 min gradient (10-40% B), 4.5 min-7.0 min @ 40% B, 7.0 min- 8.0 min @ 10% B; Flow rate: 2.5 mL/min; Column temperature: 40° C. |
| 125a | 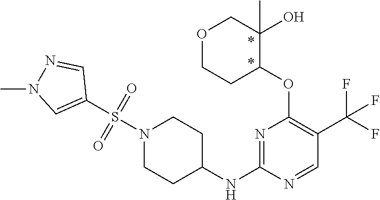 [SMILES: Cn1cc(S(=O)(=O)N2CCC(Nc3ncc(C(F)(F)F)c(OC4CCOCC4(C)O)n3)CC2)cn1] Enantiomer 1 (earlier eluting enantiomer), made from cis-3- methyltetrahydro-2H-pyran-3,4- diol | LC-MS (ES): m/z 521.1 [M + H]⁺; ¹H NMR (400 MHz, DMSO-d₆)(tautomer ratio = 1:1) δ 8.32-8.27 (m, 2H), 7.96-7.76 (m, 2H), 5.20-5.06 (m, 1H), 4.54 & 4.51 (s, 1H), 3.90 (s, 3H), 3.68- 3.47 (m, 5H), 3.27-3.25 (m, 2H), 2.50-2.49 (m, 2H), 1.92-1.87 (m, 4H), 1.86-1.53 (m, 2H), 1.09 & 1.08 (s, 3H). ee: 100% Retention time: 2.25 min; Column: ChiralPak C-IG, 100 × 4.6 mm I.D., 5 μm; Mobile phase: A for CO₂ and B for methanol (0.05% DEA); Gradient: 8 min @ 40% B; Flow rate: 2.5 mL/min; Column temperature: 40° C. |
| 125b | 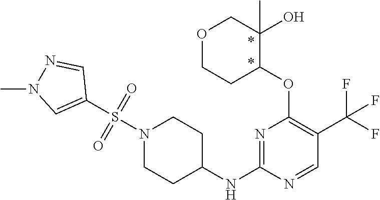 [SMILES: Cn1cc(S(=O)(=O)N2CCC(Nc3ncc(C(F)(F)F)c(OC4CCOCC4(C)O)n3)CC2)cn1] Enantiomer 2 (later eluting enantiomer), made from cis-3- methyltetrahydro-2H-pyran-3,4- diol | LC-MS (ESI): m/z 521.1 [M + H]⁺. ee: 100% Retention time: 3.33 min; Column: ChiralPak C-IG, 100 × 4.6 mm I.D., 5 μm; Mobile phase: A for CO₂ and B for methanol (0.05% DEA); Gradient: 8 min @ 40% B; Flow rate: 2.5 mL/min; Column temperature: 40° C. |
| 126 | 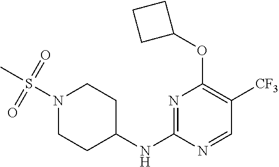 [SMILES: CS(=O)(=O)N1CCC(Nc2ncc(C(F)(F)F)c(OC3CCC3)n2)CC1] | LC-MS (ESI): m/z 395.1 [M + H]⁺; ¹H NMR (500 MHz, DMSO-d₆) δ 8.34 & 8.30 (s, 1H), 8.00 & 7.81 (d, J = 7.8 Hz, 1H), 5.38-4.94 (m, 1H), 3.97-3.72 (m, 1H), 3.54 (d, J = 12.2 Hz, 2H), 3.00-2.74 (m, 5H), 2.46-2.39 (m, 2H), 2.19-2.01 (m, 2H), 2.00- 1.89 (m, 2H), 1.87-1.77 (m, 1H), 1.75-1.61 (m, 1H), 1.61- 1.47 (m, 2H). |
| 127 | 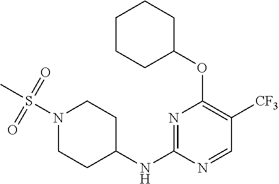 [SMILES: CS(=O)(=O)N1CCC(Nc2ncc(C(F)(F)F)c(OC3CCCCC3)n2)CC1] | LC-MS (ESI): m/z 423.2 [M + H]⁺; ¹H NMR (500 MHz, DMSO-d₆) δ 8.33 & 8.29 (s, 1H), 7.98 & 7.80 (d, J = 7.5 Hz, 1H), 5.33-5.07 (m, 1H), 3.98-3.72 (m, 1H), 3.54 (d, J = 12.0 Hz, 2H), 2.90-2.78 (m, 5H), 2.06-1.79 (m, 4H), 1.74-1.33 (m, 10H). |
| 128 | 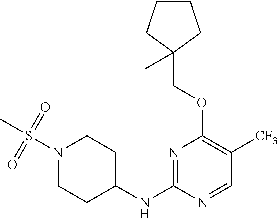 [SMILES: CC1(COc2nc(NC3CCN(S(C)(=O)=O)CC3)ncc2C(F)(F)F)CCCC1] | LC-MS (ESI): m/z 437.2 [M + H]⁺; ¹H NMR (500 MHz, DMSO-d₆) δ 8.34 & 8.30 (s, 1H), 8.00 & 7.88 (d, J = 7.8 Hz, 1H), 4.17 & 4.11 (s, 2H), 4.00-3.81 (m, 1H), 3.53 (d, J = 12.5 Hz, 2H), 2.98-2.75 (m, 5H), 2.00-1.89 (m, 2H), 1.76-1.47 (m, 8H), 1.37- 1.28 (m, 2H), 1.04 (s, 3H). |
| 129 | 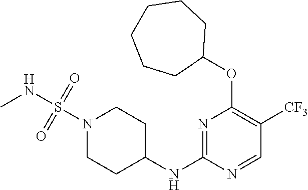 [SMILES: CNS(=O)(=O)N1CCC(Nc2ncc(C(F)(F)F)c(OC3CCCCCC3)n2)CC1] | LC-MS (ESI): m/z 452.2 [M + H]⁺; ¹H NMR (500 MHz, DMSO-d₆) δ 8.33 & 8.29 (s, 1H), 7.99 & 7.78 (d, J = 7.5 Hz, 1H), 7.11-7.04 (m, 1H), 5.47-5.23 (m, 1H), 4.00-3.73 (m, 1H), 3.65- 3.42 (m, 2H), 2.81 (t, J = 12.0 Hz, 2H), 2.53 (d, J = 5.0 Hz, 3H), 2.04-1.86 (m, 4H), 1.82-1.74 (m, 2H), 1.69-1.45 (m, 10H). |
| 136a | 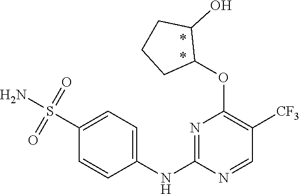 [SMILES: NS(=O)(=O)c1ccc(Nc2ncc(C(F)(F)F)c(OC3CCCC3O)n2)cc1] Enantiomer 1 (earlier eluting enantiomer), from cis-cyclopentane 1,2-diol | LC-MS (ESI): m/z 385.1 [M + H]⁺; ¹H NMR (400 MHz, DMSO-d₆) δ 10.01 (s, 1H), 8.34 (s, 1H), 7.85 (d, J = 8.8 Hz, 2H), 7.72 (d, J = 8.8 Hz, 2H), 7.18 (s, 2H), 5.31-5.21 (m, 1H), 4.74 (d, J = 4.8 Hz, 1H), 4.3-4.22 (m, 1H), 2.07-2.02 (m, 1H), 1.87-1.81 (m, 3H), 1.69-1.63 (m, 1H), 1.58-1.48 (s, 1H). ee: 96.3% Retention time: 4.27 min; Column: ChiralCel OD, 250 × 21.2 mm I.D., 5 μm; Mobile phase: A for CO₂ and B for methanol (0.05% DEA); |
| | | Gradient: 10 min @ 40%; Flow |
| | | rate: 2.0 mL/min; Column |
| | | temperature: 35° C. |
| 136b | 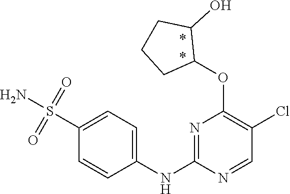 [SMILES: NS(=O)(=O)c1ccc(Nc2ncc(Cl)c(OC3CCCC3O)n2)cc1] Enantiomer 2 (later eluting enantiomer), from cis-cyclopentane 1,2-diol | LC-MS (ESI): m/z 385.1 [M + H]⁺; ¹H NMR (400 MHz, DMSO-d₆) δ 10.02 (s, 1H), 8.34 (s, 1H), 7.85 (d, J = 8.8 Hz, 2H), 7.73 (d, J = 8.8 Hz, 2H), 7.18 (s, 2H), 5.28-5.24 (m, 1H), 4.73 (d, J = 4.8 Hz, 1H), 4.28-4.25 (m, 1H), 2.07-2.02 (m, 1H), 1.87-1.76 (m, 3H), 1.70-1.65 (m, 1H), 1.56-1.52 (s, 1H). ee: 53.5% Retention time: 5.58 min; Column: ChiralCel OD, 250 × 21.2 mm I.D., 5 μm; Mobile phase: A for CO₂ and B for methanol (0.05% DEA); |
| | | Gradient: 10 min @ 40%; Flow |
| | | rate: 2.0 mL/min; Column |
| | | temperature: 35° C. |
| 137a | 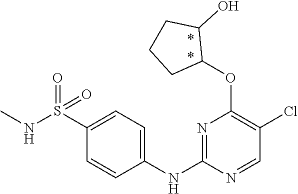 [SMILES: CNS(=O)(=O)c1ccc(Nc2ncc(Cl)c(OC3CCCC3O)n2)cc1] Enantiomer 1 (earlier eluting enantiomer), from cis-cyclopentane 1,2-diol | LC-MS (ESI): m/z 399.1 [M + H]⁺; ¹H NMR (400 MHz, DMSO-d₆) δ 10.08 (s, 1H), 8.36 (s, 1H), 7.92 (d, J = 8.8 Hz, 2H), 7.70 (d, J = 8.8 Hz, 2H), 7.26 (q, J = 9.6 Hz, 1H), 5.29-5.25 (m, 1H), 4.73 (d, J = 4.8 Hz, 1H), 4.29-4.24 (m, 1H), 2.40 (d, J = 4.8 Hz, 3H), 2.08-1.99 (m, 1H), 1.89- 1.75 (m, 3H), 1.70-1.61 (m, 1H), 1.58-1.49 (m, 1H). ee: 97.9% Retention time: 6.40 min; Column: ChiralPak IC, 250 × 4.6 mm I.D., 5 μm, Mobile phase: A for CO₂ and B for MeOH (0.05% |
| | | DEA), Gradient: 8 min @ B 40%, |
| | | Flow rate: 1.8 mL/min, Back |
| | | pressure: 100 bar, Column |
| | | temperature: 35° C. |
| 137b | 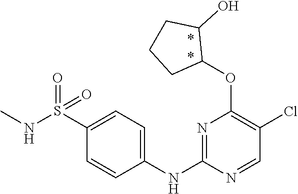 [SMILES: CNS(=O)(=O)c1ccc(Nc2ncc(Cl)c(OC3CCCC3O)n2)cc1] Enantiomer 2 (later eluting enantiomer), from cis-cyclopentane 1,2-diol | LC-MS (ESI): m/z 399.1 [M + H]⁺. ee: 96.1% Retention time: 8.89 min; Column: ChiralPak IC, 250 × 4.6 mm I.D., 5 μm, Mobile phase: A for CO₂ and B for MeOH (0.05% DEA), Gradient: 8 min @ B 40%, Flow rate: 1.8 mL/min, Back pressure: 100 bar, Column temperature: 35° C. |
| 138a | 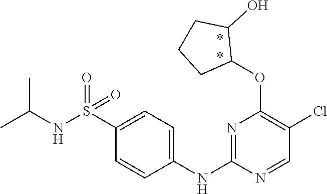 [SMILES: CC(C)NS(=O)(=O)c1ccc(Nc2ncc(Cl)c(OC3CCCC3O)n2)cc1] Enantiomer 1 (earlier eluting enantiomer), from cis-cyclopentane 1,2-diol | LC-MS (ESI): m/z 427.0 [M + H]⁺; ¹H NMR (400 MHz, DMSO-d6) δ 10.06 (s, 1H), 8.35 (s, 1H), 7.89 (d, J = 8.8 Hz, 2H), 7.72 (d, J = 8.8 Hz, 2H), 7.38 (d, J = 7.2 Hz, 1H), 5.28-5.24 (m, 1H), 4.72 (d, J = 4.8 Hz, 1H), 4.28-4.23 (m, 1H), 3.24-3.16 (m, 1H), 2.07-2.01 (m, 1H), 1.89-1.76 (m, 3H), 1.70-1.61 (m, 2H), 0.95 (d, J = 6.4 Hz, 6H). ee: 98.1% Retention time: 3.16 min; Column: ChiralCel OD, 250 × 4.6 mm I.D., 5 μm, Mobile phase: A for CO₂ and B for MeOH (0.05% |
| | | DEA), Gradient: 8 min @ B 40%, |
| | | Flow rate: 2.0 mL/min, Back |
| | | pressure: 100 bar, Column |
| | | temperature: 35° C. |
| 138b | 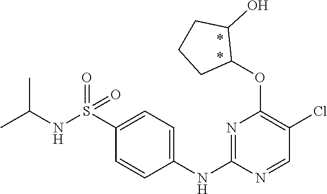 [SMILES: CC(C)NS(=O)(=O)c1ccc(Nc2ncc(Cl)c(OC3CCCC3O)n2)cc1] Enantiomer 2 (later eluting enantiomer), from cis-cyclopentane 1,2-diol | LC-MS (ESI): m/z 427.0 [M + H]⁺; ¹H NMR (400 MHz, DMSO-d₆) δ 10.06 (s, 1H), 8.35 (s, 1H), 7.89 (d, J = 8.8 Hz, 2H), 7.72 (d, J = 8.8 Hz, 2H), 7.38 (d, J = 7.2 Hz, 1H), 5.28-5.24 (m, 1H), 4.72 (d, J = 4.8 Hz, 1H), 4.28-4.24 (m, 1H), 3.24-3.16 (m, 1H), 2.06-2.00 (m, 1H), 1.89-1.75 (m, 3H), 1.70-1.61 (m, 2H), 0.95 (d, J = 6.4 Hz, 6H). ee: 97.4% Retention time: 4.02 min; Column: ChiralCel OD, 250 × 4.6 mm I.D., 5 μm, Mobile phase: A for CO₂ and B for MeOH (0.05% |
| | | DEA), Gradient: 8 min @ B 40%, |
| | | Flow rate: 2.0 mL/min, Back |
| | | pressure: 100 bar, Column |
| | | temperature: 35° C. |
| 139a | 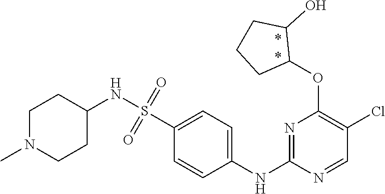 [SMILES: CN1CCC(NS(=O)(=O)c2ccc(Nc3ncc(Cl)c(OC4CCCC4O)n3)cc2)CC1] Enantiomer 1 (earlier eluting enantiomer), from cis-cyclopentane 1,2-diol | LC-MS (ESI): m/z 482.2 [M + H]⁺; ¹H NMR (400 MHz, DMSO-d₆) δ 10.07 (s, 1H), 8.35 (s, 1H), 7.87 (d, J = 8.8 Hz, 2H), 7.71 (d, J = 8.8 Hz, 2H), 7.53 (d, J = 5.4 Hz, 1H), 5.28-5.24 (m, 1H), 4.72 (d, J = 4.8 Hz, 1H), 4.32-4.20 (m, 1H), 2.95-2.84 (m, 1H), 2.70-2.58 (m, 2H), 2.15 (s, 3H), 2.09-1.91 (m, 3H), 1.88-1.78 (m, 3H), 1.72-1.62 (m, 1H), 1.60- 1.50 (m, 3H), 1.45-1.35 (m, 2H). ee: 95.5% Retention time: 3.18 min; Column: ChiralCel OD, 250 × 4.6 mm I.D., 5 μm, Mobile phase: A |
| | | for CO₂ and B for MeOH (0.05% |
| | | DEA), Gradient: 8 min @ B 40%, |
| | | Flow rate: 2.0 mL/min, Back |
| | | pressure: 100 bar, Column |
| | | temperature: 35° C. |
| 139b | 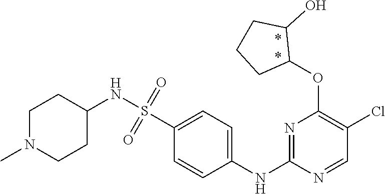 [SMILES: CN1CCC(NS(=O)(=O)c2ccc(Nc3ncc(Cl)c(OC4CCCC4O)n3)cc2)CC1] Enantiomer 2 (later eluting enantiomer), from cis-cyclopentane 1,2-diol | LC-MS (ESI): m/z 482.2 [M + H]⁺; ¹H NMR (400 MHz, DMSO-d₆) δ 10.07 (s, 1H), 8.35 (s, 1H), 7.88 (d, J = 8.8 Hz, 2H), 7.71 (d, J = 8.8 Hz, 2H), 7.55 (d, J = 6.8 Hz, 1H), 5.28-5.24 (m, 1H), 4.72 (d, J = 4.8 Hz, 1H), 4.32-4.20 (m, 1H), 2.95-2.84 (m, 1H), 2.70-2.58 (m, 2H), 2.20 (s, 3H), 2.10-1.97 (m, 3H), 1.90-1.77 (m, 3H), 1.72-1.64 (m, 1H), 1.61- 1.50 (m, 3H), 1.47-1.35 (m, 2H). ee: 94.3% Retention time: 4.23 min; Column: ChiralCel OD, 250 × 4.6 mm I.D., 5 μm, Mobile phase: A |
| | | for CO₂ and B for MeOH (0.05% |
| | | DEA), Gradient: 8 min @ B 40%, |
| | | Flow rate: 2.0 mL/min, Back |
| | | pressure: 100 bar, Column |
| | | temperature: 35° C. |
| 140a | 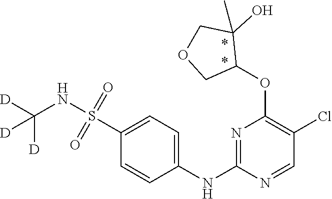 [SMILES: [2H]C([2H])([2H])NS(=O)(=O)c1ccc(Nc2ncc(Cl)c(OC3COCC3(C)O)n2)cc1] Enantiomer 1 (earlier eluting enantiomer), from 3- methyltetrahydrofuran-3,4-diol | LC-MS (ESI): m/z 418.1 [M + H]⁺; ¹H NMR (400 MHz, DMSO-d₆) δ 10.08 (s, 1H), 8.37 (s, 1H), 7.89 (d, J = 8.8 Hz, 2H), 7.69 (d, J = 8.8 Hz, 2H), 7.22 (s, 1H), 5.34-5.30 (m, 1H), 5.05 (s, 1H), 4.27-4.21 (m, 1H), 3.85-3.80 (m, 1H), 3.64 (d, J = 8.2 Hz, 1H), 3.55 (d, J = 8.2 Hz, 1H), 1.36 (s, 3H). ee: 99.8% Retention time: 2.34 min; Column: Chiralpak AD-3, 150 × 4.6 mm I.D., 3 μm, Mobile phase: 40% of ethanol (0.05% DEA) in CO₂, Flow rate: 2.5 mL/min, |
| | | Column temperature: 35° C. |
| 140b | 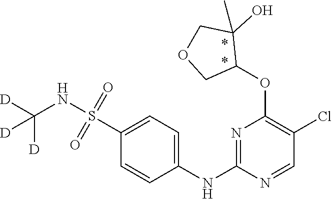 [SMILES: [2H]C([2H])([2H])NS(=O)(=O)c1ccc(Nc2ncc(Cl)c(OC3COCC3(C)O)n2)cc1] Enantiomer 2 (later eluting enantiomer), from 3- methyltetrahydrofuran-3,4-diol | LC-MS (ESI): m/z 418.1 [M + H]⁺. ee: 98.7% Retention time: 3.63 min; Column: Chiralpak AD-3, 150 × 4.6 mm I.D., 3 μm, Mobile phase: 40% of ethanol (0.05% DEA) in CO₂, Flow rate: 2.5 mL/min, Column temperature: 35° C. |
| 141a | 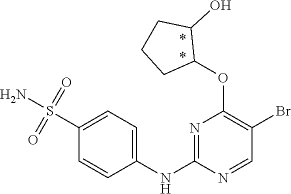 [SMILES: NS(=O)(=O)c1ccc(Nc2ncc(Br)c(OC3CCCC3O)n2)cc1] Enantiomer 1 (earlier eluting enantiomer), from cis-cyclopentane 1,2-diol | LC-MS (ESI): m/z 429.0, 431.0 [M + H]⁺; ¹H NMR (400 MHz, DMSO-d₆) δ 10.02 (s, 1H), 8.41 (s, 1H), 7.85 (d, J = 8.4 Hz, 2H), 7.74 (d, J = 8.4 Hz, 2H), 7.18 (s, 2H), 5.29-5.22 (m, 1H), 4.71 (d, J = 4.8 Hz, 1H), 4.32-4.20 (m, 1H), 2.05-1.89 (m, 1H), 1.87-1.77 (m, 3H), 1.71-1.61 (m, 1H), 1.57-1.53 (m, 1H). ee: 90.7% Retention time: 4.67 min; Column: ChiralCel OD, 250 × 21.2 mm I.D., 5 μm; Mobile phase: A for CO₂ and B for methanol (0.05% DEA); |
| | | Gradient: 10 min @ 40%; Flow |
| | | rate: 2.0 mL/min; Column |
| | | temperature: 35° C. |
| 141b | 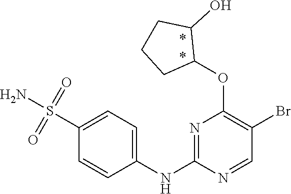 [SMILES: NS(=O)(=O)c1ccc(Nc2ncc(Br)c(OC3CCCC3O)n2)cc1] Enantiomer 2 (later eluting enantiomer), from cis-cyclopentane 1,2-diol | LC-MS (ESI): m/z 429.1, 431.1 [M + H]⁺; ¹H NMR (400 MHz, DMSO-d₆) δ 10.02 (s, 1H), 8.41 (s, 1H), 7.85 (d, J = 8.8 Hz, 2H), 7.73 (d, J = 8.8 Hz, 2H), 7.17 (s, 2H), 5.29-5.22 (m, 1H), 4.71 (d, J = 4.8 Hz, 1H), 4.33-4.19 (m, 1H), 2.10-1.98 (m, 1H), 1.90-1.77 (m, 3H), 1.72-1.66 (m, 1H), 1.59- 1.52 (m, 1H). ee: 94.2% Retention time: 6.56 min; Column: ChiralCel OD, 250 × 21.2 mm I.D., 5 μm; Mobile phase: A for CO₂ and B for methanol (0.05% DEA); |
| | | Gradient: 10 min @ 40%; Flow |
| | | rate: 2.0 mL/min; Column |
| | | temperature: 35° C. |
| 142a | 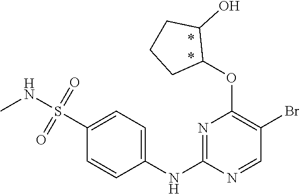 [SMILES: CNS(=O)(=O)c1ccc(Nc2ncc(Br)c(OC3CCCC3O)n2)cc1] Enantiomer 1 (earlier eluting enantiomer), from cis-cyclopentane 1,2-diol | LC-MS (ESI): m/z 443.1 & 445.1 [M + H]⁺; ¹H NMR (400 MHz, DMSO-d₆) δ δ 10.08 (s, 1H), 8.42 (s, 1H), 7.90 (d, J = 8.8 Hz, 2H), 76.8 (d, J = 8.8 Hz, 2H), 7.26 (q, J = 9.6 Hz, 1H), 5.31-5.20 (m, 1H), 4.71 (d, J = 4.8 Hz, 1H), 4.34-4.16 (m, 1H), 2.39 (d, J = 4.8 Hz, 3H), 2.05-1.95 (m, 1H), 1.91- 1.75 (m, 3H), 1.72-1.47 (m, 2H). ee: 98.5% Retention time: 7.12 min; Column: ChiralPAK IC, 250 × 21.2 mm I.D., 5 μm; Mobile phase: A for CO₂ and B for methanol (0.05% DEA); |
| | | Gradient: 10 min @ 40%; Flow |
| | | rate: 2.0 mL/min; Column |
| | | temperature: 35° C. |
| 142b | 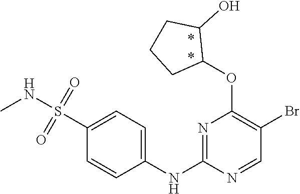 [SMILES: CNS(=O)(=O)c1ccc(Nc2ncc(Br)c(OC3CCCC3O)n2)cc1] Enantiomer 2 (later eluting enantiomer), from cis-cyclopentane 1,2-diol | LC-MS (ESI): m/z 443.1 & 445.1 [M + H]⁺. ee: 8.14% Retention time: 10.56 min; Column: ChiralPAK IC, 250 × 21.2 mm I.D., 5 μm; Mobile phase: A for CO₂ and B for methanol (0.05% DEA); Gradient: 10 min @ 40%; Flow rate: 2.0 mL/min; Column temperature: 35° C. |
| 143a | 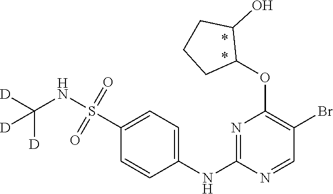 [SMILES: [2H]C([2H])([2H])NS(=O)(=O)c1ccc(Nc2ncc(Br)c(OC3CCCC3O)n2)cc1] Enantiomer 1 (earlier eluting enantiomer), from cis-cyclopentane 1,2-diol | LC-MS (ESI): m/z 446.1 & 448.1 [M + H]⁺; ¹H NMR (400 MHz, DMSO-d₆) δ 10.08 (s, 1H), 8.42 (s, 1H), 7.90 (d, J = 8.8 Hz, 2H), 76.8 (d, J = 8.8 Hz, 2H), 7.22 (s, 1H), 5.29-5.24 (m, 1H), 4.71 (d, J = 4.8 Hz, 1H), 4.32-4.17 (m, 1H), 2.03 (m, 1H), 1.91-1.74 (m, 3H), 1.73-1.52 (m, 2H). ee: 86.0% Retention time: 4.17 min; Column: ChiralCel OD, 250 × 4.6 mm I.D., 5 μm, Mobile phase: A for CO₂ and B for MeOH (0.05% DEA), Gradient: 8 min @ 40%, Flow rate: 2.0 mL/min, Back |
| | | pressure: 100 bar, Column |
| | | temperature: 35° C. |
| 143b | 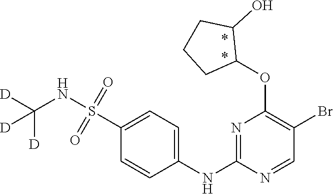 [SMILES: [2H]C([2H])([2H])NS(=O)(=O)c1ccc(Nc2ncc(Br)c(OC3CCCC3O)n2)cc1] Enantiomer 2 (later eluting enantiomer), from cis-cyclopentane 1,2-diol | LC-MS (ESI): m/z 446.1 & 448.1 [M + H]⁺. ee: 86.6% Retention time: 5.31 min; Column: ChiralCel OD, 250 × 4.6 mm I.D., 5 μm, Mobile phase: A for CO₂ and B for MeOH (0.05% DEA), Gradient: 8 min @ B 40%, Flow rate: 2.0 mL/min, Back pressure: 100 bar, Column temperature: 35° C. |
| 144a | 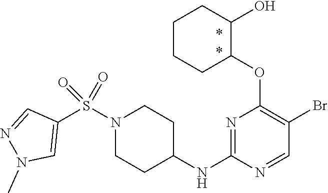 [SMILES: Cn1cc(S(=O)(=O)N2CCC(Nc3ncc(Br)c(OC4CCCCC4O)n3)CC2)cn1] Enantiomer 1 (earlier eluting enantiomer), made form cis- cyclohexane-1,2-diol | LC-MS (ESI): m/z 515.2 & 517.2 [M + H]⁺. ee: 97.0% Retention time: 6.853 min; Column: ChiralPak AD, 250 × 4.6 mm I.D., 5 μm, Mobile phase: A for CO₂ and B for isopropanol (0.05% DEA), Gradient: 10 min @ 30%, Flow rate: 2.0 mL/min, Back pressure: 100 bar, Column temperature: 35° C. |
| 144b | 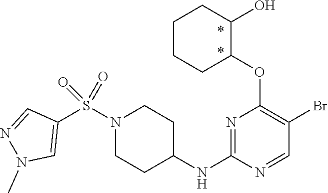 [SMILES: Cn1cc(S(=O)(=O)N2CCC(Nc3ncc(Br)c(OC4CCCCC4O)n3)CC2)cn1] Enantiomer 2 (later eluting enantiomer), made from cis- cyclohexane-1,2-diol | LC-MS (ESI): m/z 515.2 & 517.2 [M + H ]⁺; ¹H NMR (400 MHz, DMSO-d₆) δ 8.32 (s, 1H), 8.11 (s, 1H), 7.78 (s, 1H), 7.20 (s, 1H), 5.18 (s, 1H), 4.64 (d, J = 4.4 Hz, 1H), 3.91 (s, 3H), 3.80-3.72 (m, 1H), 3.66-3.54 (m, 1H), 3.51-3.44 (m, 2H), 2.45-2.36 (m, 2H), 1.96- 1.83 (m, 3H), 1.74-1.46 (m, 7H), 1.37-1.27 (m, 2H). ee: 94.4% Retention time: 7.92 min; Column: ChiralPak AD, 250 × 4.6 mm I.D., 5 μm, Mobile phase: A for CO₂ and B for isopropanol (0.05% DEA), Gradient: 10 min |
| | | @ 30%, Flow rate: 2.0 mL/min, |
| | | Back pressure: 100 bar, Column |
| | | temperature: 35° C. |
| 145a | 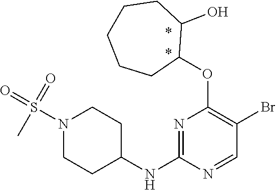 [SMILES: CS(=O)(=O)N1CCC(Nc2ncc(Br)c(OC3CCCCCC3O)n2)CC1] Enantiomer 1 (earlier eluting enantiomer), made from cis- cycloheptane-1,2-diol | LC-MS (ESI): m/z 463.1 & 465.1 [M + H]⁺; ¹H NMR (400 MHz, DMSO-d6)(tautomer ratio = 1:1) δ 8.14 (s, 1H), 7.20 & 7.35 (s, 1H), 5.27 (s, 1H), 4.66 (d, J = 4.6 Hz, 1H), 3.93-3.65 (m, 2H), 3.58- 3.48 (m, 2H), 2.88-2.79 (m, 5H), 2.02-1.80 (m, 4H), 1.69-1.45 (m, 10H). ee: 98.6% Retention time: 3.94 min; Column: ChiralPak AD, 250 × 4.6 mm I.D., 5 μm, Mobile phase: A for CO₂ and B for methanol (0.05% DEA), Gradient: 10 min @ 40%, Flow rate: 2.0 mL/min, |
| | | Back pressure: 100 bar, Column |
| | | temperature: 35° C. |
| 145b | 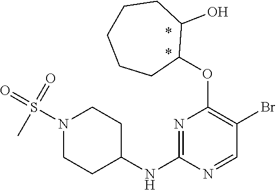 [SMILES: CS(=O)(=O)N1CCC(Nc2ncc(Br)c(OC3CCCCCC3O)n2)CC1] Enantiomer 2 (later eluting enantiomer), made from cis- cycloheptane-1,2-diol | LC-MS (ESI): m/z 463.1 & 465.1 [M + H]⁺; ¹H NMR (400 MHz, DMSO-d₆)(tautomer ratio = 1:1) δ 8.14 (s, 1H), 7.20 & 7.35 (s, 1H), 5.28 (s, 1H), 4.66 (d, J = 4.6 Hz, 1H), 3.87-3.75 (m, 2H), 3.54- 3.51 (m, 2H), 2.87-2.78 (m, 5H), 1.95-1.82 (m, 4H), 1.68-1.44 (m, 10H). ee: 90.3% Retention time: 4.79 min; Column: ChiralPak AD, 250 × 4.6 mm I.D., 5 μm, Mobile phase: A for CO₂ and B for methanol (0.05% DEA), Gradient: 10 min @ 40%, Flow rate: 2.0 mL/min, |
| | | Back pressure: 100 bar, Column |
| | | temperature: 35° C. |
| 146a | 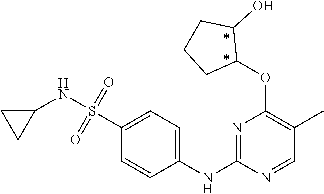 [SMILES: Cc1cnc(Nc2ccc(S(=O)(=O)NC3CC3)cc2)nc1OC1CCCC1O] Enantiomer 1 (earlier eluting enantiomer), from cis-cyclopentane 1,2-diol | LC-MS (ESI): m/z 405.2 [M + H]⁺; ¹H NMR (400 MHz, DMSO-d₆) δ 9.76 (s, 1H), 8.09 (s, 1H), 7.93 (d, J = 8.8 Hz, 2H), 7.71-7.62 (m, 3H), 5.28-5.18 (m, 1H), 4.62 (d, J = 5.0 Hz, 1H), 4.26-4.18 (m, 1H), 2.11-1.98 (m, 2H), 2.03 (s, 3H), 1.90-1.75 (m, 3H), 1.71-1.63 (m, 1H), 1.60-1.49 (m, 1H), 0.50-0.43 (m, 2H), 0.39- 0.32 (m, 2H). ee: 95.9% Retention time: 7.44 min; Column: ChiralPak IC, 250 × 4.6 mm I.D., 5 μm, Mobile phase: A for CO₂ and B for MeOH (0.05% |
| | | DEA), Gradient: 8 min @ B 40%, |
| | | Flow rate: 2.0 mL/min, Back |
| | | pressure: 100 bar, Column |
| | | temperature: 35° C. |
| 146b | 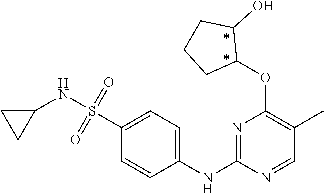 [SMILES: Cc1cnc(Nc2ccc(S(=O)(=O)NC3CC3)cc2)nc1OC1CCCC1O] Enantiomer 2 (later eluting enantiomer), from cis-cyclopentane 1,2-diol | LC-MS (ESI): m/z 405.2 [M + H]⁺; ¹H NMR (400 MHz, DMSO-d₆) δ 9.76 (s, 1H), 8.09 (s, 1H), 7.93 (d, J = 8.8 Hz, 2H), 7.71-7.64 (m, 3H), 5.31-5.14 (m, 1H), 4.62 (d, J = 5.0 Hz, 1H), 4.31-4.12 (m, 1H), 2.12-1.99 (m, 2H), 2.03 (s, 3H), 1.88-1.75 (m, 3H), 1.72-1.64 (m, 1H), 1.60-1.50 (m, 1H), 0.51-0.43 (m, 2H), 0.38- 0.31 (m, 2H). ee: 96.8% Retention time: 11.22 min; Column: ChiralPak IC, 250 × 4.6 mm I.D., 5 μm, Mobile phase: A for CO₂ and B for MeOH (0.05% |
| | | DEA), Gradient: 8 min @ B 40%, |
| | | Flow rate: 2.0 mL/min, Back |
| | | pressure: 100 bar, Column |
| | | temperature: 35° C. |
| 147a | 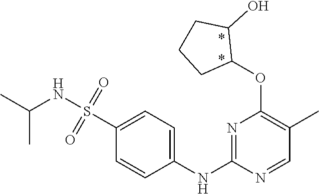 [SMILES: Cc1cnc(Nc2ccc(S(=O)(=O)NC(C)C)cc2)nc1OC1CCCC1O] Enantiomer 1 (earlier eluting enantiomer), from cis-cyclopentane 1,2-diol | LC-MS (ESI): m/z 407.2 [M + H]⁺; ¹H NMR (400 MHz, DMSO-d₆) δ 9.73 (s, 1H), 8.08 (s, 1H), 7.90 (d, J = 8.8 Hz, 2H), 7.67 (d, J = 8.8 Hz, 2H), 7.32 (s, 1H), 5.36-5.14 (m, 1H), 4.62 (d, J = 5.0 Hz, 1H), 4.35-4.03 (m, 1H), 3.26- 3.11 (m, 1H), 2.10-1.95 (m, 1H), 2.03 (s, 3H), 1.89-1.75 (m, 3H), 1.71-1.62 (m, 1H), 1.59-1.45 (m, 1H), 0.94 (d, J = 6.4 Hz, 6H). ee: 86.3% Retention time: 5.53 min; Column: ChiralPak IC, 250 × 4.6 mm I.D., 5 μm, Mobile phase: A for CO₂ and B for MeOH (0.05% |
| | | DEA), Gradient: 8 min @ B 40%, |
| | | Flow rate: 2.0 mL/min, Back |
| | | pressure: 100 bar, Column |
| | | temperature: 35° C. |
| 147b | 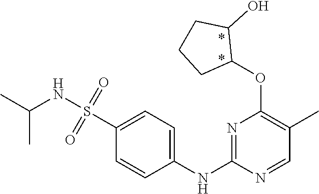 [SMILES: Cc1cnc(Nc2ccc(S(=O)(=O)NC(C)C)cc2)nc1OC1CCCC1O] Enantiomer 2 (later eluting enantiomer), from cis-cyclopentane 1,2-diol | LC-MS (ESI): m/z 407.2 [M + H]⁺; ¹H NMR (400 MHz, DMSO-d₆) δ 9.73 (s, 1H), 8.08 (s, 1H), 7.90 (d, J = 8.8 Hz, 2H), 7.67 (d, J = 8.8 Hz, 2H), 7.32 (s, 1H), 5.31-5.16 (m, 1H), 4.62 (d, J = 5.0 Hz, 1H), 4.26-4.13 (m, 1H), 3.24- 3.14 (m, 1H), 2.07-1.98 (m, 1H), 2.03 (s, 3H), 1.88-1.75 (m, 3H), 1.72-1.62 (m, 1H), 1.59-1.50 (m, 1H), 0.94 (d, J = 6.4 Hz, 6H). ee: 86.3% Retention time: 9.33 min; Column: ChiralPak IC, 250 × 4.6 mm I.D., 5 μm, Mobile phase: A for CO₂ and B for MeOH (0.05% |
| | | DEA), Gradient: 8 min @ B 40%, |
| | | Flow rate: 2.0 mL/min, Back |
| | | pressure: 100 bar, Column |
| | | temperature: 35° C. |
| 148a | 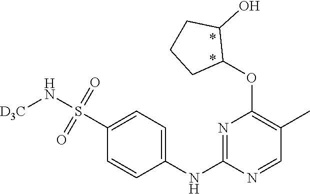 [SMILES: [2H]C([2H])([2H])NS(=O)(=O)c1ccc(Nc2ncc(C)c(OC3CCCC3O)n2)cc1] Enantiomer 1 (earlier eluting enantiomer), from cis-cyclopentane 1,2-diol | LC-MS (ESI): m/z 382.2 [M + H]⁺; ¹H NMR (400 MHz, DMSO-d₆) δ 9.75 (s, 1H), 8.09 (s, 1H), 7.93 (d, J = 8.8 Hz, 2H), 7.65 (d, J = 8.8 Hz, 2H), 7.16 (s, 1H), 5.28-5.20 (m, 1H), 4.62 (d, J = 5.0 Hz, 1H), 4.26-4.14 (m, 1H), 2.06- 1.97 (m, 1H), 2.03 (s, 3H), 1.86- 1.75 (m, 3H), 1.71-1.62 (m, 1H), 1.59-1.48 (m, 1H). ee: 93.4% Retention time: 9.55 min; Column: ChiralPAK IC, 250 × 21.2 mm I.D., 5 μm; Mobile phase: A for CO₂ and B for methanol (0.05% DEA); |
| | | Gradient: 10 min @ 40%; Flow |
| | | rate: 40 mL/min; Column |
| | | temperature: 35° C. |
| 148b | 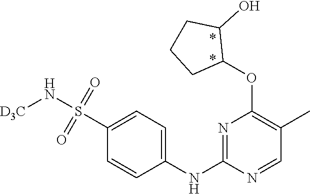 [SMILES: [2H]C([2H])([2H])NS(=O)(=O)c1ccc(Nc2ncc(C)c(OC3CCCC3O)n2)cc1] Enantiomer 2 (later eluting enantiomer), from cis-cyclopentane 1,2-diol | LC-MS (ESI): m/z 382.2 [M + H]⁺; ¹H NMR (400 MHz, DMSO-d₆) δ 9.75 (s, 1H), 8.09 (s, 1H), 7.93 (d, J = 8.8 Hz, 2H), 7.65 (d, J = 8.8 Hz, 2H), 7.16 (s, 1H), 5.30-5.18 (m, 1H), 4.62 (d, J = 5.0 Hz, 1H), 4.27-4.13 (m, 1H), 2.08- 1.99 (m, 1H), 2.03 (s, 3H), 1.89- 1.76 (m, 3H), 1.70-1.61 (m, 1H), 1.58-1.49 (m, 1H). ee: 93.4% Retention time: 9.55 min; Column: ChiralPAK IC, 250 × 21.2 mm I.D., 5 μm; Mobile phase: A for CO₂ and B for methanol (0.05% DEA); |
| | | Gradient: 10 min @ 40%; Flow |
| | | rate: 40 mL/min; Column |
| | | temperature: 35° C. |
| 149a | 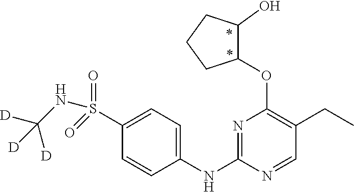 [SMILES: [2H]C([2H])([2H])NS(=O)(=O)c1ccc(Nc2ncc(CC)c(OC3CCCC3O)n2)cc1] Enantiomer 1 (earlier eluting enantiomer), from cis-cyclopentane 1,2-diol | LC-MS (ESI): m/z 396.2 [M + H]⁺; ¹H NMR (400 MHz, DMSO-d₆) δ 9.76 (s, 1H), 8.08 (s, 1H), 7.93 (d, J = 8.8 Hz, 2H), 7.65 (d, J = 8.8 Hz, 2H), 7.16 (s, 1H), 5.26 (d, J = 5.0 Hz, 1H), 4.65 (d, J = 5.0 Hz, 1H), 4.25-4.16 (m, 1H), 2.49-2.42 (m, 2H), 2.05-1.97 (m, 1H), 1.91-1.73 (m, 3H), 1.71- 1.52 (m, 2H), 1.14 (t, J = 7.6 Hz, 3H). ee: 94.7% Retention time: 4.41 min; Column: ChiralPak IA, 250 × 4.6 mm I.D., 5 μm, Mobile phase: A for CO₂ and B for MeOH (0.05% |
| | | DEA), Gradient: 8 min @ B 40%, |
| | | Flow rate: 2.0 mL/min, Back |
| | | pressure: 100 bar, Column |
| | | temperature: 35° C. |
| 149b | 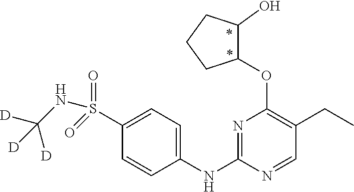 [SMILES: [2H]C([2H])([2H])NS(=O)(=O)c1ccc(Nc2ncc(CC)c(OC3CCCC3O)n2)cc1] Enantiomer 2 (later eluting enantiomer), from cis-cyclopentane 1,2-diol | LC-MS (ESI): m/z 396.2 [M + H]⁺. ee: 93.3% Retention time: 5.78 min; Column: ChiralPak IA, 250 × 4.6 mm I.D., 5 μm, Mobile phase: A for CO₂ and B for MeOH (0.05% DEA), Gradient: 8 min @ B 40%, Flow rate: 2.0 mL/min, Back pressure: 100 bar, Column temperature: 35° C. |
| 150a | 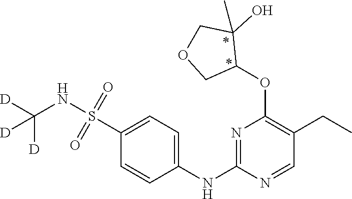 [SMILES: [2H]C([2H])([2H])NS(=O)(=O)c1ccc(Nc2ncc(CC)c(OC3COCC3(C)O)n2)cc1] Enantiomer 1 (earlier eluting enantiomer), from 3- methyltetrahydrofuran-3,4-diol | LC-MS (ESI): m/z 412.2 [M + H]⁺; ¹H NMR (400 MHz, DMSO-d₆) δ 9.78 (s, 1H), 8.12 (s, 1H), 7.91 (d, J = 8.8 Hz, 2H), 7.66 (d, J = 8.8 Hz, 2H), 7.18 (s, 1H), 5.27-5.18 (m, 1H), 4.95 (s, 1H), 4.30-4.24 (m, 1H), 3.77-3.75 (m, 1H), 3.66 (d, J = 8.2 Hz, 1H), 3.57 (d, J = 8.2 Hz, 1H), 2.58-2.49 (m, 2H), 1.35 (s, 3H), 1.14 (t, J = 7.4 Hz, 3H). ee: 98.7% Retention time: 5.11 min; Column: ChiralPak AD, 250 × 4.6 mm I.D., 5 μm, Mobile phase: A for CO₂ and B for methanol |
| | | (0.05% DEA), Gradient: 8 min |
| | | @ B 40%, Flow rate: 2.0 mL/min, |
| | | Back pressure: 100 bar, Column |
| | | temperature: 35° C. |
| 150b | 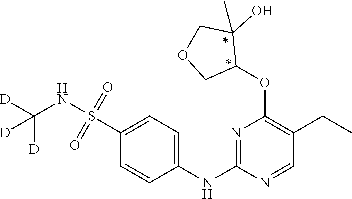 [SMILES: [2H]C([2H])([2H])NS(=O)(=O)c1ccc(Nc2ncc(CC)c(OC3COCC3(C)O)n2)cc1] Enantiomer 2 (later eluting enantiomer), from 3- methyltetrahydrofuran-3,4-diol | LC-MS (ESI): m/z 412.2 [M + H]⁺. ee: 97.9% Retention time: 9.10 min; Column: ChiralPak AD, 250 × 4.6 mm I.D., 5 μm, Mobile phase: A for CO₂ and B for methanol (0.05% DEA), Gradient: 8 min @ B 40%, Flow rate: 2.0 mL/min, Back pressure: 100 bar, Column temperature: 35° C. |
| 151a | 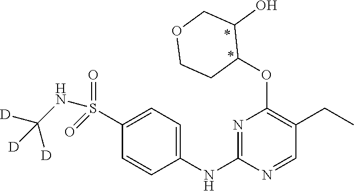 [SMILES: [2H]C([2H])([2H])NS(=O)(=O)c1ccc(Nc2ncc(CC)c(OC3CCOCC3O)n2)cc1] Enantiomer 1 (earlier eluting enantiomer), made from cis- tetrahydro-2H-pyran-3,4-diol | LC-MS (ESI): m/z 412.2 [M + H ]⁺; ¹H NMR (400 MHz, DMSO-d₆) δ 9.81 (s, 1H), 8.12 (s, 1H), 7.92 (d, J = 8.8 Hz, 2H), 7.66 (d, J = 8.8 Hz, 2H), 7.17 (s, 1H), 5.48-5.39 (m, 1H), 5.02-4.91 (m, 1H), 3.92-3.80 (m, 1H), 3.66-3.59 (m, 4H), 3.33-3.38 (m, 2H), 2.07- 2.03 (m, 1H), 1.90-1.82 (m, 1H), 1.16 (t, J = 7.2 Hz, 3H). ee: 100% Retention time: 2.30 min; Column: Chiralpak IC-3, 150 × 4.6 mm I.D., 3 μm, Mobile phase: 40% of ethanol (0.05% DEA) in CO₂, Flow rate: 2.5 mL/min, |
| | | Column temperature: 35° C. |
| 151b | 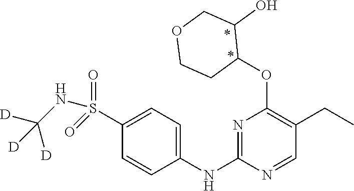 [SMILES: [2H]C([2H])([2H])NS(=O)(=O)c1ccc(Nc2ncc(CC)c(OC3CCOCC3O)n2)cc1] Enantiomer 2 (later eluting enantiomer), made from cis- tetrahydro-2H-pyran-3,4-diol | LC-MS (EIS): m/z 412.2 [M + H]⁺. ee: 99.3% Retention time: 3.07 min; Column: Chiralpak IC-3, 150 × 4.6 mm I.D., 3 μm, Mobile phase: 40% of ethanol (0.05% DEA) in CO₂, Flow rate: 2.5 mL/min, Column temperature 35° C. |
| 152a | 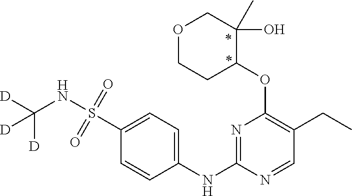 [SMILES: [2H]C([2H])([2H])NS(=O)(=O)c1ccc(Nc2ncc(CC)c(OC3CCOCC3(C)O)n2)cc1] Enantiomer 1 (earlier eluting enantiomer), made from cis-3- methyltetrahydro-2H-pyran-3,4- diol | LC-MS (ESI): m/z 426.2 [M + H]⁺; ¹H NMR (400 MHz, DMSO-d₆) δ 9.79 (s, 1H), 8.11 (s, 1H), 7.91 (d, J = 8.8 Hz, 2H), 7.66 (d, J = 8.8 Hz, 2H), 7.18 (s, 1H), 5.14 (t, J = 6.0 Hz, 1H), 4.66 (s, 1H), 3.80-3.77 (m, 1H), 3.58-3.53 (m, 3H), 3.32-3.34 (m, 2H), 1.97- 1.91 (m, 2H), 1.15 (t, J = 7.4 Hz, 3H), 1.14 (s, 3H). ee: 99.5% Retention time: 4.58 min; Column: ChiralPak AD, 250 × 4.6 mm I.D., 5 μm, Mobile phase: A for CO₂ and B for methanol (0.05% DEA); Gradient: 0.0 min- 1.0 min @ 10% B, 1.0 min-4.5 |
| | | min gradient (10-40% B), 4.5 |
| | | min-7.0 min @ 40% B, 7.0 min- |
| | | 8.0 min @ 10% B; Flow rate: 2.5 |
| | | mL/min; Column temperature: |
| | | 40° C. |
| 152b | 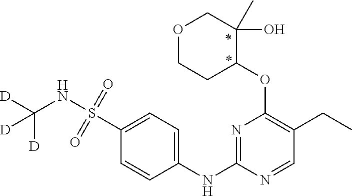 [SMILES: [2H]C([2H])([2H])NS(=O)(=O)c1ccc(Nc2ncc(CC)c(OC3CCOCC3(C)O)n2)cc1] Enantiomer 2 (later eluting enantiomer), made from cis-3- methyltetrahydro-2H-pyran-3,4- diol | LC-MS (ESI): m/z 426.2 [M + H]⁺. ee: 94.1% Retention time: 5.26 min; Column: ChiralPak AD, 250 × 4.6 mm I.D., 5 μm; Mobile phase: A for CO₂ and B for methanol (0.05% DEA); Gradient: 0.0 min- 1.0 min @ 10% B, 1.0 min-4.5 min gradient (10-40% B), 4.5 min-7.0 min @ 40% B, 7.0 min- 8.0 min @ 10% B; Flow rate: 2.5 mL/min; Column temperature: 40° C. |
| 153a | 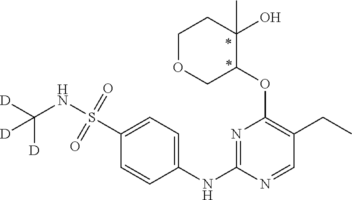 [SMILES: [2H]C([2H])([2H])NS(=O)(=O)c1ccc(Nc2ncc(CC)c(OC3COCCC3(C)O)n2)cc1] Enantiomer 1 (earlier eluting enantiomer), made form cis-4- methyltetrahydro-2H-pyran-3,4- diol | LC-MS (ESI): m/z 426.2 [M + H]⁺; ¹H NMR (400 MHz, DMSO-d₆) δ 9.83 (s, 1H), 8.12 (s, 1H), 7.93 (d, J = 8.8 Hz, 2H), 7.64 (d, J = 8.8 Hz, 2H), 7.18 (s, 1H), 5.05-4.95 (m, 1H), 4.65 (s, 1H), 3.83-3.77 (m, 1H), 3.75-3.67 (m, 1H), 3.66-3.55 (m, 2H), 3.32-3.24 (m, 2H), 1.75-1.68 (m, 2H), 1.22 (s, 3H), 1.13 (t, J = 7.4 Hz, 3H). ee: 100% Retention time: 1.76 min; Column: Chiralpak AD-3, 150 × 4.6 mm I.D., 3 μm Mobile phase: 40% of isopropanol (0.05% DEA) in CO₂, Flow rate: 2.5 mL/min, Column temperature: 35° C. |
| 153b | 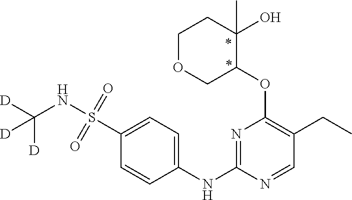 [SMILES: [2H]C([2H])([2H])NS(=O)(=O)c1ccc(Nc2ncc(CC)c(OC3COCCC3(C)O)n2)cc1] Enantiomer 2 (later eluting enantiomer), made from cis-4- methyltetrahydro-2H-pyran-3,4- diol | LC-MS (ESI): m/z 426.2 [M + H]⁺. ee: 99.1% Retention time: 2.15 min; Column: Chiralpak AD-3, 1.50 × 4.6 mm I.D., 3 μm Mobile phase: 40% of isopropanol (0.05% DEA) in CO₂, Flow rate: 2.5 mL/min, Column temperature: 35° C. |
| 154a | 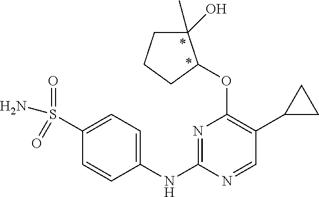 [SMILES: CC1(O)CCCC1Oc1nc(Nc2ccc(S(N)(=O)=O)cc2)ncc1C1CC1] Enantiomer 1 (earlier eluting enantiomer), made from cis-1- methylcyclopentane-1,2-diol | LC-MS (ESI): m/z 405.2 [M + H]⁺; ¹H NMR (400 MHz, DMSO-d₆) δ 9.70 (s, 1H), 7.93 (s, 1H), 7.89 (d, J = 8.8 Hz, 2H), 7.70 (d, J = 8.8 Hz, 2H), 7.14 (s, 2H), 5.03 (t, J = 6.4 Hz, 1H), 4.37 (s, 1H), 2.24-2.13 (m, 1H), 2.03-1.90 (m, 1H), 1.87-1.70 (m, 3H), 1.66- 1.56 (m, 2H), 1.26 (s, 3H), 0.87- 0.62 (m, 4H). ee: 98.6% Retention time: 3.99 min; Column: ChiralPak AD, 250 × 4.6 mm I.D., 5 μm, Mobile phase: A for CO₂ and B for MeOH (0.05% DEA), Gradient: 8 min @ B 30%, |
| | | Flow rate: 2.0 mL/min, Back |
| | | pressure: 100 bar, Column |
| | | temperature: 35° C. |
| 154b | 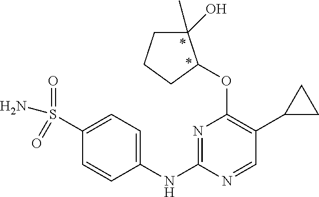 [SMILES: CC1(O)CCCC1Oc1nc(Nc2ccc(S(N)(=O)=O)cc2)ncc1C1CC1] Enantiomer 2 (later eluting enantiomer), made from cis-1- methylcyclopentane-1,2-diol | LC-MS (ESI): m/z 405.2 [M + H]⁺. ee; 97.1% Retention time: 5.65 min; Column: ChiralPak AD, 250 × 4.6 mm I.D., 5 μm, Mobile phase: A for CO₂ and B for MeOH (0.05% DEA), Gradient: 8 min @ B 30%, Flow rate: 2.0 mL/min, back pressure: 100 bar, Column temperature: 35° C. |
| 155a | 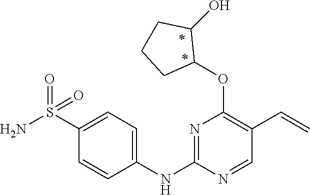 [SMILES: C=Cc1cnc(Nc2ccc(S(N)(=O)=O)cc2)nc1OC1CCCC1O] Enantiomer 1 (earlier eluting enantiomer), from cis-cyclopentane 1,2-diol | LC-MS (ESI): m/a 377.1 [M + H]⁺; ¹H NMR (400 MHz, DMSO-d₆) δ 9.97 (s, 1H), 8.43 (s, 1H), 7.92 (d, J = 8.8 Hz, 2H), 7.74 (d, J = 9.2 Hz, 2H), 7.17 (s, 2H), 6.71-6.63 (m, 1H), 5.95-5.90 (m, 1H), 5.30-5.20 (m, 2H), 4.76 (d, J = 4.8 Hz, 1H), 4.25 (t, J = 4.8 Hz, 1H), 2.08-2.02 (m, 1H), 1.90-1.75 (m, 3H), 1.70-1.54 (m, 2H). ee: 95.3% Retention time: 5.53 min; Column: ChiralPak IA, 250 × 4.6 mm I.D., 5 μm, Mobile phase: A for CO₂ and B for MeOH (0.05% DEA), Gradient: 8 min @ B 40%, |
| | | Flow rate: 1.8 mL/min, Back |
| | | pressure: 100 bar, Column |
| | | temperature: 35° C. |
| 155b | 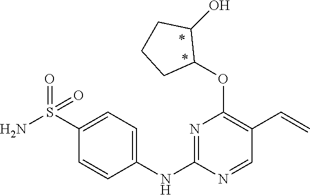 [SMILES: C=Cc1cnc(Nc2ccc(S(N)(=O)=O)cc2)nc1OC1CCCC1O] Enantiomer 2 (later eluting enantiomer), from cis-cyclopentane 1,2-diol | LC-MS (ESI): m/z 377.1 [M + H]⁺. ee: 92.3% Retention time: 6.55 min; Column: ChiralPak IA, 250 × 4.6 mm I.D., 5 μm, Mobile phase: A for CO₂ and B for MeOH (0.05% DEA), Gradient: 8 min @ B 40%, Flow rate: 1.8 mL/min, Back pressure: 100 bar, Column temperature: 35° C. |

### Biological Example 1. Measurement of Kinase Inhibitory Activity

CDK2/CyclinE1 kinase inhibitory activity (IC50): 5 μl of various dilutions of test compounds in 1×kinase buffer (50 mM HEPES pH 7.5, 10 mM MgCl2, 2 mM DTT and 0.01% Brij-35) were mixed with 10 μL of CDK2/CyclinE1 (Carna, 04-165 #, final concentration 3 nM in 1×Kinase buffer) in 384 plates and incubated at room temperature for 10 min. To initiate each reaction, 10 μL of peptide solution containing fluorescently-labeled-peptide 18(5-FAM-QSPKKG-CONH2) (GL, 114202 #, final concentration 3000 nM) and ATP (final concentration 77 M) in 1×Kinase buffer was added to each of the wells containing test compound and CDK2/CyclinE1 mixture. The reaction was then allowed to proceed at 28° C. for 30 min and terminated by the addition of 25 μL stop buffer (100 mM HEPES pH 7.5, 50 mM EDTA, 0.2% Coating Reagent #3 (Perkin Elmer, 760050 #) and 0.015% Brij-35).

Following the kinase reaction, Caliper EZ reader II (Downstream voltages: −500V, Upstream voltages: −2250V, Base pressure −0.5 PSI, Screen pressure −1.2 PSI) was used to separate the phosphorylated (product) and the unphosphorylated (substrate) fluorescently-labeled peptide 18 based on their different mobility. Both substrate and product were measured and the ratio of these values were used to generate % conversion by Caliper EZ reader II. These conversion values were then transformed into % inhibition of kinase activity using the formula: % Inhibition=[(MA−X)/(MA−MI)]×100% where MA=conversion value of DMSO only controls, MI=conversion value of no enzyme controls and X=conversion value at any given compound dose. IC50 values were then calculated by plotting dose-response curves and then using the XLfit application in Excel software.

CDK1/CyclinB kinase inhibitory activity (IC50): 5 μl of various dilutions of test compound in 1×kinase buffer (50 mM HEPES pH 7.5, 10 mM MgCl2, 2 mM DTT and 0.01% Brij-35) was mixed with 10 μL of CDK1/CyclinB (Millipore, 14-450M #, final concentration 3 nM in 1×Kinase buffer) in 384 plates and incubated at room temperature for 10 min. To initiate each reaction, 10 μL of peptide solution containing fluorescently-labeled −peptide 18(5-FAM-QSPKKG-CONH2) (GL, 114202 #, final concentration 3000 nM) and ATP (final concentration 20 M) in 1×Kinase buffer was added to each of the wells containing test compound and CDK1/CyclinB mixture. The reaction is then allowed to proceed at 28° C. for 30 min and terminated by the addition of 25 μL stop buffer (100 mM HEPES pH 7.5, 50 mM EDTA, 0.2% Coating Reagent #3 (Perkin Elmer, 760050 #) and 0.015% Brij-35).

Following the kinase reaction, Caliper EZ reader II (Downstream voltages: −500V, Upstream voltages: −2250V, Base pressure −0.5 PSI, Screen pressure −1.2 PSI) was used to separate the phosphorylated (product) and the unphosphorylated (substrate) fluorescently-labeled peptide 18 based on their different mobility. Both substrate and product were measured and the ratio of these values were used to generate % conversion by Caliper EZ reader II. These conversion values were then transformed into % inhibition of kinase activity using the formula: % Inhibition=[(MA−X)/(MA−MI)]×100% where MA=conversion value of DMSO only controls, MI=conversion value of no enzyme controls and X=conversion value at any given compound dose. IC50 values were then calculated by plotting dose-response curves and then using the XLfit application in Excel software.

CDK4/CyclinD1 kinase inhibitory activity (IC50): 5 l of various dilutions of test compound in 1×kinase buffer (20 mM HEPES, pH 7.5, 10 mM MgCl2, 2 mM DTT and 0.01% Triton X-100) was mixed with 10 μL of either CDK4/Cyclin D1 (ProQinase, 0142-0143-1 #, final concentration 20 nM in 1×Kinase buffer) or CDK4/CyclinD3 (Carna, 04-105 #, final concentration 10 nM in 1×Kinase buffer) in 384 plates and incubated at room temperature for 10 min. To initiate each reaction, 10 μL of peptide solution containing fluorescently-labeled-peptide 8(5-FAM-IPTSPITTTYFFFKKK-COOH, GL, 112396 #, final concentration 3000 nM) and ATP (final concentration 672 uM for CDK4/CyclinD1 or 280 M for CDK4/Cyclin D3) in 1×Kinase buffer was added to each of the wells containing test compound and CDK4/CyclinD3 mixture. The reaction is then allowed to proceed at 28° C. for 30 min and terminated by the addition of 25 μL stop buffer (100 mM HEPES pH 7.5, 50 mM EDTA, 0.2% Coating Reagent #3 (Perkin Elmer, 760050 #) and 0.015% Brij-35).

Following the kinase reaction, Caliper EZ reader II (Downstream voltages: −500V, Upstream voltages: −2250V, Base pressure −0.5 PSI, Screen pressure −1.2 PSI) was used to separate the phosphorylated (product) and the unphosphorylated (substrate) fluorescently-labeled peptide 8 based on their different mobility. Both substrate and product were measured and the ratio of these values were used to generate % conversion by Caliper EZ reader II. These conversion values were then transformed into % inhibition of kinase activity using the formula: % Inhibition=[(MA−X)/(MA−MI)]×100% where MA=conversion value of DMSO only controls, MI=conversion value of no enzyme controls and X=conversion value at any given compound dose. IC50 values were then calculated by plotting dose-response curves and then using the XLfit application in Excel software.

CDK6/CyclinD1 kinase inhibitory activity (IC50): 5 l of various dilutions of test compound in 1×kinase buffer (50 mM HEPES pH 7.5, 10 mM MgCl2, 2 mM DTT and 0.01% Brij-35) was mixed with 10 μL of CDK6/CyclinD1 (Carna, 04-114 #, final concentration 7.5 nM in 1×Kinase buffer) or CDK6/Cyclin D3 (Carna, 04-107 #, final concentration 15 nM in 1×Kinase buffer) in 384 plates and incubated at room temperature for 10 min. To initiate each reaction, 10 μL of peptide solution containing fluorescently-labeled-peptide 8(5-FAM-IPTSPITTTYFFFKKK-COOH, GL, 112396 #, final concentration 3000 nM) and ATP (final concentration 230 M for CDK6/CyclinD1 or 800 uM for CDK6/CyclinD3) in 1×Kinase buffer was added to each of the wells containing test compound and CDK6/CyclinD1 or CDK6/Cyclin D3 mixture. The reaction is then allowed to proceed at 28° C. for 30 min and terminated by the addition of 25 μL stop buffer (100 mM HEPES pH 7.5, 50 mM EDTA, 0.2% Coating Reagent #3 (Perkin Elmer, 760050 #) and 0.015% Brij-35).

Following the kinase reaction, Caliper EZ reader II (Downstream voltages: −500V, Upstream voltages: −2250V, Base pressure −0.5 PSI, Screen pressure −1.2 PSI) was used to separate the phosphorylated (product) and the unphosphorylated (substrate) fluorescently-labeled peptide 8 based on their different mobility. Both substrate and product were measured and the ratio of these values were used to generate % conversion by Caliper EZ reader II. These conversion values were then transformed into % inhibition of kinase activity using the formula: % Inhibition=[(MA−X)/(MA−MI)]×100% where MA=conversion value of DMSO only controls, MI=conversion value of no enzyme controls and X=conversion value at any given compound dose. IC50 values were then calculated by plotting dose-response curves and then using the XLfit application in Excel software.

CDK7/CyclinHIMAT1 kinase inhibitory activity (IC50): 5 l of various dilutions of test compound in 1×kinase buffer (20 mM HEPES, pH 7.5, 10 mM MgCl2, 2 mM DTT and 0.01% Triton X-100) was mixed with 10 μL of CDK7/CyclinH/MAT1 (Millipore, 14-476M #, final concentration 12.5 nM in 1×Kinase buffer) in 384 plates and incubated at room temperature for 10 min. To initiate each reaction, 10 μL of peptide solution containing fluorescently-labeled-peptide CTD3 (5-FAM-ACSYSPTSPSYSPTSPSYSPTSPSKK, GL, SY346885 #, final concentration 3000 nM) and ATP (final concentration 70 M) in 1×Kinase buffer was added to each of the wells containing test compound and CDK7/CyclinH/MAT1 mixture. The reaction is then allowed to proceed at 28° C. for 30 min and terminated by the addition of 25 μL stop buffer (100 mM HEPES pH 7.5, 50 mM EDTA, 0.2% Coating Reagent #3 (Perkin Elmer, 760050 #) and 0.015% Brij-35).

Following the kinase reaction, Caliper EZ reader II (Downstream voltages: −500V, Upstream voltages: −2250V, Base pressure −0.5 PSI, Screen pressure −1.2 PSI) was used to separate the phosphorylated (product) and the unphosphorylated (substrate) fluorescently-labeled peptide CTD3 based on their different mobility. Both substrate and product were measured and the ratio of these values were used to generate % conversion by Caliper EZ reader II. These conversion values were then transformed into % inhibition of kinase activity using the formula: % Inhibition=[(MA−X)/(MA−MI)]×100% where MA=conversion value of DMSO only controls, MI=conversion value of no enzyme controls and X=conversion value at any given compound dose. IC50 values were then calculated by plotting dose-response curves and then using the XLfit application in Excel software.

CDK9/CyclinT1 kinase inhibitory activity (IC50): 5 l of various dilutions of test compound in 1×kinase buffer (20 mM HEPES, pH 7.5, 10 mM MgCl2, 2 mM DTT and 0.01% Triton X-100) was mixed with 10 μL of CDK9/CyclinT1 (Millipore, 14-685M #, final concentration 12.5 nM in 1×Kinase buffer) in 384 plates and incubated at room temperature for 10 min. To initiate each reaction, 10 μL of peptide solution containing fluorescently-labeled-peptide CTD3 (5-FAM-ACSYSPTSPSYSPTSPSYSPTSPSKK, GL, SY346885 #, final concentration 3000 nM) and ATP (final concentration 10 μM) in 1×Kinase buffer was added to each of the wells containing test compound and CDK9/CyclinT1 mixture. The reaction is then allowed to proceed at 28° C. for 30 min and terminated by the addition of 25 μL stop buffer (100 mM HEPES pH 7.5, 50 mM EDTA, 0.2% Coating Reagent #3 (Perkin Elmer, 760050 #) and 0.015% Brij-35).

Following the kinase reaction, Caliper EZ reader II (Downstream voltages: −500V, Upstream voltages: −2250V, Base pressure −0.5 PSI, Screen pressure −1.2 PSI) was used to separate the phosphorylated (product) and the unphosphorylated (substrate) fluorescently-labeled peptide CTD3 based on their different mobility. Both substrate and product were measured and the ratio of these values were used to generate % conversion by Caliper EZ reader II. These conversion values were then transformed into % inhibition of kinase activity using the formula: % Inhibition=[(MA−X)/(MA−MI)]×100% where MA=conversion value of DMSO only controls, MI=conversion value of no enzyme controls and X=conversion value at any given compound dose. IC50 values were then calculated by plotting dose-response curves and then using the XLfit application in Excel software.

Biological activity data for representative compounds of the present disclosure are provided in Table 2 below. Exemplary results are presented as calculated IC₅₀ values. In Table 2, “A” represents a calculated IC₅₀ value of less than 10 nM; “B” represents a calculated IC₅₀ value of greater than or equal to 10 nM and less than 100 nM; “C” represents a calculated IC₅₀ value of greater than or equal to 100 nM and less than 1 μM; and “D” represents a calculated IC₅₀ value of 1 μM or greater.

TABLE 2
Selected in vitro data on different CDKs

| | | | CDK4/Cyclin | CDK6/Cyclin | CDK7/CyclinH | CDK9/Cyclin |
|---|---|---|---|---|---|---|
| Example | CDK1/Cyclin | CDK2/Cyclin | D1 IC50 | D1 IC50 | /MAT1 IC50 | T1 IC50 |
| number | B1 IC50(nM) | El IC50 (nM) | (nM) | (nM) | (nM) | (nM) |
| 1 | B | B | | D | D | B |
| 2a | B | A | B | B | D | C |
| 2b | C | B | | | | D |
| 3a | C | B | | | | D |
| 3b | B | A | A | A | B | C |
| 4a | B | A | B | A | B | C |
| 4b | C | B | C | B | | D |
| 5a | B | A | A | B | | C |
| 5b | C | B | C | B | | D |
| 6a | B | A | A | B | C | D |
| 6b | C | B | | | | C |
| 6c | C | B | | | | D |
| 6d | D | D | | | | D |
| 7a | A | A | A | A | B | C |
| 7b | | B | C | C | D | |
| 8 | A | A | A | B | C | C |
| 9 | B | A | B | B | D | C |
| 10 | B | A | B | B | C | B |
| 11 | A | A | | | | B |
| 12 | B | A | A | C | D | B |
| 13 | B | B | | | | |
| 14 | C | B | | D | D | C |
| 15 | B | A | | C | C | C |
| 16 | C | B | | | | |
| 17 | B | A | | C | D | C |
| 18a | C | B | | | | C |
| 18b | B | A | | | | C |
| 19a | B | A | | | | C |
| 19b | B | A | | | | C |
| 20a | C | A | C | C | D | C |
| 20b | C | B | C | C | D | C |
| 21a | C | A | | | | C |
| 21b | C | B | | | | C |
| 22a | B | A | B | B | D | C |
| 22b | B | A | | | | C |
| 23a | A | A | B | B | C | B |
| 23b | C | B | | | | C |
| 24a | A | A | A | A | A | A |
| 24b | B | B | | | | C |
| 25 | B | A | C | B | D | C |
| 26 | C | A | C | C | D | D |
| 27 | B | A | C | C | D | C |
| 28 | B | A | | | | C |
| 29a | C | A | | | | C |
| 29b | B | A | | | | C |
| 30a | B | A | | | | C |
| 30b | C | B | | | | D |
| 31a | B | A | B | C | D | C |
| 31b | C | B | | | | D |
| 32a | B | A | C | C | D | C |
| 32b | C | B | | | | D |
| 33a | B | A | B | C | D | C |
| 33b | C | A | | | | D |
| 34a | A | A | B | B | B | C |
| 34b | C | A | | | | D |
| 35a | A | A | B | B | B | B |
| 35b | B | A | | | | C |
| 36a | C | B | | | | D |
| 36b | B | A | A | B | C | C |
| 37a | B | A | A | B | B | C |
| 37b | C | B | | | | D |
| 38a | B | A | A | A | B | C |
| 38b | C | B | | | | D |
| 39a | B | A | B | A | C | C |
| 39b | C | B | | | | D |
| 40a | B | A | A | A | C | C |
| 40b | | B | B | B | D | |
| 41a | B | A | A | A | C | C |
| 41b | C | B | B | B | | D |
| 42a | | A | B | B | C | |
| 42b | | B | C | C | D | |
| 43a | B | A | A | B | C | C |
| 43b | | B | B | B | D | |
| 44a | C | B | C | B | | D |
| 44b | B | A | B | A | B | C |
| 45a | B | A | B | B | B | C |
| 45b | D | C | C | C | | D |
| 46a | | C | C | C | | |
| 46b | A | A | A | A | A | B |
| 47a | B | A | A | A | B | C |
| 47b | D | C | C | C | D | D |
| 48a | A | A | A | A | B | C |
| 48b | B | A | B | B | | D |
| 49a | A | A | A | A | B | D |
| 49b | | B | C | B | C | |
| 49c | B | A | B | B | D | |
| 49d | | C | D | C | D | |
| 50a | A | A | A | A | C | C |
| 50b | | A | B | B | D | |
| 51a | | A | B | B | C | |
| 51b | | B | C | C | D | |
| 52 | B | A | A | A | C | C |
| 53 | B | A | A | B | C | C |
| 54 | B | A | A | A | C | C |
| 55 | B | A | A | B | D | C |
| 56 | A | A | A | B | C | C |
| 57 | A | A | A | A | C | B |
| 58 | B | A | A | B | C | C |
| 59 | B | A | A | B | C | C |
| 60 | A | A | A | B | C | C |
| 61 | B | A | A | B | C | C |
| 62 | A | A | A | B | C | C |
| 63 | A | A | A | A | C | C |
| 64a | | A | B | B | C | |
| 64b | B | A | B | A | C | C |
| 65 | B | A | B | C | D | C |
| 66 | B | A | | C | D | C |
| 67 | C | B | | | D | C |
| 68 | B | A | B | B | D | B |
| 69 | B | A | | | C | C |
| 70 | C | B | | | D | C |
| 71 | B | A | | C | D | B |
| 72 | C | B | | C | D | C |
| 73 | B | A | | C | C | C |
| 74 | B | A | | C | D | C |
| 75 | B | A | | | | B |
| 76 | C | B | | | | C |
| 77 | B | A | | | | B |
| 78 | B | A | | | | C |
| 79 | C | B | | | | C |
| 80 | C | B | | | | |
| 81 | C | B | B | C | D | C |
| 82 | C | B | C | C | D | C |
| 83 | B | A | | C | D | B |
| 84 | B | B | | | | |
| 85 | B | A | | C | C | C |
| 86 | B | B | | D | D | B |
| 87 | B | A | | | | |
| 88 | B | A | | | | |
| 89 | C | B | | | D | D |
| 90 | C | B | | | | |
| 91 | B | A | B | C | D | C |
| 92 | B | A | | C | D | C |
| 93 | C | B | | D | D | C |
| 94 | B | A | B | C | D | B |
| 95 | B | B | C | D | D | |
| 96 | C | B | B | D | D | C |
| 97 | C | B | C | D | D | |
| 98 | B | A | B | B | D | B |
| 99a | B | A | C | B | C | B |
| 99b | B | A | | | | C |
| 100a | B | A | C | C | C | C |
| 100b | C | B | | | | C |
| 101a | C | A | D | D | D | D |
| 101b | | B | D | D | | |
| 102a | C | A | D | D | D | |
| 102b | B | A | D | C | C | |
| 102c | C | A | D | C | D | |
| 102d | C | A | D | C | D | |
| 103a | B | A | C | C | C | C |
| 103b | C | A | D | C | D | |
| 104a | B | A | B | B | B | C |
| 104b | C | B | C | C | | D |
| 105a | B | A | C | C | C | C |
| 105b | B | A | | | | C |
| 106a | A | A | B | C | C | B |
| 106b | B | A | | | | C |
| 107a | B | A | C | C | C | C |
| 107b | B | A | | | | C |
| 108 | B | A | B | C | D | D |
| 109a | B | A | C | C | C | C |
| 109b | C | B | | | | C |
| 110a | B | A | C | B | C | C |
| 110b | C | A | | | | C |
| 111a | C | A | | | | C |
| 111b | A | A | B | B | C | B |
| 112a | B | A | C | C | D | C |
| 112b | C | A | | | | C |
| 113a | C | A | | | | C |
| 113b | B | A | | | | C |
| 114a | B | A | C | C | C | B |
| 114b | C | B | | | | C |
| 115a | B | A | | | B | |
| 115b | C | B | | | | C |
| 116a | B | A | C | C | C | C |
| 116b | C | B | | | | D |
| 117a | B | A | C | C | C | C |
| 117b | C | B | | | | D |
| 118a | A | A | B | C | C | A |
| 118b | B | A | | | | B |
| 119a | B | A | B | B | B | C |
| 119b | C | B | C | B | C | C |
| 120a | B | A | B | B | B | C |
| 120b | C | B | | | | D |
| 121a | B | A | B | B | B | C |
| 121b | C | B | | | | C |
| 122a | C | A | | | | C |
| 122b | B | A | B | B | B | B |
| 123a | C | B | | | | C |
| 123b | B | A | B | B | C | C |
| 124a | B | A | B | B | A | B |
| 124b | D | C | C | C | | D |
| 125a | A | A | A | A | A | B |
| 125b | | B | C | C | C | |
| 126 | B | A | B | B | C | C |
| 127 | B | A | A | C | C | B |
| 128 | B | A | A | B | D | B |
| 129 | B | A | A | C | D | B |
| 130a | B | A | C | C | C | C |
| 130b | B | B | | | | C |
| 131a | B | A | C | C | C | C |
| 131b | C | A | | | | C |
| 132a | C | A | C | C | C | C |
| 132b | C | B | | | | C |
| 133a | B | A | C | B | C | B |
| 133b | C | A | | | | C |
| 134a | B | A | C | C | C | C |
| 134b | C | B | | | | C |
| 135a | B | A | C | C | C | C |
| 135b | C | B | | | | C |
| 136a | B | A | C | B | C | B |
| 136b | B | A | | | | C |
| 137a | B | A | C | B | C | C |
| 137b | B | A | | | | C |
| 138a | B | A | C | C | C | C |
| 138b | B | A | | | | C |
| 139a | B | A | C | B | C | C |
| 139b | C | B | C | C | D | D |
| 140a | B | A | C | C | C | C |
| 140b | D | B | | | | D |
| 141a | B | A | B | B | C | B |
| 141b | B | A | | | | C |
| 142a | B | A | C | B | C | C |
| 142b | B | A | | | | C |
| 143a | B | A | C | B | C | C |
| 143b | C | A | | | | C |
| 144a | C | B | | | | D |
| 144b | B | A | B | B | C | C |
| 145a | C | B | | | | D |
| 145b | B | A | B | B | B | C |
| 146a | C | A | D | C | D | C |
| 146b | C | B | | | | D |
| 147a | B | A | D | C | C | C |
| 147b | C | B | | | | C |
| 148a | C | A | D | C | D | C |
| 148b | C | B | | | | D |
| 149a | B | A | C | C | C | C |
| 149b | C | B | | | | C |
| 150a | B | A | C | C | C | C |
| 150b | D | C | | | | |
| 151a | C | A | D | C | C | D |
| 151b | D | B | | | | D |
| 152a | A | A | | | | B |
| 152b | D | B | | | | D |
| 153a | B | A | C | B | B | B |
| 153b | D | B | | | | D |
| 154a | A | A | B | B | B | B |
| 154b | B | A | | | | C |
| 155a | B | A | C | B | C | B |
| 155b | C | A | | | | C |

The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

With respect to aspects of the invention described as a genus, all individual species are individually considered separate aspects of the invention. If aspects of the invention are described as “comprising” a feature, embodiments also are contemplated “consisting of” or “consisting essentially of” the feature.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference. To the extent that any meaning or definition of a term in this document conflicts with any meaning or definition of the same term in a document incorporated by reference, the meaning or definition assigned to that term in this document shall govern.

1. A compound of Formula I, or a pharmaceutically acceptable salt thereof: 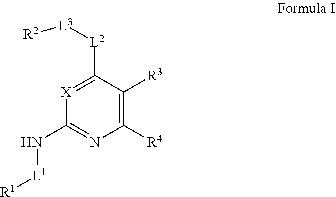
wherein:
L¹ is an optionally substituted phenylene, optionally substituted 5- or 6-membered heteroarylene, optionally substituted 4-8-membered heterocyclylene, or optionally substituted C₃₋₈ carbocyclylene;
R¹ is SO₂R¹⁰, SO₂NR¹¹R¹², S(O)(NH)R¹⁰, or C(O)NR¹¹R¹²;
X is N or CR¹³;
L² is a bond, —N(R¹⁴)—, or —O—;
L³ is a bond, an optionally substituted C₁₋₄ alkylene or an optionally substituted C₁₋₄ heteroalkylene;
R² is hydrogen, an optionally substituted C₃₋₈ alkyl, optionally substituted C₃₋₈ carbocyclyl, optionally substituted 4-10 membered heterocyclyl, optionally substituted phenyl, or optionally substituted 5-10 membered heteroaryl;
R³ is hydrogen, halogen, CN, C(O)NR¹¹R¹², optionally substituted C₁₋₆ alkyl, optionally substituted C₂₋₄ alkenyl, optionally substituted C₂₋₄ alkynyl, optionally substituted C₁₋₄ heteroalkyl, OR^{A}, COR^{B}, COOR^{A}, NR¹¹R¹², optionally substituted C₃₋₈ carbocyclyl, optionally substituted 4-10 membered heterocyclyl, or optionally substituted 5-10 membered heteroaryl;
R⁴ is hydrogen, halogen, optionally substituted C₁₋₆ alkyl, or NR¹¹R¹²; or L² and R³, together with the intervening atoms, form an optionally substituted 4-8 membered ring structure; or R³ and R⁴, together with the intervening atoms, form an optionally substituted 4-8 membered ring structure; wherein:
R¹⁰ is an optionally substituted C₁₋₆ alkyl, optionally substituted C₃₋₈ carbocyclyl, optionally substituted phenyl, optionally substituted 5- or 6-membered heteroaryl, or optionally substituted 4-10 membered heterocyclyl; each of R¹¹ and R¹², at each occurrence, is independently hydrogen, an optionally substituted C₁₋₆ alkyl, optionally substituted C₃₋₈ carbocyclyl, optionally substituted phenyl, optionally substituted 5- or 6-membered heteroaryl, optionally substituted 4-10 membered heterocyclyl; or a nitrogen protecting group; or R¹¹ and R¹² can be joined to form an optionally substituted 4-10 membered heterocyclyl or 5- or 6-membered heteroaryl;
R^{A} is hydrogen, an optionally substituted C₁₋₆ alkyl, optionally substituted C₃₋₈ carbocyclyl, optionally substituted phenyl, optionally substituted 5- or 6-membered heteroaryl, optionally substituted 4-10 membered heterocyclyl; or an oxygen protecting group;
R^{B} is hydrogen, an optionally substituted C₁₋₆ alkyl, optionally substituted C₃₋₈ carbocyclyl, optionally substituted phenyl, optionally substituted 4-10 membered heterocyclyl, or optionally substituted 5- or 6-membered heteroaryl;
R¹³ is hydrogen, F, CN, —OH, an optionally substituted C₁₋₄ alkyl, optionally substituted C₁₋₄ heteroalkyl, optionally substituted C₃₋₈ carbocyclyl, or optionally substituted 4-10 membered heterocyclyl; and
R¹⁴ is hydrogen, an optionally substituted C₁₋₆ alkyl, optionally substituted C₃₋₈ carbocyclyl, optionally substituted phenyl, optionally substituted 5- or 6-membered heteroaryl, optionally substituted 4-10 membered heterocyclyl; or a nitrogen protecting group.

2. The compound of claim 1, or a pharmaceutically acceptable salt thereof, wherein L¹ is selected from: 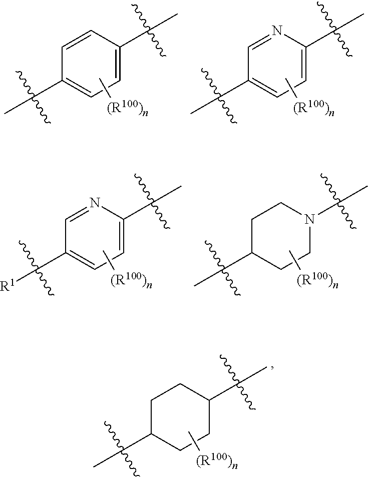
wherein:
n is 0, 1, 2, 3, or 4, as valency permits; and
R¹⁰⁰ at each occurrence is independently selected from halogen CN, OH, optionally substituted C₁₋₄ alkyl, optionally substituted C₁₋₄ alkoxy, and optionally substituted C₁₋₄ heteroalkyl.

3-5. (canceled)

6. The compound of claim 1, or a pharmaceutically acceptable salt thereof, wherein L¹ is
selected from: 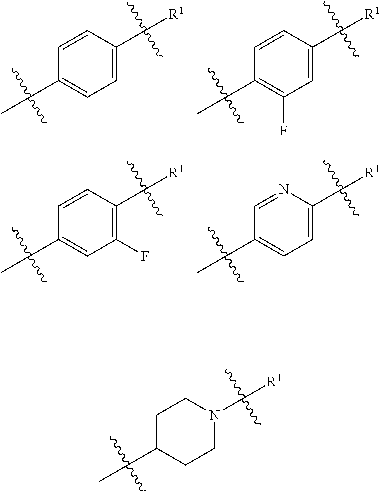
or L¹ is selected from

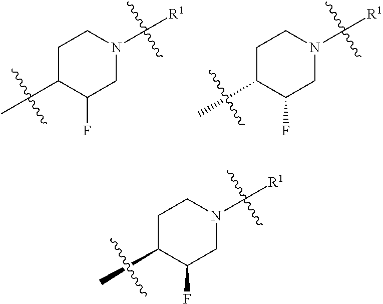

7. The compound of claim 1, or a pharmaceutically acceptable salt thereof, wherein R¹ is SO₂R¹⁰, wherein R¹⁰ is an optionally substituted C₁₋₄ alkyl, optionally substituted C₃₋₆ cycloalkyl, or optionally substituted 4-8 membered heterocyclyl having one or two ring heteroatoms independently selected from N, O, and S, or R¹⁰ is an optionally substituted 5 or 6 membered heteroaryl having 1-3 ring heteroatoms independently selected from N, O, and S.

8. (canceled)

9. The compound of claim 1, or a pharmaceutically acceptable salt thereof,
wherein R¹ is SO₂Me or selected from: 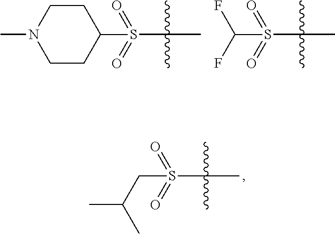
or R¹ is selected from 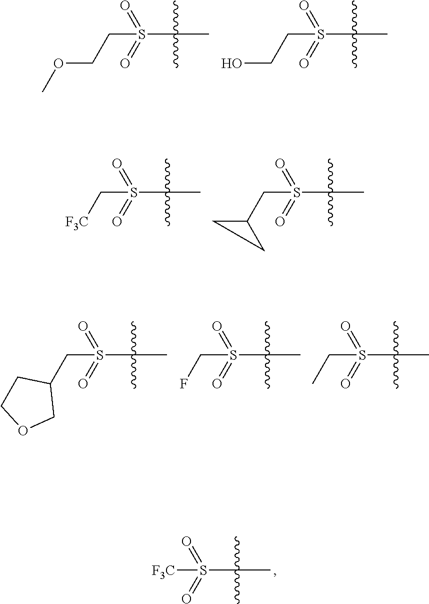
or R¹ is selected from

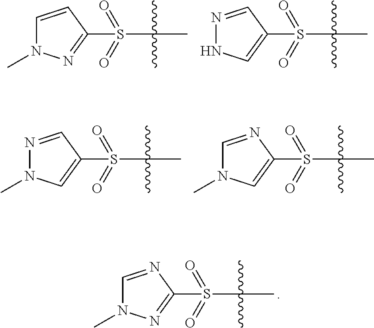

10. The compound of claim 1, or a pharmaceutically acceptable salt thereof, wherein R¹ is S(O)(NH)R¹⁰, wherein R¹⁰ is an optionally substituted C₁₋₄ alkyl, optionally substituted C₃₋₆ cycloalkyl, or optionally substituted 4-8 membered heterocyclyl having one or two ring heteroatoms independently selected from N, O, and S.

11. (canceled)

12. The compound of claim 1, or a pharmaceutically acceptable salt thereof, wherein R¹ is S(O)(NH)Me.

13. The compound of claim 1, or a pharmaceutically acceptable salt thereof, wherein R¹ is SO₂NR¹¹R¹², wherein R¹¹ and R¹² are independently hydrogen, an optionally substituted C₁0.4 alkyl, optionally substituted C₃₋₆ cycloalkyl, or optionally substituted 4-8 membered heterocyclyl having one or two ring heteroatoms independently selected from N, O, and S.

14-16. (canceled)

17. The compound of claim 1, or a pharmaceutically acceptable salt thereof, wherein R¹ is SO₂NH₂ or R¹ is selected from: 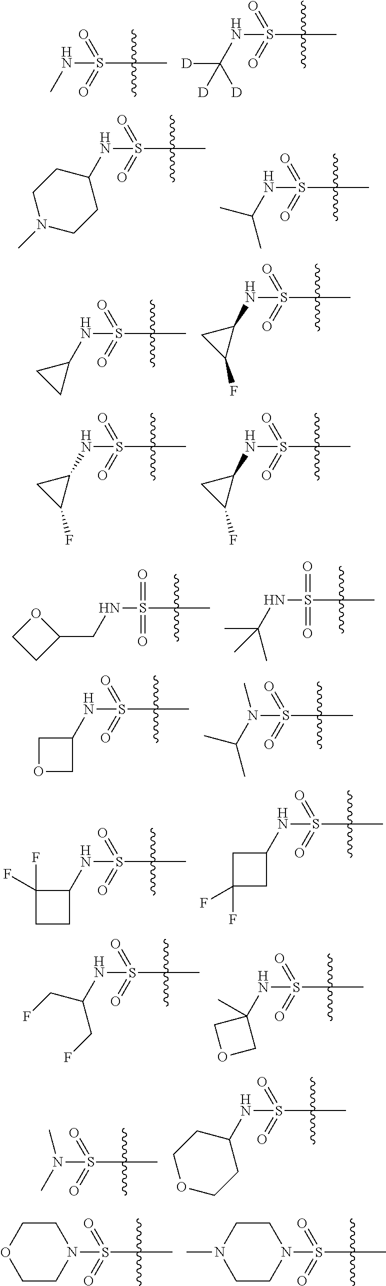
or R¹ is selected from

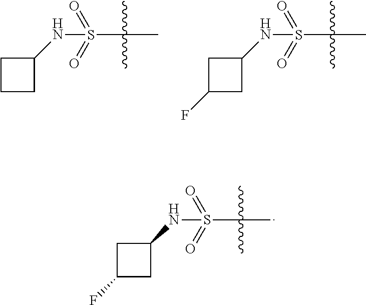

18. The compound of claim 1, or a pharmaceutically acceptable salt thereof, wherein R¹ is C(O)NR¹¹R¹², wherein R¹¹ and R¹² are independently hydrogen, an optionally substituted C₁₋₄ alkyl, optionally substituted C₃₋₆ cycloalkyl, or optionally substituted 4-8 membered heterocyclyl having one or two ring heteroatoms independently selected from N, O, and S.

19-21. (canceled)

22. The compound of claim 1, or a pharmaceutically acceptable salt thereof,
wherein R¹ is C(O)NHMe or

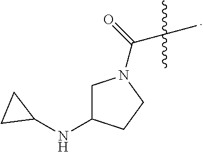 [SMILES: *C(=O)N1CCC(NC2CC2)C1]

23. The compound of claim 1, or a pharmaceutically acceptable salt thereof, wherein L¹-R¹ in Formula I is selected from: 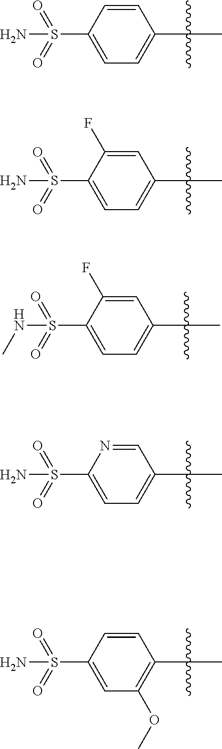 [SMILES: *c1ccc(S(N)(=O)=O)cc1 ; *c1ccc(S(N)(=O)=O)c(F)c1 ; *c1ccc(S(=O)(=O)NC)c(F)c1 ; *c1ccc(S(N)(=O)=O)nc1 ; *c1ccc(S(N)(=O)=O)cc1OC]
or L¹-R¹ is 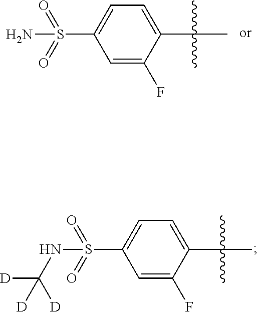
or L¹-R¹ in Formula I is selected from: 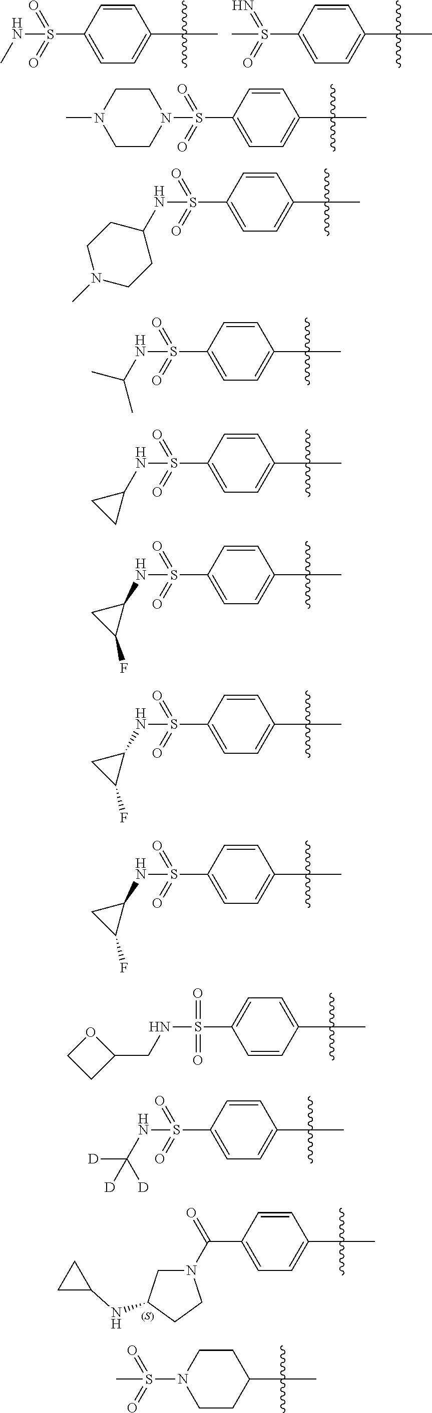 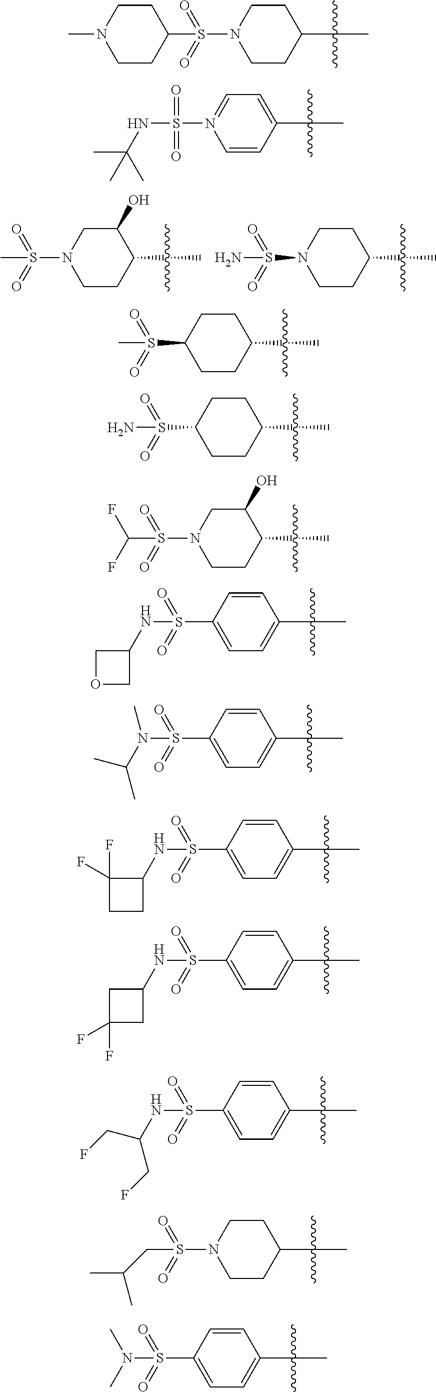 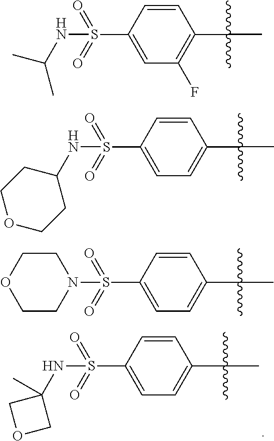
or L¹-R¹ in Formula I is selected from:

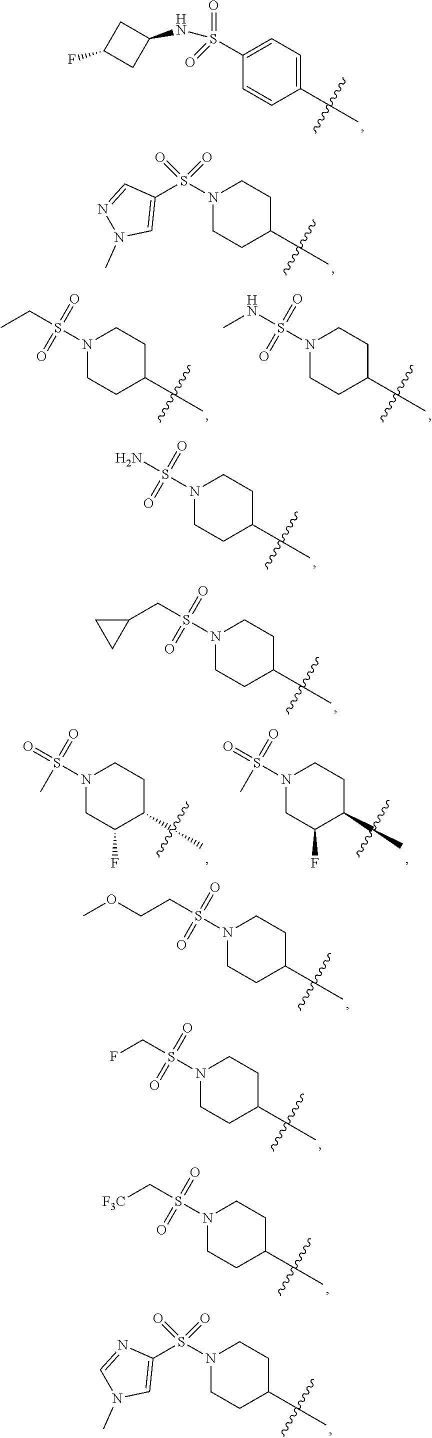

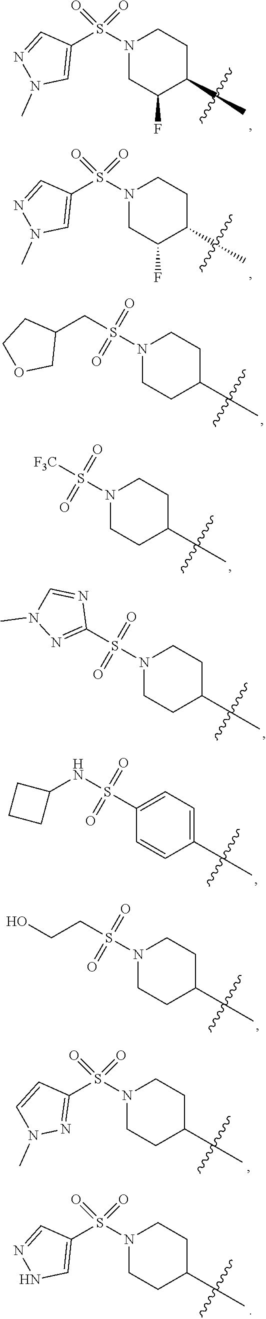

24. (canceled)

25. The compound of claim 1, or a pharmaceutically acceptable salt thereof, wherein X is N or CH.

26. (canceled)

27. The compound of claim 1, or a pharmaceutically acceptable salt thereof, wherein L² is —O— and L³ is a bond or a C₁₋₄ alkylene optionally substituted with one or more substituents independently selected from F, OH, and protected OH.

28. The compound of claim 27, or a pharmaceutically acceptable salt thereof, characterized as having Formula I-1 or I-2:

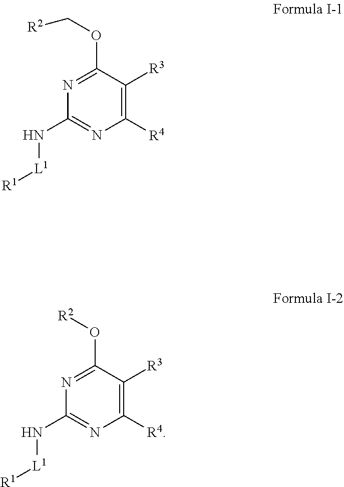

29. The compound of claim 1, or a pharmaceutically acceptable salt thereof, wherein L² is —N(R¹⁴)- and L³ is a bond or a C₁₋₄ alkylene optionally substituted with one or more substituents independently selected from F, OH, and protected OH.

30. (canceled)

31. The compound of claim 29, or a pharmaceutically acceptable salt thereof, characterized as having Formula I-3 or I-4:

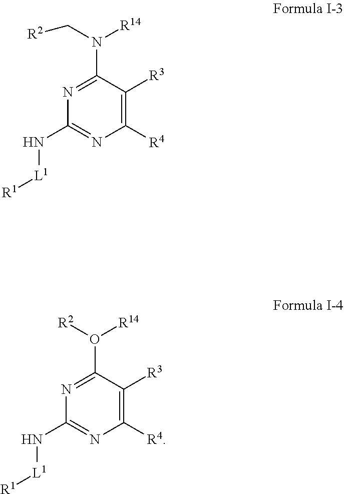

32. The compound of claim 1, or a pharmaceutically acceptable salt thereof, wherein R² is a C₃₋₈ alkyl substituted with one or more substituents independently selected from oxo, F, G¹, CN, OH, O-G¹, NH₂, NH(G¹), and N(G¹)(G¹), wherein G¹ at each occurrence is independently a C₁₋₄ alkyl optionally substituted with 1-3 substituents independently selected from F, CN, OH, and C₁₋₄ heteroalkyl; or a C₃₋₆ cycloalkyl optionally substituted with 1-3 substituents independently selected from F, CN, OH, and C₁₋₄ heteroalkyl, wherein two optional substituents of the C₃₋₈ alkyl, together with the intervening atom(s), can optionally be joined to form a ring structure: or a 4-6 or 7 membered monocyclic heterocyclyl having 1-2 ring heteroatoms independently selected from N, O, and S, which is optionally substituted with one or more substituents independently selected from oxo, F, methyl, ethyl, hydroxyethyl, fluorine substituted methyl, and fluorine substituted ethyl.

33. The compound of claim 32, or a pharmaceutically acceptable salt thereof, wherein R² is selected from: 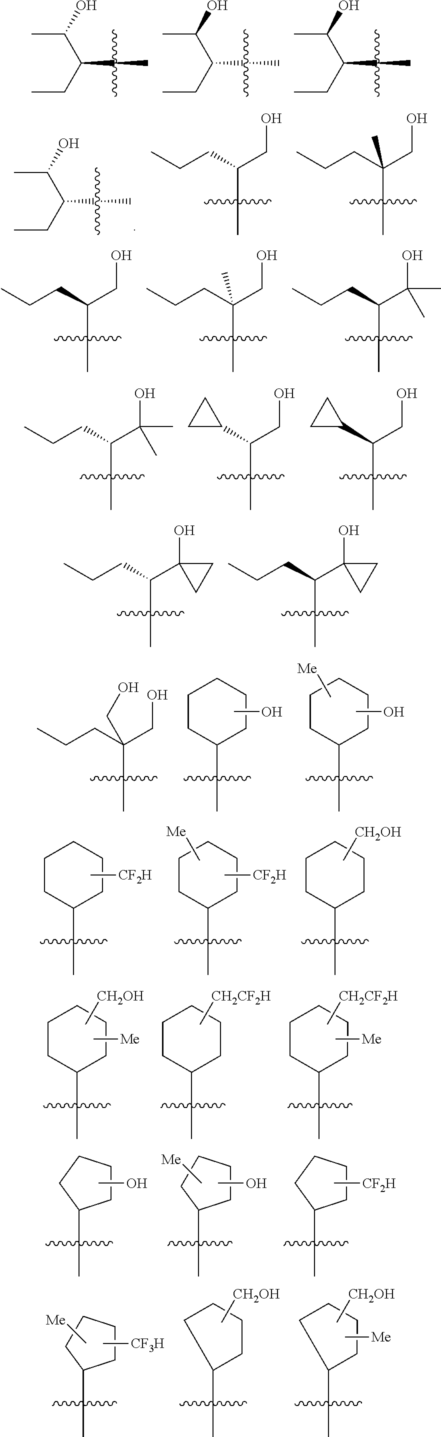 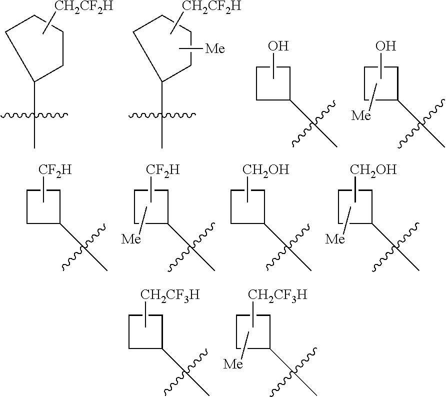
or R² is selected from: 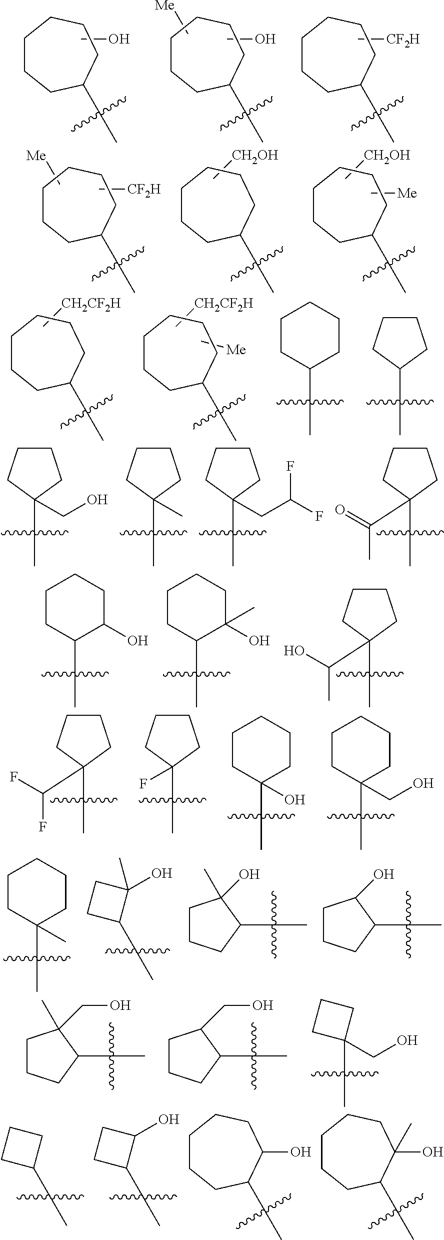
or R² is selected from: 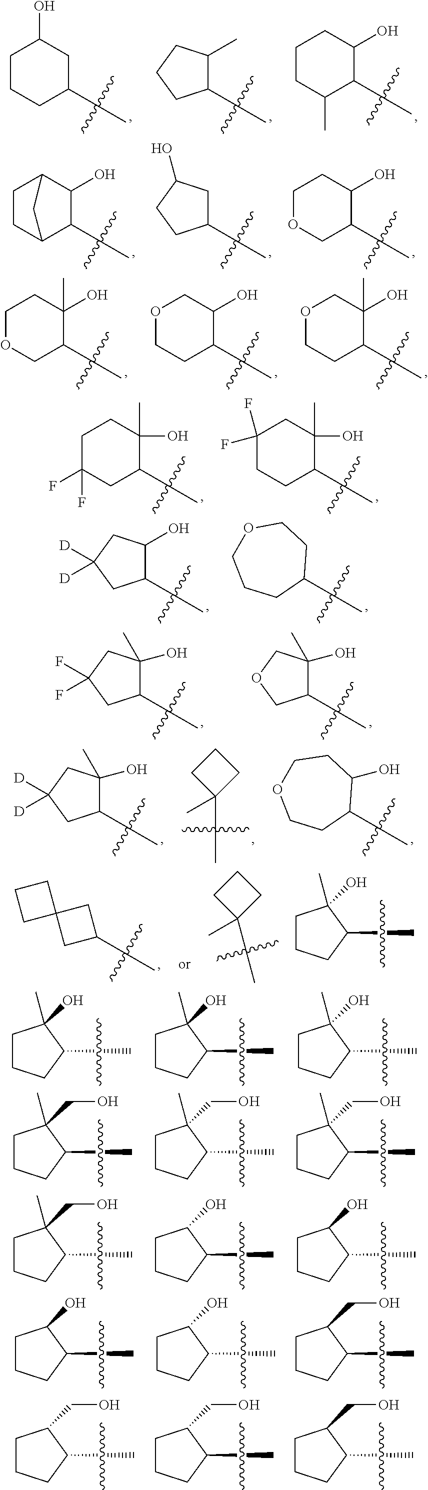 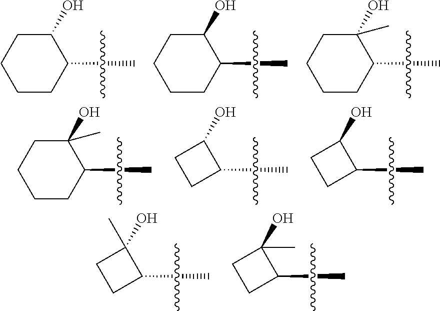
or R² is selected from: 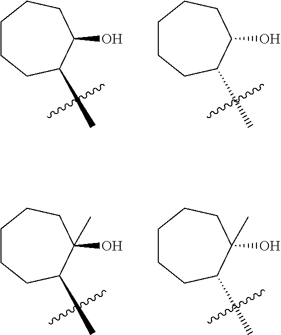
or R² is selected from:

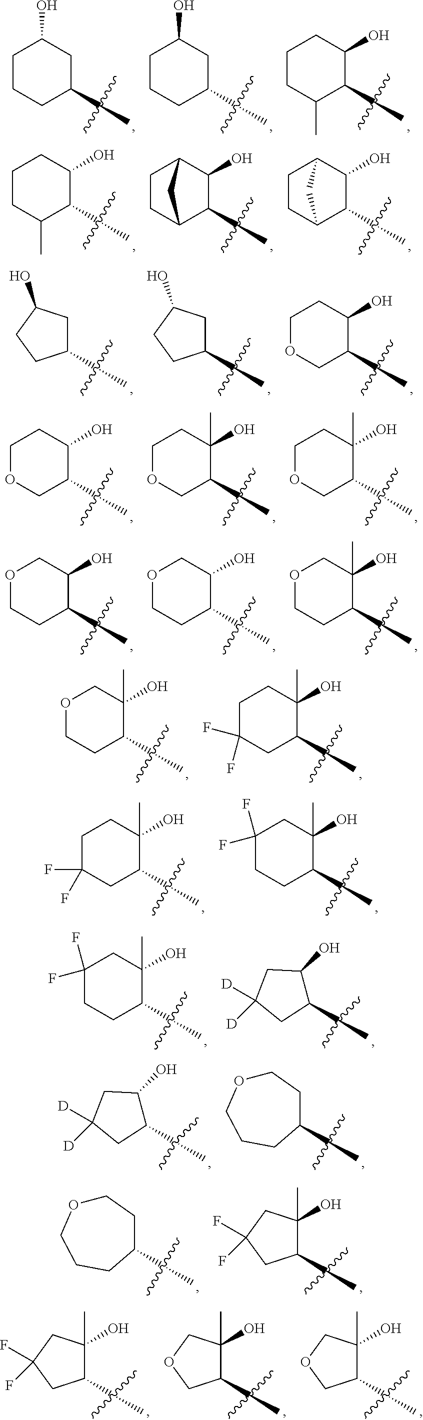

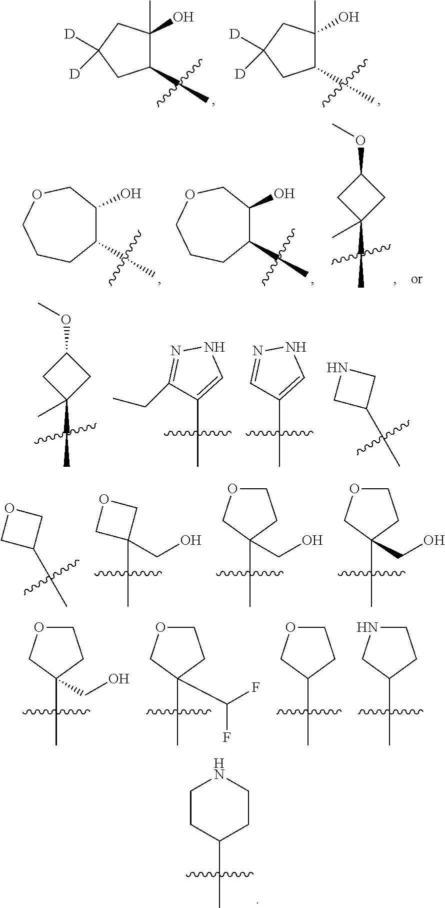

34-43. (canceled)

44. The compound of claim 1, or a pharmaceutically acceptable salt thereof, wherein L² and L³ are both a bond.

45. The compound of claim 44, or a pharmaceutically acceptable salt thereof, characterized as having Formula I-5:

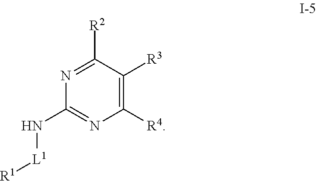

46-47. (canceled)

48. The compound of claim 44, or a pharmaceutically acceptable salt thereof, wherein R² is selected from: 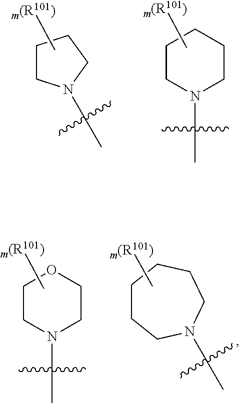
wherein:
m is 0, 1, 2, 3, or 4;
R¹⁰¹ at each occurrence is independently oxo, F, CN, G¹, G², OH, O-G¹, and O-G², wherein G¹ at each occurrence is independently a C₁₋₄ alkyl optionally substituted with 1-3 substituents independently selected from F, CN, OH, and C₁₋₄ heteroalkyl, or a C₃₋₆ cycloalkyl optionally substituted with 1-3 substituents independently selected from F, CN, OH, and C₁₋₄ heteroalkyl; wherein G² at each occurrence is independently 4-6 membered heterocyclyl having 1-2 ring heteroatoms independently selected from N, O, and S, phenyl or 5- or 6-membered heteroaryl having 1-4 ring heteroatoms independently selected from N, O, and S, each of which is optionally substituted with 1-3 substituents independently selected from F, CN, G¹, OH, and O-G¹; wherein two R¹⁰¹, together with the intervening atom(s), can optionally be joined to form a fused, bridged, or spiro ring structure:
or wherein R² is: 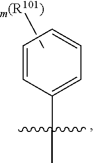
wherein:
m is 0, 1, 2, or 3:
R¹⁰¹ at each occurrence is independently F, CN, G¹, G², OH, O-G¹, O-G², NH₂, NH(G¹), NH(G²), N(G¹)(G¹), and N(G¹)(G²), wherein G¹ at each occurrence is independently a C₁₋₄ alkyl optionally substituted with 1-3 substituents independently selected from F, OH, and C₁₋₄ heteroalkyl or a C₃₋₆ cycloalkyl optionally substituted with 1-3 substituents independently selected from F, OH, and C₁₋₄ heteroalkyl; wherein G² at each occurrence is independently 4-6 membered heterocyclyl having 1-2 ring heteroatoms independently selected from N, O, and S, phenyl or 5- or 6-membered heteroaryl having 1-4 ring heteroatoms independently selected from N, O, and S, each of which is optionally substituted with 1-3 substituents independently selected from F, CN, G¹, OH, and O-G¹; wherein two R¹⁰¹, together with the intervening atom(s), can optionally be joined to form a fused ring structure.

49-50. (canceled)

51. The compound of claim 44, or a pharmaceutically acceptable salt thereof, wherein R² is selected from:

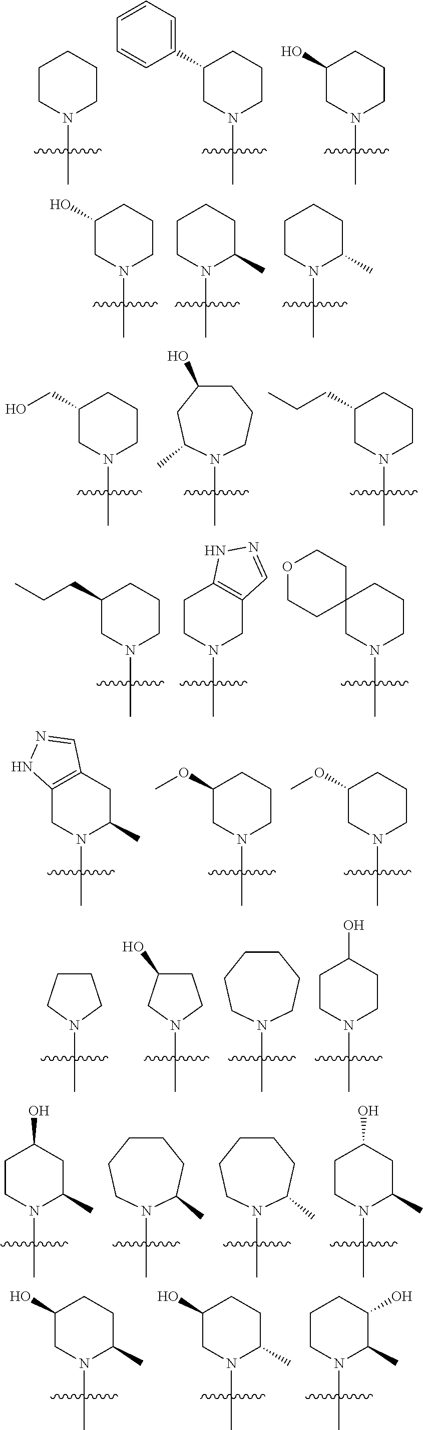

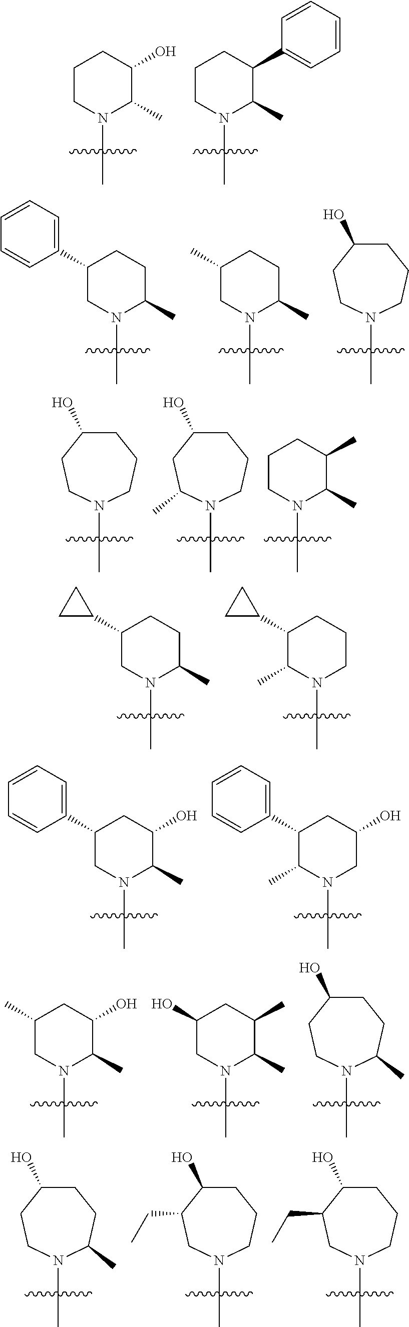

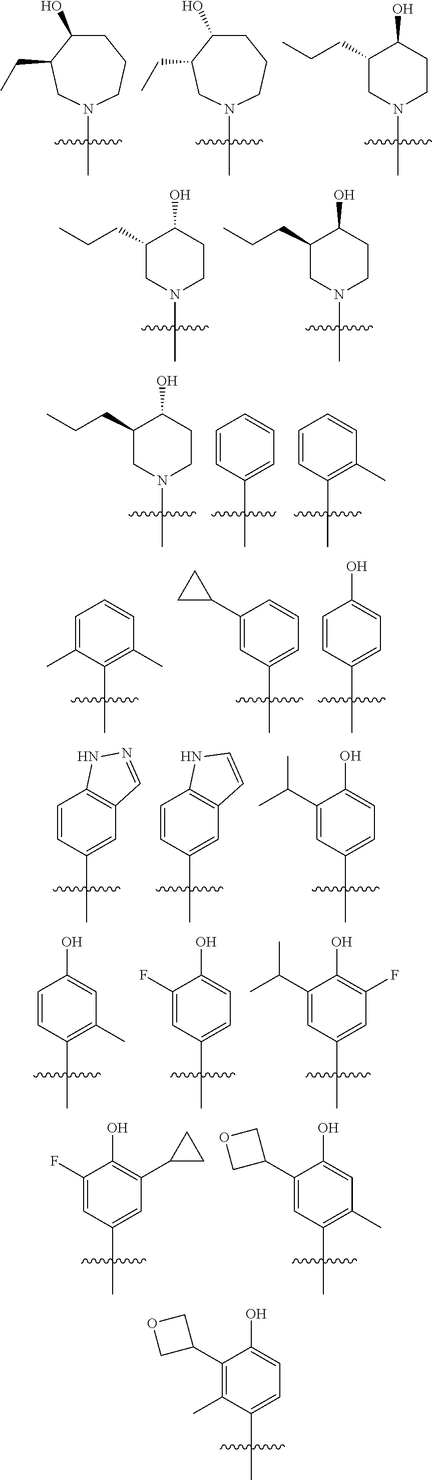

52-61. (canceled)

62. The compound of claim 1, or a pharmaceutically acceptable salt thereof wherein R³ is selected from:

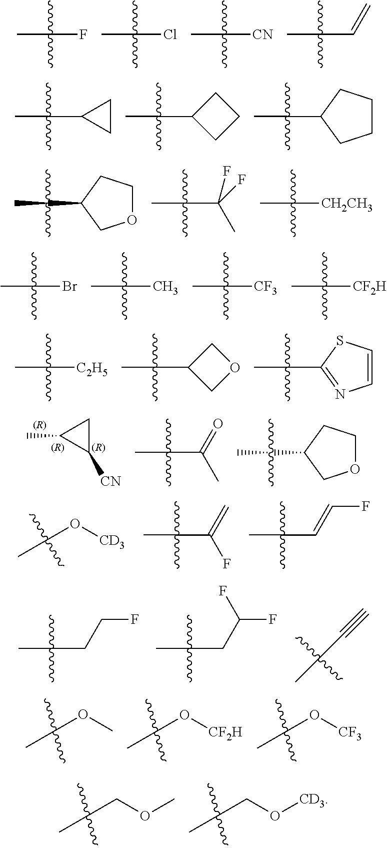

63. The compound of claim 1, or a pharmaceutically acceptable salt thereof wherein R⁴ is hydrogen or NH₂.

64-65. (canceled)

66. The compound of claim 1, or a pharmaceutically acceptable salt thereof wherein R³ and R⁴ are joined to form

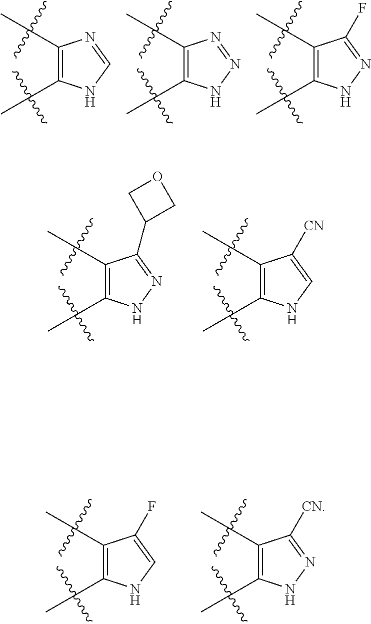

67. A compound of Formula II, or a pharmaceutically acceptable salt thereof: 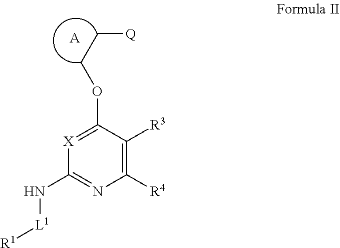
wherein:
L¹ is an optionally substituted phenylene, optionally substituted 5- or 6-membered heteroarylene, optionally substituted 4-8-membered heterocyclylene, or optionally substituted C₃₋₈ carbocyclylene;
R¹ is SO₂R¹⁰, SO₂NR¹¹R¹², S(O)(NH)R¹⁰, or C(O)NR¹¹R¹²;
X is N or CR¹³;
Ring A is an optionally substituted carbocyclic ring or optionally substituted heterocyclic ring having one or more ring heteroatoms independently selected from O, N, and S;
Q is hydrogen, OR^{A}, optionally substituted C¹⁻⁴ alkyl, halogen, CN, or COR^{B}; R³ is hydrogen, halogen, CN, C(O)NR¹¹R¹², optionally substituted C₁₋₆ alkyl, optionally substituted C₂₋₄ alkenyl, optionally substituted C₂₋₄ alkynyl, optionally substituted C₁₋₄ heteroalkyl, OR^{A}, COR^{B}, COOR^{A}, NR¹¹R¹², optionally substituted C₃₋₈ carbocyclyl, optionally substituted 4-10 membered heterocyclyl, or optionally substituted 5-10 membered heteroaryl;
R⁴ is hydrogen, halogen, optionally substituted C₁₋₆ alkyl, or NR¹¹R¹²; or R³ and R⁴, together with the intervening atoms, form an optionally substituted 4-8 membered ring structure;
wherein:
R¹⁰ is an optionally substituted C₁₋₆ alkyl, optionally substituted C₃₋₈ carbocyclyl, optionally substituted phenyl, optionally substituted 5- or 6-membered heteroaryl, or optionally substituted 4-10 membered heterocyclyl; each of R¹¹ and R¹², at each occurrence, is independently hydrogen, an optionally substituted C₁₋₆ alkyl, optionally substituted C₃₋₈ carbocyclyl, optionally substituted phenyl, optionally substituted 5- or 6-membered heteroaryl), optionally substituted 4-10 membered heterocyclyl; or a nitrogen protecting group; or R¹¹ and R¹² can be joined to form an optionally substituted 4-10 membered heterocyclyl or 5- or 6-membered heteroaryl;
R^{A} at each occurrence is independently hydrogen, an optionally substituted C₁₋₆ alkyl, optionally substituted C₃₋₈ carbocyclyl, optionally substituted phenyl, optionally substituted 5- or 6-membered heteroaryl, optionally substituted 4-10 membered heterocyclyl; or an oxygen protecting group;
R^{B} at each occurrence is independently hydrogen, an optionally substituted C₁₋₆ alkyl, optionally substituted C₃₋₈ carbocyclyl, optionally substituted phenyl, optionally substituted 4-10 membered heterocyclyl, or optionally substituted 5- or 6-membered heteroaryl; and
R¹³ is hydrogen, F, CN, —OH, an optionally substituted C₁₋₄ alkyl, optionally substituted C₁₋₄ heteroalkyl, optionally substituted C₃₋₈ carbocyclyl, or optionally substituted 4-10 membered heterocyclyl.

68-75. (canceled)

76. The compound of claim 67, or a pharmaceutically acceptable salt thereof, wherein 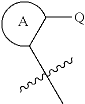
in Formula II is selected from: 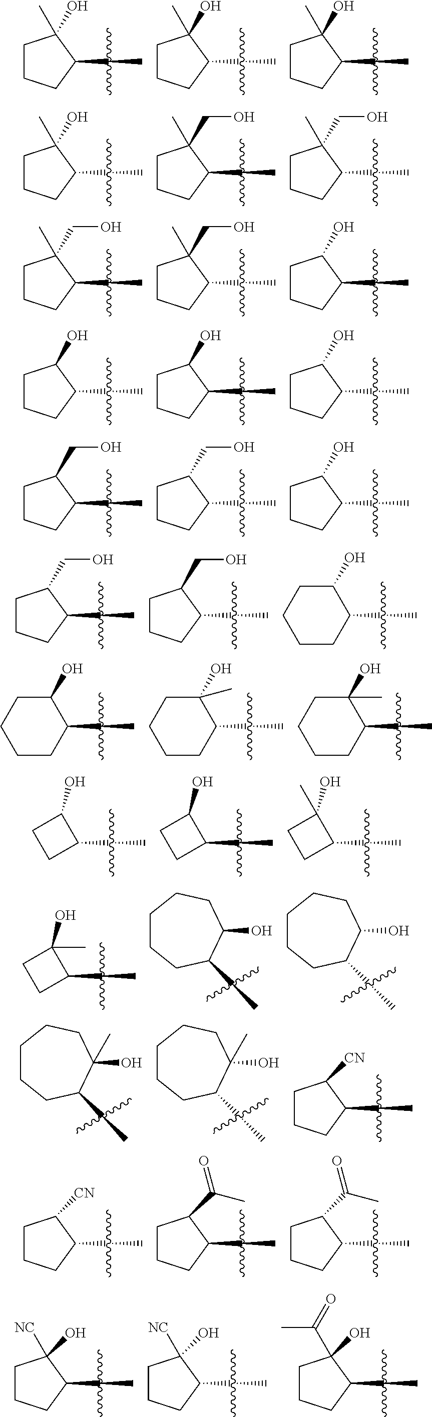 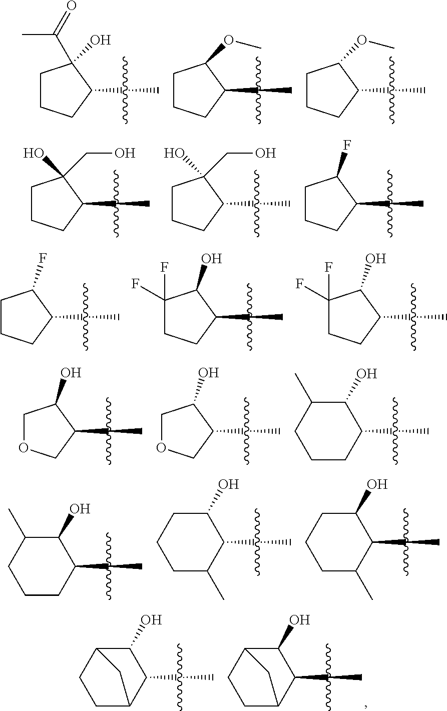
or 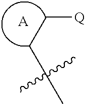
in Formula II is selected from:

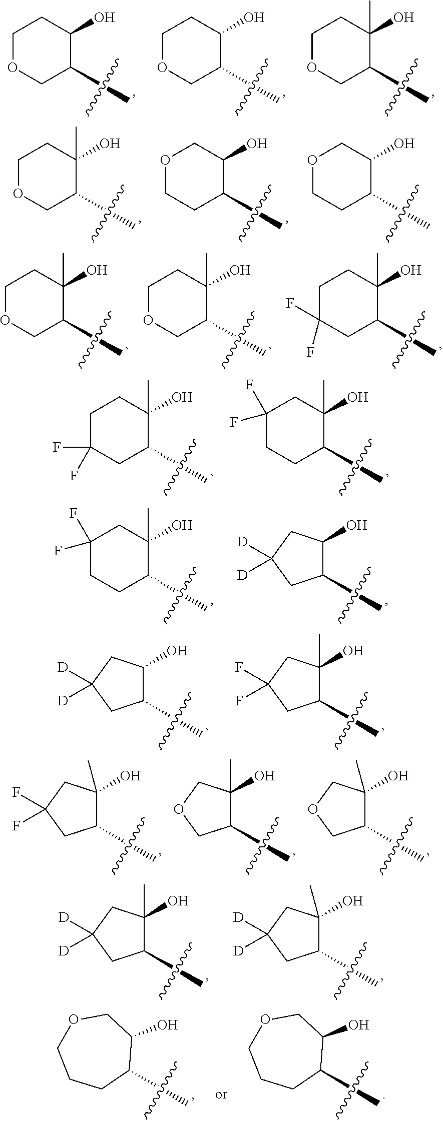

77. The compound claim 67, or a pharmaceutically acceptable salt thereof, characterized as having the following Formula II-1 or II-2: 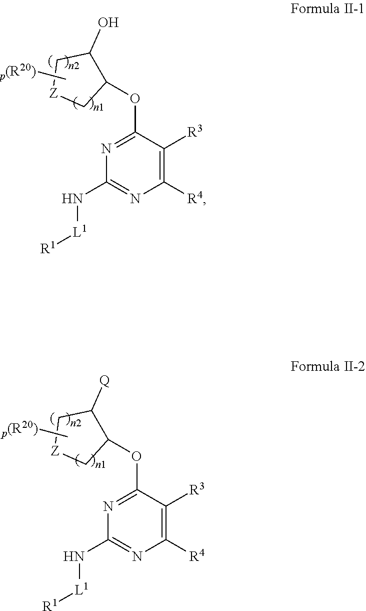
wherein:
n1 and n2 are independently 0, 1, 2, or 3,
Z is CR²¹R²², O, or NR²³
p is 0, 1, 2, 3, or 4, as valency permits,
R²⁰ at each occurrence is independently oxo, halogen, CN, G¹, C(O)H, C(O)G¹, OH, O-G¹, NH₂, NH(G¹), and N(G¹)(G¹), wherein G¹ at each occurrence is independently a C₁₋₄ alkyl optionally substituted with 1-3 substituents independently selected from F, CN, OH, and C₁₋₄ heteroalkyl, or a C₃₋₆ cycloalkyl optionally substituted with 1-3 substituents independently selected from F, CN, OH, and C₁₋₄ heteroalkyl, or two geminal R²⁰ form an oxo group, or two R²⁰ together with the intervening atoms form an optionally substituted ring structure,
R²¹ and R²² are each independently hydrogen or R²⁰, or R²¹ and R²² together form an oxo group or an optionally substituted ring structure, or one of R²¹ and R²² with one R²⁰ group together with the intervening atoms form an optionally substituted ring structure, R²³ is hydrogen or R²⁰,
or R²³ and one R²⁰ group together with the intervening atoms form an optionally substituted ring structure,
wherein Q, L¹, R¹, and R³ are as defined in claim 67.

78-96. (canceled)

97. A compound selected from 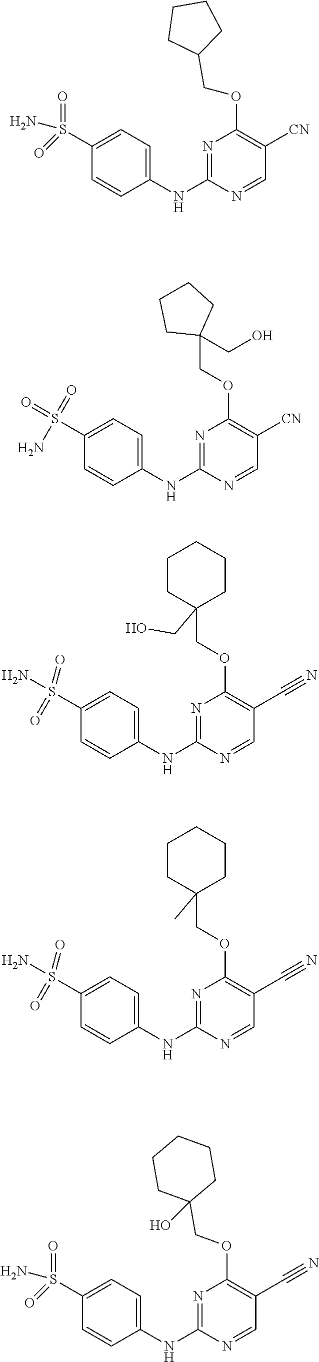 [SMILES: N#Cc1cnc(Nc2ccc(S(N)(=O)=O)cc2)nc1OCC1CCCC1 ; N#Cc1cnc(Nc2ccc(S(N)(=O)=O)cc2)nc1OCC1(CO)CCCC1 ; N#Cc1cnc(Nc2ccc(S(N)(=O)=O)cc2)nc1OCC1(CO)CCCCC1 ; CC1(COc2nc(Nc3ccc(S(N)(=O)=O)cc3)ncc2C#N)CCCCC1 ; N#Cc1cnc(Nc2ccc(S(N)(=O)=O)cc2)nc1OCC1(O)CCCCC1] 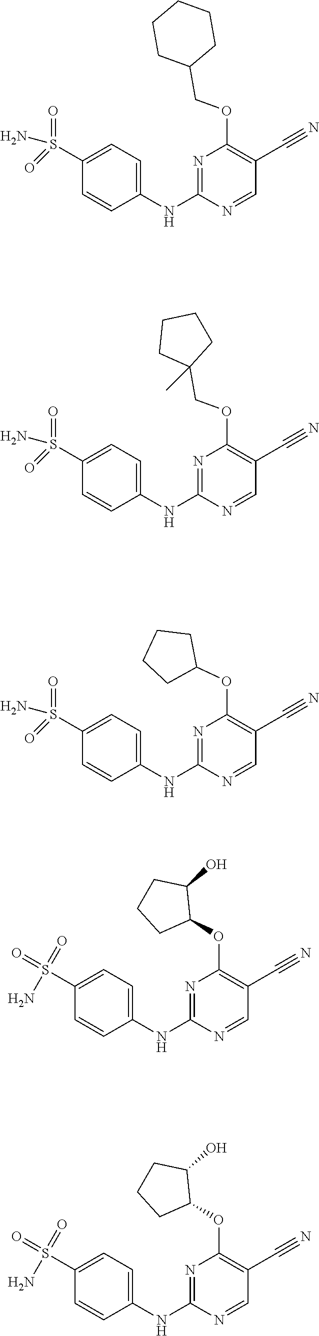 [SMILES: N#Cc1cnc(Nc2ccc(S(N)(=O)=O)cc2)nc1OCC1CCCCC1 ; CC1(COc2nc(Nc3ccc(S(N)(=O)=O)cc3)ncc2C#N)CCCC1 ; N#Cc1cnc(Nc2ccc(S(N)(=O)=O)cc2)nc1OC1CCCC1 ; N#Cc1cnc(Nc2ccc(S(N)(=O)=O)cc2)nc1O[C@H]1CCC[C@H]1O ; N#Cc1cnc(Nc2ccc(S(N)(=O)=O)cc2)nc1O[C@@H]1CCC[C@@H]1O] 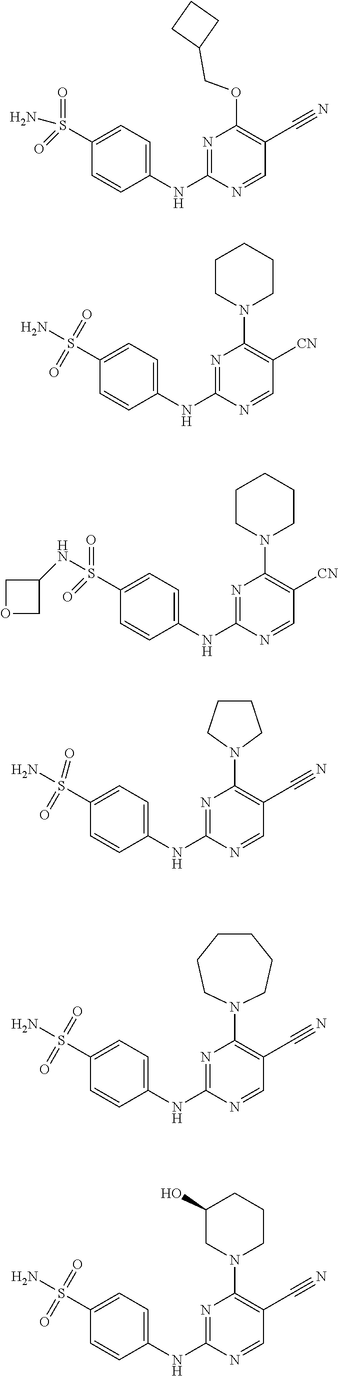 [SMILES: N#Cc1cnc(Nc2ccc(S(N)(=O)=O)cc2)nc1OCC1CCC1 ; N#Cc1cnc(Nc2ccc(S(N)(=O)=O)cc2)nc1N1CCCCC1 ; N#Cc1cnc(Nc2ccc(S(=O)(=O)NC3COC3)cc2)nc1N1CCCCC1 ; N#Cc1cnc(Nc2ccc(S(N)(=O)=O)cc2)nc1N1CCCC1 ; N#Cc1cnc(Nc2ccc(S(N)(=O)=O)cc2)nc1N1CCCCCC1 ; N#Cc1cnc(Nc2ccc(S(N)(=O)=O)cc2)nc1N1CCC[C@H](O)C1] 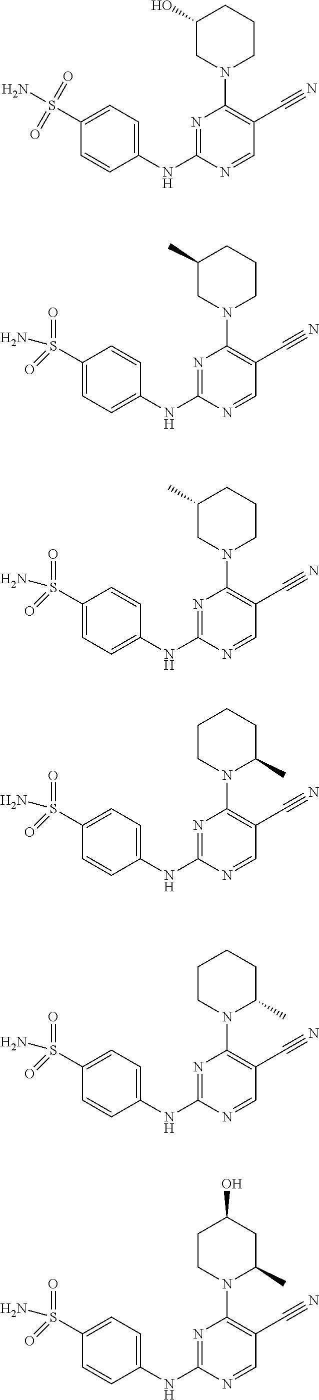 [SMILES: N#Cc1cnc(Nc2ccc(S(N)(=O)=O)cc2)nc1N1CCC[C@@H](O)C1 ; C[C@H]1CCCN(c2nc(Nc3ccc(S(N)(=O)=O)cc3)ncc2C#N)C1 ; C[C@@H]1CCCN(c2nc(Nc3ccc(S(N)(=O)=O)cc3)ncc2C#N)C1 ; C[C@@H]1CCCCN1c1nc(Nc2ccc(S(N)(=O)=O)cc2)ncc1C#N ; C[C@H]1CCCCN1c1nc(Nc2ccc(S(N)(=O)=O)cc2)ncc1C#N ; C[C@@H]1C[C@H](O)CCN1c1nc(Nc2ccc(S(N)(=O)=O)cc2)ncc1C#N] 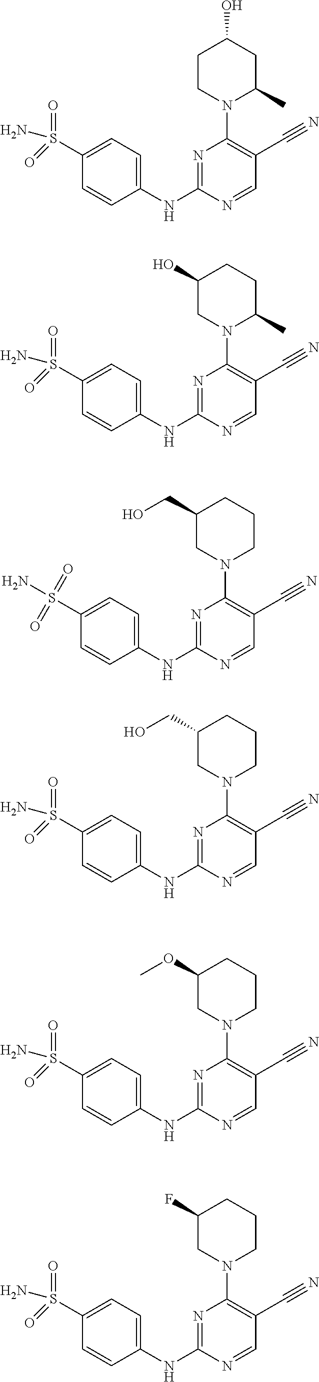 [SMILES: C[C@@H]1C[C@@H](O)CCN1c1nc(Nc2ccc(S(N)(=O)=O)cc2)ncc1C#N ; C[C@@H]1CC[C@H](O)CN1c1nc(Nc2ccc(S(N)(=O)=O)cc2)ncc1C#N ; N#Cc1cnc(Nc2ccc(S(N)(=O)=O)cc2)nc1N1CCC[C@H](CO)C1 ; N#Cc1cnc(Nc2ccc(S(N)(=O)=O)cc2)nc1N1CCC[C@@H](CO)C1 ; CO[C@H]1CCCN(c2nc(Nc3ccc(S(N)(=O)=O)cc3)ncc2C#N)C1 ; N#Cc1cnc(Nc2ccc(S(N)(=O)=O)cc2)nc1N1CCC[C@H](F)C1] 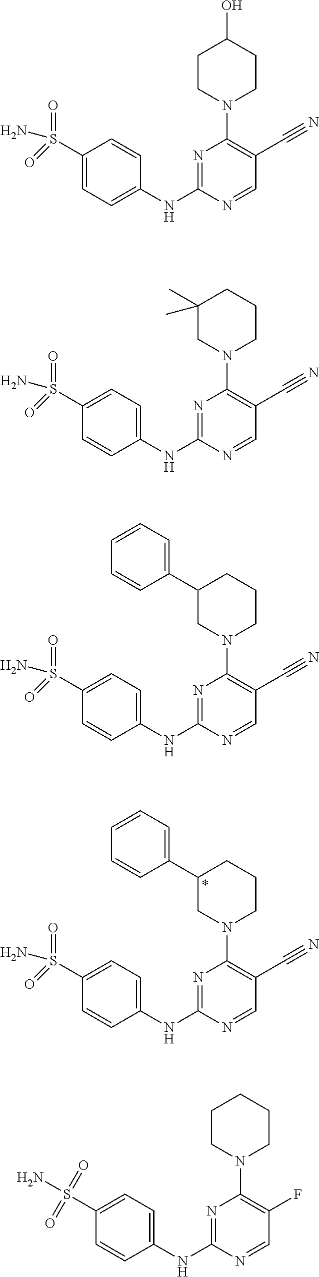 [SMILES: N#Cc1cnc(Nc2ccc(S(N)(=O)=O)cc2)nc1N1CCC(O)CC1 ; CC1(C)CCCN(c2nc(Nc3ccc(S(N)(=O)=O)cc3)ncc2C#N)C1 ; N#Cc1cnc(Nc2ccc(S(N)(=O)=O)cc2)nc1N1CCCC(c2ccccc2)C1 ; N#Cc1cnc(Nc2ccc(S(N)(=O)=O)cc2)nc1N1CCCC(c2ccccc2)C1 ; NS(=O)(=O)c1ccc(Nc2ncc(F)c(N3CCCCC3)n2)cc1] 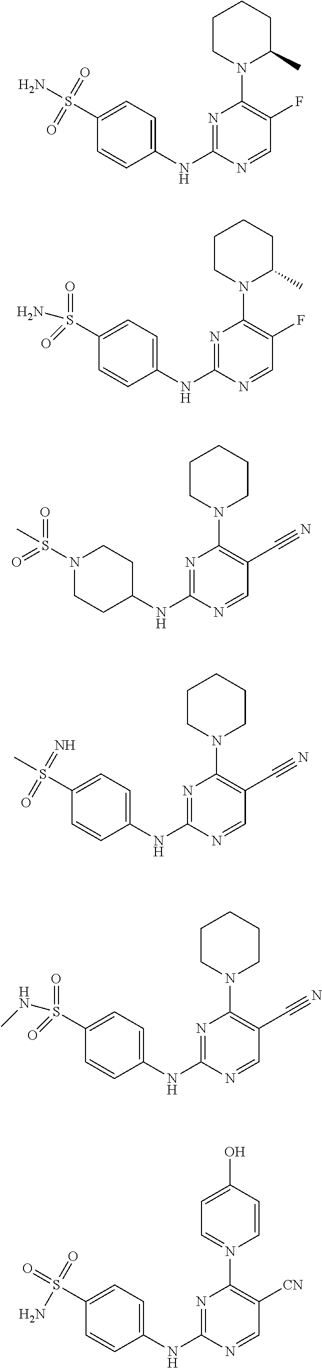 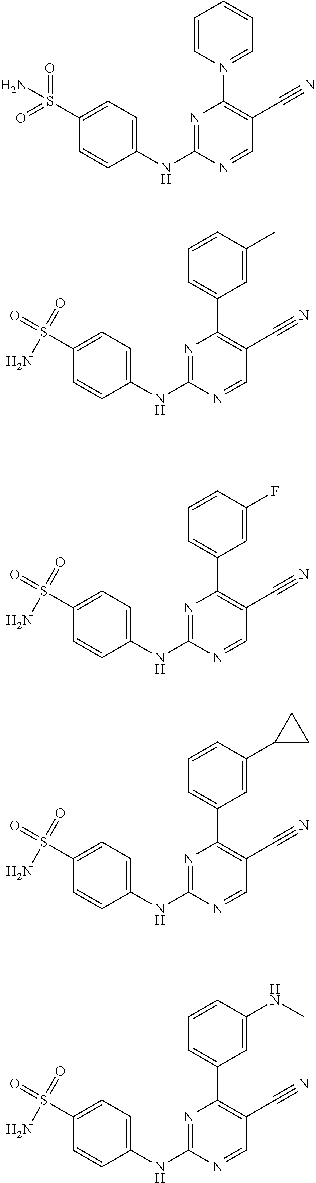 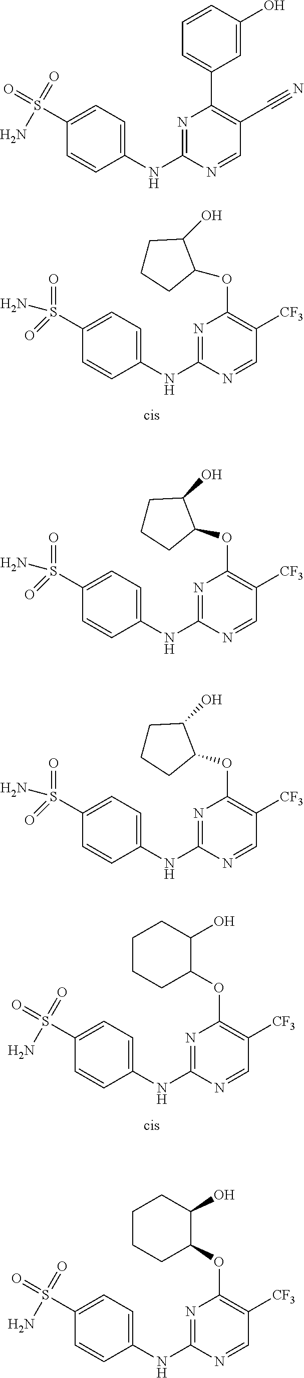 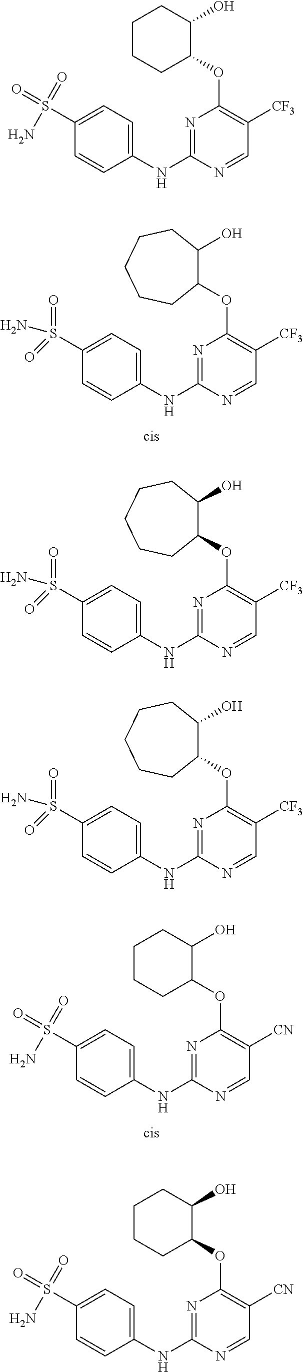 [SMILES: NS(=O)(=O)c1ccc(Nc2ncc(C(F)(F)F)c(O[C@@H]3CCCC[C@@H]3O)n2)cc1 ; NS(=O)(=O)c1ccc(Nc2ncc(C(F)(F)F)c(OC3CCCCCC3O)n2)cc1 ; NS(=O)(=O)c1ccc(Nc2ncc(C(F)(F)F)c(O[C@H]3CCCCC[C@H]3O)n2)cc1 ; NS(=O)(=O)c1ccc(Nc2ncc(C(F)(F)F)c(O[C@@H]3CCCCC[C@@H]3O)n2)cc1 ; N#Cc1cnc(Nc2ccc(S(N)(=O)=O)cc2)nc1OC1CCCCC1O ; N#Cc1cnc(Nc2ccc(S(N)(=O)=O)cc2)nc1O[C@H]1CCCC[C@H]1O] 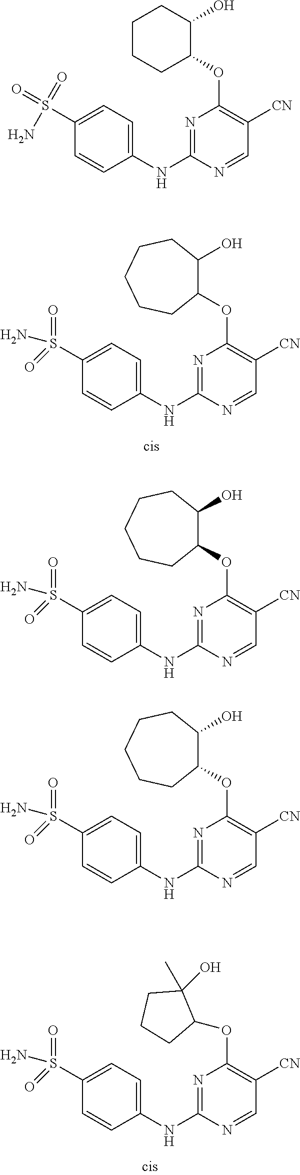 [SMILES: N#Cc1cnc(Nc2ccc(S(N)(=O)=O)cc2)nc1O[C@@H]1CCCC[C@@H]1O ; N#Cc1cnc(Nc2ccc(S(N)(=O)=O)cc2)nc1OC1CCCCCC1O ; N#Cc1cnc(Nc2ccc(S(N)(=O)=O)cc2)nc1O[C@H]1CCCCC[C@H]1O ; N#Cc1cnc(Nc2ccc(S(N)(=O)=O)cc2)nc1O[C@@H]1CCCCC[C@@H]1O ; CC1(O)CCCC1Oc1nc(Nc2ccc(S(N)(=O)=O)cc2)ncc1C#N] 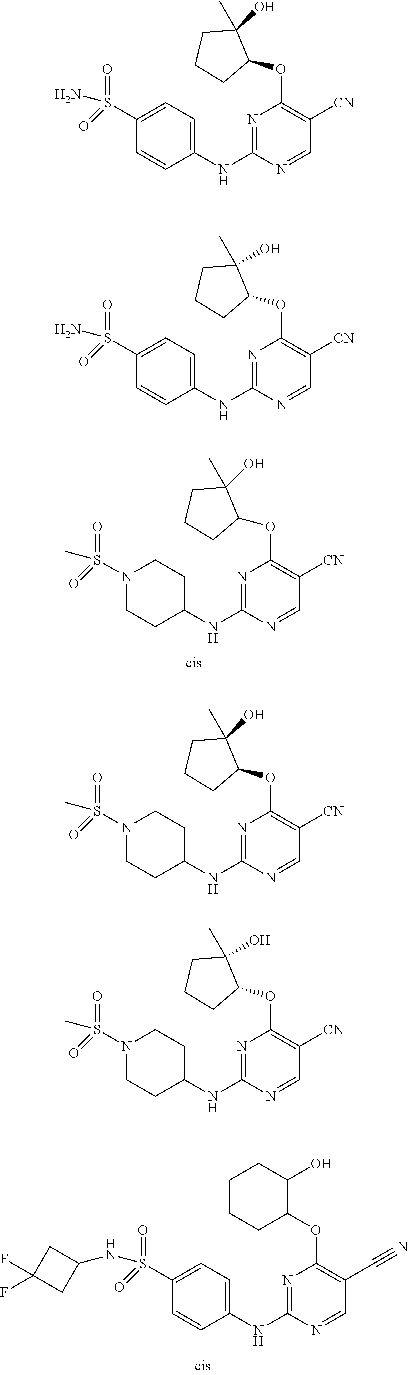 [SMILES: C[C@@]1(O)CCC[C@@H]1Oc1nc(Nc2ccc(S(N)(=O)=O)cc2)ncc1C#N ; C[C@]1(O)CCC[C@H]1Oc1nc(Nc2ccc(S(N)(=O)=O)cc2)ncc1C#N ; CC1(O)CCCC1Oc1nc(NC2CCN(S(C)(=O)=O)CC2)ncc1C#N ; C[C@@]1(O)CCC[C@@H]1Oc1nc(NC2CCN(S(C)(=O)=O)CC2)ncc1C#N ; C[C@]1(O)CCC[C@H]1Oc1nc(NC2CCN(S(C)(=O)=O)CC2)ncc1C#N ; N#Cc1cnc(Nc2ccc(S(=O)(=O)NC3CC(F)(F)C3)cc2)nc1OC1CCCCC1O] 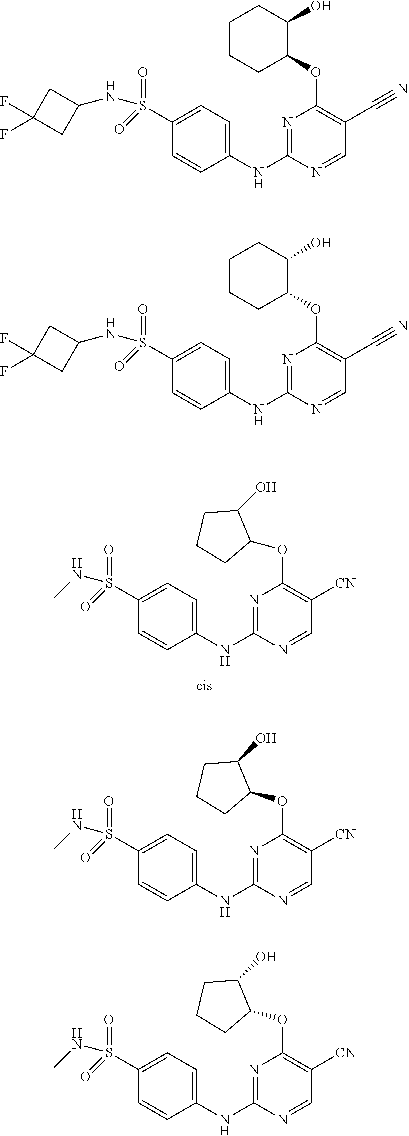 [SMILES: N#Cc1cnc(Nc2ccc(S(=O)(=O)NC3CC(F)(F)C3)cc2)nc1O[C@H]1CCCC[C@H]1O ; N#Cc1cnc(Nc2ccc(S(=O)(=O)NC3CC(F)(F)C3)cc2)nc1O[C@@H]1CCCC[C@@H]1O ; CNS(=O)(=O)c1ccc(Nc2ncc(C#N)c(OC3CCCC3O)n2)cc1 ; CNS(=O)(=O)c1ccc(Nc2ncc(C#N)c(O[C@H]3CCC[C@H]3O)n2)cc1 ; CNS(=O)(=O)c1ccc(Nc2ncc(C#N)c(O[C@@H]3CCC[C@@H]3O)n2)cc1] 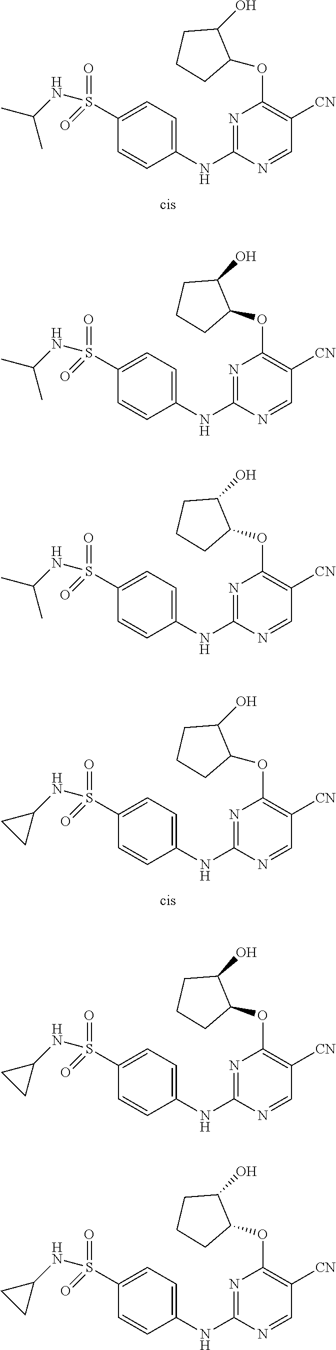 [SMILES: CC(C)NS(=O)(=O)c1ccc(Nc2ncc(C#N)c(OC3CCCC3O)n2)cc1 ; CC(C)NS(=O)(=O)c1ccc(Nc2ncc(C#N)c(O[C@H]3CCC[C@H]3O)n2)cc1 ; CC(C)NS(=O)(=O)c1ccc(Nc2ncc(C#N)c(O[C@@H]3CCC[C@@H]3O)n2)cc1 ; N#Cc1cnc(Nc2ccc(S(=O)(=O)NC3CC3)cc2)nc1OC1CCCC1O ; N#Cc1cnc(Nc2ccc(S(=O)(=O)NC3CC3)cc2)nc1O[C@H]1CCC[C@H]1O ; N#Cc1cnc(Nc2ccc(S(=O)(=O)NC3CC3)cc2)nc1O[C@@H]1CCC[C@@H]1O] 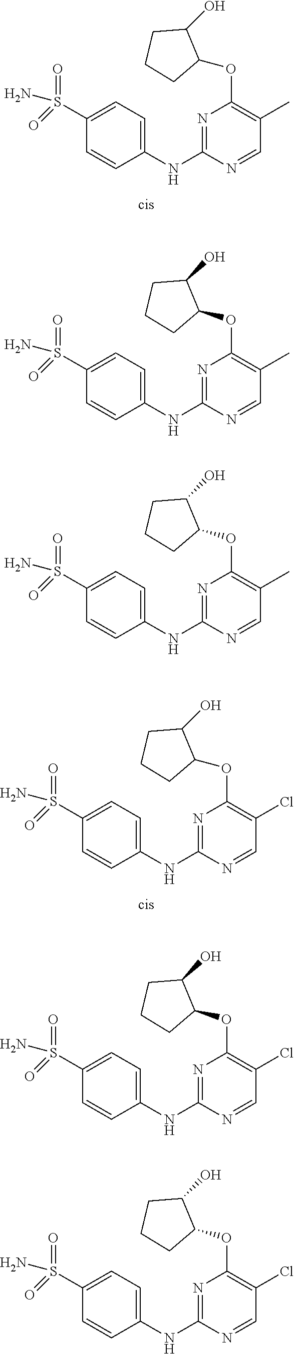 [SMILES: Cc1cnc(Nc2ccc(S(N)(=O)=O)cc2)nc1OC1CCCC1O ; Cc1cnc(Nc2ccc(S(N)(=O)=O)cc2)nc1O[C@H]1CCC[C@H]1O ; Cc1cnc(Nc2ccc(S(N)(=O)=O)cc2)nc1O[C@@H]1CCC[C@@H]1O ; NS(=O)(=O)c1ccc(Nc2ncc(Cl)c(OC3CCCC3O)n2)cc1 ; NS(=O)(=O)c1ccc(Nc2ncc(Cl)c(O[C@H]3CCC[C@H]3O)n2)cc1 ; NS(=O)(=O)c1ccc(Nc2ncc(Cl)c(O[C@@H]3CCC[C@@H]3O)n2)cc1] 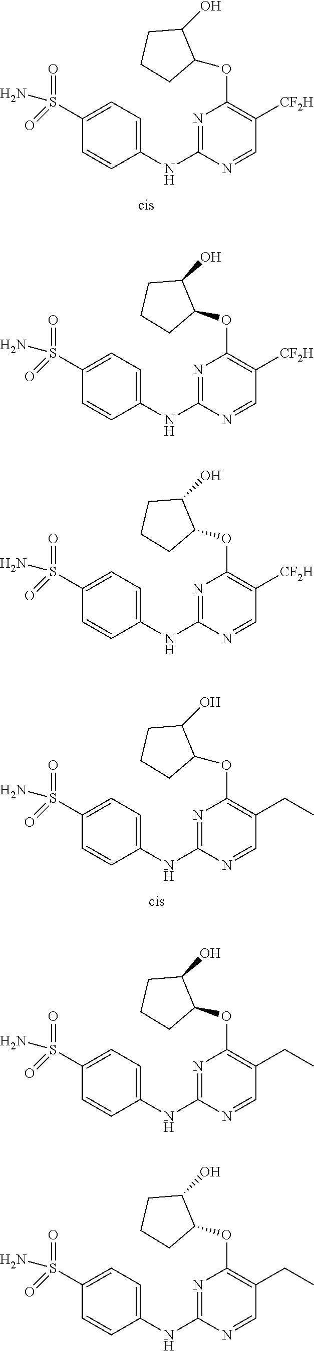 [SMILES: NS(=O)(=O)c1ccc(Nc2ncc(C(F)F)c(OC3CCCC3O)n2)cc1 ; NS(=O)(=O)c1ccc(Nc2ncc(C(F)F)c(O[C@H]3CCC[C@H]3O)n2)cc1 ; NS(=O)(=O)c1ccc(Nc2ncc(C(F)F)c(O[C@@H]3CCC[C@@H]3O)n2)cc1 ; CCc1cnc(Nc2ccc(S(N)(=O)=O)cc2)nc1OC1CCCC1O ; CCc1cnc(Nc2ccc(S(N)(=O)=O)cc2)nc1O[C@H]1CCC[C@H]1O ; CCc1cnc(Nc2ccc(S(N)(=O)=O)cc2)nc1O[C@@H]1CCC[C@@H]1O] 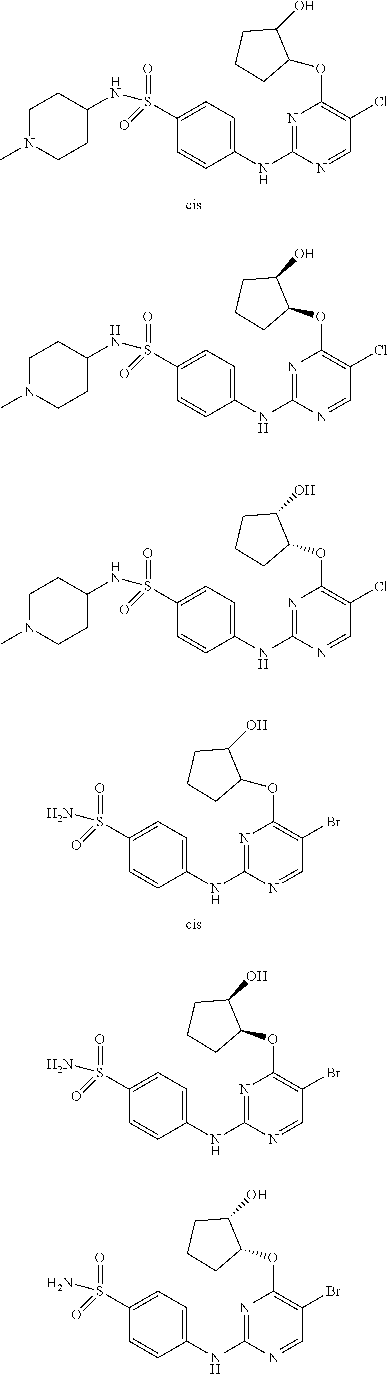 [SMILES: CN1CCC(NS(=O)(=O)c2ccc(Nc3ncc(Cl)c(OC4CCCC4O)n3)cc2)CC1 ; CN1CCC(NS(=O)(=O)c2ccc(Nc3ncc(Cl)c(O[C@H]4CCC[C@H]4O)n3)cc2)CC1 ; CN1CCC(NS(=O)(=O)c2ccc(Nc3ncc(Cl)c(O[C@@H]4CCC[C@@H]4O)n3)cc2)CC1 ; NS(=O)(=O)c1ccc(Nc2ncc(Br)c(OC3CCCC3O)n2)cc1 ; NS(=O)(=O)c1ccc(Nc2ncc(Br)c(O[C@H]3CCC[C@H]3O)n2)cc1 ; NS(=O)(=O)c1ccc(Nc2ncc(Br)c(O[C@@H]3CCC[C@@H]3O)n2)cc1] 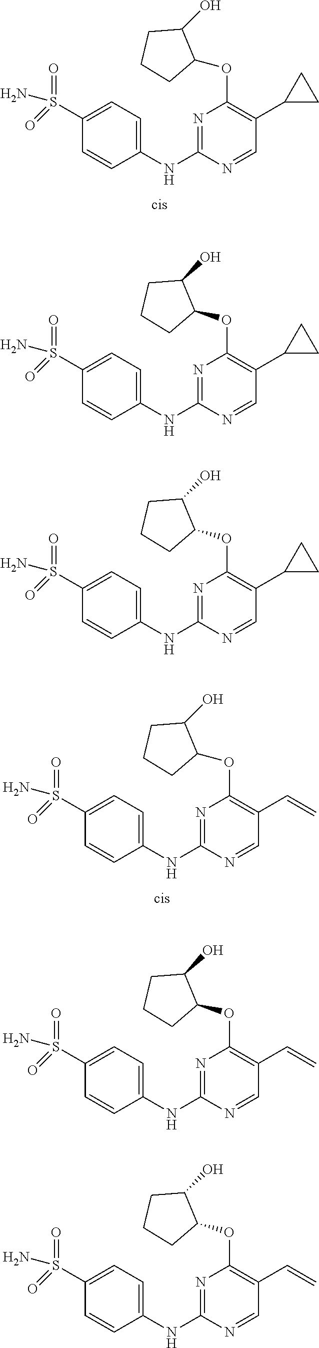 [SMILES: NS(=O)(=O)c1ccc(Nc2ncc(C3CC3)c(OC3CCCC3O)n2)cc1 ; NS(=O)(=O)c1ccc(Nc2ncc(C3CC3)c(O[C@H]3CCC[C@H]3O)n2)cc1 ; NS(=O)(=O)c1ccc(Nc2ncc(C3CC3)c(O[C@@H]3CCC[C@@H]3O)n2)cc1 ; C=Cc1cnc(Nc2ccc(S(N)(=O)=O)cc2)nc1OC1CCCC1O ; C=Cc1cnc(Nc2ccc(S(N)(=O)=O)cc2)nc1O[C@H]1CCC[C@H]1O ; C=Cc1cnc(Nc2ccc(S(N)(=O)=O)cc2)nc1O[C@@H]1CCC[C@@H]1O] 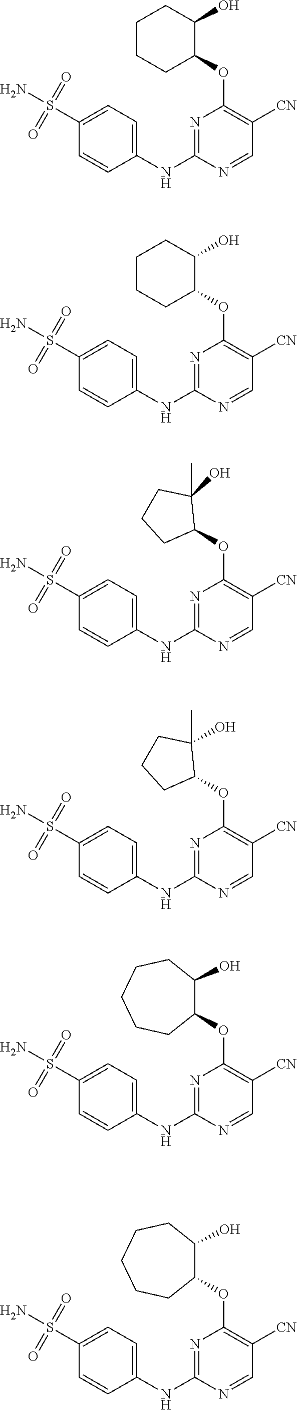 [SMILES: N#Cc1cnc(Nc2ccc(S(N)(=O)=O)cc2)nc1O[C@H]1CCCC[C@H]1O ; N#Cc1cnc(Nc2ccc(S(N)(=O)=O)cc2)nc1O[C@@H]1CCCC[C@@H]1O ; C[C@@]1(O)CCC[C@@H]1Oc1nc(Nc2ccc(S(N)(=O)=O)cc2)ncc1C#N ; C[C@]1(O)CCC[C@H]1Oc1nc(Nc2ccc(S(N)(=O)=O)cc2)ncc1C#N ; N#Cc1cnc(Nc2ccc(S(N)(=O)=O)cc2)nc1O[C@H]1CCCCC[C@H]1O ; N#Cc1cnc(Nc2ccc(S(N)(=O)=O)cc2)nc1O[C@@H]1CCCCC[C@@H]1O] 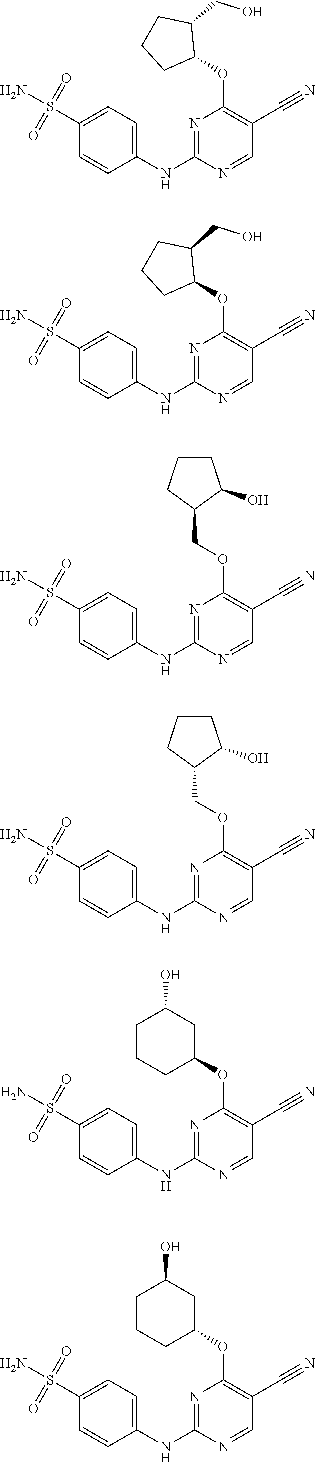 [SMILES: N#Cc1cnc(Nc2ccc(S(N)(=O)=O)cc2)nc1O[C@@H]1CCC[C@@H]1CO ; N#Cc1cnc(Nc2ccc(S(N)(=O)=O)cc2)nc1O[C@H]1CCC[C@H]1CO ; N#Cc1cnc(Nc2ccc(S(N)(=O)=O)cc2)nc1OC[C@H]1CCC[C@H]1O ; N#Cc1cnc(Nc2ccc(S(N)(=O)=O)cc2)nc1OC[C@@H]1CCC[C@@H]1O ; N#Cc1cnc(Nc2ccc(S(N)(=O)=O)cc2)nc1O[C@H]1CCC[C@H](O)C1 ; N#Cc1cnc(Nc2ccc(S(N)(=O)=O)cc2)nc1O[C@@H]1CCC[C@@H](O)C1] 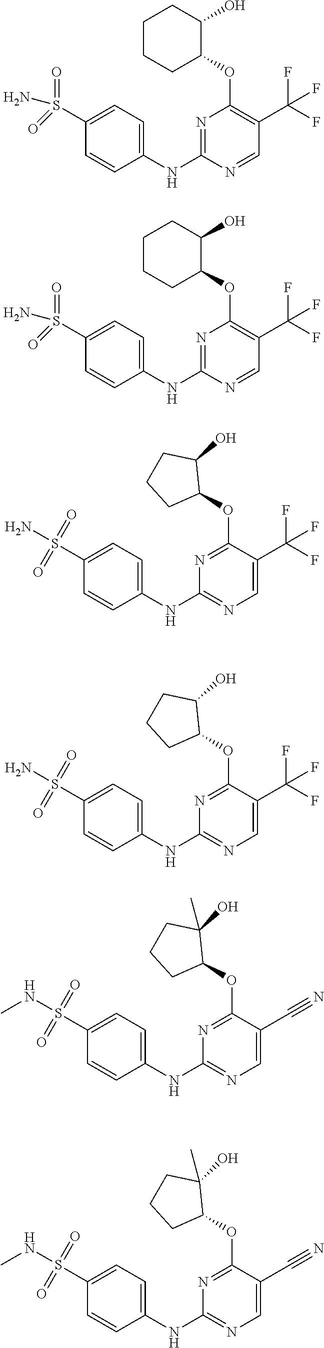 [SMILES: NS(=O)(=O)c1ccc(Nc2ncc(C(F)(F)F)c(O[C@@H]3CCCC[C@@H]3O)n2)cc1 ; NS(=O)(=O)c1ccc(Nc2ncc(C(F)(F)F)c(O[C@H]3CCCC[C@H]3O)n2)cc1 ; NS(=O)(=O)c1ccc(Nc2ncc(C(F)(F)F)c(O[C@H]3CCC[C@H]3O)n2)cc1 ; NS(=O)(=O)c1ccc(Nc2ncc(C(F)(F)F)c(O[C@@H]3CCC[C@@H]3O)n2)cc1 ; CNS(=O)(=O)c1ccc(Nc2ncc(C#N)c(O[C@H]3CCC[C@@]3(C)O)n2)cc1 ; CNS(=O)(=O)c1ccc(Nc2ncc(C#N)c(O[C@@H]3CCC[C@]3(C)O)n2)cc1] 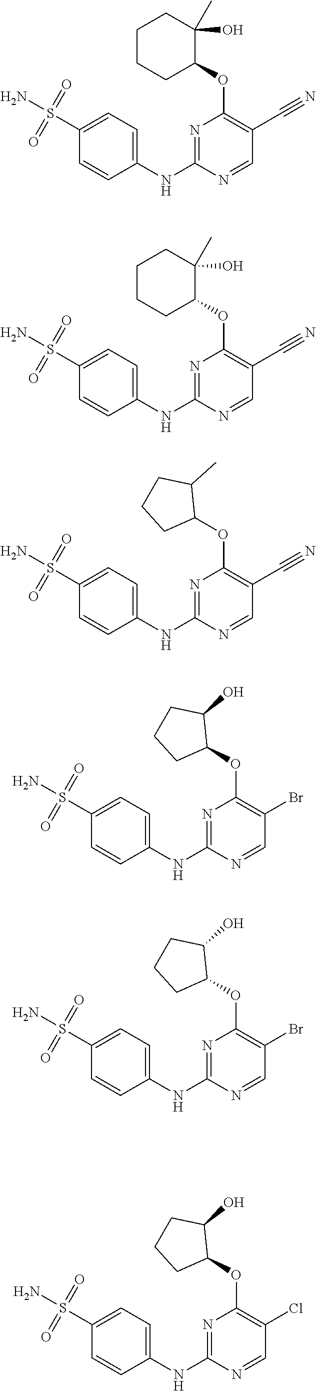 [SMILES: C[C@@]1(O)CCCC[C@@H]1Oc1nc(Nc2ccc(S(N)(=O)=O)cc2)ncc1C#N ; C[C@]1(O)CCCC[C@H]1Oc1nc(Nc2ccc(S(N)(=O)=O)cc2)ncc1C#N ; CC1CCCC1Oc1nc(Nc2ccc(S(N)(=O)=O)cc2)ncc1C#N ; NS(=O)(=O)c1ccc(Nc2ncc(Br)c(O[C@H]3CCC[C@H]3O)n2)cc1 ; NS(=O)(=O)c1ccc(Nc2ncc(Br)c(O[C@@H]3CCC[C@@H]3O)n2)cc1 ; NS(=O)(=O)c1ccc(Nc2ncc(Cl)c(O[C@H]3CCC[C@H]3O)n2)cc1] 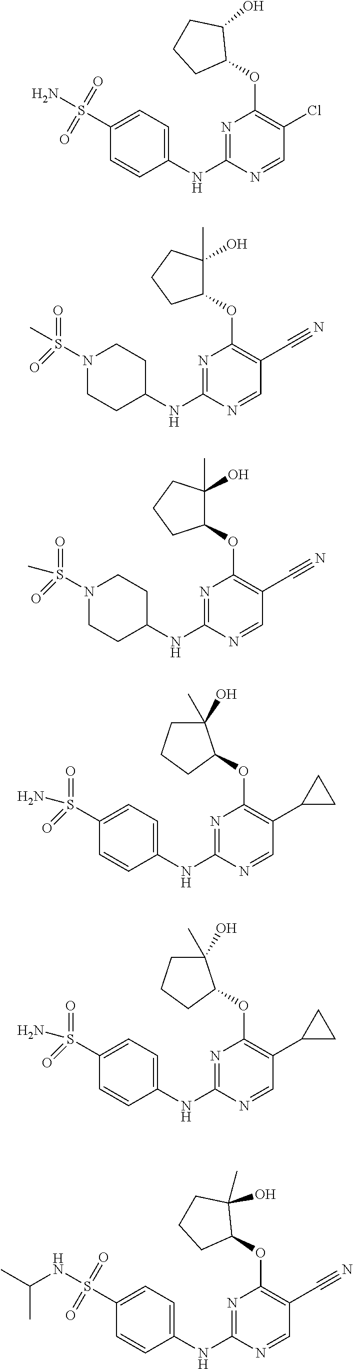 [SMILES: NS(=O)(=O)c1ccc(Nc2ncc(Cl)c(O[C@@H]3CCC[C@@H]3O)n2)cc1 ; C[C@]1(O)CCC[C@H]1Oc1nc(NC2CCN(S(C)(=O)=O)CC2)ncc1C#N ; C[C@@]1(O)CCC[C@@H]1Oc1nc(NC2CCN(S(C)(=O)=O)CC2)ncc1C#N ; C[C@@]1(O)CCC[C@@H]1Oc1nc(Nc2ccc(S(N)(=O)=O)cc2)ncc1C1CC1 ; C[C@]1(O)CCC[C@H]1Oc1nc(Nc2ccc(S(N)(=O)=O)cc2)ncc1C1CC1 ; CC(C)NS(=O)(=O)c1ccc(Nc2ncc(C#N)c(O[C@H]3CCC[C@@]3(C)O)n2)cc1] 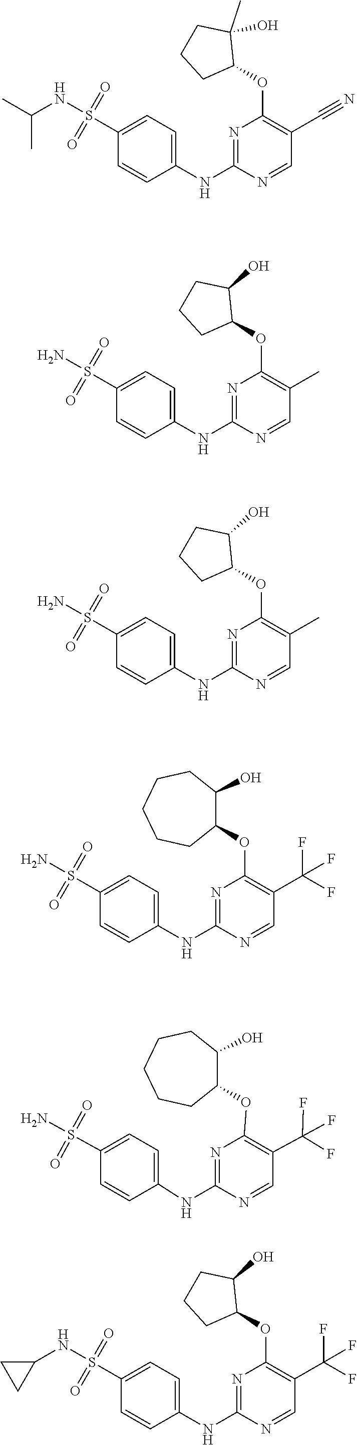 [SMILES: CC(C)NS(=O)(=O)c1ccc(Nc2ncc(C#N)c(O[C@@H]3CCC[C@]3(C)O)n2)cc1 ; Cc1cnc(Nc2ccc(S(N)(=O)=O)cc2)nc1O[C@H]1CCC[C@H]1O ; Cc1cnc(Nc2ccc(S(N)(=O)=O)cc2)nc1O[C@@H]1CCC[C@@H]1O ; NS(=O)(=O)c1ccc(Nc2ncc(C(F)(F)F)c(O[C@H]3CCCCC[C@H]3O)n2)cc1 ; NS(=O)(=O)c1ccc(Nc2ncc(C(F)(F)F)c(O[C@@H]3CCCCC[C@@H]3O)n2)cc1 ; O=S(=O)(NC1CC1)c1ccc(Nc2ncc(C(F)(F)F)c(O[C@H]3CCC[C@H]3O)n2)cc1] 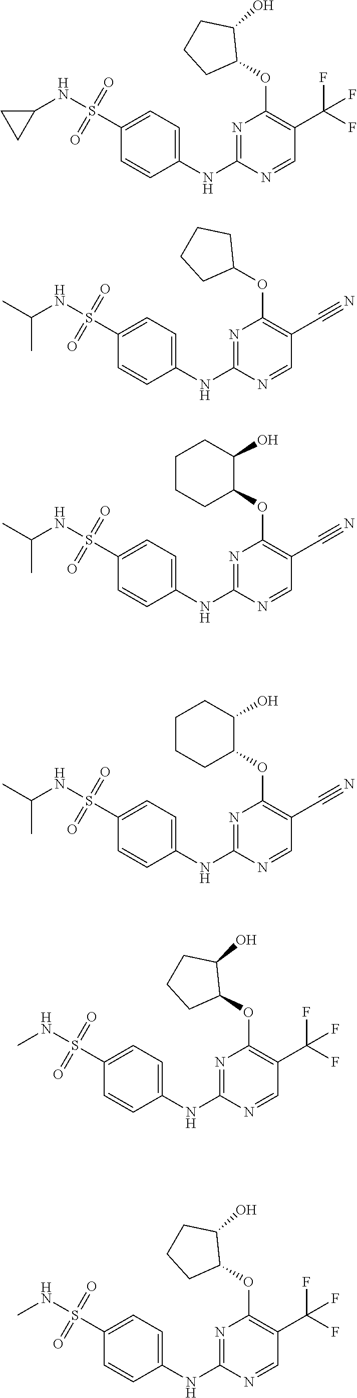 [SMILES: O=S(=O)(NC1CC1)c1ccc(Nc2ncc(C(F)(F)F)c(O[C@@H]3CCC[C@@H]3O)n2)cc1 ; CC(C)NS(=O)(=O)c1ccc(Nc2ncc(C#N)c(OC3CCCC3)n2)cc1 ; CC(C)NS(=O)(=O)c1ccc(Nc2ncc(C#N)c(O[C@H]3CCCC[C@H]3O)n2)cc1 ; CC(C)NS(=O)(=O)c1ccc(Nc2ncc(C#N)c(O[C@@H]3CCCC[C@@H]3O)n2)cc1 ; CNS(=O)(=O)c1ccc(Nc2ncc(C(F)(F)F)c(O[C@H]3CCC[C@H]3O)n2)cc1 ; CNS(=O)(=O)c1ccc(Nc2ncc(C(F)(F)F)c(O[C@@H]3CCC[C@@H]3O)n2)cc1] 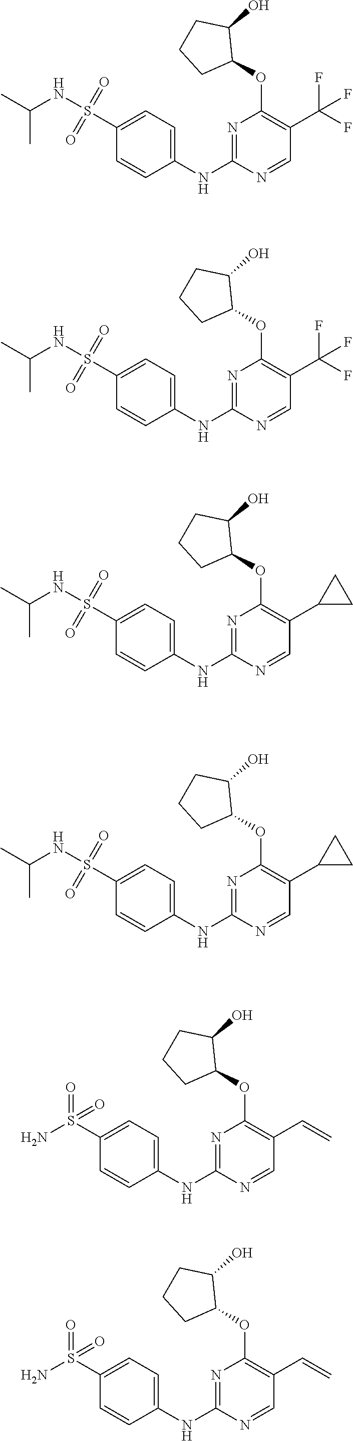 [SMILES: CC(C)NS(=O)(=O)c1ccc(Nc2ncc(C(F)(F)F)c(O[C@H]3CCC[C@H]3O)n2)cc1 ; CC(C)NS(=O)(=O)c1ccc(Nc2ncc(C(F)(F)F)c(O[C@@H]3CCC[C@@H]3O)n2)cc1 ; CC(C)NS(=O)(=O)c1ccc(Nc2ncc(C3CC3)c(O[C@H]3CCC[C@H]3O)n2)cc1 ; CC(C)NS(=O)(=O)c1ccc(Nc2ncc(C3CC3)c(O[C@@H]3CCC[C@@H]3O)n2)cc1 ; C=Cc1cnc(Nc2ccc(S(N)(=O)=O)cc2)nc1O[C@H]1CCC[C@H]1O ; C=Cc1cnc(Nc2ccc(S(N)(=O)=O)cc2)nc1O[C@@H]1CCC[C@@H]1O] 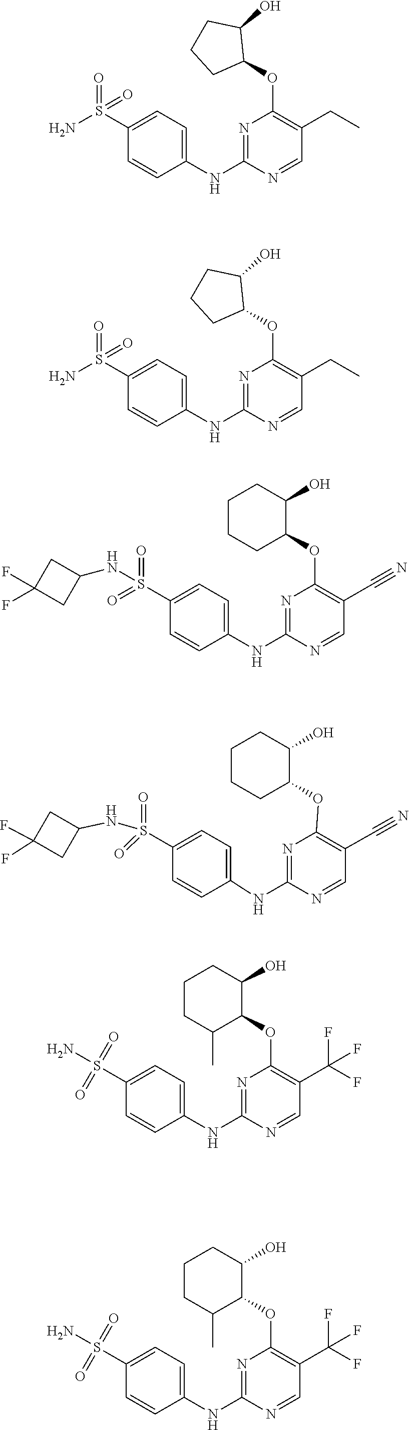 [SMILES: CCc1cnc(Nc2ccc(S(N)(=O)=O)cc2)nc1O[C@H]1CCC[C@H]1O ; CCc1cnc(Nc2ccc(S(N)(=O)=O)cc2)nc1O[C@@H]1CCC[C@@H]1O ; N#Cc1cnc(Nc2ccc(S(=O)(=O)NC3CC(F)(F)C3)cc2)nc1O[C@H]1CCCC[C@H]1O ; N#Cc1cnc(Nc2ccc(S(=O)(=O)NC3CC(F)(F)C3)cc2)nc1O[C@@H]1CCCC[C@@H]1O ; CC1CCC[C@@H](O)[C@H]1Oc1nc(Nc2ccc(S(N)(=O)=O)cc2)ncc1C(F)(F)F ; CC1CCC[C@H](O)[C@@H]1Oc1nc(Nc2ccc(S(N)(=O)=O)cc2)ncc1C(F)(F)F] 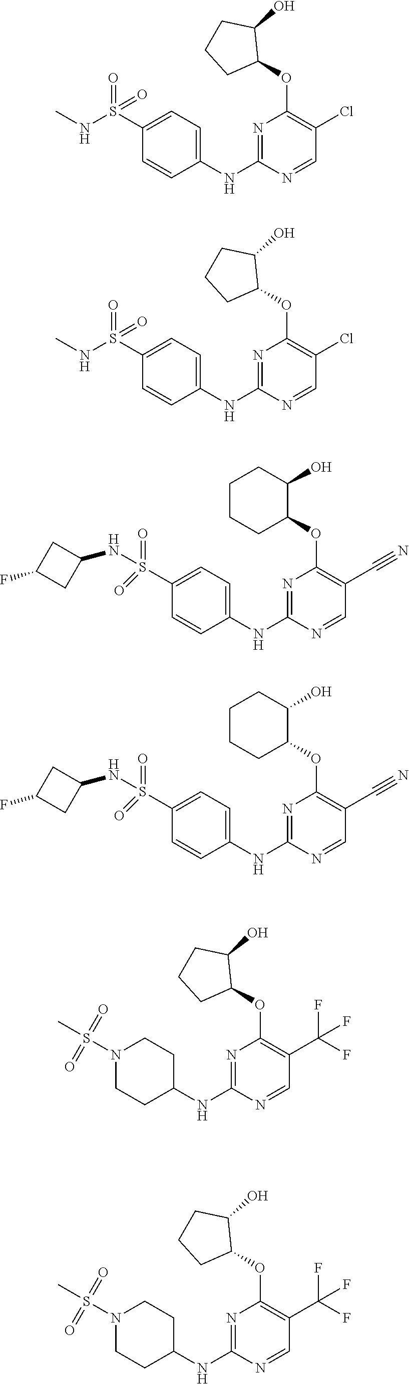 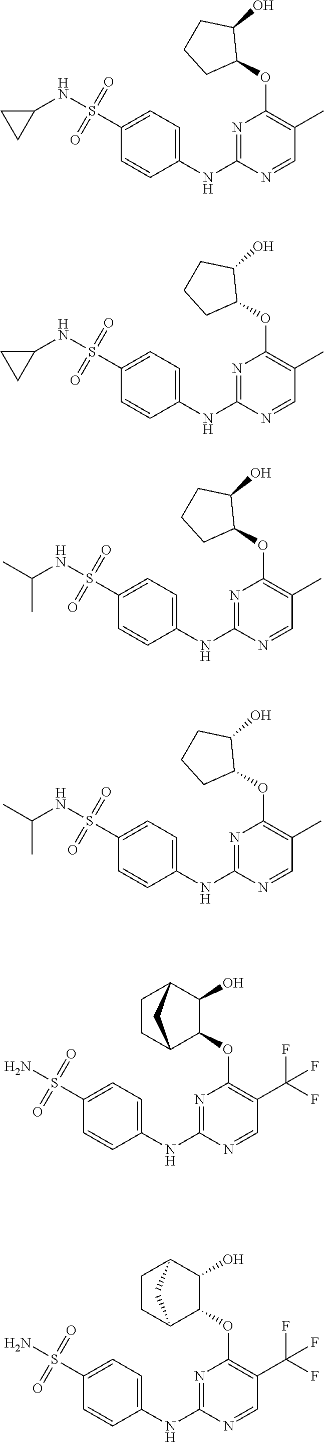 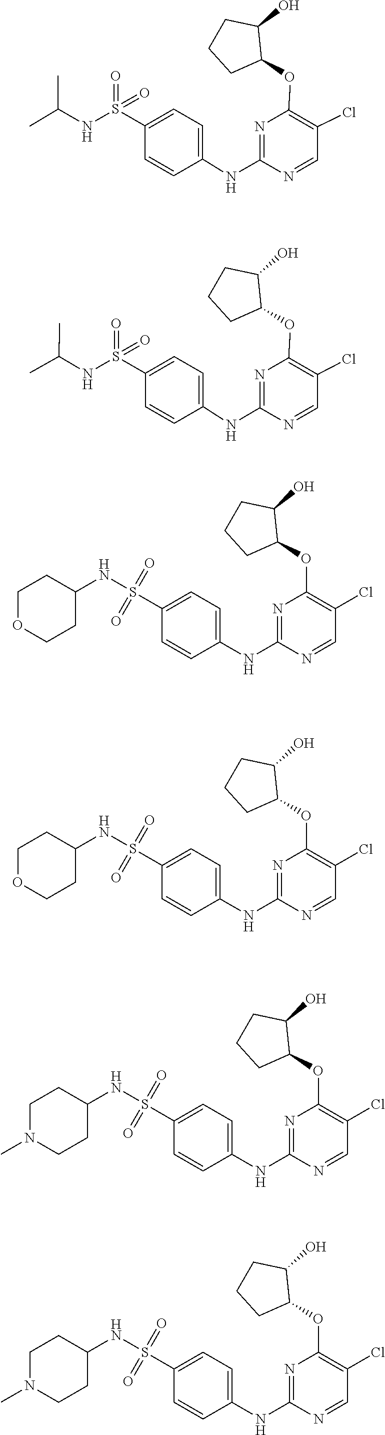 [SMILES: CC(C)NS(=O)(=O)c1ccc(Nc2ncc(Cl)c(O[C@H]3CCC[C@H]3O)n2)cc1 ; CC(C)NS(=O)(=O)c1ccc(Nc2ncc(Cl)c(O[C@@H]3CCC[C@@H]3O)n2)cc1 ; O=S(=O)(NC1CCOCC1)c1ccc(Nc2ncc(Cl)c(O[C@H]3CCC[C@H]3O)n2)cc1 ; O=S(=O)(NC1CCOCC1)c1ccc(Nc2ncc(Cl)c(O[C@@H]3CCC[C@@H]3O)n2)cc1 ; CN1CCC(NS(=O)(=O)c2ccc(Nc3ncc(Cl)c(O[C@H]4CCC[C@H]4O)n3)cc2)CC1 ; CN1CCC(NS(=O)(=O)c2ccc(Nc3ncc(Cl)c(O[C@@H]4CCC[C@@H]4O)n3)cc2)CC1] 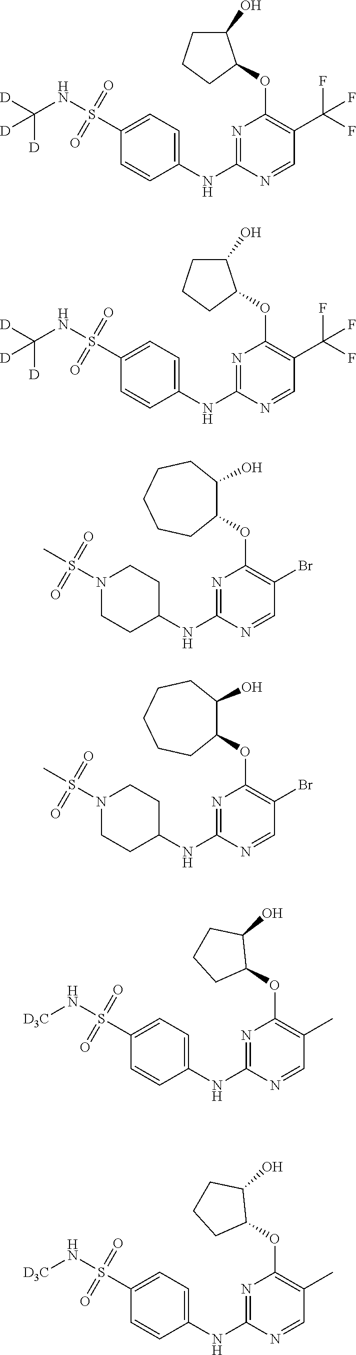 [SMILES: [2H]C([2H])([2H])NS(=O)(=O)c1ccc(Nc2ncc(C(F)(F)F)c(O[C@H]3CCC[C@H]3O)n2)cc1 ; [2H]C([2H])([2H])NS(=O)(=O)c1ccc(Nc2ncc(C(F)(F)F)c(O[C@@H]3CCC[C@@H]3O)n2)cc1 ; CS(=O)(=O)N1CCC(Nc2ncc(Br)c(O[C@@H]3CCCCC[C@@H]3O)n2)CC1 ; CS(=O)(=O)N1CCC(Nc2ncc(Br)c(O[C@H]3CCCCC[C@H]3O)n2)CC1 ; [2H]C([2H])([2H])NS(=O)(=O)c1ccc(Nc2ncc(C)c(O[C@H]3CCC[C@H]3O)n2)cc1 ; [2H]C([2H])([2H])NS(=O)(=O)c1ccc(Nc2ncc(C)c(O[C@@H]3CCC[C@@H]3O)n2)cc1] 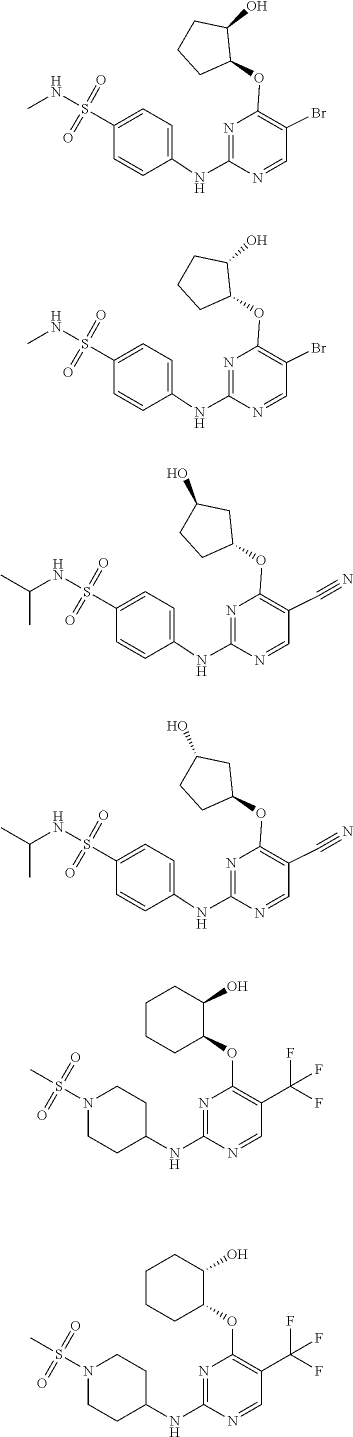 [SMILES: CNS(=O)(=O)c1ccc(Nc2ncc(Br)c(O[C@H]3CCC[C@H]3O)n2)cc1 ; CNS(=O)(=O)c1ccc(Nc2ncc(Br)c(O[C@@H]3CCC[C@@H]3O)n2)cc1 ; CC(C)NS(=O)(=O)c1ccc(Nc2ncc(C#N)c(O[C@@H]3CC[C@@H](O)C3)n2)cc1 ; CC(C)NS(=O)(=O)c1ccc(Nc2ncc(C#N)c(O[C@H]3CC[C@H](O)C3)n2)cc1 ; CS(=O)(=O)N1CCC(Nc2ncc(C(F)(F)F)c(O[C@H]3CCCC[C@H]3O)n2)CC1 ; CS(=O)(=O)N1CCC(Nc2ncc(C(F)(F)F)c(O[C@@H]3CCCC[C@@H]3O)n2)CC1] 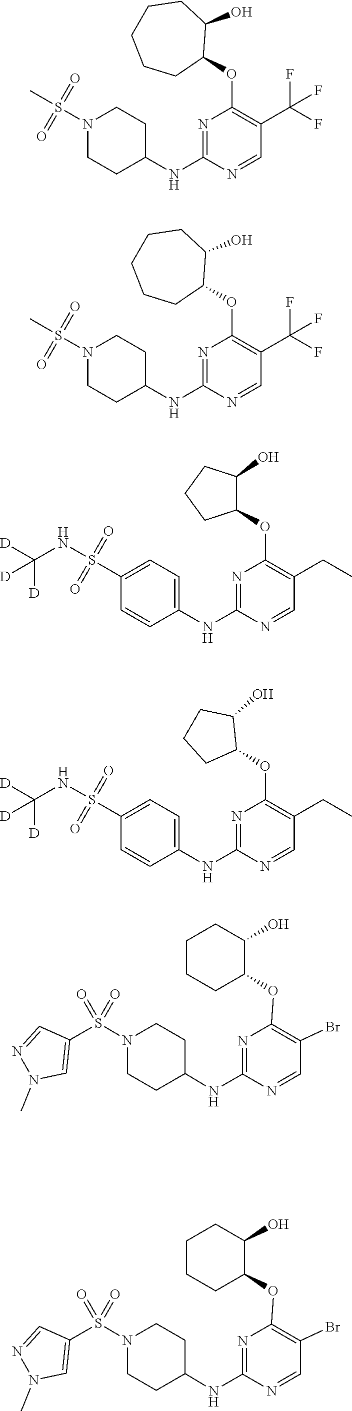 [SMILES: CS(=O)(=O)N1CCC(Nc2ncc(C(F)(F)F)c(O[C@H]3CCCCC[C@H]3O)n2)CC1 ; CS(=O)(=O)N1CCC(Nc2ncc(C(F)(F)F)c(O[C@@H]3CCCCC[C@@H]3O)n2)CC1 ; [2H]C([2H])([2H])NS(=O)(=O)c1ccc(Nc2ncc(CC)c(O[C@H]3CCC[C@H]3O)n2)cc1 ; [2H]C([2H])([2H])NS(=O)(=O)c1ccc(Nc2ncc(CC)c(O[C@@H]3CCC[C@@H]3O)n2)cc1 ; Cn1cc(S(=O)(=O)N2CCC(Nc3ncc(Br)c(O[C@@H]4CCCC[C@@H]4O)n3)CC2)cn1 ; Cn1cc(S(=O)(=O)N2CCC(Nc3ncc(Br)c(O[C@H]4CCCC[C@H]4O)n3)CC2)cn1] 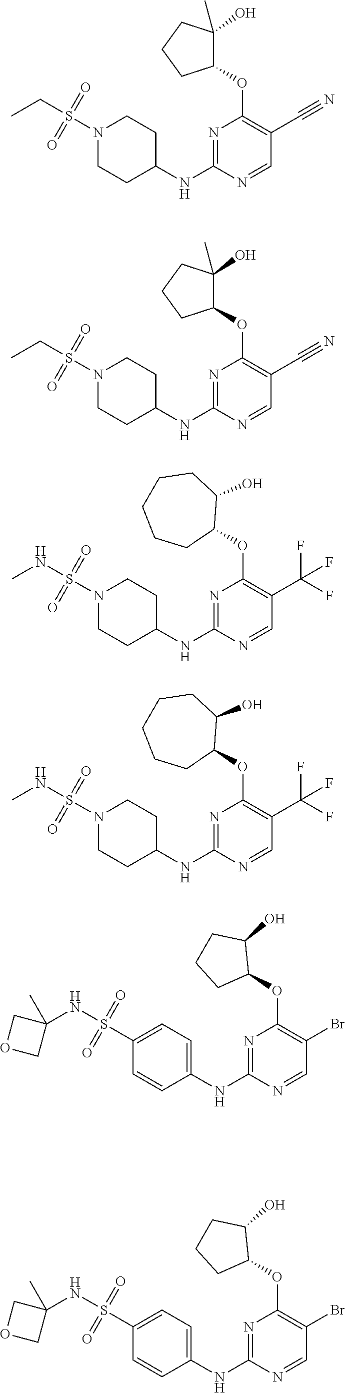 [SMILES: CCS(=O)(=O)N1CCC(Nc2ncc(C#N)c(O[C@@H]3CCC[C@]3(C)O)n2)CC1 ; CCS(=O)(=O)N1CCC(Nc2ncc(C#N)c(O[C@H]3CCC[C@@]3(C)O)n2)CC1 ; CNS(=O)(=O)N1CCC(Nc2ncc(C(F)(F)F)c(O[C@@H]3CCCCC[C@@H]3O)n2)CC1 ; CNS(=O)(=O)N1CCC(Nc2ncc(C(F)(F)F)c(O[C@H]3CCCCC[C@H]3O)n2)CC1 ; CC1(NS(=O)(=O)c2ccc(Nc3ncc(Br)c(O[C@H]4CCC[C@H]4O)n3)cc2)COC1 ; CC1(NS(=O)(=O)c2ccc(Nc3ncc(Br)c(O[C@@H]4CCC[C@@H]4O)n3)cc2)COC1] 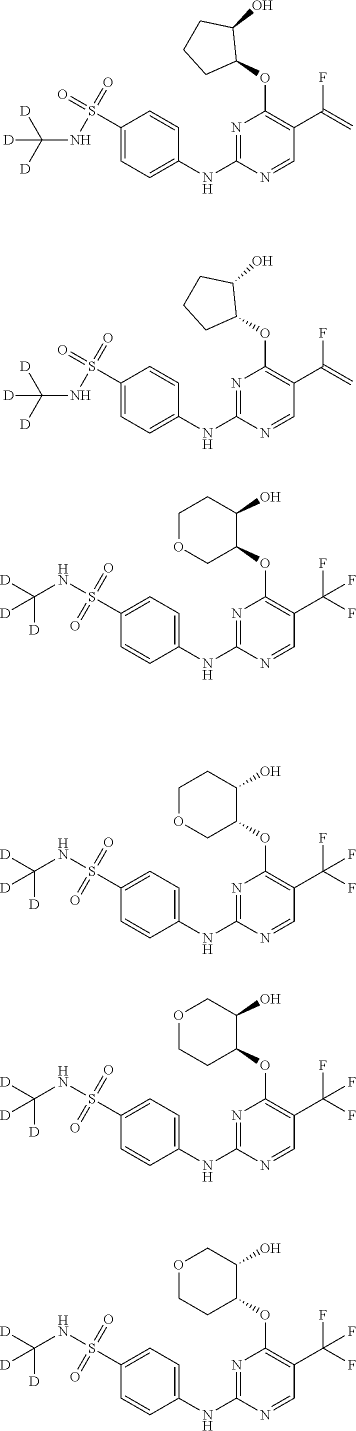 [SMILES: [2H]C([2H])([2H])NS(=O)(=O)c1ccc(Nc2ncc(C(=C)F)c(O[C@H]3CCC[C@H]3O)n2)cc1 ; [2H]C([2H])([2H])NS(=O)(=O)c1ccc(Nc2ncc(C(=C)F)c(O[C@@H]3CCC[C@@H]3O)n2)cc1 ; [2H]C([2H])([2H])NS(=O)(=O)c1ccc(Nc2ncc(C(F)(F)F)c(O[C@H]3COCC[C@H]3O)n2)cc1 ; [2H]C([2H])([2H])NS(=O)(=O)c1ccc(Nc2ncc(C(F)(F)F)c(O[C@@H]3COCC[C@@H]3O)n2)cc1 ; [2H]C([2H])([2H])NS(=O)(=O)c1ccc(Nc2ncc(C(F)(F)F)c(O[C@H]3CCOC[C@H]3O)n2)cc1 ; [2H]C([2H])([2H])NS(=O)(=O)c1ccc(Nc2ncc(C(F)(F)F)c(O[C@@H]3CCOC[C@@H]3O)n2)cc1] 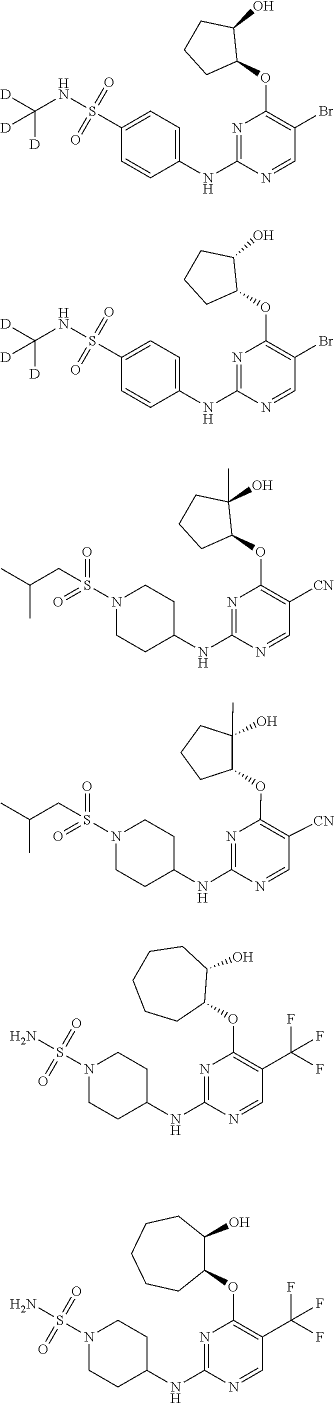 [SMILES: [2H]C([2H])([2H])NS(=O)(=O)c1ccc(Nc2ncc(Br)c(O[C@H]3CCC[C@H]3O)n2)cc1 ; [2H]C([2H])([2H])NS(=O)(=O)c1ccc(Nc2ncc(Br)c(O[C@@H]3CCC[C@@H]3O)n2)cc1 ; CC(C)CS(=O)(=O)N1CCC(Nc2ncc(C#N)c(O[C@H]3CCC[C@@]3(C)O)n2)CC1 ; CC(C)CS(=O)(=O)N1CCC(Nc2ncc(C#N)c(O[C@@H]3CCC[C@]3(C)O)n2)CC1 ; NS(=O)(=O)N1CCC(Nc2ncc(C(F)(F)F)c(O[C@@H]3CCCCC[C@@H]3O)n2)CC1 ; NS(=O)(=O)N1CCC(Nc2ncc(C(F)(F)F)c(O[C@H]3CCCCC[C@H]3O)n2)CC1] 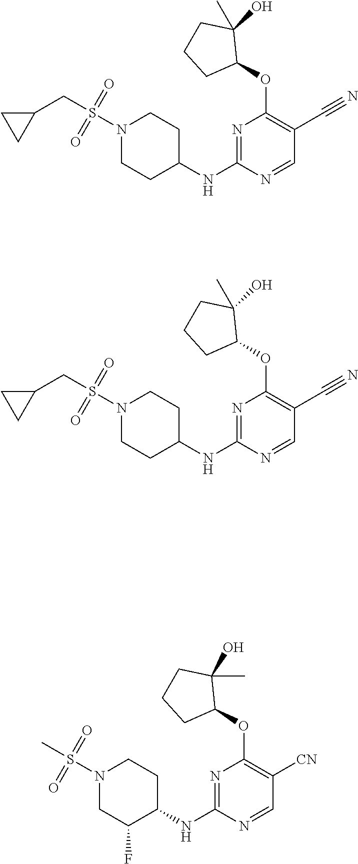 [SMILES: C[C@@]1(O)CCC[C@@H]1Oc1nc(NC2CCN(S(=O)(=O)CC3CC3)CC2)ncc1C#N ; C[C@]1(O)CCC[C@H]1Oc1nc(NC2CCN(S(=O)(=O)CC3CC3)CC2)ncc1C#N ; C[C@@]1(O)CCC[C@@H]1Oc1nc(N[C@H]2CCN(S(C)(=O)=O)C[C@H]2F)ncc1C#N] 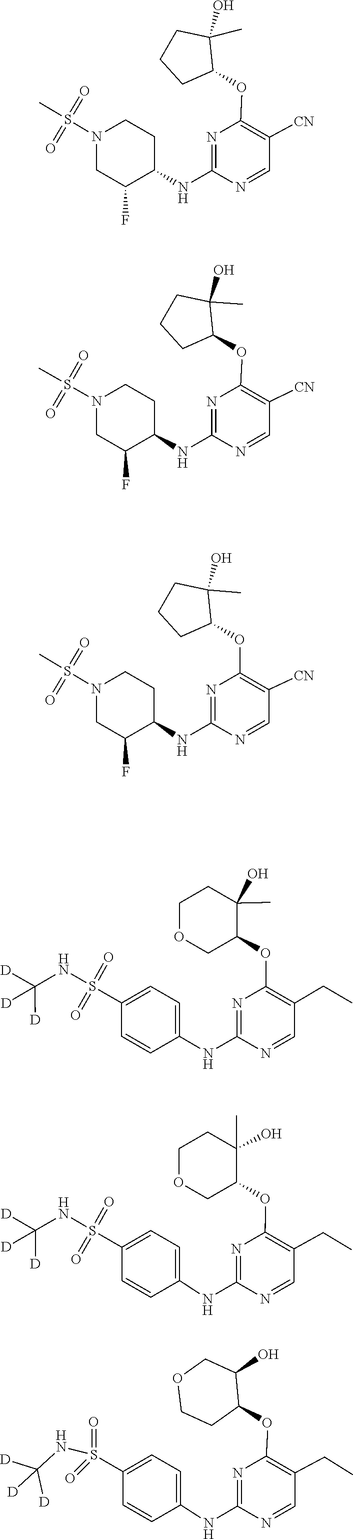 [SMILES: C[C@]1(O)CCC[C@H]1Oc1nc(N[C@H]2CCN(S(C)(=O)=O)C[C@H]2F)ncc1C#N ; C[C@@]1(O)CCC[C@@H]1Oc1nc(N[C@@H]2CCN(S(C)(=O)=O)C[C@@H]2F)ncc1C#N ; C[C@]1(O)CCC[C@H]1Oc1nc(N[C@@H]2CCN(S(C)(=O)=O)C[C@@H]2F)ncc1C#N ; [2H]C([2H])([2H])NS(=O)(=O)c1ccc(Nc2ncc(CC)c(O[C@H]3COCC[C@@]3(C)O)n2)cc1 ; [2H]C([2H])([2H])NS(=O)(=O)c1ccc(Nc2ncc(CC)c(O[C@@H]3COCC[C@]3(C)O)n2)cc1 ; [2H]C([2H])([2H])NS(=O)(=O)c1ccc(Nc2ncc(CC)c(O[C@H]3CCOC[C@H]3O)n2)cc1] 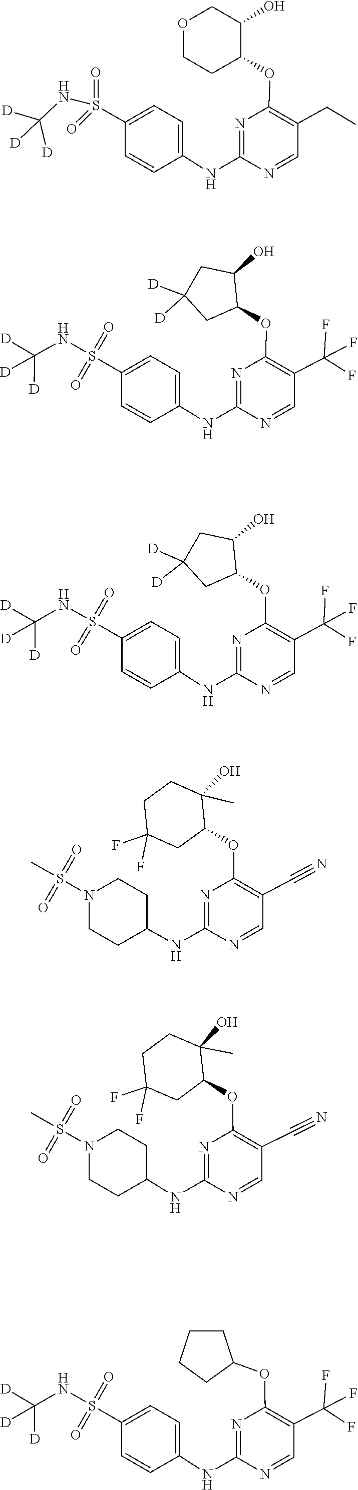 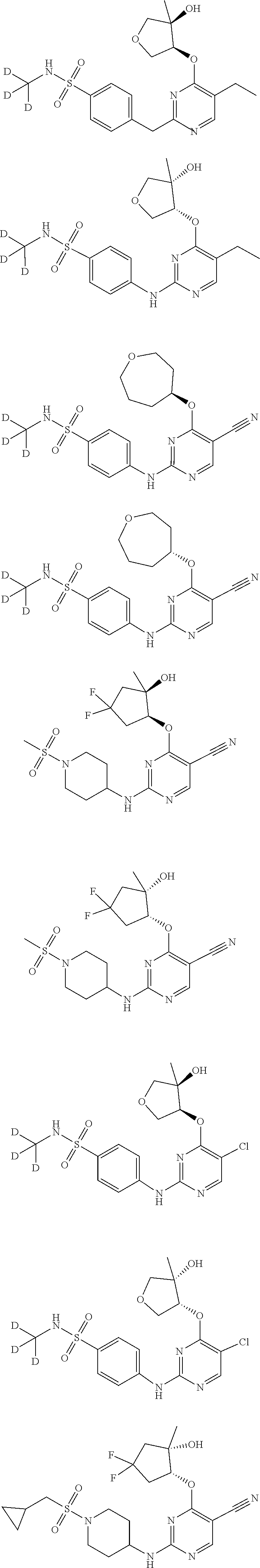 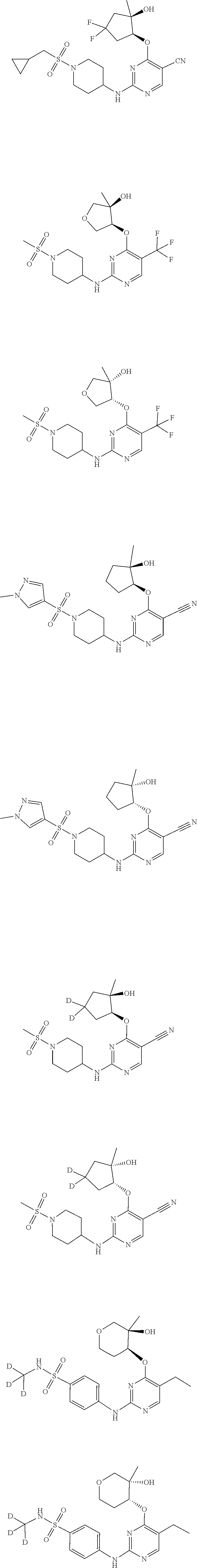 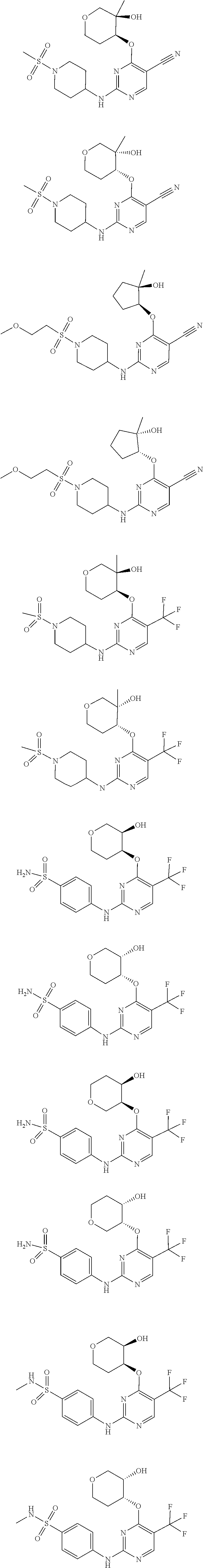 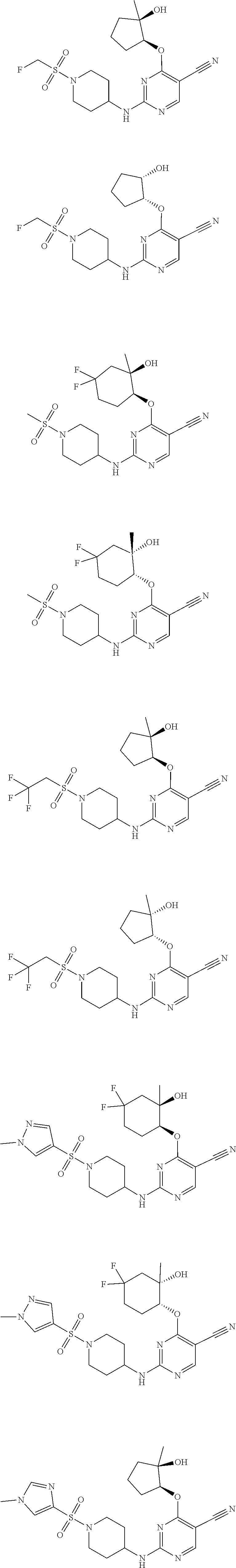 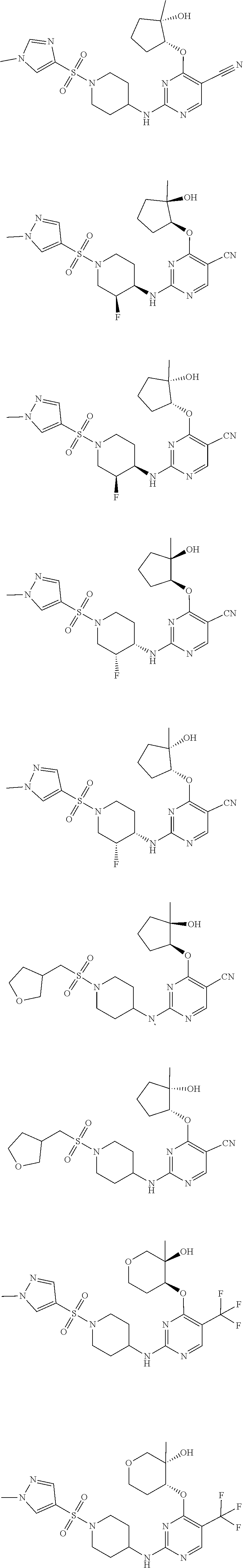 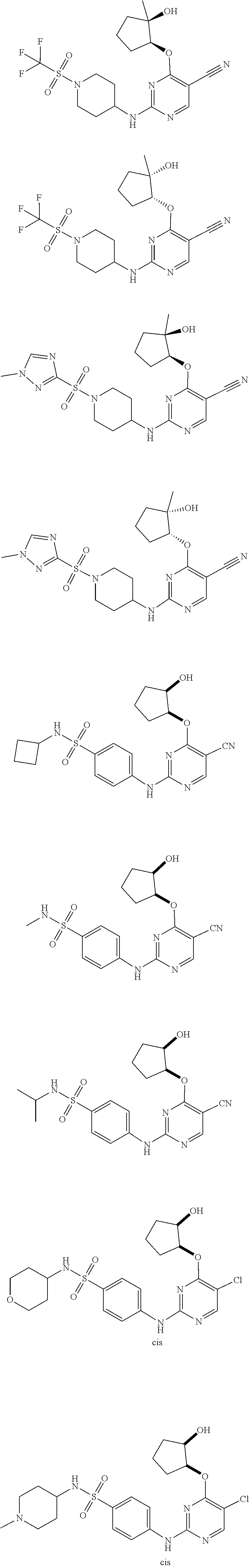 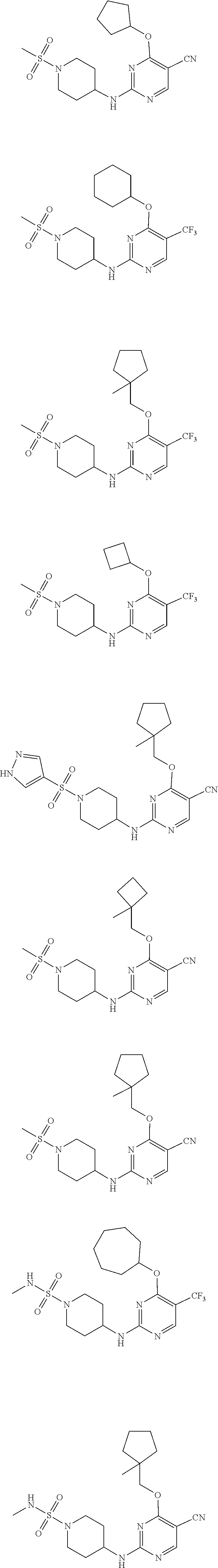 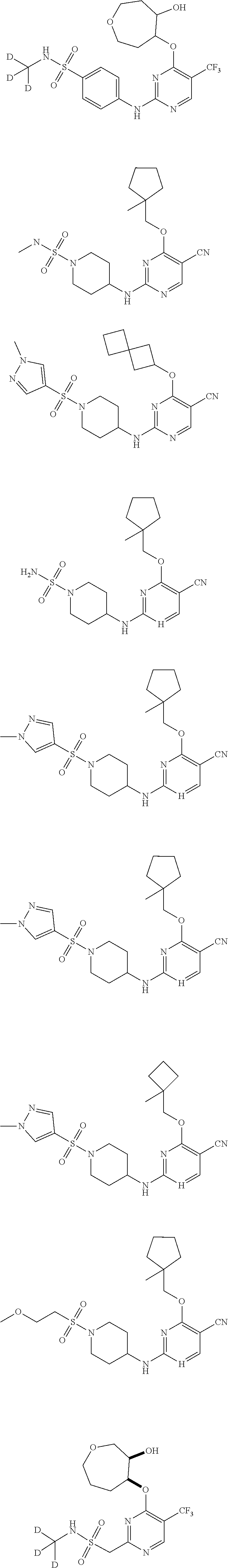 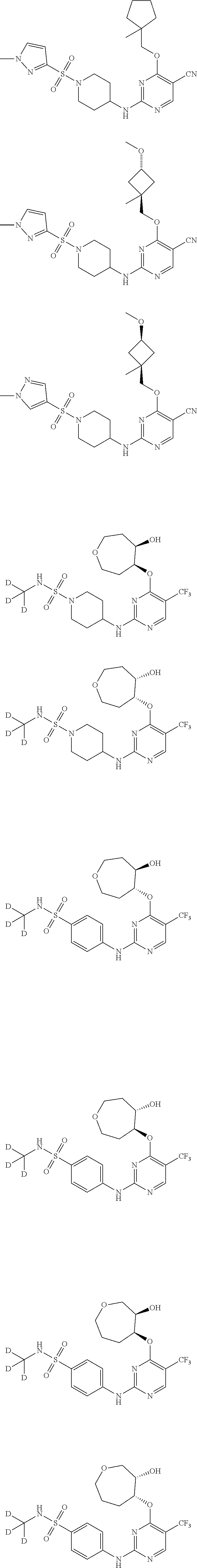 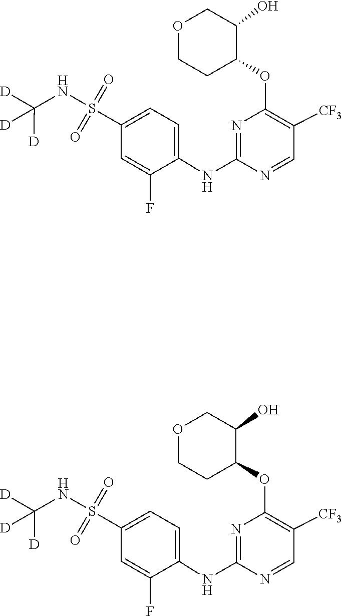 [SMILES: [2H]C([2H])([2H])NS(=O)(=O)c1ccc(Nc2ncc(C(F)(F)F)c(O[C@@H]3CCOC[C@@H]3O)n2)c(F)c1 ; [2H]C([2H])([2H])NS(=O)(=O)c1ccc(Nc2ncc(C(F)(F)F)c(O[C@H]3CCOC[C@H]3O)n2)c(F)c1]
or a stereoisomer thereof, a deuterated analog thereof, or a pharmaceutically acceptable salt thereof.

98. A pharmaceutical composition comprising the compound of claim 1, or a pharmaceutically acceptable salt thereof, and a pharmaceutically acceptable excipient.

99. A method of treating cancer in a subject in need thereof, the method comprising administering to the subject a therapeutically effective amount of the compound of claim 1.

100. The method of claim 99, wherein the cancer is breast cancer, ovarian cancer, bladder cancer, uterine cancer, prostate cancer, lung cancer, esophageal cancer, head and neck cancer, colorectal cancer, kidney cancer, liver cancer, pancreatic cancer, stomach cancer, thyroid cancer, or a combination thereof.

101. The method of claim 100, wherein the breast cancer is is selected from advanced breast cancer, metastatic breast cancer; ER-positive/HR-positive breast cancer; HER2-negative breast cancer; ER-positive/HR-positive, HER2-positive breast cancer; triple negative breast cancer (TNBC); inflammatory breast cancer, endocrine resistant breast cancer, trastuzumab resistant breast cancer; or breast cancer demonstrating primary or acquired resistance to CDK4/CDK6 inhibition.

102-104. (canceled)

105. The method of claim 99, wherein the cancer is characterized by an amplification or overexpression of cyclin E1 and/or cyclin E2.
